(19) United States
(12) Patent Application Publication
(10) Pub. No.: US 2024/0376128 A1
(43) Pub. Date: Nov. 14, 2024

(54) N-CYCLOPROPYLPYRIDO [4, 3-D] PYRIMIDIN-4-AMINE DERIVATIVES AND USES THEREOF

(71) Applicant: Jacobio Pharmaceuticals Co., Ltd., Beijing (CN)

(72) Inventors: Hongwei YANG, Beijing (CN); Cunbo MA, Beijing (CN); Panliang GAO, Beijing (CN); Huifeng HAN, Beijing (CN); Peng WANG, Beijing (CN); Runze LI, Beijing (CN); Xiaoyu LIU, Beijing (CN); Yanping WANG, Beijing (CN); Wei LONG, Beijing (CN); Wei ZHANG, Beijing (CN)

(21) Appl. No.: 18/684,203

(22) PCT Filed: Aug. 17, 2022
(86) PCT No.: PCT/CN2022/112918

§ 371 Date: Feb. 16, 2024

(30) Foreign Application Priority Data
Aug. 18, 2021 (WO) PCT/CN2021/113365
Nov. 22, 2021 (WO) PCT/CN2021/132066
Nov. 24, 2021 (WO) PCT/CN2021/132636
Dec. 17, 2021 (WO) PCT/CN2021/139165
Jan. 18, 2022 (WO) PCT/CN2022/072459
Jan. 20, 2022 (WO) PCT/CN2022/072926
Jan. 26, 2022 (WO) PCT/CN2022/074053
Jan. 27, 2022 (WO) PCT/CN2022/074165
Feb. 24, 2022 (WO) PCT/CN2022/077674
Mar. 18, 2022 (WO) PCT/CN2022/081602
Mar. 28, 2022 (WO) PCT/CN2022/083320
Mar. 31, 2022 (WO) PCT/CN2022/084317
Apr. 18, 2022 (WO) PCT/CN2022/087377

(51) Int. Cl. C07D 519/00; A61K 31/519; A61K 31/675; C07F 9/6561

(52) U.S. Cl. CPC C07D 519/00; A61K 31/519; A61K 31/675; C07F 9/6561

(57) ABSTRACT

Provided herein is a compound of Formula (IB), a stereoisomer thereof, a pharmaceutically acceptable salt thereof, a pharmaceutically acceptable salt of the stereoisomer thereof, a prodrug thereof, a deuterated molecule thereof or a conjugated form thereof; composition containing the same and the use thereof.

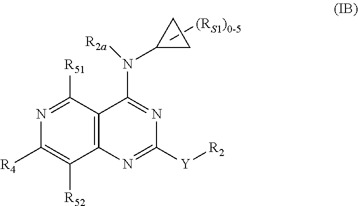

### CROSS-REFERENCES TO RELATED APPLICATIONS

This application claims the benefit of priority to PCT/CN2021/113365, filed on Aug. 18, 2021; PCT/CN2021/132066, filed on Nov. 22, 2021; PCT/CN2021/132636, filed on Nov. 24, 2021; PCT/CN2021/139165, filed on Dec. 17, 2021; PCT/CN2022/072459, filed on Jan. 18, 2022; PCT/CN2022/072926, filed on Jan. 20, 2022; PCT/CN2022/074053, filed on Jan. 26, 2022; PCT/CN2022/074165, filed on Jan. 27, 2022; PCT/CN2022/077674, filed on Feb. 24, 2022; PCT/CN2022/081602, filed on Mar. 18, 2022; PCT/CN2022/083320, filed on Mar. 28, 2022; PCT/CN2022/084317, filed on Mar. 31, 2022 and PCT/CN2022/087377, filed on Apr. 18, 2022, all of which are hereby incorporated herein by reference in their entireties.

### TECHNICAL FIELD

The invention relates to compounds that inhibit the activity of multiple forms of K-Ras protein including K-Ras wild type and K-Ras mutant types, compositions comprising the same, and the methods of using the same.

### BACKGROUND ART

Kirsten Rat Sarcoma 2 Viral Oncogene Homolog (“K-Ras”) is a small GTPase and a member of the RAS family of oncogenes. K-Ras serves as a molecular switch cycling between inactive (GDP-bound) and active (GTP-bound) states to transduce upstream cellular signals received from multiple tyrosine kinases to downstream effectors to regulate a wide variety of processes, including cellular proliferation. Aberrant expression of K-Ras accounts for up to ˜20% of all cancers and oncogenic K-Ras mutations that stabilize GTP binding and lead to constitutive activation of K-Ras. K-Ras mutations at codons 12, 13, 61 and other positions of the K-Ras primary amino acid sequence are present in 88% of all pancreatic adenocarcinoma patients, 50% of all colorectal adenocarcinoma patients, and 32% lung adenocarcinoma patients. A recent publication also suggested wild type K-Ras inhibition could be a viable therapeutic strategy to treat K-Ras wild type dependent cancers.

Allele-specific K-Ras G12C inhibitors, such as sotorasib (AMG510) or adagrasib (MRTX849), are currently changing the treatment paradigm for patients with K-Ras G12C mutated non-small cell lung cancer and colorectal cancer. The success of addressing a previously elusive K-Ras allele has fueled drug discovery efforts for all K-Ras mutants. Multiple K-Ras inhibitors have the potential to address broad patient populations, including K-Ras G12C, K-Ras G12D, K-Ras G12V, K-Ras G13D, K-Ras G12R, K-Ras G12S, K-Ras G12A, K-Ras Q61H mutant and K-Ras wild type amplified cancers.

Therefore, there are unmet needs to develop new multiple K-Ras inhibitors for treating K-Ras mediated cancers.

### SUMMARY OF THE INVENTION

Provided herein is a compound of Formula (IB), a stereoisomer thereof, a pharmaceutically acceptable salt thereof, a pharmaceutically acceptable salt of the stereoisomer thereof, a prodrug thereof, a deuterated molecule thereof or a conjugated form thereof:

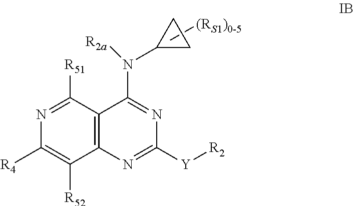

Wherein, the definition of each of variables is as below.

Also provided herein is a pharmaceutical composition comprising a therapeutically effective amount of a compound of Formula (IB), a stereoisomer thereof, a pharmaceutically acceptable salt thereof, a pharmaceutically acceptable salt of the stereoisomer thereof, a prodrug thereof, a deuterated molecule thereof or a conjugated form thereof as defined herein; and a pharmaceutically acceptable excipient.

Also provided herein is a method for treating cancer in a subject comprising administering a therapeutically effective amount of a compound of Formula (IB), a stereoisomer thereof, a pharmaceutically acceptable salt thereof, a pharmaceutically acceptable salt of the stereoisomer thereof, a prodrug thereof, a deuterated molecule thereof, a conjugated form thereof, or a pharmaceutical composition as defined herein to a subject in need thereof.

Also provided herein is a method for treating cancer in a subject in need thereof, the method comprising (a) determining whether the cancer is associated with K-Ras G12C, K-Ras G12D, K-Ras G12V, K-Ras G13D, K-Ras G12R, K-Ras G12S, K-Ras G12A, K-Ras Q61H mutation and/or K-Ras wild type amplification; and (b) if so, administering a therapeutically effective amount of a compound of Formula (IB), a stereoisomer thereof, a pharmaceutically acceptable salt thereof, a pharmaceutically acceptable salt of the stereoisomer thereof, a prodrug thereof, a deuterated molecule thereof, a conjugated form thereof, or a pharmaceutical composition as defined herein to the subject in need thereof.

Also provided herein is a compound of Formula (IB), a stereoisomer thereof, a pharmaceutically acceptable salt thereof, a pharmaceutically acceptable salt of the stereoisomer thereof, a prodrug thereof, a deuterated molecule thereof, a conjugated form thereof, or a pharmaceutical composition as defined herein for use in therapy.

Also provided herein is a compound of Formula (IB), a stereoisomer thereof, a pharmaceutically acceptable salt thereof, a pharmaceutically acceptable salt of the stereoisomer thereof, a prodrug thereof, a deuterated molecule thereof, a conjugated form thereof, or a pharmaceutical composition as defined herein for use as a medicament.

Also provided herein is a compound of Formula (IB), a stereoisomer thereof, a pharmaceutically acceptable salt thereof, a pharmaceutically acceptable salt of the stereoisomer thereof, a prodrug thereof, a deuterated molecule thereof, a conjugated form thereof, or a pharmaceutical composition as defined herein for use in a method for the treatment of cancer.

Also provided herein is a use of a compound of Formula (IB), a stereoisomer thereof, a pharmaceutically acceptable salt thereof, a pharmaceutically acceptable salt of the stereoisomer thereof, a prodrug thereof, a deuterated molecule thereof, a conjugated form thereof, or a pharmaceutical composition as defined herein for the treatment of cancer.

Also provided herein is a use of a compound of Formula (IB), a stereoisomer thereof, a pharmaceutically acceptable salt thereof, a pharmaceutically acceptable salt of the stereoisomer thereof, a prodrug thereof, a deuterated molecule thereof, a conjugated form thereof, or a pharmaceutical composition as defined herein for the manufacture of a medicament for the treatment of cancer.

Also provided herein is a process for preparing a compound of Formula (IB) as defined herein.

Also provided herein is an intermediate for preparing a compound of Formula (IB) as defined herein.

### DETAILED DESCRIPTION

Provided herein are the following disclosures:

[1]. A compound of Formula (IB), a stereoisomer thereof, a pharmaceutically acceptable salt thereof, a pharmaceutically acceptable salt of the stereoisomer thereof, a prodrug thereof, a deuterated molecule thereof or a conjugated form thereof:

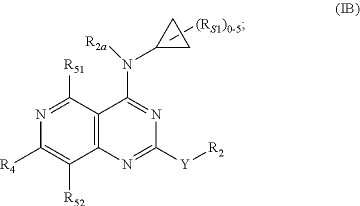

### Wherein,

R₂ₐ is selected from hydrogen, deuterium, —C₁₋₁₀alkyl, haloC₁₋₁₀alkyl, haloC₁₋₁₀alkoxy, —C₂₋₁₀alkenyl, haloC₂₋₁₀alkenyl, —C₂₋₁₀alkynyl, haloC₂₋₁₀alkynyl, —N(R_{b})₂, —OR_{b}, —SR_{b}, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₁₀alkyl, haloC₁₋₁₀alkyl, haloC₁₋₁₀alkoxy, —C₂₋₁₀alkenyl, —C₂₋₁₀alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is optionally independently substituted with one or more substituents selected from deuterium, halogen, —C₁₋₆alkyl, haloC₁₋₆alkyl, haloC₁₋₆alkoxy, —C₂₋₆alkenyl, —C₂₋₆alkynyl, —CN, —NO₂, —N₃, oxo, —N(R_{c})₂, —OR, —SR_{c}, —S(═O)R_{d}, —S(═O)₂Rₐ, —C(═O)R_{d}, —C(═O)OR_{c}, —OC(═O)R_{d}, —C(═O)N(R_{c})₂, —NR_{c}C(═O)R_{d}, —OC(═O)OR_{c}, —NR_{c}C(═O)OR_{d}, —OC(═O)N(R_{c})₂, —NR_{c}C(═O)N(R_{c})₂, —S(═O)OR_{c}, —OS(═O)R_{d}, —S(═O)N(R_{c})₂, —NR_{c}S(═O)R_{d}, —S(═O)₂OR_{c}, —OS(═O)₂Rₐ, —S(═O)₂N(R_{c})₂, —NR_{c}S(═O)₂Rₐ, —OS(═O)₂OR_{c}, —NR_{c}S(═O)₂OR_{c}, —OS(═O)₂NR_{c}, —NR_{c}S(═O)₂N(R_{c})₂, —P(R_{c})₂, —P(═O)(R_{d})₂, 3-6 membered cycloalkyl, 3-6 membered cycloalkenyl, 3-6 membered cycloalkynyl, 3-10 membered heterocyclyl, 6-10 membered aryl or 5-10 membered heteroaryl;
R_{S1} at each occurrence is independently selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R₆₁)₂, —OR₆₁, —SR₆₁, —S(═O)R₆₂, —S(═O)₂R₆₂, —C(═O)R₆₂, —C(═O)OR₆₁, —OC(═O)R₆₂, —C(═O)N(R₆₁)₂, —NR₆₁C(═O)R₆₂, —OC(═O)OR₆₁, —NR₆₁C(═O)OR₆₁, —NR₆₁C(═S)OR₆₁, —OC(═O)N(R₆₁)₂, —NR₆₁C(═O)N(R₆₁)₂, —S(═O)OR₆₁, —OS(═O)R₆₂, —S(═O)N(R₆₁)₂, —NR₆₁S(═O)R₆₂, —S(═O)₂OR₆₁, —OS(═O)₂R₆₂, —S(═O)₂N(R₆₁)₂, —NR₆₁S(═O)₂R₆₂, —OS(═O)₂OR₆₁, —NR₆₁S(═O)₂OR₆₁, —OS(═O)₂N(R₆₁)₂, —NR₆₁S(═O)₂N(R₆₁)₂, —P(R₆₁)₂, —P(═O)(R₆₂)₂,

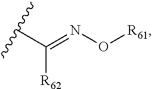

3-8 membered cycloalkyl, 3-8 membered cycloalkenyl, 3-8 membered cycloalkynyl, 4-8 membered heterocyclyl, 6-10 membered aryl, 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, 3-8 membered cycloalkyl, 3-8 membered cycloalkenyl, 3-8 membered cycloalkynyl, 3-8 membered heterocyclyl, 6-10 membered aryl or 5-10 membered heteroaryl is optionally independently substituted with one or more substituents selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R₆₃)₂, —OR₆₃, —SR₆₃, —S(═O)R₆₄, —S(═O)₂R₆₄, —C(═O)R₆₄, —C(═O)OR₆₄, —OC(═O)R₆₄, —C(═O)N(R₆₃)₂, —NR₆₃C(═O)R₆₄, —OC(═O)OR₆₃, —NR₆₃C(═O)OR₆₃, —NR₆₃C(═S)OR₆₃, —OC(═O)N(R₆₃)₂, —NR₆₃C(═O)N(R₆₃)₂, —S(═O)OR₆₃, —OS(═O)R₆₄, —S(═O)N(R₆₃)₂, —NR₆₃S(═O)R₆₄, —S(═O)₂OR₆₃, —OS(═O)₂R₆₄, —S(═O)₂N(R₆₃)₂, —NR₆₃S(═O)₂R₆₄, —OS(═O)₂OR₆₃, —NR₆₃S(═O)₂OR₆₃, —OS(═O)₂N(R₆₃)₂, —NR₆₃S(═O)₂N(R₆₃)₂, —P(R₆₃)₂, —P(═O)(R₆₄)₂, 3-6 membered cycloalkyl, 3-6 membered cycloalkenyl, 3-6 membered cycloalkynyl, 3-6 membered heterocyclyl, 6-10 membered aryl or 5-10 membered heteroaryl;
Optionally, two R_{S1} together with the carbon atom to which they are both attached form

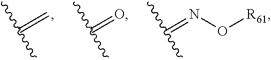

a 3-10 membered carbocyclic ring or a 3-10 heterocyclic ring; wherein, said

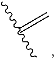

membered carbocylic ring or 3-10 heterocyclic ring is optionally substituted with one or more R_{16c};
Optionally, two adjacent R_{S1} together with the carbon atoms to which they are respectively attached form a 3-10 membered carbocyclic ring, a 3-10 membered heterocyclic ring, a 6-10 membered aryl ring or a 5-10 membered heteroaryl ring, wherein, each of rings is independently optionally substituted with one or more R_{16d};
Optionally, two nonadjacent R_{S1} are connected together to form a bridge containing 0, 1, 2, 3, 4, 5 or 6 carbon atoms, wherein, each of the carbon atoms in the bridge is optionally replaced by 1 or 2 heteroatoms selected from N, O, S, S═O or S(═O)₂; the hydrogen on the each of carbon atoms or N atoms is optionally independently substituted with R₁₆ₑ;
Y is a bond, O, S, S(═O), S(═O)₂ or NR₆ₐ;
R₂ is selected from -L₅-(3-12 membered heterocyclyl), -L₅-(3-12 membered cycloalkyl), -L₅-(6-12 member aryl), -L₅-(5-12 membered heteroaryl), -L₅-N(R₇ₐ)₂,

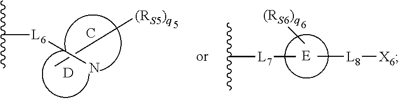

Each of L₅ at each occurrence is independently selected from a bond or C₁₋₁₀ alkylene optionally substituted with one or more R₁₆;
Said 3-12 membered heterocyclyl in -L₅-(3-12 membered heterocyclyl) is optionally substituted with one or more R₁₆ₒ;
Said 3-12 membered cycloalkyl in -L₅-(3-12 membered cycloalkyl) is optionally substituted with one or more R₁₆ₒ;
Said 6-12 member aryl in -L₅-(6-12 member aryl) is optionally substituted with one or more R₁₆ₒ;
Said 5-12 membered heteroaryl in -L₅-(5-12 membered heteroaryl) is optionally substituted with one or more R₁₆ₒ;
L₆ is selected from a bond or C₁₋₁₀ alkylene optionally substituted with one or more R₁₆ₚ;
L₇ is selected from a bond or C₁₋₁₀ alkylene optionally substituted with one or more R_{16q};
L₈ is selected from a bond or C₁₋₁₀ alkylene optionally substituted with one or more R₁₆ᵣ;
Ring C or ring D is a 3-10 membered heterocyclic ring which is optionally further contains 1, 2, or 3 heteroatoms selected from N, O or S;
Ring E is selected from a 3-10 membered carbocyclic ring or a 3-10 membered heterocyclic ring; wherein the moiety of -L₇- and -L₈-X₆ are attached to the same atom or different atoms of the ring E;
X₆ is selected from —N(R₆₅)₂, —OR₆₅, —SR₆₅, 3-10 membered heterocyclyl, or 5-10 membered heteroaryl, wherein said 3-10 membered heterocyclyl or 5-10 membered heteroaryl is optionally independently substituted with one or more R₁₆ₛ;
Each of R_{S5} at each occurrence is independently selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R₆₆)₂, —OR₆₆, —SR₆₆, —S(═O)R_{6y}, —S(═O)₂R₆₇, —C(═O)R_{6y}, —C(═O)OR₆₆, —OC(═O)R₆₇, —C(═O)N(R₆₆)₂, —NR₆₆C(═O)R_{6y}, —OC(═O)OR₆₆, —NR₆₆C(═O)OR₆₆, —NR₆₆C(═S)OR₆₆, —OC(═O)N(R₆₆)₂, —NR₆₆C(═O)N(R₆₆)₂, —S(═O)OR₆₆, —OS(═O)R_{6y}, —S(═O)N(R₆₆)₂, —NR₆₆S(═O)R₆₆, —S(═O)₂OR₆₆, —OS(═O)₂R₆₇, —S(═O)₂N(R₆₆)₂, —NR₆₆S(═O)₂R₆₇, —OS(═O)₂OR₆₆, —NR₆₆S(═O)₂OR₆₆, —OS(═O)₂N(R₆₆)₂, —NR₆₆S(═O)₂N(R₆₆)₂, —P(R₆₆)₂, —P(═O)(R₆₇)₂,

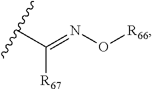

3-8 membered cycloalkyl, 3-8 membered cycloalkenyl, 3-8 membered cycloalkynyl, 4-8 membered heterocyclyl, 6-10 membered aryl, 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, 3-8 membered cycloalkyl, 3-8 membered cycloalkenyl, 3-8 membered cycloalkynyl, 3-8 membered heterocyclyl, 6-10 membered aryl or 5-10 membered heteroaryl is optionally independently substituted with one or more substituents selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R₆₈)₂, —OR₆₈, —SR₆₈, —S(═O)R₆₉, —S(═O)₂R₆₉, —C(═O)R₆₉, —C(═O)OR₆₈, —OC(═O)R₆₉, —C(═O)N(R₆₈)₂, —NR₆₈C(═O)R₆₉, —OC(═O)OR₆₈, —NR₆₈C(═O)OR₆₈, —NR₆₈C(═S)OR₆₈, —OC(═O)N(R₆₈)₂, —NR₆₈C(═O)N(R₆₈)₂, —S(═O)OR₆₈, —OS(═O)R₆₉, —S(═O)N(R₆₈)₂, —NR₆₈S(═O)R₆₉, —S(═O)₂OR₆₈, —OS(═O)₂R₆₉, —S(═O)₂N(R₆₈)₂, —NR₆₈S(═O)₂R₆₉, —OS(═O)₂OR₆₈, —NR₆₈S(═O)₂OR₆₈, —OS(═O)₂N(R₆₈)₂, —NR₆₈S(═O)₂N(R₆₈)₂, —P(R₆₈)₂, —P(═O)(R₆₉)₂, 3-6 membered cycloalkyl, 3-6 membered cycloalkenyl, 3-6 membered cycloalkynyl, 3-6 membered heterocyclyl, 6-10 membered aryl or 5-10 membered heteroaryl;
Optionally, two R_{S5} together with the carbon atom to which they are both attached form

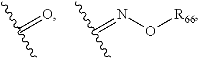

a 3-10 membered carbocyclic ring or a 3-10 heterocyclic ring; wherein, said 3-10 membered carbocyclic ring or 3-10 heterocyclic ring is optionally substituted with one or more R₁₆ₜ;
Optionally, two adjacent R_{S5} together with the carbon atoms to which they are respectively attached form a 3-10 membered carbocyclic ring, a 3-10 membered heterocyclic ring, a 6-10 membered aryl ring or a 5-10 membered heteroaryl ring, wherein, each of rings is independently optionally substituted with one or more R₁₆ᵤ;
Optionally, two nonadjacent R_{S5} are connected together to form a bridge containing 0, 1, 2, 3, 4, 5 or 6 carbon atoms, wherein, each of the carbon atoms in the bridge is optionally replaced by 1 or 2 heteroatoms selected from N, O, S, S═O or S(═O)₂; the hydrogen on the each of carbon atoms or N atoms is optionally independently substituted with R₁₆ᵥ;
q₅ is selected from 0, 1, 2, 3, 4, 5 or 6;
Each of R_{S6} at each occurrence is independently selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R₇₁)₂, —OR₇₁, —SR₇₁, —S(═O)R₇₂, —S(═O)₂R₇₁, —C(═O)R₇₂, —C(═O)OR₇₁, —OC(═O)R₇₂, —C(═O)N(R₇₁)₂, —NR₇₁C(═O)R₇₂, —OC(═O)OR₇₁, —NR₇₁C(═O)OR₇₁, —OC(═O)N(R₇₁)₂, —NR₇₁C(═O)N(R₇₁)₂, —S(═O)OR₇₁, —OS(═O)R₇₂, —S(═O)N(R₇₁)₂, —NR₇₁S(═O)R₇₂, —S(═O)₂OR₇₁, —OS(═O)₂R₇₂, —S(═O)₂N(R₇₁)₂, —NR₇₁S(═O)₂R₇₂, —OS(═O)₂OR₇₁, —NR₇₁S(═O)₂OR₇₂, —OS(═O)₂N(R₇₁)₂, —NR₇₁S(═O)₂N(R₇₁)₂, —P(R₇₁)₂, —P(═O)(R₇₂)₂, 3-6 membered cycloalkyl, 3-6 membered cycloalkenyl, 3-6 membered cycloalkynyl, 3-6 membered heterocyclyl, 6-10 membered aryl or 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, 3-6 membered cycloalkyl, 3-6 membered cycloalkenyl, 3-6 membered cycloalkynyl, 3-6 membered heterocyclyl, 6-10 membered aryl or 5-10 membered heteroaryl is optionally independently substituted with one or more substituents selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R₇₃)₂, —OR₇₃, —SR₇₃, —S(═O)R₇₄, —S(═O)₂R₇₃, —C(═O)R₇₄, —C(═O)OR₇₃, —OC(═O)R₇₄, —C(═O)N(R₇₃)₂, —NR₇₃C(═O)R₇₄, —OC(═O)OR₇₃, —NR₇₃C(═O)OR₇₃, —OC(═O)N(R₇₃)₂, —NR₇₃C(═O)N(R₇₃)₂, —S(═O)OR₇₃, —OS(═O)R₇₄, —S(═O)N(R₇₃)₂, —NR₇₃S(═O)R₇₄, —S(═O)₂OR₇₃, —OS(═O)₂R₇₄, —S(═O)₂N(R₇₃)₂, —NR₇₃S(═O)₂R₇₄, —OS(═O)₂OR₇₃, —NR₇₃S(═O)₂OR₇₄, —OS(═O)₂N(R₇₃)₂, —NR₇₃S(═O)₂N(R₇₃)₂, —P(R₇₃)₂, —P(═O)(R₇₄)₂, 3-6 membered cycloalkyl, 3-6 membered cycloalkenyl, 3-6 membered cycloalkynyl, 3-6 membered heterocyclyl, 6-10 membered aryl or 5-10 membered heteroaryl;
q₆ is selected from 0, 1, 2, 3, 4, 5 or 6;
R₄ is selected from 6-10 membered aryl, 5-10 membered heteroaryl

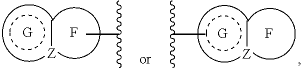

wherein said 6-10 membered aryl, 5-10 membered heteroaryl,

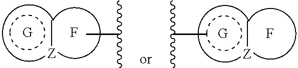

is optionally independently substituted with one or more R₄₁;
Z at each occurrence is independently selected from C or N;
Ring G at each occurrence is independently selected from a 6 membered aryl ring or a 5-6 membered heteroaryl ring and ring F at each occurrence is a 3-10 membered carbocyclic ring or a 3-10 membered heterocyclic ring when Z is selected from C;
Ring G at each occurrence is selected from a 5-6 membered heteroaryl ring and ring F at each occurrence is a 3-10 membered heterocyclic ring when Z is selected from N;
R₄₁ at each occurrence is independently selected from deuterium, halogen, —C₁₋₁₀alkyl, haloC₁₋₁₀alkyl, haloC₁₋₁₀alkoxy, —C₂₋₁₀alkenyl, haloC₂₋₁₀alkenyl, —C₂₋₁₀alkynyl, haloC₂₋₁₀alkynyl, —CN, —NO₂, —N₃, oxo, —N(R₇₅)₂, —OR₇₅, —SR₇₅, —S(═O)R₇₆, —S(═O)₂R₇₆, —C(═O)R₇₆, —C(═O)OR₇₅, —OC(═O)R₇₆, —C(═O)N(R₇₅)₂, —NR₇₅C(═O)R₇₆, —OC(═O)OR₇₅, —NR₇₅C(═O)OR₇₅, —OC(═O)N(R₇₅)₂, —NR₇₅C(═O)N(R₇₅)₂, —S(═O)OR₇₅, —OS(═O)R₇₆, —S(═O)N(R₇₅)₂, —NR₇₅S(═O)R₇₆, —S(═O)₂OR₇₅, —OS(═O)₂R₇₆, —S(═O)₂N(R₇₅)₂, —NR₇₅S(═O)₂R₇₆, —OS(═O)₂OR₇₅, —NR₇₅S(═O)₂OR₇₅, —OS(═O)₂N(R₇₅)₂, —NR₇₅S(═O)₂N(R₇₅)₂, —P(R₇₅)₂, —P(═O)(R₇₅)₂, 3-10 membered cycloalkyl, 3-10 membered cycloalkenyl, 3-10 membered cycloalkynyl, 3-10 membered heterocyclyl, 6-10 membered aryl or 5-10 membered heteroaryl, wherein said —C₁₋₁₀alkyl, haloC₁₋₁₀alkyl, haloC₁₋₁₀alkoxy, —C₂₋₁₀alkenyl, haloC₂₋₁₀alkenyl, —C₂₋₁₀alkynyl, haloC₂₋₁₀alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is optionally independently substituted with one or more substituents selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R₇₇)₂, —OR₇₇, —SR₇₇, —S(═O)R₇₈, —S(═O)₂R₇₈, —C(═O)R₇₈, —C(═O)OR₇₇, —OC(═O)R₇₈, —C(═O)N(R₇₇)₂, —NR₇₇C(═O)R₇₈, —OC(═O)OR₇₇, —NR₇₇C(═O)OR₇₇, —OC(═O)N(R₇₇)₂, —NR₇₇C(═O)N(R₇₇)₂, —S(═O)OR₇₇, —OS(═O)Rys, —S(═O)N(R₇₇)₂, —NR₇₇S(═O)R₇₈, —S(═O)₂OR₇₇, —OS(═O)₂R₇₈, —S(═O)₂N(R₇₇)₂, —NR₇₇S(═O)₂R₇₈, —OS(═O)₂OR₇₇, —NR₇₇S(═O)₂OR₇₇, —OS(═O)₂N(R₇₇)₂, —NR₇₇S(═O)₂N(R₇₇)₂, —P(R₇₇)₂, —P(═O)(R₇₈)₂, 3-6 membered cycloalkyl, 3-6 membered cycloalkenyl, 3-6 membered cycloalkynyl, 3-6 membered heterocyclyl, 6-10 membered aryl or 5-10 membered heteroaryl;
Each of (R₅₁ and R₅₂) is independently selected from hydrogen, deuterium, halogen, —C₁₋₁₀alkyl, haloC₁₋₁₀alkyl, haloC₁₋₁₀alkoxy, —C₂₋₁₀alkenyl, haloC₂₋₁₀alkenyl, —C₂₋₁₀alkynyl, haloC₂₋₁₀alkynyl, —CN, —NO₂, —N₃, oxo, —N(R₈₁)₂, —OR₈₁, —SR₁, —S(═O)R₈₂, —S(═O)₂R₈₂, —C(═O)R₈₂, —C(═O)OR₈₁, —OC(═O)R₈₂, —C(═O)N(R₈₁)₂, —NR₈₁C(═O)R₈₂, —OC(═O)OR₈₁, —NR₈₁C(═O)OR₈₁, —OC(═O)N(R %1)₂, —NR₈₁C(═O)N(R₈₁)₂, —S(═O)OR₈₁, —OS(═O)R₈₂, —S(═O)N(R₈₁)₂, —NR₈₁S(═O)R₈₂, —S(═O)₂OR₈₁, —OS(═O)₂R₈₂, —S(═O)₂N(R₈₁)₂, —NR₈₁S(═O)₂R₈₂, —OS(═O)₂OR₈₁, —NR₈₁S(═O)₂OR₈₁, —OS(═O)₂N(R₈₁)₂, —NR₈₁S(═O)₂N(R₈₁)₂, —P(R₈₁)₂, —P(═O)(R₈₂)₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₁₀alkyl, haloC₁₋₁₀alkyl, haloC₁₋₁₀alkoxy, —C₂₋₁₀alkenyl, haloC₂₋₁₀alkenyl, —C₂₋₁₀alkynyl, haloC₂₋₁₀alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is optionally independently substituted with one or more substituents selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R₈₃)₂, —OR₈₃, —SR₈₃, —S(═O)R₈₄, —S(═O)₂R₈₄, —C(═O)R₈₄, —C(═O)OR₈₃, —OC(═O)R₈₃, —C(═O)N(R₈₃)₂, —NR₈₃C(═O)R₈₄, —OC(═O)OR₈₃, —NR₈₃C(═O)OR₈₃, —OC(═O)N(R₈₃)₂, —NR₈₃C(═O)N(R₈₄)₂, —S(═O)OR₈₃, —OS(═O)R₈₄, —S(═O)N(R₈₃)₂, —NR₈₃S(═O)R %₄, —S(═O)₂OR₈₃, —OS(═O)₂R₈₄, —S(═O)₂N(R₈₃)₂, —NR₈₃S(═O)₂R₈₄, —OS(═O)₂OR₈₃, —NR₈₃S(═O)₂OR₈₃, —OS(═O)₂N(R₈₃)₂, —NR₈₃S(═O)₂N(R₈₃)₂, —P(R₈₃)₂, —P(═O)(R₈₄)₂, 3-6 membered cycloalkyl, 3-6 membered cycloalkenyl, 3-6 membered cycloalkynyl, 3-6 membered heterocyclyl, 6-10 membered aryl or 5-10 membered heteroaryl;
Each of (R₆ₐ, R₇ₐ, R₆₁, R₆₃, R₆₅, R₆₆, R₆₈, R₇₁, R₇₃, R₇₅, R₇₇, R₈₁ and R₈₃) at each occurrence is independently selected from hydrogen, deuterium, halogen, —C₁₋₁₀alkyl, haloC₁₋₁₀alkyl, —C₂₋₁₀alkenyl, —C₂₋₁₀alkynyl, —S(═O)Rₐ, —S(═O)₂Rₐ, —C(═O)Rₐ, —C(═O)ORₐ, —C(═O)N(Rₐ)₂, —S(═O)ORₐ, —S(═O)N(Rₐ)₂, —S(═O)₂ORₐ, —S(═O)₂N(Rₐ)₂, —P(═O)(Rₐ)₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₁₀alkyl, haloC₁₋₁₀alkyl, —C₂₋₁₀alkenyl, —C₂₋₁₀alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is optionally independently substituted with one or more substituents selected from deuterium, halogen, —C₁₋₆alkyl, haloC₁₋₆alkyl, haloC₁₋₆alkoxy, —C₂₋₆alkenyl, —C₂₋₆alkynyl, —CN, —NO₂, —N₃, oxo, —N(R_{c})₂, —OR_{c}, —SR_{c}, —S(═O)R_{d}, —S(═O)₂R_{d}, —C(═O)R_{d}, —C(═O)OR, —OC(═O)R_{d}, —C(═O)N(R)₂, —NRC(═O)Rₐ, —OC(═O)OR, —NRC(═O)ORₐ, —OC(═O)N(R)₂, —NRC(═O)N(R_{c})₂, —S(═O)OR_{c}, —OS(═O)R_{d}, —S(═O)N(R)₂, —NRS(═O)Rₐ, —S(═O)₂OR, —OS(═O)₂R_{d}, —S(═O)₂N(R)₂, —NRS(═O)₂R_{d}, —OS(═O)₂OR, —NRS(═O)₂OR, —OS(═O)₂NR, —NRS(═O)₂N(R)₂, —P(R_{c})₂, —P(═O)(Rₐ)₂, 3-6 membered cycloalkyl, 3-6 membered cycloalkenyl, 3-6 membered cycloalkynyl, 3-6 membered heterocyclyl, 6-10 membered aryl or 5-10 membered heteroaryl;
Optionally, each of (two R₇ₐ, two R₆₁, two R₆₃, two R₆₅, two R₆₆, two R₆₈, two R₇₁, two R₇₃, two R₇₅, two R₇₇, two R₈₁, and two R₈₃) independently together with the nitrogen atom to which they are both attached forms a 3-20 membered heterocyclic ring or a 5-10 membered heteroaryl ring, wherein, said 3-20 membered heterocyclic ring or 5-10 membered heteroaryl ring is optionally independently substituted with one or more R_{16W};
Each of (R₆₂, R₆₄, R₆₇, R₆₉, R₇₂, R₇₄, R₇₆, R₇₈, R₈₂ and R₈₄) at each occurrence is independently selected from hydrogen, deuterium, —C₁₋₁₀alkyl, haloC₁₋₁₀alkyl, haloC₁₋₁₀alkoxy, —C₂₋₁₀alkenyl, haloC₂₋₁₀alkenyl, —C₂₋₁₀alkynyl, haloC₂₋₁₀alkynyl, —N(R_{b})₂, —OR_{b}, —SR_{b}, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₁₀alkyl, haloC₁₋₁₀alkyl, haloC₁₋₁₀alkoxy, —C₂₋₁₀alkenyl, —C₂₋₁₀alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is optionally independently substituted with one or more substituents selected from deuterium, halogen, —C₁₋₆alkyl, haloC₁₋₆alkyl, haloC₁₋₆alkoxy, —C₂₋₆alkenyl, —C₂₋₆alkynyl, —CN, —NO₂, —N₃, oxo, —N(R_{c})₂, —OR, —SR, —S(═O)Rₐ, —S(═O)₂Rₐ, —C(═O)Rₐ, —C(═O)OR, —OC(═O)R_{d}, —C(═O)N(R)₂, —NRC(═O)Rₐ, —OC(═O)OR, —NRC(═O)ORₐ, —OC(═O)N(R)₂, —NRC(═O)N(R)₂, —S(═O)OR, —OS(═O)R_{d}, —S(═O)N(R)₂, —NRS(═O)Rₐ, —S(═O)₂OR, —OS(═O)₂R_{d}, —S(═O)₂N(R)₂, —NRS(═O)₂R_{d}, —OS(═O)₂OR, —NRS(═O)₂OR, —OS(═O)₂NR, —NRS(═O)₂N(R_{c})₂, —P(R_{c})₂, —P(═O)(R_{d})₂, 3-6 membered cycloalkyl, 3-6 membered cycloalkenyl, 3-6 membered cycloalkynyl, 3-10 membered heterocyclyl, 6-10 membered aryl or 5-10 membered heteroaryl;
Each of (Rₐ, R_{b}, R_{c} and R_{d}) at each occurrence is independently selected from hydrogen, deuterium, —C₁₋₆alkyl, haloC₁₋₆alkyl, haloC₁₋₆alkoxy, —C₂₋₆alkenyl, —C₂₋₆alkynyl, 3-6 membered cycloalkyl, 3-6 membered cycloalkenyl, 3-6 membered cycloalkynyl, 3-6 membered heterocyclyl, 6-10 membered aryl or 5-10 membered heteroaryl; wherein said —C₁₋₆alkyl, haloC₁₋₆alkyl, haloC₁₋₆alkoxy, —C₂₋₆alkenyl, —C₂₋₆alkynyl, 3-6 membered cycloalkyl, 3-6 membered cycloalkenyl, 3-6 membered cycloalkynyl, 3-6 membered heterocyclyl, 6-10 membered aryl or 5-10 membered heteroaryl is optionally independently substituted with one or more R₁₆ₓ;
Optionally, each of (two Rₐ, two R_{b} and two R_{c}) independently together with the atom to which they are both attached forms a 3-6 membered heterocyclic ring, wherein said 3-6 membered heterocyclic ring is independently optionally substituted with one or more R_{16y};
Each of (R_{16c}, R_{16d}, R₁₆ₑ, R₁₆ₙ, R₁₆ₒ, R₁₆ₚ, R_{16q}, R₁₆ᵣ, R₁₆ₛ, R₁₆ₜ, R₁₆ᵤ, R₁₆ᵥ, R_{16w}, R₁₆ₓ and R_{16y}) at each occurrence is independently selected from deuterium, halogen, —C₁₋₆alkyl, haloC₁₋₆alkyl, haloC₁₋₆alkoxy, —C₂₋₆alkenyl, —C₂₋₆alkynyl, —CN, —NO₂, —N₃, oxo, —NH₂, —NH(C₁₋₆alkyl), —N(C₁₋₆alkyl)₂, —OH, —O(C₁₋₆alkyl), —SH, —S(C₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —OC(═O)O(C₁₋₆alkyl), —NHC(═O)(OC₁₋₆alkyl), —N(C₁₋₆alkyl)C(═O)(OC₁₋₆alkyl), —OC(═O)NH(C₁₋₆alkyl), —OC(═O)N(C₁₋₆alkyl)₂, —NHC(═O)NH₂, —NHC(═O)NH(C₁₋₆alkyl), —NHC(═O)N(C₁₋₆alkyl)₂, —N(C₁₋₆alkyl)C(═O)NH₂, —N(C₁₋₆alkyl)C(═O)NH(C₁₋₆alkyl), —N(C₁₋₆alkyl)C(═O)N(C₁₋₆alkyl)₂, —S(═O)(OC₁₋₆alkyl), —OS(═O)(C₁₋₆alkyl), —S(═O)NH₂, —S(═O)NH(C₁₋₆alkyl), —S(═O)N(C₁₋₆alkyl)₂, —NHS(═O)(C₁₋₆alkyl), —N(C₁₋₆alkyl)S(═O)(C₁₋₆alkyl), —S(═O)₂(OC₁₋₆alkyl), —OS(═O)₂(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), —OS(═O)₂O(C₁₋₆alkyl), —NHS(═O)₂O(C₁₋₆alkyl), —N(C₁₋₆alkyl)S(═O)₂O(C₁₋₆alkyl), —OS(═O)₂NH₂, —OS(═O)₂NH(C₁₋₆alkyl), —OS(═O)₂N(C₁₋₆alkyl)₂, —NHS(═O)₂NH₂, —NHS(═O)₂NH(C₁₋₆alkyl), —NHS(═O)₂N(C₁₋₆alkyl)₂, —N(C₁₋₆alkyl)S(═O)₂NH₂, —N(C₁₋₆alkyl)S(═O)₂NH(C₁₋₆alkyl), —N(C₁₋₆alkyl)S(═O)₂N(C₁₋₆alkyl)₂, —PH(C₁₋₆alkyl), —P(C₁₋₆alkyl)₂, —P(═O)H(C₁₋₆alkyl), —P(═O)(C₁₋₆alkyl)₂, 3-6 membered cycloalkyl, 3-6 membered cycloalkenyl, 3-6 membered cycloalkynyl, 3-6 membered heterocyclyl, 6-10 membered aryl or 5-10 membered heteroaryl; wherein, said —C₁₋₆alkyl, haloC₁₋₆alkyl, haloC₁₋₆alkoxy, —C₂₋₆alkenyl, —C₂₋₆alkynyl, 3-6 membered cycloalkyl, 3-6 membered cycloalkenyl, 3-6 membered cycloalkynyl, 3-6 membered heterocyclyl, 6-10 membered aryl or 5-10 membered heteroaryl is optionally substituted with one or more substituents selected from deuterium, halogen, —C₁₋₃alkyl, haloC₁₋₃alkyl, haloC₁₋₃alkoxy, —C₂₋₃alkenyl, —C₂₋₃alkynyl, —CN, —NO₂, —N₃, oxo, —NH₂, —NH(C₁₋₃alkyl), —N(C₁₋₃alkyl)₂, —OH, —O(C₁₋₃alkyl), —SH, —S(C₁₋₃alkyl), —S(═O)(C₁₋₃alkyl), —S(═O)₂(C₁₋₃alkyl), —C(═O)(C₁₋₃alkyl), —C(═O)OH, —C(═O)(OC₁₋₃alkyl), —OC(═O)(C₁₋₃alkyl), —C(═O)NH₂, —C(═O)NH(C₁₋₃alkyl), —C(═O)N(C₁₋₃alkyl)₂, —NHC(═O)(C₁₋₃alkyl), —N(C₁₋₃alkyl)C(═O)(C₁₋₃alkyl), —OC(═O)O(C₁₋₃alkyl), —NHC(═O)(OC₁₋₃alkyl), —N(C₁₋₃alkyl)C(═O)(OC₁₋₃alkyl), —OC(═O)NH(C₁₋₃alkyl), —OC(═O)N(C₁₋₃alkyl)₂, —NHC(═O)NH₂, —NHC(═O)NH(C₁₋₃alkyl), —NHC(═O)N(C₁₋₃alkyl)₂, —N(C₁₋₃alkyl)C(═O)NH₂, —N(C₁₋₃alkyl)C(═O)NH(C₁₋₃alkyl), —N(C₁₋₃alkyl)C(═O)N(C₁₋₃alkyl)₂, —S(═O)(OC₁₋₃alkyl), —OS(═O)(C₁₋₃alkyl), —S(═O)NH₂, —S(═O)NH(C₁₋₃alkyl), —S(═O)N(C₁₋₃alkyl)₂, —NHS(═O)(C₁₋₃alkyl), —N(C₁₋₃alkyl)S(═O)(C₁₋₃alkyl), —S(═O)₂(OC₁₋₃alkyl), —OS(═O)₂(C₁₋₃alkyl), —S(═O)₂NH₂, —S(═O)₂NH(C₁₋₃alkyl), —S(═O)₂N(C₁₋₃alkyl)₂, —NHS(═O)₂(C₁₋₃alkyl), —N(C₁₋₃alkyl)S(═O)₂(C₁₋₃alkyl), —OS(═O)₂O(C₁₋₃alkyl), —NHS(═O)₂O(C₁₋₃alkyl), —N(C₁₋₃alkyl)S(═O)₂O(C₁₋₃alkyl), —OS(═O)₂NH₂, —OS(═O)₂NH(C₁₋₃alkyl), —OS(═O)₂N(C₁₋₃alkyl)₂, —NHS(═O)₂NH₂, —NHS(═O)₂NH(C₁₋₃alkyl), —NHS(═O)₂N(C₁₋₃alkyl)₂, —N(C₁₋₃alkyl)S(═O)₂NH₂, —N(C₁₋₃alkyl)S(═O)₂NH(C₁₋₃alkyl), —N(C₁₋₃alkyl)S(═O)₂N(C₁₋₃alkyl)₂, —PH(C₁₋₃alkyl), —P(C₁₋₃alkyl)₂, —P(═O)H(C₁₋₃alkyl), —P(═O)(C₁₋₃alkyl)₂, 3-6 membered cycloalkyl, 3-6 membered cycloalkenyl, 3-6 membered cycloalkynyl, 3-6 membered heterocyclyl, 6 membered aryl or 5-6 membered heteroaryl;
Each of (heterocyclyl and heteroaryl) at each occurrence is independently contain 1, 2, 3 or 4 heteroatoms selected from N, O, S, S(═O) or S(═O)₂.

[2]. The compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of [1], wherein, the compound is selected from any one of the following formulas:

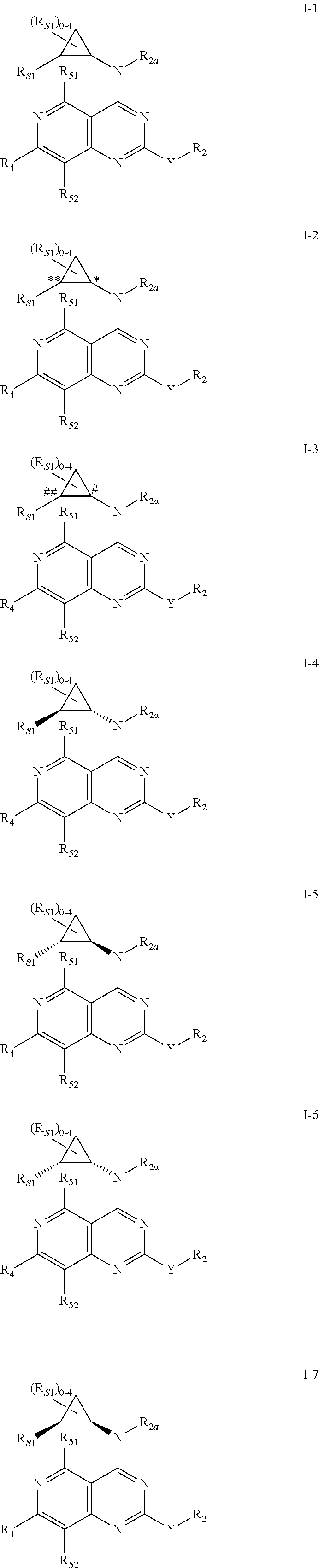

Wherein:
R_{S1} linked with the carbon atom indicated with ** and NR₂ₐ linked with the carbon atom indicated with * are in a trans configuration;
R_{S1} linked with the carbon atom indicated with ## and NR₂ₐ linked with the carbon atom indicated with # are in a cis configuration.

[3]. The compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of [1] or [2], wherein, R₂ₐ is selected from hydrogen, deuterium, —C₁₋₆alkyl, haloC₁₋₆alkyl, —C₁₋₆alkoxy, —C(═O)C₁₋₆alkyl, 3-6 membered cycloalkyl, 3-6 membered heterocyclyl containing 1 or 2 heteroatoms selected from N, O, S, S(═O) or S(═O)₂, phenyl, or 5-6 membered heteroaryl containing 1 or 2 heteroatoms selected from N, O or S; wherein said —C₁₋₆alkyl, haloC₁₋₆alkyl, —C₁₋₆alkoxy, —C(═O)C₁₋₆alkyl, 3-6 membered cycloalkyl, 3-6 membered heterocyclyl, phenyl or 5-6 membered heteroaryl is optionally independently substituted with one or more substituents selected from deuterium, halogen, —C₁₋₃alkyl, haloC₁₋₃alkyl, —C₂₋₃alkenyl, —C₂₋₃alkynyl, —CN, —NH₂, —NH(C₁₋₃alkyl), —N(C₁₋₃alkyl)₂, —OH, —OC₁₋₃alkyl, —SH, —SC₁₋₃alkyl, —C(═O)C₁₋₃alkyl, —C(═O)OH, —C(═O)OC₁₋₃alkyl, —OC(═O)C₁₋₃alkyl, 3-6 membered cycloalkyl or 3-6 membered heterocyclyl containing 1 or 2 heteroatoms selected from N, O, S, S(═O) or S(═O)₂, phenyl, or 5-6 membered heteroaryl containing 1 or 2 heteroatoms selected from N, O or S.

[4]. The compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of any one of [1] to [3], wherein, R₂ₐ is selected from hydrogen, deuterium, methyl, ethyl, propyl, isopropyl, n-butyl, isobutyl, sec-butyl, tert-butyl, n-pentyl, isopentyl, sec-pentyl, tert-pentyl, n-hexyl, iso-hexyl, sec-hexyl, tert-hexyl, halomethyl, haloethyl, methoxy, ethoxy, —C(═O)CH₃, —C(═O)CH₂CH₃, cyclopropyl, cyclobutyl, cyclopentyl, cyclohexyl, pyrrolidinyl, tetrahydrofuryl, tetrahydropyryl, phenyl, thiophenyl or pyridinyl, wherein, said methyl, ethyl, propyl, isopropyl, n-butyl, isobutyl, sec-butyl, tert-butyl, n-pentyl, isopentyl, sec-pentyl, tert-pentyl, n-hexyl, iso-hexyl, sec-hexyl, tert-hexyl, halomethyl, haloethyl, methoxy, ethoxy, —C(═O)CH₃, —C(═O)CH₂CH₃, cyclopropyl, cyclobutyl, cyclopentyl, cyclohexyl, pyrrolidinyl, tetrahydrofuryl, tetrahydropyryl, phenyl, thiophenyl or pyridinyl is optionally independently substituted with 1, 2, 3, 4, 5 or 6 substituents selected from deuterium, —F, methyl, ethyl, propyl, isopropyl, —CH₂F, —CHF₂, —CF₃, —CN, —NH₂, —NHCH₃, —N(CH₃)₂, —OH, —OCH₃, —SH, —SCH₃, —C(═O)CH₃, —C(═O)OH, —C(═O)OCH₃, —C(═O)OCH₂CH₃, —OC(═O)CH₃, cyclopropyl, cyclobutyl, cyclopentyl or cyclohexyl.

[5]. The compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of any one of [1] to [4], wherein, R₂ₐ is selected from any moiety in the Table 1:

TABLE 1
—H,
—CH₃,
—CD₃,
—OCH₃,
—CH₂CN,
—CH₂CH₃,
—CH₂CH₂F,
—CH₂CHF₂,
—CH₂CH₂OCH₃,
—C(═O)CH₃,
—CH₂C(═O)OCH₂CH₃,

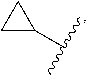 [SMILES: *C1CC1]

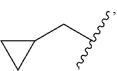 [SMILES: *CC1CC1]

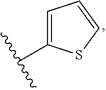 [SMILES: *c1cccs1]

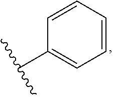 [SMILES: *c1ccccc1]

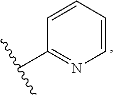 [SMILES: *c1ccccn1]

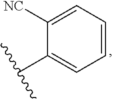 [SMILES: *c1ccccc1C#N]

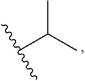 [SMILES: *C(C)C]

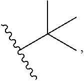 [SMILES: *C(C)(C)C]

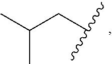 [SMILES: *CC(C)C]

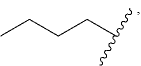 [SMILES: *CCCC]

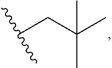 [SMILES: *CC(C)(C)C]

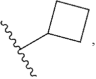 [SMILES: *C1CCC1]

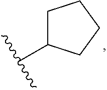 [SMILES: *C1CCCC1]

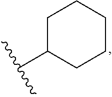 [SMILES: *C1CCCCC1]

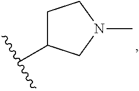 [SMILES: *C1CCN(C)C1]

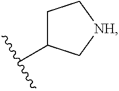 [SMILES: *C1CCNC1]

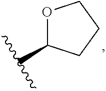 [SMILES: *[C@@H]1CCCO1]

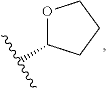 [SMILES: *[C@H]1CCCO1]

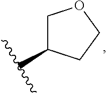 [SMILES: *[C@@H]1CCOC1]

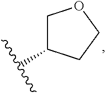 [SMILES: *[C@H]1CCOC1]

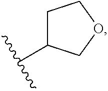 [SMILES: *C1CCOC1]

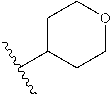 [SMILES: *C1CCOCC1]

or

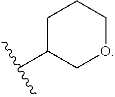 [SMILES: *C1CCCOC1]

[6]. The compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of any one of [1] to [5], wherein, R_{S1} at each occurrence is independently selected from deuterium, halogen, —C₁₋₆alkyl, haloC₁₋₆alkyl, —CN, —NH₂, —NH(C₁₋₃alkyl), —N(C₁₋₃alkyl)₂, —OH, —OC₁₋₃alkyl, —SH, —SC₁₋₃alkyl, —C(═O)C₁₋₃alkyl, —C(═O)OH, —C(═O)OC₁₋₃alkyl, —OC(═O)C₁₋₃alkyl, 3-6 membered cycloalkyl, 3-6 membered heterocyclyl or 5-6 membered heteroaryl; wherein said —C₁₋₆alkyl, haloC₁₋₆alkyl, 3-6 membered cycloalkyl, 3-6 membered heterocyclyl or 5-6 membered heteroaryl is optionally independently substituted with one or more substituents selected from deuterium, halogen, —C₁₋₆alkyl, haloC₁₋₆alkyl, —CN, —NH₂, —NH(C₁₋₃alkyl), —N(C₁₋₃alkyl)₂, —OH, —OC₁₋₃alkyl, —SH, —SC₁₋₃alkyl, —C(═O)C₁₋₃alkyl, —C(═O)OH, —C(═O)OC₁₋₃alkyl, —OC(═O)C₁₋₃alkyl, 3-6 membered cycloalkyl or 3-6 membered heterocyclyl;
Optionally, two R_{S1} together with the carbon atom to which they are both attached form

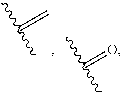

a 3-6 membered carbocyclic ring or a 3-6 heterocyclic ring; wherein, said

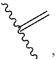

3-10 membered carbocylic ring or 3-10 heterocyclic ring is optionally substituted with one or more substitutents selected from deuterium, halogen, —C₁₋₆alkyl, haloC₁₋₆alkyl, —CN, —NH₂, —NH(C₁₋₃alkyl), —N(C₁₋₃alkyl)₂, —OH, —OC₁₋₃alkyl, —SH, —SC₁₋₃alkyl, —C(═O)C₁₋₃alkyl, —C(═O)OH, —C(═O)OC₁₋₃alkyl, —OC(═O)C₁₋₃alkyl, 3-6 membered cycloalkyl or 3-6 membered heterocyclyl;
Optionally, two adjacent R_{S1} together with the carbon atoms to which they are respectively attached form a 3-6 membered carbocyclic ring, or a 3-6 membered heterocyclic ring, wherein, each of rings is independently optionally substituted with one or more substitutents selected from deuterium, halogen, —C₁₋₆alkyl, haloC₁₋₆alkyl, —CN, —NH₂, —NH(C₁₋₃alkyl), —N(C₁₋₃alkyl)₂, —OH, —OC₁₋₃alkyl, —SH, —SC₁₋₃alkyl, —C(═O)C₁₋₃alkyl, —C(═O)OH, —C(═O)OC₁₋₃alkyl, —OC(═O)C₁₋₃alkyl, 3-6 membered cycloalkyl or 3-6 membered heterocyclyl.

[7]. The compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of any one of [1] to [6], wherein, R_{S1} at each occurrence is independently selected from deuterium, —F, —Cl, methyl, ethyl, propyl, isopropyl, n-butyl, isobutyl, sec-butyl, tert-butyl, n-pentyl, isopentyl, sec-pentyl, tert-pentyl, n-hexyl, iso-hexyl, sec-hexyl, tert-hexyl, halomethyl, haloethyl, —CN, —NH₂, —NHCH₃, —N(CH₃)₂, —OH, methoxy, ethoxy, —SH, —SCH₃, —C(═O)CH₃, —C(═O)OH, —C(═O)OCH₃ or —OC(═O)CH₃; wherein said methyl, ethyl, propyl, isopropyl, n-butyl, isobutyl, sec-butyl, tert-butyl, n-pentyl, isopentyl, sec-pentyl, tert-pentyl, n-hexyl, iso-hexyl, sec-hexyl, tert-hexyl, halomethyl, haloethyl, methoxy, ethoxy is optionally independently substituted with 1, 2, 3, 4, 5 or 6 substituents selected from deuterium, —F, methyl, ethyl, propyl, isopropyl, —CH₂F, —CHF₂, —CF₃, —CN, —NH₂, —NHCH₃, —N(CH₃)₂, —OH, —OCH₃, —SH, —SCH₃, —C(═O)CH₃, —C(═O)OH, —C(═O)OCH₃, —C(═O)OCH₂CH₃ or —OC(═O)CH₃;
Optionally two R_{S1} together with the carbon atom to which they are both attached form

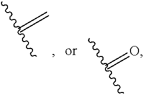

wherein, said

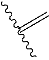

is optionally substituted with one or more substituents selected from deuterium, —F, —Cl, methyl, ethyl, propyl, isopropyl, —CH₂F, —CHF₂ or —CF₃.

[8]. The compound of Formula (IB), a stereoisomer thereof, a pharmaceutically acceptable salt thereof, a pharmaceutically acceptable salt of the stereoisomer thereof, a prodrug thereof, a deuterated molecule thereof or a conjugated form thereof of any one of [1] to [7], wherein, R_{S1} at each occurrence is independently selected from -D, —F, —CH₃, —CD₃, —CH₂F, —CHF₂, —CF₃, —CN, —CH₂CN, —OH, —OCH₃, —OCD₃, —NHCH₃, —SCH₃, —CH₂OCH₃, —C(═O)CH₃, —CH₂CH₃, —CHFCF₃,

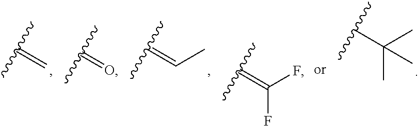

[9]. The compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of any one of [1] to [8], wherein, the moiety of

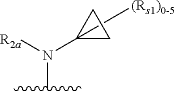

is selected from the moiety in the Table 2:

TABLE 2

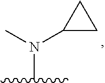 [SMILES: *N(C)C1CC1]

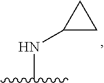 [SMILES: *NC1CC1]

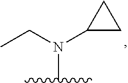 [SMILES: *N(CC)C1CC1]

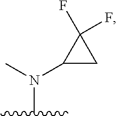 [SMILES: *N(C)C1CC1(F)F]

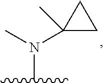 [SMILES: *N(C)C1(C)CC1]

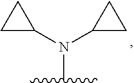 [SMILES: *N(C1CC1)C1CC1]

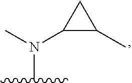

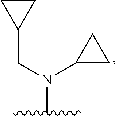 [SMILES: *N(CC1CC1)C1CC1]

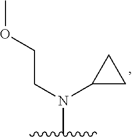 [SMILES: *N(CCOC)C1CC1]

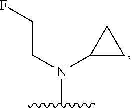 [SMILES: *N(CCF)C1CC1]

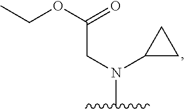 [SMILES: *N(CC(=O)OCC)C1CC1]

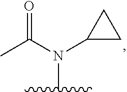 [SMILES: *N(C(C)=O)C1CC1]

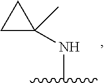 [SMILES: *NC1(C)CC1]

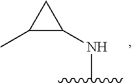 [SMILES: *NC1CC1C]

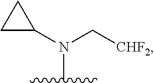 [SMILES: *N(CC(F)F)C1CC1]

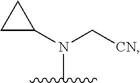 [SMILES: *N(CC#N)C1CC1]

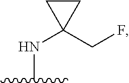 [SMILES: *NC1(CF)CC1]

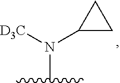 [SMILES: *N(C1CC1)C([2H])([2H])[2H]]

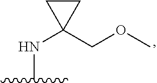 [SMILES: *NC1(COC)CC1]

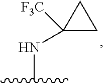 [SMILES: *NC1(C(F)(F)F)CC1]

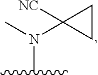 [SMILES: *N(C)C1(C#N)CC1]

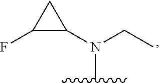 [SMILES: *N(CC)C1CC1F]

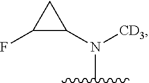 [SMILES: *N(C1CC1F)C([2H])([2H])[2H]]

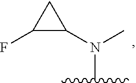 [SMILES: *N(C)C1CC1F]

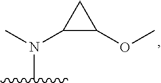

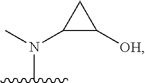 [SMILES: *N(C)C1CC1O]

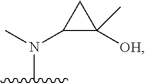 [SMILES: *N(C)C1CC1(C)O]

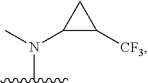 [SMILES: *N(C)C1CC1C(F)(F)F]

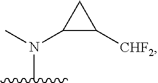 [SMILES: *N(C)C1CC1C(F)F]

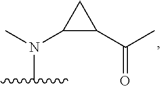 [SMILES: *N(C)C1CC1C(C)=O]

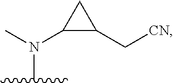 [SMILES: *N(C)C1CC1CC#N]

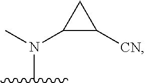 [SMILES: *N(C)C1CC1C#N]

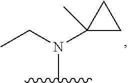 [SMILES: *N(CC)C1(C)CC1]

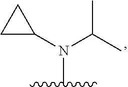 [SMILES: *N(C(C)C)C1CC1]

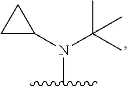 [SMILES: *N(C1CC1)C(C)(C)C]

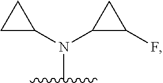 [SMILES: *N(C1CC1)C1CC1F]

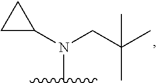 [SMILES: *N(CC(C)(C)C)C1CC1]

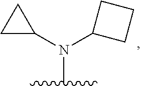 [SMILES: *N(C1CCC1)C1CC1]

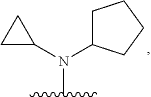 [SMILES: *N(C1CCCC1)C1CC1]

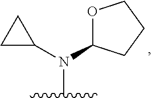 [SMILES: *N(C1CC1)[C@@H]1CCCO1]

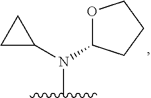 [SMILES: *N(C1CC1)[C@H]1CCCO1]

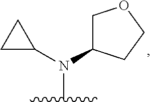 [SMILES: *N(C1CC1)[C@@H]1CCOC1]

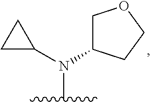 [SMILES: *N(C1CC1)[C@H]1CCOC1]

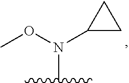 [SMILES: *N(OC)C1CC1]

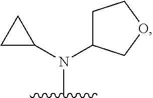 [SMILES: *N(C1CC1)C1CCOC1]

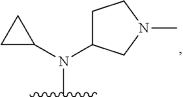 [SMILES: *N(C1CC1)C1CCN(C)C1]

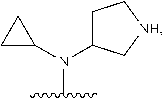 [SMILES: *N(C1CC1)C1CCNC1]

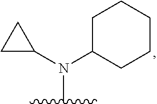 [SMILES: *N(C1CCCCC1)C1CC1]

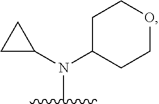 [SMILES: *N(C1CCOCC1)C1CC1]

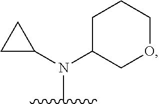 [SMILES: *N(C1CC1)C1CCCOC1]

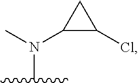 [SMILES: *N(C)C1CC1Cl]

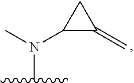

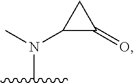 [SMILES: *N(C)C1CC1=O]

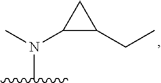 [SMILES: *N(C)C1CC1CC]

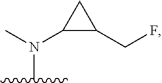 [SMILES: *N(C)C1CC1CF]

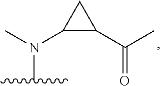 [SMILES: *N(C)C1CC1C(C)=O]

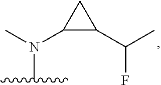 [SMILES: *N(C)C1CC1C(C)F]

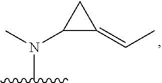 [SMILES: *N(C)C1C/C1=C\C]

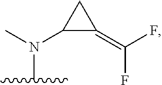 [SMILES: *N(C)C1CC1=C(F)F]

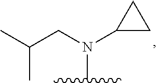 [SMILES: *N(CC(C)C)C1CC1]

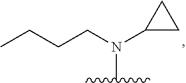 [SMILES: *N(CCCC)C1CC1]

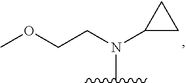 [SMILES: *N(CCOC)C1CC1]

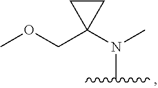

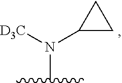 [SMILES: *N(C1CC1)C([2H])([2H])[2H]]

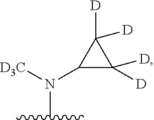

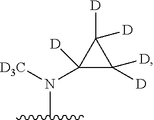

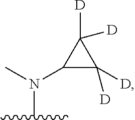

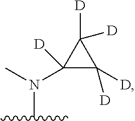

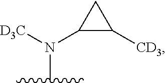 [SMILES: *N(C1CC1C([2H])([2H])[2H])C([2H])([2H])[2H]]

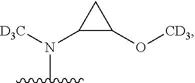 [SMILES: *N(C1CC1OC([2H])([2H])[2H])C([2H])([2H])[2H]]

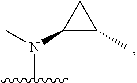

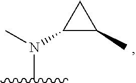

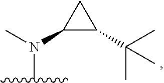 [SMILES: *N(C)[C@H]1C[C@@H]1C(C)(C)C]

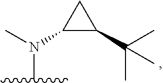 [SMILES: *N(C)[C@@H]1C[C@H]1C(C)(C)C]

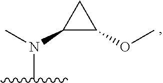

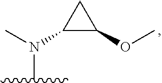

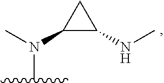

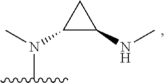

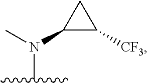 [SMILES: *N(C)[C@H]1C[C@@H]1C(F)(F)F]

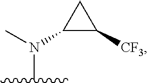 [SMILES: *N(C)[C@@H]1C[C@H]1C(F)(F)F]

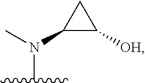 [SMILES: *N(C)[C@H]1C[C@@H]1O]

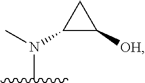 [SMILES: *N(C)[C@@H]1C[C@H]1O]

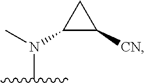 [SMILES: *N(C)[C@@H]1C[C@H]1C#N]

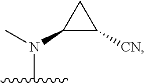 [SMILES: *N(C)[C@H]1C[C@@H]1C#N]

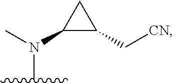 [SMILES: *N(C)[C@H]1C[C@@H]1CC#N]

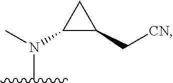 [SMILES: *N(C)[C@@H]1C[C@H]1CC#N]

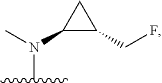 [SMILES: *N(C)[C@H]1C[C@@H]1CF]

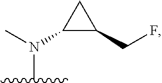 [SMILES: *N(C)[C@@H]1C[C@H]1CF]

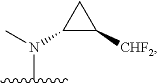 [SMILES: *N(C)[C@@H]1C[C@H]1C(F)F]

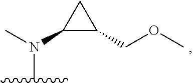

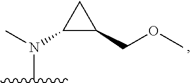

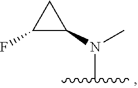 [SMILES: *N(C)[C@@H]1C[C@H]1F]

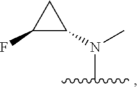 [SMILES: *N(C)[C@H]1C[C@@H]1F]

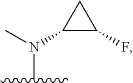 [SMILES: *N(C)[C@@H]1C[C@@H]1F]

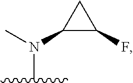 [SMILES: *N(C)[C@H]1C[C@H]1F]

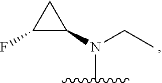 [SMILES: *N(CC)[C@@H]1C[C@H]1F]

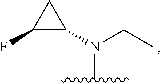 [SMILES: *N(CC)[C@H]1C[C@@H]1F]

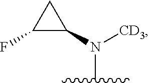 [SMILES: *N([C@@H]1C[C@H]1F)C([2H])([2H])[2H]]

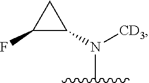 [SMILES: *N([C@H]1C[C@@H]1F)C([2H])([2H])[2H]]

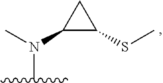

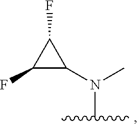 [SMILES: *N(C)C1[C@@H](F)[C@@H]1F]

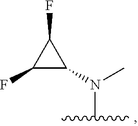 [SMILES: *N(C)[C@H]1[C@H](F)[C@@H]1F]

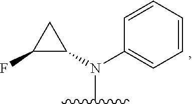 [SMILES: *N(c1ccccc1)[C@H]1C[C@@H]1F]

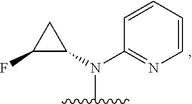 [SMILES: *N(c1ccccn1)[C@H]1C[C@@H]1F]

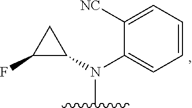 [SMILES: *N(c1ccccc1C#N)[C@H]1C[C@@H]1F]

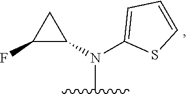 [SMILES: *N(c1cccs1)[C@H]1C[C@@H]1F]

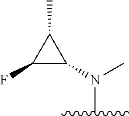 [SMILES: *N(C)[C@H]1[C@@H](C)[C@@H]1F]

or

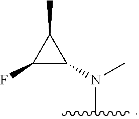 [SMILES: *N(C)[C@H]1[C@H](C)[C@@H]1F]

[10]. The compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of any one of [2] to [9], wherein, the moiety of

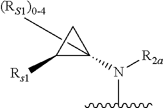

is selected from the moiety in the Table 3:

TABLE 3

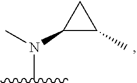

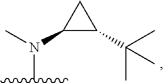 [SMILES: *N(C)[C@H]1C[C@@H]1C(C)(C)C]

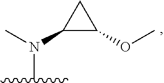

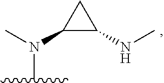

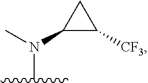 [SMILES: *N(C)[C@H]1C[C@@H]1C(F)(F)F]

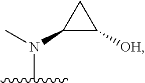 [SMILES: *N(C)[C@H]1C[C@@H]1O]

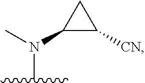 [SMILES: *N(C)[C@H]1C[C@@H]1C#N]

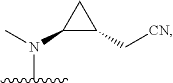 [SMILES: *N(C)[C@H]1C[C@@H]1CC#N]

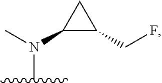 [SMILES: *N(C)[C@H]1C[C@@H]1CF]

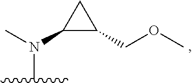

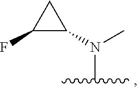 [SMILES: *N(C)[C@H]1C[C@@H]1F]

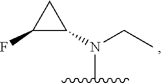 [SMILES: *N(CC)[C@H]1C[C@@H]1F]

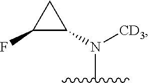 [SMILES: *N([C@H]1C[C@@H]1F)C([2H])([2H])[2H]]

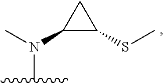

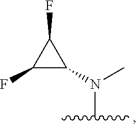 [SMILES: *N(C)[C@H]1[C@H](F)[C@@H]1F]

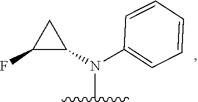 [SMILES: *N(c1ccccc1)[C@H]1C[C@@H]1F]

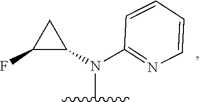 [SMILES: *N(c1ccccn1)[C@H]1C[C@@H]1F]

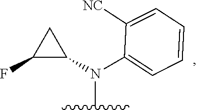 [SMILES: *N(c1ccccc1C#N)[C@H]1C[C@@H]1F]

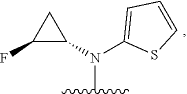 [SMILES: *N(c1cccs1)[C@H]1C[C@@H]1F]

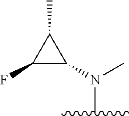 [SMILES: *N(C)[C@H]1[C@@H](C)[C@@H]1F]

or

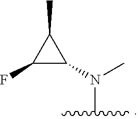 [SMILES: *N(C)[C@H]1[C@H](C)[C@@H]1F]

[11]. The compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of any one of [1] to [10], wherein, the compound is selected from any one of the following formulas:

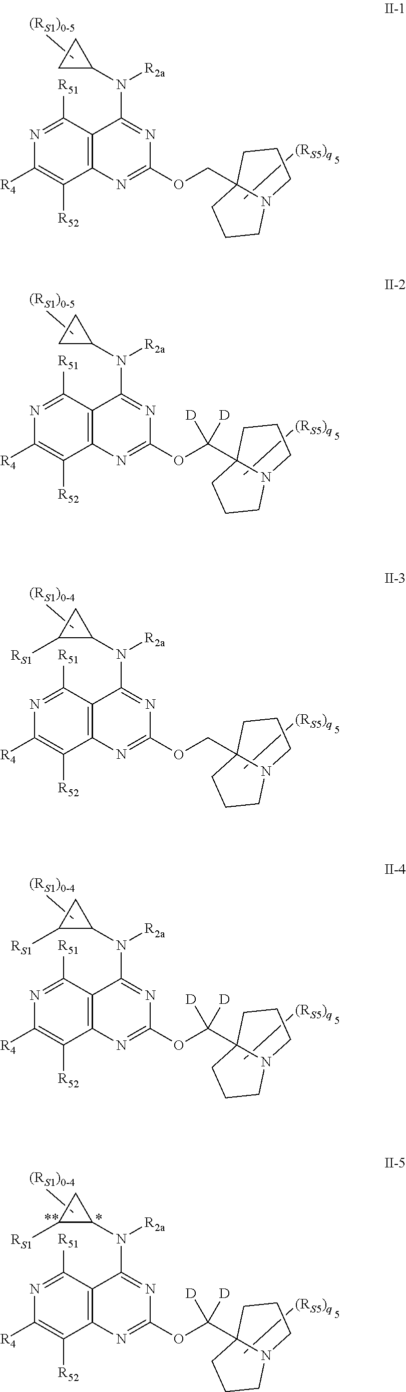

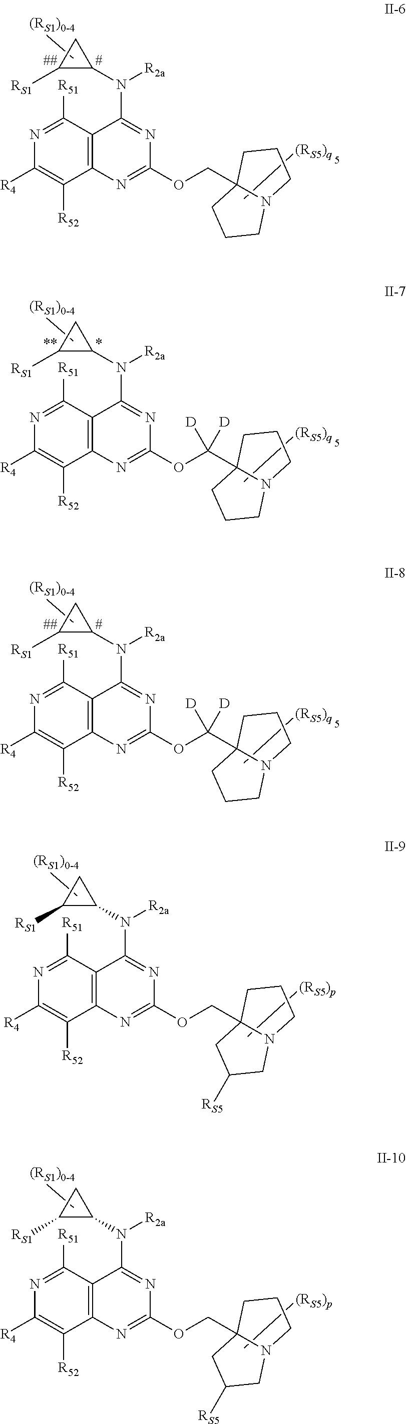

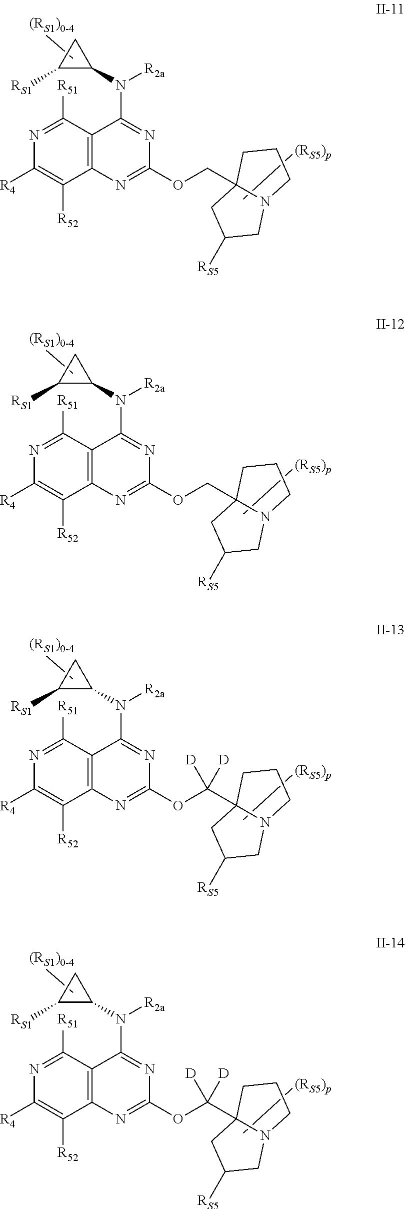

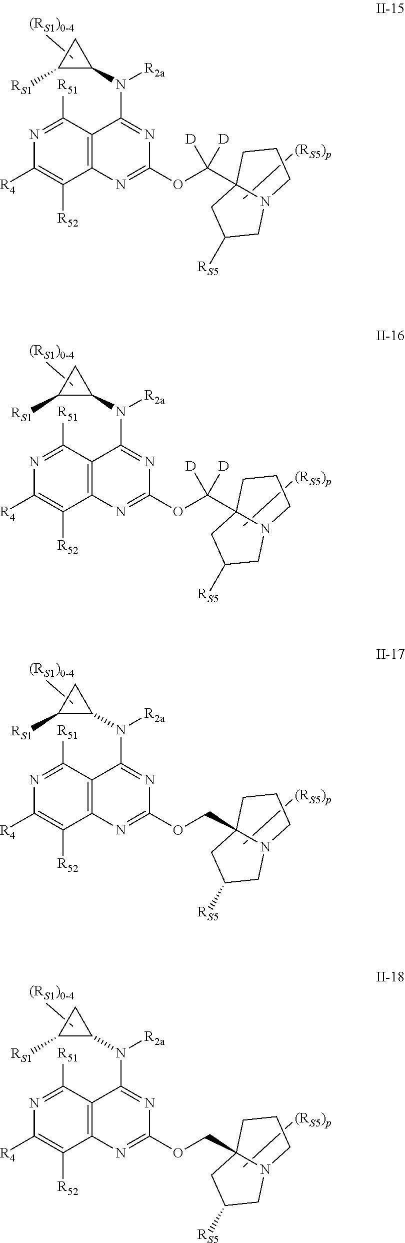

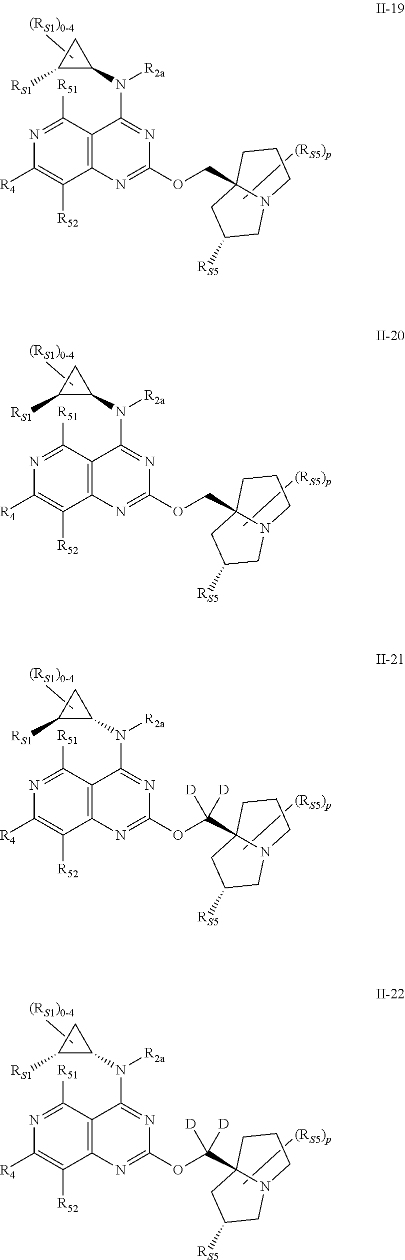

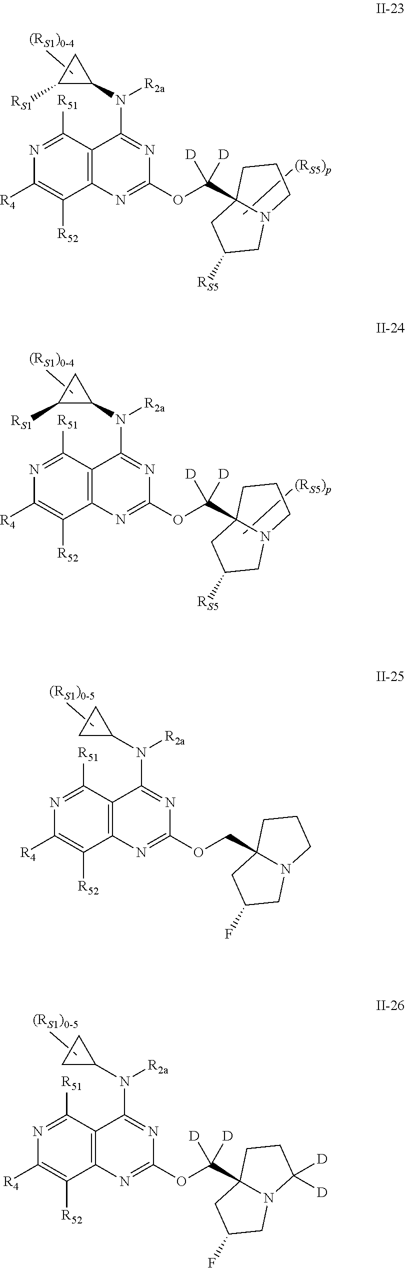

Wherein, R₂ₐ is selected from hydrogen, deuterium, —C₁₋₆alkyl, haloC₁₋₆alkyl, 3-6 membered cycloalkyl, 3-6 membered cycloalkenyl, phenyl, or 5-6 membered heteroaryl; wherein said —C₁₋₆alkyl, haloC₁₋₆alkyl, 3-6 membered cycloalkyl, 3-6 membered heterocyclyl, phenyl or 5-6 membered heteroaryl is optionally independently substituted with one or more substituents selected from deuterium, halogen, —C₁₋₃alkyl, haloC₁₋₃alkyl, —C₂₋₃alkenyl, —C₂₋₃alkynyl, —CN, —NH₂, —NH(C₁₋₃alkyl), —N(C₁₋₃alkyl)₂, —OH, —OC₁₋₃alkyl, —SH, —SC₁₋₃alkyl, —C(═O)C₁₋₃alkyl, —C(═O)OH, —C(═O)OC₁₋₃alkyl, —OC(═O)C₁₋₃alkyl, 3-6 membered cycloalkyl or 3-6 membered heterocyclyl;
R_{S1} at each occurrence is independently selected from deuterium, halogen, —C₁₋₆alkyl, haloC₁₋₆alkyl, —CN, —NH₂, —NH(C₁₋₃alkyl), —N(C₁₋₃alkyl)₂, —OH, —OC₁₋₃alkyl, —SH, —SC₁₋₃alkyl, —C(═O)C₁₋₃alkyl, —C(═O)OH, —C(═O)OC₁₋₃alkyl, —OC(═O)C₁₋₃alkyl, 3-6 membered cycloalkyl, 3-6 membered heterocyclyl or 5-6 membered heteroaryl; wherein said —C₁₋₆alkyl, haloC₁₋₆alkyl, 3-6 membered cycloalkyl, 3-6 membered heterocyclyl or 5-6 membered heteroaryl is optionally independently substituted with one or more substituents selected from deuterium, halogen, —C₁₋₆alkyl, haloC₁₋₆alkyl, —CN, —NH₂, —NH(C₁₋₃alkyl), —N(C₁₋₃alkyl)₂, —OH, —OC₁₋₃alkyl, —SH, —SC₁₋₃alkyl, —C(═O)C₁₋₃alkyl, —C(═O)OH, —C(═O)OC₁₋₃alkyl, —OC(═O)C₁₋₃alkyl, 3-6 membered cycloalkyl or 3-6 membered heterocyclyl;
Optionally, two R_{S1} together with the carbon atom to which they are both attached form

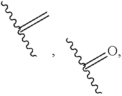

a 3-6 membered carbocyclic ring or a 3-6 heterocyclic ring; wherein, said

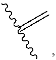

3-10 membered carbocylic ring or 3-10 heterocyclic ring is optionally substituted with one or more substitutents selected from deuterium, halogen, —C₁₋₆alkyl, haloC₁₋₆alkyl, —CN, —NH₂, —NH(C₁₋₃alkyl), —N(C₁₋₃alkyl)₂, —OH, —OC₁₋₃alkyl, —SH, —SC₁₋₃alkyl, —C(═O)C₁₋₃alkyl, —C(═O)OH, —C(═O)OC₁₋₃alkyl, —OC(═O)C₁₋₃alkyl, 3-6 membered cycloalkyl or 3-6 membered heterocyclyl;
Optionally, two adjacent R_{S1} together with the carbon atoms to which they are respectively attached form a 3-6 membered carbocyclic ring, or a 3-6 membered heterocyclic ring, wherein, each of rings is independently optionally substituted with one or more substitutents selected from deuterium, halogen, —C₁₋₆alkyl, haloC₁₋₆alkyl, —CN, —NH₂, —NH(C₁₋₃alkyl), —N(C₁₋₃alkyl)₂, —OH, —OC₁₋₃alkyl, —SH, —SC₁₋₃alkyl, —C(═O)C₁₋₃alkyl, —C(═O)OH, —C(═O)OC₁₋₃alkyl, —OC(═O)C₁₋₃alkyl, 3-6 membered cycloalkyl or 3-6 membered heterocyclyl.

R_{S1} linked with the carbon atom indicated with ** and NR₂ₐ linked with the carbon atom indicated with * are in a trans configuration;
R_{S1} linked with the carbon atom indicated with ## and NR₂ₐ linked with the carbon atom indicated with # are in a cis configuration;
p is selected from 0, 1, 2 or 3.

[12]. The compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of any one of [1] to [11], wherein, R_{S5} at each occurrence is independently selected from deuterium, halogen, —C₁₋₆alkyl, haloC₁₋₆alkyl, haloC₁₋₆alkoxy, —C₂₋₃alkenyl, —CN, —N(R₆₆)₂, —OR₆₆, —SR₆₆, —C(═O)R₆₇, —C(═O)OR₆₆, —OC(═O)R₆₇, —C(═O)N(R₆₆)₂, —NR₆₆C(═O)R₆₇, —OC(═O)OR₆₆, —NR₆₆C(═O)OR₆₆, —OC(═O)N(R₆₆)₂, —NR₆₆C(═O)N(R₆₆)₂, 3-8 membered cycloalkyl, 4-8 membered heterocyclyl containing 1, 2 or 3 heteroatoms selected from N, O or S or

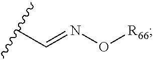

wherein, said —C₁₋₆alkyl is substituted with 1, 2 or 3 substituents selected from deuterium, halogen, —C₁₋₆alkyl, haloC₁₋₆alkyl, haloC₁₋₆alkoxy, —CN, oxo, —N(R₆₈)₂, —OR₆₈, —C(═O)R₆₈, —C(═O)OR₆₈, —OC(═O)R₆₈, —C(—)NR₆₈)₂, —NR₆₈C(═O)R₆₉, —OC(═O)OR₆₈, —NR₆₈C(═O)OR₆₉, —OC(═O)N(R₆₈)₂, —OC(═S)N(R₆₈)₂, —NR₆₈C(═O)N(R₆₈)₂, —NR₆₈S(═O)₂R₆₉, 3-6 membered cycloalkyl or 4-6 membered heterocyclyl; said 4-8 membered heterocyclyl is substituted with 1, 2 or 3 substituents selected from deuterium or —OR₆₈; said haloC₁₋₆alkyl is substituted with 1, 2 or 3 substituents selected from deuterium, —OR₆₈ or —C(═O)OR₆₈; said —C₂₋₃alkenyl is substituted with 1 substituents selected from deuterium or —C(═O)NR₆₈R₆₉;
Optionally, two R₈₅ together with the carbon atom to which they are both attached form

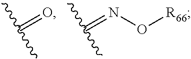

Each of (R₆₆ or R₆₇) is independently selected from hydrogen; deuterium; —C₁₋₆alkyl; haloC₁₋₆alkyl; or —C₁₋₆alkyl substituted with 1 or 2 substituents selected from —C(═O)N(C₁₋₆alkyl)₂, —OC₁₋₆alkyl, —C(═O)OC₁₋₆alkyl, —NHC₁₋₆alkyl or —N(C₁₋₆alkyl)₂;
Each of (R₆₈ or R₆₉) is independently selected from hydrogen; deuterium; —C₁₋₆alkyl; haloC₁₋₆alkyl; 5 membered heteroaryl; cyclopropyl; cyclopentyl; cyclohexyl; 5 membered heterocyclyl; 6 membered heterocyclyl; 5-membered heteroaryl; 6 membered heteroaryl; or —C₁₋₆alkyl substituted with 1, or 2 substituents selected from deuterium, —OC₁₋₆alkyl, —NHC₁₋₆alkyl, —N(C₁₋₆alkyl)₂ or —C(═O)N(C₁₋₆alkyl)₂; wherein said 5 membered heteroaryl, cyclopropyl, cyclopentyl, cyclohexyl, 5 membered heterocyclyl, 6 membered heterocyclyl, 5-membered heteroaryl or 6 membered heteroaryl is optionally substituted with 1 or 2 substitutents selected from deuterium, —C₁₋₃alkyl, —OH, —CN, —NH₂, —NH(C₁₋₃alkyl), —N(C₁₋₃alkyl)₂, —OC₁₋₃alkyl or cyclopropyl;
Optionally, two R₆₆ together with the nitrogen atom to which they are both attached form a 3-6 membered heterocyclic ring;
Optionally, two R₆₈ together with the nitrogen atom to which they are both attached form a 3-6 membered heterocyclic ring.

[13]. The compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of any one of [1] to [12], wherein, R_{S5} at each occurrence is independently selected from deuterium, —F, —C₁₋₃alkyl, haloC₁₋₃alkyl, haloC₁₋₃alkoxy, —C₂₋₆alkenyl, —C₂₋₆alkynyl, —CN, —N(R₆₆)₂, —OR₆₆, —SR₆₆, —C(═O)R₆₇, —C(═O)OR₆₆, —OC(═O)R₆₇, —C(═O)N(R₆₆)₂, —NR₆₆C(═O)R₆₇, —OC(═O)OR₆₆, —NR₆₆C(═O)OR₆₆, —OC(═O)N(R₆₆)₂ or —NR₆₆C(═O)N(R₆₆)₂; wherein, said —C₁₋₃alkyl is substituted with 1, 2 or 3 substituents selected from deuterium, halogen, —C₁₋₃alkyl, haloC₁₋₃alkyl, haloC₁₋₃alkoxy, —CN, oxo, —N(R₆₈)₂, —OR₆₈, —C(═O)R₆₈, —C(═O)OR₆₈, —OC(═O)R₆₈, —C(═O)N(R₆₈)₂, —NR₆₈C(═O)R₆₉, —OC(═O)OR₆₈, —NR₆₈C(═O)OR₆₉, —OC(═O)N(R₆₈)₂, —OC(═S)N(R₆₈)₂, —NR₆₈C(═O)N(R₆₈)₂ or —NR₆₈S(═O)₂R₆₉;
Optionally, two R_{S5} together with the carbon atom to which they are both attached form

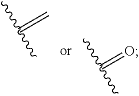

said

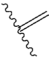

is optionally substituted with 1, 2, 3, 4, 5 or 6 substituents selected from deuterium, —F, —C₁₋₃alkyl or haloC₁₋₃alkyl;
R₆₆ or R₆₈ at each occurrence is independently selected from hydrogen, deuterium or —C₁₋₃alkyl;
Optionally, two R₆₆ together with the nitrogen atom to which they are both attached forms a 3-6 membered heterocyclic ring containing 1 or 2 heteroatoms selected from N, O, S, S(═O) or S(═O)₂;
Optionally, two R₆₈ together with the nitrogen atom to which they are both attached forms a 3-6 membered heterocyclic ring containing 1 or 2 heteroatoms selected from N, O, S, S(═O) or S(═O)₂;
R₆₇ or R₆₉ at each occurrence is independently selected from hydrogen, deuterium or —C₁₋₃alkyl.

[14]. The compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of any one of [1] to [13], wherein, R_{S5} at each occurrence is independently selected from deuterium, —F, —Cl, —CH₃, —CH₂CH₃, —CH₂CH₂CH₃, —CH(CH₃)₂, —CH═CH₂, —C≡CH, —C≡CCH₃, —C≡CD, —CH₂C≡CH, —CH₂F, —CHF₂, —CF₃, —CH₂CF₃, —CH₂CHF₂, —CH₂CH₂F, —CH₂CH₂CH₂F, —OH, —CH₂OH, —CH₂CH₂OH, —OCH₃, —OC(CH₃)₂, —OCH₂CH₃, —OCH(CH₃)₂, —OCF₃, —SH, —SCH₃, —SCF₃, —C(═O)CF₃, —CN, —NH₂, —N(CH₃)₂, —NHCH₂CH₃, —CH₂N(CH₃)₂, —NHC(═O)CH₃, —NHC(═O)OCH₃, —CH₂NHC(═O)OCH₃, —OC(═O)NHCH₃, —OC(═O)N(CH₃)₂, —CH₂OC(═O)N(CH₃)₂, —CH₂OC(═O)NHCH₃, —NHC(═O)N(CH₃)₂, —CH₂NHC(═O)N(CH₃)₂, —CH₂NHC(═O)CH₃, —CH₂OCH₃, or

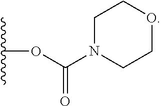 [SMILES: *OC(=O)N1CCOCC1]

[15]. The compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of any one of [1] to [14], wherein, q₅ is selected from 0, 1 or

[16]. The compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of any one of [1] to [15], wherein, q₅ is selected from 0 or 1.

[17]. The compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of am one of [1] to [16], wherein, the moiety of

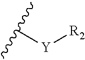

is selected from any moiety in the Table 4:

TABLE 4

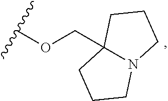 [SMILES: *OCC12CCCN1CCC2]

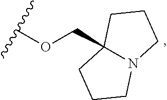

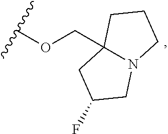 [SMILES: *OCC12CCCN1C[C@H](F)C2]

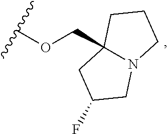 [SMILES: *OC[C@@]12CCCN1C[C@H](F)C2]

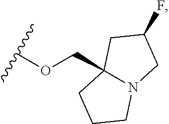 [SMILES: *OC[C@]12CCCN1C[C@H](F)C2]

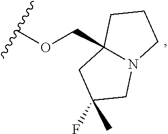 [SMILES: *OC[C@@]12CCCN1C[C@](C)(F)C2]

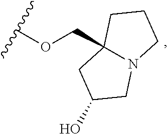 [SMILES: *OC[C@@]12CCCN1C[C@H](O)C2]

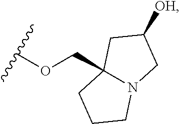 [SMILES: *OC[C@]12CCCN1C[C@H](O)C2]

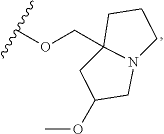 [SMILES: *OCC12CCCN1CC(OC)C2]

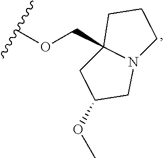 [SMILES: *OC[C@@]12CCCN1C[C@H](OC)C2]

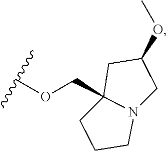 [SMILES: *OC[C@]12CCCN1C[C@H](OC)C2]

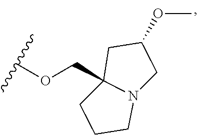 [SMILES: *OC[C@]12CCCN1C[C@@H](OC)C2]

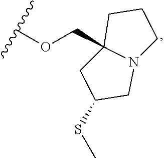 [SMILES: *OC[C@@]12CCCN1C[C@H](SC)C2]

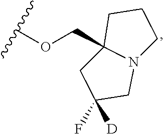 [SMILES: *OC[C@@]12CCCN1C[C@]([2H])(F)C2]

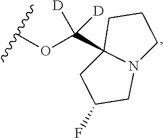 [SMILES: *OC([2H])([2H])[C@@]12CCCN1C[C@H](F)C2]

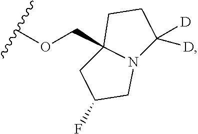

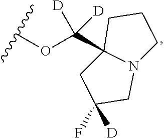 [SMILES: *OC([2H])([2H])[C@@]12CCCN1C[C@]([2H])(F)C2]

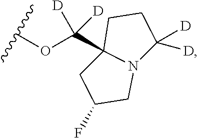

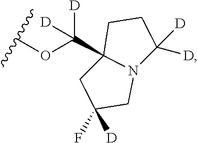

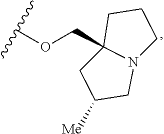 [SMILES: *OC[C@@]12CCCN1C[C@H](C)C2]

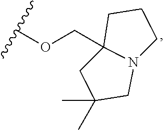 [SMILES: *OCC12CCCN1CC(C)(C)C2]

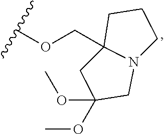 [SMILES: *OCC12CCCN1CC(OC)(OC)C2]

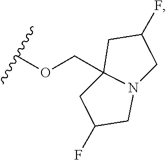 [SMILES: *OCC12CC(F)CN1CC(F)C2]

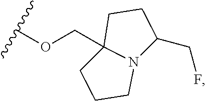 [SMILES: *OCC12CCCN1C(CF)CC2]

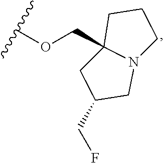 [SMILES: *OC[C@@]12CCCN1C[C@H](CF)C2]

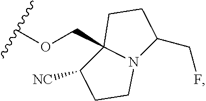 [SMILES: *OC[C@@]12CCC(CF)N1CC[C@@H]2C#N]

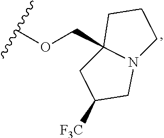 [SMILES: *OC[C@@]12CCCN1C[C@@H](C(F)(F)F)C2]

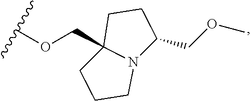 [SMILES: *OC[C@]12CCCN1[C@@H](COC)CC2]

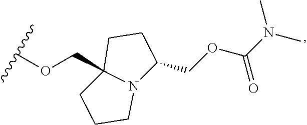 [SMILES: *OC[C@]12CCCN1[C@@H](COC(=O)N(C)C)CC2]

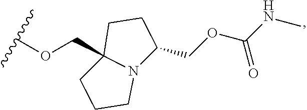 [SMILES: *OC[C@]12CCCN1[C@@H](COC(=O)NC)CC2]

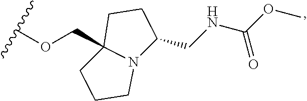 [SMILES: *OC[C@]12CCCN1[C@@H](CNC(=O)OC)CC2]

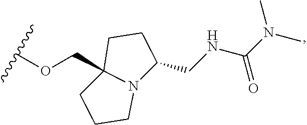 [SMILES: *OC[C@]12CCCN1[C@@H](CNC(=O)N(C)C)CC2]

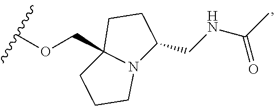 [SMILES: *OC[C@]12CCCN1[C@@H](CNC(C)=O)CC2]

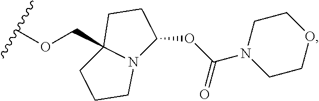 [SMILES: *OC[C@]12CCCN1[C@@H](OC(=O)N1CCOCC1)CC2]

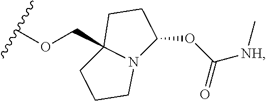 [SMILES: *OC[C@]12CCCN1[C@@H](OC(=O)NC)CC2]

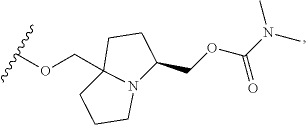 [SMILES: *OCC12CCCN1[C@H](COC(=O)N(C)C)CC2]

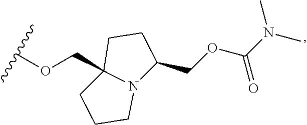 [SMILES: *OC[C@]12CCCN1[C@H](COC(=O)N(C)C)CC2]

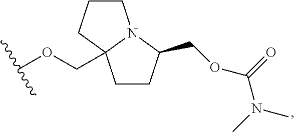 [SMILES: *OCC12CCCN1[C@@H](COC(=O)N(C)C)CC2]

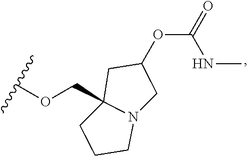 [SMILES: *OC[C@]12CCCN1CC(OC(=O)NC)C2]

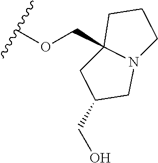 [SMILES: *OC[C@@]12CCCN1C[C@H](CO)C2]

or

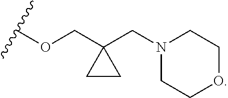 [SMILES: *OCC1(CN2CCOCC2)CC1]

[18]. The compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of any one of [11] to [17], wherein, the moiety of

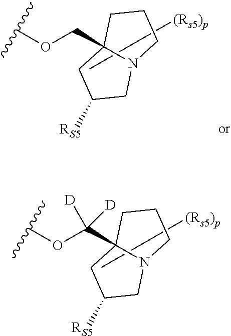

is selected from any moiety in the Table 5:

TABLE 5

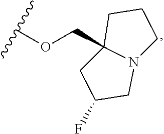 [SMILES: *OC[C@@]12CCCN1C[C@H](F)C2]

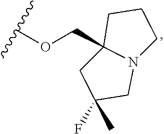 [SMILES: *OC[C@@]12CCCN1C[C@](C)(F)C2]

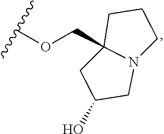 [SMILES: *OC[C@@]12CCCN1C[C@H](O)C2]

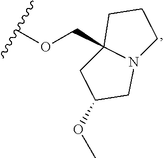 [SMILES: *OC[C@@]12CCCN1C[C@H](OC)C2]

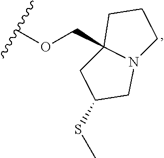 [SMILES: *OC[C@@]12CCCN1C[C@H](SC)C2]

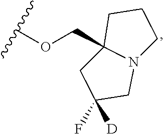 [SMILES: *OC[C@@]12CCCN1C[C@]([2H])(F)C2]

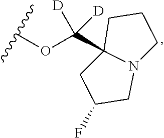 [SMILES: *OC([2H])([2H])[C@@]12CCCN1C[C@H](F)C2]

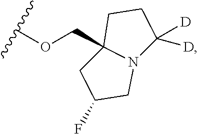

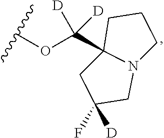 [SMILES: *OC([2H])([2H])[C@@]12CCCN1C[C@]([2H])(F)C2]

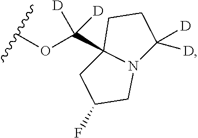

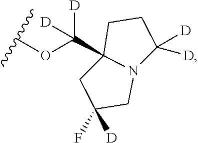

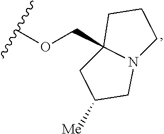 [SMILES: *OC[C@@]12CCCN1C[C@H](C)C2]

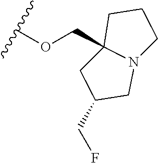 [SMILES: *OC[C@@]12CCCN1C[C@H](CF)C2]

or

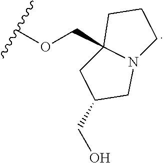 [SMILES: *OC[C@@]12CCCN1C[C@H](CO)C2]

[19]. The compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of any one of [1] to [8] wherein, R₄ is selected from any moiety in the Table 6:

TABLE 6

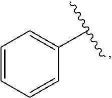 [SMILES: *c1ccccc1]

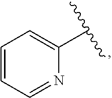 [SMILES: *c1ccccn1]

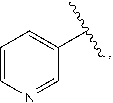 [SMILES: *c1cccnc1]

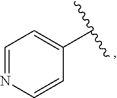 [SMILES: *c1ccncc1]

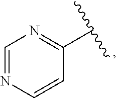 [SMILES: *c1ccncn1]

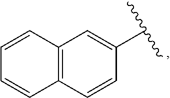 [SMILES: *c1ccc2ccccc2c1]

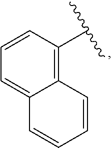 [SMILES: *c1cccc2ccccc12]

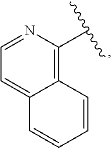 [SMILES: *c1nccc2ccccc12]

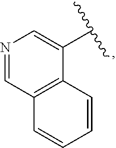 [SMILES: *c1cncc2ccccc12]

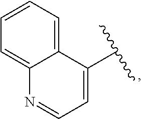 [SMILES: *c1ccnc2ccccc12]

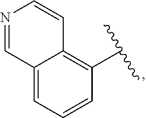 [SMILES: *c1cccc2cnccc12]

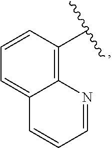 [SMILES: *c1cccc2cccnc12]

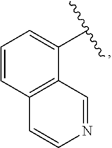 [SMILES: *c1cccc2ccncc12]

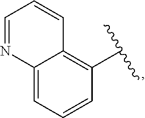 [SMILES: *c1cccc2ncccc12]

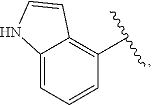 [SMILES: *c1cccc2[nH]ccc12]

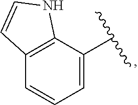 [SMILES: *c1cccc2cc[nH]c12]

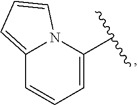 [SMILES: *c1cccc2cccn12]

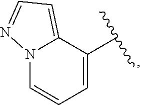 [SMILES: *c1cccn2nccc12]

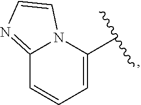 [SMILES: *c1cccc2nccn12]

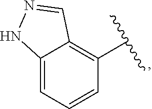 [SMILES: *c1cccc2[nH]ncc12]

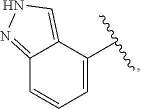 [SMILES: *c1cccc2n[nH]cc12]

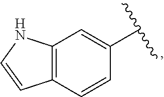 [SMILES: *c1ccc2cc[nH]c2c1]

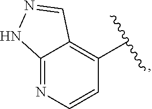 [SMILES: *c1ccnc2[nH]ncc12]

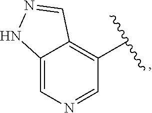 [SMILES: *c1cncc2[nH]ncc12]

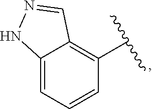 [SMILES: *c1cccc2[nH]ncc12]

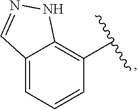 [SMILES: *c1cccc2cn[nH]c12]

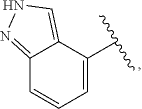 [SMILES: *c1cccc2n[nH]cc12]

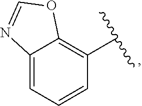 [SMILES: *c1cccc2ncoc12]

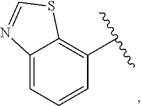 [SMILES: *c1cccc2ncsc12]

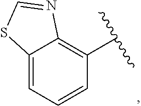 [SMILES: *c1cccc2scnc12]

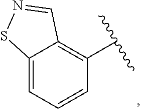 [SMILES: *c1cccc2sncc12]

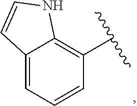 [SMILES: *c1cccc2cc[nH]c12]

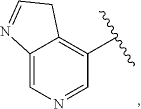 [SMILES: *c1cncc2c1CC=N2]

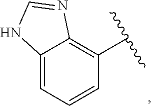 [SMILES: *c1cccc2[nH]cnc12]

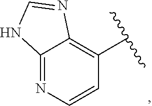 [SMILES: *c1ccnc2[nH]cnc12]

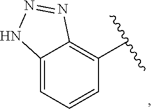 [SMILES: *c1cccc2[nH]nnc12]

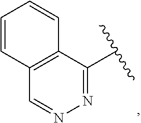 [SMILES: *c1nncc2ccccc12]

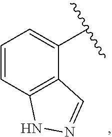 [SMILES: *c1cccc2[nH]ncc12]

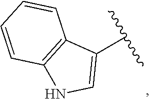 [SMILES: *c1c[nH]c2ccccc12]

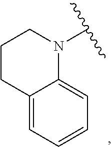 [SMILES: *N1CCCc2ccccc21]

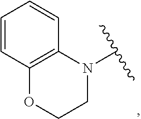 [SMILES: *N1CCOc2ccccc21]

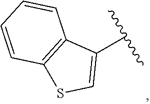 [SMILES: *c1csc2ccccc12]

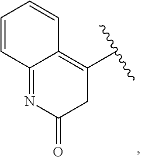 [SMILES: *C1=c2ccccc2=NC(=O)C1]

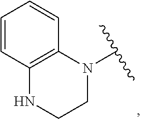 [SMILES: *N1CCNc2ccccc21]

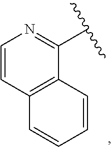 [SMILES: *c1nccc2ccccc12]

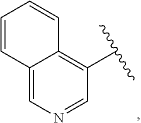 [SMILES: *c1cncc2ccccc12]

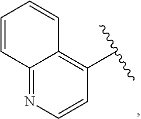 [SMILES: *c1ccnc2ccccc12]

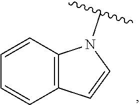 [SMILES: *n1ccc2ccccc21]

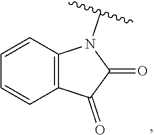 [SMILES: *N1C(=O)C(=O)c2ccccc21]

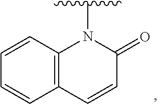 [SMILES: *n1c(=O)ccc2ccccc21]

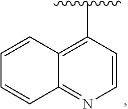 [SMILES: *c1ccnc2ccccc12]

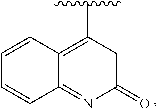 [SMILES: *C1=c2ccccc2=NC(=O)C1]

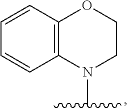 [SMILES: *N1CCOc2ccccc21]

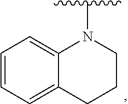 [SMILES: *N1CCCc2ccccc21]

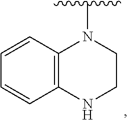 [SMILES: *N1CCNc2ccccc21]

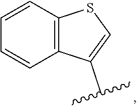 [SMILES: *c1csc2ccccc12]

or

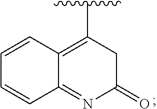

Wherein, each moiety in the Table 6 is independently optionally substituted with 1, 2, 3, 4, 5 or 6 R₄₁.

[20]. The compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of any one of [1] to [19], wherein, the compound is selected from any one of the following formulas:

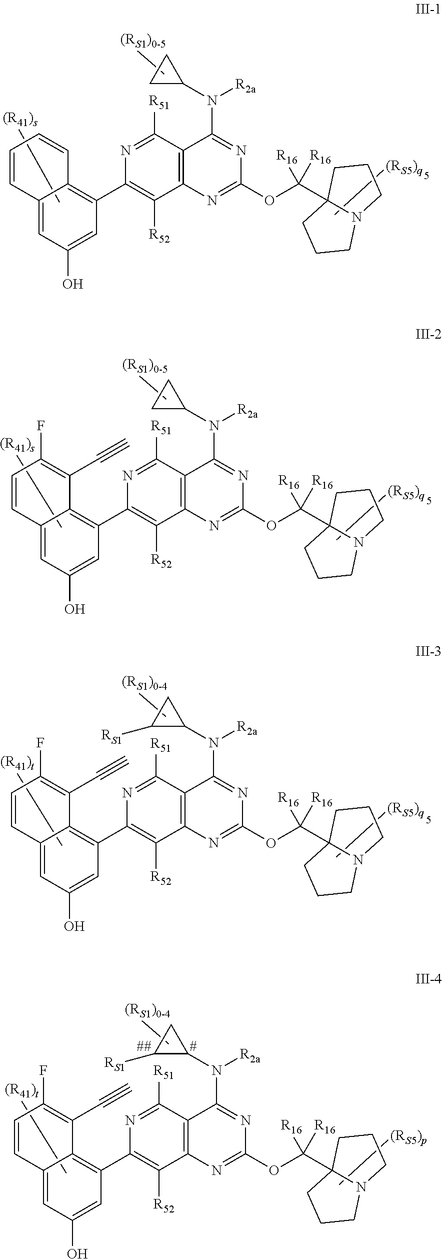

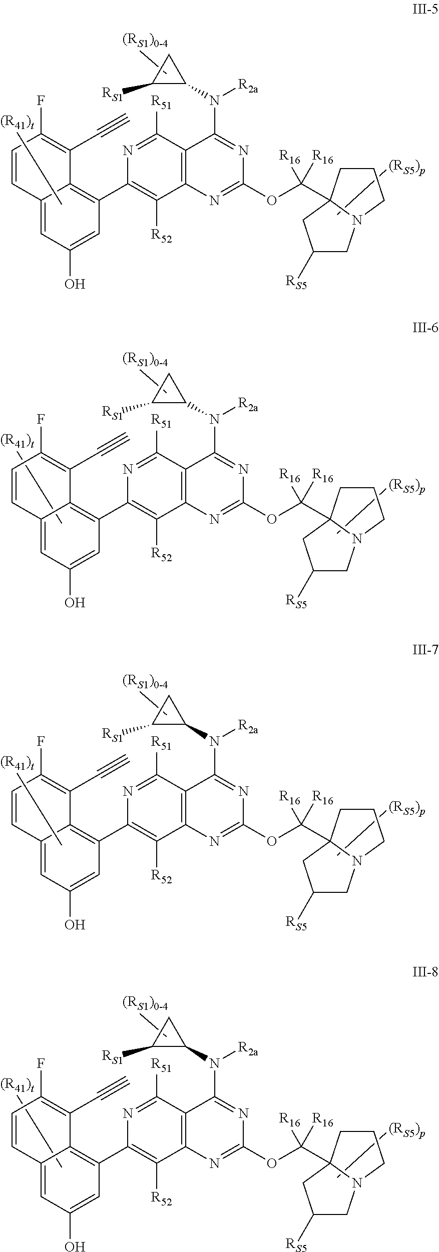

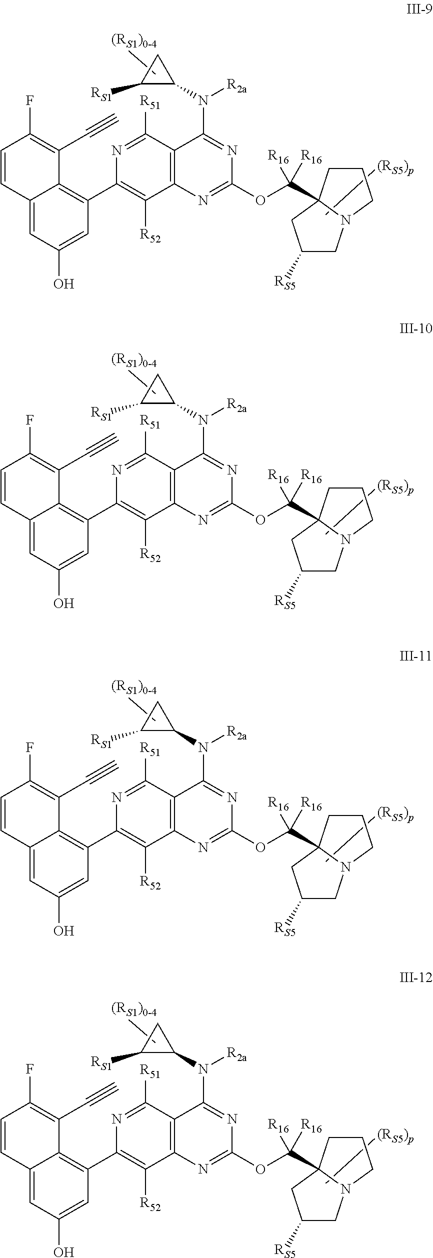

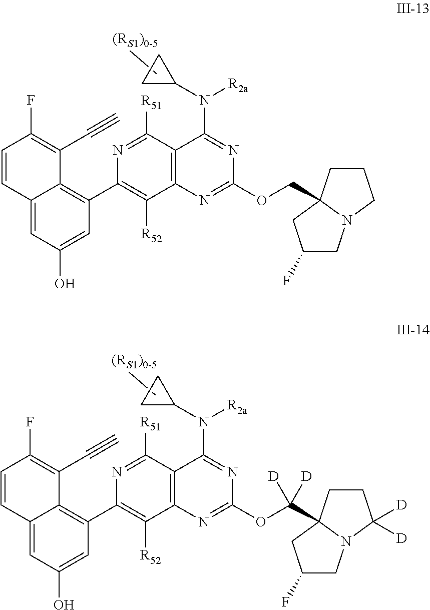

R₁₆ is selected from hydrogen or deuterium;
s is selected from 0, 1, 2, 3, 4, 5 or 6;
t is selected from 0, 1, 2, 3 or 4;
R_{S1} linked with the carbon atom indicated with ** and NR₂ₐ linked with the carbon atom indicated with * are in a trans configuration;
R_{S1} linked with the carbon atom indicated with ## and NR₂ₐ linked with the carbon atom indicated with # are in a cis configuration.

[21]. The compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of any one of [1] to [20], wherein, R₄₁ is independently selected from —F, —Cl, —C₁₋₃alkyl, haloC₁₋₃alkyl, haloC₁₋₃alkoxy, —C₂₋₃alkenyl, —C₂₋₃alkynyl, —CN, —NH₂, —NH(C₁₋₃alkyl), —N(C₁₋₃alkyl)₂, —OH, —O(C₁₋₃alkyl), —SH, —S(C₁₋₃alkyl), —S(═O)H, —S(═O)(C₁₋₃alkyl), 3-6 membered cycloalkyl or 3-6 membered heterocyclyl, wherein said —C₁₋₃alkyl, haloC₁₋₃alkyl, haloC₁₋₃alkoxy, —C₂₋₃alkenyl, —C₂₋₆alkynyl, —NH₂, —SH, 3-6 membered cycloalkyl or 3-6 membered heterocyclyl is independently optionally substituted with 1, 2 or 3 R₄₂;
Each of R₄₂ is independently selected from —F; —C₁₋₃alkyl; haloC₁₋₃alkyl; —CN; —OH; —NH₂; —NH(C₁₋₃alkyl); —N(C₁₋₃alkyl)₂; —OC₁₋₃alkyl; 3-6 membered cycloalkyl; or —C₁₋₃alkyl substituted with 1, 2 or 3 substituents selected from —F, haloC₁₋₃alkyl, —CN, —OH, —NH₂, —NH(C₁₋₃alkyl), —N(C₁₋₃alkyl)₂ or —OC₁₋₃alkyl.

[22]. The compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of any one of [1] to [21], wherein, R₄ is selected from any moiety in the Table 7:

TABLE 7

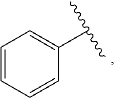 [SMILES: *c1ccccc1]

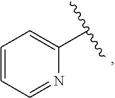 [SMILES: *c1ccccn1]

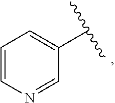 [SMILES: *c1cccnc1]

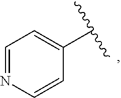 [SMILES: *c1ccncc1]

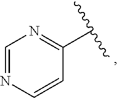 [SMILES: *c1ccncn1]

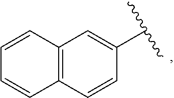 [SMILES: *c1ccc2ccccc2c1]

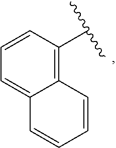 [SMILES: *c1cccc2ccccc12]

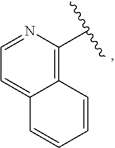 [SMILES: *c1nccc2ccccc12]

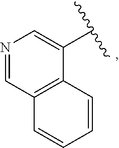 [SMILES: *c1cncc2ccccc12]

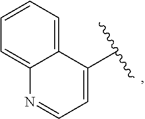 [SMILES: *c1ccnc2ccccc12]

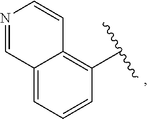 [SMILES: *c1cccc2cnccc12]

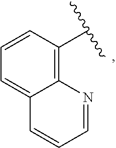 [SMILES: *c1cccc2cccnc12]

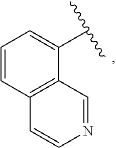 [SMILES: *c1cccc2ccncc12]

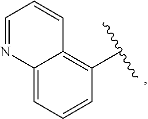 [SMILES: *c1cccc2ncccc12]

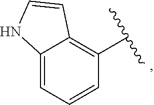 [SMILES: *c1cccc2[nH]ccc12]

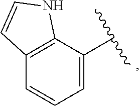 [SMILES: *c1cccc2cc[nH]c12]

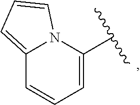 [SMILES: *c1cccc2cccn12]

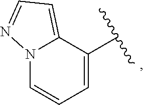 [SMILES: *c1cccn2nccc12]

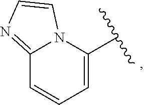 [SMILES: *c1cccc2nccn12]

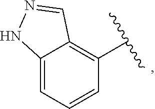 [SMILES: *c1cccc2[nH]ncc12]

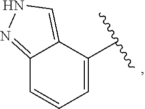 [SMILES: *c1cccc2n[nH]cc12]

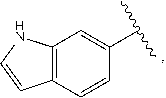 [SMILES: *c1ccc2cc[nH]c2c1]

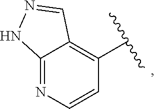 [SMILES: *c1ccnc2[nH]ncc12]

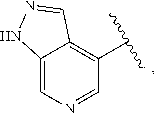 [SMILES: *c1cncc2[nH]ncc12]

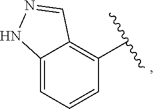 [SMILES: *c1cccc2[nH]ncc12]

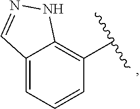 [SMILES: *c1cccc2cn[nH]c12]

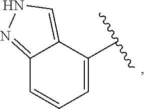 [SMILES: *c1cccc2n[nH]cc12]

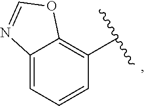 [SMILES: *c1cccc2ncoc12]

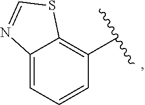 [SMILES: *c1cccc2ncsc12]

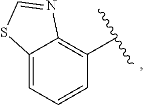 [SMILES: *c1cccc2scnc12]

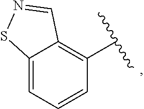 [SMILES: *c1cccc2sncc12]

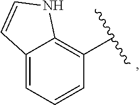 [SMILES: *c1cccc2cc[nH]c12]

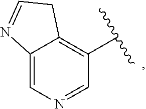 [SMILES: *c1cncc2c1CC=N2]

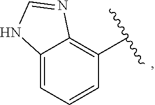 [SMILES: *c1cccc2[nH]cnc12]

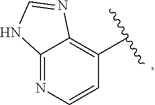 [SMILES: *c1ccnc2[nH]cnc12]

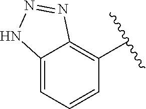 [SMILES: *c1cccc2[nH]nnc12]

or

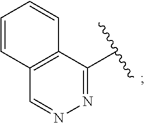

Wherein, said R₄ is independently optionally substituted with 1, 2, 3 or 4 R₄₁;
Each of R₄₁ is independently selected from any moiety in the Table 8:

TABLE 8
—F,
—Cl,
methyl,
ethyl,
isopropyl,
—CH═CH₂,
—C≡CH,
—C≡CCH₃,
—C≡CD,
—CH₂C≡CH,
—CHF₂,
—CF₃,
—CH₂CF₃,
—CH₂CHF₂,
—CH₂CH₂F,
—CH₂CH₂CH₂F,
—OCF₃,
—CN,
—CH₂CH₂CN,
—NH₂,
—N(CH₃)₂,
—NHCH₂CH₃,
—CH₂—N(CH₃)₂,
—OH,
—CH₂OH,
—CH₂CH₂OH,
—OCH₃,
—OC(CH₃)₂,
—CH₂CH(CH₃)₂,
—CH(CH₃)CH₂CH₃,
—CH₂OCH₃,
—SH,
—SCH₃,
—SCF₃,
—OCHF₂,
—CH(CF₃)OCH₃,
—C(CH₃)₂OH,
—CF(CH₃)₂,
—OCH(CH₃)₂,
cyclopropyl,

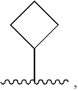 [SMILES: *C1CCC1]

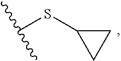 [SMILES: *SC1CC1]

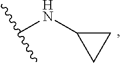 [SMILES: *NC1CC1]

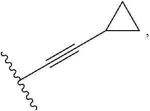 [SMILES: *C#CC1CC1]

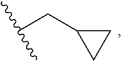 [SMILES: *CC1CC1]

or

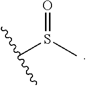 [SMILES: *S(C)=O]

[23]. The compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of any one of [1] to [22], wherein, R₄ is selected from any moiety in the Table 9:

TABLE 9

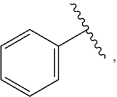 [SMILES: *c1ccccc1]

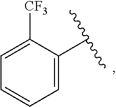 [SMILES: *c1ccccc1C(F)(F)F]

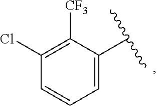 [SMILES: *c1cccc(Cl)c1C(F)(F)F]

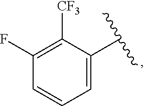 [SMILES: *c1cccc(F)c1C(F)(F)F]

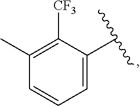 [SMILES: *c1cccc(C)c1C(F)(F)F]

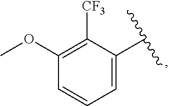 [SMILES: *c1cccc(OC)c1C(F)(F)F]

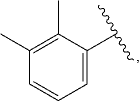 [SMILES: *c1cccc(C)c1C]

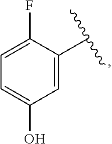 [SMILES: *c1cc(O)ccc1F]

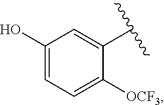 [SMILES: *c1cc(O)ccc1OC(F)(F)F]

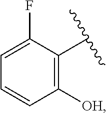 [SMILES: *c1c(O)cccc1F]

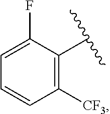 [SMILES: *c1c(F)cccc1C(F)(F)F]

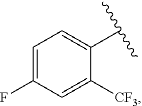 [SMILES: *c1ccc(F)cc1C(F)(F)F]

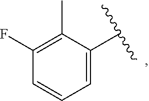 [SMILES: *c1cccc(F)c1C]

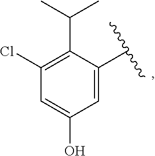 [SMILES: *c1cc(O)cc(Cl)c1C(C)C]

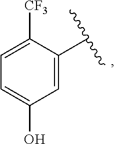 [SMILES: *c1cc(O)ccc1C(F)(F)F]

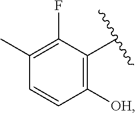 [SMILES: *c1c(O)ccc(C)c1F]

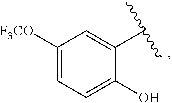 [SMILES: *c1cc(OC(F)(F)F)ccc1O]

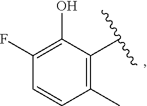 [SMILES: *c1c(C)ccc(F)c1O]

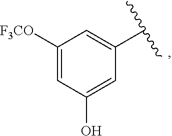 [SMILES: *c1cc(O)cc(OC(F)(F)F)c1]

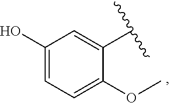 [SMILES: *c1cc(O)ccc1OC]

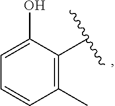 [SMILES: *c1c(C)cccc1O]

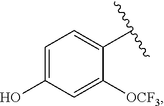 [SMILES: *c1ccc(O)cc1OC(F)(F)F]

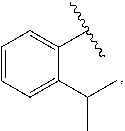 [SMILES: *c1ccccc1C(C)C]

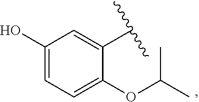 [SMILES: *c1cc(O)ccc1OC(C)C]

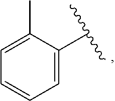 [SMILES: *c1ccccc1C]

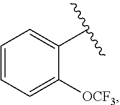 [SMILES: *c1ccccc1OC(F)(F)F]

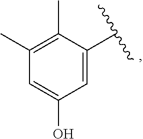 [SMILES: *c1cc(O)cc(C)c1C]

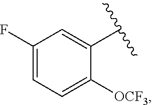 [SMILES: *c1cc(F)ccc1OC(F)(F)F]

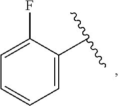 [SMILES: *c1ccccc1F]

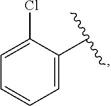 [SMILES: *c1ccccc1Cl]

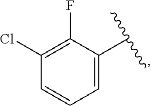 [SMILES: *c1cccc(Cl)c1F]

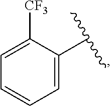 [SMILES: *c1ccccc1C(F)(F)F]

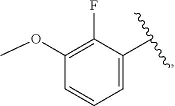 [SMILES: *c1cccc(OC)c1F]

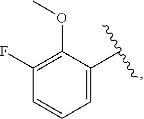 [SMILES: *c1cccc(F)c1OC]

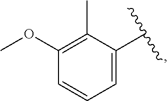 [SMILES: *c1cccc(OC)c1C]

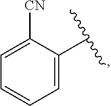 [SMILES: *c1ccccc1C#N]

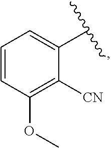 [SMILES: *c1cccc(OC)c1C#N]

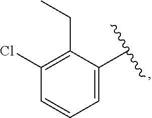 [SMILES: *c1cccc(Cl)c1CC]

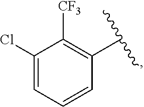 [SMILES: *c1cccc(Cl)c1C(F)(F)F]

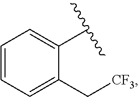 [SMILES: *c1ccccc1CC(F)(F)F]

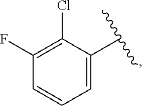 [SMILES: *c1cccc(F)c1Cl]

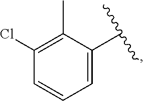 [SMILES: *c1cccc(Cl)c1C]

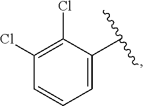 [SMILES: *c1cccc(Cl)c1Cl]

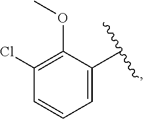 [SMILES: *c1cccc(Cl)c1OC]

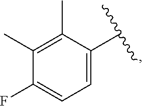 [SMILES: *c1ccc(F)c(C)c1C]

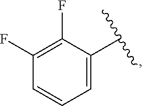 [SMILES: *c1cccc(F)c1F]

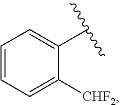 [SMILES: *c1ccccc1C(F)F]

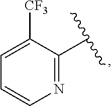 [SMILES: *c1ncccc1C(F)(F)F]

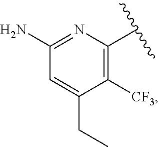 [SMILES: *c1nc(N)cc(CC)c1C(F)(F)F]

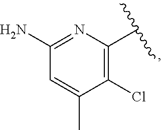 [SMILES: *c1nc(N)cc(C)c1Cl]

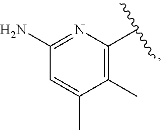 [SMILES: *c1nc(N)cc(C)c1C]

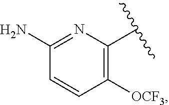 [SMILES: *c1nc(N)ccc1OC(F)(F)F]

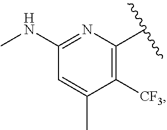 [SMILES: *c1nc(NC)cc(C)c1C(F)(F)F]

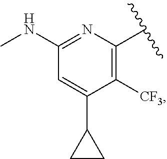 [SMILES: *c1nc(NC)cc(C2CC2)c1C(F)(F)F]

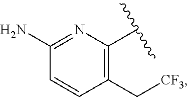 [SMILES: *c1nc(N)ccc1CC(F)(F)F]

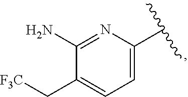 [SMILES: *c1ccc(CC(F)(F)F)c(N)n1]

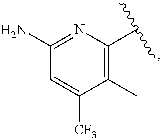 [SMILES: *c1nc(N)cc(C(F)(F)F)c1C]

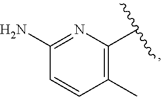 [SMILES: *c1nc(N)ccc1C]

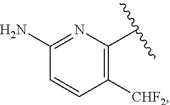 [SMILES: *c1nc(N)ccc1C(F)F]

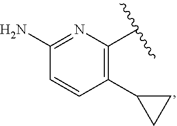 [SMILES: *c1nc(N)ccc1C1CC1]

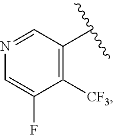 [SMILES: *c1cncc(F)c1C(F)(F)F]

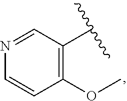 [SMILES: *c1cnccc1OC]

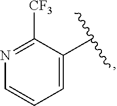 [SMILES: *c1cccnc1C(F)(F)F]

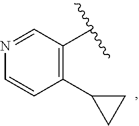 [SMILES: *c1cnccc1C1CC1]

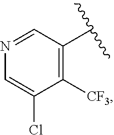 [SMILES: *c1cncc(Cl)c1C(F)(F)F]

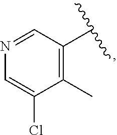 [SMILES: *c1cncc(Cl)c1C]

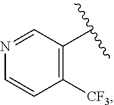 [SMILES: *c1cnccc1C(F)(F)F]

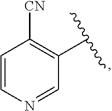 [SMILES: *c1cnccc1C#N]

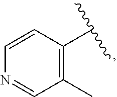 [SMILES: *c1ccncc1C]

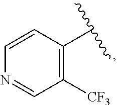 [SMILES: *c1ccncc1C(F)(F)F]

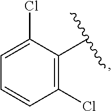 [SMILES: *c1c(Cl)cccc1Cl]

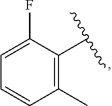 [SMILES: *c1c(C)cccc1F]

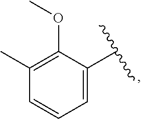 [SMILES: *c1cccc(C)c1OC]

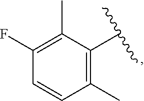 [SMILES: *c1c(C)ccc(F)c1C]

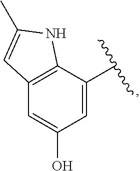 [SMILES: *c1cc(O)cc2cc(C)[nH]c12]

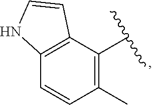 [SMILES: *c1c(C)ccc2[nH]ccc12]

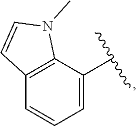 [SMILES: *c1cccc2ccn(C)c12]

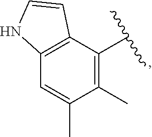 [SMILES: *c1c(C)c(C)cc2[nH]ccc12]

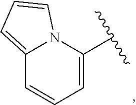 [SMILES: *c1cccc2cccn12]

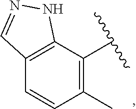 [SMILES: *c1c(C)ccc2cn[nH]c12]

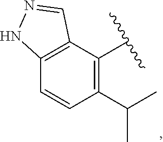 [SMILES: *c1c(C(C)C)ccc2[nH]ncc12]

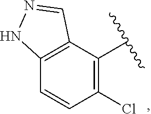 [SMILES: *c1c(Cl)ccc2[nH]ncc12]

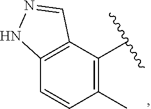 [SMILES: *c1c(C)ccc2[nH]ncc12]

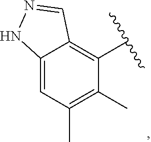 [SMILES: *c1c(C)c(C)cc2[nH]ncc12]

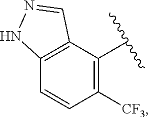 [SMILES: *c1c(C(F)(F)F)ccc2[nH]ncc12]

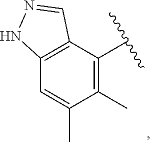 [SMILES: *c1c(C)c(C)cc2[nH]ncc12]

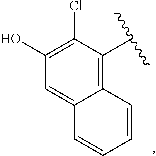 [SMILES: *c1c(Cl)c(O)cc2ccccc12]

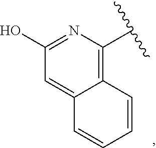 [SMILES: *c1nc(O)cc2ccccc12]

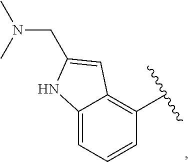 [SMILES: *c1cccc2[nH]c(CN(C)C)cc12]

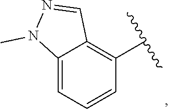 [SMILES: *c1cccc2c1cnn2C]

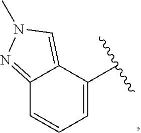 [SMILES: *c1cccc2nn(C)cc12]

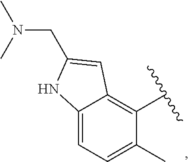

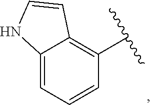 [SMILES: *c1cccc2[nH]ccc12]

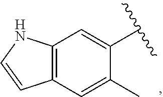 [SMILES: *c1cc2[nH]ccc2cc1C]

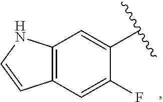 [SMILES: *c1cc2[nH]ccc2cc1F]

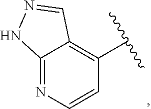 [SMILES: *c1ccnc2[nH]ncc12]

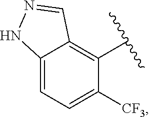 [SMILES: *c1c(C(F)(F)F)ccc2[nH]ncc12]

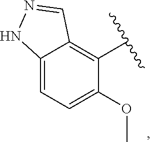 [SMILES: *c1c(OC)ccc2[nH]ncc12]

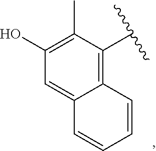 [SMILES: *c1c(C)c(O)cc2ccccc12]

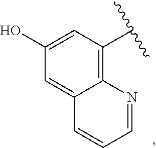 [SMILES: *c1cc(O)cc2cccnc12]

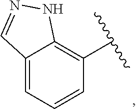 [SMILES: *c1cccc2cn[nH]c12]

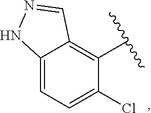 [SMILES: *c1c(Cl)ccc2[nH]ncc12]

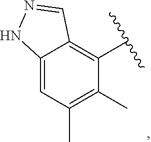 [SMILES: *c1c(C)c(C)cc2[nH]ncc12]

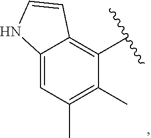 [SMILES: *c1c(C)c(C)cc2[nH]ccc12]

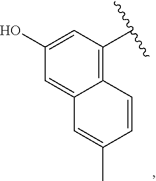 [SMILES: *c1cc(O)cc2cc(C)ccc12]

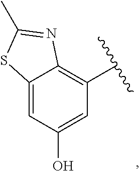 [SMILES: *c1cc(O)cc2sc(C)nc12]

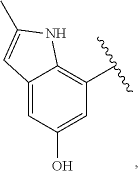 [SMILES: *c1cc(O)cc2cc(C)[nH]c12]

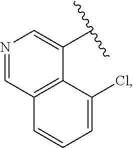 [SMILES: *c1cncc2cccc(Cl)c12]

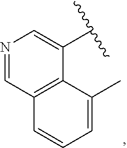 [SMILES: *c1cncc2cccc(C)c12]

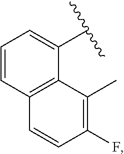 [SMILES: *c1cccc2ccc(F)c(C)c12]

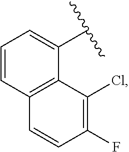 [SMILES: *c1cccc2ccc(F)c(Cl)c12]

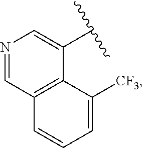 [SMILES: *c1cncc2cccc(C(F)(F)F)c12]

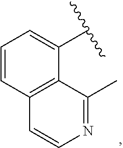 [SMILES: *c1cccc2ccnc(C)c12]

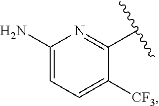 [SMILES: *c1nc(N)ccc1C(F)(F)F]

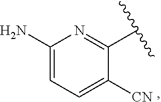 [SMILES: *c1nc(N)ccc1C#N]

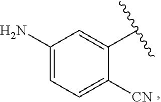 [SMILES: *c1cc(N)ccc1C#N]

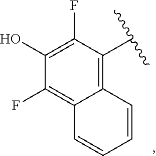 [SMILES: *c1c(F)c(O)c(F)c2ccccc12]

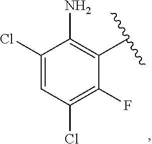 [SMILES: *c1c(N)c(Cl)cc(Cl)c1F]

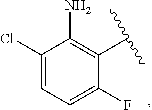 [SMILES: *c1c(F)ccc(Cl)c1N]

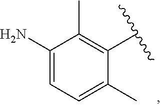 [SMILES: *c1c(C)ccc(N)c1C]

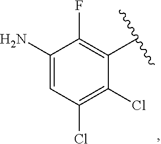 [SMILES: *c1c(F)c(N)cc(Cl)c1Cl]

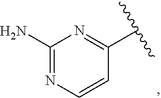 [SMILES: *c1ccnc(N)n1]

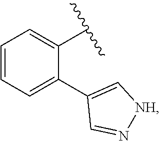 [SMILES: *c1ccccc1-c1cn[nH]c1]

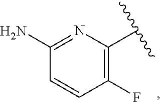 [SMILES: *c1nc(N)ccc1F]

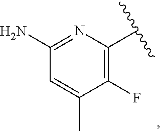 [SMILES: *c1nc(N)cc(C)c1F]

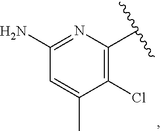 [SMILES: *c1nc(N)cc(C)c1Cl]

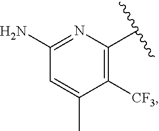 [SMILES: *c1nc(N)cc(C)c1C(F)(F)F]

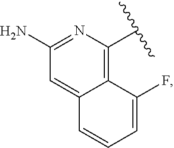 [SMILES: *c1nc(N)cc2cccc(F)c12]

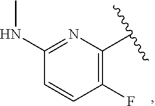 [SMILES: *c1nc(NC)ccc1F]

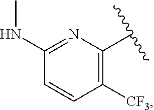 [SMILES: *c1nc(NC)ccc1C(F)(F)F]

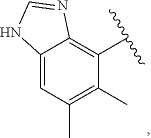 [SMILES: *c1c(C)c(C)cc2[nH]cnc12]

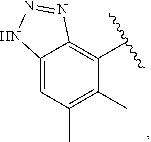 [SMILES: *c1c(C)c(C)cc2[nH]nnc12]

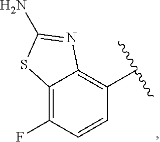 [SMILES: *c1ccc(F)c2sc(N)nc12]

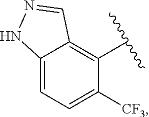 [SMILES: *c1c(C(F)(F)F)ccc2[nH]ncc12]

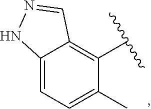 [SMILES: *c1c(C)ccc2[nH]ncc12]

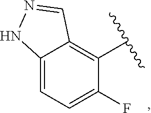 [SMILES: *c1c(F)ccc2[nH]ncc12]

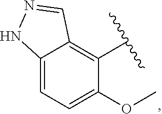 [SMILES: *c1c(OC)ccc2[nH]ncc12]

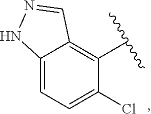 [SMILES: *c1c(Cl)ccc2[nH]ncc12]

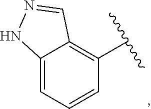 [SMILES: *c1cccc2[nH]ncc12]

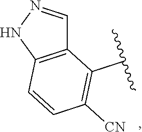 [SMILES: *c1c(C#N)ccc2[nH]ncc12]

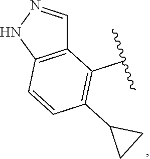 [SMILES: *c1c(C2CC2)ccc2[nH]ncc12]

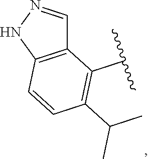 [SMILES: *c1c(C(C)C)ccc2[nH]ncc12]

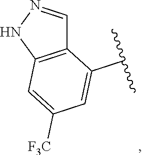 [SMILES: *c1cc(C(F)(F)F)cc2[nH]ncc12]

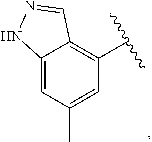 [SMILES: *c1cc(C)cc2[nH]ncc12]

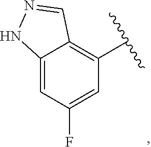 [SMILES: *c1cc(F)cc2[nH]ncc12]

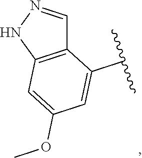 [SMILES: *c1cc(OC)cc2[nH]ncc12]

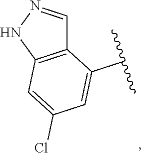 [SMILES: *c1cc(Cl)cc2[nH]ncc12]

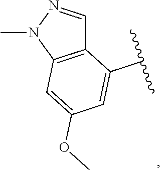 [SMILES: *c1cc(OC)cc2c1cnn2C]

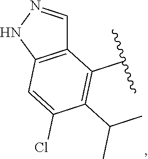 [SMILES: *c1c(C(C)C)c(Cl)cc2[nH]ncc12]

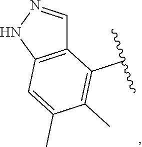 [SMILES: *c1c(C)c(C)cc2[nH]ncc12]

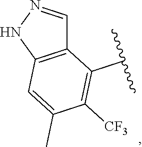 [SMILES: *c1c(C(F)(F)F)c(C)cc2[nH]ncc12]

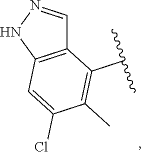 [SMILES: *c1c(C)c(Cl)cc2[nH]ncc12]

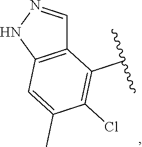 [SMILES: *c1c(Cl)c(C)cc2[nH]ncc12]

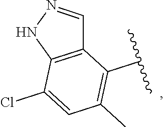 [SMILES: *c1c(C)cc(Cl)c2[nH]ncc12]

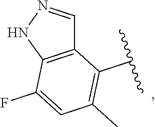 [SMILES: *c1c(C)cc(F)c2[nH]ncc12]

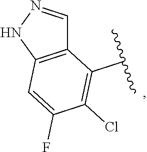 [SMILES: *c1c(Cl)c(F)cc2[nH]ncc12]

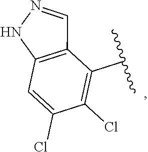 [SMILES: *c1c(Cl)c(Cl)cc2[nH]ncc12]

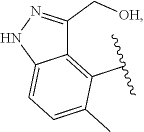 [SMILES: *c1c(C)ccc2[nH]nc(CO)c12]

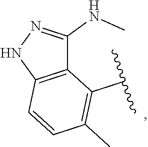 [SMILES: *c1c(C)ccc2[nH]nc(NC)c12]

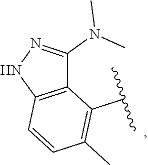 [SMILES: *c1c(C)ccc2[nH]nc(N(C)C)c12]

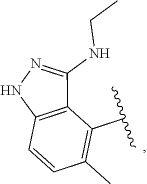 [SMILES: *c1c(C)ccc2[nH]nc(NCC)c12]

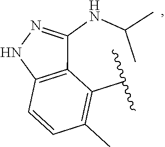 [SMILES: *c1c(C)ccc2[nH]nc(NC(C)C)c12]

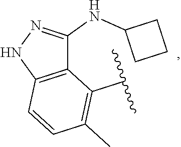 [SMILES: *c1c(C)ccc2[nH]nc(NC3CCC3)c12]

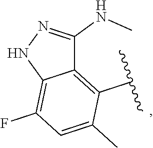 [SMILES: *c1c(C)cc(F)c2[nH]nc(NC)c12]

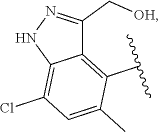 [SMILES: *c1c(C)cc(Cl)c2[nH]nc(CO)c12]

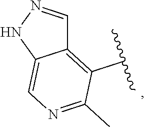 [SMILES: *c1c(C)ncc2[nH]ncc12]

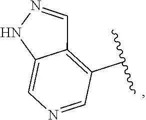 [SMILES: *c1cncc2[nH]ncc12]

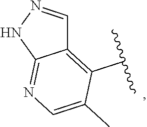 [SMILES: *c1c(C)cnc2[nH]ncc12]

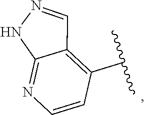 [SMILES: *c1ccnc2[nH]ncc12]

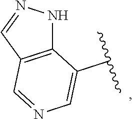 [SMILES: *c1cncc2cn[nH]c12]

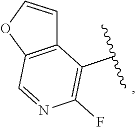 [SMILES: *c1c(F)ncc2occc12]

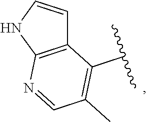 [SMILES: *c1c(C)cnc2[nH]ccc12]

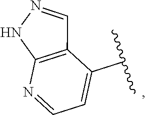 [SMILES: *c1ccnc2[nH]ncc12]

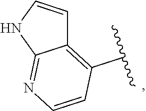 [SMILES: *c1ccnc2[nH]ccc12]

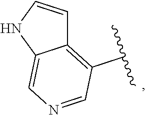 [SMILES: *c1cncc2[nH]ccc12]

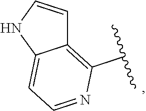 [SMILES: *c1nccc2[nH]ccc12]

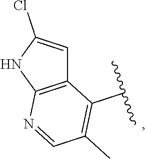 [SMILES: *c1c(C)cnc2[nH]c(Cl)cc12]

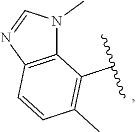 [SMILES: *c1c(C)ccc2ncn(C)c12]

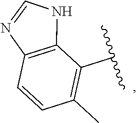 [SMILES: *c1c(C)ccc2nc[nH]c12]

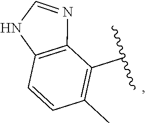 [SMILES: *c1c(C)ccc2[nH]cnc12]

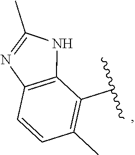 [SMILES: *c1c(C)ccc2nc(C)[nH]c12]

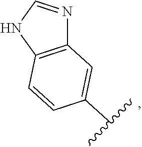 [SMILES: *c1ccc2[nH]cnc2c1]

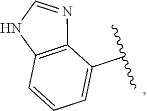 [SMILES: *c1cccc2[nH]cnc12]

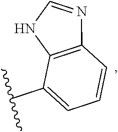 [SMILES: *c1cccc2nc[nH]c12]

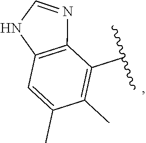 [SMILES: *c1c(C)c(C)cc2[nH]cnc12]

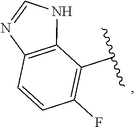 [SMILES: *c1c(F)ccc2nc[nH]c12]

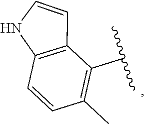 [SMILES: *c1c(C)ccc2[nH]ccc12]

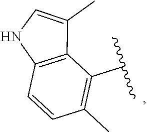 [SMILES: *c1c(C)ccc2[nH]cc(C)c12]

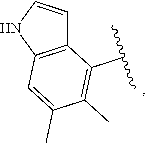 [SMILES: *c1c(C)c(C)cc2[nH]ccc12]

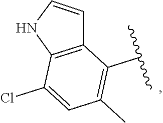 [SMILES: *c1c(C)cc(Cl)c2[nH]ccc12]

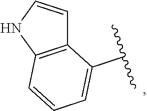 [SMILES: *c1cccc2[nH]ccc12]

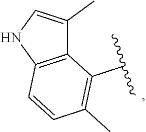 [SMILES: *c1c(C)ccc2[nH]cc(C)c12]

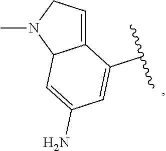 [SMILES: *C1=CC(N)=CC2C1=CCN2C]

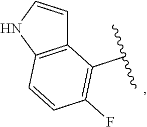 [SMILES: *c1c(F)ccc2[nH]ccc12]

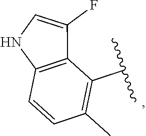 [SMILES: *c1c(C)ccc2[nH]cc(F)c12]

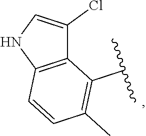 [SMILES: *c1c(C)ccc2[nH]cc(Cl)c12]

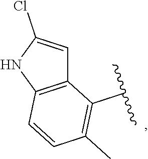 [SMILES: *c1c(C)ccc2[nH]c(Cl)cc12]

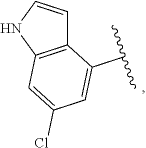 [SMILES: *c1cc(Cl)cc2[nH]ccc12]

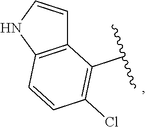 [SMILES: *c1c(Cl)ccc2[nH]ccc12]

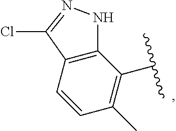 [SMILES: *c1c(C)ccc2c(Cl)n[nH]c12]

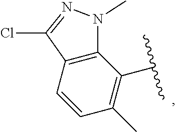 [SMILES: *c1c(C)ccc2c(Cl)nn(C)c12]

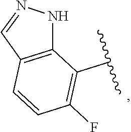 [SMILES: *c1c(F)ccc2cn[nH]c12]

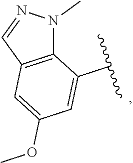 [SMILES: *c1cc(OC)cc2cnn(C)c12]

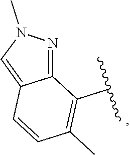 [SMILES: *c1c(C)ccc2cn(C)nc12]

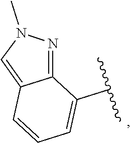 [SMILES: *c1cccc2cn(C)nc12]

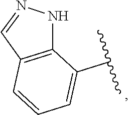 [SMILES: *c1cccc2cn[nH]c12]

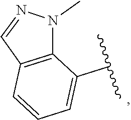 [SMILES: *c1cccc2cnn(C)c12]

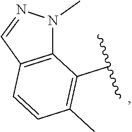 [SMILES: *c1c(C)ccc2cnn(C)c12]

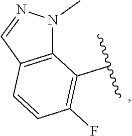 [SMILES: *c1c(F)ccc2cnn(C)c12]

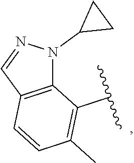 [SMILES: *c1c(C)ccc2cnn(C3CC3)c12]

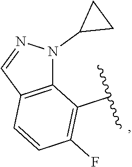 [SMILES: *c1c(F)ccc2cnn(C3CC3)c12]

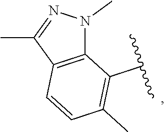 [SMILES: *c1c(C)ccc2c(C)nn(C)c12]

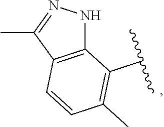 [SMILES: *c1c(C)ccc2c(C)n[nH]c12]

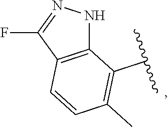 [SMILES: *c1c(C)ccc2c(F)n[nH]c12]

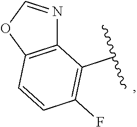 [SMILES: *c1c(F)ccc2ocnc12]

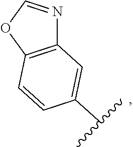 [SMILES: *c1ccc2ocnc2c1]

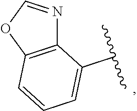 [SMILES: *c1cccc2ocnc12]

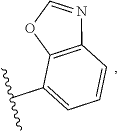 [SMILES: *c1cccc2ncoc12]

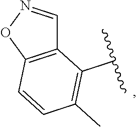 [SMILES: *c1c(C)ccc2oncc12]

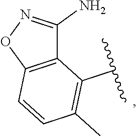 [SMILES: *c1c(C)ccc2onc(N)c12]

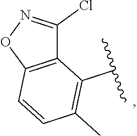 [SMILES: *c1c(C)ccc2onc(Cl)c12]

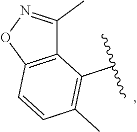 [SMILES: *c1c(C)ccc2onc(C)c12]

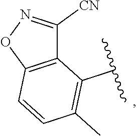 [SMILES: *c1c(C)ccc2onc(C#N)c12]

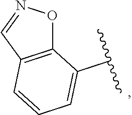 [SMILES: *c1cccc2cnoc12]

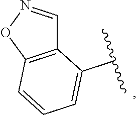 [SMILES: *c1cccc2oncc12]

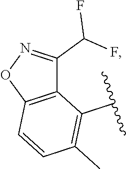 [SMILES: *c1c(C)ccc2onc(C(F)F)c12]

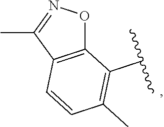 [SMILES: *c1c(C)ccc2c(C)noc12]

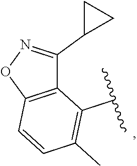 [SMILES: *c1c(C)ccc2onc(C3CC3)c12]

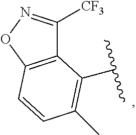 [SMILES: *c1c(C)ccc2onc(C(F)(F)F)c12]

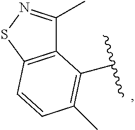 [SMILES: *c1c(C)ccc2snc(C)c12]

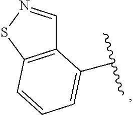 [SMILES: *c1cccc2sncc12]

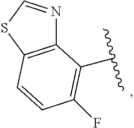 [SMILES: *c1c(F)ccc2scnc12]

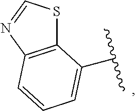 [SMILES: *c1cccc2ncsc12]

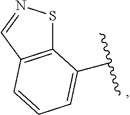 [SMILES: *c1cccc2cnsc12]

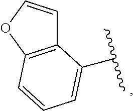 [SMILES: *c1cccc2occc12]

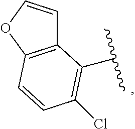 [SMILES: *c1c(Cl)ccc2occc12]

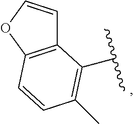 [SMILES: *c1c(C)ccc2occc12]

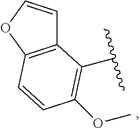 [SMILES: *c1c(OC)ccc2occc12]

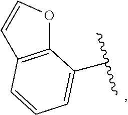 [SMILES: *c1cccc2ccoc12]

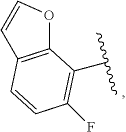 [SMILES: *c1c(F)ccc2ccoc12]

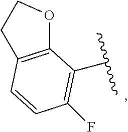 [SMILES: *c1c(F)ccc2c1OCC2]

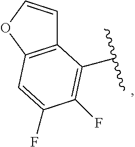 [SMILES: *c1c(F)c(F)cc2occc12]

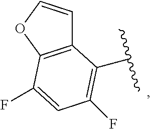 [SMILES: *c1c(F)cc(F)c2occc12]

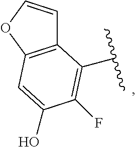 [SMILES: *c1c(F)c(O)cc2occc12]

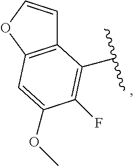 [SMILES: *c1c(F)c(OC)cc2occc12]

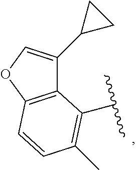 [SMILES: *c1c(C)ccc2occ(C3CC3)c12]

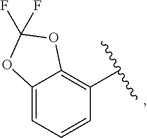 [SMILES: *c1cccc2c1OC(F)(F)O2]

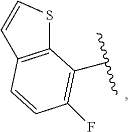 [SMILES: *c1c(F)ccc2ccsc12]

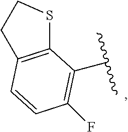 [SMILES: *c1c(F)ccc2c1SCC2]

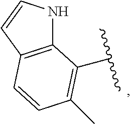 [SMILES: *c1c(C)ccc2cc[nH]c12]

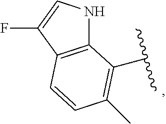 [SMILES: *c1c(C)ccc2c(F)c[nH]c12]

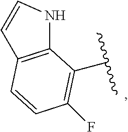 [SMILES: *c1c(F)ccc2cc[nH]c12]

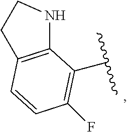 [SMILES: *c1c(F)ccc2c1NCC2]

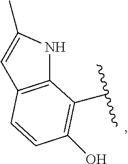 [SMILES: *c1c(O)ccc2cc(C)[nH]c12]

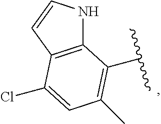 [SMILES: *c1c(C)cc(Cl)c2cc[nH]c12]

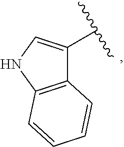 [SMILES: *c1c[nH]c2ccccc12]

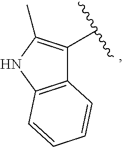 [SMILES: *c1c(C)[nH]c2ccccc12]

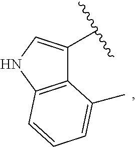 [SMILES: *c1c[nH]c2cccc(C)c12]

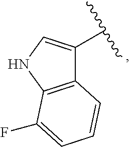 [SMILES: *c1c[nH]c2c(F)cccc12]

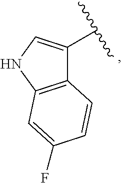 [SMILES: *c1c[nH]c2cc(F)ccc12]

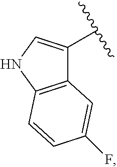 [SMILES: *c1c[nH]c2ccc(F)cc12]

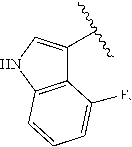 [SMILES: *c1c[nH]c2cccc(F)c12]

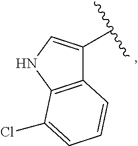 [SMILES: *c1c[nH]c2c(Cl)cccc12]

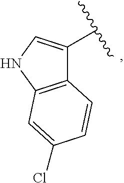 [SMILES: *c1c[nH]c2cc(Cl)ccc12]

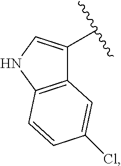 [SMILES: *c1c[nH]c2ccc(Cl)cc12]

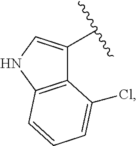 [SMILES: *c1c[nH]c2cccc(Cl)c12]

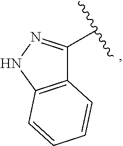 [SMILES: *c1n[nH]c2ccccc12]

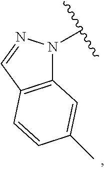 [SMILES: *n1ncc2ccc(C)cc21]

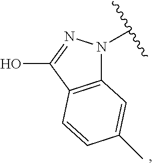 [SMILES: *n1nc(O)c2ccc(C)cc21]

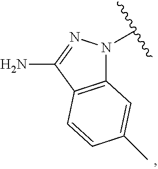 [SMILES: *n1nc(N)c2ccc(C)cc21]

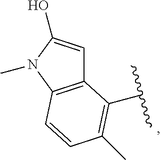 [SMILES: *c1c(C)ccc2c1cc(O)n2C]

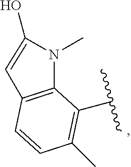 [SMILES: *c1c(C)ccc2cc(O)n(C)c12]

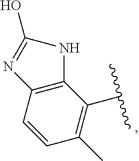 [SMILES: *c1c(C)ccc2nc(O)[nH]c12]

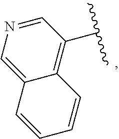 [SMILES: *c1cncc2ccccc12]

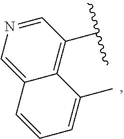 [SMILES: *c1cncc2cccc(C)c12]

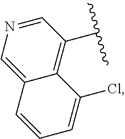 [SMILES: *c1cncc2cccc(Cl)c12]

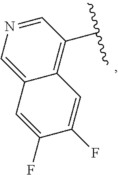 [SMILES: *c1cncc2cc(F)c(F)cc12]

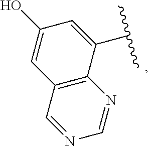 [SMILES: *c1cc(O)cc2cncnc12]

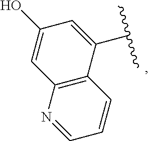 [SMILES: *c1cc(O)cc2ncccc12]

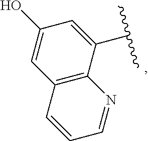 [SMILES: *c1cc(O)cc2cccnc12]

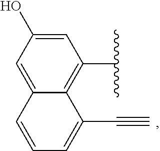 [SMILES: *c1cc(O)cc2cccc(C#C)c12]

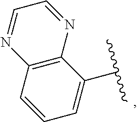 [SMILES: *c1cccc2nccnc12]

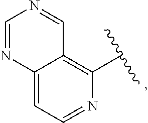 [SMILES: *c1nccc2ncncc12]

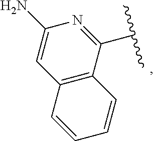 [SMILES: *c1nc(N)cc2ccccc12]

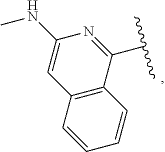 [SMILES: *c1nc(NC)cc2ccccc12]

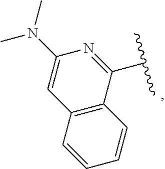 [SMILES: *c1nc(N(C)C)cc2ccccc12]

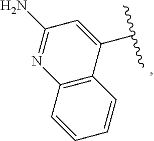 [SMILES: *c1cc(N)nc2ccccc12]

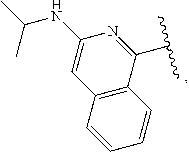 [SMILES: *c1nc(NC(C)C)cc2ccccc12]

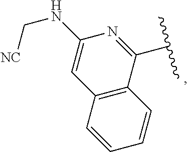 [SMILES: *c1nc(NCC#N)cc2ccccc12]

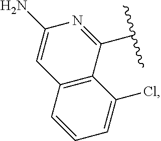 [SMILES: *c1nc(N)cc2cccc(Cl)c12]

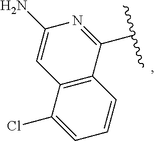 [SMILES: *c1nc(N)cc2c(Cl)cccc12]

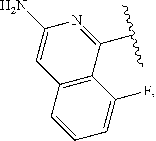 [SMILES: *c1nc(N)cc2cccc(F)c12]

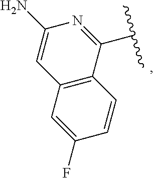 [SMILES: *c1nc(N)cc2cc(F)ccc12]

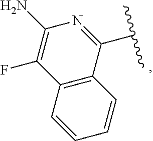 [SMILES: *c1nc(N)c(F)c2ccccc12]

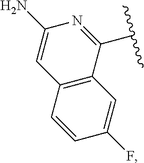 [SMILES: *c1nc(N)cc2ccc(F)cc12]

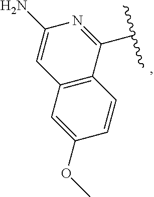 [SMILES: *c1nc(N)cc2cc(OC)ccc12]

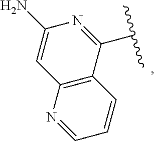 [SMILES: *c1nc(N)cc2ncccc12]

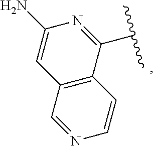 [SMILES: *c1nc(N)cc2cnccc12]

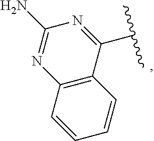 [SMILES: *c1nc(N)nc2ccccc12]

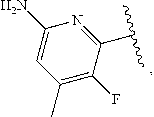 [SMILES: *c1nc(N)cc(C)c1F]

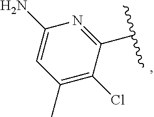 [SMILES: *c1nc(N)cc(C)c1Cl]

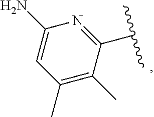 [SMILES: *c1nc(N)cc(C)c1C]

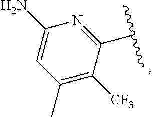 [SMILES: *c1nc(N)cc(C)c1C(F)(F)F]

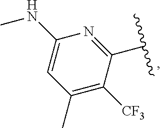 [SMILES: *c1nc(NC)cc(C)c1C(F)(F)F]

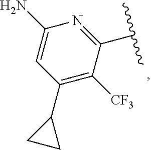 [SMILES: *c1nc(N)cc(C2CC2)c1C(F)(F)F]

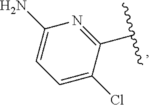 [SMILES: *c1nc(N)ccc1Cl]

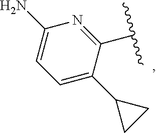 [SMILES: *c1nc(N)ccc1C1CC1]

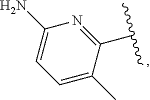 [SMILES: *c1nc(N)ccc1C]

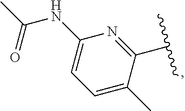 [SMILES: *c1nc(NC(C)=O)ccc1C]

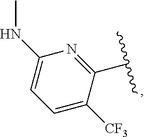 [SMILES: *c1nc(NC)ccc1C(F)(F)F]

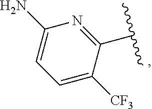 [SMILES: *c1nc(N)ccc1C(F)(F)F]

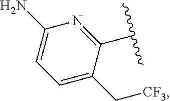 [SMILES: *c1nc(N)ccc1CC(F)(F)F]

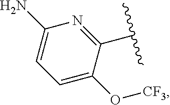 [SMILES: *c1nc(N)ccc1OC(F)(F)F]

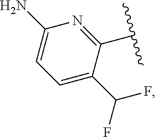 [SMILES: *c1nc(N)ccc1C(F)F]

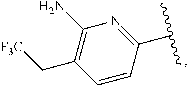 [SMILES: *c1ccc(CC(F)(F)F)c(N)n1]

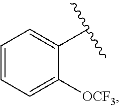 [SMILES: *c1ccccc1OC(F)(F)F]

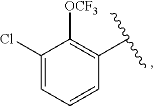 [SMILES: *c1cccc(Cl)c1OC(F)(F)F]

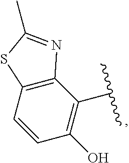 [SMILES: *c1c(O)ccc2sc(C)nc12]

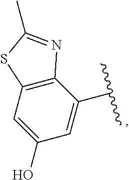 [SMILES: *c1cc(O)cc2sc(C)nc12]

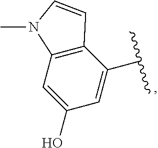 [SMILES: *c1cc(O)cc2c1ccn2C]

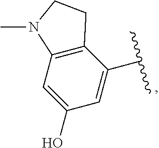 [SMILES: *c1cc(O)cc2c1CCN2C]

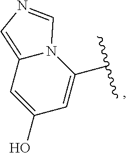 [SMILES: *c1cc(O)cc2cncn12]

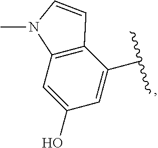 [SMILES: *c1cc(O)cc2c1ccn2C]

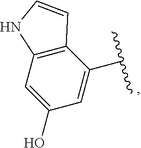 [SMILES: *c1cc(O)cc2[nH]ccc12]

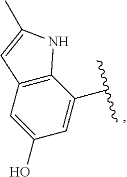 [SMILES: *c1cc(O)cc2cc(C)[nH]c12]

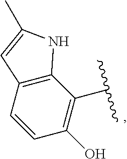 [SMILES: *c1c(O)ccc2cc(C)[nH]c12]

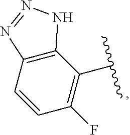 [SMILES: *c1c(F)ccc2nn[nH]c12]

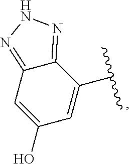 [SMILES: *c1cc(O)cc2n[nH]nc12]

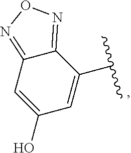 [SMILES: *c1cc(O)cc2nonc12]

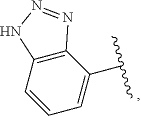 [SMILES: *c1cccc2[nH]nnc12]

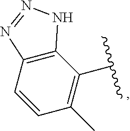 [SMILES: *c1c(C)ccc2nn[nH]c12]

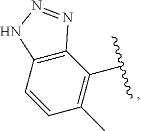 [SMILES: *c1c(C)ccc2[nH]nnc12]

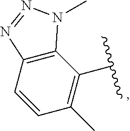 [SMILES: *c1c(C)ccc2nnn(C)c12]

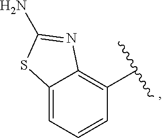 [SMILES: *c1cccc2sc(N)nc12]

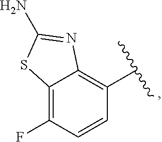 [SMILES: *c1ccc(F)c2sc(N)nc12]

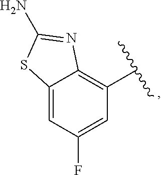 [SMILES: *c1cc(F)cc2sc(N)nc12]

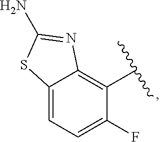 [SMILES: *c1c(F)ccc2sc(N)nc12]

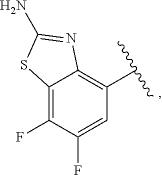 [SMILES: *c1cc(F)c(F)c2sc(N)nc12]

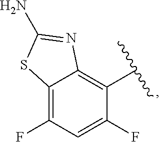 [SMILES: *c1c(F)cc(F)c2sc(N)nc12]

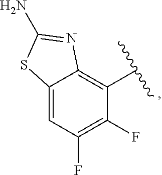 [SMILES: *c1c(F)c(F)cc2sc(N)nc12]

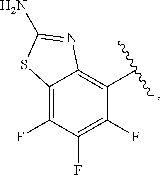 [SMILES: *c1c(F)c(F)c(F)c2sc(N)nc12]

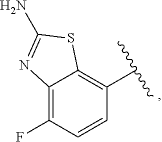 [SMILES: *c1ccc(F)c2nc(N)sc12]

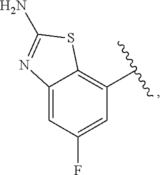 [SMILES: *c1cc(F)cc2nc(N)sc12]

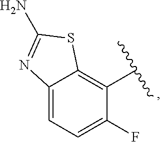 [SMILES: *c1c(F)ccc2nc(N)sc12]

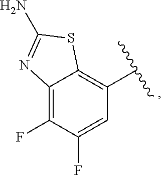 [SMILES: *c1cc(F)c(F)c2nc(N)sc12]

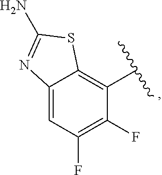 [SMILES: *c1c(F)c(F)cc2nc(N)sc12]

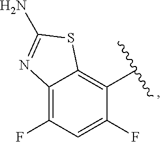 [SMILES: *c1c(F)cc(F)c2nc(N)sc12]

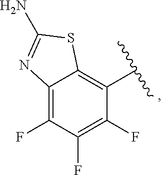 [SMILES: *c1c(F)c(F)c(F)c2nc(N)sc12]

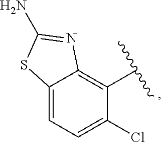 [SMILES: *c1c(Cl)ccc2sc(N)nc12]

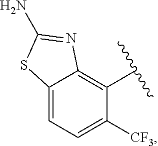 [SMILES: *c1c(C(F)(F)F)ccc2sc(N)nc12]

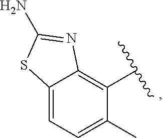 [SMILES: *c1c(C)ccc2sc(N)nc12]

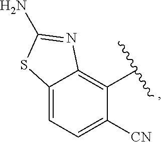 [SMILES: *c1c(C#N)ccc2sc(N)nc12]

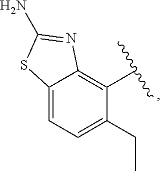 [SMILES: *c1c(CC)ccc2sc(N)nc12]

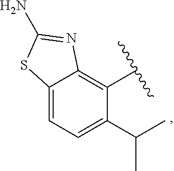 [SMILES: *c1c(C(C)C)ccc2sc(N)nc12]

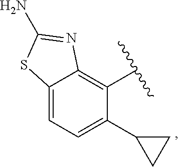 [SMILES: *c1c(C2CC2)ccc2sc(N)nc12]

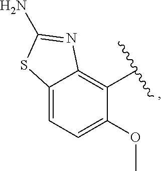 [SMILES: *c1c(OC)ccc2sc(N)nc12]

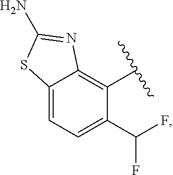 [SMILES: *c1c(C(F)F)ccc2sc(N)nc12]

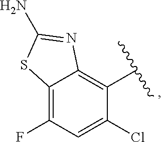 [SMILES: *c1c(Cl)cc(F)c2sc(N)nc12]

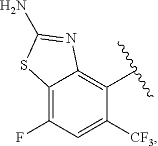 [SMILES: *c1c(C(F)(F)F)cc(F)c2sc(N)nc12]

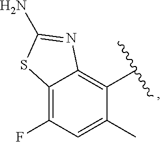 [SMILES: *c1c(C)cc(F)c2sc(N)nc12]

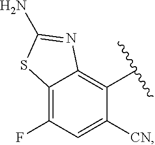 [SMILES: *c1c(C#N)cc(F)c2sc(N)nc12]

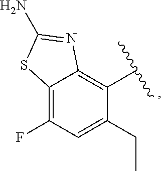 [SMILES: *c1c(CC)cc(F)c2sc(N)nc12]

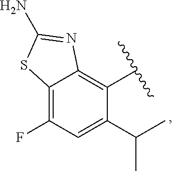 [SMILES: *c1c(C(C)C)cc(F)c2sc(N)nc12]

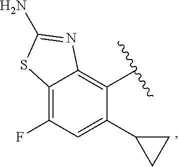 [SMILES: *c1c(C2CC2)cc(F)c2sc(N)nc12]

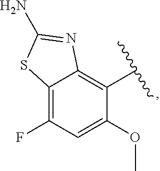 [SMILES: *c1c(OC)cc(F)c2sc(N)nc12]

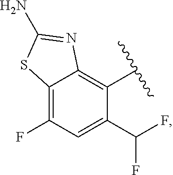 [SMILES: *c1c(C(F)F)cc(F)c2sc(N)nc12]

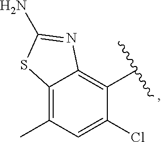 [SMILES: *c1c(Cl)cc(C)c2sc(N)nc12]

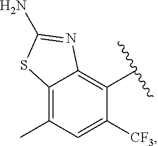 [SMILES: *c1c(C(F)(F)F)cc(C)c2sc(N)nc12]

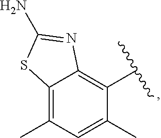

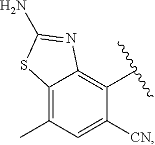 [SMILES: *c1c(C#N)cc(C)c2sc(N)nc12]

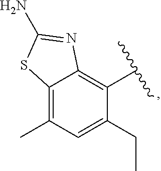 [SMILES: *c1c(CC)cc(C)c2sc(N)nc12]

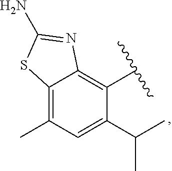 [SMILES: *c1c(C(C)C)cc(C)c2sc(N)nc12]

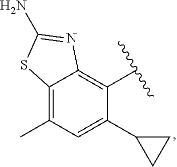 [SMILES: *c1c(C2CC2)cc(C)c2sc(N)nc12]

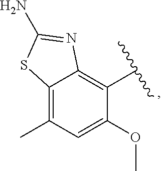

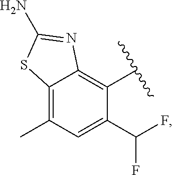 [SMILES: *c1c(C(F)F)cc(C)c2sc(N)nc12]

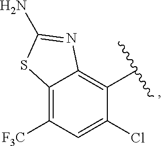 [SMILES: *c1c(Cl)cc(C(F)(F)F)c2sc(N)nc12]

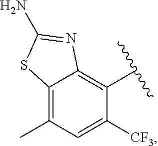 [SMILES: *c1c(C(F)(F)F)cc(C)c2sc(N)nc12]

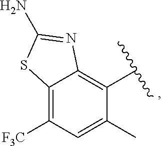 [SMILES: *c1c(C)cc(C(F)(F)F)c2sc(N)nc12]

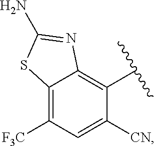 [SMILES: *c1c(C#N)cc(C(F)(F)F)c2sc(N)nc12]

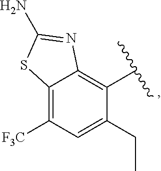 [SMILES: *c1c(CC)cc(C(F)(F)F)c2sc(N)nc12]

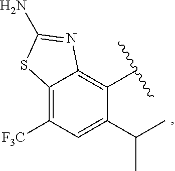 [SMILES: *c1c(C(C)C)cc(C(F)(F)F)c2sc(N)nc12]

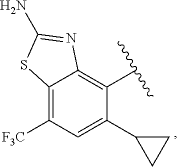 [SMILES: *c1c(C2CC2)cc(C(F)(F)F)c2sc(N)nc12]

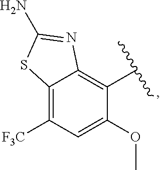 [SMILES: *c1c(OC)cc(C(F)(F)F)c2sc(N)nc12]

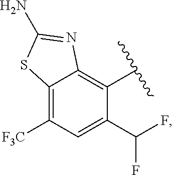 [SMILES: *c1c(C(F)F)cc(C(F)(F)F)c2sc(N)nc12]

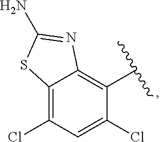 [SMILES: *c1c(Cl)cc(Cl)c2sc(N)nc12]

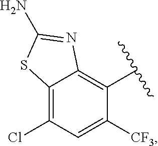 [SMILES: *c1c(C(F)(F)F)cc(Cl)c2sc(N)nc12]

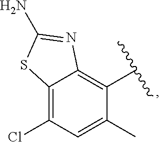 [SMILES: *c1c(C)cc(Cl)c2sc(N)nc12]

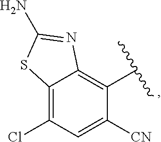 [SMILES: *c1c(C#N)cc(Cl)c2sc(N)nc12]

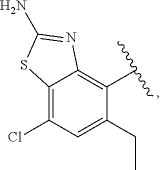 [SMILES: *c1c(CC)cc(Cl)c2sc(N)nc12]

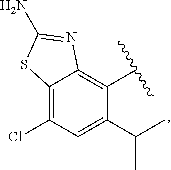 [SMILES: *c1c(C(C)C)cc(Cl)c2sc(N)nc12]

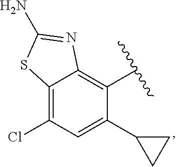 [SMILES: *c1c(C2CC2)cc(Cl)c2sc(N)nc12]

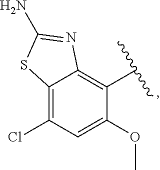 [SMILES: *c1c(OC)cc(Cl)c2sc(N)nc12]

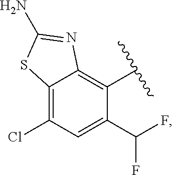 [SMILES: *c1c(C(F)F)cc(Cl)c2sc(N)nc12]

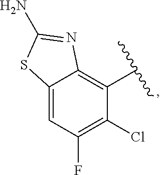 [SMILES: *c1c(Cl)c(F)cc2sc(N)nc12]

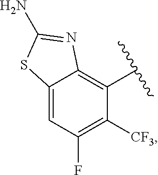 [SMILES: *c1c(C(F)(F)F)c(F)cc2sc(N)nc12]

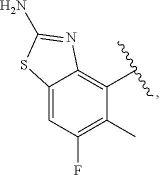 [SMILES: *c1c(C)c(F)cc2sc(N)nc12]

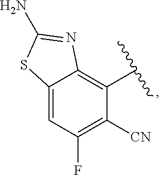 [SMILES: *c1c(C#N)c(F)cc2sc(N)nc12]

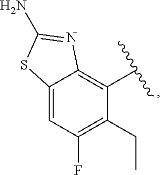 [SMILES: *c1c(CC)c(F)cc2sc(N)nc12]

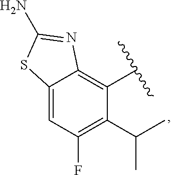 [SMILES: *c1c(C(C)C)c(F)cc2sc(N)nc12]

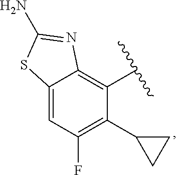 [SMILES: *c1c(C2CC2)c(F)cc2sc(N)nc12]

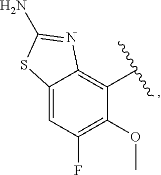 [SMILES: *c1c(OC)c(F)cc2sc(N)nc12]

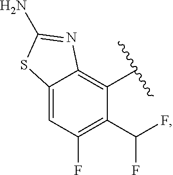 [SMILES: *c1c(C(F)F)c(F)cc2sc(N)nc12]

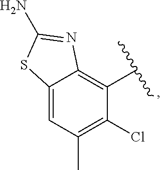 [SMILES: *c1c(Cl)c(C)cc2sc(N)nc12]

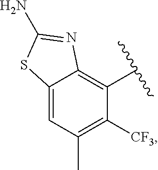 [SMILES: *c1c(C(F)(F)F)c(C)cc2sc(N)nc12]

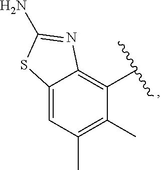 [SMILES: *c1c(C)c(C)cc2sc(N)nc12]

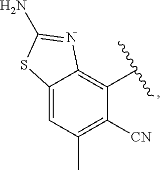 [SMILES: *c1c(C#N)c(C)cc2sc(N)nc12]

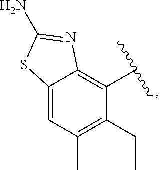 [SMILES: *c1c(CC)c(C)cc2sc(N)nc12]

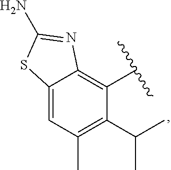 [SMILES: *c1c(C(C)C)c(C)cc2sc(N)nc12]

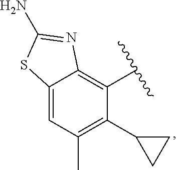 [SMILES: *c1c(C2CC2)c(C)cc2sc(N)nc12]

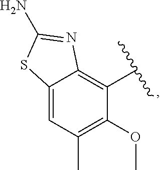 [SMILES: *c1c(OC)c(C)cc2sc(N)nc12]

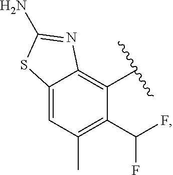 [SMILES: *c1c(C(F)F)c(C)cc2sc(N)nc12]

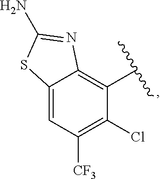 [SMILES: *c1c(Cl)c(C(F)(F)F)cc2sc(N)nc12]

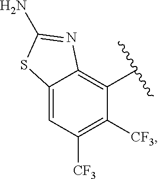 [SMILES: *c1c(C(F)(F)F)c(C(F)(F)F)cc2sc(N)nc12]

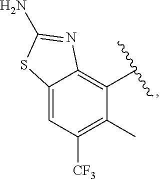 [SMILES: *c1c(C)c(C(F)(F)F)cc2sc(N)nc12]

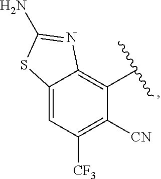 [SMILES: *c1c(C#N)c(C(F)(F)F)cc2sc(N)nc12]

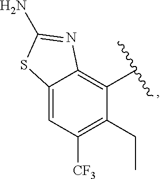 [SMILES: *c1c(CC)c(C(F)(F)F)cc2sc(N)nc12]

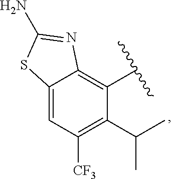 [SMILES: *c1c(C(C)C)c(C(F)(F)F)cc2sc(N)nc12]

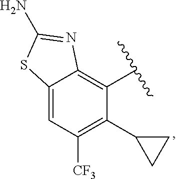 [SMILES: *c1c(C2CC2)c(C(F)(F)F)cc2sc(N)nc12]

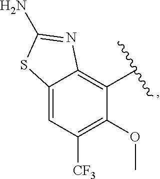 [SMILES: *c1c(OC)c(C(F)(F)F)cc2sc(N)nc12]

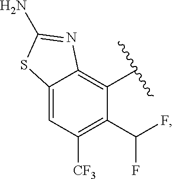 [SMILES: *c1c(C(F)F)c(C(F)(F)F)cc2sc(N)nc12]

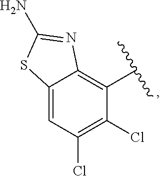 [SMILES: *c1c(Cl)c(Cl)cc2sc(N)nc12]

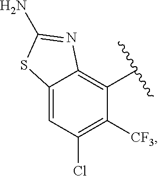 [SMILES: *c1c(C(F)(F)F)c(Cl)cc2sc(N)nc12]

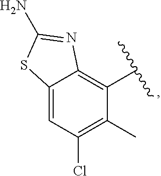 [SMILES: *c1c(C)c(Cl)cc2sc(N)nc12]

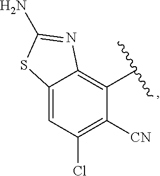 [SMILES: *c1c(C#N)c(Cl)cc2sc(N)nc12]

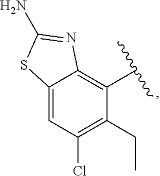 [SMILES: *c1c(CC)c(Cl)cc2sc(N)nc12]

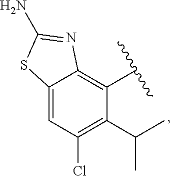 [SMILES: *c1c(C(C)C)c(Cl)cc2sc(N)nc12]

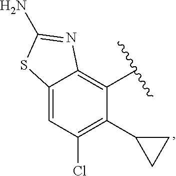 [SMILES: *c1c(C2CC2)c(Cl)cc2sc(N)nc12]

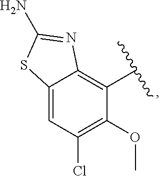 [SMILES: *c1c(OC)c(Cl)cc2sc(N)nc12]

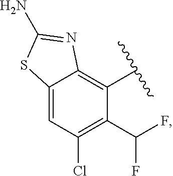 [SMILES: *c1c(C(F)F)c(Cl)cc2sc(N)nc12]

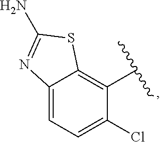 [SMILES: *c1c(Cl)ccc2nc(N)sc12]

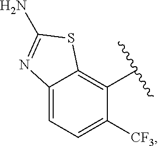 [SMILES: *c1c(C(F)(F)F)ccc2nc(N)sc12]

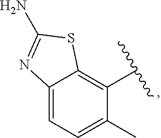 [SMILES: *c1c(C)ccc2nc(N)sc12]

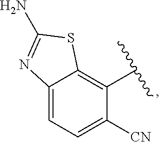 [SMILES: *c1c(C#N)ccc2nc(N)sc12]

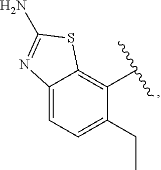 [SMILES: *c1c(CC)ccc2nc(N)sc12]

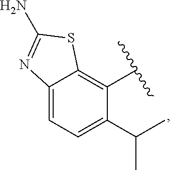 [SMILES: *c1c(C(C)C)ccc2nc(N)sc12]

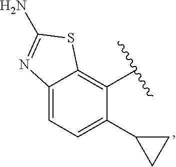 [SMILES: *c1c(C2CC2)ccc2nc(N)sc12]

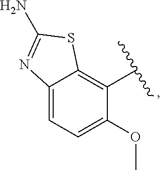 [SMILES: *c1c(OC)ccc2nc(N)sc12]

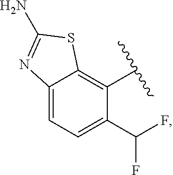 [SMILES: *c1c(C(F)F)ccc2nc(N)sc12]

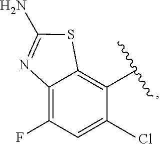 [SMILES: *c1c(C)cc(F)c2nc(N)sc12 ; Cl]

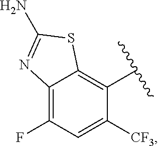 [SMILES: *c1c(C(F)(F)F)cc(F)c2nc(N)sc12]

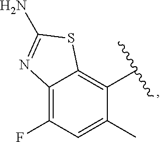 [SMILES: *c1c(C)cc(F)c2nc(N)sc12]

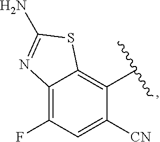 [SMILES: *c1c(C#N)cc(F)c2nc(N)sc12]

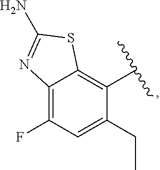 [SMILES: *c1c(CC)cc(F)c2nc(N)sc12]

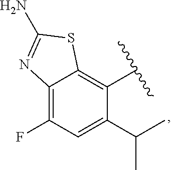 [SMILES: *c1c(C(C)C)cc(F)c2nc(N)sc12]

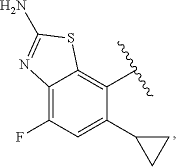 [SMILES: *c1c(C2CC2)cc(F)c2nc(N)sc12]

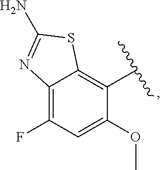 [SMILES: *c1c(OC)cc(F)c2nc(N)sc12]

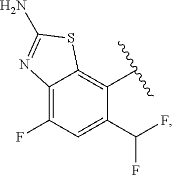 [SMILES: *c1c(C(F)F)cc(F)c2nc(N)sc12]

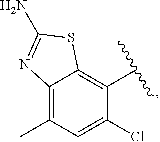 [SMILES: *c1c(Cl)cc(C)c2nc(N)sc12]

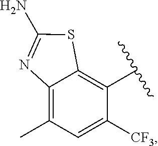 [SMILES: *c1c(C(F)(F)F)cc(C)c2nc(N)sc12]

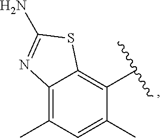

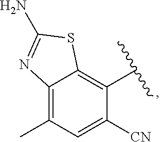 [SMILES: *c1c(C#N)cc(C)c2nc(N)sc12]

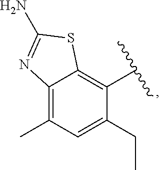 [SMILES: *c1c(CC)cc(C)c2nc(N)sc12]

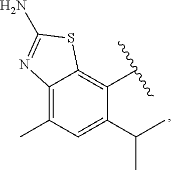 [SMILES: *c1c(C(C)C)cc(C)c2nc(N)sc12]

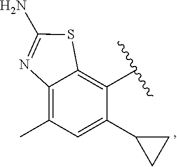 [SMILES: *c1c(C2CC2)cc(C)c2nc(N)sc12]

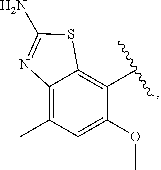

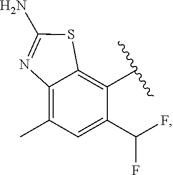 [SMILES: *c1c(C(F)F)cc(C)c2nc(N)sc12]

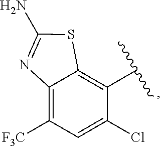 [SMILES: *c1c(Cl)cc(C(F)(F)F)c2nc(N)sc12]

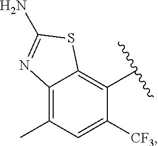 [SMILES: *c1c(C(F)(F)F)cc(C)c2nc(N)sc12]

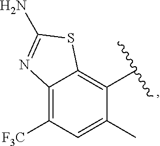 [SMILES: *c1c(C)cc(C(F)(F)F)c2nc(N)sc12]

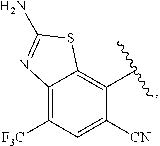 [SMILES: *c1c(C#N)cc(C(F)(F)F)c2nc(N)sc12]

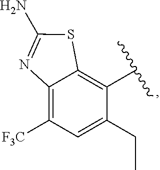 [SMILES: *c1c(CC)cc(C(F)(F)F)c2nc(N)sc12]

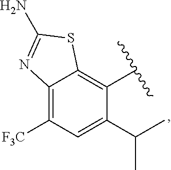 [SMILES: *c1c(C(C)C)cc(C(F)(F)F)c2nc(N)sc12]

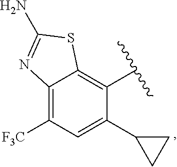 [SMILES: *c1c(C2CC2)cc(C(F)(F)F)c2nc(N)sc12]

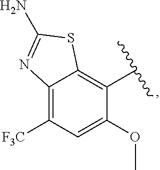 [SMILES: *c1c(OC)cc(C(F)(F)F)c2nc(N)sc12]

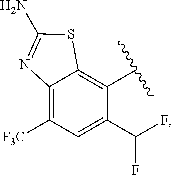 [SMILES: *c1c(C(F)F)cc(C(F)(F)F)c2nc(N)sc12]

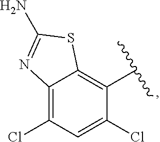 [SMILES: *c1c(Cl)cc(Cl)c2nc(N)sc12]

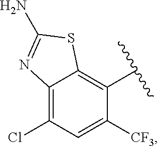 [SMILES: *c1c(C(F)(F)F)cc(Cl)c2nc(N)sc12]

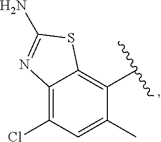 [SMILES: *c1c(C)cc(Cl)c2nc(N)sc12]

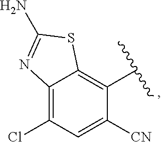 [SMILES: *c1c(C#N)cc(Cl)c2nc(N)sc12]

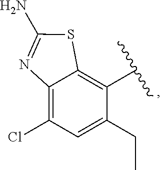 [SMILES: *c1c(CC)cc(Cl)c2nc(N)sc12]

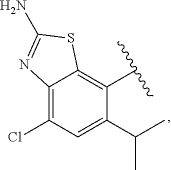 [SMILES: *c1c(C(C)C)cc(Cl)c2nc(N)sc12]

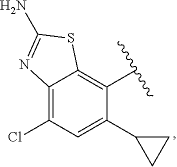 [SMILES: *c1c(C2CC2)cc(Cl)c2nc(N)sc12]

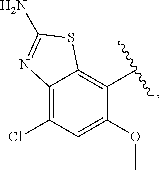 [SMILES: *c1c(OC)cc(Cl)c2nc(N)sc12]

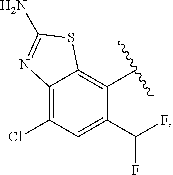 [SMILES: *c1c(C(F)F)cc(Cl)c2nc(N)sc12]

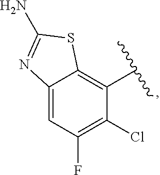 [SMILES: *c1c(Cl)c(F)cc2nc(N)sc12]

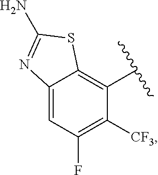 [SMILES: *c1c(C(F)(F)F)c(F)cc2nc(N)sc12]

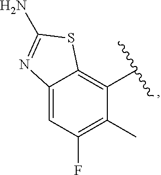 [SMILES: *c1c(C)c(F)cc2nc(N)sc12]

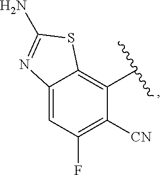 [SMILES: *c1c(C#N)c(F)cc2nc(N)sc12]

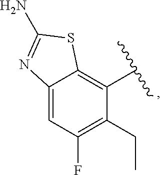 [SMILES: *c1c(CC)c(F)cc2nc(N)sc12]

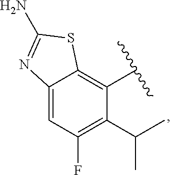 [SMILES: *c1c(C(C)C)c(F)cc2nc(N)sc12]

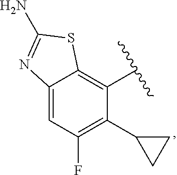 [SMILES: *c1c(C2CC2)c(F)cc2nc(N)sc12]

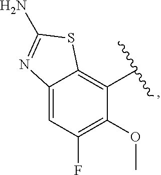 [SMILES: *c1c(OC)c(F)cc2nc(N)sc12]

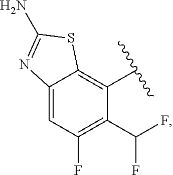 [SMILES: *c1c(C(F)F)c(F)cc2nc(N)sc12]

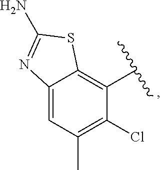 [SMILES: *c1c(Cl)c(C)cc2nc(N)sc12]

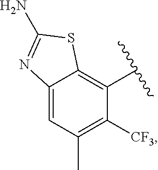 [SMILES: *c1c(C(F)(F)F)c(C)cc2nc(N)sc12]

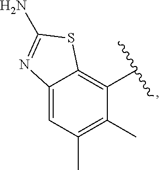 [SMILES: *c1c(C)c(C)cc2nc(N)sc12]

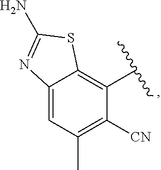 [SMILES: *c1c(C#N)c(C)cc2nc(N)sc12]

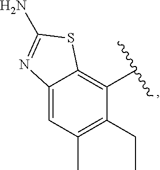 [SMILES: *c1c(CC)c(C)cc2nc(N)sc12]

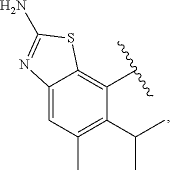 [SMILES: *c1c(C(C)C)c(C)cc2nc(N)sc12]

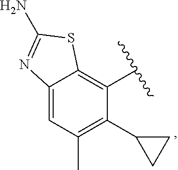 [SMILES: *c1c(C2CC2)c(C)cc2nc(N)sc12]

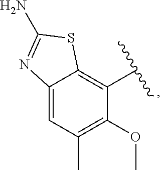 [SMILES: *c1c(OC)c(C)cc2nc(N)sc12]

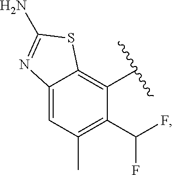 [SMILES: *c1c(C(F)F)c(C)cc2nc(N)sc12]

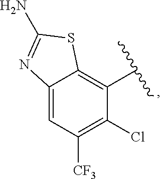 [SMILES: *c1c(Cl)c(C(F)(F)F)cc2nc(N)sc12]

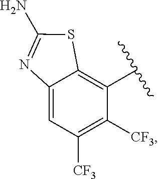 [SMILES: *c1c(C(F)(F)F)c(C(F)(F)F)cc2nc(N)sc12]

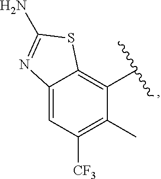 [SMILES: *c1c(C)c(C(F)(F)F)cc2nc(N)sc12]

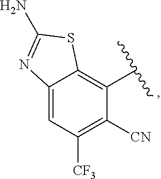 [SMILES: *c1c(C#N)c(C(F)(F)F)cc2nc(N)sc12]

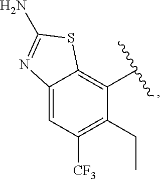 [SMILES: *c1c(CC)c(C(F)(F)F)cc2nc(N)sc12]

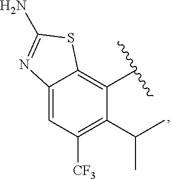 [SMILES: *c1c(C(C)C)c(C(F)(F)F)cc2nc(N)sc12]

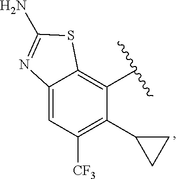 [SMILES: *c1c(C2CC2)c(C(F)(F)F)cc2nc(N)sc12]

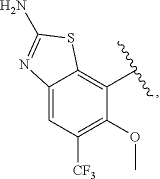 [SMILES: *c1c(OC)c(C(F)(F)F)cc2nc(N)sc12]

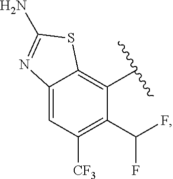 [SMILES: *c1c(C(F)F)c(C(F)(F)F)cc2nc(N)sc12]

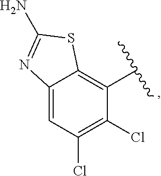 [SMILES: *c1c(Cl)c(Cl)cc2nc(N)sc12]

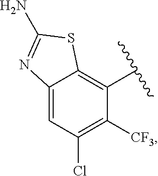 [SMILES: *c1c(C(F)(F)F)c(Cl)cc2nc(N)sc12]

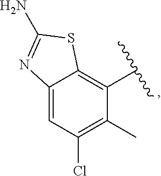 [SMILES: *c1c(C)c(Cl)cc2nc(N)sc12]

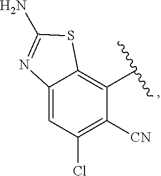 [SMILES: *c1c(C#N)c(Cl)cc2nc(N)sc12]

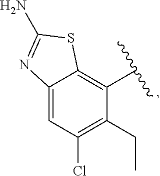 [SMILES: *c1c(CC)c(Cl)cc2nc(N)sc12]

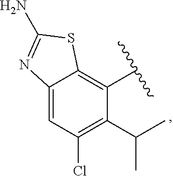 [SMILES: *c1c(C(C)C)c(Cl)cc2nc(N)sc12]

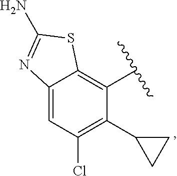 [SMILES: *c1c(C2CC2)c(Cl)cc2nc(N)sc12]

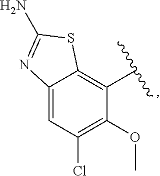 [SMILES: *c1c(OC)c(Cl)cc2nc(N)sc12]

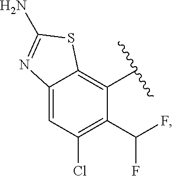 [SMILES: *c1c(C(F)F)c(Cl)cc2nc(N)sc12]

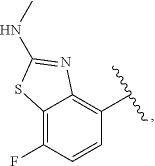 [SMILES: *c1ccc(F)c2sc(NC)nc12]

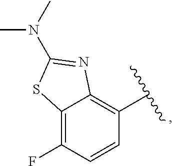 [SMILES: *c1ccc(F)c2sc(N(C)C)nc12]

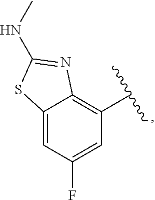 [SMILES: *c1cc(F)cc2sc(NC)nc12]

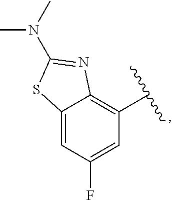 [SMILES: *c1cc(F)cc2sc(N(C)C)nc12]

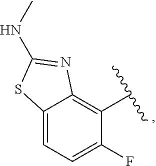 [SMILES: *c1c(F)ccc2sc(NC)nc12]

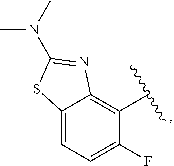 [SMILES: *c1c(F)ccc2sc(N(C)C)nc12]

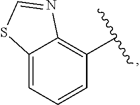 [SMILES: *c1cccc2scnc12]

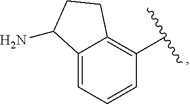 [SMILES: *c1cccc2c1CCC2N]

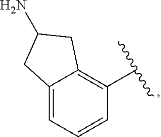 [SMILES: *c1cccc2c1CC(N)C2]

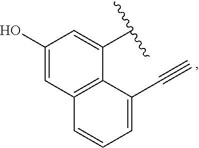 [SMILES: *c1cc(O)cc2cccc(C#C)c12]

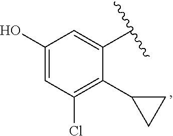 [SMILES: *c1cc(O)cc(Cl)c1C1CC1]

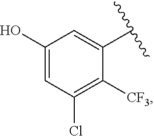 [SMILES: *c1cc(O)cc(Cl)c1C(F)(F)F]

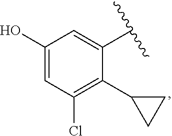 [SMILES: *c1cc(O)cc(Cl)c1C1CC1]

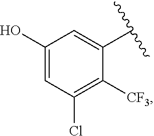 [SMILES: *c1cc(O)cc(Cl)c1C(F)(F)F]

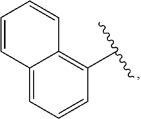 [SMILES: *c1cccc2ccccc12]

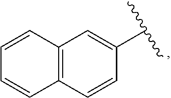 [SMILES: *c1ccc2ccccc2c1]

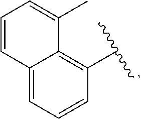 [SMILES: *c1cccc2cccc(C)c12]

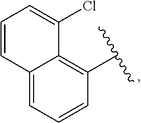 [SMILES: *c1cccc2cccc(Cl)c12]

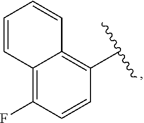 [SMILES: *c1ccc(F)c2ccccc12]

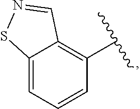 [SMILES: *c1cccc2sncc12]

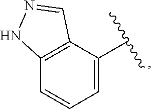 [SMILES: *c1cccc2[nH]ncc12]

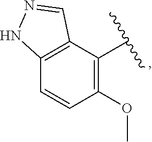 [SMILES: *c1c(OC)ccc2[nH]ncc12]

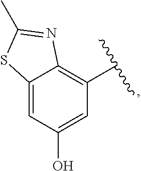 [SMILES: *c1cc(O)cc2sc(C)nc12]

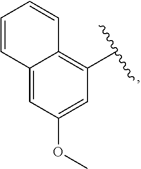 [SMILES: *c1cc(OC)cc2ccccc12]

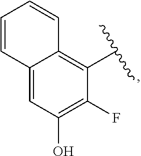 [SMILES: *c1c(F)c(O)cc2ccccc12]

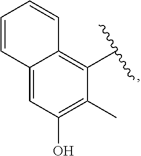 [SMILES: *c1c(C)c(O)cc2ccccc12]

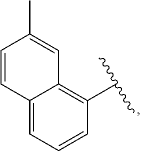 [SMILES: *c1cccc2ccc(C)cc12]

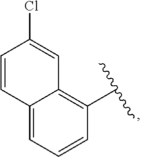 [SMILES: *c1cccc2ccc(Cl)cc12]

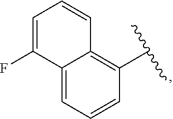 [SMILES: *c1cccc2c(F)cccc12]

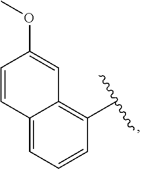 [SMILES: *c1cccc2ccc(OC)cc12]

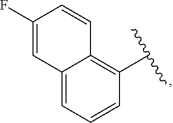 [SMILES: *c1cccc2cc(F)ccc12]

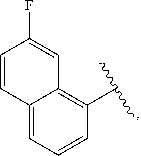 [SMILES: *c1cccc2ccc(F)cc12]

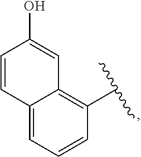 [SMILES: *c1cccc2ccc(O)cc12]

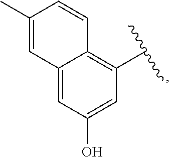 [SMILES: *c1cc(O)cc2cc(C)ccc12]

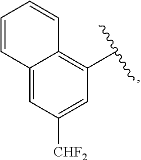 [SMILES: *c1cc(C(F)F)cc2ccccc12]

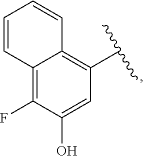 [SMILES: *c1cc(O)c(F)c2ccccc12]

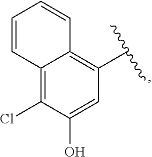 [SMILES: *c1cc(O)c(Cl)c2ccccc12]

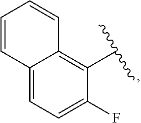 [SMILES: *c1c(F)ccc2ccccc12]

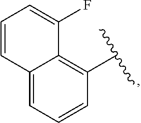 [SMILES: *c1cccc2cccc(F)c12]

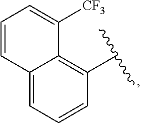 [SMILES: *c1cccc2cccc(C(F)(F)F)c12]

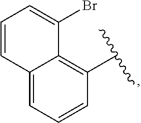 [SMILES: *c1cccc2cccc(Br)c12]

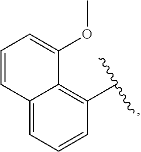 [SMILES: *c1cccc2cccc(OC)c12]

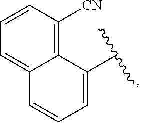 [SMILES: *c1cccc2cccc(C#N)c12]

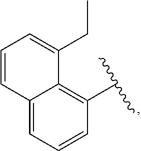 [SMILES: *c1cccc2cccc(CC)c12]

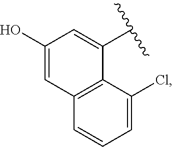 [SMILES: *c1cc(O)cc2cccc(Cl)c12]

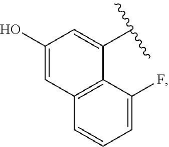 [SMILES: *c1cc(O)cc2cccc(F)c12]

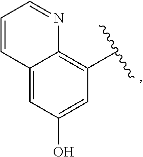 [SMILES: *c1cc(O)cc2cccnc12]

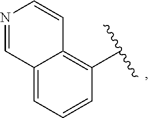 [SMILES: *c1cccc2cnccc12]

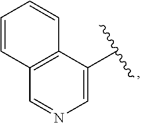 [SMILES: *c1cncc2ccccc12]

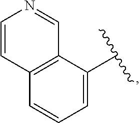 [SMILES: *c1cccc2ccncc12]

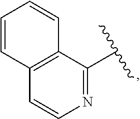 [SMILES: *c1nccc2ccccc12]

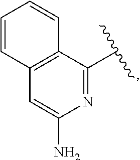 [SMILES: *c1nc(N)cc2ccccc12]

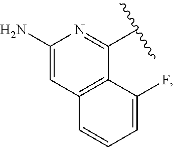 [SMILES: *c1nc(N)cc2cccc(F)c12]

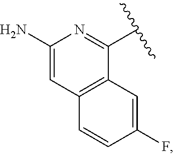 [SMILES: *c1nc(N)cc2ccc(F)cc12]

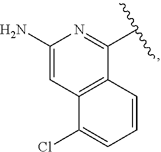 [SMILES: *c1nc(N)cc2c(Cl)cccc12]

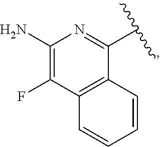 [SMILES: *c1nc(N)c(F)c2ccccc12]

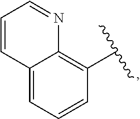 [SMILES: *c1cccc2cccnc12]

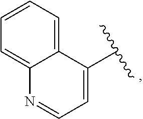 [SMILES: *c1ccnc2ccccc12]

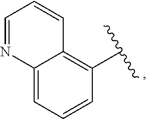 [SMILES: *c1cccc2ncccc12]

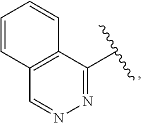 [SMILES: *c1nncc2ccccc12]

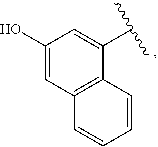 [SMILES: *c1cc(O)cc2ccccc12]

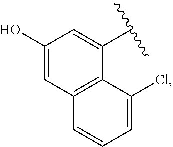 [SMILES: *c1cc(O)cc2cccc(Cl)c12]

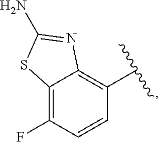 [SMILES: *c1ccc(F)c2sc(N)nc12]

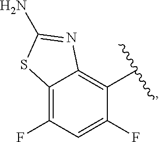 [SMILES: *c1c(F)cc(F)c2sc(N)nc12]

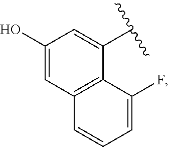 [SMILES: *c1cc(O)cc2cccc(F)c12]

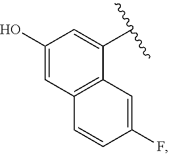 [SMILES: *c1cc(O)cc2ccc(F)cc12]

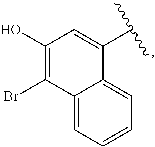 [SMILES: *c1cc(O)c(Br)c2ccccc12]

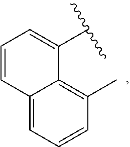 [SMILES: *c1cccc2cccc(C)c12]

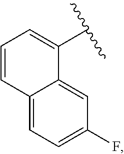 [SMILES: *c1cccc2ccc(F)cc12]

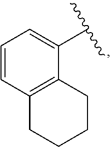 [SMILES: *c1cccc2c1CCCC2]

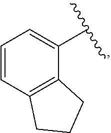 [SMILES: *c1cccc2c1CCC2]

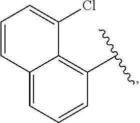 [SMILES: *c1cccc2cccc(Cl)c12]

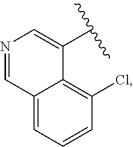 [SMILES: *c1cncc2cccc(Cl)c12]

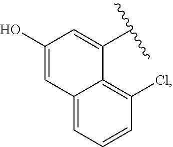 [SMILES: *c1cc(O)cc2cccc(Cl)c12]

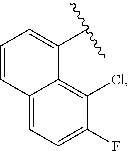 [SMILES: *c1cccc2ccc(F)c(Cl)c12]

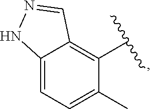 [SMILES: *c1c(C)ccc2[nH]ncc12]

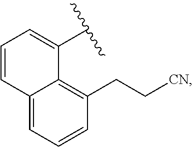 [SMILES: *c1cccc2cccc(CCC#N)c12]

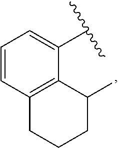

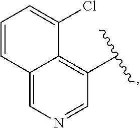 [SMILES: *c1cncc2cccc(Cl)c12]

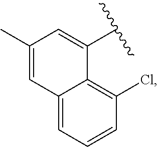 [SMILES: *c1cc(C)cc2cccc(Cl)c12]

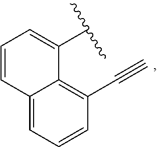 [SMILES: *c1cccc2cccc(C#C)c12]

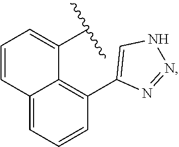 [SMILES: *c1cccc2cccc(-c3c[nH]nn3)c12]

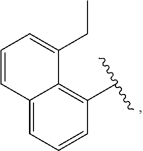 [SMILES: *c1cccc2cccc(CC)c12]

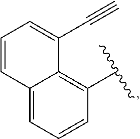 [SMILES: *c1cccc2cccc(C#C)c12]

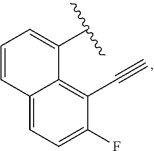 [SMILES: *c1cccc2ccc(F)c(C#C)c12]

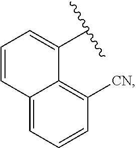 [SMILES: *c1cccc2cccc(C#N)c12]

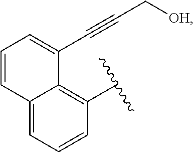 [SMILES: *c1cccc2cccc(C#CCO)c12]

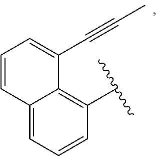 [SMILES: *c1cccc2cccc(C#CC)c12]

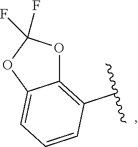 [SMILES: *c1cccc2c1OC(F)(F)O2]

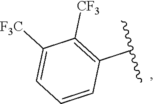 [SMILES: *c1cccc(C(F)(F)F)c1C(F)(F)F]

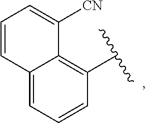 [SMILES: *c1cccc2cccc(C#N)c12]

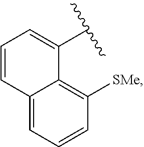 [SMILES: *c1cccc2cccc(SC)c12]

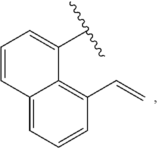 [SMILES: *c1cccc2cccc(C=C)c12]

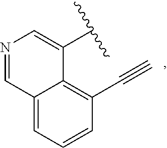 [SMILES: *c1cncc2cccc(C#C)c12]

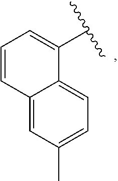 [SMILES: *c1cccc2cc(C)ccc12]

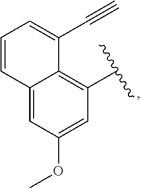 [SMILES: *c1cc(OC)cc2cccc(C#C)c12]

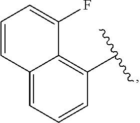 [SMILES: *c1cccc2cccc(F)c12]

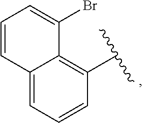 [SMILES: *c1cccc2cccc(Br)c12]

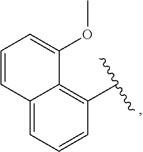 [SMILES: *c1cccc2cccc(OC)c12]

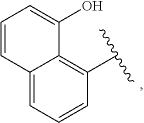 [SMILES: *c1cccc2cccc(O)c12]

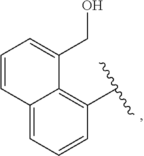 [SMILES: *c1cccc2cccc(CO)c12]

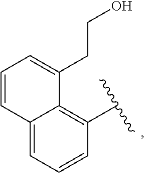 [SMILES: *c1cccc2cccc(CCO)c12]

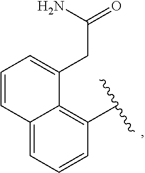 [SMILES: *c1cccc2cccc(CC(N)=O)c12]

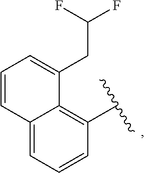 [SMILES: *c1cccc2cccc(CC(F)F)c12]

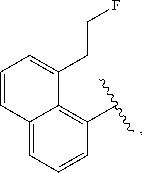 [SMILES: *c1cccc2cccc(CCF)c12]

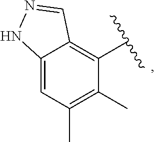 [SMILES: *c1c(C)c(C)cc2[nH]ncc12]

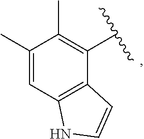 [SMILES: *c1c(C)c(C)cc2[nH]ccc12]

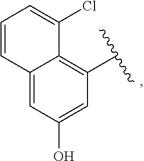 [SMILES: *c1cc(O)cc2cccc(Cl)c12]

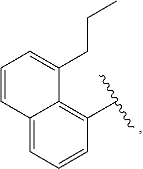 [SMILES: *c1cccc2cccc(CCC)c12]

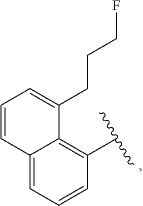 [SMILES: *c1cccc2cccc(CCCF)c12]

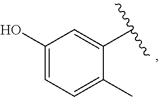 [SMILES: *c1cc(O)ccc1C]

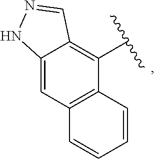 [SMILES: *c1c2ccccc2cc2[nH]ncc12]

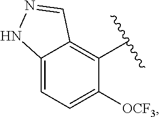 [SMILES: *c1c(OC(F)(F)F)ccc2[nH]ncc12]

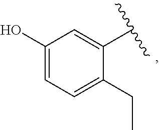 [SMILES: *c1cc(O)ccc1CC]

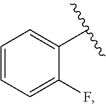 [SMILES: *c1ccccc1F]

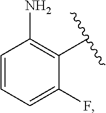 [SMILES: *c1c(N)cccc1F]

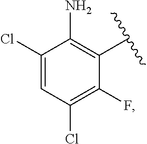 [SMILES: *c1c(N)c(Cl)cc(Cl)c1F]

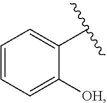 [SMILES: *c1ccccc1O]

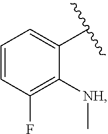 [SMILES: *c1cccc(F)c1NC]

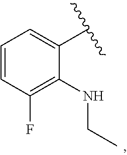 [SMILES: *c1cccc(F)c1NCC]

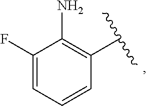 [SMILES: *c1cccc(F)c1N]

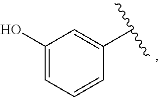 [SMILES: *c1cccc(O)c1]

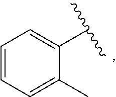 [SMILES: *c1ccccc1C]

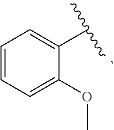 [SMILES: *c1ccccc1OC]

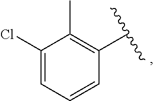 [SMILES: *c1cccc(Cl)c1C]

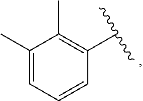 [SMILES: *c1cccc(C)c1C]

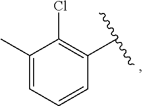 [SMILES: *c1cccc(C)c1Cl]

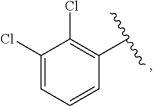 [SMILES: *c1cccc(Cl)c1Cl]

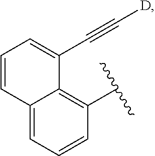

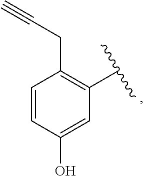 [SMILES: *c1cc(O)ccc1CC#C]

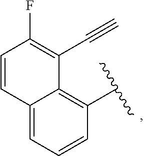 [SMILES: *c1cccc2ccc(F)c(C#C)c12]

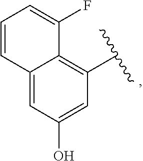 [SMILES: *c1cc(O)cc2cccc(F)c12]

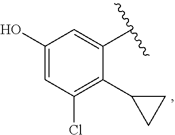 [SMILES: *c1cc(O)cc(Cl)c1C1CC1]

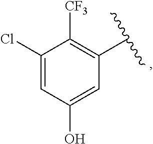 [SMILES: *c1cc(O)cc(Cl)c1C(F)(F)F]

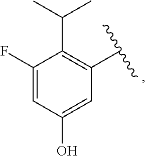 [SMILES: *c1cc(O)cc(F)c1C(C)C]

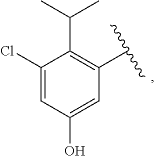 [SMILES: *c1cc(O)cc(Cl)c1C(C)C]

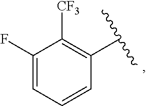 [SMILES: *c1cccc(F)c1C(F)(F)F]

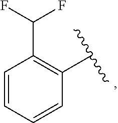 [SMILES: *c1ccccc1C(F)F]

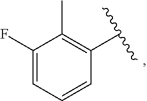 [SMILES: *c1cccc(F)c1C]

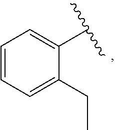 [SMILES: *c1ccccc1CC]

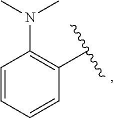 [SMILES: *c1ccccc1N(C)C]

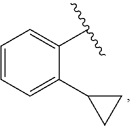 [SMILES: *c1ccccc1C1CC1]

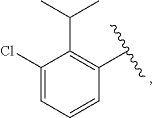 [SMILES: *c1cccc(Cl)c1C(C)C]

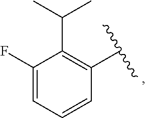 [SMILES: *c1cccc(F)c1C(C)C]

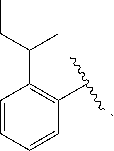 [SMILES: *c1ccccc1C(C)CC]

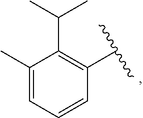 [SMILES: *c1cccc(C)c1C(C)C]

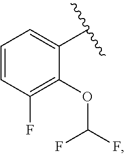 [SMILES: *c1cccc(F)c1OC(F)F]

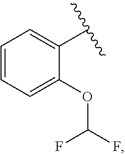 [SMILES: *c1ccccc1OC(F)F]

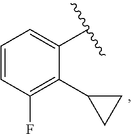 [SMILES: *c1cccc(F)c1C1CC1]

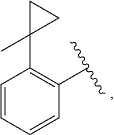 [SMILES: *c1ccccc1C1(C)CC1]

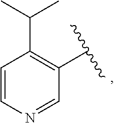 [SMILES: *c1cnccc1C(C)C]

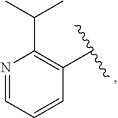 [SMILES: *c1cccnc1C(C)C]

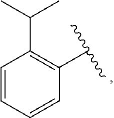 [SMILES: *c1ccccc1C(C)C]

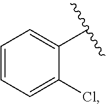 [SMILES: *c1ccccc1Cl]

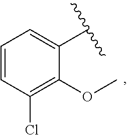 [SMILES: *c1cccc(Cl)c1OC]

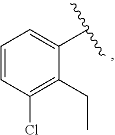 [SMILES: *c1cccc(Cl)c1CC]

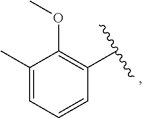 [SMILES: *c1cccc(C)c1OC]

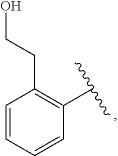 [SMILES: *c1ccccc1CCO]

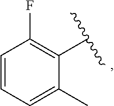 [SMILES: *c1c(C)cccc1F]

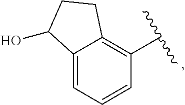 [SMILES: *c1cccc2c1CCC2O]

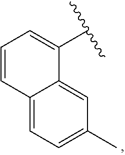 [SMILES: *c1cccc2ccc(C)cc12]

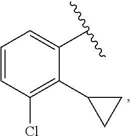 [SMILES: *c1cccc(Cl)c1C1CC1]

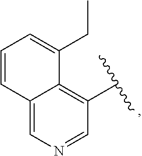 [SMILES: *c1cncc2cccc(CC)c12]

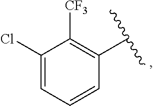 [SMILES: *c1cccc(Cl)c1C(F)(F)F]

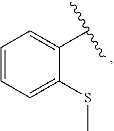 [SMILES: *c1ccccc1SC]

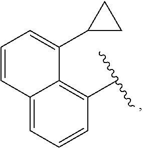 [SMILES: *c1cccc2cccc(C3CC3)c12]

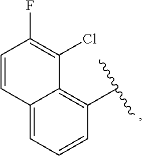 [SMILES: *c1cccc2ccc(F)c(Cl)c12]

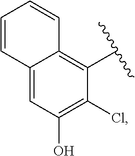 [SMILES: *c1c(Cl)c(O)cc2ccccc12]

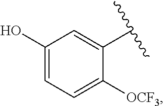 [SMILES: *c1cc(O)ccc1OC(F)(F)F]

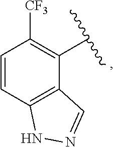 [SMILES: *c1c(C(F)(F)F)ccc2[nH]ncc12]

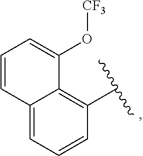 [SMILES: *c1cccc2cccc(OC(F)(F)F)c12]

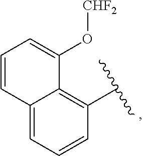 [SMILES: *c1cccc2cccc(OC(F)F)c12]

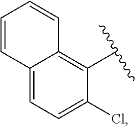 [SMILES: *c1c(Cl)ccc2ccccc12]

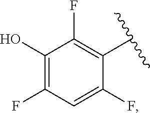 [SMILES: *c1c(F)cc(F)c(O)c1F]

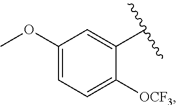 [SMILES: *c1cc(OC)ccc1OC(F)(F)F]

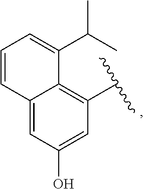 [SMILES: *c1cc(O)cc2cccc(C(C)C)c12]

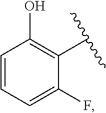 [SMILES: *c1c(O)cccc1F]

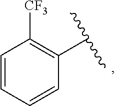 [SMILES: *c1ccccc1C(F)(F)F]

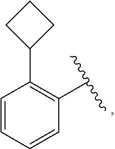 [SMILES: *c1ccccc1C1CCC1]

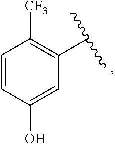 [SMILES: *c1cc(O)ccc1C(F)(F)F]

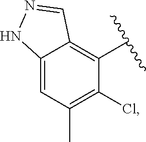 [SMILES: *c1c(Cl)c(C)cc2[nH]ncc12]

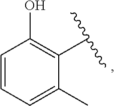 [SMILES: *c1c(C)cccc1O]

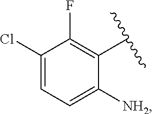 [SMILES: *c1c(N)ccc(Cl)c1F]

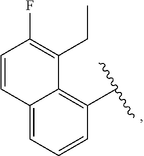 [SMILES: *c1cccc2ccc(F)c(CC)c12]

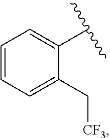 [SMILES: *c1ccccc1CC(F)(F)F]

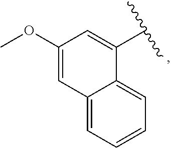 [SMILES: *c1cc(OC)cc2ccccc12]

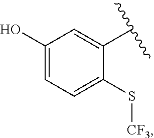 [SMILES: *c1cc(O)ccc1SC(F)(F)F]

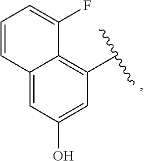 [SMILES: *c1cc(O)cc2cccc(F)c12]

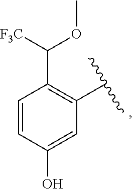 [SMILES: *c1cc(O)ccc1C(OC)C(F)(F)F]

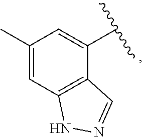 [SMILES: *c1cc(C)cc2[nH]ncc12]

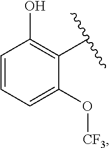 [SMILES: *c1c(O)cccc1OC(F)(F)F]

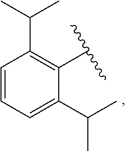 [SMILES: *c1c(C(C)C)cccc1C(C)C]

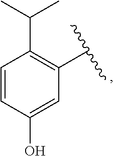 [SMILES: *c1cc(O)ccc1C(C)C]

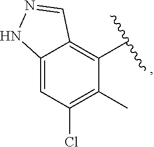 [SMILES: *c1c(C)c(Cl)cc2[nH]ncc12]

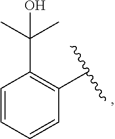 [SMILES: *c1ccccc1C(C)(C)O]

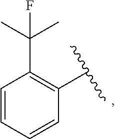 [SMILES: *c1ccccc1C(C)(C)F]

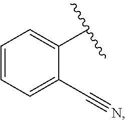 [SMILES: *c1ccccc1C#N]

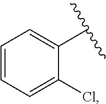 [SMILES: *c1ccccc1Cl]

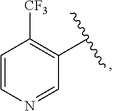 [SMILES: *c1cnccc1C(F)(F)F]

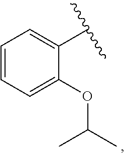 [SMILES: *c1ccccc1OC(C)C]

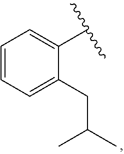 [SMILES: *c1ccccc1CC(C)C]

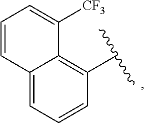 [SMILES: *c1cccc2cccc(C(F)(F)F)c12]

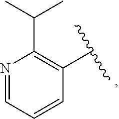 [SMILES: *c1cccnc1C(C)C]

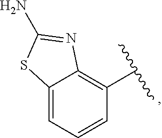 [SMILES: *c1cccc2sc(N)nc12]

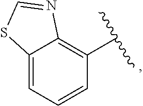 [SMILES: *c1cccc2scnc12]

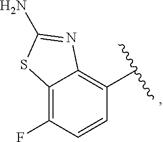 [SMILES: *c1ccc(F)c2sc(N)nc12]

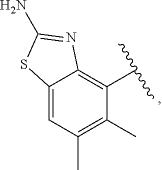 [SMILES: *c1c(C)c(C)cc2sc(N)nc12]

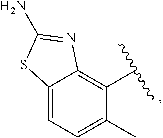 [SMILES: *c1c(C)ccc2sc(N)nc12]

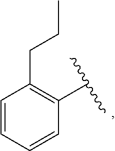 [SMILES: *c1ccccc1CCC]

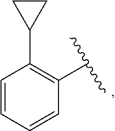 [SMILES: *c1ccccc1C1CC1]

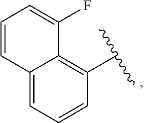 [SMILES: *c1cccc2cccc(F)c12]

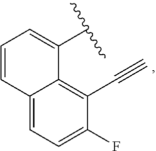 [SMILES: *c1cccc2ccc(F)c(C#C)c12]

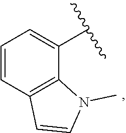 [SMILES: *c1cccc2ccn(C)c12]

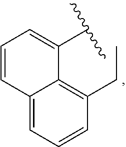 [SMILES: *c1cccc2cccc(CC)c12]

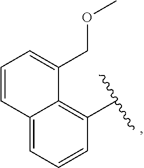 [SMILES: *c1cccc2cccc(COC)c12]

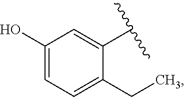 [SMILES: *c1cc(O)ccc1CC]

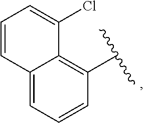 [SMILES: *c1cccc2cccc(Cl)c12]

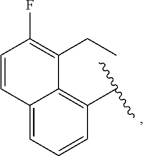 [SMILES: *c1cccc2ccc(F)c(CC)c12]

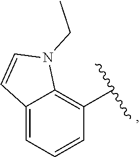 [SMILES: *c1cccc2ccn(CC)c12]

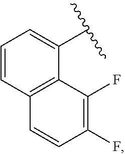 [SMILES: *c1cccc2ccc(F)c(F)c12]

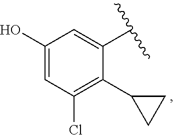 [SMILES: *c1cc(O)cc(Cl)c1C1CC1]

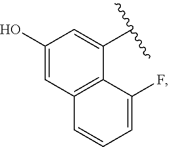 [SMILES: *c1cc(O)cc2cccc(F)c12]

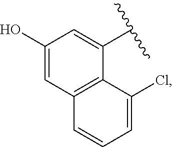 [SMILES: *c1cc(O)cc2cccc(Cl)c12]

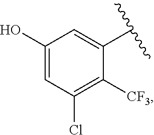 [SMILES: *c1cc(O)cc(Cl)c1C(F)(F)F]

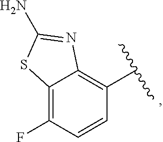 [SMILES: *c1ccc(F)c2sc(N)nc12]

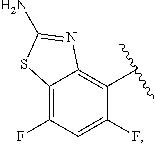 [SMILES: *c1c(F)cc(F)c2sc(N)nc12]

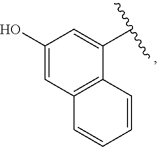 [SMILES: *c1cc(O)cc2ccccc12]

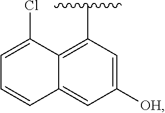 [SMILES: *c1cc(O)cc2cccc(Cl)c12]

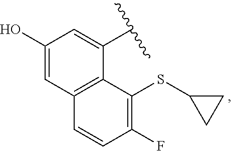 [SMILES: *c1cc(O)cc2ccc(F)c(SC3CC3)c12]

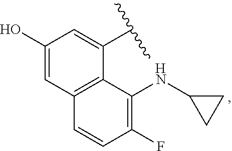

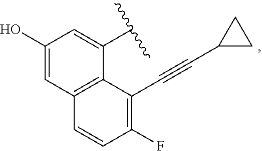 [SMILES: *c1cc(O)cc2ccc(F)c(C#CC3CC3)c12]

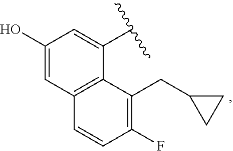 [SMILES: *c1cc(O)cc2ccc(F)c(CC3CC3)c12]

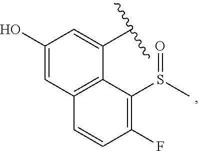 [SMILES: *c1cc(O)cc2ccc(F)c(S(C)=O)c12]

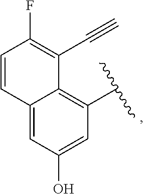 [SMILES: *c1cc(O)cc2ccc(F)c(C#C)c12]

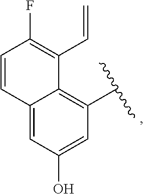 [SMILES: *c1cc(O)cc2ccc(F)c(C=C)c12]

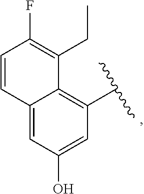 [SMILES: *c1cc(O)cc2ccc(F)c(CC)c12]

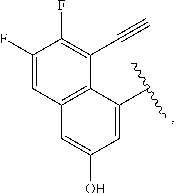 [SMILES: *c1cc(O)cc2cc(F)c(F)c(C#C)c12]

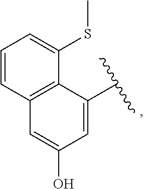 [SMILES: *c1cc(O)cc2cccc(SC)c12]

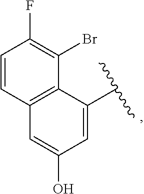 [SMILES: *c1cc(O)cc2ccc(F)c(Br)c12]

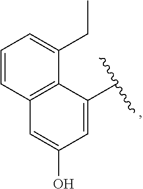 [SMILES: *c1cc(O)cc2cccc(CC)c12]

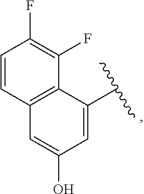 [SMILES: *c1cc(O)cc2ccc(F)c(F)c12]

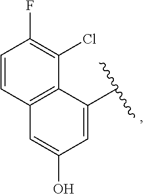 [SMILES: *c1cc(O)cc2ccc(F)c(Cl)c12]

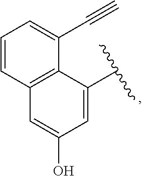 [SMILES: *c1cc(O)cc2cccc(C#C)c12]

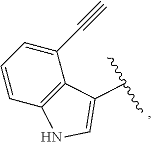 [SMILES: *c1c[nH]c2cccc(C#C)c12]

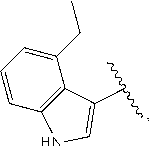 [SMILES: *c1c[nH]c2cccc(CC)c12]

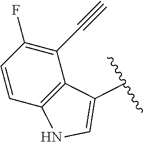 [SMILES: *c1c[nH]c2ccc(F)c(C#C)c12]

or

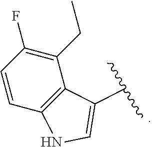 [SMILES: *c1c[nH]c2ccc(F)c(CC)c12]

[24]. The compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of any one of [1] to [23], wherein, R₄ is selected from any moiety in the Table 10:

TABLE 10

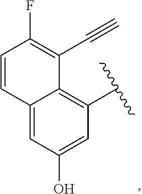 [SMILES: *c1cc(O)cc2ccc(F)c(C#C)c12]

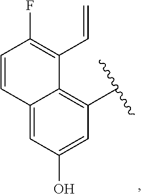 [SMILES: *c1cc(O)cc2ccc(F)c(C=C)c12]

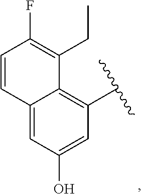 [SMILES: *c1cc(O)cc2ccc(F)c(CC)c12]

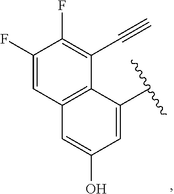 [SMILES: *c1cc(O)cc2cc(F)c(F)c(C#C)c12]

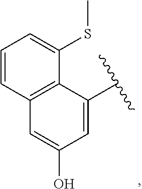 [SMILES: *c1cc(O)cc2cccc(SC)c12]

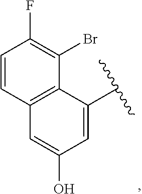 [SMILES: *c1cc(O)cc2ccc(F)c(Br)c12]

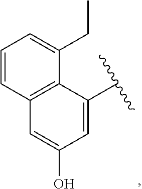 [SMILES: *c1cc(O)cc2cccc(CC)c12]

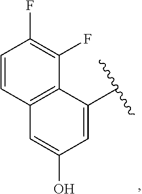 [SMILES: *c1cc(O)cc2ccc(F)c(F)c12]

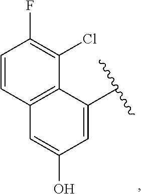 [SMILES: *c1cc(O)cc2ccc(F)c(Cl)c12]

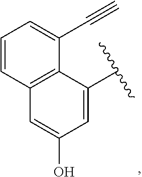 [SMILES: *c1cc(O)cc2cccc(C#C)c12]

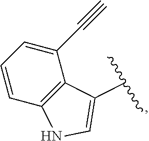 [SMILES: *c1c[nH]c2cccc(C#C)c12]

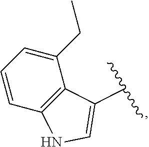 [SMILES: *c1c[nH]c2cccc(CC)c12]

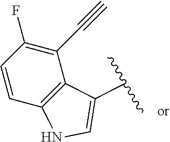 [SMILES: *c1c[nH]c2ccc(F)c(C#C)c12]

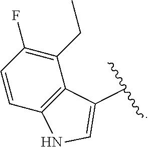 [SMILES: *c1c[nH]c2ccc(F)c(CC)c12]

[25]. The compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of any one of [1] to [24], wherein, R₅₁ is selected from hydrogen, deuterium, —F, —Cl, —Br, —C₁₋₃alkyl, haloC₁₋₃alkyl, haloC₁₋₃alkoxy, —CN, —NHC₁₋₃alkyl, —N(C₁₋₃alkyl)₂, —OC₁₋₃alkyl, —O-(3-6 membered cycloalkyl), —SC₁₋₃alkyl, —S(haloC₁₋₃alkyl) or 3-6 membered cycloalkyl; wherein, said —C₁₋₃alkyl or 3-6 membered cycloalkyl is optionally substituted with 1, 2 or 3 substituents selected from halogen, —C₁₋₃alkyl, haloC₁₋₃alkyl, haloC₁₋₃alkoxy, —CN, —NH₂, —NH(C₁₋₃alkyl), —N(C₁₋₃alkyl)₂, —OH, —OC₁₋₃alkyl, —SH, —SC₁₋₃alkyl or —S(haloC₁₋₃alkyl).

[26]. The compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of any one of [1] to [25], wherein, R₅₁ is selected from hydrogen, deuterium, —Cl, —CN, —CH₃, —CHF₂, —CH₂F, —CF₃, —CH₂OH, —CH₂CH₃, —OCH₃, —OCH₂CH₃, —SCH₃, —NHCH₃, —N(CH₃)₂, —OCF₃, —CN, —CH₂CN, —COOH, —CONH₂, —COOCH₃,

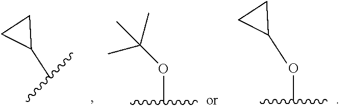

[27]. The compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of any one of [1] to [26], wherein, R₅₁ is selected from hydrogen.

[28]. The compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of any one of [1] to [27], wherein, the compound is selected from any one of the following formulas:

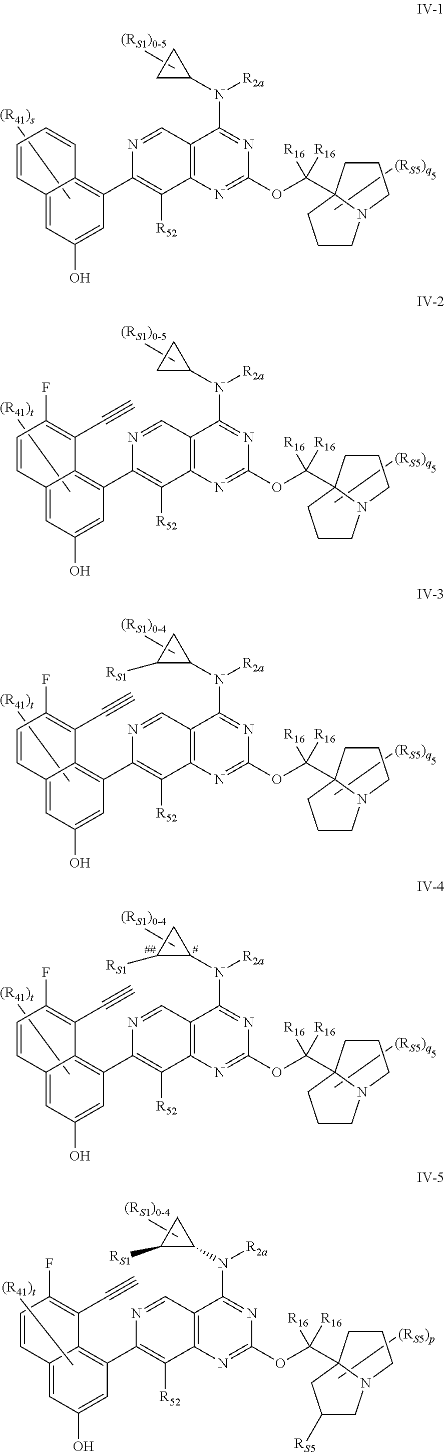

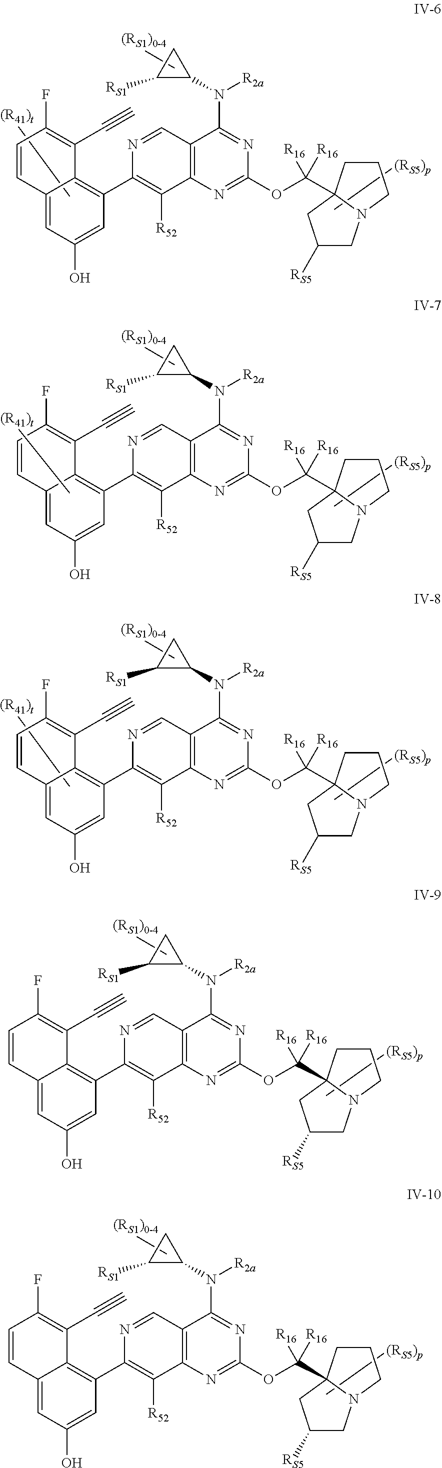

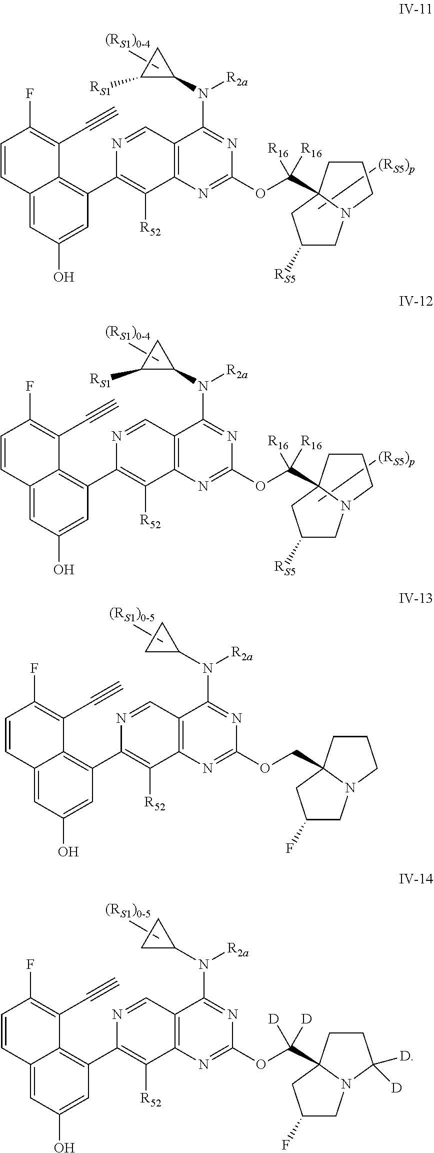

[29]. The compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of any one of [1] to [28], wherein, R₅₂ is selected from halogen.

[30]. The compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of any one of [1] to [29], wherein, R₅₂ is selected from —F.

[31]. The compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of any one of [1] to [30], wherein, the compound is selected from any one of the following formulas:

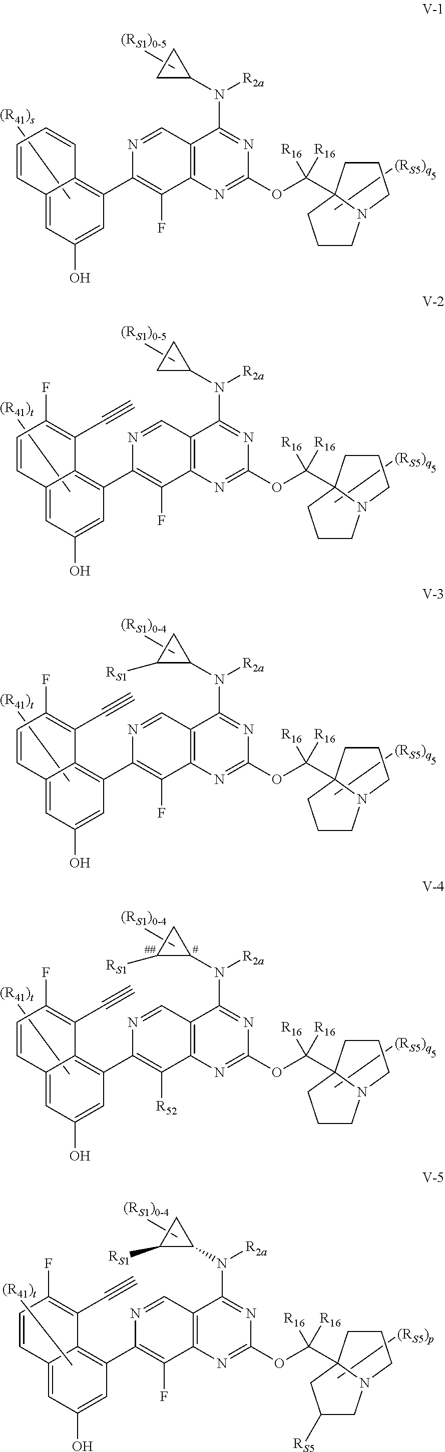

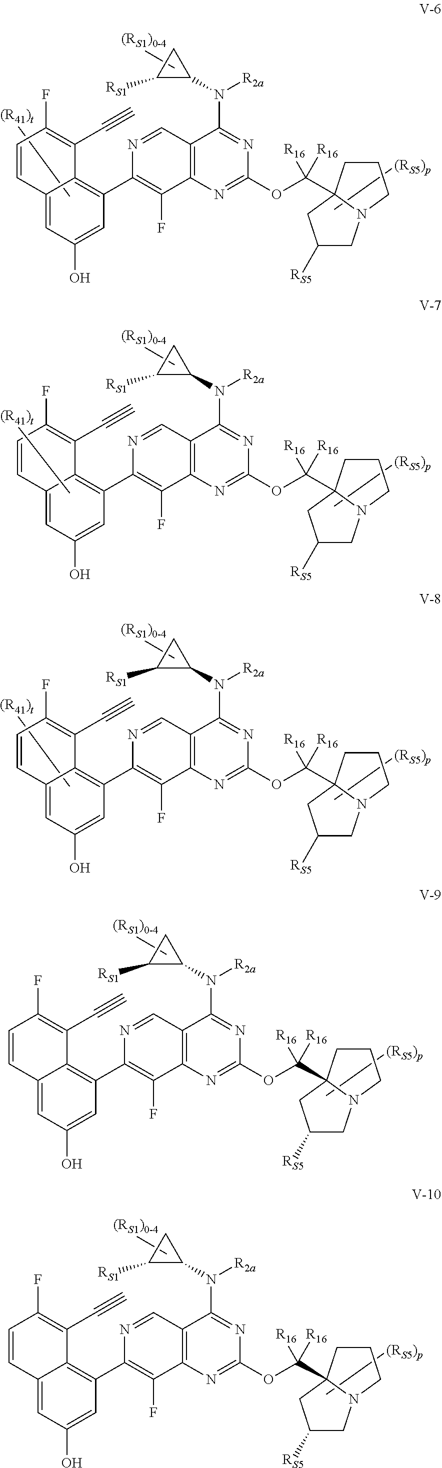

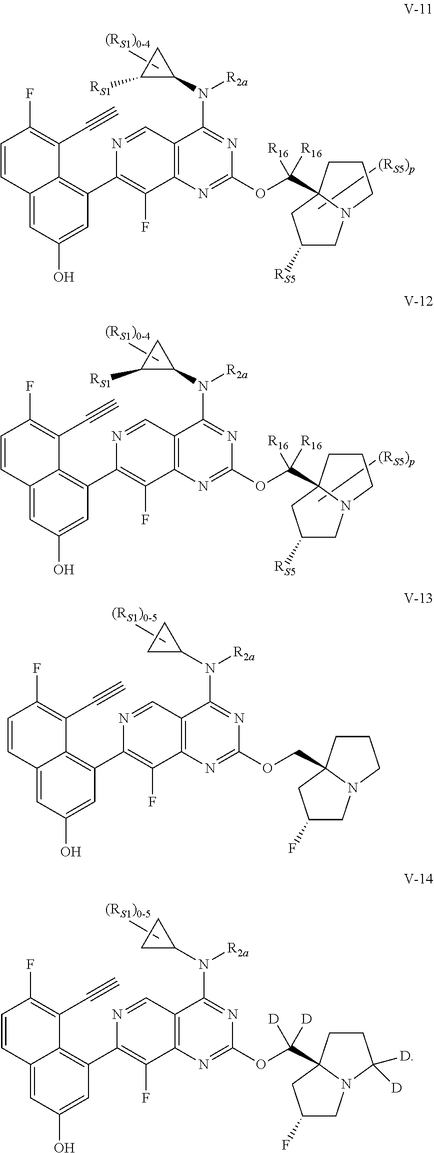

[32]. The compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of any one of [1] to [31], wherein, the prodrug is selected from any one of the following formulas:

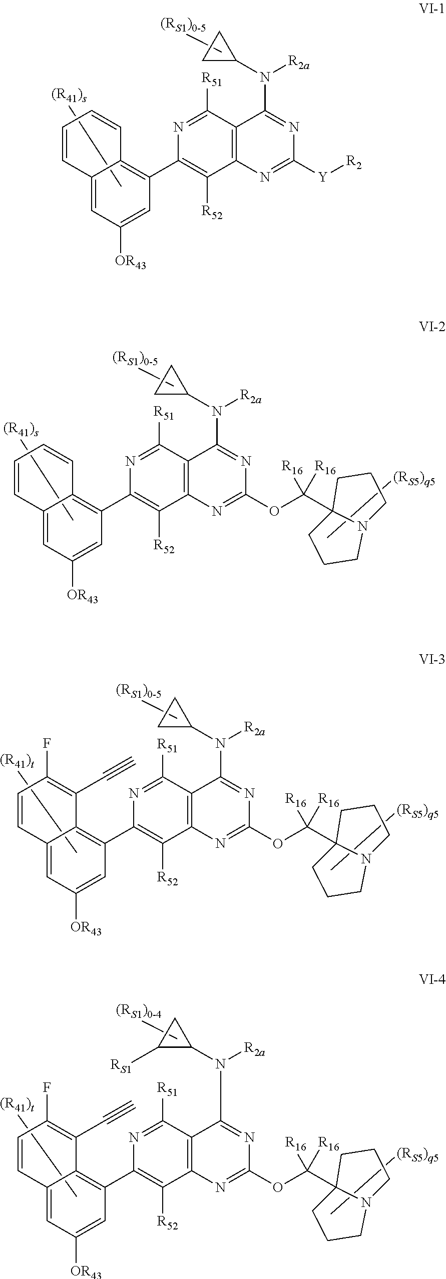

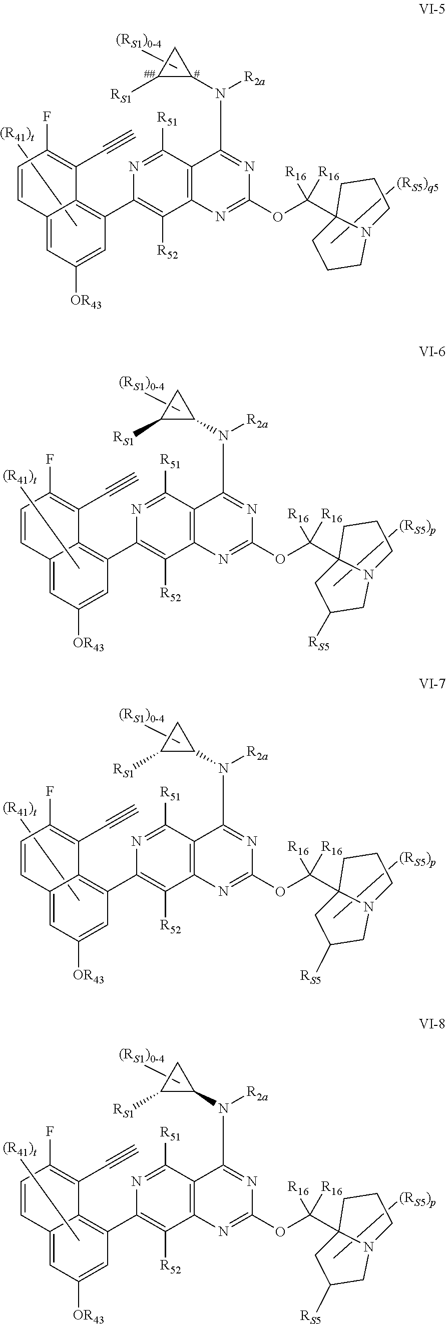

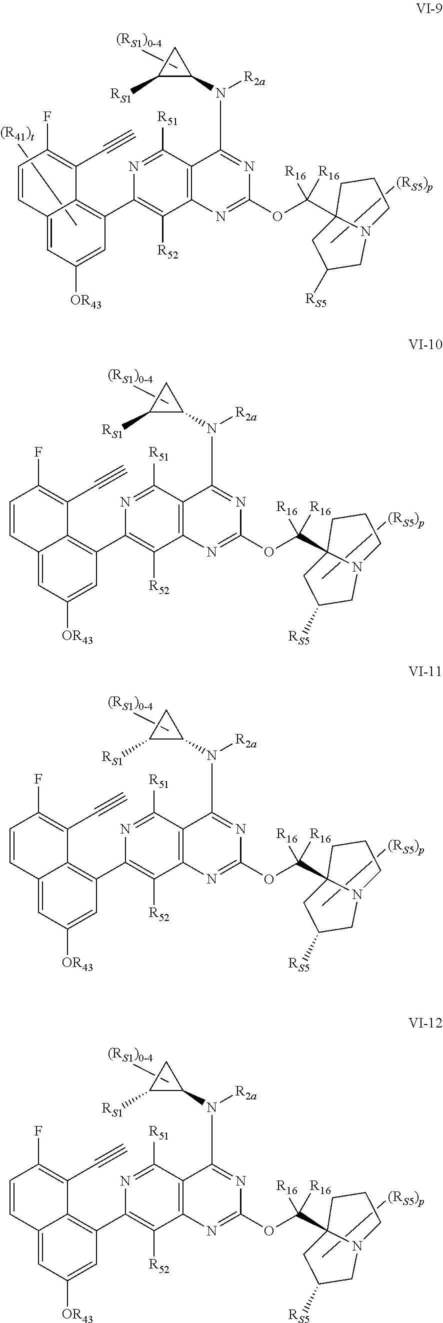

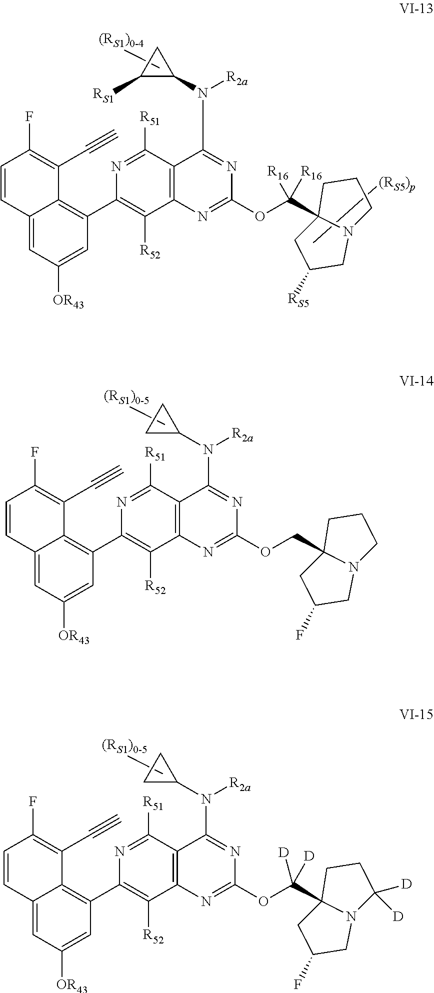

R₄₃ at each occurrence is independently selected from

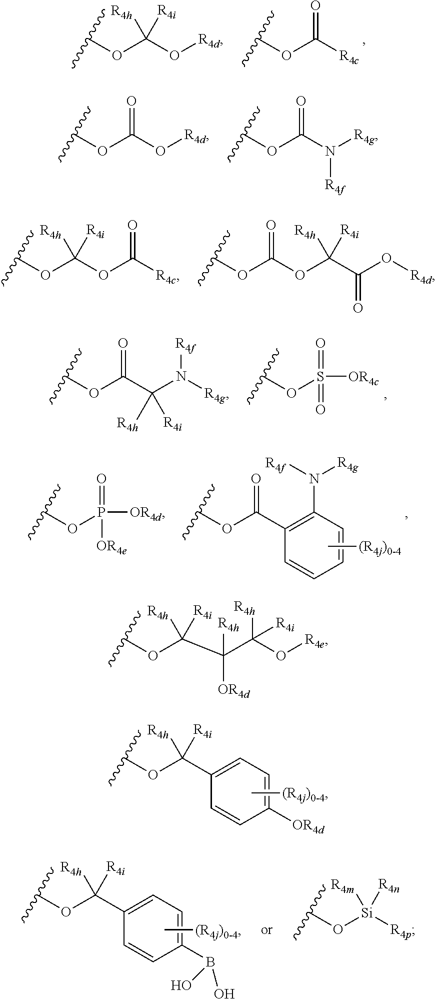

R_{4c} is selected from hydrogen, —C₁₋₃₀alkyl, —C₂₋₃₀alkenyl, —C₂₋₃₀alkynyl, —C₀₋₆alkylene-(3-20 membered carbocyclyl), —C₀₋₆alkylene-(3-20 membered heterocyclyl), —C₀₋₆alkylene-(6-10 membered aryl) or —C₀₋₆alkylene-(5-10 membered heteroaryl), each of which is independently substituted with one or more R₄ⱼ;
R_{4d} and R₄ₑ are each selected from hydrogen, —C₁₋₃₀alkyl, —C₂₋₃₀alkenyl, —C₂₋₃₀alkynyl, —C(═O)C₁₋₆alkyl, —C₀₋₆alkylene-(3-20 membered carbocyclyl), —C₀₋₆alkylene-(3-20 membered heterocyclyl), —C₀₋₆alkylene-(6-10 membered aryl) or —C₀₋₆alkylene-(5-10 membered heteroaryl), each of which is independently substituted with one or more 14;
R_{4f} and R_{4g} are each selected from hydrogen, —C₁₋₃₀alkyl, —C₂₋₃₀alkenyl, —C₂₋₃₀alkynyl, —C(═O)C₁₋₆alkyl, —C₀₋₆alkylene-(3-20 membered carbocyclyl), —C₀₋₆alkylene-(3-20 membered heterocyclyl), —C₀₋₆alkylene-(6-10 membered aryl) or —C₀₋₆alkylene-(5-10 membered heteroaryl), each of which is independently substituted with one or more 14;
R₄ₕ, R₄ᵢ, R₄ₘ, R₄ₙ and R₄ₚ are each selected from hydrogen, halogen, —C₁₋₆alkyl, haloC₁₋₆alkyl, haloC₁₋₆alkoxy, —C₂₋₆alkenyl, —C₂₋₆alkynyl, —CN, —NH₂, —NH(C₁₋₆alkyl), —N(C₁₋₆alkyl)₂, oxo, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl or 5-10 membered heteroaryl; wherein, said —C₁₋₆alkyl, —C₂₋₆alkenyl, —C₂₋₆alkynyl, 3-10 membered cycloalkyl, 3-10 membered heterocyclyl, 6-10 membered aryl or 5-10 membered heteroaryl is optionally substituted with one or more substituents selected from halogen, —C₁₋₆alkyl, haloC₁₋₆alkyl, haloC₁₋₆alkoxy, —C₂₋₆alkenyl, —C₂₋₆alkynyl, —CN, —NH₂, —NH(C₁₋₆alkyl), —N(C₁₋₆alkyl)₂, oxo, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl or 5-10 membered heteroaryl;
Optionally, R_{4f} and R_{4g} together with the atoms to which they are respectively attached form a 4-10 membered heterocyclyl ring, said 4-10 membered heterocyclyl ring optionally further contains 1 or 2 heteroatoms selected from N, O, S, S(═O) or S(═O)₂ and optionally substituted with one or more R₄ⱼ;
Optionally, R_{4f} and R₄ₕ together with the atoms to which they are respectively attached form a 4-10 membered heterocyclyl ring, said 4-10 membered heterocyclyl ring optionally further contains 1 or 2 heteroatoms selected from N, O, S, S(═O) or S(═O)₂ and optionally substituted with one or more R₄ⱼ;
R₄ⱼ at each occurrence is independently selected from halogen, —C₁₋₆alkyl, haloC₁₋₆alkyl, haloC₁₋₆alkoxy, —C₂₋₆alkenyl, —C₂₋₆alkynyl, —CN, oxo, —NO₂, —NH₂, —NH(C₁₋₆alkyl), —N(C₁₋₆alkyl)₂, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl, or 5-10 membered heteroaryl, wherein said —C₁₋₆alkyl, haloC₁₋₆alkyl, haloC₁₋₆alkoxy, —C₂₋₆alkenyl, —C₂₋₆alkynyl, 3-10 membered cycloalkyl, 3-10 membered heterocyclyl, 6-10 membered aryl or 5-10 membered heteroaryl is independently optionally substituted with 1, 2 or 3 substituents selected from halogen; —C₁₋₆alkyl; haloC₁₋₆alkyl; —CN; oxo; —OH; —NH₂; —NH(C₁₋₆alkyl); —N(C₁₋₆alkyl)₂; —OC₁₋₆alkyl; or —C₁₋₆alkyl substituted with 1, 2 or 3 substituents selected from halogen, haloC₁₋₆alkyl, —CN, —OH, —NH₂, —NH(C₁₋₆alkyl), —N(C₁₋₆alkyl)₂ or —OC₁₋₆alkyl;
Each of (heterocyclyl and heteroaryl) at each occurrence is independently contain 1, 2, 3 or 4 heteroatoms selected from N, O, S, S(═O) or S(═O)₂.

[33]. The compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of [32], wherein, —OR₄₃ is selected from any moiety in the Table 11:

TABLE 11

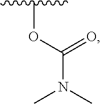 [SMILES: *OC(=O)N(C)C]

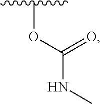 [SMILES: *OC(=O)NC]

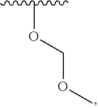 [SMILES: *OCOC]

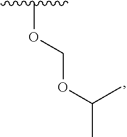 [SMILES: *OCOC(C)C]

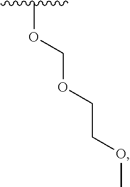 [SMILES: *OCOCCOC]

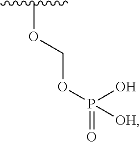 [SMILES: *OCOP(=O)(O)O]

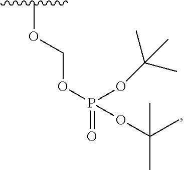 [SMILES: *OCOP(=O)(OC(C)(C)C)OC(C)(C)C]

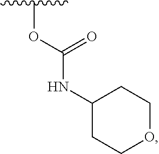 [SMILES: *OC(=O)NC1CCOCC1]

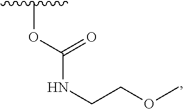 [SMILES: *OC(=O)NCCOC]

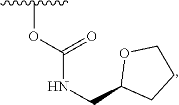 [SMILES: *OC(=O)NC[C@@H]1CCCO1]

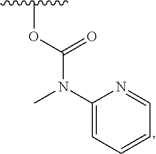 [SMILES: *OC(=O)N(C)c1ccccn1]

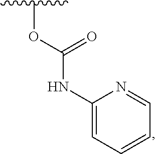 [SMILES: *OC(=O)Nc1ccccn1]

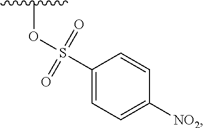 [SMILES: *OS(=O)(=O)c1ccc([N+](=O)[O-])cc1]

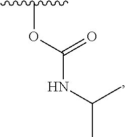 [SMILES: *OC(=O)NC(C)C]

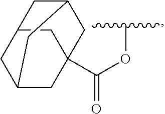 [SMILES: *OC(=O)C12CC3CC(CC(C3)C1)C2]

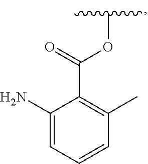 [SMILES: *OC(=O)c1c(C)cccc1N]

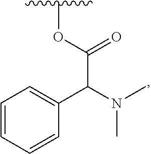 [SMILES: *OC(=O)C(c1ccccc1)N(C)C]

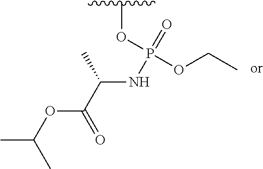 [SMILES: *OP(=O)(N[C@@H](C)C(=O)OC(C)C)OCC]

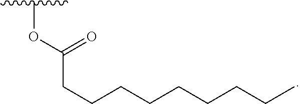 [SMILES: *OC(=O)CCCCCCCCC]

[34]. The compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of [32] or [33], wherein, the moiety of

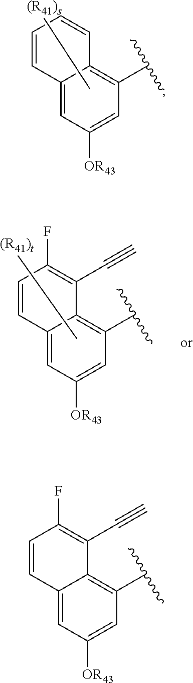

is selected from any moiety in the Table 12:

TABLE 12

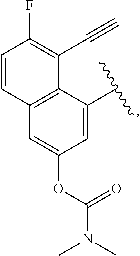 [SMILES: *c1cc(OC(=O)N(C)C)cc2ccc(F)c(C#C)c12]

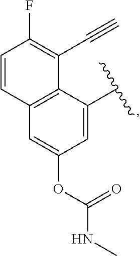 [SMILES: *c1cc(OC(=O)NC)cc2ccc(F)c(C#C)c12]

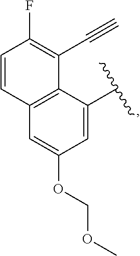 [SMILES: *c1cc(OCOC)cc2ccc(F)c(C#C)c12]

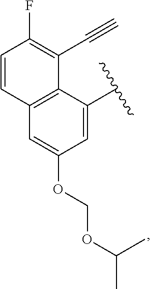 [SMILES: *c1cc(OCOC(C)C)cc2ccc(F)c(C#C)c12]

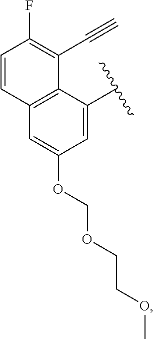 [SMILES: *c1cc(OCOCCOC)cc2ccc(F)c(C#C)c12]

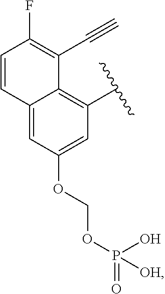 [SMILES: *c1cc(OCOP(=O)(O)O)cc2ccc(F)c(C#C)c12]

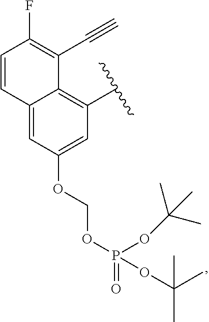 [SMILES: *c1cc(OCOP(=O)(OC(C)(C)C)OC(C)(C)C)cc2ccc(F)c(C#C)c12]

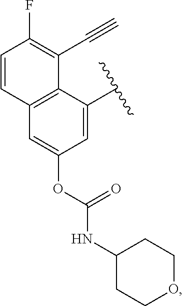 [SMILES: *c1cc(OC(=O)NC2CCOCC2)cc2ccc(F)c(C#C)c12]

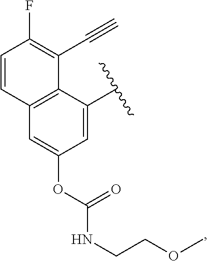 [SMILES: *c1cc(OC(=O)NCCOC)cc2ccc(F)c(C#C)c12]

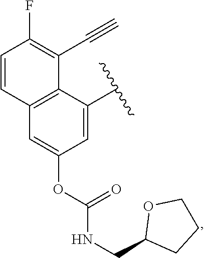 [SMILES: *c1cc(OC(=O)NC[C@@H]2CCCO2)cc2ccc(F)c(C#C)c12]

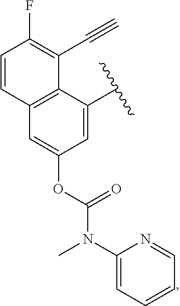 [SMILES: *c1cc(OC(=O)N(C)c2ccccn2)cc2ccc(F)c(C#C)c12]

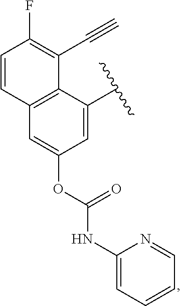 [SMILES: *c1cc(OC(=O)Nc2ccccn2)cc2ccc(F)c(C#C)c12]

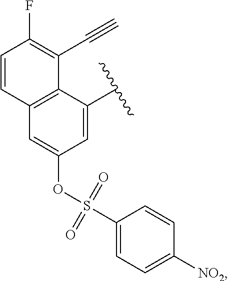 [SMILES: *c1cc(OS(=O)(=O)c2ccc([N+](=O)[O-])cc2)cc2ccc(F)c(C#C)c12]

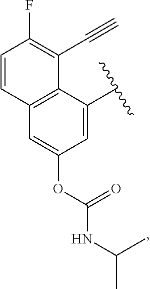 [SMILES: *c1cc(OC(=O)NC(C)C)cc2ccc(F)c(C#C)c12]

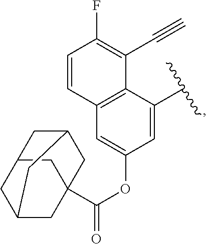 [SMILES: *c1cc(OC(=O)C23CC4CC(CC(C4)C2)C3)cc2ccc(F)c(C#C)c12]

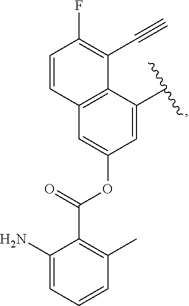 [SMILES: *c1cc(OC(=O)c2c(C)cccc2N)cc2ccc(F)c(C#C)c12]

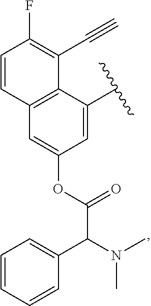 [SMILES: *c1cc(OC(=O)C(c2ccccc2)N(C)C)cc2ccc(F)c(C#C)c12]

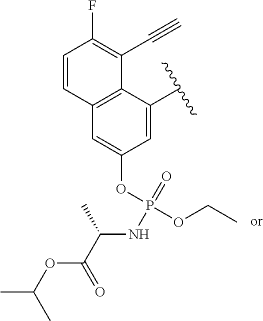 [SMILES: *c1cc(OP(=O)(N[C@@H](C)C(=O)OC(C)C)OCC)cc2ccc(F)c(C#C)c12]

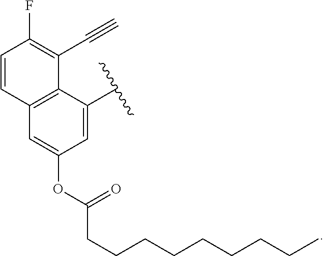 [SMILES: *c1cc(OC(=O)CCCCCCCCC)cc2ccc(F)c(C#C)c12]

[35]. The compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of any one of [1] to [34], wherein, the conjugated form thereof is a PROTAC molecule.

[36]. The compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of any one of [1] to [35] selected from any compound in the Table 13:

TABLE 13

| ID | IUPAC Name |
|---|---|
| 1 | 4-(4-(cyclopropyl(methyl)amino)-8-fluoro-2-(((2R,7aS)-2-fluorotetrahydro-1H-pyrrolizin-7a(5H)-yl) |
| | methoxy)pyrido[4,3-d]pyrimidin-7-yl)-5-ethynyl-6-fluoronaphthalen-2-ol 2,2,2-trifluoroacetic acid; |
| 2 | 4-(4-(cyclopropyl(methyl)amino)-8-fluoro-2-(((2R,7aS)-2-fluorotetrahydro-1H-pyrrolizin-7a(5H)- |
| | yl)methoxy)pyrido[4,3-d]pyrimidin-7-yl)-5-ethynyl-6-fluoronaphthalen-2-ol; |
| 3 | 4-(4-(cyclopropyl(methyl)amino)-8-fluoro-2-(((2R,7aS)-2-fluorotetrahydro-1H-pyrrolizin-7a(5H)- |
| | yl)methoxy)-5-methoxypyrido[4,3-d]pyrimidin-7-yl)-5-ethynyl-6-fluoronaphthalen-2-ol |
| | 2,2,2-trifluoroacetic acid; |
| 4 | 4-(4-(cyclopropyl(methyl)amino)-8-fluoro-2-(((2R,7aS)-2-fluorotetrahydro-1H-pyrrolizin-7a(5H)- |
| | yl)methoxy)-5-methoxypyrido[4,3-d]pyrimidin-7-yl)-5-ethynyl-6-fluoronaphthalen-2-ol; |
| 5 | 5-ethynyl-6-fluoro-4-(8-fluoro-4-((trans-2-fluorocyclopropyl)(methyl)amino)-2-(((2R,7aS)-2-fluorotetrahydro- |
| | 1H-pyrrolizin-7a(5H)-yl)methoxy)pyrido[4,3-d]pyrimidin-7-yl)naphthalen-2-ol |
| | 2,2,2-trifluoroacetic acid; |
| 6 | 5-ethynyl-6-fluoro-4-(8-fluoro-4-((trans-2-fluorocyclopropyl)(methyl)amino)-2-(((2R,7aS)-2-fluorotetrahydro- |
| | 1H-pyrrolizin-7a(5H)-yl)methoxy)pyrido[4,3-d]pyrimidin-7-yl)naphthalen-2-ol; |
| 7 | 5-ethynyl-6-fluoro-4-(8-fluoro-4-(((1S,2S)-2-fluorocyclopropyl)(methyl)amino)-2-(((2R,7aS)-2- |
| | fluorotetrahydro-1H-pyrrolizin-7a(5H)-yl)methoxy)pyrido[4,3-d]pyrimidin-7-yl)naphthalen-2-ol |
| | 2,2,2-trifluoroacetic acid; |
| 8 | 5-ethynyl-6-fluoro-4-(8-fluoro-4-(((1R,2R)-2-fluorocyclopropyl)(methyl)amino)-2-(((2R,7aS)-2- |
| | fluorotetrahydro-1H-pyrrolizin-7a(5H)-yl)methoxy)pyrido[4,3-d]pyrimidin-7-yl)naphthalen-2-ol |
| | 2,2,2-trifluoroacetic acid; |
| 9 | 5-ethynyl-6-fluoro-4-(8-fluoro-4-(((1R,2R)-2-fluorocyclopropyl)(methyl)amino)-2-(((2R,7aS)-2- |
| | fluorotetrahydro-1H-pyrrolizin-7a(5H)-yl)methoxy)pyrido[4,3-d]pyrimidin-7-yl)naphthalen-2-ol; |
| 10 | 5-ethynyl-6-fluoro-4-(8-fluoro-4-(((1S,2S)-2-fluorocyclopropyl)(methyl)amino)-2-(((2R,7aS)-2- |
| | fluorotetrahydro-1H-pyrrolizin-7a(5H)-yl)methoxy)pyrido[4,3-d]pyrimidin-7-yl)naphthalen-2-ol; |
| 11 | 5-ethynyl-6-fluoro-4-(8-fluoro-4-((2-fluorocyclopropyl)(methyl)amino)-2-(((2R,7aS)-2-fluorotetrahydro- |
| | 1H-pyrrolizin-7a(5H)-yl)methoxy)pyrido[4,3-d]pyrimidin-7-yl)naphthalen-2-ol; |
| 12 | 4-(4-(cyclopropyl(methyl-d3)amino)-8-fluoro-2-(((2R,7aS)-2-fluorotetrahydro-1H-pyrrolizin-7a(5H)- |
| | yl)methoxy)pyrido[4,3-d]pyrimidin-7-yl)-5-ethynyl-6-fluoronaphthalen-2-ol 2,2,2-trifluoroacetic acid; |
| 13 | 4-(4-(cyclopropyl(methyl-d3)amino)-8-fluoro-2-(((2R,7aS)-2-fluorotetrahydro-1H-pyrrolizin-7a(5H)- |
| | yl)methoxy)pyrido[4,3-d]pyrimidin-7-yl)-5-ethynyl-6-fluoronaphthalen-2-ol; |
| 14 | 5-ethynyl-6-fluoro-4-(8-fluoro-4-((trans-2-fluorocyclopropyl)(methyl)amino)-2-(((2R,7aS)-2- |
| | methoxytetrahydro-1H-pyrrolizin-7a(5H)-yl)methoxy)pyrido[4,3-d]pyrimidin-7-yl)naphthalen-2-ol |
| | 2,2,2-trifluoroacetic acid; |
| 15 | 5-ethynyl-6-fluoro-4-(8-fluoro-4-((trans-2-fluorocyclopropyl)(methyl)amino)-2-(((2R,7aS)-2- |
| | methoxytetrahydro-1H-pyrrolizin-7a(5H)-yl)methoxy)pyrido[4,3-d]pyrimidin-7-yl)naphthalen-2-ol; |
| 16 | 5-ethynyl-6-fluoro-4-(8-fluoro-4-(((1R,2R)-2-fluorocyclopropyl)(methyl)amino)-2-(((2R,7aS)-2- |
| | methoxytetrahydro-1H-pyrrolizin-7a(5H)-yl)methoxy)pyrido[4,3-d]pyrimidin-7-yl)naphthalen-2-ol |
| | 2,2,2-trifluoroacetic acid; |
| 17 | 5-ethynyl-6-fluoro-4-(8-fluoro-4-(((1R,2R)-2-fluorocyclopropyl)(methyl)amino)-2-(((2R,7aS)-2- |
| | methoxytetrahydro-1H-pyrrolizin-7a(5H)-yl)methoxy)pyrido[4,3-d]pyrimidin-7-yl)naphthalen-2-ol; |
| 18 | 5-ethynyl-6-fluoro-4-(8-fluoro-4-(((1S,2S)-2-fluorocyclopropyl)(methyl)amino)-2-(((2R,7aS)-2- |
| | methoxytetrahydro-1H-pyrrolizin-7a(5H)-yl)methoxy)pyrido[4,3-d]pyrimidin-7-yl)naphthalen-2-ol |
| | 2,2,2-trifluoroacetic acid; |
| 19 | 5-ethynyl-6-fluoro-4-(8-fluoro-4-(((1S,2S)-2-fluorocyclopropyl)(methyl)amino)-2-(((2R,7aS)-2- |
| | methoxytetrahydro-1H-pyrrolizin-7a(5H)-yl)methoxy)pyrido[4,3-d]pyrimidin-7-yl)naphthalen-2-ol; |
| 20 | 5-ethynyl-6-fluoro-4-(8-fluoro-4-((2-fluorocyclopropyl)(methyl)amino)-2-(((2R,7aS)-2- |
| | methoxytetrahydro-1H-pyrrolizin-7a(5H)-yl)methoxy)pyrido[4,3-d]pyrimidin-7-yl)naphthalen-2-ol; |
| 21 | 4-(4-(ethyl(trans-2-fluorocyclopropyl)amino)-8-fluoro-2-(((2R,7aS)-2-fluorotetrahydro-1H-pyrrolizin- |
| | 7a(5H)-yl)methoxy)pyrido[4,3-d]pyrimidin-7-yl)-5-ethynyl-6-fluoronaphthalen-2-ol |
| | 2,2,2-trifluoroacetic acid; |
| 22 | 4-(4-(ethyl(trans-2-fluorocyclopropyl)amino)-8-fluoro-2-(((2R,7aS)-2-fluorotetrahydro-1H-pyrrolizin- |
| | 7a(5H)-yl)methoxy)pyrido[4,3-d]pyrimidin-7-yl)-5-ethynyl-6-fluoronaphthalen-2-ol; |
| 23 | 4-(4-(ethyl((1R,2R)-2-fluorocyclopropyl)amino)-8-fluoro-2-(((2R,7aS)-2-fluorotetrahydro-1H-pyrrolizin- |
| | 7a(5H)-yl)methoxy)pyrido[4,3-d]pyrimidin-7-yl)-5-ethynyl-6-fluoronaphthalen-2-ol |
| | 2,2,2-trifluoroacetic acid; |
| 24 | 4-(4-(ethyl((1R,2R)-2-fluorocyclopropyl)amino)-8-fluoro-2-(((2R,7aS)-2-fluorotetrahydro-1H-pyrrolizin- |
| | 7a(5H)-yl)methoxy)pyrido[4,3-d]pyrimidin-7-yl)-5-ethynyl-6-fluoronaphthalen-2-ol; |
| 25 | 4-(4-(ethyl((1S,2S)-2-fluorocyclopropyl)amino)-8-fluoro-2-(((2R,7aS)-2-fluorotetrahydro-1H-pyrrolizin- |
| | 7a(5H)-yl)methoxy)pyrido[4,3-d]pyrimidin-7-yl)-5-ethynyl-6-fluoronaphthalen-2-ol |
| | 2,2,2-trifluoroacetic acid; |
| 26 | 4-(4-(ethyl((1S,2S)-2-fluorocyclopropyl)amino)-8-fluoro-2-(((2R,7aS)-2-fluorotetrahydro-1H-pyrrolizin- |
| | 7a(5H)-yl)methoxy)pyrido[4,3-d]pyrimidin-7-yl)-5-ethynyl-6-fluoronaphthalen-2-ol; |
| 27 | 4-(4-(ethyl(2-fluorocyclopropyl)amino)-8-fluoro-2-(((2R,7aS)-2-fluorotetrahydro-1H-pyrrolizin-7a(5H)- |
| | yl)methoxy)pyrido[4,3-d]pyrimidin-7-yl)-5-ethynyl-6-fluoronaphthalen-2-ol; |
| 28 | 5-ethynyl-6-fluoro-4-(8-fluoro-4-(((1S,2S)-2-fluorocyclopropyl)(methyl-d3)amino)-2-(((2R,7aS)-2- |
| | fluorotetrahydro-1H-pyrrolizin-7a(5H)-yl)methoxy)pyrido[4,3-d]pyrimidin-7-yl)naphthalen-2-ol |
| | 2,2,2-trifluoroacetic acid; |
| 29 | 5-ethynyl-6-fluoro-4-(8-fluoro-4-(((1S,2S)-2-fluorocyclopropyl)(methyl-d3)amino)-2-(((2R,7aS)-2- |
| | fluorotetrahydro-1H-pyrrolizin-7a(5H)-yl)methoxy)pyrido[4,3-d]pyrimidin-7-yl)naphthalen-2-ol; |
| 30 | 5-ethynyl-6-fluoro-4-(8-fluoro-4-((2-fluorocyclopropyl)(methyl-d3)amino)-2-(((2R,7aS)-2- |
| | fluorotetrahydro-1H-pyrrolizin-7a(5H)-yl)methoxy)pyrido[4,3-d]pyrimidin-7-yl)naphthalen-2-ol; |
| 31 | 4-(4-(cyclopropyl(methyl-d3)amino)-8-fluoro-2-(((2R,7aS)-2-fluorotetrahydro-1H-pyrrolizin-7a(5H)- |
| | yl)methoxy)pyrido[4,3-d]pyrimidin-7-yl)-5-ethynyl-6-fluoronaphthalen-2-yl methylcarbamate; |
| 32 | (2R,7aS)-7a-(((7-(8-ethynyl-7-fluoro-3-hydroxynaphthalen-1-yl)-8-fluoro-4-((trans-2- |
| | fluorocyclopropyl)(methyl)amino)pyrido[4,3-d]pyrimidin-2-yl)oxy)methyl)hexahydro-1H-pyrrolizin-2-ol |
| | 2,2,2-trifluoroacetic acid; |
| 33 | (2R,7aS)-7a-(((7-(8-ethynyl-7-fluoro-3-hydroxynaphthalen-1-yl)-8-fluoro-4-((trans-2- |
| | fluorocyclopropyl)(methyl)amino)pyrido[4,3-d]pyrimidin-2-yl)oxy)methyl)hexahydro-1H-pyrrolizin-2-ol; |
| 34 | (2R,7aS)-7a-(((7-(8-ethynyl-7-fluoro-3-hydroxynaphthalen-1-yl)-8-fluoro-4-(((1R,2R)-2- |
| | fluorocyclopropyl)(methyl)amino)pyrido[4,3-d]pyrimidin-2-yl)oxy)methyl)hexahydro-1H-pyrrolizin-2-ol |
| | 2,2,2-trifluoroacetic acid; |
| 35 | (2R,7aS)-7a-(((7-(8-ethynyl-7-fluoro-3-hydroxynaphthalen-1-yl)-8-fluoro-4-(((1R,2R)-2- |
| | fluorocyclopropyl)(methyl)amino)pyrido[4,3-d]pyrimidin-2-yl)oxy)methyl)hexahydro-1H-pyrrolizin-2-ol; |
| 36 | (2R,7aS)-7a-(((7-(8-ethynyl-7-fluoro-3-hydroxynaphthalen-1-yl)-8-fluoro-4-(((1S,2S)-2- |
| | fluorocyclopropyl)(methyl)amino)pyrido[4,3-d]pyrimidin-2-yl)oxy)methyl)hexahydro-1H-pyrrolizin-2-ol |
| | 2,2,2-trifluoroacetic acid; |
| 37 | (2R,7aS)-7a-(((7-(8-ethynyl-7-fluoro-3-hydroxynaphthalen-1-yl)-8-fluoro-4-(((1S,2S)-2- |
| | fluorocyclopropyl)(methyl)amino)pyrido[4,3-d]pyrimidin-2-yl)oxy)methyl)hexahydro-1H-pyrrolizin-2-ol; |
| 38 | (2R,7aS)-7a-(((7-(8-ethynyl-7-fluoro-3-hydroxynaphthalen-1-yl)-8-fluoro-4-((2- |
| | fluorocyclopropyl)(methyl)amino)pyrido[4,3-d]pyrimidin-2-yl)oxy)methyl)hexahydro-1H-pyrrolizin-2-ol; |
| 39 | 5-ethynyl-6-fluoro-4-(8-fluoro-2-(((2R,7aS)-2-fluorotetrahydro-1H-pyrrolizin-7a(5H)-yl)methoxy)-4- |
| | ((1-methylcyclopropyl)amino)pyrido[4,3-d]pyrimidin-7-yl)naphthalen-2-ol 2,2,2-trifluoroacetic acid; |
| 40 | 5-ethynyl-6-fluoro-4-(8-fluoro-2-(((2R,7aS)-2-fluorotetrahydro-1H-pyrrolizin-7a(5H)-yl)methoxy)-4- |
| | ((1-methylcyclopropyl)amino)pyrido[4,3-d]pyrimidin-7-yl)naphthalen-2-ol; |
| 41 | 4-(4-((2,2-difluorocyclopropyl)(methyl)amino)-8-fluoro-2-(((2R,7aS)-2-fluorotetrahydro-1H- |
| | pyrrolizin-7a(5H)-yl)methoxy)pyrido[4,3-d]pyrimidin-7-yl)-5-ethynyl-6-fluoronaphthalen-2-ol |
| | 2,2,2-trifluoroacetic acid; |
| 42 | 4-(4-((2,2-difluorocyclopropyl)(methyl)amino)-8-fluoro-2-(((2R,7aS)-2-fluorotetrahydro-1H- |
| | pyrrolizin-7a(5H)-yl)methoxy)pyrido[4,3-d]pyrimidin-7-yl)-5-ethynyl-6-fluoronaphthalen-2-ol; |
| 43 | 5-ethynyl-6-fluoro-4-(8-fluoro-4-(((1S,2R)-2-fluorocyclopropyl)(methyl)amino)-2-(((2R,7aS)-2- |
| | fluorotetrahydro-1H-pyrrolizin-7a(5H)-yl)methoxy)pyrido[4,3-d]pyrimidin-7-yl)naphthalen-2-ol |
| | 2,2,2-trifluoroacetic acid; |
| 44 | 5-ethynyl-6-fluoro-4-(8-fluoro-4-(((1S,2R)-2-fluorocyclopropyl)(methyl)amino)-2-(((2R,7aS)-2- |
| | fluorotetrahydro-1H-pyrrolizin-7a(5H)-yl)methoxy)pyrido[4,3-d]pyrimidin-7-yl)naphthalen-2-ol; |
| 45 | 5-ethynyl-6-fluoro-4-(8-fluoro-4-(((1R,2S)-2-fluorocyclopropyl)(methyl)amino)-2-(((2R,7aS)-2- |
| | fluorotetrahydro-1H-pyrrolizin-7a(5H)-yl)methoxy)pyrido[4,3-d]pyrimidin-7-yl)naphthalen-2-ol |
| | 2,2,2-trifluoroacetic acid; |
| 46 | 5-ethynyl-6-fluoro-4-(8-fluoro-4-(((1R,2S)-2-fluorocyclopropyl)(methyl)amino)-2-(((2R,7aS)-2- |
| | fluorotetrahydro-1H-pyrrolizin-7a(5H)-yl)methoxy)pyrido[4,3-d]pyrimidin-7-yl)naphthalen-2-ol; |
| 47 | 4-(4-(cyclopropyl(cyclopropylmethyl)amino)-8-fluoro-2-(((2R,7aS)-2-fluorotetrahydro-1H- |
| | pyrrolizin-7a(5H)-yl)methoxy)pyrido[4,3-d]pyrimidin-7-yl)-5-ethynyl-6-fluoronaphthalen-2-ol |
| | 2,2,2-trifluoroacetic acid; |
| 48 | 4-(4-(cyclopropyl(cyclopropylmethyl)amino)-8-fluoro-2-(((2R,7aS)-2-fluorotetrahydro-1H- |
| | pyrrolizin-7a(5H)-yl)methoxy)pyrido[4,3-d]pyrimidin-7-yl)-5-ethynyl-6-fluoronaphthalen-2-ol; |
| 49 | 6-fluoro-4-(8-fluoro-4-((trans-2-fluorocyclopropyl)(methyl)amino)-2-(((2R,7aS)-2-fluorotetrahydro- |
| | 1H-pyrrolizin-7a(5H)-yl)methoxy)pyrido[4,3-d]pyrimidin-7-yl)-5-vinylnaphthalen-2-ol |
| | 2,2,2-trifluoroacetic acid; |
| 50 | 6-fluoro-4-(8-fluoro-4-((trans-2-fluorocyclopropyl)(methyl)amino)-2-(((2R,7aS)-2-fluorotetrahydro- |
| | 1H-pyrrolizin-7a(5H)-yl)methoxy)pyrido[4,3-d]pyrimidin-7-yl)-5-vinylnaphthalen-2-ol; |
| 51 | 6-fluoro-4-(8-fluoro-4-(((1S,2S)-2-fluorocyclopropyl)(methyl)amino)-2-(((2R,7aS)-2-fluorotetrahydro- |
| | 1H-pyrrolizin-7a(5H)-yl)methoxy)pyrido[4,3-d]pyrimidin-7-yl)-5-vinylnaphthalen-2-ol |
| | 2,2,2-trifluoroacetic acid; |
| 52 | 6-fluoro-4-(8-fluoro-4-(((1S,2S)-2-fluorocyclopropyl)(methyl)amino)-2-(((2R,7aS)-2-fluorotetrahydro- |
| | 1H-pyrrolizin-7a(5H)-yl)methoxy)pyrido[4,3-d]pyrimidin-7-yl)-5-vinylnaphthalen-2-ol; |
| 53 | 6-fluoro-4-(8-fluoro-4-(((1R,2R)-2-fluorocyclopropyl)(methyl)amino)-2-(((2R,7aS)-2-fluorotetrahydro- |
| | 1H-pyrrolizin-7a(5H)-yl)methoxy)pyrido[4,3-d]pyrimidin-7-yl)-5-vinylnaphthalen-2-ol |
| | 2,2,2-trifluoroacetic acid; |
| 54 | 6-fluoro-4-(8-fluoro-4-(((1R,2R)-2-fluorocyclopropyl)(methyl)amino)-2-(((2R,7aS)-2-fluorotetrahydro- |
| | 1H-pyrrolizin-7a(5H)-yl)methoxy)pyrido[4,3-d]pyrimidin-7-yl)-5-vinylnaphthalen-2-ol; |
| 55 | 6-fluoro-4-(8-fluoro-4-((2-fluorocyclopropyl)(methyl)amino)-2-(((2R,7aS)-2-fluorotetrahydro-1H- |
| | pyrrolizin-7a(5H)-yl)methoxy)pyrido[4,3-d]pyrimidin-7-yl)-5-vinylnaphthalen-2-ol; |
| 56 | 5-ethyl-6-fluoro-4-(8-fluoro-4-((trans-2-fluorocyclopropyl)(methyl)amino)-2-(((2R,7aS)-2- |
| | fluorotetrahydro-1H-pyrrolizin-7a(5H)-yl)methoxy)pyrido[4,3-d]pyrimidin-7-yl)naphthalen-2-ol |
| | 2,2,2-trifluoroacetic acid; |
| 57 | 5-ethyl-6-fluoro-4-(8-fluoro-4-((trans-2-fluorocyclopropyl)(methyl)amino)-2-(((2R,7aS)-2- |
| | fluorotetrahydro-1H-pyrrolizin-7a(5H)-yl)methoxy)pyrido[4,3-d]pyrimidin-7-yl)naphthalen-2-ol; |
| 58 | 5-ethyl-6-fluoro-4-(8-fluoro-4-(((1S,2S)-2-fluorocyclopropyl)(methyl)amino)-2-(((2R,7aS)-2- |
| | fluorotetrahydro-1H-pyrrolizin-7a(5H)-yl)methoxy)pyrido[4,3-d]pyrimidin-7-yl)naphthalen-2-ol |
| | 2,2,2-trifluoroacetic acid; |
| 59 | 5-ethyl-6-fluoro-4-(8-fluoro-4-(((1S,2S)-2-fluorocyclopropyl)(methyl)amino)-2-(((2R,7aS)-2- |
| | fluorotetrahydro-1H-pyrrolizin-7a(5H)-yl)methoxy)pyrido[4,3-d]pyrimidin-7-yl)naphthalen-2-ol; |
| 60 | 5-ethyl-6-fluoro-4-(8-fluoro-4-(((1R,2R)-2-fluorocyclopropyl)(methyl)amino)-2-(((2R,7aS)-2- |
| | fluorotetrahydro-1H-pyrrolizin-7a(5H)-yl)methoxy)pyrido[4,3-d]pyrimidin-7-yl)naphthalen-2-ol |
| | 2,2,2-trifluoroacetic acid; |
| 61 | 5-ethyl-6-fluoro-4-(8-fluoro-4-(((1R,2R)-2-fluorocyclopropyl)(methyl)amino)-2-(((2R,7aS)-2- |
| | fluorotetrahydro-1H-pyrrolizin-7a(5H)-yl)methoxy)pyrido[4,3-d]pyrimidin-7-yl)naphthalen-2-ol; |
| 62 | 5-ethyl-6-fluoro-4-(8-fluoro-4-((2-fluorocyclopropyl)(methyl)amino)-2-(((2R,7aS)-2- |
| | fluorotetrahydro-1H-pyrrolizin-7a(5H)-yl)methoxy)pyrido[4,3-d]pyrimidin-7-yl)naphthalen-2-ol; |
| 63 | 4-(4-(cyclopropyl(2,2-difluoroethyl)amino)-8-fluoro-2-(((2R,7aS)-2-fluorotetrahydro-1H-pyrrolizin- |
| | 7a(5H)-yl)methoxy)pyrido[4,3-d]pyrimidin-7-yl)-5-ethynyl-6-fluoronaphthalen-2-ol |
| | 2,2,2-trifluoroacetic acid; |
| 64 | 4-(4-(cyclopropyl(2,2-difluoroethyl)amino)-8-fluoro-2-(((2R,7aS)-2-fluorotetrahydro-1H-pyrrolizin-7 |
| | a(5H)-yl)methoxy)pyrido[4,3-d]pyrimidin-7-yl)-5-ethynyl-6-fluoronaphthalen-2-ol; |
| 65 | 4-(4-(cyclopropylamino)-8-fluoro-2-(((2R,7aS)-2-fluorotetrahydro-1H-pyrrolizin-7a(5H)- |
| | yl)methoxy)pyrido[4,3-d]pyrimidin-7-yl)-5-ethynyl-6-fluoronaphthalen-2-ol formic acid; |
| 66 | 4-(4-(cyclopropylamino)-8-fluoro-2-(((2R,7aS)-2-fluorotetrahydro-1H-pyrrolizin-7a(5H)- |
| | yl)methoxy)pyrido[4,3-d]pyrimidin-7-yl)-5-ethynyl-6-fluoronaphthalen-2-ol; |
| 67 | 5-ethynyl-6-fluoro-4-(8-fluoro-2-(((2R,7aS)-2-fluorotetrahydro-1H-pyrrolizin-7a(5H)-yl)methoxy)-4- |
| | (methyl(1-methylcyclopropyl)amino)pyrido[4,3-d]pyrimidin-7-yl)naphthalen-2-ol; |
| 68 | 5-ethynyl-6-fluoro-4-(8-fluoro-4-(((1S,2S)-2-fluorocyclopropyl)(methyl)amino)-2-(((2R,7aS)-2- |
| | fluorotetrahydro-1H-pyrrolizin-7a(5H)-yl)methoxy)pyrido[4,3-d]pyrimidin-7-yl)naphthalen-2-yl |
| | methylcarbamate 2,2,2-trifluoroacetic acid; |
| 69 | 5-ethynyl-6-fluoro-4-(8-fluoro-4-(((1S,2S)-2-fluorocyclopropyl)(methyl)amino)-2-(((2R,7aS)-2- |
| | fluorotetrahydro-1H-pyrrolizin-7a(5H)-yl)methoxy)pyrido[4,3-d]pyrimidin-7-yl)naphthalen-2-yl |
| | methylcarbamate; |
| 70 | 5-ethynyl-6-fluoro-4-(8-fluoro-4-((2-fluorocyclopropyl)(methyl)amino)-2-(((2R,7aS)-2-fluorotetrahydro- |
| | 1H-pyrrolizin-7a(5H)-yl)methoxy)pyrido[4,3-d]pyrimidin-7-yl)naphthalen-2-yl methylcarbamate; |
| 71 | 5-ethynyl-6-fluoro-4-(8-fluoro-4-(((1S,2S)-2-fluorocyclopropyl)(methyl)amino)-2-(((2R,7aS)-2- |
| | fluorotetrahydro-1H-pyrrolizin-7a(5H)-yl)methoxy)pyrido[4,3-d]pyrimidin-7-yl)naphthalen-2-yl |
| | dimethylcarbamate; |
| 72 | 5-ethynyl-6-fluoro-4-(8-fluoro-4-(((1S,2S)-2-fluorocyclopropyl)(methyl)amino)-2-(((2R,7aS)-2- |
| | fluorotetrahydro-1H-pyrrolizin-7a(5H)-yl)methoxy)pyrido[4,3-d]pyrimidin-7-yl)naphthalen-2-yl |
| | dimethylcarbamate 2,2,2-trifluoroacetic acid; |
| 73 | 5-ethynyl-6-fluoro-4-(8-fluoro-4-((2-fluorocyclopropyl)(methyl)amino)-2-(((2R,7aS)-2- |
| | fluorotetrahydro-1H-pyrrolizin-7a(5H)-yl)methoxy)pyrido[4,3-d]pyrimidin-7-yl)naphthalen-2-yl |
| | dimethylcarbamate; |
| 74 | 5-ethynyl-6-fluoro-4-(8-fluoro-2-(((2R,7aS)-2-fluorotetrahydro-1H-pyrrolizin-7a(5H)-yl)methoxy)-4- |
| | ((2-methylcyclopropyl)amino)pyrido[4,3-d]pyrimidin-7-yl)naphthalen-2-ol; |
| 75 | ((5-ethynyl-6-fluoro-4-(8-fluoro-4-((trans-2-fluorocyclopropyl)(methyl)amino)-2-(((2R,7aS)-2- |
| | fluorotetrahydro-1H-pyrrolizin-7a(5H)-yl)methoxy)pyrido[4,3-d]pyrimidin-7-yl)naphthalen-2- |
| | yl)oxy)methyl dihydrogen phosphate 2,2,2-trifluoroacetic acid; |
| 76 | ((5-ethynyl-6-fluoro-4-(8-fluoro-4-((trans-2-fluorocyclopropyl)(methyl)amino)-2-(((2R,7aS)-2- |
| | fluorotetrahydro-1H-pyrrolizin-7a(5H)-yl)methoxy)pyrido[4,3-d]pyrimidin-7-yl)naphthalen-2-yl)oxy)methyl |
| | dihydrogen phosphate; |
| 77 | ((5-ethynyl-6-fluoro-4-(8-fluoro-4-(((1S,2S)-2-fluorocyclopropyl)(methyl)amino)-2-(((2R,7aS)-2- |
| | fluorotetrahydro-1H-pyrrolizin-7a(5H)-yl)methoxy)pyrido[4,3-d]pyrimidin-7-yl)naphthalen-2-yl)oxy)methyl |
| | dihydrogen phosphate 2,2,2-trifluoroacetic acid; |
| 78 | ((5-ethynyl-6-fluoro-4-(8-fluoro-4-(((1S,2S)-2-fluorocyclopropyl)(methyl)amino)-2-(((2R,7aS)-2- |
| | fluorotetrahydro-1H-pyrrolizin-7a(5H)-yl)methoxy)pyrido[4,3-d]pyrimidin-7-yl)naphthalen-2-yl)oxy)methyl |
| | dihydrogen phosphate; |
| 79 | ((5-ethynyl-6-fluoro-4-(8-fluoro-4-(((1R,2R)-2-fluorocyclopropyl)(methyl)amino)-2-(((2R,7aS)-2- |
| | fluorotetrahydro-1H-pyrrolizin-7a(5H)-yl)methoxy)pyrido[4,3-d]pyrimidin-7-yl)naphthalen-2-yl)oxy)methyl |
| | dihydrogen phosphate 2,2,2-trifluoroacetic acid; |
| 80 | ((5-ethynyl-6-fluoro-4-(8-fluoro-4-(((1R,2R)-2-fluorocyclopropyl)(methyl)amino)-2-(((2R,7aS)-2- |
| | fluorotetrahydro-1H-pyrrolizin-7a(5H)-yl)methoxy)pyrido[4,3-d]pyrimidin-7-yl)naphthalen-2-yl)oxy)methyl |
| | dihydrogen phosphate; |
| 81 | ((5-ethynyl-6-fluoro-4-(8-fluoro-4-((2-fluorocyclopropyl)(methyl)amino)-2-(((2R,7aS)-2-fluorotetrahydro- |
| | 1H-pyrrolizin-7a(5H)-yl)methoxy)pyrido[4,3-d]pyrimidin-7-yl)naphthalen-2-yl)oxy)methyl |
| | dihydrogen phosphate; |
| 82 | Di-tert-butyl |
| | (((5-ethynyl-6-fluoro-4-(8-fluoro-4-((trans-2-fluorocyclopropyl)(methyl)amino)-2-(((2R,7aS)-2- |
| | fluorotetrahydro-1H-pyrrolizin-7a(5H)-yl)methoxy)pyrido[4,3-d]pyrimidin-7-yl)naphthalen-2-yl)oxy)methyl) |
| | phosphate 2,2,2-trifluoroacetic acid; |
| 83 | Di-tert-butyl |
| | (((5-ethynyl-6-fluoro-4-(8-fluoro-4-((trans-2-fluorocyclopropyl)(methyl)amino)-2-(((2R,7aS)-2- |
| | fluorotetrahydro-1H-pyrrolizin-7a(5H)-yl)methoxy)pyrido[4,3-d]pyrimidin-7-yl)naphthalen-2- |
| | yl)oxy)methyl) phosphate; |
| 84 | Di-tert-butyl |
| | (((5-ethynyl-6-fluoro-4-(8-fluoro-4-(((1S,2S)-2-fluorocyclopropyl)(methyl)amino)-2-(((2R,7aS)-2- |
| | fluorotetrahydro-1H-pyrrolizin-7a(5H)-yl)methoxy)pyrido[4,3-d]pyrimidin-7-yl)naphthalen-2- |
| | yl)oxy)methyl) phosphate 2,2,2-trifluoroacetic acid; |
| 85 | Di-tert-butyl |
| | (((5-ethynyl-6-fluoro-4-(8-fluoro-4-(((1S,2S)-2-fluorocyclopropyl)(methyl)amino)-2-(((2R,7aS)-2- |
| | fluorotetrahydro-1H-pyrrolizin-7a(5H)-yl)methoxy)pyrido[4,3-d]pyrimidin-7-yl)naphthalen-2- |
| | yl)oxy)methyl) phosphate; |
| 86 | Di-tert-butyl |
| | (((5-ethynyl-6-fluoro-4-(8-fluoro-4-(((1R,2R)-2-fluorocyclopropyl)(methyl)amino)-2-(((2R,7aS)-2- |
| | fluorotetrahydro-1H-pyrrolizin-7a(5H)-yl)methoxy)pyrido[4,3-d]pyrimidin-7-yl)naphthalen-2- |
| | yl)oxy)methyl) phosphate 2,2,2-trifluoroacetic acid; |
| 87 | Di-tert-butyl |
| | (((5-ethynyl-6-fluoro-4-(8-fluoro-4-(((1R,2R)-2-fluorocyclopropyl)(methyl)amino)-2-(((2R,7aS)-2- |
| | fluorotetrahydro-1H-pyrrolizin-7a(5H)-yl)methoxy)pyrido[4,3-d]pyrimidin-7-yl)naphthalen-2- |
| | yl)oxy)methyl) phosphate; |
| 88 | Di-tert-butyl |
| | (((5-ethynyl-6-fluoro-4-(8-fluoro-4-((2-fluorocyclopropyl)(methyl)amino)-2-(((2R,7aS)-2-fluorotetrahydro- |
| | 1H-pyrrolizin-7a(5H)-yl)methoxy)pyrido[4,3-d]pyrimidin-7-yl)naphthalen-2-yl)oxy)methyl) phosphate; |
| 89 | 5-ethynyl-6-fluoro-4-(8-fluoro-4-((1-(fluoromethyl)cyclopropyl)amino)-2-(((2R,7aS)-2-fluorotetrahydro- |
| | 1H-pyrrolizin-7a(5H)-yl)methoxy)pyrido[4,3-d]pyrimidin-7-yl)naphthalen-2-ol; |
| 90 | 4-(4-(cyclopropyl(methyl-d3)amino)-8-fluoro-2-(((2R,7aS)-2-fluorotetrahydro-1H-pyrrolizin-7a(5H)- |
| | yl-2,5,5-d3)methoxy-d2)pyrido[4,3-d]pyrimidin-7-yl)-5-ethynyl-6-fluoronaphthalen-2-ol |
| | 2,2,2-trifluoroacetic acid; |
| 91 | 4-(4-(cyclopropyl(methyl-d3)amino)-8-fluoro-2-(((2R,7aS)-2-fluorotetrahydro-1H-pyrrolizin-7a(5H)- |
| | yl-2,5,5-d3)methoxy-d2)pyrido[4,3-d]pyrimidin-7-yl)-5-ethynyl-6-fluoronaphthalen-2-ol; |
| 92 | 4-(5-chloro-4-(cyclopropyl(methyl)amino)-8-fluoro-2-(((2R,7aS)-2-fluorotetrahydro-1H-pyrrolizin-7a(5H)- |
| | yl)methoxy)pyrido[4,3-d]pyrimidin-7-yl)-5-ethynyl-6-fluoronaphthalen-2-ol 2,2,2-trifluoroacetic acid; |
| 93 | 4-(5-chloro-4-(cyclopropyl(methyl)amino)-8-fluoro-2-(((2R,7aS)-2-fluorotetrahydro-1H-pyrrolizin-7a(5H)- |
| | yl)methoxy)pyrido[4,3-d]pyrimidin-7-yl)-5-ethynyl-6-fluoronaphthalen-2-ol; |
| 94 | 4-(4-(cyclopropylamino)-8-fluoro-2-(((2R,7aS)-2-fluorotetrahydro-1H-pyrrolizin-7a(5H)-yl)methoxy)-5- |
| | methoxypyrido[4,3-d]pyrimidin-7-yl)-5-ethynyl-6-fluoronaphthalen-2-ol; |
| 95 | 4-(cyclopropyl(methyl)amino)-7-(8-ethynyl-7-fluoro-3-hydroxynaphthalen-1-yl)-8-fluoro-2-(((2R,7aS)- |
| | 2-fluorotetrahydro-1H-pyrrolizin-7a(5H)-yl)methoxy)pyrido[4,3-d]pyrimidine-5-carbonitrile; |
| 96 | 4-(4-(cyclopropyl(2-methoxyethyl)amino)-8-fluoro-2-(((2R,7aS)-2-fluorotetrahydro-1H-pyrrolizin-7a(5H)- |
| | yl)methoxy)pyrido[4,3-d]pyrimidin-7-yl)-5-ethynyl-6-fluoronaphthalen-2-ol |
| | 2,2,2-trifluoroacetic acid; |
| 97 | 4-(4-(cyclopropyl(2-methoxyethyl)amino)-8-fluoro-2-(((2R,7aS)-2-fluorotetrahydro-1H-pyrrolizin-7a(5H)- |
| | yl)methoxy)pyrido[4,3-d]pyrimidin-7-yl)-5-ethynyl-6-fluoronaphthalen-2-ol; |
| 98 | 4-(4-(cyclopropyl(2-fluoroethyl)amino)-8-fluoro-2-(((2R,7aS)-2-fluorotetrahydro-1H-pyrrolizin-7a(5H)- |
| | yl)methoxy)pyrido[4,3-d]pyrimidin-7-yl)-5-ethynyl-6-fluoronaphthalen-2-ol 2,2,2-trifluoroacetic acid; |
| 99 | 4-(4-(cyclopropyl(2-fluoroethyl)amino)-8-fluoro-2-(((2R,7aS)-2-fluorotetrahydro-1H-pyrrolizin-7a(5H)- |
| | yl)methoxy)pyrido[4,3-d]pyrimidin-7-yl)-5-ethynyl-6-fluoronaphthalen-2-ol; |
| 100 | Ethyl |
| | N-cyclopropyl-N-(7-(8-ethynyl-7-fluoro-3-hydroxynaphthalen-1-yl)-8-fluoro-2-(((2R,7aS)-2- |
| | fluorotetrahydro-1H-pyrrolizin-7a(5H)-yl)methoxy)pyrido[4,3-d]pyrimidin-4-yl)glycinate |
| | 2,2,2-trifluoroacetic acid; |
| 101 | Ethyl |
| | N-cyclopropyl-N-(7-(8-ethynyl-7-fluoro-3-hydroxynaphthalen-1-yl)-8-fluoro-2-(((2R,7aS)-2- |
| | fluorotetrahydro-1H-pyrrolizin-7a(5H)-yl)methoxy)pyrido[4,3-d]pyrimidin-4-yl)glycinate; |
| 102 | N-cyclopropyl-N-(7-(8-ethynyl-7-fluoro-3-hydroxynaphthalen-1-yl)-8-fluoro-2-(((2R,7aS)-2- |
| | fluorotetrahydro-1H-pyrrolizin-7a(5H)-yl)methoxy)pyrido[4,3-d]pyrimidin-4-yl)acetamide |
| | 2,2,2-trifluoroacetic acid; |
| 103 | N-cyclopropyl-N-(7-(8-ethynyl-7-fluoro-3-hydroxynaphthalen-1-yl)-8-fluoro-2-(((2R,7aS)-2- |
| | fluorotetrahydro-1H-pyrrolizin-7a(5H)-yl)methoxy)pyrido[4,3-d]pyrimidin-4-yl)acetamide; |
| 104 | 2-(cyclopropyl(7-(8-ethynyl-7-fluoro-3-hydroxynaphthalen-1-yl)-8-fluoro-2-(((2R,7aS)-2- |
| | fluorotetrahydro-1H-pyrrolizin-7a(5H)-yl)methoxy)pyrido[4,3-d]pyrimidin-4-yl)amino)acetonitrile |
| | 2,2,2-trifluoroacetic acid; |
| 105 | 2-(cyclopropyl(7-(8-ethynyl-7-fluoro-3-hydroxynaphthalen-1-yl)-8-fluoro-2-(((2R,7aS)-2- |
| | fluorotetrahydro-1H-pyrrolizin-7a(5H)-yl)methoxy)pyrido[4,3-d]pyrimidin-4-yl)amino)acetonitrile; |
| 106 | 5-ethynyl-6-fluoro-4-(8-fluoro-2-(((2R,7aS)-2-fluorotetrahydro-1H-pyrrolizin-7a(5H)-yl)methoxy)-4- |
| | ((trans-2-methoxycyclopropyl)(methyl)amino)pyrido[4,3-d]pyrimidin-7-yl)naphthalen-2-ol; |
| 107 | 5-ethynyl-6-fluoro-4-(8-fluoro-2-(((2R,7aS)-2-fluorotetrahydro-1H-pyrrolizin-7a(5H)-yl)methoxy)-4- |
| | (((1S,2S)-2-methoxycyclopropyl)(methyl)amino)pyrido[4,3-d]pyrimidin-7-yl)naphthalen-2-ol; |
| 108 | 5-ethynyl-6-fluoro-4-(8-fluoro-2-(((2R,7aS)-2-fluorotetrahydro-1H-pyrrolizin-7a(5H)-yl)methoxy)-4- |
| | (((1R,2R)-2-methoxycyclopropyl)(methyl)amino)pyrido[4,3-d]pyrimidin-7-yl)naphthalen-2-ol; |
| 109 | 5-ethynyl-6-fluoro-4-(8-fluoro-2-(((2R,7aS)-2-fluorotetrahydro-1H-pyrrolizin-7a(5H)-yl)methoxy)-4- |
| | ((2-methoxycyclopropyl)(methyl)amino)pyrido[4,3-d]pyrimidin-7-yl)naphthalen-2-ol; |
| 110 | 5-ethynyl-6-fluoro-4-(8-fluoro-2-(((2R,7aS)-2-fluorotetrahydro-1H-pyrrolizin-7a(5H)-yl)methoxy)-4- |
| | ((trans-2-(methoxymethyl)cyclopropyl)(methyl)amino)pyrido[4,3-d]pyrimidin-7-yl)naphthalen-2-ol |
| | 2,2,2-trifluoroacetic acid; |
| 111 | 5-ethynyl-6-fluoro-4-(8-fluoro-2-(((2R,7aS)-2-fluorotetrahydro-1H-pyrrolizin-7a(5H)-yl)methoxy)-4- |
| | ((trans-2-(methoxymethyl)cyclopropyl)(methyl)amino)pyrido[4,3-d]pyrimidin-7-yl)naphthalen-2-ol; |
| 112 | 5-ethynyl-6-fluoro-4-(8-fluoro-2-(((2R,7aS)-2-fluorotetrahydro-1H-pyrrolizin-7a(5H)-yl)methoxy)-4- |
| | (((1R,2R)-2-(methoxymethyl)cyclopropyl)(methyl)amino)pyrido[4,3-d]pyrimidin-7-yl)naphthalen-2-ol |
| | 2,2,2-trifluoroacetic acid; |
| 113 | 5-ethynyl-6-fluoro-4-(8-fluoro-2-(((2R,7aS)-2-fluorotetrahydro-1H-pyrrolizin-7a(5H)-yl)methoxy)-4- |
| | (((1R,2R)-2-(methoxymethyl)cyclopropyl)(methyl)amino)pyrido[4,3-d]pyrimidin-7-yl)naphthalen-2-ol; |
| 114 | 5-ethynyl-6-fluoro-4-(8-fluoro-2-(((2R,7aS)-2-fluorotetrahydro-1H-pyrrolizin-7a(5H)-yl)methoxy)-4- |
| | (((1S,2S)-2-(methoxymethyl)cyclopropyl)(methyl)amino)pyrido[4,3-d]pyrimidin-7-yl)naphthalen-2-ol |
| | 2,2,2-trifluoroacetic acid; |
| 115 | 5-ethynyl-6-fluoro-4-(8-fluoro-2-(((2R,7aS)-2-fluorotetrahydro-1H-pyrrolizin-7a(5H)-yl)methoxy)-4- |
| | (((1S,2S)-2-(methoxymethyl)cyclopropyl)(methyl)amino)pyrido[4,3-d]pyrimidin-7-yl)naphthalen-2-ol; |
| 116 | 5-ethynyl-6-fluoro-4-(8-fluoro-2-(((2R,7aS)-2-fluorotetrahydro-1H-pyrrolizin-7a(5H)-yl)methoxy)-4- |
| | ((2-(methoxymethyl)cyclopropyl)(methyl)amino)pyrido[4,3-d]pyrimidin-7-yl)naphthalen-2-ol; |
| 117 | 5-ethynyl-6-fluoro-4-(8-fluoro-2-(((2R,7aS)-2-fluorotetrahydro-1H-pyrrolizin-7a(5H)-yl)methoxy)-4- |
| | (methyl(trans-2-(trifluoromethyl)cyclopropyl)amino)pyrido[4,3-d]pyrimidin-7-yl)naphthalen-2-ol |
| | 2,2,2-trifluoroacetic acid; |
| 118 | 5-ethynyl-6-fluoro-4-(8-fluoro-2-(((2R,7aS)-2-fluorotetrahydro-1H-pyrrolizin-7a(5H)-yl)methoxy)-4- |
| | (methyl(trans-2-(trifluoromethyl)cyclopropyl)amino)pyrido[4,3-d]pyrimidin-7-yl)naphthalen-2-ol; |
| 119 | 5-ethynyl-6-fluoro-4-(8-fluoro-2-(((2R,7aS)-2-fluorotetrahydro-1H-pyrrolizin-7a(5H)-yl)methoxy)-4- |
| | (methyl((1S,2S)-2-(trifluoromethyl)cyclopropyl)amino)pyrido[4,3-d]pyrimidin-7-yl)naphthalen-2-ol |
| | 2,2,2-trifluoroacetic acid; |
| 120 | 5-ethynyl-6-fluoro-4-(8-fluoro-2-(((2R,7aS)-2-fluorotetrahydro-1H-pyrrolizin-7a(5H)-yl)methoxy)-4- |
| | (methyl((1S,2S)-2-(trifluoromethyl)cyclopropyl)amino)pyrido[4,3-d]pyrimidin-7-yl)naphthalen-2-ol; |
| 121 | 5-ethynyl-6-fluoro-4-(8-fluoro-2-(((2R,7aS)-2-fluorotetrahydro-1H-pyrrolizin-7a(5H)-yl)methoxy)-4- |
| | (methyl((1R,2R)-2-(trifluoromethyl)cyclopropyl)amino)pyrido[4,3-d]pyrimidin-7-yl)naphthalen-2-ol |
| | 2,2,2-trifluoroacetic acid; |
| 122 | 5-ethynyl-6-fluoro-4-(8-fluoro-2-(((2R,7aS)-2-fluorotetrahydro-1H-pyrrolizin-7a(5H)-yl)methoxy)-4- |
| | (methyl((1R,2R)-2-(trifluoromethyl)cyclopropyl)amino)pyrido[4,3-d]pyrimidin-7-yl)naphthalen-2-ol; |
| 123 | 5-ethynyl-6-fluoro-4-(8-fluoro-2-(((2R,7aS)-2-fluorotetrahydro-1H-pyrrolizin-7a(5H)-yl)methoxy)-4- |
| | (methyl(2-(trifluoromethyl)cyclopropyl)amino)pyrido[4,3-d]pyrimidin-7-yl)naphthalen-2-ol; |
| 124 | trans-2-((7-(8-ethynyl-7-fluoro-3-hydroxynaphthalen-1-yl)-8-fluoro-2-(((2R,7aS)-2-fluorotetrahydro- |
| | 1H-pyrrolizin-7a(5H)-yl)methoxy)pyrido[4,3-d]pyrimidin-4-yl)(methyl)amino)cyclopropane-1-carbonitrile |
| | 2,2,2-trifluoroacetic acid; |
| 125 | trans-2-((7-(8-ethynyl-7-fluoro-3-hydroxynaphthalen-1-yl)-8-fluoro-2-(((2R,7aS)-2-fluorotetrahydro- |
| | 1H-pyrrolizin-7a(5H)-yl)methoxy)pyrido[4,3-d]pyrimidin-4-yl)(methyl)amino)cyclopropane-1-carbonitrile; |
| 126 | (1S,2S)-2-((7-(8-ethynyl-7-fluoro-3-hydroxynaphthalen-1-yl)-8-fluoro-2-(((2R,7aS)-2-fluorotetrahydro- |
| | 1H-pyrrolizin-7a(5H)-yl)methoxy)pyrido[4,3-d]pyrimidin-4-yl)(methyl)amino)cyclopropane-1-carbonitrile |
| | 2,2,2-trifluoroacetic acid; |
| 127 | (1S,2S)-2-((7-(8-ethynyl-7-fluoro-3-hydroxynaphthalen-1-yl)-8-fluoro-2-(((2R,7aS)-2-fluorotetrahydro- |
| | 1H-pyrrolizin-7a(5H)-yl)methoxy)pyrido[4,3-d]pyrimidin-4-yl)(methyl)amino)cyclopropane-1-carbonitrile; |
| 128 | (1R,2R)-2-((7-(8-ethynyl-7-fluoro-3-hydroxynaphthalen-1-yl)-8-fluoro-2-(((2R,7aS)-2-fluorotetrahydro- |
| | 1H-pyrrolizin-7a(5H)-yl)methoxy)pyrido[4,3-d]pyrimidin-4-yl)(methyl)amino)cyclopropane-1-carbonitrile |
| | 2,2,2-trifluoroacetic acid; |
| 129 | (1R,2R)-2-((7-(8-ethynyl-7-fluoro-3-hydroxynaphthalen-1-yl)-8-fluoro-2-(((2R,7aS)-2-fluorotetrahydro- |
| | 1H-pyrrolizin-7a(5H)-yl)methoxy)pyrido[4,3-d]pyrimidin-4-yl)(methyl)amino)cyclopropane-1-carbonitrile; |
| 130 | 2-((7-(8-ethynyl-7-fluoro-3-hydroxynaphthalen-1-yl)-8-fluoro-2-(((2R,7aS)-2-fluorotetrahydro-1H- |
| | pyrrolizin-7a(5H)-yl)methoxy)pyrido[4,3-d]pyrimidin-4-yl)(methyl)amino)cyclopropane-1-carbonitrile; |
| 131 | 4-(4-(cyclopropyl(ethyl)amino)-8-fluoro-2-(((2R,7aS)-2-fluorotetrahydro-1H-pyrrolizin-7a(5H)- |
| | yl)methoxy)pyrido[4,3-d]pyrimidin-7-yl)-5-ethynyl-6-fluoronaphthalen-2-ol; |
| 132 | 4-(4-(dicyclopropylamino)-8-fluoro-2-(((2R,7aS)-2-fluorotetrahydro-1H-pyrrolizin-7a(5H)- |
| | yl)methoxy)pyrido[4,3-d]pyrimidin-7-yl)-5-ethynyl-6-fluoronaphthalen-2-ol; |
| 133 | 5-ethynyl-6-fluoro-4-(8-fluoro-2-(((2R,7aS)-2-fluorotetrahydro-1H-pyrrolizin-7a(5H)-yl)methoxy)-4- |
| | (methyl(2-methylcyclopropyl)amino)pyrido[4,3-d]pyrimidin-7-yl)naphthalen-2-ol; |
| 134 | 5-ethynyl-6-fluoro-4-(8-fluoro-2-(((2R,7aS)-2-fluorotetrahydro-1H-pyrrolizin-7a(5H)-yl)methoxy)-4- |
| | (((1S,2S)-2-hydroxycyclopropyl)(methyl)amino)pyrido[4,3-d]pyrimidin-7-yl)naphthalen-2-ol; |
| 135 | 4-(4-(cyclopropyl(methyl)amino)-8-fluoro-2-(((2R,7aS)-2-fluorotetrahydro-1H-pyrrolizin-7a(5H)-yl) |
| | methoxy)pyrido[4,3-d]pyrimidin-7-yl)-5-ethynyl-6,7-difluoronaphthalen-2-ol; |
| 136 | 5-ethynyl-6-fluoro-4-(8-fluoro-2-(((2R,7aS)-2-fluorotetrahydro-1H-pyrrolizin-7a(5H)-yl)methoxy)-4- |
| | ((2-hydroxycyclopropyl)(methyl)amino)pyrido[4,3-d]pyrimidin-7-yl)naphthalen-2-ol; |
| 137 | 7-(4-ethynyl-1H-indol-3-yl)-8-fluoro-N-((1S,2S)-2-fluorocyclopropyl)-2-(((2R,7aS)-2-fluorotetrahydro- |
| | 1H-pyrrolizin-7a(5H)-yl)methoxy)-N-methylpyrido[4,3-d]pyrimidin-4-amine; |
| 138 | 7-(4-ethynyl-1H-indol-3-yl)-8-fluoro-N-((1S,2S)-2-fluorocyclopropyl)-N-methyl-2-((1- |
| | (morpholinomethyl)cyclopropyl)methoxy)pyrido[4,3-d]pyrimidin-4-amine; |
| 139 | 7-(4-ethyl-1H-indol-3-yl)-8-fluoro-N-((1S,2S)-2-fluorocyclopropyl)-N-methyl-2-((1- |
| | (morpholinomethyl)cyclopropyl)methoxy)pyrido[4,3-d]pyrimidin-4-amine; |
| 140 | 7-(4-ethynyl-5-fluoro-1H-indol-3-yl)-8-fluoro-N-((1S,2S)-2-fluorocyclopropyl)-2-(((2R,7aS)-2- |
| | fluorotetrahydro-1H-pyrrolizin-7a(5H)-yl)methoxy)-N-methylpyrido[4,3-d]pyrimidin-4-amine; |
| 141 | 7-(4-ethyl-5-fluoro-1H-indol-3-yl)-8-fluoro-N-((1S,2S)-2-fluorocyclopropyl)-N-methyl-2-((1- |
| | (morpholinomethyl)cyclopropyl)methoxy)pyrido[4,3-d]pyrimidin-4-amine; |
| 142 | 5-ethynyl-6-fluoro-4-(8-fluoro-4-(((1S,2S)-2-fluorocyclopropyl)(methyl)amino)-2-(((2R,7aS)-2- |
| | (methylthio)tetrahydro-1H-pyrrolizin-7a(5H)-yl)methoxy)pyrido[4,3-d]pyrimidin-7-yl)naphthalen-2-ol; |
| 143 | 5-ethynyl-6-fluoro-4-(8-fluoro-2-(((2R,7aS)-2-fluoro-2-methyltetrahydro-1H-pyrrolizin-7a(5H)- |
| | yl)methoxy)-4-(((1S,2S)-2-fluorocyclopropyl)(methyl)amino)pyrido[4,3-d]pyrimidin-7-yl)naphthalen-2-ol; |
| 144 | 5-ethynyl-6,7-difluoro-4-(8-fluoro-4-(((1S,2S)-2-fluorocyclopropyl)(methyl)amino)-2-(((2R,7aS)-2- |
| | fluorotetrahydro-1H-pyrrolizin-7a(5H)-yl)methoxy)pyrido[4,3-d]pyrimidin-7-yl)naphthalen-2-ol; |
| 145 | 5-ethynyl-6-fluoro-4-(8-fluoro-2-(((2R,7aS)-2-fluorotetrahydro-1H-pyrrolizin-7a(5H)-yl)methoxy)-4- |
| | ((2-hydroxy-2-methylcyclopropyl)(methyl)amino)pyrido[4,3-d]pyrimidin-7-yl)naphthalen-2-ol; |
| 146 | 5-ethynyl-6-fluoro-4-(8-fluoro-4-(((1S,2S)-2-fluorocyclopropyl)(methyl-d3)amino)-2-(((2R,7aS)-2- |
| | fluorotetrahydro-1H-pyrrolizin-7a(5H)-yl)methoxy)pyrido[4,3-d]pyrimidin-7-yl)naphthalen-2-ol; |
| 147 | 5-ethynyl-6-fluoro-4-(8-fluoro-4-(((1S,2S)-2-fluorocyclopropyl)(methyl)amino)-2-(((2R,7aS)-2- |
| | fluorotetrahydro-1H-pyrrolizin-7a(5H)-yl)methoxy-d2)pyrido[4,3-d]pyrimidin-7-yl)naphthalen-2-ol; |
| 148 | 5-ethynyl-6-fluoro-4-(8-fluoro-4-(((1S,2S)-2-fluorocyclopropyl)(methyl-d3)amino)-2-(((2R,7aS)-2- |
| | fluorotetrahydro-1H-pyrrolizin-7a(5H)-yl)methoxy-d2)pyrido[4,3-d]pyrimidin-7-yl)naphthalen-2-ol; |
| 149 | 5-ethynyl-6-fluoro-4-(8-fluoro-4-(((1S,2S)-2-fluorocyclopropyl)(methyl-d3)amino)-2-(((2R,7aS)-2- |
| | fluorotetrahydro-1H-pyrrolizin-7a(5H)-yl-5,5-d2)methoxy-d2)pyrido[4,3-d]pyrimidin-7-yl)naphthalen-2-ol; |
| 150 | 5-ethynyl-6-fluoro-4-(8-fluoro-4-(((1S,2S)-2-fluorocyclopropyl)(methyl)amino)-2-(((2R,7aS)-2- |
| | fluorotetrahydro-1H-pyrrolizin-7a(5H)-yl-5,5-d2)methoxy-d2)pyrido[4,3-d]pyrimidin-7-yl)naphthalen-2-ol; |
| 151 | 5-ethynyl-6-fluoro-4-(8-fluoro-4-(((1S,2S)-2-fluorocyclopropyl)(methyl)amino)-2-(((2R,7aS)-2- |
| | fluorotetrahydro-1H-pyrrolizin-7a(5H)-yl-5,5-d2)methoxy)pyrido[4,3-d]pyrimidin-7-yl)naphthalen-2-ol; |
| 152 | 5-ethynyl-6-fluoro-4-(8-fluoro-4-(((1S,2S)-2-fluorocyclopropyl)(methyl-d3)amino)-2-(((2R,7aS)-2- |
| | fluorotetrahydro-1H-pyrrolizin-7a(5H)-yl-5,5-d2)methoxy)pyrido[4,3-d]pyrimidin-7-yl)naphthalen-2-ol; |
| 153 | 5-ethynyl-6-fluoro-4-(8-fluoro-4-(((1S,2S)-2-fluorocyclopropyl)(methyl-d3)amino)-2-(((2R,7aS)-2- |
| | fluorotetrahydro-1H-pyrrolizin-7a(5H)-yl-2,5,5-d3)methoxy-d2)pyrido[4,3-d]pyrimidin-7-yl)naphthalen-2-ol; |
| 154 | 5-ethynyl-6-fluoro-4-(8-fluoro-4-(((1S,2S)-2-fluorocyclopropyl)(methyl)amino)-2-(((2R,7aS)-2- |
| | fluorotetrahydro-1H-pyrrolizin-7a(5H)-yl-2,5,5-d3)methoxy-d2)pyrido[4,3-d]pyrimidin-7-yl)naphthalen-2-ol; |
| 155 | 5-ethynyl-6-fluoro-4-(8-fluoro-4-(((1S,2S)-2-fluorocyclopropyl)(methyl-d3)amino)-2-(((2R,7aS)-2- |
| | fluorotetrahydro-1H-pyrrolizin-7a(5H)-yl-2-d)methoxy)pyrido[4,3-d]pyrimidin-7-yl)naphthalen-2-ol; |
| 156 | 5-ethynyl-6-fluoro-4-(8-fluoro-4-(((1S,2S)-2-fluorocyclopropyl)(methyl)amino)-2-(((2R,7aS)-2- |
| | fluorotetrahydro-1H-pyrrolizin-7a(5H)-yl-2-d)methoxy)pyrido[4,3-d]pyrimidin-7-yl)naphthalen-2-ol; |
| 157 | 4-(4-((2-(difluoromethyl)cyclopropyl)(methyl)amino)-8-fluoro-2-(((2R,7aS)-2-fluorotetrahydro-1H- |
| | pyrrolizin-7a(5H)-yl)methoxy)pyrido[4,3-d]pyrimidin-7-yl)-5-ethynyl-6-fluoronaphthalen-2-ol; |
| 158 | 5-ethynyl-6-fluoro-4-(8-fluoro-4-(((1S,2S)-2-(fluoromethyl)cyclopropyl)(methyl)amino)-2-(((2R,7aS)-2- |
| | fluorotetrahydro-1H-pyrrolizin-7a(5H)-yl)methoxy)pyrido[4,3-d]pyrimidin-7-yl)naphthalen-2-ol; |
| 159 | 1-(2-((7-(8-ethynyl-7-fluoro-3-hydroxynaphthalen-1-yl)-8-fluoro-2-(((2R,7aS)-2-fluorotetrahydro- |
| | 1H-pyrrolizin-7a(5H)-yl)methoxy)pyrido[4,3-d]pyrimidin-4-yl)(methyl)amino)cyclopropyl)ethan-1-one; |
| 160 | 2-(2-((7-(8-ethynyl-7-fluoro-3-hydroxynaphthalen-1-yl)-8-fluoro-2-(((2R,7aS)-2-fluorotetrahydro-1H- |
| | pyrrolizin-7a(5H)-yl)methoxy)pyrido[4,3-d]pyrimidin-4-yl)(methyl)amino)cyclopropyl)acetonitrile; |
| 161 | 5-ethynyl-6-fluoro-4-(8-fluoro-2-(((2R,7aS)-2-fluorotetrahydro-1H-pyrrolizin-7a(5H)-yl)methoxy)-4- |
| | (methyl((1S,2S)-2-(methylamino)cyclopropyl)amino)pyrido[4,3-d]pyrimidin-7-yl)naphthalen-2-ol; |
| 162 | 5-ethynyl-6-fluoro-4-(8-fluoro-2-(((2R,7aS)-2-fluorotetrahydro-1H-pyrrolizin-7a(5H)-yl)methoxy)-4- |
| | (methyl((1S,2S)-2-(methylthio)cyclopropyl)amino)pyrido[4,3-d]pyrimidin-7-yl)naphthalen-2-ol; |
| 163 | 5-ethynyl-6-fluoro-4-(8-fluoro-4-(((1S,2S)-2-fluorocyclopropyl)(methyl)amino)-2-(((2R,7aS)-2- |
| | fluorotetrahydro-1H-pyrrolizin-7a(5H)-yl)methoxy)-5-methoxypyrido[4,3-d]pyrimidin-7-yl)naphthalen-2-ol; |
| 164 | 4-(5-cyclopropoxy-8-fluoro-4-(((1S,2S)-2-fluorocyclopropyl)(methyl)amino)-2-(((2R,7aS)-2-fluorotetrahydro- |
| | 1H-pyrrolizin-7a(5H)-yl)methoxy)pyrido[4,3-d]pyrimidin-7-yl)-5-ethynyl-6-fluoronaphthalen-2-ol; |
| 165 | 5-ethynyl-6-fluoro-4-(8-fluoro-4-(((1S,2S)-2-fluorocyclopropyl)(methyl)amino)-2-(((2R,7aS)-2-fluorotetrahydro- |
| | 1H-pyrrolizin-7a(5H)-yl)methoxy)-5-(methylthio)pyrido[4,3-d]pyrimidin-7-yl)naphthalen-2-ol; |
| 166 | 5-ethynyl-6-fluoro-4-(8-fluoro-4-(((1S,2S)-2-fluorocyclopropyl)(methyl)amino)-2-(((2R,7aS)-2-fluorotetrahydro- |
| | 1H-pyrrolizin-7a(5H)-yl)methoxy)-5-(methylamino)pyrido[4,3-d]pyrimidin-7-yl)naphthalen-2-ol; |
| 167 | 5-ethynyl-6-fluoro-4-(8-fluoro-4-(((1S,2S)-2-fluorocyclopropyl)(methyl)amino)-2-(((2R,7aS)-2- |
| | fluorotetrahydro-1H-pyrrolizin-7a(5H)-yl)methoxy)-5-(trifluoromethoxy)pyrido[4,3-d]pyrimidin-7- |
| | yl)naphthalen-2-ol; |
| 168 | 4-(5-cyclopropyl-8-fluoro-4-(((1S,2S)-2-fluorocyclopropyl)(methyl)amino)-2-(((2R,7aS)-2-fluorotetrahydro- |
| | 1H-pyrrolizin-7a(5H)-yl)methoxy)pyrido[4,3-d]pyrimidin-7-yl)-5-ethynyl-6-fluoronaphthalen-2-ol; |
| 169 | 5-ethynyl-6-fluoro-4-(8-fluoro-4-(((1S,2S)-2-fluorocyclopropyl)(methyl)amino)-2-(((2R,7aS)-2- |
| | fluorotetrahydro-1H-pyrrolizin-7a(5H)-yl)methoxy)-5-methylpyrido[4,3-d]pyrimidin-7-yl)naphthalen-2-ol |
| 170 | 7-(8-ethynyl-7-fluoro-3-hydroxynaphthalen-1-yl)-8-fluoro-4-(((1S,2S)-2-fluorocyclopropyl)(methyl)amino)- |
| | 2-(((2R,7aS)-2-fluorotetrahydro-1H-pyrrolizin-7a(5H)-yl)methoxy)pyrido[4,3-d]pyrimidine-5-carbonitrile; |
| 171 | 4-(5-(difluoromethyl)-8-fluoro-4-(((1S,2S)-2-fluorocyclopropyl)(methyl)amino)-2-(((2R,7aS)-2-fluorotetrahydro- |
| | 1H-pyrrolizin-7a(5H)-yl)methoxy)pyrido[4,3-d]pyrimidin-7-yl)-5-ethynyl-6-fluoronaphthalen-2-ol; |
| 172 | 5-ethynyl-6-fluoro-4-(8-fluoro-4-(((1S,2S)-2-fluorocyclopropyl)(methyl)amino)-5-(fluoromethyl)-2- |
| | (((2R,7aS)-2-fluorotetrahydro-1H-pyrrolizin-7a(5H)-yl)methoxy)pyrido[4,3-d]pyrimidin-7-yl)naphthalen-2-ol; |
| 173 | 5-ethynyl-6-fluoro-4-(8-fluoro-4-(((1S,2S)-2-fluorocyclopropyl)(methyl)amino)-2-(((2R,7aS)-2-fluorotetrahydro- |
| | 1H-pyrrolizin-7a(5H)-yl)methoxy)-5-(hydroxymethyl)pyrido[4,3-d]pyrimidin-7-yl)naphthalen-2-ol; |
| 174 | 7-(8-ethynyl-7-fluoro-3-hydroxynaphthalen-1-yl)-8-fluoro-4-(((1S,2S)-2-fluorocyclopropyl)(methyl)amino)- |
| | 2-(((2R,7aS)-2-fluorotetrahydro-1H-pyrrolizin-7a(5H)-yl)methoxy)pyrido[4,3-d]pyrimidine-5-carboxylic acid; |
| 175 | 7-(8-ethynyl-7-fluoro-3-hydroxynaphthalen-1-yl)-8-fluoro-4-(((1S,2S)-2-fluorocyclopropyl)(methyl)amino)- |
| | 2-(((2R,7aS)-2-fluorotetrahydro-1H-pyrrolizin-7a(5H)-yl)methoxy)pyrido[4,3-d]pyrimidine-5-carboxamide; |
| 176 | methyl |
| | 7-(8-ethynyl-7-fluoro-3-hydroxynaphthalen-1-yl)-8-fluoro-4-(((1S,2S)-2- |
| | fluorocyclopropyl)(methyl)amino)-2-(((2R,7aS)-2-fluorotetrahydro-1H-pyrrolizin-7a(5H)- |
| | yl)methoxy)pyrido[4,3-d]pyrimidine-5-carboxylate; |
| 177 | 4-(4-(((2R,3R)-2,3-difluorocyclopropyl)(methyl)amino)-8-fluoro-2-(((2R,7aS)-2-fluorotetrahydro- |
| | 1H-pyrrolizin-7a(5H)-yl)methoxy)pyrido[4,3-d]pyrimidin-7-yl)-5-ethynyl-6-fluoronaphthalen-2-ol; |
| 178 | 4-(4-(((1s,2R,3S)-2,3-difluorocyclopropyl)(methyl)amino)-8-fluoro-2-(((2R,7aS)-2-fluorotetrahydro- |
| | 1H-pyrrolizin-7a(5H)-yl)methoxy)pyrido[4,3-d]pyrimidin-7-yl)-5-ethynyl-6-fluoronaphthalen-2-ol; |
| 179 | 5-ethynyl-6-fluoro-4-(8-fluoro-4-(((1S,2S,3R)-2-fluoro-3-methylcyclopropyl)(methyl)amino)-2-(((2R,7aS)- |
| | 2-fluorotetrahydro-1H-pyrrolizin-7a(5H)-yl)methoxy)pyrido[4,3-d]pyrimidin-7-yl)naphthalen-2-ol; |
| 180 | 5-ethynyl-6-fluoro-4-(8-fluoro-4-(((1S,2S,3S)-2-fluoro-3-methylcyclopropyl)(methyl)amino)-2-(((2R,7aS)- |
| | 2-fluorotetrahydro-1H-pyrrolizin-7a(5H)-yl)methoxy)pyrido[4,3-d]pyrimidin-7-yl)naphthalen-2-ol; |
| 181 | 5,6-difluoro-4-(8-fluoro-4-(((1S,2S)-2-fluorocyclopropyl)(methyl)amino)-2-(((2R,7aS)-2-fluorotetrahydro- |
| | 1H-pyrrolizin-7a(5H)-yl)methoxy)pyrido[4,3-d]pyrimidin-7-yl)naphthalen-2-ol; |
| 182 | 4-(4-(ethyl(2-fluorocyclopropyl)amino)-8-fluoro-2-(((2R,7aS)-2-fluoro-2-methyltetrahydro-1H-pyrrolizin- |
| | 7a(5H)-yl)methoxy)pyrido[4,3-d]pyrimidin-7-yl)-5-ethynyl-6,7-difluoronaphthalen-2-ol; |
| 183 | 4-(4-(ethyl(2-fluorocyclopropyl)amino)-8-fluoro-2-(((2R,7aS)-2-fluorotetrahydro-1H-pyrrolizin-7a(5H)- |
| | yl)methoxy)pyrido[4,3-d]pyrimidin-7-yl)-5-ethynyl-6,7-difluoronaphthalen-2-ol; |
| 184 | 4-(4-(ethyl(2-fluorocyclopropyl)amino)-8-fluoro-2-(((2R,7aS)-2-fluorotetrahydro-1H-pyrrolizin-7a(5H)- |
| | yl)methoxy-d2)pyrido[4,3-d]pyrimidin-7-yl)-5-ethynyl-6,7-difluoronaphthalen-2-ol; |
| 185 | 4-(4-(ethyl(2-fluorocyclopropyl)amino)-8-fluoro-2-(((2R,7aS)-2-(methylthio)tetrahydro-1H-pyrrolizin- |
| | 7a(5H)-yl)methoxy)pyrido[4,3-d]pyrimidin-7-yl)-5-ethynyl-6,7-difluoronaphthalen-2-ol; |
| 186 | 4-(4-(ethyl(2-fluorocyclopropyl)amino)-8-fluoro-2-(((2R,7aS)-2-fluorotetrahydro-1H-pyrrolizin-7a(5H)- |
| | yl-5,5-d2)methoxy-d2)pyrido[4,3-d]pyrimidin-7-yl)-5-ethynyl-6,7-difluoronaphthalen-2-ol; |
| 187 | 5-ethynyl-6,7-difluoro-4-(8-fluoro-2-(((2R,7aS)-2-fluoro-2-methyltetrahydro-1H-pyrrolizin-7a(5H)- |
| | yl)methoxy)-4-((2-fluorocyclopropyl)(methyl-d3)amino)pyrido[4,3-d]pyrimidin-7-yl)naphthalen-2-ol; |
| 188 | 5-ethynyl-6,7-difluoro-4-(8-fluoro-4-((2-fluorocyclopropyl)(methyl-d3)amino)-2-(((2R,7aS)-2- |
| | fluorotetrahydro-1H-pyrrolizin-7a(5H)-yl)methoxy)pyrido[4,3-d]pyrimidin-7-yl)naphthalen-2-ol; |
| 189 | 5-ethynyl-6,7-difluoro-4-(8-fluoro-4-((2-fluorocyclopropyl)(methyl-d3)amino)-2-(((2R,7aS)-2- |
| | fluorotetrahydro-1H-pyrrolizin-7a(5H)-yl)methoxy-d2)pyrido[4,3-d]pyrimidin-7-yl)naphthalen-2-ol; |
| 190 | 5-ethynyl-6,7-difluoro-4-(8-fluoro-4-((2-fluorocyclopropyl)(methyl-d3)amino)-2-(((2R,7aS)-2- |
| | (methylthio)tetrahydro-1H-pyrrolizin-7a(5H)-yl)methoxy)pyrido[4,3-d]pyrimidin-7-yl)naphthalen-2-ol; |
| 191 | 5-ethynyl-6,7-difluoro-4-(8-fluoro-4-((2-fluorocyclopropyl)(methyl-d3)amino)-2-(((2R,7aS)-2- |
| | fluorotetrahydro-1H-pyrrolizin-7a(5H)-yl-5,5-d2)methoxy-d2)pyrido[4,3-d]pyrimidin-7-yl)naphthalen-2-ol; |
| 192 | 5-ethynyl-6,7-difluoro-4-(8-fluoro-2-(((2R,7aS)-2-fluoro-2-methyltetrahydro-1H-pyrrolizin-7a(5H)- |
| | yl)methoxy)-4-((2-fluorocyclopropyl)(methyl)amino)pyrido[4,3-d]pyrimidin-7-yl)naphthalen-2-ol; |
| 193 | 5-ethynyl-6,7-difluoro-4-(8-fluoro-4-((2-fluorocyclopropyl)(methyl)amino)-2-(((2R,7aS)-2- |
| | fluorotetrahydro-1H-pyrrolizin-7a(5H)-yl)methoxy)pyrido[4,3-d]pyrimidin-7-yl)naphthalen-2-ol; |
| 194 | 5-ethynyl-6,7-difluoro-4-(8-fluoro-4-((2-fluorocyclopropyl)(methyl)amino)-2-(((2R,7aS)-2- |
| | fluorotetrahydro-1H-pyrrolizin-7a(5H)-yl)methoxy-d2)pyrido[4,3-d]pyrimidin-7-yl)naphthalen-2-ol; |
| 195 | 5-ethynyl-6,7-difluoro-4-(8-fluoro-4-((2-fluorocyclopropyl)(methyl)amino)-2-(((2R,7aS)-2- |
| | (methylthio)tetrahydro-1H-pyrrolizin-7a(5H)-yl)methoxy)pyrido[4,3-d]pyrimidin-7-yl)naphthalen-2-ol; |
| 196 | 5-ethynyl-6,7-difluoro-4-(8-fluoro-4-((2-fluorocyclopropyl)(methyl)amino)-2-(((2R,7aS)-2- |
| | fluorotetrahydro-1H-pyrrolizin-7a(5H)-yl-5,5-d2)methoxy-d2)pyrido[4,3-d]pyrimidin-7-yl)naphthalen-2-ol; |
| 197 | 5-ethynyl-6,7-difluoro-4-(8-fluoro-4-(((1S,2S)-2-fluorocyclopropyl)(methyl)amino)-2-(((2R,7aS)-2- |
| | fluorotetrahydro-1H-pyrrolizin-7a(5H)-yl-2-d)methoxy)pyrido[4,3-d]pyrimidin-7-yl)naphthalen-2-ol; |
| 198 | 5-ethynyl-6,7-difluoro-4-(8-fluoro-2-(((2R,7aS)-2-fluoro-2-methyltetrahydro-1H-pyrrolizin-7a(5H)-yl)methoxy)- |
| | 4-(((1S,2S)-2-fluorocyclopropyl)(methyl)amino)pyrido[4,3-d]pyrimidin-7-yl)naphthalen-2-ol; |
| 199 | 5-ethynyl-6,7-difluoro-4-(8-fluoro-4-(((1S,2S)-2-fluorocyclopropyl)(methyl)amino)-2-(((2R,7aS)-2- |
| | methoxytetrahydro-1H-pyrrolizin-7a(5H)-yl)methoxy)pyrido[4,3-d]pyrimidin-7-yl)naphthalen-2-ol; |
| 200 | 5-ethynyl-6,7-difluoro-4-(8-fluoro-4-(((1S,2S)-2-fluorocyclopropyl)(methyl)amino)-2-(((2R,7aS)-2- |
| | fluorotetrahydro-1H-pyrrolizin-7a(5H)-yl)methoxy-d2)pyrido[4,3-d]pyrimidin-7-yl)naphthalen-2-ol; |
| 201 | 5-ethynyl-6,7-difluoro-4-(8-fluoro-4-(((1S,2S)-2-fluorocyclopropyl)(methyl)amino)-2-(((2R,7aS)-2- |
| | (methylthio)tetrahydro-1H-pyrrolizin-7a(5H)-yl)methoxy)pyrido[4,3-d]pyrimidin-7-yl)naphthalen-2-ol |
| 202 | 5-ethynyl-6,7-difluoro-4-(8-fluoro-4-(((1S,2S)-2-fluorocyclopropyl)(methyl)amino)-2-(((2R,7aS)-2- |
| | fluorotetrahydro-1H-pyrrolizin-7a(5H)-yl-2-d)methoxy-d2)pyrido[4,3-d]pyrimidin-7-yl)naphthalen-2-ol; |
| 203 | 5-ethynyl-6,7-difluoro-4-(8-fluoro-4-(((1S,2S)-2-fluorocyclopropyl)(methyl)amino)-2-(((2R,7aS)-2- |
| | fluorotetrahydro-1H-pyrrolizin-7a(5H)-yl-2,5,5-d3)methoxy-d2)pyrido[4,3-d]pyrimidin-7-yl)naphthalen-2-ol; |
| 204 | 5-ethynyl-6,7-difluoro-4-(8-fluoro-4-(((1S,2S)-2-fluorocyclopropyl)(methyl)amino)-2-(((2R,7aS)-2- |
| | fluorotetrahydro-1H-pyrrolizin-7a(5H)-yl-5,5-d2)methoxy-d2)pyrido[4,3-d]pyrimidin-7-yl)naphthalen-2-ol; |
| 205 | 4-(4-(ethyl((1S,2S)-2-fluorocyclopropyl)amino)-8-fluoro-2-(((2R,7aS)-2-fluorotetrahydro-1H-pyrrolizin- |
| | 7a(5H)-yl-2-d)methoxy)pyrido[4,3-d]pyrimidin-7-yl)-5-ethynyl-6,7-difluoronaphthalen-2-ol; |
| 206 | 4-(4-(ethyl((1S,2S)-2-fluorocyclopropyl)amino)-8-fluoro-2-(((2R,7aS)-2-fluoro-2-methyltetrahydro-1H- |
| | pyrrolizin-7a(5H)-yl)methoxy)pyrido[4,3-d]pyrimidin-7-yl)-5-ethynyl-6,7-difluoronaphthalen-2-ol; |
| 207 | 4-(4-(ethyl((1S,2S)-2-fluorocyclopropyl)amino)-8-fluoro-2-(((2R,7aS)-2-methoxytetrahydro-1H- |
| | pyrrolizin-7a(5H)-yl)methoxy)pyrido[4,3-d]pyrimidin-7-yl)-5-ethynyl-6,7-difluoronaphthalen-2-ol; |
| 208 | 4-(4-(ethyl((1S,2S)-2-fluorocyclopropyl)amino)-8-fluoro-2-(((2R,7aS)-2-fluorotetrahydro-1H-pyrrolizin- |
| | 7a(5H)-yl)methoxy-d2)pyrido[4,3-d]pyrimidin-7-yl)-5-ethynyl-6,7-difluoronaphthalen-2-ol; |
| 209 | 4-(4-(ethyl((1S,2S)-2-fluorocyclopropyl)amino)-8-fluoro-2-(((2R,7aS)-2-(methylthio)tetrahydro-1H- |
| | pyrrolizin-7a(5H)-yl)methoxy)pyrido[4,3-d]pyrimidin-7-yl)-5-ethynyl-6,7-difluoronaphthalen-2-ol; |
| 210 | 4-(4-(ethyl((1S,2S)-2-fluorocyclopropyl)amino)-8-fluoro-2-(((2R,7aS)-2-fluorotetrahydro-1H-pyrrolizin- |
| | 7a(5H)-yl-2-d)methoxy-d2)pyrido[4,3-d]pyrimidin-7-yl)-5-ethynyl-6,7-difluoronaphthalen-2-ol; |
| 211 | 4-(4-(ethyl((1S,2S)-2-fluorocyclopropyl)amino)-8-fluoro-2-(((2R,7aS)-2-fluorotetrahydro-1H-pyrrolizin- |
| | 7a(5H)-yl-2,5,5-d3)methoxy-d2)pyrido[4,3-d]pyrimidin-7-yl)-5-ethynyl-6,7-difluoronaphthalen-2-ol; |
| 212 | 4-(4-(ethyl((1S,2S)-2-fluorocyclopropyl)amino)-8-fluoro-2-(((2R,7aS)-2-fluorotetrahydro-1H-pyrrolizin- |
| | 7a(5H)-yl-5,5-d2)methoxy-d2)pyrido[4,3-d]pyrimidin-7-yl)-5-ethynyl-6,7-difluoronaphthalen-2-ol; |
| 213 | 4-(4-(ethyl((1S,2S)-2-fluorocyclopropyl)amino)-8-fluoro-2-(((2R,7aS)-2-fluorotetrahydro-1H-pyrrolizin- |
| | 7a(5H)-yl)methoxy)pyrido[4,3-d]pyrimidin-7-yl)-5-ethynyl-6,7-difluoronaphthalen-2-ol; |
| 214 | 5-ethynyl-6,7-difluoro-4-(8-fluoro-4-(((1S,2S)-2-fluorocyclopropyl)(methyl-d3)amino)-2-(((2R,7aS)- |
| | 2-fluorotetrahydro-1H-pyrrolizin-7a(5H)-yl-2-d)methoxy)pyrido[4,3-d]pyrimidin-7-yl)naphthalen-2-ol; |
| 215 | 5-ethynyl-6,7-difluoro-4-(8-fluoro-2-(((2R,7aS)-2-fluoro-2-methyltetrahydro-1H-pyrrolizin-7a(5H)-yl)methoxy)- |
| | 4-(((1S,2S)-2-fluorocyclopropyl)(methyl-d3)amino)pyrido[4,3-d]pyrimidin-7-yl)naphthalen-2-ol; |
| 216 | 5-ethynyl-6,7-difluoro-4-(8-fluoro-4-(((1S,2S)-2-fluorocyclopropyl)(methyl-d3)amino)-2-(((2R,7aS)- |
| | 2-methoxytetrahydro-1H-pyrrolizin-7a(5H)-yl)methoxy)pyrido[4,3-d]pyrimidin-7-yl)naphthalen-2-ol; |
| 217 | 5-ethynyl-6,7-difluoro-4-(8-fluoro-4-(((1S,2S)-2-fluorocyclopropyl)(methyl-d3)amino)-2-(((2R,7aS)- |
| | 2-fluorotetrahydro-1H-pyrrolizin-7a(5H)-yl)methoxy-d2)pyrido[4,3-d]pyrimidin-7-yl)naphthalen-2-ol; |
| 218 | 5-ethynyl-6,7-difluoro-4-(8-fluoro-4-(((1S,2S)-2-fluorocyclopropyl)(methyl-d3)amino)-2-(((2R,7aS)- |
| | 2-(methylthio)tetrahydro-1H-pyrrolizin-7a(5H)-yl)methoxy)pyrido[4,3-d]pyrimidin-7-yl)naphthalen-2-ol; |
| 219 | 5-ethynyl-6,7-difluoro-4-(8-fluoro-4-(((1S,2S)-2-fluorocyclopropyl)(methyl-d3)amino)-2-(((2R,7aS)- |
| | 2-fluorotetrahydro-1H-pyrrolizin-7a(5H)-yl-2-d)methoxy-d2)pyrido[4,3-d]pyrimidin-7-yl)naphthalen-2-ol; |
| 220 | 5-ethynyl-6,7-difluoro-4-(8-fluoro-4-(((1S,2S)-2-fluorocyclopropyl)(methyl-d3)amino)-2-(((2R,7aS)- |
| | 2-fluorotetrahydro-1H-pyrrolizin-7a(5H)-yl-2,5,5-d3)methoxy-d2)pyrido[4,3-d]pyrimidin-7-yl)naphthalen-2-ol; |
| 221 | 5-ethynyl-6,7-difluoro-4-(8-fluoro-4-(((1S,2S)-2-fluorocyclopropyl)(methyl-d3)amino)-2-(((2R,7aS)- |
| | 2-fluorotetrahydro-1H-pyrrolizin-7a(5H)-yl-5,5-d2)methoxy-d2)pyrido[4,3-d]pyrimidin-7-yl)naphthalen-2-ol; |
| 222 | 5-ethynyl-6,7-difluoro-4-(8-fluoro-4-(((1S,2S)-2-fluorocyclopropyl)(methyl-d3)amino)-2-(((2R,7aS)- |
| | 2-fluorotetrahydro-1H-pyrrolizin-7a(5H)-yl)methoxy)pyrido[4,3-d]pyrimidin-7-yl)naphthalen-2-ol; |
| 223 | 4-(4-(ethyl((1S,2S)-2-fluorocyclopropyl)amino)-8-fluoro-2-(((2R,7aS)-2-fluorotetrahydro-1H-pyrrolizin- |
| | 7a(5H)-yl-2-d)methoxy)pyrido[4,3-d]pyrimidin-7-yl)-5-ethynyl-6-fluoronaphthalen-2-ol; |
| 224 | 4-(8-fluoro-4-(((1S,2S)-2-fluorocyclopropyl)(methyl-d3)amino)-2-(((2R,7aS)-2-fluorotetrahydro-1H- |
| | pyrrolizin-7a(5H)-yl-2-d)methoxy)pyrido[4,3-d]pyrimidin-7-yl)-5-(methylthio)naphthalen-2-ol; |
| 225 | 4-(4-(ethyl((1S,2S)-2-fluorocyclopropyl)amino)-8-fluoro-2-(((2R,7aS)-2-fluorotetrahydro-1H-pyrrolizin- |
| | 7a(5H)-yl-2-d)methoxy)pyrido[4,3-d]pyrimidin-7-yl)-5-(methylthio)naphthalen-2-ol; |
| 226 | 4-(8-fluoro-4-(((1S,2S)-2-fluorocyclopropyl)(methyl)amino)-2-(((2R,7aS)-2-fluorotetrahydro-1H-pyrrolizin- |
| | 7a(5H)-yl-2-d)methoxy)pyrido[4,3-d]pyrimidin-7-yl)-5-(methylthio)naphthalen-2-ol; |
| 227 | 5-bromo-6-fluoro-4-(8-fluoro-4-(((1S,2S)-2-fluorocyclopropyl)(methyl-d3)amino)-2-(((2R,7aS)-2- |
| | fluorotetrahydro-1H-pyrrolizin-7a(5H)-yl-2-d)methoxy)pyrido[4,3-d]pyrimidin-7-yl)naphthalen-2-ol; |
| 228 | 5-bromo-4-(4-(ethyl((1S,2S)-2-fluorocyclopropyl)amino)-8-fluoro-2-(((2R,7aS)-2-fluorotetrahydro- |
| | 1H-pyrrolizin-7a(5H)-yl-2-d)methoxy)pyrido[4,3-d]pyrimidin-7-yl)-6-fluoronaphthalen-2-ol; |
| 229 | 5-bromo-6-fluoro-4-(8-fluoro-4-(((1S,2S)-2-fluorocyclopropyl)(methyl)amino)-2-(((2R,7aS)-2- |
| | fluorotetrahydro-1H-pyrrolizin-7a(5H)-yl-2-d)methoxy)pyrido[4,3-d]pyrimidin-7-yl)naphthalen-2-ol; |
| 230 | 5-ethyl-4-(8-fluoro-4-(((1S,2S)-2-fluorocyclopropyl)(methyl-d3)amino)-2-(((2R,7aS)-2-fluorotetrahydro- |
| | 1H-pyrrolizin-7a(5H)-yl-2-d)methoxy)pyrido[4,3-d]pyrimidin-7-yl)naphthalen-2-ol; |
| 231 | 5-ethyl-4-(4-(ethyl((1S,2S)-2-fluorocyclopropyl)amino)-8-fluoro-2-(((2R,7aS)-2-fluorotetrahydro-1H- |
| | pyrrolizin-7a(5H)-yl-2-d)methoxy)pyrido[4,3-d]pyrimidin-7-yl)naphthalen-2-ol; |
| 232 | 5-ethyl-4-(8-fluoro-4-(((1S,2S)-2-fluorocyclopropyl)(methyl)amino)-2-(((2R,7aS)-2-fluorotetrahydro- |
| | 1H-pyrrolizin-7a(5H)-yl-2-d)methoxy)pyrido[4,3-d]pyrimidin-7-yl)naphthalen-2-ol; |
| 233 | 5,6-difluoro-4-(8-fluoro-4-(((1S,2S)-2-fluorocyclopropyl)(methyl-d3)amino)-2-(((2R,7aS)-2- |
| | fluorotetrahydro-1H-pyrrolizin-7a(5H)-yl-2-d)methoxy)pyrido[4,3-d]pyrimidin-7-yl)naphthalen-2-ol; |
| 234 | 4-(4-(ethyl((1S,2S)-2-fluorocyclopropyl)amino)-8-fluoro-2-(((2R,7aS)-2-fluorotetrahydro-1H- |
| | pyrrolizin-7a(5H)-yl-2-d)methoxy)pyrido[4,3-d]pyrimidin-7-yl)-5,6-difluoronaphthalen-2-ol; |
| 235 | 5,6-difluoro-4-(8-fluoro-4-(((1S,2S)-2-fluorocyclopropyl)(methyl)amino)-2-(((2R,7aS)-2-fluorotetrahydro- |
| | 1H-pyrrolizin-7a(5H)-yl-2-d)methoxy)pyrido[4,3-d]pyrimidin-7-yl)naphthalen-2-ol; |
| 236 | 5-chloro-6-fluoro-4-(8-fluoro-4-(((1S,2S)-2-fluorocyclopropyl)(methyl-d3)amino)-2-(((2R,7aS)-2- |
| | fluorotetrahydro-1H-pyrrolizin-7a(5H)-yl-2-d)methoxy)pyrido[4,3-d]pyrimidin-7-yl)naphthalen-2-ol; |
| 237 | 5-chloro-4-(4-(ethyl((1S,2S)-2-fluorocyclopropyl)amino)-8-fluoro-2-(((2R,7aS)-2-fluorotetrahydro- |
| | 1H-pyrrolizin-7a(5H)-yl-2-d)methoxy)pyrido[4,3-d]pyrimidin-7-yl)-6-fluoronaphthalen-2-ol; |
| 238 | 5-chloro-6-fluoro-4-(8-fluoro-4-(((1S,2S)-2-fluorocyclopropyl)(methyl)amino)-2-(((2R,7aS)-2- |
| | fluorotetrahydro-1H-pyrrolizin-7a(5H)-yl-2-d)methoxy)pyrido[4,3-d]pyrimidin-7-yl)naphthalen-2-ol; |
| 239 | 5-ethyl-6-fluoro-4-(8-fluoro-4-(((1S,2S)-2-fluorocyclopropyl)(methyl-d3)amino)-2-(((2R,7aS)-2- |
| | fluorotetrahydro-1H-pyrrolizin-7a(5H)-yl-2-d)methoxy)pyrido[4,3-d]pyrimidin-7-yl)naphthalen-2-ol; |
| 240 | 5-ethyl-4-(4-(ethyl((1S,2S)-2-fluorocyclopropyl)amino)-8-fluoro-2-(((2R,7aS)-2-fluorotetrahydro- |
| | 1H-pyrrolizin-7a(5H)-yl-2-d)methoxy)pyrido[4,3-d]pyrimidin-7-yl)-6-fluoronaphthalen-2-ol; |
| 241 | 5-ethyl-6-fluoro-4-(8-fluoro-4-(((1S,2S)-2-fluorocyclopropyl)(methyl)amino)-2-(((2R,7aS)-2- |
| | fluorotetrahydro-1H-pyrrolizin-7a(5H)-yl-2-d)methoxy)pyrido[4,3-d]pyrimidin-7-yl)naphthalen-2-ol; |
| 242 | 5-ethynyl-4-(8-fluoro-4-(((1S,2S)-2-fluorocyclopropyl)(methyl-d3)amino)-2-(((2R,7aS)-2- |
| | fluorotetrahydro-1H-pyrrolizin-7a(5H)-yl-2-d)methoxy)pyrido[4,3-d]pyrimidin-7-yl)naphthalen-2-ol; |
| 243 | 4-(4-(ethyl((1S,2S)-2-fluorocyclopropyl)amino)-8-fluoro-2-(((2R,7aS)-2-fluorotetrahydro-1H- |
| | pyrrolizin-7a(5H)-yl-2-d)methoxy)pyrido[4,3-d]pyrimidin-7-yl)-5-ethynylnaphthalen-2-ol; |
| 244 | 5-ethynyl-4-(8-fluoro-4-(((1S,2S)-2-fluorocyclopropyl)(methyl)amino)-2-(((2R,7aS)-2-fluorotetrahydro- |
| | 1H-pyrrolizin-7a(5H)-yl-2-d)methoxy)pyrido[4,3-d]pyrimidin-7-yl)naphthalen-2-ol; |
| 245 | 5-ethynyl-6-fluoro-4-(8-fluoro-4-(((1S,2S)-2-fluorocyclopropyl)(methyl-d3)amino)-2-(((2R,7aS)-2- |
| | methoxytetrahydro-1H-pyrrolizin-7a(5H)-yl)methoxy)pyrido[4,3-d]pyrimidin-7-yl)naphthalen-2-ol; |
| 246 | 4-(4-(ethyl((1S,2S)-2-fluorocyclopropyl)amino)-8-fluoro-2-(((2R,7aS)-2-methoxytetrahydro-1H- |
| | pyrrolizin-7a(5H)-yl)methoxy)pyrido[4,3-d]pyrimidin-7-yl)-5-ethynyl-6-fluoronaphthalen-2-ol; |
| 247 | 4-(8-fluoro-4-(((1S,2S)-2-fluorocyclopropyl)(methyl-d3)amino)-2-(((2R,7aS)-2-methoxytetrahydro- |
| | 1H-pyrrolizin-7a(5H)-yl)methoxy)pyrido[4,3-d]pyrimidin-7-yl)-5-(methylthio)naphthalen-2-ol; |
| 248 | 4-(4-(ethyl((1S,2S)-2-fluorocyclopropyl)amino)-8-fluoro-2-(((2R,7aS)-2-methoxytetrahydro-1H- |
| | pyrrolizin-7a(5H)-yl)methoxy)pyrido[4,3-d]pyrimidin-7-yl)-5-(methylthio)naphthalen-2-ol; |
| 249 | 4-(8-fluoro-4-(((1S,2S)-2-fluorocyclopropyl)(methyl)amino)-2-(((2R,7aS)-2-methoxytetrahydro-1H- |
| | pyrrolizin-7a(5H)-yl)methoxy)pyrido[4,3-d]pyrimidin-7-yl)-5-(methylthio)naphthalen-2-ol; |
| 250 | 5-bromo-6-fluoro-4-(8-fluoro-4-(((1S,2S)-2-fluorocyclopropyl)(methyl-d3)amino)-2-(((2R,7aS)-2- |
| | methoxytetrahydro-1H-pyrrolizin-7a(5H)-yl)methoxy)pyrido[4,3-d]pyrimidin-7-yl)naphthalen-2-ol; |
| 251 | 5-bromo-4-(4-(ethyl((1S,2S)-2-fluorocyclopropyl)amino)-8-fluoro-2-(((2R,7aS)-2-methoxytetrahydro- |
| | 1H-pyrrolizin-7a(5H)-yl)methoxy)pyrido[4,3-d]pyrimidin-7-yl)-6-fluoronaphthalen-2-ol; |
| 252 | 5-bromo-6-fluoro-4-(8-fluoro-4-(((1S,2S)-2-fluorocyclopropyl)(methyl)amino)-2-(((2R,7aS)-2- |
| | methoxytetrahydro-1H-pyrrolizin-7a(5H)-yl)methoxy)pyrido[4,3-d]pyrimidin-7-yl)naphthalen-2-ol; |
| 253 | 5-ethyl-4-(8-fluoro-4-(((1S,2S)-2-fluorocyclopropyl)(methyl-d3)amino)-2-(((2R,7aS)-2-methoxytetrahydro- |
| | 1H-pyrrolizin-7a(5H)-yl)methoxy)pyrido[4,3-d]pyrimidin-7-yl)naphthalen-2-ol; |
| 254 | 5-ethyl-4-(4-(ethyl((1S,2S)-2-fluorocyclopropyl)amino)-8-fluoro-2-(((2R,7aS)-2-methoxytetrahydro- |
| | 1H-pyrrolizin-7a(5H)-yl)methoxy)pyrido[4,3-d]pyrimidin-7-yl)naphthalen-2-ol; |
| 255 | 5-ethyl-4-(8-fluoro-4-(((1S,2S)-2-fluorocyclopropyl)(methyl)amino)-2-(((2R,7aS)-2-methoxytetrahydro- |
| | 1H-pyrrolizin-7a(5H)-yl)methoxy)pyrido[4,3-d]pyrimidin-7-yl)naphthalen-2-ol; |
| 256 | 5,6-difluoro-4-(8-fluoro-4-(((1S,2S)-2-fluorocyclopropyl)(methyl-d3)amino)-2-(((2R,7aS)-2- |
| | methoxytetrahydro-1H-pyrrolizin-7a(5H)-yl)methoxy)pyrido[4,3-d]pyrimidin-7-yl)naphthalen-2-ol; |
| 257 | 4-(4-(ethyl((1S,2S)-2-fluorocyclopropyl)amino)-8-fluoro-2-(((2R,7aS)-2-methoxytetrahydro-1H- |
| | pyrrolizin-7a(5H)-yl)methoxy)pyrido[4,3-d]pyrimidin-7-yl)-5,6-difluoronaphthalen-2-ol; |
| 258 | 5,6-difluoro-4-(8-fluoro-4-(((1S,2S)-2-fluorocyclopropyl)(methyl)amino)-2-(((2R,7aS)-2- |
| | methoxytetrahydro-1H-pyrrolizin-7a(5H)-yl)methoxy)pyrido[4,3-d]pyrimidin-7-yl)naphthalen-2-ol; |
| 259 | 5-chloro-6-fluoro-4-(8-fluoro-4-(((1S,2S)-2-fluorocyclopropyl)(methyl-d3)amino)-2-(((2R,7aS)-2- |
| | methoxytetrahydro-1H-pyrrolizin-7a(5H)-yl)methoxy)pyrido[4,3-d]pyrimidin-7-yl)naphthalen-2-ol; |
| 260 | 5-chloro-4-(4-(ethyl((1S,2S)-2-fluorocyclopropyl)amino)-8-fluoro-2-(((2R,7aS)-2-methoxytetrahydro- |
| | 1H-pyrrolizin-7a(5H)-yl)methoxy)pyrido[4,3-d]pyrimidin-7-yl)-6-fluoronaphthalen-2-ol; |
| 261 | 5-chloro-6-fluoro-4-(8-fluoro-4-(((1S,2S)-2-fluorocyclopropyl)(methyl)amino)-2-(((2R,7aS)-2- |
| | methoxytetrahydro-1H-pyrrolizin-7a(5H)-yl)methoxy)pyrido[4,3-d]pyrimidin-7-yl)naphthalen-2-ol; |
| 262 | 5-ethyl-6-fluoro-4-(8-fluoro-4-(((1S,2S)-2-fluorocyclopropyl)(methyl-d3)amino)-2-(((2R,7aS)-2- |
| | methoxytetrahydro-1H-pyrrolizin-7a(5H)-yl)methoxy)pyrido[4,3-d]pyrimidin-7-yl)naphthalen-2-ol; |
| 263 | 5-ethyl-4-(4-(ethyl((1S,2S)-2-fluorocyclopropyl)amino)-8-fluoro-2-(((2R,7aS)-2-methoxytetrahydro- |
| | 1H-pyrrolizin-7a(5H)-yl)methoxy)pyrido[4,3-d]pyrimidin-7-yl)-6-fluoronaphthalen-2-ol; |
| 264 | 5-ethyl-6-fluoro-4-(8-fluoro-4-(((1S,2S)-2-fluorocyclopropyl)(methyl)amino)-2-(((2R,7aS)-2- |
| | methoxytetrahydro-1H-pyrrolizin-7a(5H)-yl)methoxy)pyrido[4,3-d]pyrimidin-7-yl)naphthalen-2-ol; |
| 265 | 5-ethynyl-4-(8-fluoro-4-(((1S,2S)-2-fluorocyclopropyl)(methyl-d3)amino)-2-(((2R,7aS)-2- |
| | methoxytetrahydro-1H-pyrrolizin-7a(5H)-yl)methoxy)pyrido[4,3-d]pyrimidin-7-yl)naphthalen-2-ol; |
| 266 | 4-(4-(ethyl((1S,2S)-2-fluorocyclopropyl)amino)-8-fluoro-2-(((2R,7aS)-2-methoxytetrahydro-1H- |
| | pyrrolizin-7a(5H)-yl)methoxy)pyrido[4,3-d]pyrimidin-7-yl)-5-ethynylnaphthalen-2-ol; |
| 267 | 5-ethynyl-4-(8-fluoro-4-(((1S,2S)-2-fluorocyclopropyl)(methyl)amino)-2-(((2R,7aS)-2- |
| | methoxytetrahydro-1H-pyrrolizin-7a(5H)-yl)methoxy)pyrido[4,3-d]pyrimidin-7-yl)naphthalen-2-ol; |
| 268 | 5-ethynyl-6-fluoro-4-(8-fluoro-4-(((1S,2S)-2-fluorocyclopropyl)(methyl-d3)amino)-2-(((2R,7aS)-2- |
| | fluorotetrahydro-1H-pyrrolizin-7a(5H)-yl-2-d)methoxy-d2)pyrido[4,3-d]pyrimidin-7-yl)naphthalen-2-ol; |
| 269 | 4-(4-(ethyl((1S,2S)-2-fluorocyclopropyl)amino)-8-fluoro-2-(((2R,7aS)-2-fluorotetrahydro-1H-pyrrolizin- |
| | 7a(5H)-yl-2-d)methoxy-d2)pyrido[4,3-d]pyrimidin-7-yl)-5-ethynyl-6-fluoronaphthalen-2-ol; |
| 270 | 5-ethynyl-6-fluoro-4-(8-fluoro-4-(((1S,2S)-2-fluorocyclopropyl)(methyl)amino)-2-(((2R,7aS)-2- |
| | fluorotetrahydro-1H-pyrrolizin-7a(5H)-yl-2-d)methoxy-d2)pyrido[4,3-d]pyrimidin-7-yl)naphthalen-2-ol; |
| 271 | 4-(8-fluoro-4-(((1S,2S)-2-fluorocyclopropyl)(methyl-d3)amino)-2-(((2R,7aS)-2-fluorotetrahydro-1H- |
| | pyrrolizin-7a(5H)-yl-2-d)methoxy-d2)pyrido[4,3-d]pyrimidin-7-yl)-5-(methylthio)naphthalen-2-ol; |
| 272 | 4-(4-(ethyl((1S,2S)-2-fluorocyclopropyl)amino)-8-fluoro-2-(((2R,7aS)-2-fluorotetrahydro-1H-pyrrolizin- |
| | 7a(5H)-yl-2-d)methoxy-d2)pyrido[4,3-d]pyrimidin-7-yl)-5-(methylthio)naphthalen-2-ol; |
| 273 | 4-(8-fluoro-4-(((1S,2S)-2-fluorocyclopropyl)(methyl)amino)-2-(((2R,7aS)-2-fluorotetrahydro-1H-pyrrolizin- |
| | 7a(5H)-yl-2-d)methoxy-d2)pyrido[4,3-d]pyrimidin-7-yl)-5-(methylthio)naphthalen-2-ol; |
| 274 | 5-bromo-6-fluoro-4-(8-fluoro-4-(((1S,2S)-2-fluorocyclopropyl)(methyl-d3)amino)-2-(((2R,7aS)-2- |
| | fluorotetrahydro-1H-pyrrolizin-7a(5H)-yl-2-d)methoxy-d2)pyrido[4,3-d]pyrimidin-7-yl)naphthalen-2-ol; |
| 275 | 5-bromo-4-(4-(ethyl((1S,2S)-2-fluorocyclopropyl)amino)-8-fluoro-2-(((2R,7aS)-2-fluorotetrahydro-1H- |
| | pyrrolizin-7a(5H)-yl-2-d)methoxy-d2)pyrido[4,3-d]pyrimidin-7-yl)-6-fluoronaphthalen-2-ol; |
| 276 | 5-bromo-6-fluoro-4-(8-fluoro-4-(((1S,2S)-2-fluorocyclopropyl)(methyl)amino)-2-(((2R,7aS)-2- |
| | fluorotetrahydro-1H-pyrrolizin-7a(5H)-yl-2-d)methoxy-d2)pyrido[4,3-d]pyrimidin-7-yl)naphthalen-2-ol; |
| 277 | 5-ethyl-4-(8-fluoro-4-(((1S,2S)-2-fluorocyclopropyl)(methyl-d3)amino)-2-(((2R,7aS)-2-fluorotetrahydro- |
| | 1H-pyrrolizin-7a(5H)-yl-2-d)methoxy-d2)pyrido[4,3-d]pyrimidin-7-yl)naphthalen-2-ol; |
| 278 | 5-ethyl-4-(4-(ethyl((1S,2S)-2-fluorocyclopropyl)amino)-8-fluoro-2-(((2R,7aS)-2-fluorotetrahydro- |
| | 1H-pyrrolizin-7a(5H)-yl-2-d)methoxy-d2)pyrido[4,3-d]pyrimidin-7-yl)naphthalen-2-ol; |
| 279 | 5-ethyl-4-(8-fluoro-4-(((1S,2S)-2-fluorocyclopropyl)(methyl)amino)-2-(((2R,7aS)-2-fluorotetrahydro- |
| | 1H-pyrrolizin-7a(5H)-yl-2-d)methoxy-d2)pyrido[4,3-d]pyrimidin-7-yl)naphthalen-2-ol; |
| 280 | 5,6-difluoro-4-(8-fluoro-4-(((1S,2S)-2-fluorocyclopropyl)(methyl-d3)amino)-2-(((2R,7aS)-2- |
| | fluorotetrahydro-1H-pyrrolizin-7a(5H)-yl-2-d)methoxy-d2)pyrido[4,3-d]pyrimidin-7-yl)naphthalen-2-ol; |
| 281 | 4-(4-(ethyl((1S,2S)-2-fluorocyclopropyl)amino)-8-fluoro-2-(((2R,7aS)-2-fluorotetrahydro-1H-pyrrolizin- |
| | 7a(5H)-yl-2-d)methoxy-d2)pyrido[4,3-d]pyrimidin-7-yl)-5,6-difluoronaphthalen-2-ol; |
| 282 | 5,6-difluoro-4-(8-fluoro-4-(((1S,2S)-2-fluorocyclopropyl)(methyl)amino)-2-(((2R,7aS)-2-fluorotetrahydro- |
| | 1H-pyrrolizin-7a(5H)-yl-2-d)methoxy-d2)pyrido[4,3-d]pyrimidin-7-yl)naphthalen-2-ol; |
| 283 | 5-chloro-6-fluoro-4-(8-fluoro-4-(((1S,2S)-2-fluorocyclopropyl)(methyl-d3)amino)-2-(((2R,7aS)-2- |
| | fluorotetrahydro-1H-pyrrolizin-7a(5H)-yl-2-d)methoxy-d2)pyrido[4,3-d]pyrimidin-7-yl)naphthalen-2-ol; |
| 284 | 5-chloro-4-(4-(ethyl((1S,2S)-2-fluorocyclopropyl)amino)-8-fluoro-2-(((2R,7aS)-2-fluorotetrahydro-1 |
| | H-pyrrolizin-7a(5H)-yl-2-d)methoxy-d2)pyrido[4,3-d]pyrimidin-7-yl)-6-fluoronaphthalen-2-ol; |
| 285 | 5-chloro-6-fluoro-4-(8-fluoro-4-(((1S,2S)-2-fluorocyclopropyl)(methyl)amino)-2-(((2R,7aS)-2- |
| | fluorotetrahydro-1H-pyrrolizin-7a(5H)-yl-2-d)methoxy-d2)pyrido[4,3-d]pyrimidin-7-yl)naphthalen-2-ol; |
| 286 | 5-ethyl-6-fluoro-4-(8-fluoro-4-(((1S,2S)-2-fluorocyclopropyl)(methyl-d3)amino)-2-(((2R,7aS)-2- |
| | fluorotetrahydro-1H-pyrrolizin-7a(5H)-yl-2-d)methoxy-d2)pyrido[4,3-d]pyrimidin-7-yl)naphthalen-2-ol; |
| 287 | 5-ethyl-4-(4-(ethyl((1S,2S)-2-fluorocyclopropyl)amino)-8-fluoro-2-(((2R,7aS)-2-fluorotetrahydro- |
| | 1H-pyrrolizin-7a(5H)-yl-2-d)methoxy-d2)pyrido[4,3-d]pyrimidin-7-yl)-6-fluoronaphthalen-2-ol; |
| 288 | 5-ethyl-6-fluoro-4-(8-fluoro-4-(((1S,2S)-2-fluorocyclopropyl)(methyl)amino)-2-(((2R,7aS)-2- |
| | fluorotetrahydro-1H-pyrrolizin-7a(5H)-yl-2-d)methoxy-d2)pyrido[4,3-d]pyrimidin-7-yl)naphthalen-2-ol; |
| 289 | 5-ethynyl-4-(8-fluoro-4-(((1S,2S)-2-fluorocyclopropyl)(methyl-d3)amino)-2-(((2R,7aS)-2-fluorotetrahydro- |
| | 1H-pyrrolizin-7a(5H)-yl-2-d)methoxy-d2)pyrido[4,3-d]pyrimidin-7-yl)naphthalen-2-ol; |
| 290 | 4-(4-(ethyl((1S,2S)-2-fluorocyclopropyl)amino)-8-fluoro-2-(((2R,7aS)-2-fluorotetrahydro-1H-pyrrolizin- |
| | 7a(5H)-yl-2-d)methoxy-d2)pyrido[4,3-d]pyrimidin-7-yl)-5-ethynylnaphthalen-2-ol; |
| 291 | 5-ethynyl-4-(8-fluoro-4-(((1S,2S)-2-fluorocyclopropyl)(methyl)amino)-2-(((2R,7aS)-2-fluorotetrahydro- |
| | 1H-pyrrolizin-7a(5H)-yl-2-d)methoxy-d2)pyrido[4,3-d]pyrimidin-7-yl)naphthalen-2-ol; |
| 292 | 4-(4-(ethyl((1S,2S)-2-fluorocyclopropyl)amino)-8-fluoro-2-(((2R,7aS)-2-fluorotetrahydro-1H-pyrrolizin- |
| | 7a(5H)-yl-2,5,5-d3)methoxy-d2)pyrido[4,3-d]pyrimidin-7-yl)-5-ethynyl-6-fluoronaphthalen-2-ol; |
| 293 | 4-(8-fluoro-4-(((1S,2S)-2-fluorocyclopropyl)(methyl-d3)amino)-2-(((2R,7aS)-2-fluorotetrahydro-1H- |
| | pyrrolizin-7a(5H)-yl-2,5,5-d3)methoxy-d2)pyrido[4,3-d]pyrimidin-7-yl)-5-(methylthio)naphthalen-2-ol; |
| 294 | 4-(4-(ethyl((1S,2S)-2-fluorocyclopropyl)amino)-8-fluoro-2-(((2R,7aS)-2-fluorotetrahydro-1H-pyrrolizin- |
| | 7a(5H)-yl-2,5,5-d3)methoxy-d2)pyrido[4,3-d]pyrimidin-7-yl)-5-(methylthio)naphthalen-2-ol; |
| 295 | 4-(8-fluoro-4-(((1S,2S)-2-fluorocyclopropyl)(methyl)amino)-2-(((2R,7aS)-2-fluorotetrahydro-1H- |
| | pyrrolizin-7a(5H)-yl-2,5,5-d3)methoxy-d2)pyrido[4,3-d]pyrimidin-7-yl)-5-(methylthio)naphthalen-2-ol; |
| 296 | 5-bromo-6-fluoro-4-(8-fluoro-4-(((1S,2S)-2-fluorocyclopropyl)(methyl-d3)amino)-2-(((2R,7aS)-2- |
| | fluorotetrahydro-1H-pyrrolizin-7a(5H)-yl-2,5,5-d3)methoxy-d2)pyrido[4,3-d]pyrimidin-7-yl)naphthalen-2-ol; |
| 297 | 5-bromo-4-(4-(ethyl((1S,2S)-2-fluorocyclopropyl)amino)-8-fluoro-2-(((2R,7aS)-2-fluorotetrahydro-1H- |
| | pyrrolizin-7a(5H)-yl-2,5,5-d3)methoxy-d2)pyrido[4,3-d]pyrimidin-7-yl)-6-fluoronaphthalen-2-ol; |
| 298 | 5-bromo-6-fluoro-4-(8-fluoro-4-(((1S,2S)-2-fluorocyclopropyl)(methyl)amino)-2-(((2R,7aS)-2- |
| | fluorotetrahydro-1H-pyrrolizin-7a(5H)-yl-2,5,5-d3)methoxy-d2)pyrido[4,3-d]pyrimidin-7-yl)naphthalen-2-ol; |
| 299 | 5-ethyl-4-(8-fluoro-4-(((1S,2S)-2-fluorocyclopropyl)(methyl-d3)amino)-2-(((2R,7aS)-2-fluorotetrahydro- |
| | 1H-pyrrolizin-7a(5H)-yl-2,5,5-d3)methoxy-d2)pyrido[4,3-d]pyrimidin-7-yl)naphthalen-2-ol; |
| 300 | 5-ethyl-4-(4-(ethyl((1S,2S)-2-fluorocyclopropyl)amino)-8-fluoro-2-(((2R,7aS)-2-fluorotetrahydro- |
| | 1H-pyrrolizin-7a(5H)-yl-2,5,5-d3)methoxy-d2)pyrido[4,3-d]pyrimidin-7-yl)naphthalen-2-ol; |
| 301 | 5-ethyl-4-(8-fluoro-4-(((1S,2S)-2-fluorocyclopropyl)(methyl)amino)-2-(((2R,7aS)-2-fluorotetrahydro- |
| | 1H-pyrrolizin-7a(5H)-yl-2,5,5-d3)methoxy-d2)pyrido[4,3-d]pyrimidin-7-yl)naphthalen-2-ol; |
| 302 | 5,6-difluoro-4-(8-fluoro-4-(((1S,2S)-2-fluorocyclopropyl)(methyl-d3)amino)-2-(((2R,7aS)-2-fluorotetrahydro- |
| | 1H-pyrrolizin-7a(5H)-yl-2,5,5-d3)methoxy-d2)pyrido[4,3-d]pyrimidin-7-yl)naphthalen-2-ol; |
| 303 | 4-(4-(ethyl((1S,2S)-2-fluorocyclopropyl)amino)-8-fluoro-2-(((2R,7aS)-2-fluorotetrahydro-1H-pyrrolizin- |
| | 7a(5H)-yl-2,5,5-d3)methoxy-d2)pyrido[4,3-d]pyrimidin-7-yl)-5,6-difluoronaphthalen-2-ol; |
| 304 | 5,6-difluoro-4-(8-fluoro-4-(((1S,2S)-2-fluorocyclopropyl)(methyl)amino)-2-(((2R,7aS)-2-fluorotetrahydro- |
| | 1H-pyrrolizin-7a(5H)-yl-2,5,5-d3)methoxy-d2)pyrido[4,3-d]pyrimidin-7-yl)naphthalen-2-ol; |
| 305 | 5-chloro-6-fluoro-4-(8-fluoro-4-(((1S,2S)-2-fluorocyclopropyl)(methyl-d3)amino)-2-(((2R,7aS)-2- |
| | fluorotetrahydro-1H-pyrrolizin-7a(5H)-yl-2,5,5-d3)methoxy-d2)pyrido[4,3-d]pyrimidin-7-yl)naphthalen-2-ol; |
| 306 | 5-chloro-4-(4-(ethyl((1S,2S)-2-fluorocyclopropyl)amino)-8-fluoro-2-(((2R,7aS)-2-fluorotetrahydro- |
| | 1H-pyrrolizin-7a(5H)-yl-2,5,5-d3)methoxy-d2)pyrido[4,3-d]pyrimidin-7-yl)-6-fluoronaphthalen-2-ol; |
| 307 | 5-chloro-6-fluoro-4-(8-fluoro-4-(((1S,2S)-2-fluorocyclopropyl)(methyl)amino)-2-(((2R,7aS)-2-fluorotetrahydro- |
| | 1H-pyrrolizin-7a(5H)-yl-2,5,5-d3)methoxy-d2)pyrido[4,3-d]pyrimidin-7-yl)naphthalen-2-ol; |
| 308 | 5-ethyl-6-fluoro-4-(8-fluoro-4-(((1S,2S)-2-fluorocyclopropyl)(methyl-d3)amino)-2-(((2R,7aS)-2- |
| | fluorotetrahydro-1H-pyrrolizin-7a(5H)-yl-2,5,5-d3)methoxy-d2)pyrido[4,3-d]pyrimidin-7-yl)naphthalen-2-ol; |
| 309 | 5-ethyl-4-(4-(ethyl((1S,2S)-2-fluorocyclopropyl)amino)-8-fluoro-2-(((2R,7aS)-2-fluorotetrahydro- |
| | 1H-pyrrolizin-7a(5H)-yl-2,5,5-d3)methoxy-d2)pyrido[4,3-d]pyrimidin-7-yl)-6-fluoronaphthalen-2-ol; |
| 310 | 5-ethyl-6-fluoro-4-(8-fluoro-4-(((1S,2S)-2-fluorocyclopropyl)(methyl)amino)-2-(((2R,7aS)-2-fluorotetrahydro- |
| | 1H-pyrrolizin-7a(5H)-yl-2,5,5-d3)methoxy-d2)pyrido[4,3-d]pyrimidin-7-yl)naphthalen-2-ol; |
| 311 | 5-ethynyl-4-(8-fluoro-4-(((1S,2S)-2-fluorocyclopropyl)(methyl-d3)amino)-2-(((2R,7aS)-2-fluorotetrahydro- |
| | 1H-pyrrolizin-7a(5H)-yl-2,5,5-d3)methoxy-d2)pyrido[4,3-d]pyrimidin-7-yl)naphthalen-2-ol; |
| 312 | 4-(4-(ethyl((1S,2S)-2-fluorocyclopropyl)amino)-8-fluoro-2-(((2R,7aS)-2-fluorotetrahydro-1H-pyrrolizin- |
| | 7a(5H)-yl-2,5,5-d3)methoxy-d2)pyrido[4,3-d]pyrimidin-7-yl)-5-ethynylnaphthalen-2-ol; |
| 313 | 5-ethynyl-4-(8-fluoro-4-(((1S,2S)-2-fluorocyclopropyl)(methyl)amino)-2-(((2R,7aS)-2-fluorotetrahydro- |
| | 1H-pyrrolizin-7a(5H)-yl-2,5,5-d3)methoxy-d2)pyrido[4,3-d]pyrimidin-7-yl)naphthalen-2-ol; |
| 314 | 5-ethynyl-6-fluoro-4-(8-fluoro-2-(((2R,7aS)-2-fluoro-2-methyltetrahydro-1H-pyrrolizin-7a(5H)-yl)methoxy)- |
| | 4-(((1S,2S)-2-fluorocyclopropyl)(methyl-d3)amino)pyrido[4,3-d]pyrimidin-7-yl)naphthalen-2-ol; |
| 315 | 4-(4-(ethyl((1S,2S)-2-fluorocyclopropyl)amino)-8-fluoro-2-(((2R,7aS)-2-fluoro-2-methyltetrahydro- |
| | 1H-pyrrolizin-7a(5H)-yl)methoxy)pyrido[4,3-d]pyrimidin-7-yl)-5-ethynyl-6-fluoronaphthalen-2-ol; |
| 316 | 4-(8-fluoro-2-(((2R,7aS)-2-fluoro-2-methyltetrahydro-1H-pyrrolizin-7a(5H)-yl)methoxy)-4-(((1S,2S)- |
| | 2-fluorocyclopropyl)(methyl-d3)amino)pyrido[4,3-d]pyrimidin-7-yl)-5-(methylthio)naphthalen-2-ol; |
| 317 | 4-(4-(ethyl((1S,2S)-2-fluorocyclopropyl)amino)-8-fluoro-2-(((2R,7aS)-2-fluoro-2-methyltetrahydro- |
| | 1H-pyrrolizin-7a(5H)-yl)methoxy)pyrido[4,3-d]pyrimidin-7-yl)-5-(methylthio)naphthalen-2-ol; |
| 318 | 4-(8-fluoro-2-(((2R,7aS)-2-fluoro-2-methyltetrahydro-1H-pyrrolizin-7a(5H)-yl)methoxy)-4-(((1S,2S)- |
| | 2-fluorocyclopropyl)(methyl)amino)pyrido[4,3-d]pyrimidin-7-yl)-5-(methylthio)naphthalen-2-ol; |
| 319 | 5-bromo-6-fluoro-4-(8-fluoro-2-(((2R,7aS)-2-fluoro-2-methyltetrahydro-1H-pyrrolizin-7a(5H)-yl)methoxy)- |
| | 4-(((1S,2S)-2-fluorocyclopropyl)(methyl-d3)amino)pyrido[4,3-d]pyrimidin-7-yl)naphthalen-2-ol; |
| 320 | 5-bromo-4-(4-(ethyl((1S,2S)-2-fluorocyclopropyl)amino)-8-fluoro-2-(((2R,7aS)-2-fluoro-2-methyltetrahydro- |
| | 1H-pyrrolizin-7a(5H)-yl)methoxy)pyrido[4,3-d]pyrimidin-7-yl)-6-fluoronaphthalen-2-ol; |
| 321 | 5-bromo-6-fluoro-4-(8-fluoro-2-(((2R,7aS)-2-fluoro-2-methyltetrahydro-1H-pyrrolizin-7a(5H)-yl)methoxy)- |
| | 4-(((1S,2S)-2-fluorocyclopropyl)(methyl)amino)pyrido[4,3-d]pyrimidin-7-yl)naphthalen-2-ol; |
| 322 | 5-ethyl-4-(8-fluoro-2-(((2R,7aS)-2-fluoro-2-methyltetrahydro-1H-pyrrolizin-7a(5H)-yl)methoxy)-4-(((1S,2S)- |
| | 2-fluorocyclopropyl)(methyl-d3)amino)pyrido[4,3-d]pyrimidin-7-yl)naphthalen-2-ol; |
| 323 | 5-ethyl-4-(4-(ethyl((1S,2S)-2-fluorocyclopropyl)amino)-8-fluoro-2-(((2R,7aS)-2-fluoro-2-methyltetrahydro- |
| | 1H-pyrrolizin-7a(5H)-yl)methoxy)pyrido[4,3-d]pyrimidin-7-yl)naphthalen-2-ol; |
| 324 | 5-ethyl-4-(8-fluoro-2-(((2R,7aS)-2-fluoro-2-methyltetrahydro-1H-pyrrolizin-7a(5H)-yl)methoxy)-4-(((1S,2S)- |
| | 2-fluorocyclopropyl)(methyl)amino)pyrido[4,3-d]pyrimidin-7-yl)naphthalen-2-ol; |
| 325 | 5,6-difluoro-4-(8-fluoro-2-(((2R,7aS)-2-fluoro-2-methyltetrahydro-1H-pyrrolizin-7a(5H)-yl)methoxy)-4- |
| | (((1S,2S)-2-fluorocyclopropyl)(methyl-d3)amino)pyrido[4,3-d]pyrimidin-7-yl)naphthalen-2-ol; |
| 326 | 4-(4-(ethyl((1S,2S)-2-fluorocyclopropyl)amino)-8-fluoro-2-(((2R,7aS)-2-fluoro-2-methyltetrahydro-1H- |
| | pyrrolizin-7a(5H)-yl)methoxy)pyrido[4,3-d]pyrimidin-7-yl)-5,6-difluoronaphthalen-2-ol; |
| 327 | 5,6-difluoro-4-(8-fluoro-2-(((2R,7aS)-2-fluoro-2-methyltetrahydro-1H-pyrrolizin-7a(5H)-yl)methoxy)-4- |
| | (((1S,2S)-2-fluorocyclopropyl)(methyl)amino)pyrido[4,3-d]pyrimidin-7-yl)naphthalen-2-ol; |
| 328 | 5-chloro-6-fluoro-4-(8-fluoro-2-(((2R,7aS)-2-fluoro-2-methyltetrahydro-1H-pyrrolizin-7a(5H)-yl)methoxy)- |
| | 4-(((1S,2S)-2-fluorocyclopropyl)(methyl-d3)amino)pyrido[4,3-d]pyrimidin-7-yl)naphthalen-2-ol; |
| 329 | 5-chloro-4-(4-(ethyl((1S,2S)-2-fluorocyclopropyl)amino)-8-fluoro-2-(((2R,7aS)-2-fluoro-2-methyltetrahydro- |
| | 1H-pyrrolizin-7a(5H)-yl)methoxy)pyrido[4,3-d]pyrimidin-7-yl)-6-fluoronaphthalen-2-ol; |
| 330 | 5-chloro-6-fluoro-4-(8-fluoro-2-(((2R,7aS)-2-fluoro-2-methyltetrahydro-1H-pyrrolizin-7a(5H)-yl)methoxy)- |
| | 4-(((1S,2S)-2-fluorocyclopropyl)(methyl)amino)pyrido[4,3-d]pyrimidin-7-yl)naphthalen-2-ol; |
| 331 | 5-ethyl-6-fluoro-4-(8-fluoro-2-(((2R,7aS)-2-fluoro-2-methyltetrahydro-1H-pyrrolizin-7a(5H)-yl)methoxy)- |
| | 4-(((1S,2S)-2-fluorocyclopropyl)(methyl-d3)amino)pyrido[4,3-d]pyrimidin-7-yl)naphthalen-2-ol; |
| 332 | 5-ethyl-4-(4-(ethyl((1S,2S)-2-fluorocyclopropyl)amino)-8-fluoro-2-(((2R,7aS)-2-fluoro-2-methyltetrahydro- |
| | 1H-pyrrolizin-7a(5H)-yl)methoxy)pyrido[4,3-d]pyrimidin-7-yl)-6-fluoronaphthalen-2-ol; |
| 333 | 5-ethyl-6-fluoro-4-(8-fluoro-2-(((2R,7aS)-2-fluoro-2-methyltetrahydro-1H-pyrrolizin-7a(5H)-yl)methoxy)- |
| | 4-(((1S,2S)-2-fluorocyclopropyl)(methyl)amino)pyrido[4,3-d]pyrimidin-7-yl)naphthalen-2-ol; |
| 334 | 5-ethynyl-4-(8-fluoro-2-(((2R,7aS)-2-fluoro-2-methyltetrahydro-1H-pyrrolizin-7a(5H)-yl)methoxy)-4- |
| | (((1S,2S)-2-fluorocyclopropyl)(methyl-d3)amino)pyrido[4,3-d]pyrimidin-7-yl)naphthalen-2-ol; |
| 335 | 4-(4-(ethyl((1S,2S)-2-fluorocyclopropyl)amino)-8-fluoro-2-(((2R,7aS)-2-fluoro-2-methyltetrahydro- |
| | 1H-pyrrolizin-7a(5H)-yl)methoxy)pyrido[4,3-d]pyrimidin-7-yl)-5-ethynylnaphthalen-2-ol; |
| 336 | 5-ethynyl-4-(8-fluoro-2-(((2R,7aS)-2-fluoro-2-methyltetrahydro-1H-pyrrolizin-7a(5H)-yl)methoxy)- |
| | 4-(((1S,2S)-2-fluorocyclopropyl)(methyl)amino)pyrido[4,3-d]pyrimidin-7-yl)naphthalen-2-ol; |
| 337 | 4-(4-(ethyl((1S,2S)-2-fluorocyclopropyl)amino)-8-fluoro-2-(((2R,7aS)-2-fluorotetrahydro-1H-pyrrolizin- |
| | 7a(5H)-yl-5,5-d2)methoxy-d2)pyrido[4,3-d]pyrimidin-7-yl)-5-ethynyl-6-fluoronaphthalen-2-ol; |
| 338 | 4-(8-fluoro-4-(((1S,2S)-2-fluorocyclopropyl)(methyl-d3)amino)-2-(((2R,7aS)-2-fluorotetrahydro-1H- |
| | pyrrolizin-7a(5H)-yl-5,5-d2)methoxy-d2)pyrido[4,3-d]pyrimidin-7-yl)-5-(methylthio)naphthalen-2-ol; |
| 339 | 4-(4-(ethyl((1S,2S)-2-fluorocyclopropyl)amino)-8-fluoro-2-(((2R,7aS)-2-fluorotetrahydro-1H-pyrrolizin- |
| | 7a(5H)-yl-5,5-d2)methoxy-d2)pyrido[4,3-d]pyrimidin-7-yl)-5-(methylthio)naphthalen-2-ol; |
| 340 | 4-(8-fluoro-4-(((1S,2S)-2-fluorocyclopropyl)(methyl)amino)-2-(((2R,7aS)-2-fluorotetrahydro-1H- |
| | pyrrolizin-7a(5H)-yl-5,5-d2)methoxy-d2)pyrido[4,3-d]pyrimidin-7-yl)-5-(methylthio)naphthalen-2-ol; |
| 341 | 5-bromo-6-fluoro-4-(8-fluoro-4-(((1S,2S)-2-fluorocyclopropyl)(methyl-d3)amino)-2-(((2R,7aS)-2- |
| | fluorotetrahydro-1H-pyrrolizin-7a(5H)-yl-5,5-d2)methoxy-d2)pyrido[4,3-d]pyrimidin-7-yl)naphthalen-2-ol; |
| 342 | 5-bromo-4-(4-(ethyl((1S,2S)-2-fluorocyclopropyl)amino)-8-fluoro-2-(((2R,7aS)-2-fluorotetrahydro- |
| | 1H-pyrrolizin-7a(5H)-yl-5,5-d2)methoxy-d2)pyrido[4,3-d]pyrimidin-7-yl)-6-fluoronaphthalen-2-ol; |
| 343 | 5-bromo-6-fluoro-4-(8-fluoro-4-(((1S,2S)-2-fluorocyclopropyl)(methyl)amino)-2-(((2R,7aS)-2- |
| | fluorotetrahydro-1H-pyrrolizin-7a(5H)-yl-5,5-d2)methoxy-d2)pyrido[4,3-d]pyrimidin-7-yl)naphthalen-2-ol; |
| 344 | 5-ethyl-4-(8-fluoro-4-(((1S,2S)-2-fluorocyclopropyl)(methyl-d3)amino)-2-(((2R,7aS)-2-fluorotetrahydro- |
| | 1H-pyrrolizin-7a(5H)-yl-5,5-d2)methoxy-d2)pyrido[4,3-d]pyrimidin-7-yl)naphthalen-2-ol; |
| 345 | 5-ethyl-4-(4-(ethyl((1S,2S)-2-fluorocyclopropyl)amino)-8-fluoro-2-(((2R,7aS)-2-fluorotetrahydro-1H- |
| | pyrrolizin-7a(5H)-yl-5,5-d2)methoxy-d2)pyrido[4,3-d]pyrimidin-7-yl)naphthalen-2-ol; |
| 346 | 5-ethyl-4-(8-fluoro-4-(((1S,2S)-2-fluorocyclopropyl)(methyl)amino)-2-(((2R,7aS)-2-fluorotetrahydro-1H- |
| | pyrrolizin-7a(5H)-yl-5,5-d2)methoxy-d2)pyrido[4,3-d]pyrimidin-7-yl)naphthalen-2-ol; |
| 347 | 5,6-difluoro-4-(8-fluoro-4-(((1S,2S)-2-fluorocyclopropyl)(methyl-d3)amino)-2-(((2R,7aS)-2-fluorotetrahydro- |
| | 1H-pyrrolizin-7a(5H)-yl-5,5-d2)methoxy-d2)pyrido[4,3-d]pyrimidin-7-yl)naphthalen-2-ol; |
| 348 | 4-(4-(ethyl((1S,2S)-2-fluorocyclopropyl)amino)-8-fluoro-2-(((2R,7aS)-2-fluorotetrahydro-1H-pyrrolizin- |
| | 7a(5H)-yl-5,5-d2)methoxy-d2)pyrido[4,3-d]pyrimidin-7-yl)-5,6-difluoronaphthalen-2-ol; |
| 349 | 5,6-difluoro-4-(8-fluoro-4-(((1S,2S)-2-fluorocyclopropyl)(methyl)amino)-2-(((2R,7aS)-2-fluorotetrahydro- |
| | 1H-pyrrolizin-7a(5H)-yl-5,5-d2)methoxy-d2)pyrido[4,3-d]pyrimidin-7-yl)naphthalen-2-ol; |
| 350 | 5-chloro-6-fluoro-4-(8-fluoro-4-(((1S,2S)-2-fluorocyclopropyl)(methyl-d3)amino)-2-(((2R,7aS)-2- |
| | fluorotetrahydro-1H-pyrrolizin-7a(5H)-yl-5,5-d2)methoxy-d2)pyrido[4,3-d]pyrimidin-7-yl)naphthalen-2-ol; |
| 351 | 5-chloro-4-(4-(ethyl((1S,2S)-2-fluorocyclopropyl)amino)-8-fluoro-2-(((2R,7aS)-2-fluorotetrahydro- |
| | 1H-pyrrolizin-7a(5H)-yl-5,5-d2)methoxy-d2)pyrido[4,3-d]pyrimidin-7-yl)-6-fluoronaphthalen-2-ol; |
| 352 | 5-chloro-6-fluoro-4-(8-fluoro-4-(((1S,2S)-2-fluorocyclopropyl)(methyl)amino)-2-(((2R,7aS)-2- |
| | fluorotetrahydro-1H-pyrrolizin-7a(5H)-yl-5,5-d2)methoxy-d2)pyrido[4,3-d]pyrimidin-7-yl)naphthalen-2-ol; |
| 353 | 5-ethyl-6-fluoro-4-(8-fluoro-4-(((1S,2S)-2-fluorocyclopropyl)(methyl-d3)amino)-2-(((2R,7aS)-2- |
| | fluorotetrahydro-1H-pyrrolizin-7a(5H)-yl-5,5-d2)methoxy-d2)pyrido[4,3-d]pyrimidin-7-yl)naphthalen-2-ol; |
| 354 | 5-ethyl-4-(4-(ethyl((1S,2S)-2-fluorocyclopropyl)amino)-8-fluoro-2-(((2R,7aS)-2-fluorotetrahydro- |
| | 1H-pyrrolizin-7a(5H)-yl-5,5-d2)methoxy-d2)pyrido[4,3-d]pyrimidin-7-yl)-6-fluoronaphthalen-2-ol; |
| 355 | 5-ethyl-6-fluoro-4-(8-fluoro-4-(((1S,2S)-2-fluorocyclopropyl)(methyl)amino)-2-(((2R,7aS)-2- |
| | fluorotetrahydro-1H-pyrrolizin-7a(5H)-yl-5,5-d2)methoxy-d2)pyrido[4,3-d]pyrimidin-7-yl)naphthalen-2-ol; |
| 356 | 5-ethynyl-4-(8-fluoro-4-(((1S,2S)-2-fluorocyclopropyl)(methyl-d3)amino)-2-(((2R,7aS)-2-fluorotetrahydro- |
| | 1H-pyrrolizin-7a(5H)-yl-5,5-d2)methoxy-d2)pyrido[4,3-d]pyrimidin-7-yl)naphthalen-2-ol; |
| 357 | 4-(4-(ethyl((1S,2S)-2-fluorocyclopropyl)amino)-8-fluoro-2-(((2R,7aS)-2-fluorotetrahydro-1H-pyrrolizin- |
| | 7a(5H)-yl-5,5-d2)methoxy-d2)pyrido[4,3-d]pyrimidin-7-yl)-5-ethynylnaphthalen-2-ol; |
| 358 | 5-ethynyl-4-(8-fluoro-4-(((1S,2S)-2-fluorocyclopropyl)(methyl)amino)-2-(((2R,7aS)-2-fluorotetrahydro- |
| | 1H-pyrrolizin-7a(5H)-yl-5,5-d2)methoxy-d2)pyrido[4,3-d]pyrimidin-7-yl)naphthalen-2-ol; |
| 359 | 4-(8-fluoro-4-(((1S,2S)-2-fluorocyclopropyl)(methyl-d3)amino)-2-(((2R,7aS)-2-fluorotetrahydro-1H- |
| | pyrrolizin-7a(5H)-yl)methoxy)pyrido[4,3-d]pyrimidin-7-yl)-5-(methylthio)naphthalen-2-ol; |
| 360 | 4-(4-(ethyl((1S,2S)-2-fluorocyclopropyl)amino)-8-fluoro-2-(((2R,7aS)-2-fluorotetrahydro-1H-pyrrolizin- |
| | 7a(5H)-yl)methoxy)pyrido[4,3-d]pyrimidin-7-yl)-5-(methylthio)naphthalen-2-ol; |
| 361 | 4-(8-fluoro-4-(((1S,2S)-2-fluorocyclopropyl)(methyl)amino)-2-(((2R,7aS)-2-fluorotetrahydro-1H-pyrrolizin- |
| | 7a(5H)-yl)methoxy)pyrido[4,3-d]pyrimidin-7-yl)-5-(methylthio)naphthalen-2-ol; |
| 362 | 5-bromo-6-fluoro-4-(8-fluoro-4-(((1S,2S)-2-fluorocyclopropyl)(methyl-d3)amino)-2-(((2R,7aS)-2- |
| | fluorotetrahydro-1H-pyrrolizin-7a(5H)-yl)methoxy)pyrido[4,3-d]pyrimidin-7-yl)naphthalen-2-ol; |
| 363 | 5-bromo-4-(4-(ethyl((1S,2S)-2-fluorocyclopropyl)amino)-8-fluoro-2-(((2R,7aS)-2-fluorotetrahydro- |
| | 1H-pyrrolizin-7a(5H)-yl)methoxy)pyrido[4,3-d]pyrimidin-7-yl)-6-fluoronaphthalen-2-ol; |
| 364 | 5-bromo-6-fluoro-4-(8-fluoro-4-(((1S,2S)-2-fluorocyclopropyl)(methyl)amino)-2-(((2R,7aS)-2- |
| | fluorotetrahydro-1H-pyrrolizin-7a(5H)-yl)methoxy)pyrido[4,3-d]pyrimidin-7-yl)naphthalen-2-ol; |
| 365 | 5-ethyl-4-(8-fluoro-4-(((1S,2S)-2-fluorocyclopropyl)(methyl-d3)amino)-2-(((2R,7aS)-2-fluorotetrahydro- |
| | 1H-pyrrolizin-7a(5H)-yl)methoxy)pyrido[4,3-d]pyrimidin-7-yl)naphthalen-2-ol; |
| 366 | 5-ethyl-4-(4-(ethyl((1S,2S)-2-fluorocyclopropyl)amino)-8-fluoro-2-(((2R,7aS)-2-fluorotetrahydro- |
| | 1H-pyrrolizin-7a(5H)-yl)methoxy)pyrido[4,3-d]pyrimidin-7-yl)naphthalen-2-ol; |
| 367 | 5-ethyl-4-(8-fluoro-4-(((1S,2S)-2-fluorocyclopropyl)(methyl)amino)-2-(((2R,7aS)-2-fluorotetrahydro- |
| | 1H-pyrrolizin-7a(5H)-yl)methoxy)pyrido[4,3-d]pyrimidin-7-yl)naphthalen-2-ol; |
| 368 | 5,6-difluoro-4-(8-fluoro-4-(((1S,2S)-2-fluorocyclopropyl)(methyl-d3)amino)-2-(((2R,7aS)-2- |
| | fluorotetrahydro-1H-pyrrolizin-7a(5H)-yl)methoxy)pyrido[4,3-d]pyrimidin-7-yl)naphthalen-2-ol; |
| 369 | 4-(4-(ethyl((1S,2S)-2-fluorocyclopropyl)amino)-8-fluoro-2-(((2R,7aS)-2-fluorotetrahydro-1H- |
| | pyrrolizin-7a(5H)-yl)methoxy)pyrido[4,3-d]pyrimidin-7-yl)-5,6-difluoronaphthalen-2-ol; |
| 370 | 5-chloro-6-fluoro-4-(8-fluoro-4-(((1S,2S)-2-fluorocyclopropyl)(methyl-d3)amino)-2-(((2R,7aS)-2- |
| | fluorotetrahydro-1H-pyrrolizin-7a(5H)-yl)methoxy)pyrido[4,3-d]pyrimidin-7-yl)naphthalen-2-ol; |
| 371 | 5-chloro-4-(4-(ethyl((1S,2S)-2-fluorocyclopropyl)amino)-8-fluoro-2-(((2R,7aS)-2-fluorotetrahydro- |
| | 1H-pyrrolizin-7a(5H)-yl)methoxy)pyrido[4,3-d]pyrimidin-7-yl)-6-fluoronaphthalen-2-ol; |
| 372 | 5-chloro-6-fluoro-4-(8-fluoro-4-(((1S,2S)-2-fluorocyclopropyl)(methyl)amino)-2-(((2R,7aS)-2- |
| | fluorotetrahydro-1H-pyrrolizin-7a(5H)-yl)methoxy)pyrido[4,3-d]pyrimidin-7-yl)naphthalen-2-ol; |
| 373 | 5-ethyl-6-fluoro-4-(8-fluoro-4-(((1S,2S)-2-fluorocyclopropyl)(methyl-d3)amino)-2-(((2R,7aS)-2- |
| | fluorotetrahydro-1H-pyrrolizin-7a(5H)-yl)methoxy)pyrido[4,3-d]pyrimidin-7-yl)naphthalen-2-ol; |
| 374 | 5-ethyl-4-(4-(ethyl((1S,2S)-2-fluorocyclopropyl)amino)-8-fluoro-2-(((2R,7aS)-2-fluorotetrahydro- |
| | 1H-pyrrolizin-7a(5H)-yl)methoxy)pyrido[4,3-d]pyrimidin-7-yl)-6-fluoronaphthalen-2-ol; |
| 375 | 5-ethynyl-4-(8-fluoro-4-(((1S,2S)-2-fluorocyclopropyl)(methyl-d3)amino)-2-(((2R,7aS)-2- |
| | fluorotetrahydro-1H-pyrrolizin-7a(5H)-yl)methoxy)pyrido[4,3-d]pyrimidin-7-yl)naphthalen-2-ol; |
| 376 | 4-(4-(ethyl((1S,2S)-2-fluorocyclopropyl)amino)-8-fluoro-2-(((2R,7aS)-2-fluorotetrahydro-1H-pyrrolizin- |
| | 7a(5H)-yl)methoxy)pyrido[4,3-d]pyrimidin-7-yl)-5-ethynylnaphthalen-2-ol; |
| 377 | 5-ethynyl-4-(8-fluoro-4-(((1S,2S)-2-fluorocyclopropyl)(methyl)amino)-2-(((2R,7aS)-2-fluorotetrahydro- |
| | 1H-pyrrolizin-7a(5H)-yl)methoxy)pyrido[4,3-d]pyrimidin-7-yl)naphthalen-2-ol; |
| 378 | 5-ethynyl-6-fluoro-4-(8-fluoro-4-(((1S,2S)-2-fluorocyclopropyl)(methyl-d3)amino)-2-(((2R,7aS)-2- |
| | (methylthio)tetrahydro-1H-pyrrolizin-7a(5H)-yl)methoxy)pyrido[4,3-d]pyrimidin-7-yl)naphthalen-2-ol; |
| 379 | 4-(4-(ethyl((1S,2S)-2-fluorocyclopropyl)amino)-8-fluoro-2-(((2R,7aS)-2-(methylthio)tetrahydro-1H- |
| | pyrrolizin-7a(5H)-yl)methoxy)pyrido[4,3-d]pyrimidin-7-yl)-5-ethynyl-6-fluoronaphthalen-2-ol; |
| 380 | 4-(8-fluoro-4-(((1S,2S)-2-fluorocyclopropyl)(methyl-d3)amino)-2-(((2R,7aS)-2-(methylthio)tetrahydro- |
| | 1H-pyrrolizin-7a(5H)-yl)methoxy)pyrido[4,3-d]pyrimidin-7-yl)-5-(methylthio)naphthalen-2-ol; |
| 381 | 4-(4-(ethyl((1S,2S)-2-fluorocyclopropyl)amino)-8-fluoro-2-(((2R,7aS)-2-(methylthio)tetrahydro-1H- |
| | pyrrolizin-7a(5H)-yl)methoxy)pyrido[4,3-d]pyrimidin-7-yl)-5-(methylthio)naphthalen-2-ol; |
| 382 | 4-(8-fluoro-4-(((1S,2S)-2-fluorocyclopropyl)(methyl)amino)-2-(((2R,7aS)-2-(methylthio)tetrahydro- |
| | 1H-pyrrolizin-7a(5H)-yl)methoxy)pyrido[4,3-d]pyrimidin-7-yl)-5-(methylthio)naphthalen-2-ol; |
| 383 | 5-bromo-6-fluoro-4-(8-fluoro-4-(((1S,2S)-2-fluorocyclopropyl)(methyl-d3)amino)-2-(((2R,7aS)-2- |
| | (methylthio)tetrahydro-1H-pyrrolizin-7a(5H)-yl)methoxy)pyrido[4,3-d]pyrimidin-7-yl)naphthalen-2-ol; |
| 384 | 5-bromo-4-(4-(ethyl((1S,2S)-2-fluorocyclopropyl)amino)-8-fluoro-2-(((2R,7aS)-2-(methylthio)tetrahydro- |
| | 1H-pyrrolizin-7a(5H)-yl)methoxy)pyrido[4,3-d]pyrimidin-7-yl)-6-fluoronaphthalen-2-ol; |
| 385 | 5-bromo-6-fluoro-4-(8-fluoro-4-(((1S,2S)-2-fluorocyclopropyl)(methyl)amino)-2-(((2R,7aS)-2- |
| | (methylthio)tetrahydro-1H-pyrrolizin-7a(5H)-yl)methoxy)pyrido[4,3-d]pyrimidin-7-yl)naphthalen-2-ol; |
| 386 | 5-ethyl-4-(8-fluoro-4-(((1S,2S)-2-fluorocyclopropyl)(methyl-d3)amino)-2-(((2R,7aS)-2- |
| | (methylthio)tetrahydro-1H-pyrrolizin-7a(5H)-yl)methoxy)pyrido[4,3-d]pyrimidin-7-yl)naphthalen-2-ol; |
| 387 | 5-ethyl-4-(4-(ethyl((1S,2S)-2-fluorocyclopropyl)amino)-8-fluoro-2-(((2R,7aS)-2-(methylthio)tetrahydro- |
| | 1H-pyrrolizin-7a(5H)-yl)methoxy)pyrido[4,3-d]pyrimidin-7-yl)naphthalen-2-ol; |
| 388 | 5-ethyl-4-(8-fluoro-4-(((1S,2S)-2-fluorocyclopropyl)(methyl)amino)-2-(((2R,7aS)-2- |
| | (methylthio)tetrahydro-1H-pyrrolizin-7a(5H)-yl)methoxy)pyrido[4,3-d]pyrimidin-7-yl)naphthalen-2-ol; |
| 389 | 5,6-difluoro-4-(8-fluoro-4-(((1S,2S)-2-fluorocyclopropyl)(methyl-d3)amino)-2-(((2R,7aS)-2- |
| | (methylthio)tetrahydro-1H-pyrrolizin-7a(5H)-yl)methoxy)pyrido[4,3-d]pyrimidin-7-yl)naphthalen-2-ol; |
| 390 | 4-(4-(ethyl((1S,2S)-2-fluorocyclopropyl)amino)-8-fluoro-2-(((2R,7aS)-2-(methylthio)tetrahydro-1H- |
| | pyrrolizin-7a(5H)-yl)methoxy)pyrido[4,3-d]pyrimidin-7-yl)-5,6-difluoronaphthalen-2-ol; |
| 391 | 5,6-difluoro-4-(8-fluoro-4-(((1S,2S)-2-fluorocyclopropyl)(methyl)amino)-2-(((2R,7aS)-2- |
| | (methylthio)tetrahydro-1H-pyrrolizin-7a(5H)-yl)methoxy)pyrido[4,3-d]pyrimidin-7-yl)naphthalen-2-ol; |
| 392 | 5-chloro-6-fluoro-4-(8-fluoro-4-(((1S,2S)-2-fluorocyclopropyl)(methyl-d3)amino)-2-(((2R,7aS)-2- |
| | (methylthio)tetrahydro-1H-pyrrolizin-7a(5H)-yl)methoxy)pyrido[4,3-d]pyrimidin-7-yl)naphthalen-2-ol; |
| 393 | 5-chloro-4-(4-(ethyl((1S,2S)-2-fluorocyclopropyl)amino)-8-fluoro-2-(((2R,7aS)-2-(methylthio)tetrahydro- |
| | 1H-pyrrolizin-7a(5H)-yl)methoxy)pyrido[4,3-d]pyrimidin-7-yl)-6-fluoronaphthalen-2-ol; |
| 394 | 5-chloro-6-fluoro-4-(8-fluoro-4-(((1S,2S)-2-fluorocyclopropyl)(methyl)amino)-2-(((2R,7aS)-2- |
| | (methylthio)tetrahydro-1H-pyrrolizin-7a(5H)-yl)methoxy)pyrido[4,3-d]pyrimidin-7-yl)naphthalen-2-ol; |
| 395 | 5-ethyl-6-fluoro-4-(8-fluoro-4-(((1S,2S)-2-fluorocyclopropyl)(methyl-d3)amino)-2-(((2R,7aS)-2- |
| | (methylthio)tetrahydro-1H-pyrrolizin-7a(5H)-yl)methoxy)pyrido[4,3-d]pyrimidin-7-yl)naphthalen-2-ol; |
| 396 | 5-ethyl-4-(4-(ethyl((1S,2S)-2-fluorocyclopropyl)amino)-8-fluoro-2-(((2R,7aS)-2-(methylthio)tetrahydro- |
| | 1H-pyrrolizin-7a(5H)-yl)methoxy)pyrido[4,3-d]pyrimidin-7-yl)-6-fluoronaphthalen-2-ol; |
| 397 | 5-ethyl-6-fluoro-4-(8-fluoro-4-(((1S,2S)-2-fluorocyclopropyl)(methyl)amino)-2-(((2R,7aS)-2- |
| | (methylthio)tetrahydro-1H-pyrrolizin-7a(5H)-yl)methoxy)pyrido[4,3-d]pyrimidin-7-yl)naphthalen-2-ol; |
| 398 | 5-ethynyl-4-(8-fluoro-4-(((1S,2S)-2-fluorocyclopropyl)(methyl-d3)amino)-2-(((2R,7aS)-2- |
| | (methylthio)tetrahydro-1H-pyrrolizin-7a(5H)-yl)methoxy)pyrido[4,3-d]pyrimidin-7-yl)naphthalen-2-ol; |
| 399 | 4-(4-(ethyl((1S,2S)-2-fluorocyclopropyl)amino)-8-fluoro-2-(((2R,7aS)-2-(methylthio)tetrahydro-1H- |
| | pyrrolizin-7a(5H)-yl)methoxy)pyrido[4,3-d]pyrimidin-7-yl)-5-ethynylnaphthalen-2-ol; |
| 400 | 5-ethynyl-4-(8-fluoro-4-(((1S,2S)-2-fluorocyclopropyl)(methyl)amino)-2-(((2R,7aS)-2- |
| | (methylthio)tetrahydro-1H-pyrrolizin-7a(5H)-yl)methoxy)pyrido[4,3-d]pyrimidin-7-yl)naphthalen-2-ol; |
| 401 | 4-(4-(ethyl((1S,2S)-2-fluorocyclopropyl)amino)-8-fluoro-2-(((2R,7aS)-2-fluorotetrahydro-1H-pyrrolizin- |
| | 7a(5H)-yl)methoxy-d2)pyrido[4,3-d]pyrimidin-7-yl)-5-ethynyl-6-fluoronaphthalen-2-ol; |
| 402 | 4-(8-fluoro-4-(((1S,2S)-2-fluorocyclopropyl)(methyl-d3)amino)-2-(((2R,7aS)-2-fluorotetrahydro-1H- |
| | pyrrolizin-7a(5H)-yl)methoxy-d2)pyrido[4,3-d]pyrimidin-7-yl)-5-(methylthio)naphthalen-2-ol; |
| 403 | 4-(4-(ethyl((1S,2S)-2-fluorocyclopropyl)amino)-8-fluoro-2-(((2R,7aS)-2-fluorotetrahydro-1H-pyrrolizin- |
| | 7a(5H)-yl)methoxy-d2)pyrido[4,3-d]pyrimidin-7-yl)-5-(methylthio)naphthalen-2-ol; |
| 404 | 4-(8-fluoro-4-(((1S,2S)-2-fluorocyclopropyl)(methyl)amino)-2-(((2R,7aS)-2-fluorotetrahydro-1H- |
| | pyrrolizin-7a(5H)-yl)methoxy-d2)pyrido[4,3-d]pyrimidin-7-yl)-5-(methylthio)naphthalen-2-ol; |
| 405 | 5-bromo-6-fluoro-4-(8-fluoro-4-(((1S,2S)-2-fluorocyclopropyl)(methyl-d3)amino)-2-(((2R,7aS)-2- |
| | fluorotetrahydro-1H-pyrrolizin-7a(5H)-yl)methoxy-d2)pyrido[4,3-d]pyrimidin-7-yl)naphthalen-2-ol; |
| 406 | 5-bromo-4-(4-(ethyl((1S,2S)-2-fluorocyclopropyl)amino)-8-fluoro-2-(((2R,7aS)-2-fluorotetrahydro- |
| | 1H-pyrrolizin-7a(5H)-yl)methoxy-d2)pyrido[4,3-d]pyrimidin-7-yl)-6-fluoronaphthalen-2-ol; |
| 407 | 5-bromo-6-fluoro-4-(8-fluoro-4-(((1S,2S)-2-fluorocyclopropyl)(methyl)amino)-2-(((2R,7aS)-2- |
| | fluorotetrahydro-1H-pyrrolizin-7a(5H)-yl)methoxy-d2)pyrido[4,3-d]pyrimidin-7-yl)naphthalen-2-ol; |
| 408 | 5-ethyl-4-(8-fluoro-4-(((1S,2S)-2-fluorocyclopropyl)(methyl-d3)amino)-2-(((2R,7aS)-2-fluorotetrahydro- |
| | 1H-pyrrolizin-7a(5H)-yl)methoxy-d2)pyrido[4,3-d]pyrimidin-7-yl)naphthalen-2-ol; |
| 409 | 5-ethyl-4-(4-(ethyl((1S,2S)-2-fluorocyclopropyl)amino)-8-fluoro-2-(((2R,7aS)-2-fluorotetrahydro- |
| | 1H-pyrrolizin-7a(5H)-yl)methoxy-d2)pyrido[4,3-d]pyrimidin-7-yl)naphthalen-2-ol; |
| 410 | 5-ethyl-4-(8-fluoro-4-(((1S,2S)-2-fluorocyclopropyl)(methyl)amino)-2-(((2R,7aS)-2-fluorotetrahydro- |
| | 1H-pyrrolizin-7a(5H)-yl)methoxy-d2)pyrido[4,3-d]pyrimidin-7-yl)naphthalen-2-ol; |
| 411 | 5,6-difluoro-4-(8-fluoro-4-(((1S,2S)-2-fluorocyclopropyl)(methyl-d3)amino)-2-(((2R,7aS)-2- |
| | fluorotetrahydro-1H-pyrrolizin-7a(5H)-yl)methoxy-d2)pyrido[4,3-d]pyrimidin-7-yl)naphthalen-2-ol; |
| 412 | 4-(4-(ethyl((1S,2S)-2-fluorocyclopropyl)amino)-8-fluoro-2-(((2R,7aS)-2-fluorotetrahydro-1H-pyrrolizin- |
| | 7a(5H)-yl)methoxy-d2)pyrido[4,3-d]pyrimidin-7-yl)-5,6-difluoronaphthalen-2-ol; |
| 413 | 5,6-difluoro-4-(8-fluoro-4-(((1S,2S)-2-fluorocyclopropyl)(methyl)amino)-2-(((2R,7aS)-2-fluorotetrahydro- |
| | 1H-pyrrolizin-7a(5H)-yl)methoxy-d2)pyrido[4,3-d]pyrimidin-7-yl)naphthalen-2-ol; |
| 414 | 5-chloro-6-fluoro-4-(8-fluoro-4-(((1S,2S)-2-fluorocyclopropyl)(methyl-d3)amino)-2-(((2R,7aS)-2- |
| | fluorotetrahydro-1H-pyrrolizin-7a(5H)-yl)methoxy-d2)pyrido[4,3-d]pyrimidin-7-yl)naphthalen-2-ol; |
| 415 | 5-chloro-4-(4-(ethyl((1S,2S)-2-fluorocyclopropyl)amino)-8-fluoro-2-(((2R,7aS)-2-fluorotetrahydro- |
| | 1H-pyrrolizin-7a(5H)-yl)methoxy-d2)pyrido[4,3-d]pyrimidin-7-yl)-6-fluoronaphthalen-2-ol; |
| 416 | 5-chloro-6-fluoro-4-(8-fluoro-4-(((1S,2S)-2-fluorocyclopropyl)(methyl)amino)-2-(((2R,7aS)-2- |
| | fluorotetrahydro-1H-pyrrolizin-7a(5H)-yl)methoxy-d2)pyrido[4,3-d]pyrimidin-7-yl)naphthalen-2-ol; |
| 417 | 5-ethyl-6-fluoro-4-(8-fluoro-4-(((1S,2S)-2-fluorocyclopropyl)(methyl-d3)amino)-2-(((2R,7aS)-2- |
| | fluorotetrahydro-1H-pyrrolizin-7a(5H)-yl)methoxy-d2)pyrido[4,3-d]pyrimidin-7-yl)naphthalen-2-ol; |
| 418 | 5-ethyl-4-(4-(ethyl((1S,2S)-2-fluorocyclopropyl)amino)-8-fluoro-2-(((2R,7aS)-2-fluorotetrahydro- |
| | 1H-pyrrolizin-7a(5H)-yl)methoxy-d2)pyrido[4,3-d]pyrimidin-7-yl)-6-fluoronaphthalen-2-ol; |
| 419 | 5-ethyl-6-fluoro-4-(8-fluoro-4-(((1S,2S)-2-fluorocyclopropyl)(methyl)amino)-2-(((2R,7aS)-2-fluorote |
| | trahydro-1H-pyrrolizin-7a(5H)-yl)methoxy-d2)pyrido[4,3-d]pyrimidin-7-yl)naphthalen-2-ol; |
| 420 | 5-ethynyl-4-(8-fluoro-4-(((1S,2S)-2-fluorocyclopropyl)(methyl-d3)amino)-2-(((2R,7aS)-2- |
| | fluorotetrahydro-1H-pyrrolizin-7a(5H)-yl)methoxy-d2)pyrido[4,3-d]pyrimidin-7-yl)naphthalen-2-ol; |
| 421 | 4-(4-(ethyl((1S,2S)-2-fluorocyclopropyl)amino)-8-fluoro-2-(((2R,7aS)-2-fluorotetrahydro-1H-pyrrolizin- |
| | 7a(5H)-yl)methoxy-d2)pyrido[4,3-d]pyrimidin-7-yl)-5-ethynylnaphthalen-2-ol; |
| 422 | 5-ethynyl-4-(8-fluoro-4-(((1S,2S)-2-fluorocyclopropyl)(methyl)amino)-2-(((2R,7aS)-2-fluorotetrahydro- |
| | 1H-pyrrolizin-7a(5H)-yl)methoxy-d2)pyrido[4,3-d]pyrimidin-7-yl)naphthalen-2-ol; |
| 423 | 5-ethynyl-6-fluoro-4-(8-fluoro-4-((2-fluorocyclopropyl)(methyl-d3)amino)-2-(((2R,7aS)-2-fluorotetrahydro- |
| | 1H-pyrrolizin-7a(5H)-yl-2-d)methoxy)pyrido[4,3-d]pyrimidin-7-yl)naphthalen-2-ol; |
| 424 | 4-(4-(ethyl(2-fluorocyclopropyl)amino)-8-fluoro-2-(((2R,7aS)-2-fluorotetrahydro-1H-pyrrolizin- |
| | 7a(5H)-yl-2-d)methoxy)pyrido[4,3-d]pyrimidin-7-yl)-5-ethynyl-6-fluoronaphthalen-2-ol; |
| 425 | 5-ethynyl-6-fluoro-4-(8-fluoro-4-((2-fluorocyclopropyl)(methyl)amino)-2-(((2R,7aS)-2-fluorotetrahydro- |
| | 1H-pyrrolizin-7a(5H)-yl-2-d)methoxy)pyrido[4,3-d]pyrimidin-7-yl)naphthalen-2-ol; |
| 426 | 5-bromo-6-fluoro-4-(8-fluoro-4-((2-fluorocyclopropyl)(methyl-d3)amino)-2-(((2R,7aS)-2-fluorotetrahydro- |
| | 1H-pyrrolizin-7a(5H)-yl-2-d)methoxy)pyrido[4,3-d]pyrimidin-7-yl)naphthalen-2-ol; |
| 427 | 5-bromo-4-(4-(ethyl(2-fluorocyclopropyl)amino)-8-fluoro-2-(((2R,7aS)-2-fluorotetrahydro-1H-pyrrolizin- |
| | 7a(5H)-yl-2-d)methoxy)pyrido[4,3-d]pyrimidin-7-yl)-6-fluoronaphthalen-2-ol; |
| 428 | 5-bromo-6-fluoro-4-(8-fluoro-4-((2-fluorocyclopropyl)(methyl)amino)-2-(((2R,7aS)-2-fluorotetrahydro- |
| | 1H-pyrrolizin-7a(5H)-yl-2-d)methoxy)pyrido[4,3-d]pyrimidin-7-yl)naphthalen-2-ol; |
| 429 | 5-ethyl-4-(8-fluoro-4-((2-fluorocyclopropyl)(methyl-d3)amino)-2-(((2R,7aS)-2-fluorotetrahydro-1H- |
| | pyrrolizin-7a(5H)-yl-2-d)methoxy)pyrido[4,3-d]pyrimidin-7-yl)naphthalen-2-ol; |
| 430 | 5-ethyl-4-(4-(ethyl(2-fluorocyclopropyl)amino)-8-fluoro-2-(((2R,7aS)-2-fluorotetrahydro-1H-pyrrolizin- |
| | 7a(5H)-yl-2-d)methoxy)pyrido[4,3-d]pyrimidin-7-yl)naphthalen-2-ol; |
| 431 | 5-ethyl-4-(8-fluoro-4-((2-fluorocyclopropyl)(methyl)amino)-2-(((2R,7aS)-2-fluorotetrahydro-1H- |
| | pyrrolizin-7a(5H)-yl-2-d)methoxy)pyrido[4,3-d]pyrimidin-7-yl)naphthalen-2-ol; |
| 432 | 5-ethyl-6-fluoro-4-(8-fluoro-4-((2-fluorocyclopropyl)(methyl-d3)amino)-2-(((2R,7aS)-2-fluorotetrahydro- |
| | 1H-pyrrolizin-7a(5H)-yl-2-d)methoxy)pyrido[4,3-d]pyrimidin-7-yl)naphthalen-2-ol; |
| 433 | 5-ethyl-4-(4-(ethyl(2-fluorocyclopropyl)amino)-8-fluoro-2-(((2R,7aS)-2-fluorotetrahydro-1H-pyrrolizin- |
| | 7a(5H)-yl-2-d)methoxy)pyrido[4,3-d]pyrimidin-7-yl)-6-fluoronaphthalen-2-ol; |
| 434 | 5-ethyl-6-fluoro-4-(8-fluoro-4-((2-fluorocyclopropyl)(methyl)amino)-2-(((2R,7aS)-2-fluorotetrahydro- |
| | 1H-pyrrolizin-7a(5H)-yl-2-d)methoxy)pyrido[4,3-d]pyrimidin-7-yl)naphthalen-2-ol; |
| 435 | 5-ethynyl-6-fluoro-4-(8-fluoro-4-((2-fluorocyclopropyl)(methyl-d3)amino)-2-(((2R,7aS)-2- |
| | methoxytetrahydro-1H-pyrrolizin-7a(5H)-yl)methoxy)pyrido[4,3-d]pyrimidin-7-yl)naphthalen-2-ol; |
| 436 | 4-(4-(ethyl(2-fluorocyclopropyl)amino)-8-fluoro-2-(((2R,7aS)-2-methoxytetrahydro-1H-pyrrolizin- |
| | 7a(5H)-yl)methoxy)pyrido[4,3-d]pyrimidin-7-yl)-5-ethynyl-6-fluoronaphthalen-2-ol; |
| 437 | 5-bromo-6-fluoro-4-(8-fluoro-4-((2-fluorocyclopropyl)(methyl-d3)amino)-2-(((2R,7aS)-2- |
| | methoxytetrahydro-1H-pyrrolizin-7a(5H)-yl)methoxy)pyrido[4,3-d]pyrimidin-7-yl)naphthalen-2-ol; |
| 438 | 5-bromo-4-(4-(ethyl(2-fluorocyclopropyl)amino)-8-fluoro-2-(((2R,7aS)-2-methoxytetrahydro-1H- |
| | pyrrolizin-7a(5H)-yl)methoxy)pyrido[4,3-d]pyrimidin-7-yl)-6-fluoronaphthalen-2-ol; |
| 439 | 5-bromo-6-fluoro-4-(8-fluoro-4-((2-fluorocyclopropyl)(methyl)amino)-2-(((2R,7aS)-2-methoxytetrahydro- |
| | 1H-pyrrolizin-7a(5H)-yl)methoxy)pyrido[4,3-d]pyrimidin-7-yl)naphthalen-2-ol; |
| 440 | 5-ethyl-4-(8-fluoro-4-((2-fluorocyclopropyl)(methyl-d3)amino)-2-(((2R,7aS)-2-methoxytetrahydro- |
| | 1H-pyrrolizin-7a(5H)-yl)methoxy)pyrido[4,3-d]pyrimidin-7-yl)naphthalen-2-ol; |
| 441 | 5-ethyl-4-(4-(ethyl(2-fluorocyclopropyl)amino)-8-fluoro-2-(((2R,7aS)-2-methoxytetrahydro-1H- |
| | pyrrolizin-7a(5H)-yl)methoxy)pyrido[4,3-d]pyrimidin-7-yl)naphthalen-2-ol; |
| 442 | 5-ethyl-4-(8-fluoro-4-((2-fluorocyclopropyl)(methyl)amino)-2-(((2R,7aS)-2-methoxytetrahydro-1H- |
| | pyrrolizin-7a(5H)-yl)methoxy)pyrido[4,3-d]pyrimidin-7-yl)naphthalen-2-ol; |
| 443 | 5-ethyl-6-fluoro-4-(8-fluoro-4-((2-fluorocyclopropyl)(methyl-d3)amino)-2-(((2R,7aS)-2-methoxytetrahydro- |
| | 1H-pyrrolizin-7a(5H)-yl)methoxy)pyrido[4,3-d]pyrimidin-7-yl)naphthalen-2-ol; |
| 444 | 5-ethyl-4-(4-(ethyl(2-fluorocyclopropyl)amino)-8-fluoro-2-(((2R,7aS)-2-methoxytetrahydro-1H- |
| | pyrrolizin-7a(5H)-yl)methoxy)pyrido[4,3-d]pyrimidin-7-yl)-6-fluoronaphthalen-2-ol; |
| 445 | 5-ethyl-6-fluoro-4-(8-fluoro-4-((2-fluorocyclopropyl)(methyl)amino)-2-(((2R,7aS)-2-methoxytetrahydro- |
| | 1H-pyrrolizin-7a(5H)-yl)methoxy)pyrido[4,3-d]pyrimidin-7-yl)naphthalen-2-ol; |
| 446 | 5-ethynyl-6-fluoro-4-(8-fluoro-4-((2-fluorocyclopropyl)(methyl-d3)amino)-2-(((2R,7aS)-2-fluorotetrahydro- |
| | 1H-pyrrolizin-7a(5H)-yl-2-d)methoxy-d2)pyrido[4,3-d]pyrimidin-7-yl)naphthalen-2-ol; |
| 447 | 4-(4-(ethyl(2-fluorocyclopropyl)amino)-8-fluoro-2-(((2R,7aS)-2-fluorotetrahydro-1H-pyrrolizin- |
| | 7a(5H)-yl-2-d)methoxy-d2)pyrido[4,3-d]pyrimidin-7-yl)-5-ethynyl-6-fluoronaphthalen-2-ol; |
| 448 | 5-ethynyl-6-fluoro-4-(8-fluoro-4-((2-fluorocyclopropyl)(methyl)amino)-2-(((2R,7aS)-2-fluorotetrahydro- |
| | 1H-pyrrolizin-7a(5H)-yl-2-d)methoxy-d2)pyrido[4,3-d]pyrimidin-7-yl)naphthalen-2-ol; |
| 449 | 5-bromo-6-fluoro-4-(8-fluoro-4-((2-fluorocyclopropyl)(methyl-d3)amino)-2-(((2R,7aS)-2-fluorotetrahydro- |
| | 1H-pyrrolizin-7a(5H)-yl-2-d)methoxy-d2)pyrido[4,3-d]pyrimidin-7-yl)naphthalen-2-ol; |
| 450 | 5-bromo-4-(4-(ethyl(2-fluorocyclopropyl)amino)-8-fluoro-2-(((2R,7aS)-2-fluorotetrahydro-1H- |
| | pyrrolizin-7a(5H)-yl-2-d)methoxy-d2)pyrido[4,3-d]pyrimidin-7-yl)-6-fluoronaphthalen-2-ol; |
| 451 | 5-bromo-6-fluoro-4-(8-fluoro-4-((2-fluorocyclopropyl)(methyl)amino)-2-(((2R,7aS)-2-fluorotetrahydro- |
| | 1H-pyrrolizin-7a(5H)-yl-2-d)methoxy-d2)pyrido[4,3-d]pyrimidin-7-yl)naphthalen-2-ol; |
| 452 | 5-ethyl-4-(8-fluoro-4-((2-fluorocyclopropyl)(methyl-d3)amino)-2-(((2R,7aS)-2-fluorotetrahydro-1H- |
| | pyrrolizin-7a(5H)-yl-2-d)methoxy-d2)pyrido[4,3-d]pyrimidin-7-yl)naphthalen-2-ol; |
| 453 | 5-ethyl-4-(4-(ethyl(2-fluorocyclopropyl)amino)-8-fluoro-2-(((2R,7aS)-2-fluorotetrahydro-1H- |
| | pyrrolizin-7a(5H)-yl-2-d)methoxy-d2)pyrido[4,3-d]pyrimidin-7-yl)naphthalen-2-ol; |
| 454 | 5-ethyl-4-(8-fluoro-4-((2-fluorocyclopropyl)(methyl)amino)-2-(((2R,7aS)-2-fluorotetrahydro-1H- |
| | pyrrolizin-7a(5H)-yl-2-d)methoxy-d2)pyrido[4,3-d]pyrimidin-7-yl)naphthalen-2-ol; |
| 455 | 5-ethyl-6-fluoro-4-(8-fluoro-4-((2-fluorocyclopropyl)(methyl-d3)amino)-2-(((2R,7aS)-2-fluorotetrahydro- |
| | 1H-pyrrolizin-7a(5H)-yl-2-d)methoxy-d2)pyrido[4,3-d]pyrimidin-7-yl)naphthalen-2-ol; |
| 456 | 5-ethyl-4-(4-(ethyl(2-fluorocyclopropyl)amino)-8-fluoro-2-(((2R,7aS)-2-fluorotetrahydro-1H-pyrrolizin- |
| | 7a(5H)-yl-2-d)methoxy-d2)pyrido[4,3-d]pyrimidin-7-yl)-6-fluoronaphthalen-2-ol; |
| 457 | 5-ethyl-6-fluoro-4-(8-fluoro-4-((2-fluorocyclopropyl)(methyl)amino)-2-(((2R,7aS)-2-fluorotetrahydro- |
| | 1H-pyrrolizin-7a(5H)-yl-2-d)methoxy-d2)pyrido[4,3-d]pyrimidin-7-yl)naphthalen-2-ol; |
| 458 | 5-bromo-6-fluoro-4-(8-fluoro-4-((2-fluorocyclopropyl)(methyl-d3)amino)-2-(((2R,7aS)-2-fluorotetrahydro- |
| | 1H-pyrrolizin-7a(5H)-yl)methoxy)pyrido[4,3-d]pyrimidin-7-yl)naphthalen-2-ol; |
| 459 | 5-bromo-4-(4-(ethyl(2-fluorocyclopropyl)amino)-8-fluoro-2-(((2R,7aS)-2-fluorotetrahydro-1H-pyrrolizin- |
| | 7a(5H)-yl)methoxy)pyrido[4,3-d]pyrimidin-7-yl)-6-fluoronaphthalen-2-ol; |
| 460 | 5-bromo-6-fluoro-4-(8-fluoro-4-((2-fluorocyclopropyl)(methyl)amino)-2-(((2R,7aS)-2-fluorotetrahydro- |
| | 1H-pyrrolizin-7a(5H)-yl)methoxy)pyrido[4,3-d]pyrimidin-7-yl)naphthalen-2-ol; |
| 461 | 5-ethyl-4-(8-fluoro-4-((2-fluorocyclopropyl)(methyl-d3)amino)-2-(((2R,7aS)-2-fluorotetrahydro-1H- |
| | pyrrolizin-7a(5H)-yl)methoxy)pyrido[4,3-d]pyrimidin-7-yl)naphthalen-2-ol; |
| 462 | 5-ethyl-4-(4-(ethyl(2-fluorocyclopropyl)amino)-8-fluoro-2-(((2R,7aS)-2-fluorotetrahydro-1H-pyrrolizin- |
| | 7a(5H)-yl)methoxy)pyrido[4,3-d]pyrimidin-7-yl)naphthalen-2-ol; |
| 463 | 5-ethyl-4-(8-fluoro-4-((2-fluorocyclopropyl)(methyl)amino)-2-(((2R,7aS)-2-fluorotetrahydro-1H- |
| | pyrrolizin-7a(5H)-yl)methoxy)pyrido[4,3-d]pyrimidin-7-yl)naphthalen-2-ol; |
| 464 | 5-ethyl-6-fluoro-4-(8-fluoro-4-((2-fluorocyclopropyl)(methyl-d3)amino)-2-(((2R,7aS)-2-fluorotetrahydro- |
| | 1H-pyrrolizin-7a(5H)-yl)methoxy)pyrido[4,3-d]pyrimidin-7-yl)naphthalen-2-ol; |
| 465 | 5-ethyl-4-(4-(ethyl(2-fluorocyclopropyl)amino)-8-fluoro-2-(((2R,7aS)-2-fluorotetrahydro-1H-pyrrolizin- |
| | 7a(5H)-yl)methoxy)pyrido[4,3-d]pyrimidin-7-yl)-6-fluoronaphthalen-2-ol; |
| 466 | 5-ethynyl-6-fluoro-4-(8-fluoro-4-((2-fluorocyclopropyl)(methyl-d3)amino)-2-(((2R,7aS)-2- |
| | (methylthio)tetrahydro-1H-pyrrolizin-7a(5H)-yl)methoxy)pyrido[4,3-d]pyrimidin-7-yl)naphthalen-2-ol; |
| 467 | 4-(4-(ethyl(2-fluorocyclopropyl)amino)-8-fluoro-2-(((2R,7aS)-2-(methylthio)tetrahydro-1H- |
| | pyrrolizin-7a(5H)-yl)methoxy)pyrido[4,3-d]pyrimidin-7-yl)-5-ethynyl-6-fluoronaphthalen-2-ol; |
| 468 | 5-ethynyl-6-fluoro-4-(8-fluoro-4-((2-fluorocyclopropyl)(methyl)amino)-2-(((2R,7aS)-2- |
| | (methylthio)tetrahydro-1H-pyrrolizin-7a(5H)-yl)methoxy)pyrido[4,3-d]pyrimidin-7-yl)naphthalen-2-ol; |
| 469 | 5-bromo-6-fluoro-4-(8-fluoro-4-((2-fluorocyclopropyl)(methyl-d3)amino)-2-(((2R,7aS)-2- |
| | (methylthio)tetrahydro-1H-pyrrolizin-7a(5H)-yl)methoxy)pyrido[4,3-d]pyrimidin-7-yl)naphthalen-2-ol; |
| 470 | 5-bromo-4-(4-(ethyl(2-fluorocyclopropyl)amino)-8-fluoro-2-(((2R,7aS)-2-(methylthio)tetrahydro-1H- |
| | pyrrolizin-7a(5H)-yl)methoxy)pyrido[4,3-d]pyrimidin-7-yl)-6-fluoronaphthalen-2-ol; |
| 471 | 5-bromo-6-fluoro-4-(8-fluoro-4-((2-fluorocyclopropyl)(methyl)amino)-2-(((2R,7aS)-2- |
| | (methylthio)tetrahydro-1H-pyrrolizin-7a(5H)-yl)methoxy)pyrido[4,3-d]pyrimidin-7-yl)naphthalen-2-ol; |
| 472 | 5-ethyl-4-(8-fluoro-4-((2-fluorocyclopropyl)(methyl-d3)amino)-2-(((2R,7aS)-2-(methylthio)tetrahydro- |
| | 1H-pyrrolizin-7a(5H)-yl)methoxy)pyrido[4,3-d]pyrimidin-7-yl)naphthalen-2-ol; |
| 473 | 5-ethyl-4-(4-(ethyl(2-fluorocyclopropyl)amino)-8-fluoro-2-(((2R,7aS)-2-(methylthio)tetrahydro-1H- |
| | pyrrolizin-7a(5H)-yl)methoxy)pyrido[4,3-d]pyrimidin-7-yl)naphthalen-2-ol; |
| 474 | 5-ethyl-4-(8-fluoro-4-((2-fluorocyclopropyl)(methyl)amino)-2-(((2R,7aS)-2-(methylthio)tetrahydro- |
| | 1H-pyrrolizin-7a(5H)-yl)methoxy)pyrido[4,3-d]pyrimidin-7-yl)naphthalen-2-ol; |
| 475 | 5-ethyl-6-fluoro-4-(8-fluoro-4-((2-fluorocyclopropyl)(methyl-d3)amino)-2-(((2R,7aS)-2-(methylthio)tetrahydro- |
| | 1H-pyrrolizin-7a(5H)-yl)methoxy)pyrido[4,3-d]pyrimidin-7-yl)naphthalen-2-ol; |
| 476 | 5-ethyl-4-(4-(ethyl(2-fluorocyclopropyl)amino)-8-fluoro-2-(((2R,7aS)-2-(methylthio)tetrahydro-1H- |
| | pyrrolizin-7a(5H)-yl)methoxy)pyrido[4,3-d]pyrimidin-7-yl)-6-fluoronaphthalen-2-ol; |
| 477 | 5-ethyl-6-fluoro-4-(8-fluoro-4-((2-fluorocyclopropyl)(methyl)amino)-2-(((2R,7aS)-2-(methylthio)tetrahydro- |
| | 1H-pyrrolizin-7a(5H)-yl)methoxy)pyrido[4,3-d]pyrimidin-7-yl)naphthalen-2-ol; |
| 478 | 5-ethynyl-6-fluoro-4-(8-fluoro-4-((2-fluorocyclopropyl)(methyl-d3)amino)-2-(((2R,7aS)-2-fluorotetrahydro- |
| | 1H-pyrrolizin-7a(5H)-yl)methoxy-d2)pyrido[4,3-d]pyrimidin-7-yl)naphthalen-2-ol; |
| 479 | 4-(4-(ethyl(2-fluorocyclopropyl)amino)-8-fluoro-2-(((2R,7aS)-2-fluorotetrahydro-1H-pyrrolizin- |
| | 7a(5H)-yl)methoxy-d2)pyrido[4,3-d]pyrimidin-7-yl)-5-ethynyl-6-fluoronaphthalen-2-ol; |
| 480 | 5-ethynyl-6-fluoro-4-(8-fluoro-4-((2-fluorocyclopropyl)(methyl)amino)-2-(((2R,7aS)-2-fluorotetrahydro- |
| | 1H-pyrrolizin-7a(5H)-yl)methoxy-d2)pyrido[4,3-d]pyrimidin-7-yl)naphthalen-2-ol; |
| 481 | 5-bromo-6-fluoro-4-(8-fluoro-4-((2-fluorocyclopropyl)(methyl-d3)amino)-2-(((2R,7aS)-2-fluorotetrahydro- |
| | 1H-pyrrolizin-7a(5H)-yl)methoxy-d2)pyrido[4,3-d]pyrimidin-7-yl)naphthalen-2-ol; |
| 482 | 5-bromo-4-(4-(ethyl(2-fluorocyclopropyl)amino)-8-fluoro-2-(((2R,7aS)-2-fluorotetrahydro-1H-pyrrolizin- |
| | 7a(5H)-yl)methoxy-d2)pyrido[4,3-d]pyrimidin-7-yl)-6-fluoronaphthalen-2-ol; |
| 483 | 5-bromo-6-fluoro-4-(8-fluoro-4-((2-fluorocyclopropyl)(methyl)amino)-2-(((2R,7aS)-2-fluorotetrahydro- |
| | 1H-pyrrolizin-7a(5H)-yl)methoxy-d2)pyrido[4,3-d]pyrimidin-7-yl)naphthalen-2-ol; |
| 484 | 5-ethyl-4-(8-fluoro-4-((2-fluorocyclopropyl)(methyl-d3)amino)-2-(((2R,7aS)-2-fluorotetrahydro-1H- |
| | pyrrolizin-7a(5H)-yl)methoxy-d2)pyrido[4,3-d]pyrimidin-7-yl)naphthalen-2-ol; |
| 485 | 5-ethyl-4-(4-(ethyl(2-fluorocyclopropyl)amino)-8-fluoro-2-(((2R,7aS)-2-fluorotetrahydro-1H- |
| | pyrrolizin-7a(5H)-yl)methoxy-d2)pyrido[4,3-d]pyrimidin-7-yl)naphthalen-2-ol; |
| 486 | 5-ethyl-4-(8-fluoro-4-((2-fluorocyclopropyl)(methyl)amino)-2-(((2R,7aS)-2-fluorotetrahydro-1H- |
| | pyrrolizin-7a(5H)-yl)methoxy-d2)pyrido[4,3-d]pyrimidin-7-yl)naphthalen-2-ol; |
| 487 | 5-ethyl-6-fluoro-4-(8-fluoro-4-((2-fluorocyclopropyl)(methyl-d3)amino)-2-(((2R,7aS)-2-fluorotetrahydro- |
| | 1H-pyrrolizin-7a(5H)-yl)methoxy-d2)pyrido[4,3-d]pyrimidin-7-yl)naphthalen-2-ol; |
| 488 | 5-ethyl-4-(4-(ethyl(2-fluorocyclopropyl)amino)-8-fluoro-2-(((2R,7aS)-2-fluorotetrahydro-1H-pyrrolizin- |
| | 7a(5H)-yl)methoxy-d2)pyrido[4,3-d]pyrimidin-7-yl)-6-fluoronaphthalen-2-ol; |
| 489 | 5-ethyl-6-fluoro-4-(8-fluoro-4-((2-fluorocyclopropyl)(methyl)amino)-2-(((2R,7aS)-2-fluorotetrahydro- |
| | 1H-pyrrolizin-7a(5H)-yl)methoxy-d2)pyrido[4,3-d]pyrimidin-7-yl)naphthalen-2-ol; |
| 490 | 5-ethynyl-6-fluoro-4-(8-fluoro-4-(((1S,2S)-2-fluorocyclopropyl)(methyl)amino)-2-(((2R,7aS)-2- |
| | fluorotetrahydro-1H-pyrrolizin-7a(5H)-yl)methoxy)pyrido[4,3-d]pyrimidin-7-yl)naphthalen-2-yl |
| | methylcarbamate; |
| 491 | 5-ethynyl-6-fluoro-4-(8-fluoro-4-(((1S,2S)-2-fluorocyclopropyl)(methyl)amino)-2-(((2R,7aS)-2- |
| | fluorotetrahydro-1H-pyrrolizin-7a(5H)-yl)methoxy)pyrido[4,3-d]pyrimidin-7-yl)naphthalen-2-yl |
| | (tetrahydro-2H-pyran-4-yl)carbamate; |
| 492 | 7-(8-ethynyl-7-fluoro-3-(methoxymethoxy)naphthalen-1-yl)-8-fluoro-N-((1S,2S)-2-fluorocyclopropyl)-2- |
| | (((2R,7aS)-2-fluorotetrahydro-1H-pyrrolizin-7a(5H)-yl)methoxy)-N-methylpyrido[4,3-d]pyrimidin-4-amine; |
| 493 | 7-(8-ethynyl-7-fluoro-3-(isopropoxymethoxy)naphthalen-1-yl)-8-fluoro-N-((1S,2S)-2-fluorocyclopropyl)-2- |
| | (((2R,7aS)-2-fluorotetrahydro-1H-pyrrolizin-7a(5H)-yl)methoxy)-N-methylpyrido[4,3-d]pyrimidin-4-amine; |
| 494 | 7-(8-ethynyl-7-fluoro-3-((2-methoxyethoxy)methoxy)naphthalen-1-yl)-8-fluoro-N-((1S,2S)-2- |
| | fluorocyclopropyl)-2-(((2R,7aS)-2-fluorotetrahydro-1H-pyrrolizin-7a(5H)-yl)methoxy)-N- |
| | methylpyrido[4,3-d]pyrimidin-4-amine; |
| 495 | 5-ethynyl-6-fluoro-4-(8-fluoro-4-(((1S,2S)-2-fluorocyclopropyl)(methyl)amino)-2-(((2R,7aS)-2- |
| | fluorotetrahydro-1H-pyrrolizin-7a(5H)-yl)methoxy)pyrido[4,3-d]pyrimidin-7-yl)naphthalen-2-yl |
| | decanoate; |
| 496 | 5-ethynyl-6-fluoro-4-(8-fluoro-4-(((1S,2S)-2-fluorocyclopropyl)(methyl)amino)-2-(((2R,7aS)-2- |
| | fluorotetrahydro-1H-pyrrolizin-7a(5H)-yl)methoxy)pyrido[4,3-d]pyrimidin-7-yl)naphthalen-2-yl |
| | (2-methoxyethyl)carbamate; |
| 497 | 5-ethynyl-6-fluoro-4-(8-fluoro-4-(((1S,2S)-2-fluorocyclopropyl)(methyl)amino)-2-(((2R,7aS)-2- |
| | fluorotetrahydro-1H-pyrrolizin-7a(5H)-yl)methoxy)pyrido[4,3-d]pyrimidin-7-yl)naphthalen-2-yl |
| | (((S)-tetrahydrofuran-2-yl)methyl)carbamate; |
| 498 | isopropyl |
| | (ethoxy((5-ethynyl-6-fluoro-4-(8-fluoro-4-(((1S,2S)-2-fluorocyclopropyl)(methyl)amino)-2-(((2R,7aS)- |
| | 2-fluorotetrahydro-1H-pyrrolizin-7a(5H)-yl)methoxy)pyrido[4,3-d]pyrimidin-7-yl)naphthalen-2- |
| | yl)oxy)phosphoryl)-L-alaninate; |
| 499 | 5-ethynyl-6-fluoro-4-(8-fluoro-4-(((1S,2S)-2-fluorocyclopropyl)(methyl)amino)-2-(((2R,7aS)-2- |
| | fluorotetrahydro-1H-pyrrolizin-7a(5H)-yl)methoxy)pyrido[4,3-d]pyrimidin-7-yl)naphthalen-2-yl |
| | methyl(pyridin-2-yl)carbamate; |
| 500 | 5-ethynyl-6-fluoro-4-(8-fluoro-4-(((1S,2S)-2-fluorocyclopropyl)(methyl)amino)-2-(((2R,7aS)-2- |
| | fluorotetrahydro-1H-pyrrolizin-7a(5H)-yl)methoxy)pyrido[4,3-d]pyrimidin-7-yl)naphthalen-2-yl |
| | pyridin-2-ylcarbamate; |
| 501 | 5-ethynyl-6-fluoro-4-(8-fluoro-4-(((1S,2S)-2-fluorocyclopropyl)(methyl)amino)-2-(((2R,7aS)-2- |
| | fluorotetrahydro-1H-pyrrolizin-7a(5H)-yl)methoxy)pyrido[4,3-d]pyrimidin-7-yl)naphthalen-2-yl |
| | 4-nitrobenzenesulfonate; |
| 502 | 5-ethynyl-6-fluoro-4-(8-fluoro-4-(((1S,2S)-2-fluorocyclopropyl)(methyl)amino)-2-(((2R,7aS)-2- |
| | fluorotetrahydro-1H-pyrrolizin-7a(5H)-yl)methoxy)pyrido[4,3-d]pyrimidin-7-yl)naphthalen-2-yl |
| | isopropylcarbamate; |
| 503 | 5-ethynyl-6-fluoro-4-(8-fluoro-4-(((1S,2S)-2-fluorocyclopropyl)(methyl)amino)-2-(((2R,7aS)-2- |
| | fluorotetrahydro-1H-pyrrolizin-7a(5H)-yl)methoxy)pyrido[4,3-d]pyrimidin-7-yl)naphthalen-2-yl |
| | adamantane-1-carboxylate; |
| 504 | 5-ethynyl-6-fluoro-4-(8-fluoro-4-(((1S,2S)-2-fluorocyclopropyl)(methyl)amino)-2-(((2R,7aS)-2- |
| | fluorotetrahydro-1H-pyrrolizin-7a(5H)-yl)methoxy)pyrido[4,3-d]pyrimidin-7-yl)naphthalen-2-yl |
| | 2-amino-6-methylbenzoate; |
| 505 | 5-ethynyl-6-fluoro-4-(8-fluoro-4-(((1S,2S)-2-fluorocyclopropyl)(methyl)amino)-2-(((2R,7aS)-2- |
| | fluorotetrahydro-1H-pyrrolizin-7a(5H)-yl)methoxy)pyrido[4,3-d]pyrimidin-7-yl)naphthalen-2-yl |
| | 2-(dimethylamino)-2-phenylacetate; |
| 506 | 5-ethynyl-6-fluoro-4-(8-fluoro-4-(((1S,2S)-2-fluorocyclopropyl-3,3-d2)(methyl)amino)-2-(((2R,7aS)- |
| | 2-fluorotetrahydro-1H-pyrrolizin-7a(5H)-yl)methoxy)pyrido[4,3-d]pyrimidin-7-yl)naphthalen-2-ol; |
| 507 | 5-ethynyl-6-fluoro-4-(8-fluoro-4-(((1S,2S)-2-fluorocyclopropyl-2-d)(methyl)amino)-2-(((2R,7aS)-2- |
| | fluorotetrahydro-1H-pyrrolizin-7a(5H)-yl)methoxy)pyrido[4,3-d]pyrimidin-7-yl)naphthalen-2-ol; |
| 508 | 5-ethynyl-6-fluoro-4-(8-fluoro-4-(((1S,2S)-2-fluorocyclopropyl-1-d)(methyl)amino)-2-(((2R,7aS)-2- |
| | fluorotetrahydro-1H-pyrrolizin-7a(5H)-yl)methoxy)pyrido[4,3-d]pyrimidin-7-yl)naphthalen-2-ol; |
| 509 | 5-ethynyl-6-fluoro-4-(8-fluoro-4-(((1S,2S)-2-fluorocyclopropyl)(methyl)amino)-2-(((2R,7aS)-2- |
| | fluorotetrahydro-1H-pyrrolizin-7a(5H)-yl)methoxy)pyrido[4,3-d]pyrimidin-7-yl)naphthalen-2-yl sulfamate |
| | 2,2,2-trifluoroacetic acid; |
| 510 | ethyl2-((5-ethynyl-6-fluoro-4-(8-fluoro-4-(((1S,2S)-2-fluorocyclopropyl)(methyl)amino)-2-(((2R,7aS)- |
| | 2-fluorotetrahydro-1H-pyrrolizin-7a(5H)-yl)methoxy)pyrido[4,3-d]pyrimidin-7-yl)naphthalen-2- |
| | yl)oxy)acetate 2,2,2-trifluoroacetic acid; |
| 511 | 5-ethynyl-6-fluoro-4-(8-fluoro-4-(((1S,2S)-2-fluorocyclopropyl)(methyl)amino)-2-(((2R,7aS)-2- |
| | fluorotetrahydro-1H-pyrrolizin-7a(5H)-yl)methoxy)pyrido[4,3-d]pyrimidin-7-yl)naphthalen-2-yl |
| | (2-acetamidoethyl)(methyl)carbamate 2,2,2-trifluoroacetic acid; |
| 512 | 5-ethynyl-6-fluoro-4-(8-fluoro-4-(((1S,2S)-2-fluorocyclopropyl)(methyl)amino)-2-(((2R,7aS)-2- |
| | fluorotetrahydro-1H-pyrrolizin-7a(5H)-yl)methoxy)pyrido[4,3-d]pyrimidin-7-yl)naphthalen-2-yl |
| | methyl(2-(2,2,2-trifluoroacetamido)ethyl)carbamate 2,2,2-trifluoroacetic acid; |
| 513 | 1-(((2-((((5-ethynyl-6-fluoro-4-(8-fluoro-4-(((1S,2S)-2-fluorocyclopropyl)(methyl)amino)-2-(((2R,7aS)- |
| | 2-fluorotetrahydro-1H-pyrrolizin-7a(5H)-yl)methoxy)pyrido[4,3-d]pyrimidin-7-yl)naphthalen-2- |
| | yl)oxy)carbonyl)(methyl)amino)ethyl)carbamoyl)oxy)ethyl isobutyrate 2,2,2-trifluoroacetic acid; |
| 514 | 5-ethynyl-6-fluoro-4-(8-fluoro-4-(((1S,2S)-2-fluorocyclopropyl)(methyl)amino)-2-((2-fluorotetrahydro- |
| | 1H-pyrrolizin-7a(5H)-yl)methoxy-d2)pyrido[4,3-d]pyrimidin-7-yl)naphthalen-2-ol |
| | 2,2,2-trifluoroacetic acid; |
| 515 | 5-ethynyl-6-fluoro-4-(8-fluoro-2-(((2R,7aS)-2-fluorotetrahydro-1H-pyrrolizin-7a(5H)-yl)methoxy)-4- |
| | ((2-methoxycyclopropyl)(methyl)amino)pyrido[4,3-d]pyrimidin-7-yl)naphthalen-2-ol |
| | 2,2,2-trifluoroacetic acid; |
| 516 | 4-(4-((2-(benzyloxy)cyclopropyl)(methyl)amino)-8-fluoro-2-(((2R,7aS)-2-fluorotetrahydro-1H-pyrrolizin- |
| | 7a(5H)-yl)methoxy)pyrido[4,3-d]pyrimidin-7-yl)-5-ethynyl-6-fluoronaphthalen-2-ol; |
| 517 | 5-ethynyl-6-fluoro-4-(8-fluoro-2-(((2R,7aS)-2-fluorotetrahydro-1H-pyrrolizin-7a(5H)-yl)methoxy)-4- |
| | (methyl(spiro[2.2]pentan-1-yl)amino)pyrido[4,3-d]pyrimidin-7-yl)naphthalen-2-ol; |
| 518 | 5-ethynyl-6-fluoro-4-(8-fluoro-2-(((2R,7aS)-2-fluorotetrahydro-1H-pyrrolizin-7a(5H)-yl)methoxy)-4- |
| | (methyl(spiro[2.3]hexan-1-yl)amino)pyrido[4,3-d]pyrimidin-7-yl)naphthalen-2-ol; |
| 519 | 4-(4-((3-oxabicyclo[3.1.0]hexan-6-yl)amino)-8-fluoro-2-(((2R,7aS)-2-fluorotetrahydro-1H-pyrrolizin- |
| | 7a(5H)-yl)methoxy)pyrido[4,3-d]pyrimidin-7-yl)-5-ethynyl-6-fluoronaphthalen-2-ol; |
| 520 | 4-(4-((3-oxabicyclo[3.1.0]hexan-6-yl)(methyl)amino)-8-fluoro-2-(((2R,7aS)-2-fluorotetrahydro-1H- |
| | pyrrolizin-7a(5H)-yl)methoxy)pyrido[4,3-d]pyrimidin-7-yl)-5-ethynyl-6-fluoronaphthalen-2-ol; |
| 521 | 4-(4-(bicyclo[4.1.0]heptan-7-ylamino)-8-fluoro-2-(((2R,7aS)-2-fluorotetrahydro-1H-pyrrolizin-7a(5H)- |
| | yl)methoxy)pyrido[4,3-d]pyrimidin-7-yl)-5-ethynyl-6-fluoronaphthalen-2-ol; |
| 522 | 4-(4-(bicyclo[4.1.0]heptan-7-yl(methyl)amino)-8-fluoro-2-(((2R,7aS)-2-fluorotetrahydro-1H-pyrrolizin- |
| | 7a(5H)-yl)methoxy)pyrido[4,3-d]pyrimidin-7-yl)-5-ethynyl-6-fluoronaphthalen-2-ol; |
| 523 | 5-ethynyl-6-fluoro-4-(8-fluoro-2-(((2R,7aS)-2-fluorotetrahydro-1H-pyrrolizin-7a(5H)-yl)methoxy)-4- |
| | ((1-(trifluoromethyl)cyclopropyl)amino)pyrido[4,3-d]pyrimidin-7-yl)naphthalen-2-ol; |
| 524 | 5-ethynyl-6-fluoro-4-(8-fluoro-2-(((2R,7aS)-2-fluorotetrahydro-1H-pyrrolizin-7a(5H)-yl)methoxy)-4- |
| | ((1-(methoxymethyl)cyclopropyl)amino)pyrido[4,3-d]pyrimidin-7-yl)naphthalen-2-ol; or |
| 525 | 1-((7-(8-ethynyl-7-fluoro-3-hydroxynaphthalen-1-yl)-8-fluoro-2-(((2R,7aS)-2-fluorotetrahydro-1H- |
| | pyrrolizin-7a(5H)-yl)methoxy)pyrido[4,3-d]pyrimidin-4-yl)(methyl)amino)cyclopropane-1-carbonitrile. |

[37]. A pharmaceutical composition, comprising a therapeutically effective amount of the compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of any one of [1] to [36], and a pharmaceutically acceptable excipient.

[38]. A method for treating cancer in a subject comprising administering a therapeutically effective amount of the compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of any one of [1] to [36], or the pharmaceutical composition of [37] to a subject in need thereof.

[39]. A method for treating cancer in a subject in need thereof, the method comprising:
(a) determining whether the cancer is associated with K-Ras G12C, K-Ras G12D, K-Ras G12V, K-Ras G13D, K-Ras G12R, K-Ras G12S, K-Ras G12A, K-Ras Q61H mutation and/or K-Ras wild type amplification; and
(b) if so, administering a therapeutically effective amount of the compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of any one of [1] to [36], or the pharmaceutical composition of [37] to the subject in need thereof.

[40]. The compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of any one of [1] to [36], or the pharmaceutical composition of [37] for use in therapy.

[41]. The compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of any one of [1] to [36], or the pharmaceutical composition of [37] for use as a medicament.

[42]. The compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of any one of [1] to [36], or the pharmaceutical composition of [37] for use in a method for the treatment of cancer.

[43]. A use of the compound of Formula (IB), a stereoisomer thereof, a pharmaceutically acceptable salt thereof, a pharmaceutically acceptable salt of the stereoisomer thereof, a prodrug thereof, a deuterated molecule thereof or a conjugated form thereof of any one of [1] to [36], or the pharmaceutical composition of [37] for the treatment of cancer.

[44]. A use of the compound of Formula (IB), a stereoisomer thereof, a pharmaceutically acceptable salt thereof, a pharmaceutically acceptable salt of the stereoisomer thereof, a prodrug thereof, a deuterated molecule thereof or a conjugated form thereof of any one of [1] to [36], or the pharmaceutical composition of [37] for the manufacture of a medicament for the treatment of cancer.

[45]. The method for treating cancer of [38], the use in a method for the treatment of cancer of [42], the use for the treatment of cancer of [43], or the use for the manufacture of a medicament for the treatment of cancer of [44], wherein, said cancer is selected from pancreatic carcinoma, colorectal carcinoma, lung carcinoma (such as non-small cell lung cancer), breast carcinoma, large intestine carcinoma, stomach carcinoma, endometrial carcinoma, esophageal carcinoma or gastroesophageal junction carcinoma.

[46]. The method for treating cancer of [38] or [45], the use in a method for the treatment of cancer of [42] or [45], the use for the treatment of cancer of [43] or [45], or the use for the manufacture of a medicament for the treatment of cancer of [44] or [45], wherein, the cancer is associated with at least one of K-Ras G12C, K-Ras G12D, K-Ras G12V, K-Ras G13D, K-Ras G12R, K-Ras G12S, K-Ras G12A, K-Ras Q61H mutation and/or K-Ras wild type amplification.

[47]. The method for treating cancer of [38], [45] or [46], the use in a method for the treatment of cancer of [42], [45] or [46], the use for the treatment of cancer of [43], [45] or [46], or the use for the manufacture of a medicament for the treatment of cancer of [44], [45] or [46], wherein, the cancer a K-Ras G12C associated cancer.

[48]. The method for treating cancer of [38], [45] or [46], the use in a method for the treatment of cancer of [42], [45] or [46], the use for the treatment of cancer of [43], [45] or [46], or the use for the manufacture of a medicament for the treatment of cancer of [44], [45] or [46], wherein, the cancer a K-Ras G12D associated cancer.

[49]. The method for treating cancer of [38], [45] or [46], the use in a method for the treatment of cancer of [42], [45] or [46], the use for the treatment of cancer of [43], [45] or [46], or the use for the manufacture of a medicament for the treatment of cancer of [44], [45] or [46], wherein, the cancer a K-Ras G12V associated cancer.

[50]. The method for treating cancer of [38], [45] or [46], the use in a method for the treatment of cancer of [42], [45] or [46], the use for the treatment of cancer of [43], [45] or [46], or the use for the manufacture of a medicament for the treatment of cancer of [44], [45] or [46], wherein, the cancer a K-Ras G13D associated cancer.

[51]. The method for treating cancer of [38], [45] or [46], the use in a method for the treatment of cancer of [42], [45] or [46], the use for the treatment of cancer of [43], [45] or [46], or the use for the manufacture of a medicament for the treatment of cancer of [44], [45] or [46], wherein, the cancer a K-Ras G12R associated cancer.

[52]. The method for treating cancer of [38], [45] or [46], the use in a method for the treatment of cancer of [42], [45] or [46], the use for the treatment of cancer of [43], [45] or [46], or the use for the manufacture of a medicament for the treatment of cancer of [44], [45] or [46], wherein, the cancer a K-Ras G12S associated cancer.

[53]. The method for treating cancer of [38], [45] or [46], the use in a method for the treatment of cancer of [42], [45] or [46], the use for the treatment of cancer of [43], [45] or [46], or the use for the manufacture of a medicament for the treatment of cancer of [44], [45] or [46], wherein, the cancer a K-Ras G12A associated cancer.

[54]. The method for treating cancer of [38], [45] or [46], the use in a method for the treatment of cancer of [42], [45] or [46], the use for the treatment of cancer of [43], [45] or [46], or the use for the manufacture of a medicament for the treatment of cancer of [44], [45] or [46], wherein, the cancer a K-Ras Q61H associated cancer.

[55]. The method for treating cancer of [38], [45] or [46], the use in a method for the treatment of cancer of [42], [45] or [46], the use for the treatment of cancer of [43], [45] or [46], or the use for the manufacture of a medicament for the treatment of cancer of [44], [45] or [46], wherein, the cancer a K-Ras wild type amplification associated cancer.

[56]. A process of preparing the compound of Formula (IB) of any one of [1] to [36], comprising the steps in the Scheme 1:

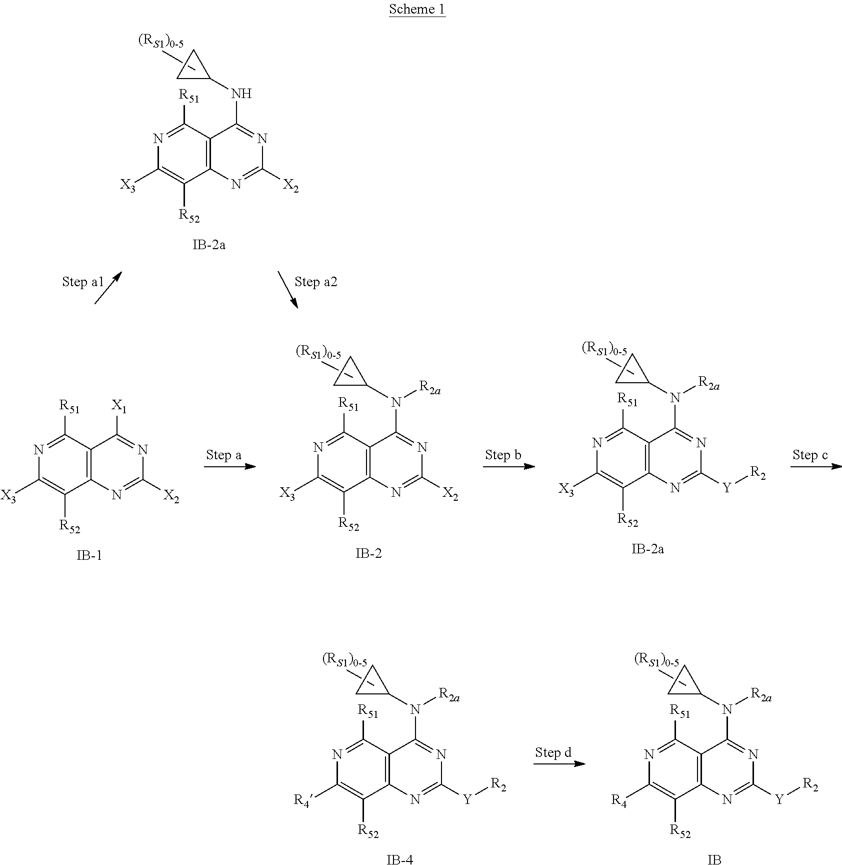

X₁, X₂ or X₃ at each occurrence is independently selected from a leaving group (such as —F, —Cl, —Br, —I, —OS(O)₂CF₃ or -OTs); preferably, X₁, X₂ or X₃ is selected from —Cl;
The definition of R₂ₐ, R₂, R₄, R₅₁, R₅₂, R_{S1} or Y is same as any one of [1] to [36];
R₄′ is R₄ with one or more protection groups.

[57]. An intermediate for preparing the compound of Formula (IB), comprising any one of the following formulas:

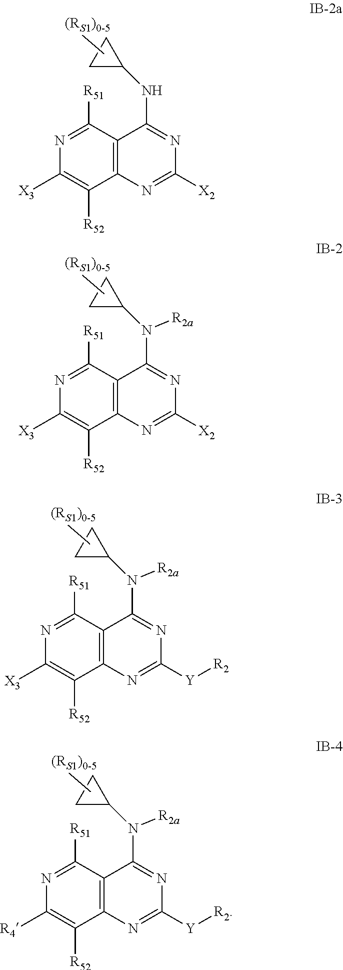

[58]. The intermediate of [57], wherein the intermediate is selected from any compound in the Table 14:

TABLE 14

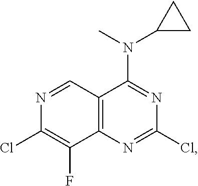 [SMILES: CN(c1nc(Cl)nc2c(F)c(Cl)ncc12)C1CC1]

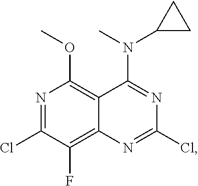 [SMILES: COc1nc(Cl)c(F)c2nc(Cl)nc(N(C)C3CC3)c12]

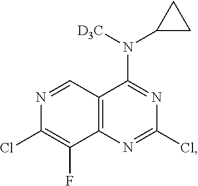 [SMILES: [2H]C([2H])([2H])N(c1nc(Cl)nc2c(F)c(Cl)ncc12)C1CC1]

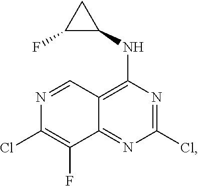 [SMILES: Fc1c(Cl)ncc2c(N[C@@H]3C[C@H]3F)nc(Cl)nc12]

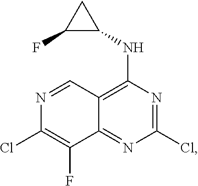 [SMILES: Fc1c(Cl)ncc2c(N[C@H]3C[C@@H]3F)nc(Cl)nc12]

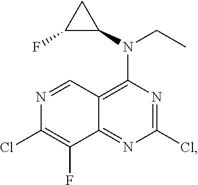 [SMILES: CCN(c1nc(Cl)nc2c(F)c(Cl)ncc12)[C@@H]1C[C@H]1F]

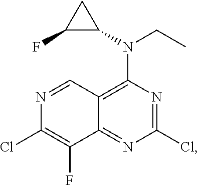 [SMILES: CCN(c1nc(Cl)nc2c(F)c(Cl)ncc12)[C@H]1C[C@@H]1F]

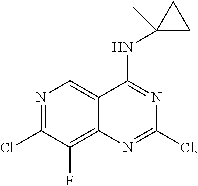 [SMILES: CC1(Nc2nc(Cl)nc3c(F)c(Cl)ncc23)CC1]

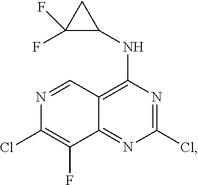 [SMILES: Fc1c(Cl)ncc2c(NC3CC3(F)F)nc(Cl)nc12]

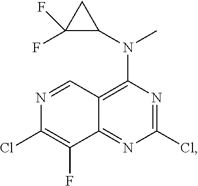 [SMILES: CN(c1nc(Cl)nc2c(F)c(Cl)ncc12)C1CC1(F)F]

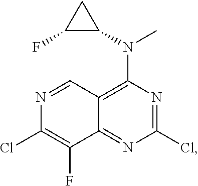 [SMILES: CN(c1nc(Cl)nc2c(F)c(Cl)ncc12)[C@H]1C[C@H]1F]

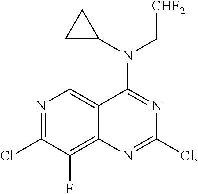 [SMILES: Fc1c(Cl)ncc2c(N(CC(F)F)C3CC3)nc(Cl)nc12]

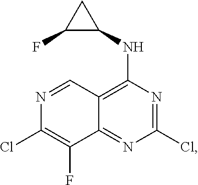 [SMILES: Fc1c(Cl)ncc2c(N[C@@H]3C[C@@H]3F)nc(Cl)nc12]

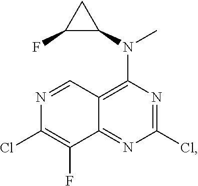 [SMILES: CN(c1nc(Cl)nc2c(F)c(Cl)ncc12)[C@@H]1C[C@@H]1F]

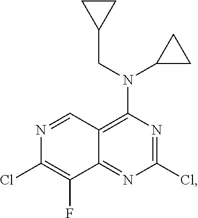 [SMILES: Fc1c(Cl)ncc2c(N(CC3CC3)C3CC3)nc(Cl)nc12]

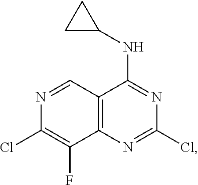 [SMILES: Fc1c(Cl)ncc2c(NC3CC3)nc(Cl)nc12]

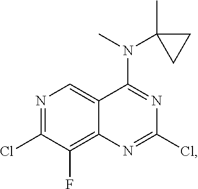 [SMILES: CN(c1nc(Cl)nc2c(F)c(Cl)ncc12)C1(C)CC1]

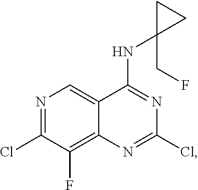 [SMILES: FCC1(Nc2nc(Cl)nc3c(F)c(Cl)ncc23)CC1]

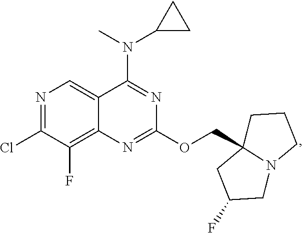 [SMILES: CN(c1nc(OC[C@@]23CCCN2C[C@H](F)C3)nc2c(F)c(Cl)ncc12)C1CC1]

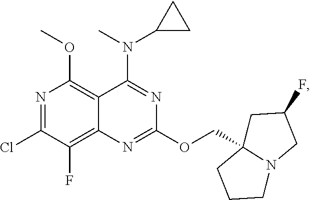 [SMILES: COc1nc(Cl)c(F)c2nc(OC[C@@]34CCCN3C[C@H](F)C4)nc(N(C)C3CC3)c12]

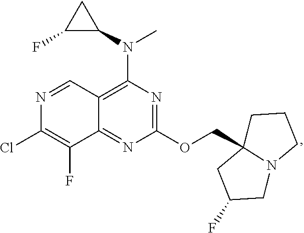 [SMILES: CN(c1nc(OC[C@@]23CCCN2C[C@H](F)C3)nc2c(F)c(Cl)ncc12)[C@@H]1C[C@H]1F]

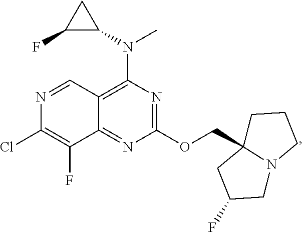 [SMILES: CN(c1nc(OC[C@@]23CCCN2C[C@H](F)C3)nc2c(F)c(Cl)ncc12)[C@H]1C[C@@H]1F]

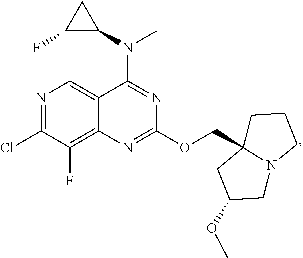 [SMILES: CO[C@H]1CN2CCC[C@@]2(COc2nc(N(C)[C@@H]3C[C@H]3F)c3cnc(Cl)c(F)c3n2)C1]

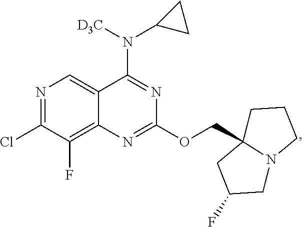 [SMILES: [2H]C([2H])([2H])N(c1nc(OC[C@@]23CCCN2C[C@H](F)C3)nc2c(F)c(Cl)ncc12)C1CC1]

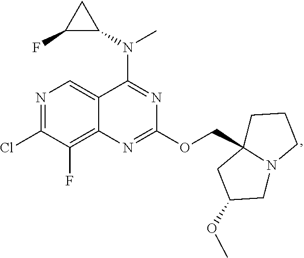 [SMILES: CO[C@H]1CN2CCC[C@@]2(COc2nc(N(C)[C@H]3C[C@@H]3F)c3cnc(Cl)c(F)c3n2)C1]

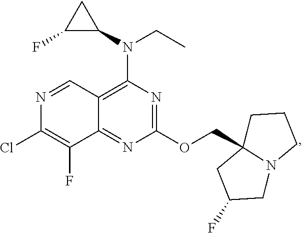 [SMILES: CCN(c1nc(OC[C@@]23CCCN2C[C@H](F)C3)nc2c(F)c(Cl)ncc12)[C@@H]1C[C@H]1F]

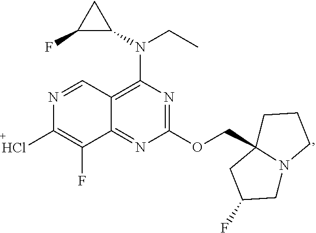 [SMILES: CCN(c1nc(OC[C@@]23CCCN2C[C@H](F)C3)nc2c(F)c(Cl)ncc12)[C@H]1C[C@@H]1F]

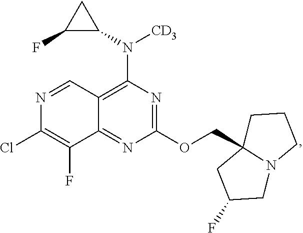 [SMILES: [2H]C([2H])([2H])N(c1nc(OC[C@@]23CCCN2C[C@H](F)C3)nc2c(F)c(Cl)ncc12)[C@H]1C[C@@H]1F]

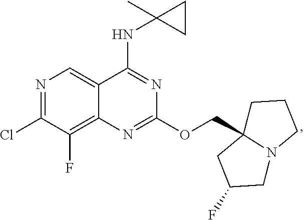 [SMILES: CC1(Nc2nc(OC[C@@]34CCCN3C[C@H](F)C4)nc3c(F)c(Cl)ncc23)CC1]

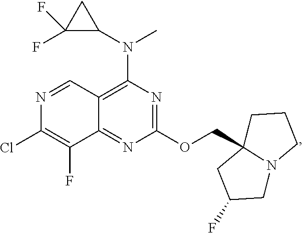 [SMILES: CN(c1nc(OC[C@@]23CCCN2C[C@H](F)C3)nc2c(F)c(Cl)ncc12)C1CC1(F)F]

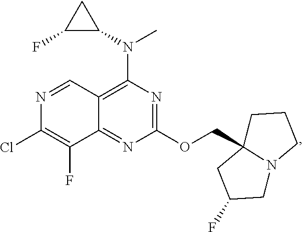 [SMILES: CN(c1nc(OC[C@@]23CCCN2C[C@H](F)C3)nc2c(F)c(Cl)ncc12)[C@H]1C[C@H]1F]

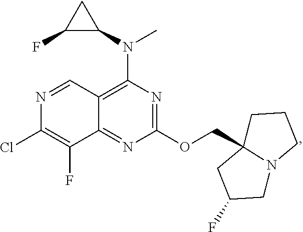 [SMILES: CN(c1nc(OC[C@@]23CCCN2C[C@H](F)C3)nc2c(F)c(Cl)ncc12)[C@@H]1C[C@@H]1F]

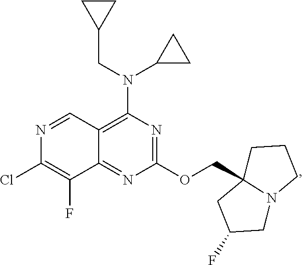 [SMILES: Fc1c(Cl)ncc2c(N(CC3CC3)C3CC3)nc(OC[C@@]34CCCN3C[C@H](F)C4)nc12]

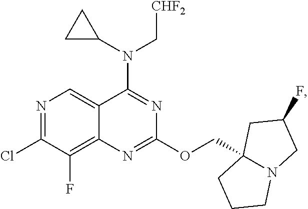 [SMILES: Fc1c(Cl)ncc2c(N(CC(F)F)C3CC3)nc(OC[C@@]34CCCN3C[C@H](F)C4)nc12]

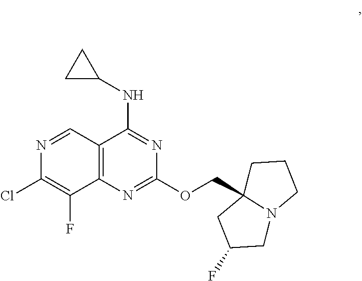 [SMILES: Fc1c(Cl)ncc2c(NC3CC3)nc(OC[C@@]34CCCN3C[C@H](F)C4)nc12]

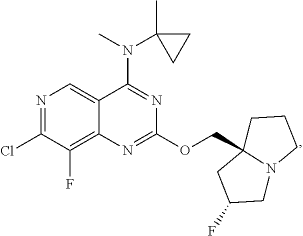 [SMILES: CN(c1nc(OC[C@@]23CCCN2C[C@H](F)C3)nc2c(F)c(Cl)ncc12)C1(C)CC1]

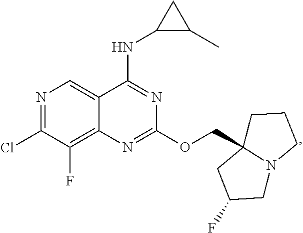 [SMILES: CC1CC1Nc1nc(OC[C@@]23CCCN2C[C@H](F)C3)nc2c(F)c(Cl)ncc12]

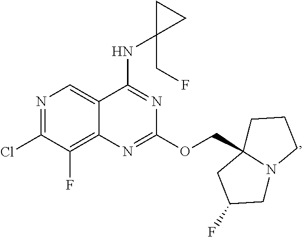 [SMILES: FCC1(Nc2nc(OC[C@@]34CCCN3C[C@H](F)C4)nc3c(F)c(Cl)ncc23)CC1]

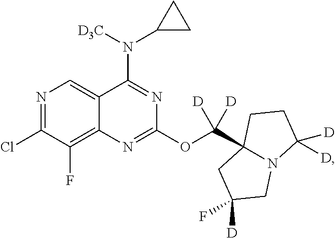

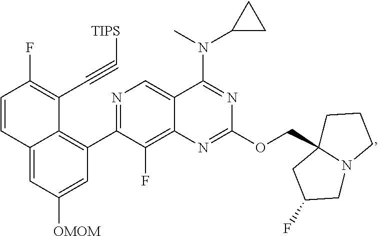

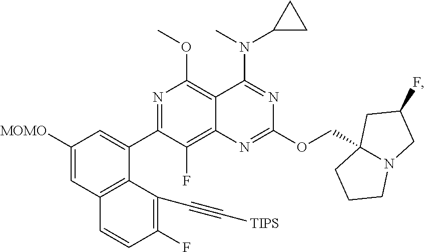

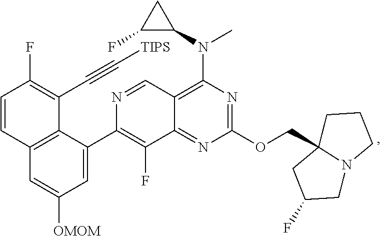

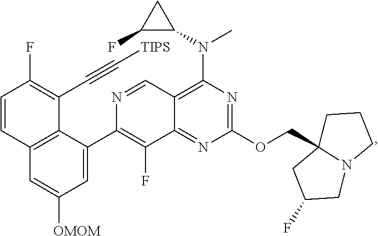

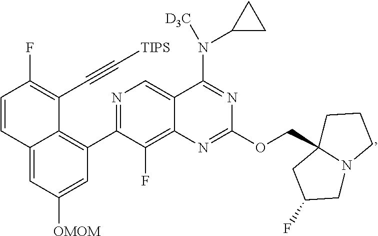

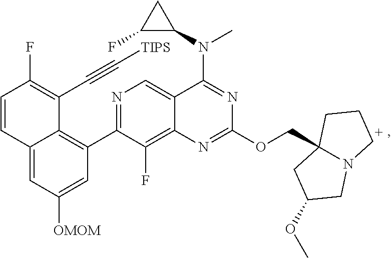

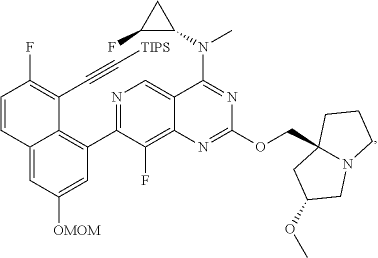

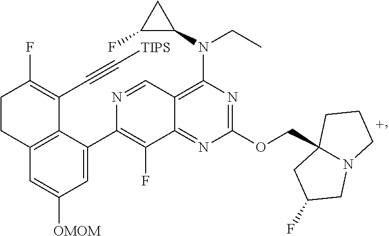

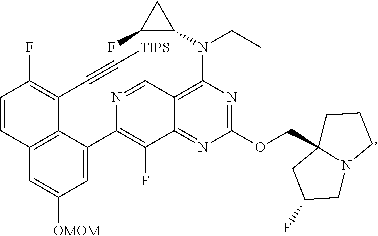

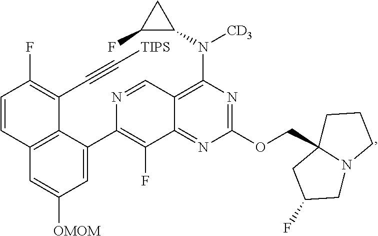

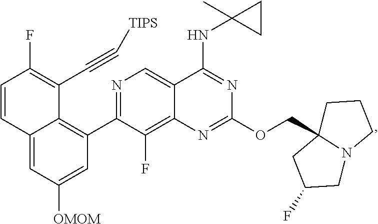

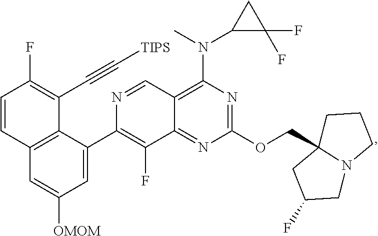

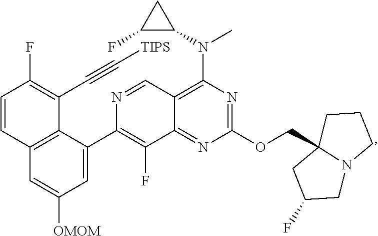

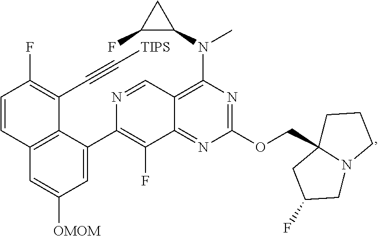

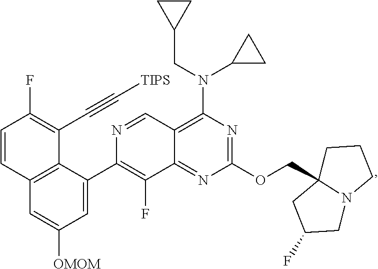

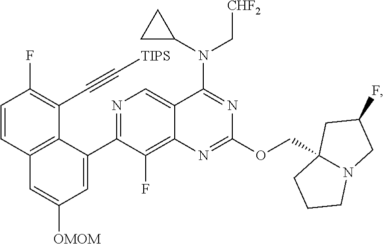

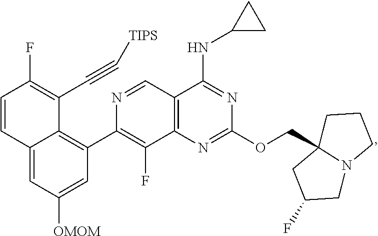

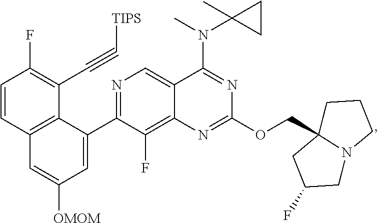

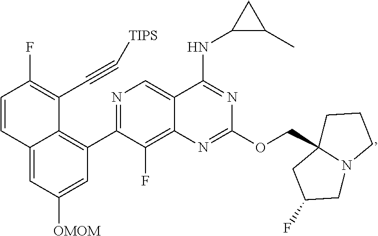

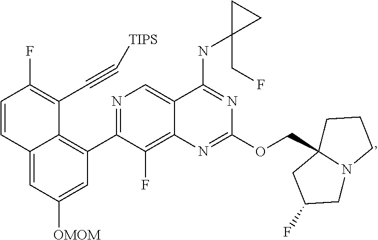

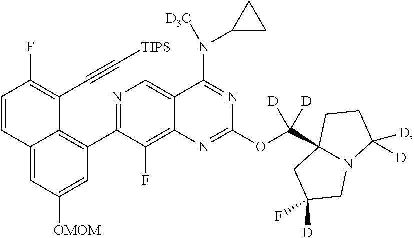

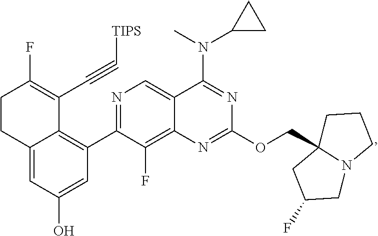

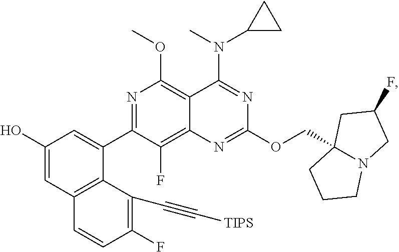

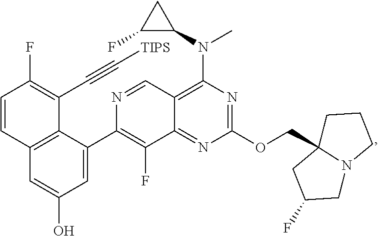

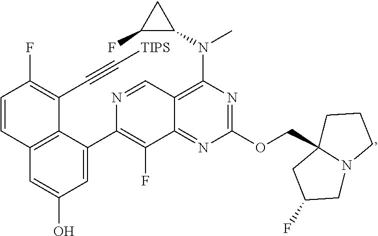

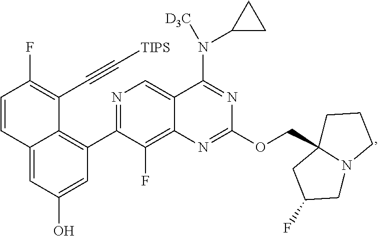

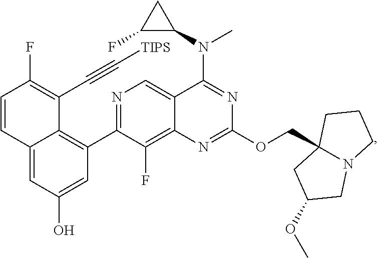

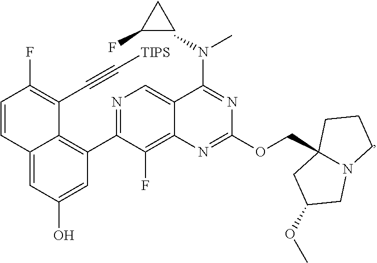

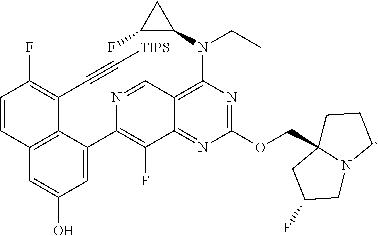

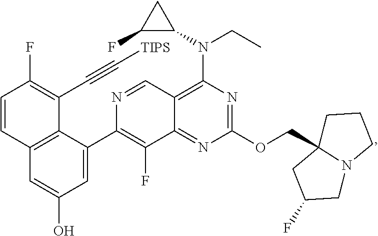

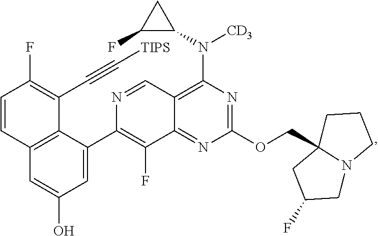

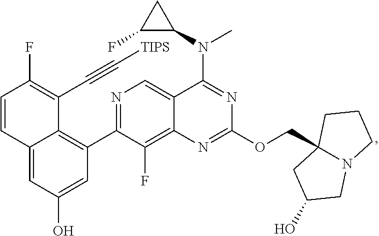

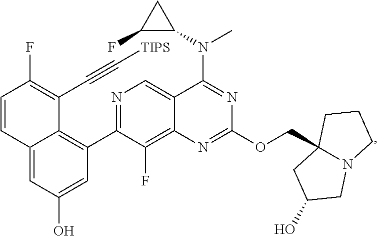

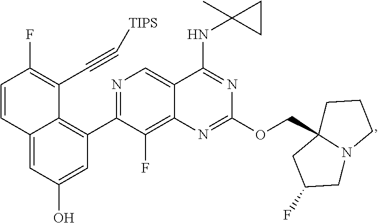

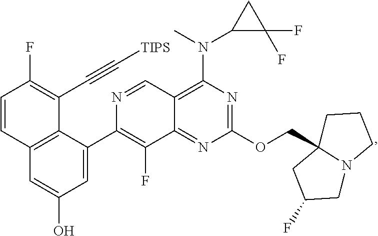

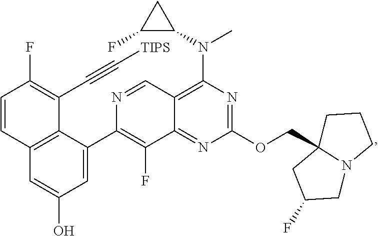

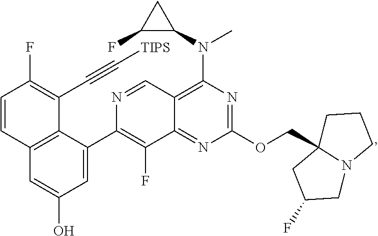

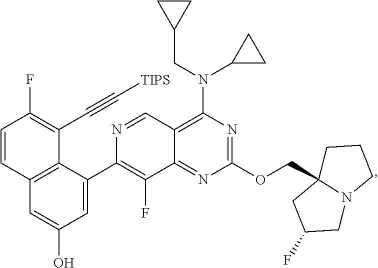

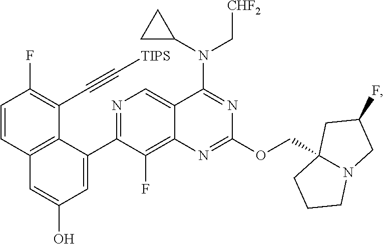

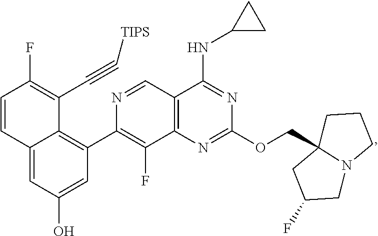

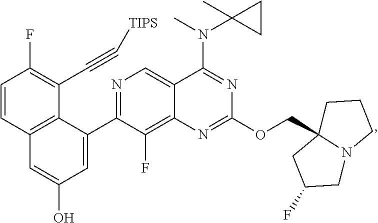

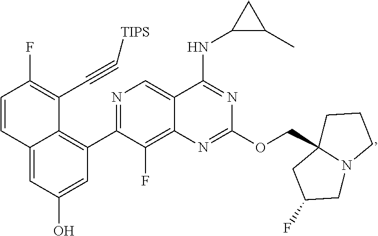

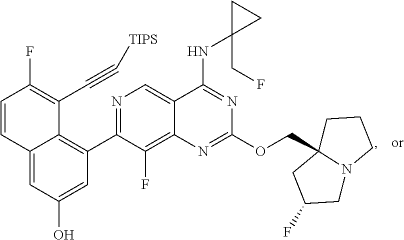

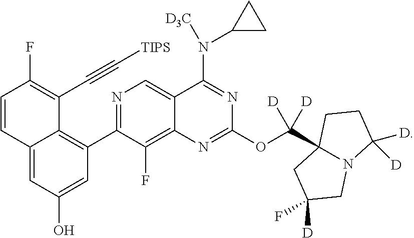

### Definitions

Unless defined otherwise, all technical and scientific terms used herein have the same meaning as is commonly understood by one of skill in the art to which this invention belongs. All patents, patent applications, and publications referred to herein are incorporated by reference.

The term “a”, “an”, “the” and similar terms, as used herein, unless otherwise indicated, are to be construed to cover both the singular and plural.

The term “halogen” or “halo”, as used interchangeably herein, unless otherwise indicated, refers to fluoro, chloro, bromo or iodo. The preferred halogen groups include —F, —Cl and —Br.

The term “alkyl”, as used herein, unless otherwise indicated, refers to saturated monovalent hydrocarbon radicals having straight or branched arrangement. C₁₋₁₀ in —C₁₋₁₀alkyl is defined to identify the group having 1, 2, 3, 4, 5, 6, 7, 8, 9 or 10 carbon atoms in a linear or branched arrangement. Non-limiting alkyl includes methyl, ethyl, propyl, isopropyl, n-butyl, isobutyl, sec-butyl, t-butyl, n-pentyl, 3-(2-methyl)butyl, 2-pentyl, 2-methylbutyl, neopentyl, n-hexyl, 2-hexyl and 2-methylpentyl.

The term “haloalkyl”, as used herein, unless otherwise indicated, refers to the above-mentioned alkyl substituted with one or more (for example 1, 2, 3, 4, 5, or 6) halogen (such as —F, —Cl or —Br). In some embodiments, the haloalkyl is interchangeable —C₁₋₁₀haloalkyl or haloC₁₋₁₀alkyl, wherein, C₁₋₁₀ in the —C₁₋₁₀haloaklyl or haloC₁₋₁₀alkyl indicates that the total carbon atoms of the alkyl are 1 to 10. In some embodiments, the —C₁₋₁₀haloalkyl is the —C₁₋₆haloalkyl. In some embodiments, the —C₁₋₆haloalkyl is the —C₁₋₃haloalkyl. In some embodiments, the —C₁₋₃haloalkyl is (methyl, ethyl, propyl or isopropyl) substituted with 1, 2, 3, 4, 5, or 6 —F; preferably, the —C₁₋₃haloalkyl is —CF₃.

The term “alkylene”, as used herein, unless otherwise indicated, refers to a divalent group obtained by removal of an additional hydrogen atom from an alkyl group defined above. In some embodiments, the alkylene is C₀₋₆alkylene. In some embodiments, the C₀₋₆alkylene is C₀₋₃alkylene. The C₀₋₆ in the front of the alkylene indicates the total carbon atoms in the alkylene are 0 to 6 and C₀ indicates the two ends of the alkylene are connected directly. Non-limiting alkylene includes methylene (i.e., —CH₂—), ethylene (i.e., —CH₂—CH₂— or —CH(CH₃)—) and propylene (i.e., —CH₂—CH₂—CH₂—, —CH(—CH₂—CH₃)— or —CH₂—CH(CH₃)—).

The term “alkenyl”, as used herein, unless otherwise indicated, refers to a straight or branch-chained hydrocarbon radical containing one or more double bonds and typically from 2 to 20 carbon atoms in length.

In some embodiments, the alkenyl is —C₂₋₁₀alkenyl. In some embodiments, the —C₂₋₁₀alkenyl is —C₂₋₆alkenyl which contains from 2 to 6 carbon atoms. Non-limiting alkenyl includes ethenyl, propenyl, butenyl, 2-methyl-2-buten-1-yl, hepetenyl, octenyl and the like.

The term “haloalkenyl”, as used herein, unless otherwise indicated, refers to the above-mentioned alkenyl substituted with one or more (for example 1, 2, 3, 4, 5, or 6) halogen (such as —F, —Cl or —Br). In some embodiments, the haloalkenyl is interchangeable —C₂₋₁₀haloalkenyl or haloC₂₋₁₀alkenyl, wherein, C₂₋₁₀ in the —C₂₋₁₀haloaklenyl or haloC₂₋₁₀alkenyl indicates that the total carbon atoms of the alkenyl are 2 to 10. In some embodiments, the —C₂₋₁₀haloalkenyl is the —C₂₋₆haloalkenyl. In some embodiments, the —C₂₋₆haloalkenyl is the —C₂₋₃haloalkenyl. In some embodiments, the —C₂₋₃haloalkenyl is (ethenyl or propenyl) substituted with 1, 2, 3, 4, 5, or 6 —F.

The term “alkynyl”, as used herein, unless otherwise indicated, refers to a straight or branch-chained hydrocarbon radical containing one or more triple bonds and typically from 2 to 20 carbon atoms in length. In some embodiments, the alkynyl is —C₂₋₁₀alkynyl. In some embodiments, the —C₂₋₁₀alkynyl is —C₂₋₆alkynyl which contains from 2 to 6 carbon atoms. Non-limiting alkynyl includes ethynyl, 1-propynyl, 1-butynyl, heptynyl, octynyl and the like.

The term “haloalkynyl”, as used herein, unless otherwise indicated, refers to the above-mentioned alkynyl substituted with one or more (for example 1, 2, 3, 4, 5, or 6) halogen (such as —F, —Cl or —Br). In some embodiments, the haloalkynyl is interchangeable —C₂₋₁₀haloalkynyl or haloC₂₋₁₀alkynyl, wherein, C₂₋₁₀ in the —C₂₋₁₀haloaklynyl or haloC₂₋₁₀alkynyl indicates that the total carbon atoms of the alkynyl are 2 to 10. In some embodiments, the —C₂₋₁₀haloalkynyl is the —C₂₋₆haloalkynyl. In some embodiments, the —C₂₋₆haloalkynyl is the —C₂₋₃haloalkynyl. In some embodiments, the —C₂₋₃haloalkynyl is (ethynyl or propynyl) substituted with 1, 2, 3, 4, 5, or 6 —F.

The term “alkoxy”, as used herein, unless otherwise indicated, refers to oxygen ethers formed from the previously described alkyl groups.

The term “haloalkoxy”, as used herein, unless otherwise indicated, refers to the above-mentioned alkoxy substituted with one or more (for 1, 2, 3, 4, 5, or 6) halogen (—F, —Cl or —Br). In some embodiment, the haloalkoxy is interchangeable —C₁₋₁₀haloalkoxy or haloC₁₋₁₀alkoxy. In some embodiments, the haloalkoxy is interchangeable —C₁₋₆haloalkoxy or haloC₁₋₆alkoxy, wherein, C₁₋₆ in the —C₁₋₆haloakloxy or haloC₁₋₆alkoxy indicates that the total carbon atoms of the alkoxy are 1 to 6. In some embodiments, the —C₁₋₆haloalkoxy is the —C₁₋₃haloalkoxy. In some embodiments, the —C₁₋₃haloalkoxy is (methoxy, ethoxy, propoxy or isopropoxy) substituted with 1, 2, 3, 4, 5, or 6 —F; preferably, the —C₁₋₃haloalkoxy is —OCF₃.

The term “carbocyclic ring”, as used herein, unless otherwise indicated, refers to a totally saturated or partially saturated monocyclic, bicyclic, bridged, fused, or spiro non-aromatic ring only containing carbon atoms as ring members. The term “carbocyclyl” as used herein, unless otherwise indicated, means a monovalent group obtained by removal of a hydrogen atom on the ring carbon atom from the carbocyclic ring defined in the present invention. The carbocyclic ring is interchangeable with the carbocyclyl ring in the present invention. In some embodiments, the carbocyclic ring is a three to twenty membered (such as 3-, 4-, 5-, 6-, 7-, 8-, 9-, 10-, 11-, 12-, 13-, 14-, 15-, 16-, 17-, 18-, 19- or 20-membered) carbocyclic ring and is either fully saturated or has one or more degrees of unsaturation. Multiple degrees of substitution, for example, one, two, three, four, five or six, are included within the present definition. The carbocyclic ring includes a cycloalkyl ring in which all ring carbon atoms are saturated, a cycloalkenyl ring which contains at least one double bond (preferred contain one double bond), and a cycloalkynyl ring which contains at least one triple bond (preferred contain one triple bond). Cycloalkyl includes but not limited to cyclopropyl, cyclobutyl, cyclopentyl, cyclohexyl, cycloheptyl, cyclooctyl, cyclononyl, cyclodecyl and the like. Cycloalkenyl includes but not limited to cyclopentenyl, cyclohexenyl, cycloheptenyl, cyclooctenyl, cyclononenyl, cyclodecenyl and the like. The carbocyclyl ring includes a monocyclic carbocyclyl ring, and a bicyclic or polycyclic carbocyclyl ring in which one, two or three or more atoms are shared between the rings. The term “spirocyclic carbocyclic ring” refers to a carbocyclic ring in which each of the rings only shares one ring atom with the other ring. In some embodiments, the spirocyclic ring is bicyclic spirocyclic ring. The spirocyclic carbocyclic ring includes a spirocyclic cycloalkyl ring and a spirocyclic cycloalkenyl ring and a spirocyclic cycloalkynyl ring. The term “fused carbocyclic ring” refers to a carbocyclic ring in which each of the rings shares two adjacent ring atoms with the other ring. In some embodiments, the fused ring is a bicyclic fused ring. The fused carbocyclic ring includes a fused cycloalkyl ring and a fused cycloalkenyl ring and a fused cycloalkynyl ring. A monocyclic carbocyclic ring fused with an aromatic ring (such as phenyl) is included in the definition of the fused carbocyclic ring. The term “bridged carbocyclic ring” refers to a carbocyclic ring that includes at least two bridgehead carbon ring atoms and at least one bridging carbon atom. In some embodiments, the bridged ring is bicyclic bridged ring. The bridged carbocyclic ring includes a bicyclic bridged carbocyclic ring which includes two bridgehead carbon atoms and a polycyclic bridged carbocyclic ring which includes more than two bridgehead carbon atoms. The bridged carbocyclic ring includes a bridged cycloalkyl ring, a bridged cycloalkenyl ring and a bridged cycloalkynyl ring. Examples of monocyclic carbocyclyl and bicyclic carbocyclyl include but not limit to cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, and 1-cyclohex-3-enyl.

The term “heterocyclic ring”, as used herein, unless otherwise indicated, refers to a totally saturated or partially saturated monocyclic, bicyclic, bridged, fused, or spiro non-aromatic ring containing not only carbon atoms as ring members and but also containing one or more (such as 1, 2, 3, 4, 5, or 6) heteroatoms as ring members. Preferred heteroatoms include N, O, S, N-oxides, sulfur oxides, and sulfur dioxides. The term “heterocyclyl” as used herein, unless otherwise indicated, means a monovalent group obtained by removal of a 50 hydrogen atom on the ring carbon atom or the ring heteroatom from the heterocyclic ring defined in the present invention. The heterocyclic ring is interchangeable with the heterocyclyl ring in the present invention. In some embodiments, the heterocyclic ring is a three to twenty membered (such as 3-, 4-, 5-, 6-, 7-, 8-, 9-, 10-, 11-, 12-, 13-, 14-, 15-, 16-, 17-, 18-, 19- or 20-membered) heterocyclic ring and is either fully saturated or has one or more degrees of unsaturation. Multiple degrees of substitution, for example, one, two, three, four, five or six, 55 are included within the present definition. The heterocyclic ring includes a heterocycloalkyl ring in which all ring carbon atoms are saturated, a heterocycloalkenyl ring which contains at least one double bond (preferred contain one double bond), and a heterocycloalkynyl ring which contains at least one triple bond (preferred contain one triple bond). The heterocyclyl ring includes a monocyclic heterocyclyl ring, and a bicyclic or polycyclic heterocyclyl ring in which one, two or three or more atoms are shared between the rings. The term “spirocyclic heterocyclic ring” refers to a heterocyclic ring in which each of the rings only shares one ring atom with the other ring. In some embodiments, the spirocyclic ring is bicyclic spirocyclic ring. The spirocyclic heterocyclic ring includes a spirocyclic heterocycloalkyl ring and a spirocyclic heterocycloalkenyl ring and a spirocyclic heterocycloalkynyl ring. The term “fused heterocyclic ring” refers to a heterocyclic ring in which each of the rings shares two adjacent ring atoms with the other ring. In some embodiments, the fused ring is a bicyclic fused ring. The fused heterocyclic ring includes a fused heterocycloalkyl ring and a fused heterocycloalkenyl ring and a fused heterocycloalkynyl ring. A monocyclic heterocyclic ring fused with an aromatic ring (such as phenyl) is included in the definition of the fused heterocyclic ring. The term “bridged heterocyclic ring” refers to a heterocyclic ring that includes at least two bridgehead ring atoms and at least one bridging atom. In some embodiments, the bridged ring is bicyclic bridged ring. The bridged heterocyclic ring includes a bicyclic bridged heterocyclic ring which includes two bridgehead atoms and a polycyclic bridged heterocyclic ring which includes more than two bridgehead atoms. The bridged heterocyclic ring includes a bridged heterocycloalkyl ring, a bridged heterocycloalkenyl ring and a bridged heterocycloalkynyl ring. Examples of such heterocyclyl include but are not limited to azetidinyl, pyrrolidinyl, piperidinyl, piperazinyl, oxopiperazinyl, oxopiperidinyl, oxoazepinyl, azepinyl, tetrahydrofuranyl, dioxolanyl, tetrahydroimidazolyl, tetrahydrothiazolyl, tetrahydrooxazolyl, tetrahydropyranyl, morpholinyl, thiomorpholinyl, thiamorpholinyl sulfoxide, thiamorpholinyl sulfone and oxadiazolyl.

The term “aryl”, as used herein, unless otherwise indicated, refers to a mono or polycyclic aromatic ring system only containing carbon ring atoms. The preferred aryls are monocyclic or bicyclic 6-10 membered aromatic rings. Phenyl and naphthyl are preferred aryls.

The term “heteroaryl”, as used herein, unless otherwise indicated, refers to an aromatic ring containing carbons and one or more (such as 1, 2, 3 or 4) heteroatoms selected from N, O or S. The heteroaryl may be monocyclic or polycyclic. A monocyclic heteroaryl group may have 1 to 4 heteroatoms in the ring, while a polycyclic heteroaryl may contain 1 to 10 heteroatoms. A polycyclic heteroaryl ring may contain fused ring junction, for example, bicyclic heteroaryl is a polycyclic heteroaryl. Bicyclic heteroaryl rings may contain from 8 to 12 member atoms. Monocyclic heteroaryl rings may contain from 5 to 8 member atoms (carbons and heteroatoms), preferred monocyclic heteroaryl is 5 membered heteroaryl including 1, 2, 3 or 4 heteroatoms selected from N, O or S, or 6 membered heteroaryl including 1 or 2 heteroatoms selected from N. Examples of heteroaryl groups include, but not limited to thienyl, furanyl, imidazolyl, isoxazolyl, oxazolyl, pyrazolyl, pyrrolyl, thiazolyl, thiadiazolyl, triazolyl, pyridyl, pyridazinyl, indolyl, azaindolyl, indazolyl, benzimidazolyl, benzofuranyl, benzothienyl, benzisoxazolyl, benzoxazolyl, benzopyrazolyl, benzothiazolyl, benzothiadiazolyl, benzotriazolyladeninyl, quinolinyl or isoquinolinyl.

The term “one or more”, as used herein, unless otherwise indicated, refers to one or more than one. In some embodiments, “one or more” refers to 1, 2, 3, 4, 5 or 6. In some embodiments, “one or more” refers to 1, 2, 3 or 4. In some embodiments, “one or more” refers to 1, 2, or 3. In some embodiments, “one or more” refers to 1 or 2. In some embodiments, “one or more” refers to 1. In some embodiments, “one or more” refers to 2. In some embodiments, “one or more” refers to 3. In some embodiments, “one or more” refers to 4. In some embodiments, “one or more” refers to 5. In some embodiments, “one or more” refers to 6.

The term “substituted”, as used herein, unless otherwise indicated, refers to a hydrogen atom on the carbon atom or a hydrogen atom on the nitrogen atom is replaced by a substituent. When one or more substituents are substituted on a ring in the present invention, it means that each of substituents may be respectively independently substituted on every ring atom of the ring including but not limited to a ring carbon atom or a ring nitrogen atom. In addition, when the ring is a polycyclic ring, such as a fused ring, a bridged ring or a spiro ring, each of substituents may be respectively independently substituted on every ring atom of the polycyclic ring.

The term “oxo” refers to oxygen atom together with the attached carbon atom forms the group.

The term “composition”, as used herein, is intended to encompass a product comprising the specified ingredients in the specified amounts, as well as any product which results, directly or indirectly, from combinations of the specified ingredients in the specified amounts. Accordingly, pharmaceutical compositions containing the compounds of the present invention as the active ingredient as well as methods of preparing the instant compounds are also part of the present invention. Furthermore, some of the crystalline forms for the compounds may exist as polymorphs and as such are intended to be included in the present invention. In addition, some of the compounds may form solvates with water (i.e., hydrates) or common organic solvents and such solvates are also intended to be encompassed within the scope of this invention.

The term “pharmaceutically acceptable salt” refers to a salt prepared from pharmaceutically acceptable non-toxic bases or acids. When the compound of the present invention is acidic, its corresponding salt can be conveniently prepared from pharmaceutically acceptable non-toxic bases, including inorganic bases and organic bases. When the compound of the present invention is basic, its corresponding salt can be conveniently prepared from pharmaceutically acceptable non-toxic acids, including inorganic and organic acids. Since the compounds in the present invention are intended for pharmaceutical use they are preferably provided in substantially pure form, for example at least 60% pure, more suitably at least 75% pure, especially at least 98% pure (% are on a weight for weight basis).

The present invention includes within its scope the prodrug of the compounds of this invention. In general, such prodrug will be functional derivatives of the compounds that are readily converted in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the subject. Conventional procedures for the selection and preparation of suitable prodrug derivatives are described, for example, in “Design of Prodrugs”, ed. H. Bundgaard, Elsevier, 1985.

It is intended that the definition of any substituent or variable at a particular location in a molecule be independent of its definitions elsewhere in that molecule. It is understood that substituents and substitution patterns on the compounds of this invention can be selected by one of ordinary skill in the art to provide compounds that are chemically stable and that can be readily synthesized by techniques know in the art as well as those methods set forth herein.

The present invention includes all stereoisomers of the compound and pharmaceutically acceptable salts thereof. Further, mixtures of stereoisomers as well as isolated specific stereoisomers are also included. During the course of the synthetic procedures used to prepare such compounds or in using racemization or epimerization procedures known to those skilled in the art, the products of such procedures can be a mixture of stereoisomers. The term “stereoisomer” as used in the present invention refers to an isomer in which atoms or groups of atoms in the molecule are connected to each other in the same order but differ in spatial arrangement, including conformational isomers and configuration isomers. The configuration isomers include geometric isomers and optical isomers, and optical isomers mainly include enantiomers and diastereomers. The invention includes all possible stereoisomers of the compound.

The present invention is intended to include all isotopes of atoms occurring in the present compounds. Isotopes include those atoms having the same atomic number but different mass numbers. By way of general example and without limitation, isotopes of hydrogen include deuterium and tritium. The isotopes of hydrogen can be denoted as ¹H (hydrogen), ²H (deuterium) and ³H (tritium). They are also commonly denoted as D for deuterium and T for tritium. In the application, CD₃ denotes a methyl group wherein all of the hydrogen atoms are deuterium. Isotopes of carbon include ¹³C and ¹⁴C. Isotopically-labeled compounds of the invention can generally be prepared by conventional techniques known to those skilled in the art or by processes analogous to those described herein, using an appropriate isotopically-labeled reagent in place of the non-labeled reagent.

The term “deuterated derivative”, used herein, unless otherwise indicated, refers to a compound having the same chemical structure as a reference compound, but with one or more hydrogen atoms replaced by a deuterium atom (“D”). It will be recognized that some variation of natural isotopic abundance occurs in a synthesized compound depending on the origin of chemical materials used in the synthesis. The concentration of naturally abundant stable hydrogen isotopes, notwithstanding this variation is small and immaterial as compared to the degree of stable isotopic substitution of deuterated derivative described herein. Thus, unless otherwise stated, when a reference is made to a “deuterated derivative” of a compound of the disclosure, at least one hydrogen is replaced with deuterium at well above its natural isotopic abundance (which is typically about 0.015%) In some embodiments, the deuterated derivative of the disclosure have an isotopic enrichment factor for each deuterium atom, of at least 3500 (52.5% deuterium incorporation at each designated deuterium) at least 4500, (67.5% deuterium incorporation), at least 5000 (75% deuterium incorporation) at least 5500 (82.5% deuterium incorporation), at least 6000 (90% deuterium incorporation), at lease 6333.3 (95% deuterium incorporation, at least 6466.7 (97% deuterium incorporation, or at least 6600 (99% deuterium incorporation).

When a tautomer of the compound in the present invention exists, the present invention includes any possible tautomer and pharmaceutically acceptable salts thereof, and mixtures thereof, except where specifically stated otherwise.

The “conjugated form” refers to herein that the compound described herein is conjugated to another agent through a linker or not through a linker, wherein, the compound functions as a binder or a inhibitor of K-Ras protein (including K-Ras G12C, K-Ras G12D, K-Ras G12V, K-Ras G13D, K-Ras G12R, K-Ras G12S, K-Ras G12A, K-Ras Q61H mutant protein and K-Ras wild type protein) For example, the conjugated form is a PROTAC molecule, e.g. the compound is incorporated into proteolysis targeting chimeras (PROTACs). A PROTAC is a bifunctional molecule, with one portion capable of engaging an E3 ubiquitin ligase, and the other portion having the ability to bind to a target protein meant for degradation by the cellular protein quality control machinery. Recruitment of the target protein to the specific E3 ligase results in its tagging for destruction (i.e., ubiquitination) and subsequent degradation by the proteasome. Any E3 ligase can be used. Preferably, the portion of the PROTAC that engages the E3 ligase is connected to the portion of the PROTAC that engages the target protein via a linker which consists of a variable chain of atoms. Recruitment of K-Ras protein to the E3 ligase will thus result in the destruction of the K-Ras protein. The variable chain of atoms can include, for example, rings, heteroatoms, and/or repeating polymeric units. It can be rigid or flexible. It can be attached to the two portions described above using standard techniques in the art of organic synthesis.

The pharmaceutical compositions of the present invention comprise a compound in present invention (or a pharmaceutically acceptable salt thereof) as an active ingredient, a pharmaceutically acceptable carrier and optionally other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, a compound of Formula (IB), a stereoisomer thereof, a pharmaceutically acceptable salt thereof, a pharmaceutically acceptable salt of the stereoisomer thereof, a prodrug thereof, a deuterated molecule thereof or a conjugated form thereof as defined herein can be combined as the active ingredient in intimate admixture with a pharmaceutical carrier according to conventional pharmaceutical compounding techniques. The carrier may take a wide variety of forms depending on the form of preparation desired for administration, e.g. oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as oil-in-water emulsion or as a water-in-oil liquid emulsion. In addition to the common dosage forms set out above, the compound in the present invention or a pharmaceutically acceptable salt thereof may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and a compound or a pharmaceutically acceptable salt. The compounds of the present invention or pharmaceutically acceptable salts thereof can also be included in pharmaceutical compositions in combination with one or more other therapeutically active compounds.

The pharmaceutical carrier employed can be, for example, a solid, liquid or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen. In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder or the like. Further, the compositions can be in a form suitable for use in transdermal devices. These formulations may be prepared, utilizing a compound in the present invention or a pharmaceutically acceptable salt thereof, via conventional processing methods. As an example, a cream or ointment is prepared by admixing hydrophilic material and water, together with about 0.05 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including antioxidants) and the like. Furthermore, other adjuvants can be included to render the formulation isotonic with the blood of the intended recipient. Compositions containing a compound described herein or pharmaceutically acceptable salts thereof, may also be prepared in powder or liquid concentrate form.

Unless otherwise apparent from the context, when a value is expressed as “about” X or “approximately” X, the stated value of X will be understood to be accurate to ±10%, preferably, ±5%, ±2%.

The term “subject” refers to an animal. In some embodiments, the animal is a mammal. A subject also refers to for example, primates (e.g., humans), cows, sheep, goats, horses, dogs, cats, rabbits, rats, mice, fish, birds and the like. In certain embodiments, the subject is a human. A “patient” as used herein refers to a human subject. As used herein, a subject is “in need of” a treatment if such subject would benefit biologically, medically or in quality of life from such treatment. In some embodiments, the subject has experienced and/or exhibited at least one symptom of cancer to be treated and/or prevented. In some embodiments, the subject has been identified or diagnosed as having a cancer having wild type K-Ras or a K-Ras G12A, K-Ras G12C, K-Ras G12D, K-Ras G12R, K-Ras G12S, K-Ras G12V, K-Ras G13D and/or K-Ras Q61H mutation

The term “inhibition”, “inhibiting” or “inhibit” refers to the reduction or suppression of a given condition, symptom, or disorder, or disease, or a significant decrease in the baseline activity of a biological activity or process.

The term “treat”, “treating” or “treatment” of any disease or disorder refers in one embodiment, to ameliorating the disease or disorder (i.e., slowing or arresting or reducing the development of the disease or at least one of the clinical symptoms thereof). In another embodiment, “treat”, “treating” or “treatment” refers to alleviating or ameliorating at least one physical parameter including those which may not be discernible by the patient. In yet another embodiment, “treat”, “treating” or “treatment” refers to modulating the disease or disorder, either physically, (e.g., stabilization of a discernible symptom), physiologically, (e.g., stabilization of a physical parameter), or both. In yet another embodiment, “treat”, “treating” or “treatment” refers to preventing or delaying the onset or development or progression of the disease or disorder.

As used herein, “K-Ras G12A” refers to a mutant form of a mammalian K-Ras protein that contains an amino acid substitution of an alanine for a glycine at amino acid position 12. A “K-Ras G12A inhibitor” refers to a compound is capable of negatively modulating or inhibiting all or a portion of the function of K-Ras G12A. A “K-Ras G12A associated cancer” as used herein refers to a cancer associated with or mediated by or having a K-Ras G12A mutation.

As used herein, “K-Ras G12C” refers to a mutant form of a mammalian K-Ras protein that contains an amino acid substitution of a cysteine for a glycine at amino acid position 12. A “K-Ras G12C inhibitor” refers to a compound is capable of negatively modulating or inhibiting all or a portion of the function of K-Ras G12C.

A “K-Ras G12C associated cancer” as used herein refers to a cancer associated with or mediated by or having a K-Ras G12C mutation.

As used herein, “K-Ras G12D” refers to a mutant form of a mammalian K-Ras protein that contains an amino acid substitution of an aspartic acid for a glycine at amino acid position 12. A “K-Ras G12D inhibitor” refers to a compound is capable of negatively modulating or inhibiting all or a portion of the function of K-Ras G12D. A “K-Ras G12D associated cancer” as used herein refers to a cancer associated with or mediated by or having a K-Ras G12D mutation.

As used herein, “K-Ras G12R” refers to a mutant form of a mammalian K-Ras protein that contains an amino acid substitution of an arginine for a glycine at amino acid position 12. A “K-Ras G12R inhibitor” refers to a compound is capable of negatively modulating or inhibiting all or a portion of the function of K-Ras G12R. A “K-Ras G12R associated cancer” as used herein refers to a cancer associated with or mediated by or having a K-Ras G12R mutation.

As used herein, “K-Ras G12S” refers to a mutant form of a mammalian K-Ras protein that contains an amino acid substitution of a serine for a glycine at amino acid position 12. A “K-Ras G12S inhibitor” refers to a compound is capable of negatively modulating or inhibiting all or a portion of the function of K-Ras G12S.

A “K-Ras G12S associated cancer” as used herein refers to a cancer associated with or mediated by or having a K-Ras G12S mutation.

As used herein, “K-Ras G12V” refers to a mutant form of a mammalian K-Ras protein that contains an amino acid substitution of a valine for a glycine at amino acid position 12. A “K-Ras G12V inhibitor” refers to a compound is capable of negatively modulating or inhibiting all or a portion of the function of K-Ras G12V.

A “K-Ras G12V associated cancer” as used herein refers to a cancer associated with or mediated by or having a K-Ras G12V mutation.

As used herein, “K-Ras G13D” refers to a mutant form of a mammalian K-Ras protein that contains an amino acid substitution of an aspartic acid for a glycine at amino acid position 13. A “K-Ras G13D inhibitor” refers to a compound is capable of negatively modulating or inhibiting all or a portion of the function of K-Ras G13D. A “K-Ras G13D associated cancer” as used herein refers to a cancer associated with or mediated by or having a K-Ras G13D mutation.

As used herein, “K-Ras Q61H” refers to a mutant form of a mammalian K-Ras protein that contains an amino acid substitution of a histidine for a glutamine at amino acid position 61. A “K-Ras Q61H inhibitor” refers to a compound is capable of negatively modulating or inhibiting all or a portion of the function of K-Ras Q61H. A “K-Ras Q61H associated cancer” as used herein refers to a cancer associated with or mediated by or having a K-Ras Q61H mutation.

All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed.

These and other aspects will become apparent from the following written description of the invention.

### METHODS OF PREPARATION

Compounds of the present invention can be synthesized from commercially available reagents using the synthetic methods and reaction schemes described herein. The examples which outline specific synthetic route, and the generic schemes below are meant to provide guidance to the ordinarily skilled synthetic chemist, who will readily appreciate that the solvent, concentration, reagent, protecting group, order of synthetic steps, time, temperature, and the like can be modified as necessary, well within the skill and judgment of the ordinarily skilled artisan.

### EXAMPLES

The following examples are provided to better illustrate the present invention. All parts and percentages are by weight and all temperatures are degrees Celsius, unless explicitly stated otherwise. The following abbreviations in the Table 15 have been used in the examples:

TABLE 15

| | |
|---|---|
| DMF | N,N-dimethylformamide |
| EA/EtOAc | Ethyl acetate |
| Hex | Hexane |
| MeOH | Methanol |
| DCM | Dichloromethane |
| DCE | 1,2-dichloroethane |
| EtOH | Ethanol |
| THF | Tetrahydrofuran |
| DIEA/DIPEA | N,N-Diisopropylethylamine |
| Pd(PPh₃)₄ | Tetrakis(triphenylphosphine)palladium |
| Pd(dppf)Cl₂ | [1,1′-Bis(diphenylphosphino)ferrocene]dichloro- |
| | palladium(II) |
| TFA | 2,2,2-trifluoroacetic acid |
| ACN/MeCN/CH₃CN | Acetonitrile |
| Et₃N/TEA | Triethylamine |
| NIS | N-Iodosuccinimide |
| DMSO | Dimethyl sulfoxide |
| NCS | N-chlorosuccinimide |
| TBSCl | Tert-butyldimethylsilyl chloride |
| TMSCl | Trimethylsilyl chloride |
| MOMCl | Methoxymethyl chloride |
| MsCl | Methanesulfonyl chloride |
| LAH | Lithium aluminum hydride |
| LDA | Lithium diisopropylamide |
| LiHMDS | Lithium hexamethyldisilazide |
| B₂(Pin)₂ | Bis(pinacolato)diboron |
| NFSI | N-Fluorobenzenesulfonimide |
| MTBE | Methyl tert-butyl ether |
| DMAP | N,N-dimethylpyridin-4-amine |
| DABCO | Triethylenediamine |
| TABF | Tetrabutylammonium fluoride |
| m-CPBA | 3-Chloroperbenzoic acid |
| NMP | N-methylpyrrolidone |
| rt/RT/R.T | Room temperature |
| min(s) | minute(s) |
| h/hr(s) | hour(s) |
| aq | aqueous |
| Sat. | saturated |
| TLC | Thin layer chromatography |
| Prep-TLC | Preparative thin layer chromatography |
| MOMO | Methoxymethoxyl |
| TIPS | Triisopropylsilyl |
| IPA | Isopropyl alcohol |
| cataCXium A | Methanesulfonato(diadamantyl-n-butylphosphino)- |
| Pd G3 | 2′-amino-1,1′-biphenyl-2-yl)palladium(II) |
| 4A MS | 4A Molecular sieve |

### Preparation of the Intermediates

The intermediates

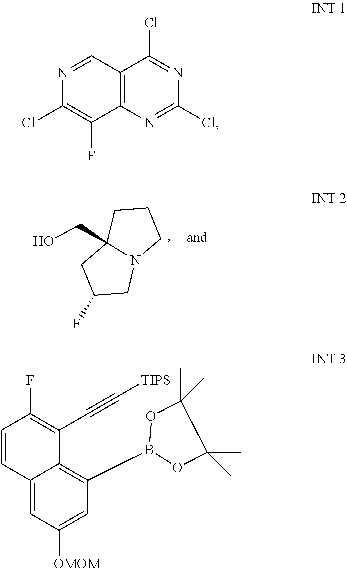

were synthesized using conventional preparation method.

### Example 1

### 4-(4-(cyclopropyl(methyl)amino)-8-fluoro-2-(((2R,7aS)-2-fluorotetrahydro-1H-pyrrolizin-7a(5H)-yl) methoxy)pyrido[4,3-d]pyrimidin-7-yl)-5-ethynyl-6-fluoronaphthalen-2-ol (“Compound 1”)

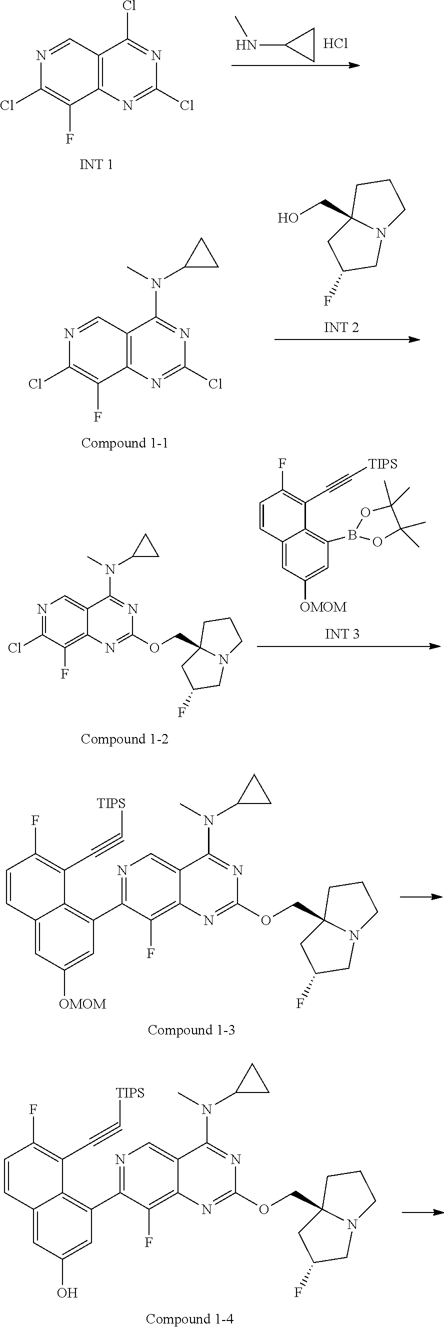

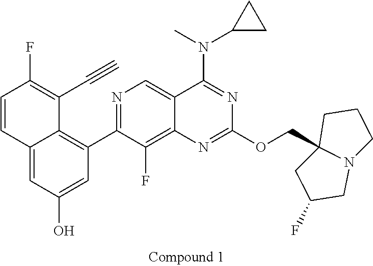 [SMILES: C#Cc1c(F)ccc2cc(O)cc(-c3ncc4c(N(C)C5CC5)nc(OC[C@@]56CCCN5C[C@H](F)C6)nc4c3F)c12]

To a solution of 2,4,7-trichloro-8-fluoropyrido[4,3-d]pyrimidine (4.88 g, 19.3298 mmol) and N,N-diisopropylethylamine (7.45 g, 57.6436 mmol) in DCM (70 mL) was added N-methylcyclopropanamine hydrochloride (2.01 g, 18.6835 mmol) at 0° C., then the mixture was stirred at room temperature for 2 h. The solution was diluted with 10% aqueous NaHCO₃ solution (100 mL), and the organic layer was washed with saturated aqueous NaCl solution, dried over anhydrous Na₂SO₄ and concentrated in vacuum. The residue was slurried with 70 ml solution (EA:Hex=1:6) to give Compound 1-1 (25163 mg, 17.9820 mmol, 93.0276% yield). MS m/z: 287 [M+H]⁺.

A solution of Compound 1-1 (5.16 g, 17.9716 mmol), INT 2 (3.84 g, 24.1205 mmol) and KF (3.29 g, 56.6297 mmol) in DMSO (150 mL) was stirred at 100° C. for 20 h under nitrogen atmosphere. The mixture was allowed to cool to room temperature, diluted with saturated aqueous NaHCO₃ solution (150 mL) and extracted with EA (150 mL). The organic layer was washed with 200 ml aqueous NaCl solution, then dried over anhydrous Na₂SO₄ and concentrated in vacuum. The residue was slurried with 70 ml solution (EA:Hex=1:6) to give Compound 1-2 (5.51 g, 13.4436 mmol, 74.8049% yield). MS m/z: 410 [M+H]⁺.

To a solution of Compound 1-2 (5.51 g, 13.4436 mmol) in toluene (250 mL), INT 3 (8.95 g, 17.4622 mmol), cataCXium A Pd G3 (1.46 g, 2.0048 mmol), potassium phosphate (13.23 g, 40.6054 mmol) and water (50 mL) were added. The reaction mixture was stirred at 100° C. for 20 hours under nitrogen atmosphere. The mixture was allowed to cool to room temperature, diluted with saturated aqueous NaHCO₃ solution (150 mL) and extracted with EA (150 mL). The organic layer was washed with 200 ml aqueous NaCl solution then dried over anhydrous Na₂SO₄ and concentrated in vacuum. The residue was purified by silica column (method: DCM:MeOH=1:0˜30:1) to give Compound 1-3 (8.18 g, 10.7635 mmol, 80.0643% yield). MS m/z: 760 [M+H]⁺. To a solution of Compound 1-3 (8.18 g, 10.7635 mmol) in DCM (100 mL) was added 4 M HCl in dioxane (15 ml) and stirred at room temperature for 1 h. The solution was diluted with 10% aqueous NaHCO₃ solution (150 mL). The organic layer was washed with saturated aqueous NaCl solution, dried over anhydrous Na₂SO₄ and concentrated in vacuum to give crude Compound 1-4 (10.6 g, 14.8061 mmol, 137.5581% yield). MS m/z: 716 [M+H]⁺.

To a solution of Compound 1-4 (10.6 g, 14.8061 mmol) in DMF (100 mL) was added CsF (11.81 g, 77.7468 mmol). The reaction mixture was stirred for 20 hours at 40° C. under nitrogen atmosphere. The solution was diluted with water (80 mL) and extracted with EA (80 mL). The organic layer was dried over anhydrous Na₂SO₄ and concentrated under vacuum. The residue was purified by Prep-HPLC (C18 column, A: 0.05% TFA in water, B: CH₃CN, Gradient: 15% B to 15% B in 2 min, 15% B to 40% B in 28 min, 40% B to 55% B in 30 min at a flow rate of 200 mL/min, 230 n). The eluent was adjusted to pH=8 with saturated NaHCO₃. The acetonitrile in the eluent was concentrated. The resulting aqueous layer was extracted with DCM and the organic layer was washed with water, then the organic layer was dried, concentrated and lyophilized to give Compound 1 (1922 mg, 3.4347 mmol, 23.1979% yield). MS m/z: 560 [M+H]⁺.

¹H NMR (400 MHz, CD₃OD) δ 9.57 (s, 1H), 7.85 (dd, J=8.9, 5.8 Hz, 1H), 7.38-7.27 (m, 2H), 7.22 (s, 1H), 5.29 (d, J=54.0 Hz, 1H), 4.26 (ddd, J=30.4, 10.5, 4.3 Hz, 2H), 3.64-3.49 (m, 1H), 3.45 (s, 3H), 3.40 (d, J=4.7 Hz, 1H), 3.29 (d, J=10.2 Hz, 3H), 3.25-3.14 (m, 2H), 3.06-2.93 (m, 1H), 2.22 (dd, J=14.5, 10.0 Hz, 1H), 2.16-2.06 (m, 1H), 2.03-1.92 (m, 2H), 1.91-1.79 (m, 1H), 1.14 (d, J=6.5 Hz, 2H), 0.99-0.79 (m, 2H).

### Example 2

### 4-(4-(cyclopropyl(methyl)amino)-8-fluoro-2-(((2R,7aS)-2-fluorotetrahydro-1H-pyrrolizin-7a(5H)-yl) methoxy)-5-methoxypyrido[4,3-d]pyrimidin-7-yl)-5-ethynyl-6-fluoronaphthalen-2-ol (“Compound 2”)

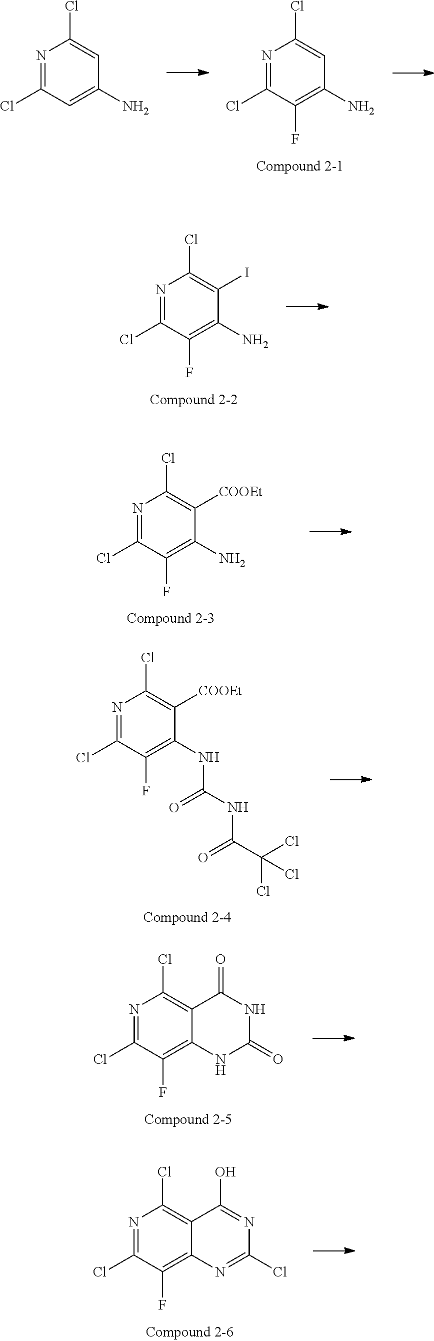

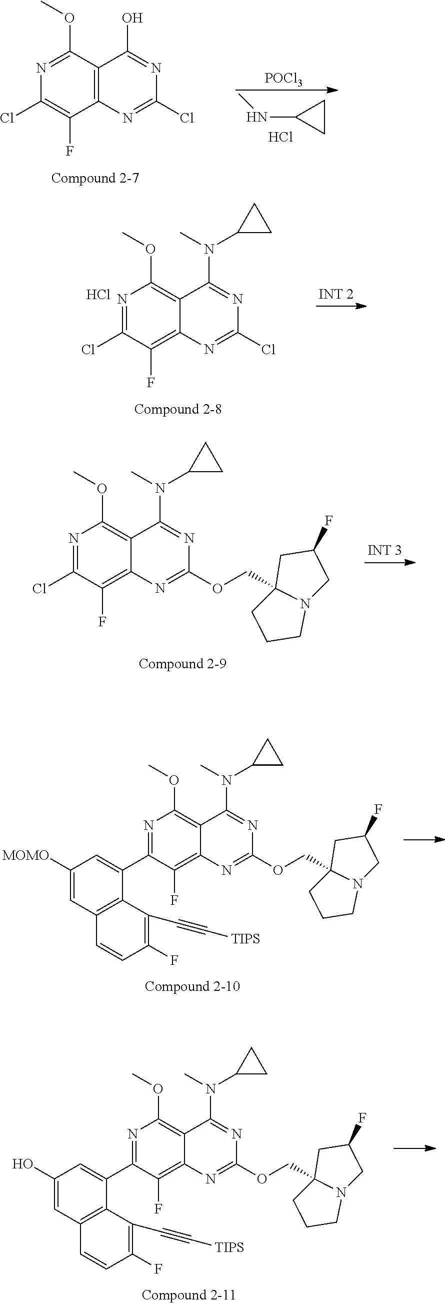

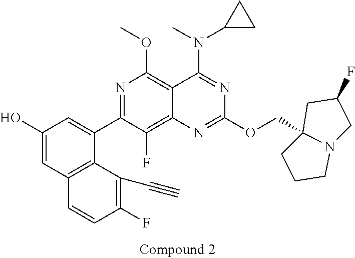 [SMILES: C#Cc1c(F)ccc2cc(O)cc(-c3nc(OC)c4c(N(C)C5CC5)nc(OC[C@@]56CCCN5C[C@H](F)C6)nc4c3F)c12]

A mixture of 2,6-dichloropyridin-4-amine (35.7 g, 219.0 mmol), 1-(chloromethyl)-4-fluoro-1,4-diazoniabicyclo[2.2.2]octane ditetrafluoroborate (93.1 g, 262.8 mmol) in DMF (357 mL) and CH₃CN (357 mL) was stirred at 80° C. for 6 hours. The reaction mixture was quenched by water (400 mL) and extracted with DCM (400 mL×3). The organic layers were combined, dried over anhydrous Na₂SO₄, filtered and concentrated under reduced pressure. The residue was purified by silica gel column (eluting with petroleum ether:EtOAc=30:1, v/v) to give Compound 2-1 (12.6 g, purity: about 50%). MS (ESI, m/z): 181 [M+H]⁺.

A mixture of Compound 2-1 (2.0 g, 11.05 mmol), NIS (2.98 g, 13.26 mmol) and p-toluenesulfonic acid monohydrate (105 mg, 0.55 mmol) in CH₃CN (8.4 mL) was stirred at 70° C. for 4 hours under nitrogen atmosphere. The reaction mixture was quenched by water (20 mL) and extracted with EtOAc (20 mL×3). The organic layers were combined, dried over anhydrous Na₂SO₄, filtered and concentrated under reduced pressure.

The residue was purified by silica gel column (eluting with petroleum ether:EtOAc=50:1-20:1, v/v) to give Compound 2-2 (3.6 g). MS (ESI, m/z): 307 [M+H]⁺.

A mixture of Compound 2-2 (1.0 g, 3.26 mmol), Pd(PPh₃)₂Cl₂ (229 mg, 0.33 mmol) and Et₃N (1.19 g, 11.77 mmol) in EtOH (17.0 mL) was stirred at 80° C. for 20 hours under carbon monoxide atmosphere (1.5 MPa) in a sealed tube. The reaction mixture was filtered and the filtrate was concentrated under reduced pressure. The residue was purified by silica gel column to give Compound 2-3 (1.2 g). MS (ESI, m/z): 253 [M+H]⁺.

A mixture of Compound 2-3 (800 mg, 3.16 mmol), trichloroacetyl isocyanate (714 mg, 3.79 mmol) in THF (8 mL) was stirred at room temperature for 1 h. The reaction mixture was concentrated under reduced pressure. The residue was triturated with MTBE to give Compound 2-4 (880 mg). MS (ESI, m/z): 442 [M+H]⁺.

A mixture of Compound 2-4 (780 mg, 1.77 mmol), NH₃/MeOH (1.26 mL, 7M, 8.85 mmol) and MeOH (7.8 mL) was stirred at room temperature for 1 h. The reaction mixture was concentrated under reduced pressure. The residue was triturated with MTBE to give Compound 2-5 (550 mg). MS (ESI, m/z): 250 [M+H]⁺.

A mixture of Compound 2-5 (375 mg, 1.50 mmol), DIPEA (595 mg, 4.60 mmol) and POCl₃ (15 mL) was stirred at 105° C. for 17 hours. The reaction mixture was concentrated under reduced pressure. The residue was diluted with 1,4-dioxane (5 mL) and the resulting solution was added dropwise to aq. K₂CO₃ (20%, 30 mL).

The mixture was stirred for 2 hours at RT and the pH of the mixture was adjusted to 2˜3. Then, the mixture was filtered and the filter cake was collected and dried to give Compound 2-6 (344 mg). MS (ESI, m/z): 268 [M+H]⁺.

To a solution of Compound 2-6 (201.6 mg, 0.75 mmol) in dry THF (5 mL) was added sodium methanolate (111.2 mg, 2.06 mmol) and then stirred at room temperature for 15 hours. Upon completion, the mixture was adjusted to pH 5-6 with 5% citric acid and extracted with EtOAc twice. The combined organic layers were washed with brine, dried over anhydrous Na₂SO₄, filtered and concentrated under reduced pressure to give Compound 2-7 (203 mg, crude). MS: m/z 264 [M+1]⁺.

To a solution of Compound 2-7 (313 mg, 1.19 mmol) in toluene (10 mL) was added DIEA (0.5 mL) and POCl₃ (1 mL) and stirred for 3.5 h at 100° C. The reaction mixture was concentrated under vacuum. The residue was dissolved in DCM (10 mL) and the resulting mixture was added to a solution of N-methylcyclopropanamine hydrochloride (128 mg, 1.19 mmol) and DIEA (491 mg, 3.80 mmol) in DUM (10 mL) at −5° C. The mixture was stirred at room temperature for 1.5 h. The reaction was diluted with H₂O (30 mL) and extracted with DCM (30 mL). The organic layer was dried over anhydrous Na₂SO₄ and concentrated under vacuum. The residue was purified by Pre-TLC to give Compound 2-8 (235 mg, 0.74 mmol). MS (ESI, m/z): 317 [M+H]⁺.

To a solution of Compound 2-8 (235 mg, 0.74 mmol) and INT 2 (132 mg, 0.83 mmol) in DMSO (10 mL) was added KF (145 mg, 2.50 mmol). The reaction mixture was stirred at 95° C. for 16 hours under nitrogen atmosphere. The resulting mixture was quenched by water (30 mL) and extracted with EA (2×30 mL). The organic layer was washed with brine (30 mL), dried over anhydrous Na₂SO₄ and concentrated in vacuum. The residue was purified by Pre-TLC to give Compound 2-9 (124 mg, 0.28 mmol). MS (ESI, m/z): 440 [M+H]⁺.

A solution of Compound 2-9 (66 mg, 0.15 mmol), INT 3 (94 mg, 0.18 mmol), cataCXium A Pd G3 (32 mg, 43.94 μmol), Cs₂CO₃ (100 mg, 0.31 mmol) in toluene (4 mL) and water (1 mL) was stirred at 100° C. for 18 hours under nitrogen atmosphere. The reaction mixture was diluted with EA (30 mL) and washed with water (2×20 mL). The organic layer was dried over anhydrous Na₂SO₄ and concentrated under vacuum. The residue was purified by Pre-TLC to give Compound 2-10 (104 mg, 131.65 μmol). MS (ESI, m/z): 790 [M+H]⁺.

To a solution of Compound 2-10 (104 mg, 131.65 μmol) in CH₃CN (3 mL) was added HCl (1 mL, 4 M in 1,4-dioxane). The reaction mixture was stirred at room temperature for 1 hour. The resulting mixture was quenched by saturated aqueous NaHCO₃ solution (20 mL) and extracted with EA (2×30 mL). The organic layer was dried over anhydrous Na₂SO₄ and concentrated under reduced pressure to give Compound 2-11 (109 mg, 146.12 μmol). MS (ESI, m/z): 746 [M+H]⁺.

To a solution of Compound 2-11 (109 mg, 146.12 μmol) in DMF (3 mL) was added CsF (0.40 g, 2.63 mmol). The reaction mixture was stirred at 40° C. for 16 hours. The mixture was diluted with saturated aqueous NaHCO₃ solution (20 mL) and extracted with EA (2×20 mL). The organic layers were combined, dried over anhydrous Na₂SO₄ and concentrated under vacuum. The residue was purified by Prep-HPLC (C18 column, A: 0.1% TFA in water, B: CH₃CN, Gradient: 15% B to 40% B in 36 min at a flow rate of 60 mL/min, 240 nm).

The acetonitrile in the eluent was concentrated. The resulting aqueous layer was added NaHCO₃ (30 mL), extracted with EA (30 mL×2) and the organic layer was concentrated under reduced pressure to give Compound 2 (17.7 mg, 30.02 μmol). MS (ESI, m/z): 590 [M+H]⁺.

### Example 3

### 5-ethynyl-6-fluoro-4-(8-fluoro-4-((trans-2-fluorocyclopropyl)(methyl)amino)-2-(((2R,7aS)-2-fluorotetrahydro-1H-pyrrolizin-7a(5H)-yl)methoxy)pyrido[4,3-d]pyrimidin-7-yl)naphthalen-2-ol 2,2,2-trifluoroacetic acid (“Compound 3 (TFA salt)”)

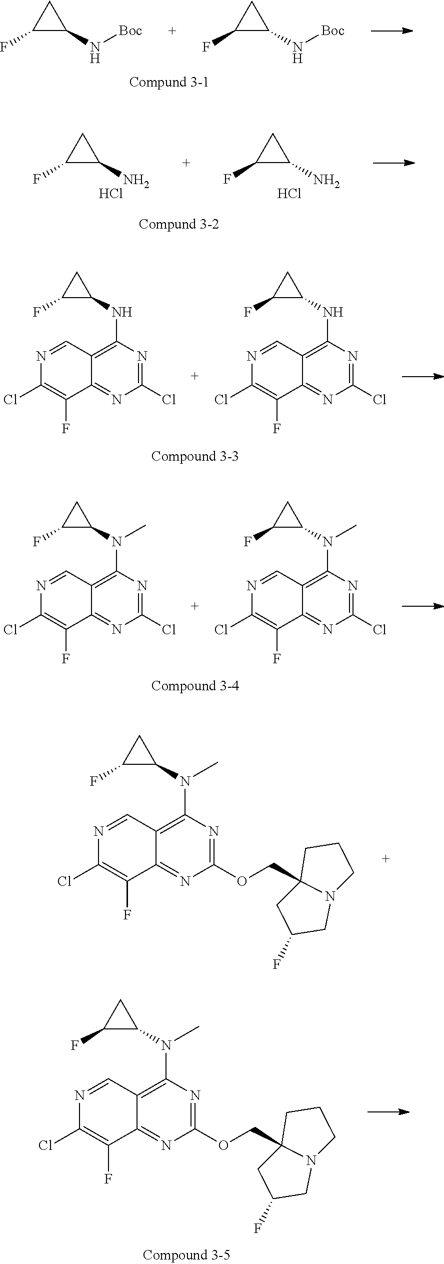

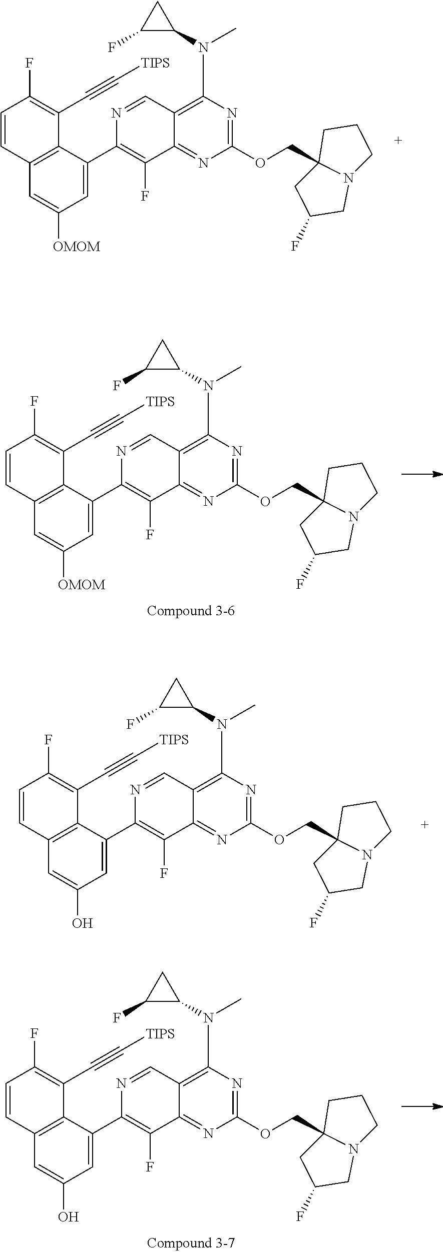

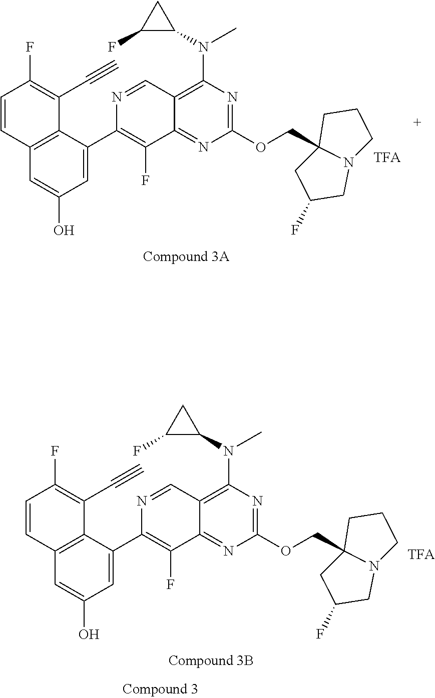 [SMILES: C#Cc1c(F)ccc2cc(O)cc(-c3ncc4c(N(C)[C@H]5C[C@@H]5F)nc(OC[C@@]56CCCN5C[C@H](F)C6)nc4c3F)c12 ; C#Cc1c(F)ccc2cc(O)cc(-c3ncc4c(N(C)[C@@H]5C[C@H]5F)nc(OC[C@@]56CCCN5C[C@H](F)C6)nc4c3F)c12]

To a solution of Compound 3-1 (trans-tert-butyl(2-fluorocyclopropyl)carbamate, 601 mg, 3.43 mmol) in EA (10 mL) was added HCl (6 mL, 4 M in EA). The reaction mixture was stirred at room temperature overnight under nitrogen atmosphere and concentrated under reduced pressure to give Compound 3-2 (408 mg, crude, HCl salt). MS m/z: 117 [M+H+41]⁺.

To a solution of 2,4,7-trichloro-8-fluoropyrido[4,3-d]pyrimidine (0.68 g, 2.69 mmol), DIEA (1.42 g, 10.99 mmol) in DCM (8 mL) was added Compound 3-2 (408 mg) at 0° C. The mixture was stirred at room temperature for 1 h, and then diluted with DCM (20 mL). The organic layer was washed with brine (20 mL×2), dried over anhydrous Na₂SO₄ and concentrated under reduced pressure. The residue was purified by Pre-TLC (MeOH:DCM=1:20, v/v) to give Compound 3-3 (413 mg, 1.42 mmol). MS m/z: 291/293 [M+H]⁺.

To a solution of Compound 3-3 (341 mg, 1.17 mmol), CH₃I (692 mg, 4.88 mmol) in DMF (2 mL) was added NaH (71 mg, 1.78 mmol, 60% content). The mixture was stirred at room temperature for 4 h, quenched with saturated NH₄Cl aqueous solution (2 mL), extracted with EA (30 mL) and washed with brine (20 mL×2), dried over anhydrous Na₂SO₄ and concentrated under reduced pressure. The residue was purified by Pre-TLC (EA:Hex=2:1, v/v) to give Compound 3-4 (271 mg, 0.89 mmol). MS m/z: 305/307 [M+H]⁺.

A solution of Compound 3-4 (271 mg, 0.89 mmol), INT 2 (217 mg, 1.36 mmol) and KF (166 mg, 2.86 mmol) in DMSO (6 mL) was stirred at 90° C. for 20 h under nitrogen atmosphere. The mixture was allowed to cool to room temperature and extracted with EA (30 mL). The organic layer was washed with brine (30 mL×2), dried over anhydrous Na₂SO₄ and concentrated under reduced pressure. The residue was purified by Pre-TLC (DCM:MeOH=15:1, v/v) to give Compound 3-5 (139 mg, 0.325 mmol). MS m/z: 428 [M+H]⁺.

To a solution of Compound 3-5 (139 mg, 0.325 mmol), INT 3 (253 mg, 0.494 mmol) in toluene (5 mL) and water (1 mL) was added Cs₂CO₃ (322 mg, 0.988 mmol) and cataCXium A Pd G3 (37 mg, 0.508 mmol). The reaction mixture was stirred at 100° C. overnight under nitrogen atmosphere. The reaction was filtered and the filtrate was concentrated under reduced pressure. The residue was purified by Pre-TLC (DCM:MeOH=15:1, v/v) to give Compound 3-6 as a brown solid (203 mg, 0.261 mmol). MS m/z: 778 [M+H]⁺.

A solution of Compound 3-6 (203 mg, 0.261 mmol) and HCl (4 M in 1,4-dioxane, 1 mL) in ACN (4 mL) was stirred at RT for 1 h. The solution was diluted with saturated NaHCO₃ aqueous solution (20 mL) and extracted with EA (30 mL). The organic layer was washed with brine (30 mL), dried over anhydrous Na₂SO₄ and concentrated under reduced pressure to give Compound 3-7 (crude, 210 mg, 0.286 mmol). MS m/z: 734 [M+H]⁺.

A solution of Compound 3-7 (210 mg, 0.286 mmol) and CsF (0.77 g, 5.07 mmol) in DMF (5 mL) was stirred at 40° C. for 2 h under nitrogen atmosphere. The mixture was diluted with water (10 mL) and extracted with EA (20 mL). The collected organic layer was dried over anhydrous Na₂SO₄ and concentrated under reduced pressure. The residue was purified by Prep-HPLC (C18 column, A: 0.1% TFA in water, B: CH₃CN, Gradient: 15% B to 45% B in 45 min at a flow rate of 60 mL/min, 235 nm) to give Compound 3 (TFA salt) containing Compound 3A (TFA salt) and Compound 3B (TFA salt) (118.3 mg, 0.171 mmol). MS m/z: 578 [M+H]⁺.

### Example 4

### 5-ethynyl-6-fluoro-4-(8-fluoro-4-(((1S,2S)-2-fluorocyclopropyl)(methyl)amino)-2-(((2R,7aS)-2-fluoro tetrahydro-1H-pyrrolizin-7a(5H)-yl)methoxy)pyrido[4,3-d]pyrimidin-7-yl)naphthalen-2-ol 2,2,2-trifluoroacetic acid (“Compound 3A (TFA salt)”)

### 5-ethynyl-6-fluoro-4-(8-fluoro-4-(((1R,2R)-2-fluorocyclopropyl)(methyl)amino)-2-(((2R,7aS)-2-fluorotetrahydro-1H-pyrrolizin-7a(5H)-yl)methoxy)pyrido[4,3-d]pyrimidin-7-yl)naphthalen-2-ol 2,2,2-trifluoroacetic acid (“Compound 3B (TFA salt)”)

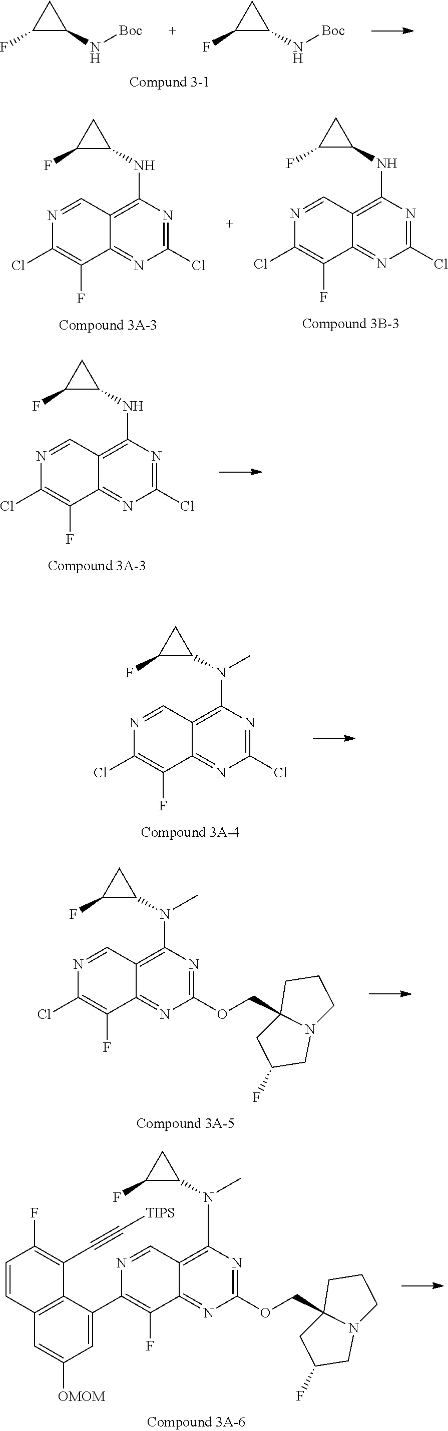

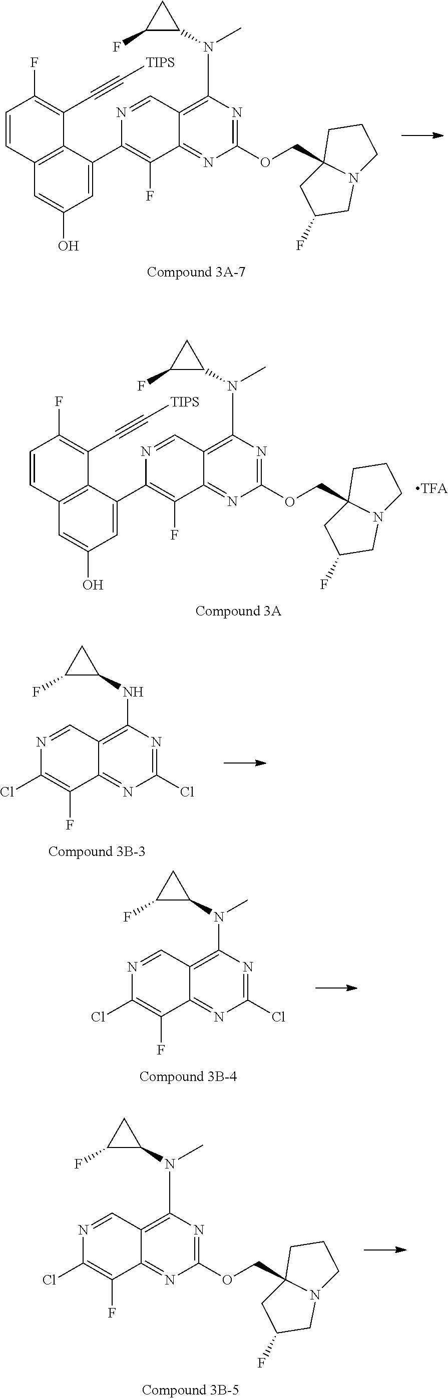

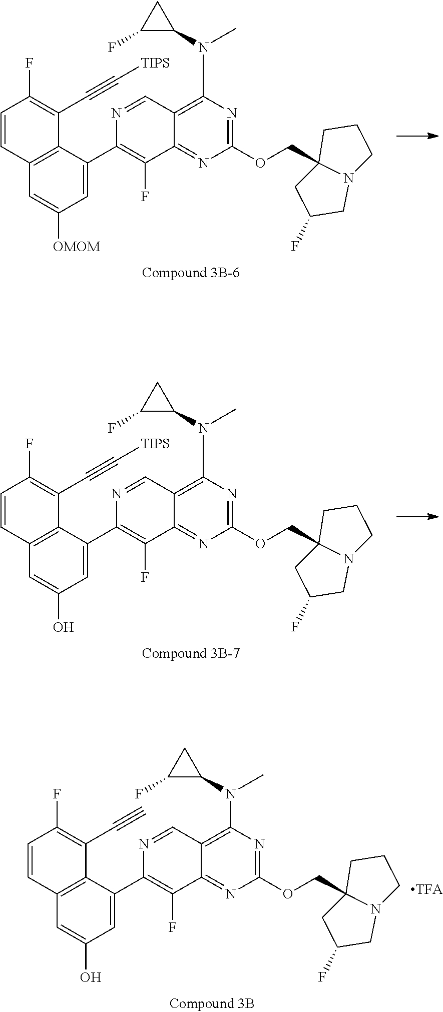

To a solution of Compound 3-1 (trans-ter-butyl (2-fluorocyclopropyl)-carbamate, 10 g, 5.8-ml) in for 4 h, and concentrated under reduced pressure to give an off white solid. The off white solid was added to a solution of 2,4,7-trichloro-8-fluoropyrido[4,3-d]pyrimidine (14.41 g, 57.08 mmol) and DIEA (19.63 g, 151.89 mmol) in DCM (100 mL) at 0° C. The mixture was stirred at room temperature for 1 h and diluted with water (80 mL). The collected organic layer was washed with brine twice (20 mL), dried over anhydrous Na₂SO₄ and concentrated under reduced pressure. The residue was stirred with Hex:EA=15:1 (160 ml) and filtered to give a brown solid (17.68 g, 60.74 mmol). The solid was separated by Prep-HPLC-Gilson with the following conditions: Column, CHIRAL ART Cellulose-SC column (2 cm×25 cm, 5 m); mobile phase, Hex/EtOH (50:50, v/v); Flowing rate: 20 ml/min. This results in Compound 3A-3 (7.76 g, 26.66 mmol, the first eluting isomer, Retention Time 3.831 min) and Compound 3B-3 (6.95 g, 23.88 mmol, the second eluting isomer, Retention Time 5.243 min). MS m/z: 291/293 [M+H]⁺.

To a solution of Compound 3A-3 (244 mg, 0.84 mmol) and CH₃I (507 mg, 3.57 mmol) in DMF (4 mL) was added NaH (45 mg, 1.13 mmol, 60% content). The mixture was stirred at room temperature for 22 h, quenched with water (30 mL), extracted with EA (30 mL), washed with brine twice (20 mL), dried over anhydrous Na₂SO₄ and concentrated under reduced pressure to give Compound 3A-4 (crude, 237 mg, 0.78 mmol). MS m/z: 305/307 [M+H]⁺.

To a solution of Compound 3A-4 (237 mg, 0.78 mmol) and INT 2 (145 mg, 0.91 mmol) in THF (5 mL) was added t-BuONa (98 mg, 1.02 mmol) in portions at −10° C. The mixture was stirred at −10° C. for 2.5 h. The mixture was quenched with water (30 mL) and extracted with EA (30 mL). The collected organic layer was washed with brine (30 mL×2), dried over anhydrous Na₂SO₄ and concentrated under reduced pressure. The residue was purified by Pre-TLC (DCM:MeOH=15:1, v/v) to give Compound 3A-5 (154 mg, 0.36 mmol). MS m/z: 428 [M+H]⁺.

To a mixture of Compound 3A-5 (154 mg, 0.36 mmol), INT 3 (228 mg, 0.44 mmol) in toluene (10 mL) and water (2.5 mL) were added Cs₂CO₃ (240 mg, 0.74 mmol) and cataCXium A Pd G3 (36 mg, 0.049 mmol). The reaction mixture was stirred at 100° C. overnight under nitrogen atmosphere. The reaction mixture was filtered and the filtrate was concentrated under reduced pressure. The residue was purified by Pre-TLC (DCM:MeOH=15:1, v/v) to give Compound 3A-6 (186 mg, 0.24 mmol). MS m/z: 778 [M+H]⁺.

A solution of Compound 3A-6 (186 mg, 0.24 mmol) and HCl (4 M in dioxane, 1.5 mL) in ACN (4.5 mL) was stirred at RT for 1 h. The solution was diluted with saturated NaHCO₃ aqueous solution (20 mL) and extracted with EA (30 mL). The collected organic layer was washed with brine (30 mL), dried over anhydrous Na₂SO₄ and concentrated under reduced pressure to give Compound 3A-7 (crude, 180 mg, 0.25 mmol). MS m/z: 734 [M+H]⁺.

A mixture of Compound 3A-7 (180 mg, 0.25 mmol) and CsF (0.51 g, 3.36 mmol) in DMF (5 mL) was stirred at 45° C. for 2 h. The mixture was diluted with water (20 mL) and extracted with EA (20 mL). The collected organic layer was dried over anhydrous Na₂SO₄ and concentrated under reduced pressure. The residue was purified by Prep-HPLC (C18 column, A: 0.1% TFA in water, B: CH₃CN, Gradient: 15% B to 43% B in 30 min at a flow rate of 60 mL/min, 230 nm) to give Compound 3A (TFA salt) (112.6 mg, 0.16 mmol). MS m/z: 578 [M+H]⁺.

¹H NMR (400 MHz, CD₃OD) δ 9.58 (d, 1H), 7.97-7.83 (m, 1H), 7.46-7.32 (m, 2H), 7.26 (s, 1H), 5.58 (d, 1H), 4.96-4.65 (m, 3H), 4.20-3.75 (m, 4H), 3.59-3.36 (m, 5H), 2.81-2.52 (m, 2H), 2.51-2.28 (m, 3H), 2.19 (s, 1H), 1.78 (d, 1H), 1.48-1.26 (m, 1H).

To a solution of Compound 3B-3 (246 mg, 0.85 mmol) and CH₃I (510 mg, 3.59 mmol) in DMF (4 mL) was added NaH (45 mg, 1.13 mmol, 60% content). The mixture was stirred at room temperature for 22 h, quenched with water (30 mL), extracted with EA (30 mL), washed with brine twice (20 mL), dried over anhydrous Na₂SO₄ and concentrated under reduced pressure to give Compound 3B-4 (crude, 271 mg, 0.89 mmol). MS m/z: 305/307 [M+H]⁺.

To a solution of Compound 3B-4 (271 mg, 0.89 mmol) and INT 2 (158 mg, 0.99 mmol) in THF (5 mL) was added t-BuONa (110 mg, 1.14 mmol) in portions at −10° C. The mixture was stirred at −10° C. for 2.5 h.

The mixture was quenched with water (30 ml) and extracted with EA (30 mL). The collected organic layer was washed with brine (30 mL×2), dried over anhydrous Na₂SO₄ and concentrated under reduced pressure. The residue was purified by Pre-TLC (DCM:MeOH=15:1, v/v) to give Compound 3B-5 (150 mg, 0.35 mmol). MS m/z: 428 [M+H]⁺.

To a solution of Compound 3B-5 (150 mg, 0.35 mmol), INT 3 (231 mg, 0.45 mmol) in toluene (10 mL) and water (2.5 mL) were added Cs₂CO₃ (250 mg, 0.77 mmol) and cataCXium A Pd G3 (39 mg, 0.053 mmol).

The reaction mixture was stirred at 100° C. for overnight under nitrogen atmosphere. The reaction mixture was filtered and the filtrate was concentrated under reduced pressure. The residue was purified by Pre-TLC (DCM:MeOH=15:1, v/v) to give Compound 3B-6 (163 mg, 0.21 mmol). MS m/z: 778 [M+H]⁺.

A solution of Compound 3B-6 (163 mg, 0.21 mmol) and HCl (4 M in dioxane, 1.5 mL) in ACN (4.5 mL) was stirred at room temperature for 1 h. The solution was diluted with saturated NaHCO₃ aqueous solution (20 mL) and extracted with EA (30 mL). The collected organic layer was washed with brine (30 mL), dried over anhydrous Na₂SO₄ and concentrated under reduced pressure to give Compound 3B-7 (crude, 151 mg, 0.21 mmol). MS m/z: 734 [M+H]⁺.

A solution of Compound 3B-7 (151 mg, 0.21 mmol) and CsF (0.77 g, 5.07 mmol) in DMF (5 mL) was stirred at 45° C. for 2 h. The mixture was diluted with water (20 mL) and extracted with EA (20 mL). The collected organic layer was dried over anhydrous Na₂SO₄ and concentrated under reduced pressure. The residue was purified by Prep-HPLC (C18 column, A: 0.1% TFA in water, B: CH₃CN, Gradient: 15% B to 43% B in 33 min at a flow rate of 60 mL/min, 230 n) to give Compound 3B (TFA salt) (89.9 mg, 0.13 mmol). MS m/z: 578 [M+H]⁺.

¹H NMR (400 MHz, CD₃OD) δ 9.59 (d, 1H), 7.98-7.83 (m, 1H), 7.50-7.32 (m, 2H), 7.28 (s, 1H), 5.58 (d, 1H), 4.96-4.60 (m, 3H), 4.15-3.78 (m, 4H), 3.60-3.39 (m, 5H), 2.83-2.52 (m, 2H), 2.51-2.30 (m, 3H), 2.19 (s, 1H), 1.79 (d, 1H), 1.47-1.25 (m, 1H).

### Example 5

### 5-ethynyl-6-fluoro-4-(8-fluoro-4-(((1S,2S)-2-fluorocyclopropyl)(methyl)amino)-2-(((2R,7aS)-2-fluoro tetrahydro-1H-pyrrolizin-7a(5H)-yl)methoxy)pyrido[4,3-d]pyrimidin-7-yl)naphthalen-2-ol (“Compound 3A”)

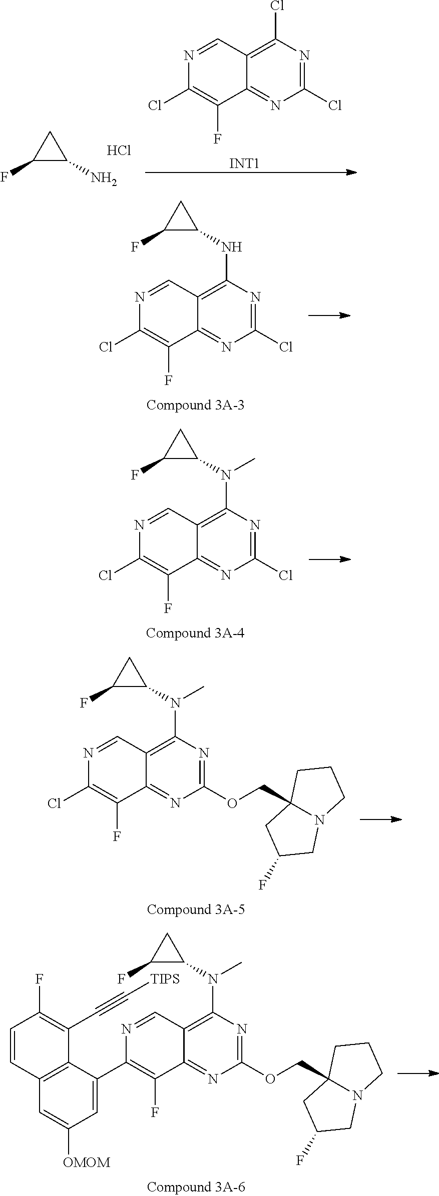

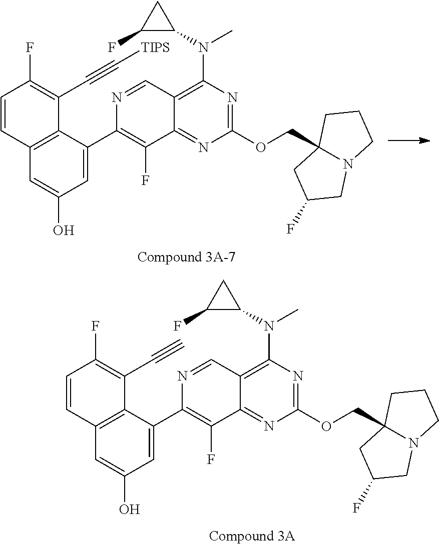

To a solution of 2,4,7-trichloro-8-fluoropyrido[4,3-d]pyrimidine (INT-1, 2.09 g, 8.28 mmol) and DIEA (2.16 g, 16.71 mmol) in DCM (30 mL) was added (1S,2S)-2-fluorocyclopropan-1-amine hydrochloride (0.93 g, 8.33 mmol) at 0° C. The mixture was stirred at room temperature for 1 h, diluted with DCM (20 mL), washed with water (20 mL×2), dried over anhydrous Na₂SO₄ and concentrated under reduced pressure to give Compound 3A-3 (2.40 g, 8.25 mmol, 98.99% yield). MS m/z: 291/293 [M+H]⁺.

To a solution of Compound 3A-3 (2.57 g, 8.84 mmol) and CH₃I (4.84 g, 34.10 mmol) in DMF (25 mL) was added NaH (0.44 g, 11.00 mmol, 60% content). The mixture was stirred at room temperature for 5 h, quenched with saturated NH₄Cl (20 mL) aqueous solution, extracted with EA (100 mL) and washed with brine (50 mL×2), dried over anhydrous Na₂SO₄ and concentrated under reduced pressure. The residue was slurried with (50 mL, EA:Hex=1:10, v/v) to give Compound 3A-4 (2.25 g, 6.64 mmol, 75.05% yield). MS m/z: 305/307 [M+H]⁺.

To a solution of Compound 3A-4 (2.25 g, 6.64 mmol) and INT 2 (1.17 g, 7.35 mmol) in THF (30 mL) was added t-BuONa (0.85 g, 8.84 mmol) portion wise at −10° C. under nitrogen atmosphere. The mixture was stirred for 2 h, warmed to room temperature and extracted with EA (50 mL). The organic layer was washed with brine (30 mL×2), dried over anhydrous Na₂SO₄ and concentrated under reduced pressure. The residue was slurried with (50 mL, Hex:EA=9:1, v/v) to give Compound 3A-5 (2.44 g, 5.70 mmol, 77.33% yield). MS m/z: 428 [M+H]⁺.

To a solution of Compound 3A-5 (2.44 g, 5.70 mmol) and INT 3 (3.78 g, 7.38 mmol) in 1,4-dioxane (30 mL) and water (3 mL) were added Cs₂CO₃ (4.71 g, 14.46 mmol) and cataCXium A Pd G3 (0.42 g, 0.577 mmol). The reaction mixture was stirred at 100° C. overnight under nitrogen atmosphere. The reaction mixture was filtered and the filtrate was concentrated under reduced pressure. The residue was purified by silica gel column chromatography (DCM:MeOH=50:1 to 30:1, v/v) to give Compound 3A-6 (3.57 g, 4.59 mmol, 80.46% yield). MS m/z: 778 [M+H]⁺.

A solution of Compound 3A-6 (3.57 g, 4.59 mmol) and HCl (16 mL, 4 M in 1,4-dioxane) in ACN (45 mL) was stirred at room temperature for 1 h. The solution was diluted with saturated NaHCO₃ aqueous solution (30 mL) and extracted with EA (100 mL). The collected organic layer was washed with brine (50 mL×3), dried over anhydrous Na₂SO₄ and concentrated under reduced pressure to give Compound 3A-7 (crude, 3.57 g, 4.86 mmol, 106.00% yield). MS m/z: 734 [M+H]⁺.

A mixture of Compound 3A-7 (3.57 g, 4.86 mmol) and CsF (3.58 g, 23.57 mmol) in DMF (35 mL) was stirred at 40° C. for 2 h under nitrogen atmosphere. The solution was diluted with water (50 mL) and extracted with EA (100 mL). The collected organic layer was washed with brine (50 mL×3), dried over anhydrous Na₂SO₄ and concentrated under reduced pressure. The residue was slurried with ACN (12 mL) to give Compound 3A (2.05 g, 3.55 mmol, 73.01% yield). MS m/z: 578 [M+H]⁺.

¹H NMR (400 MHz, CD₃OD) δ 9.58 (d, 1H), 7.97-7.83 (m, 1H), 7.46-7.32 (m, 2H), 7.26 (s, 1H), 5.58 (d, 1H), 4.96-4.65 (m, 3H), 4.20-3.75 (m, 4H), 3.59-3.36 (m, 5H), 2.81-2.52 (m, 2H), 2.51-2.28 (m, 3H), 2.19 (s, 1H), 1.78 (d, 1H), 1.48-1.26 (m, 1H).

### Example 6

### 4-(4-(cyclopropyl(methyl-d3)amino)-8-fluoro-2-(((2R,7aS)-2-fluorotetrahydro-1H-pyrrolizin-7a(5H)-yl)methoxy)pyrido[4,3-d]pyrimidin-7-yl)-5-ethynyl-6-fluoronaphthalen-2-ol (“Compound 4”)

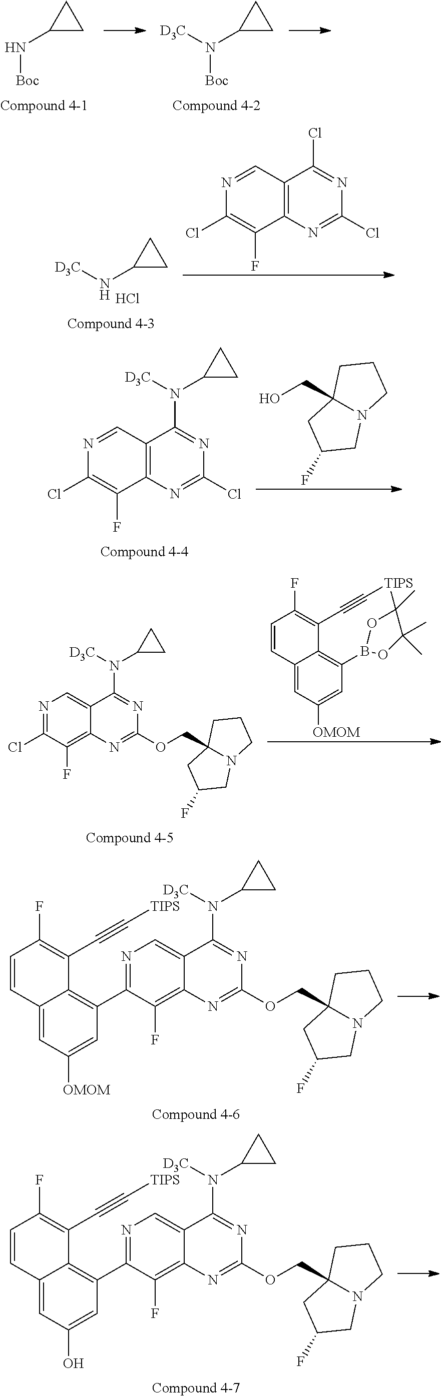

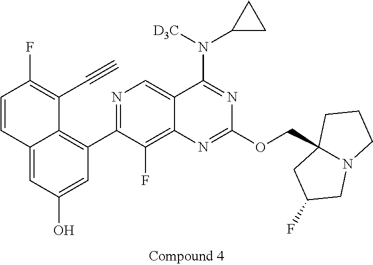 [SMILES: [2H]C([2H])([2H])N(c1nc(OC[C@@]23CCCN2C[C@H](F)C3)nc2c(F)c(-c3cc(O)cc4ccc(F)c(C#C)c34)ncc12)C1CC1]

NaH (0.92 g, 38.3370 mmol) was added into a solution of Compound 4-1 (3.09 g, 19.6553 mmol) in DMF (30 mL) at 0° C., stirred for 30 mins and then trideuterio(iodo)methane (3.37 g, 23.2483 mmol) was dropped into the system at 0° C. The solution was stirred at room temperature for 2 hours, quenched with water (30 mL) at room temperature, extracted with EA (30 mL×2). The organic layer was washed with NaCl aqueous solution (20 mL), dried over anhydrous Na₂SO₄ and concentrated in vacuum to give Compound 4-2 (3.71 g, 21.2907 mmol). MS m/z: 175 [M+H]⁺.

A solution of Compound 4-2 (3.71 g, 21.2906 mmol) and hydrogen chloride (4 M in dixoane, 10 mL) in DCM (10 mL) was stirred at room temperature for 2 h. then, more hydrogen chloride (4 M in dixoane, 10 mL) was added, and stirred at room temperature for 2 h. The solution was concentrated in vacuum to give Compound 4-3 (2.89 g, 26.1302 mmol). MS m/z: 75 [M+H]⁺.

Compound 4-3 (2.78 g, 25.1356 mmol) was dropped into the solution of 2,4,7-trichloro-8-fluoropyrido[4,3-d]pyrimidine (4.88 g, 19.3298 mmol) and N,N-diisopropylethylamine (7.56 g, 58.4947 mmol) in DCM (20 mL) at 0° C., then the solution was stirred at room temperature for 14 h. The solution was diluted with 10% citric acid aqueous solution (100 mL), then the organic layer was washed with NaCl aqueous solution (100 mL), dried over anhydrous Na₂SO₄ and concentrated in vacuum. The residue was slurried in 36 mL (EA:HEX=1:5) to give Compound 4-4 (5.27 g, 18.1637 mmol). MS m/z: 290 [M+H]⁺.

A solution of Compound 4-4 (5.27 g, 18.1637 mmol), INT 2 (4.33 g, 27.1984 mmol) and KF (3.29 g, 56.6297 mmol) in DMSO (150 mL) was stirred at 100° C. for 16 h under nitrogen atmosphere. The mixture was allowed to cool to room temperature, diluted with water (200 mL) and extracted with EA (200 mL). The organic layer was filtered and the filter cake was collected to give Compound 4-5 (2.79 g, 6.7574 mmol). MS m/z: 413 [M+H]⁺.

A solution of Compound 4-5 (2.73 g, 6.6121 mmol), INT 3 (5.23 g, 10.2042 mmol), cataCXium A Pd G3 (0.51 g, 700.2897 μmol) and cesium carbonate (6.69 g, 20.5329 mmol) in toluene (150 mL) and water (30 mL) was stirred at 100° C. for 20 hours under nitrogen atmosphere. The mixture was allowed to cool to room temperature, diluted with saturated NaHCO₃ aqueous solution (150 mL) and extracted with EA (150 mL). The organic layer was washed with aqueous NaCl solution (150 mL), then dried over anhydrous Na₂SO₄ and concentrated in vacuum. The residue was purified by silica column to give Compound 4-6 (3.91 g, 5.1246 mmol). MS m/z: 763 [M+H]⁺.

A solution of Compound 4-6 (3.91 g, 5.1246 mmol) and hydrogen chloride (4 M in dixoane, 15 mL) in DCM (50 mL) was stirred at room temperature for 4 h. The solution was diluted with 10% NaHCO₃ aqueous solution (20 mL) and extracted with DCM (20 mL). The organic layer was washed with saturated NaCl aqueous solution, dried over anhydrous Na₂SO₄ and concentrated in vacuum to give crude Compound 4-7 (4.47 g, 6.2175 mmol). MS m/z: 719 [M+H]⁺.

A solution of Compound 4-7 (5.75 g, 7.9979 mmol) and CsF (5.05 g, 33.2448 mmol) in DMF (100 mL) was stirred for 20 hours at 40° C. under nitrogen atmosphere. The solution was diluted with water (150 mL) and extracted with EA (150 mL). The organic layer was dried over anhydrous Na₂SO₄ and concentrated under vacuum. The residue was slurried with EA/Hex (15 mL/45 mL) to give Compound 4 (3.29 g, 5.8479 mmol). MS m/z: 563 [M+H]⁺.

¹H NMR (400 MHz, CD₃OD) δ 9.59 (s, 1H), 7.90-7.82 (m, 1H), 7.37-7.27 (m, 2H), 7.22 (d, J=2.4 Hz, 1H), 5.45-5.27 (m, 1H), 4.45-4.29 (m, 2H), 3.61-3.54 (m, 1H), 3.53-3.34 (m, 4H), 3.16-3.07 (m, 1H), 2.47-2.24 (m, 2H), 2.18 (d, J=8.5 Hz, 1H), 2.13-2.01 (m, 2H), 1.95 (dd, J=14.4, 7.4 Hz, 1H), 1.29 (s, 1H), 1.16 (d, J=6.7 Hz, 2H), 0.98-0.81 (m, 2H).

### Example 7

### 5-ethynyl-6-fluoro-4-(8-fluoro-4-((trans-2-fluorocyclopropyl)(methyl)amino)-2-(((2R,7aS)-2-methoxytetrahydro-1H-pyrrolizin-7a(5H)-yl)methoxy)pyrido[4,3-d]pyrimidin-7-yl)naphthalen-2-ol (“Compound 5”)

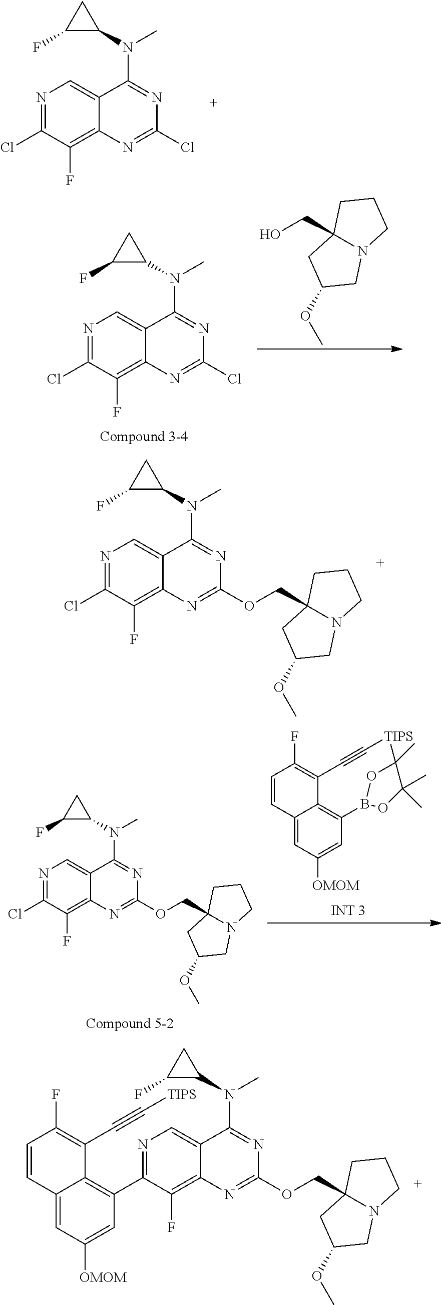

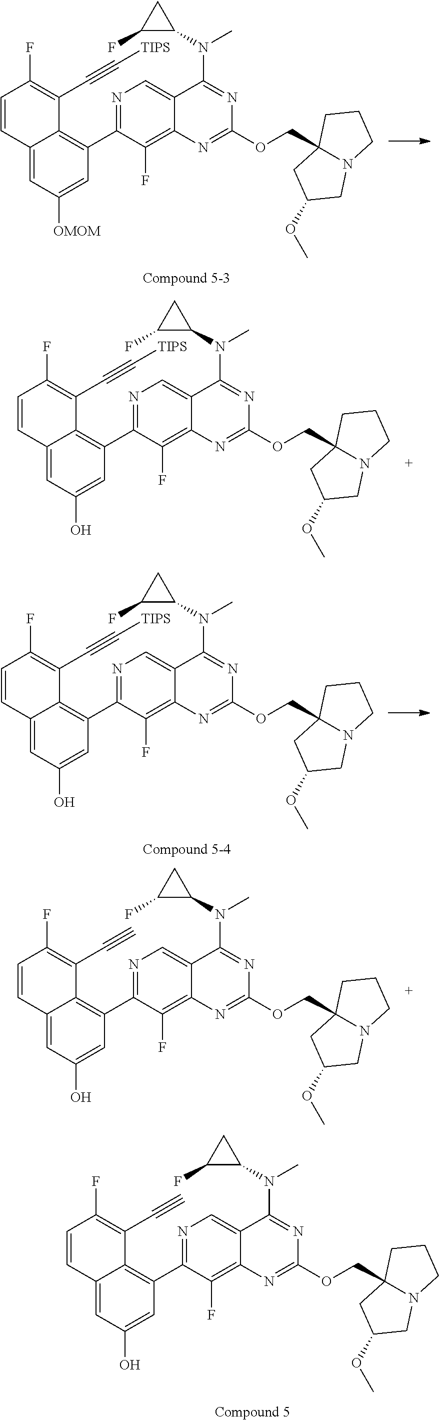

A solution of Compound 3-4 (165 mg, 540.7877 μmol), ((2R,7aS)-2-methoxytetrahydro-1H-pyrrolizin-7a(5H)-yl)methanol (99 mg, 578.1491 μmol) and KF (148 mg, 2.5475 mmol) in DMSO (5 mL) was stirred at 90° C. for 16 h under nitrogen atmosphere. The mixture was allowed to cool to room temperature, diluted with saturated NaHCO₃ aqueous solution (20 mL) and extracted with EA (30 mL×2). The organic layer was washed with NaCl aqueous solution, then dried over anhydrous Na₂SO₄ and concentrated in vacuum. The residue was purified by Pre-TLC to give Compound 5-2 (123 mg, 279.6181 μmol, 51.7075% yield). MS: m/z: 440 [M+H]⁺.

A solution of Compound 5-2 (123 mg, 279.6179 μmol), toluene (5 mL), INT 3 (224 mg, 437.0437 μmol), cataCXium A Pd G3 (42 mg, 57.6709 μmol), cesium carbonate (300 mg, 920.7569 μmol) and water (1 mL) was stirred at 100° C. for 18 hours under nitrogen atmosphere. The mixture was allowed to cool to room temperature, diluted with water (30 mL) and extracted with EA (2×30 mL). The organic layer was washed with NaCl aqueous solution, then dried over anhydrous Na₂SO₄ and concentrated in vacuum. The residue was purified by Pre-TLC to give Compound 5-3 (149 mg, 188.6080 μmol, 67.4520% yield). MS: m/z: 790 [M+H]⁺.

A solution of Compound 5-3 (48 mg, 60.7596 μmol) and HCl (4 M in 1,4-dioxane, 0.8 mL) in DCM (4 mL) was stirred at room temperature for 0.5 h. The solution was diluted with 10% NaHCO₃ aqueous solution (20 mL) and extracted with DCM (40 mL×2). The organic layer was washed with NaCl aqueous solution, dried over anhydrous Na₂SO₄ and concentrated in vacuum to give crude Compound 5-4 (42 mg, 56.3043 mol, 92.6674% yield). MS: m/z: 746 [M+H]⁺.

A solution of Compound 5-4 (42 mg, 56.3043 μmol) and CsF (89 mg, 589.8987 μmol) in DMF (4 mL) was stirred for 6 hours at 35° C. under nitrogen atmosphere. The solution was diluted with water (30 mL) and extracted with EA (30 mL×2). The organic layer was dried over anhydrous Na₂SO₄ and concentrated under vacuum. The residue was purified by Prep-HPLC (C18 column, A: 0.1% TFA in water, B: CH₃CN, Gradient: 15% B to 35% B in 37 min at a flow rate of 60 mL/min, 240 nm). The mixture was adjusted to pH 8-9 with saturated NaHCO₃ and concentrated in vacuum. The aqueous layer was extracted with EA, the organic layer was washed with water twice, concentrated in vacuum to freeze-dried to give Compound 5 (16 mg). MS: m/z: 590 [M+H]⁺.

### Example 8

### 4-(4-(ethyl(trans-2-fluorocyclopropyl)amino)-8-fluoro-2-(((2R,7aS)-2-fluorotetrahydro-1H-pyrrolizin-7a(5H)-yl)methoxy)pyrido[4,3-d]pyrimidin-7-yl)-5-ethynyl-6-fluoronaphthalen-2-ol 2,2,2-trifluoroacetic acid (“Compound 6 (TFA salt)”)

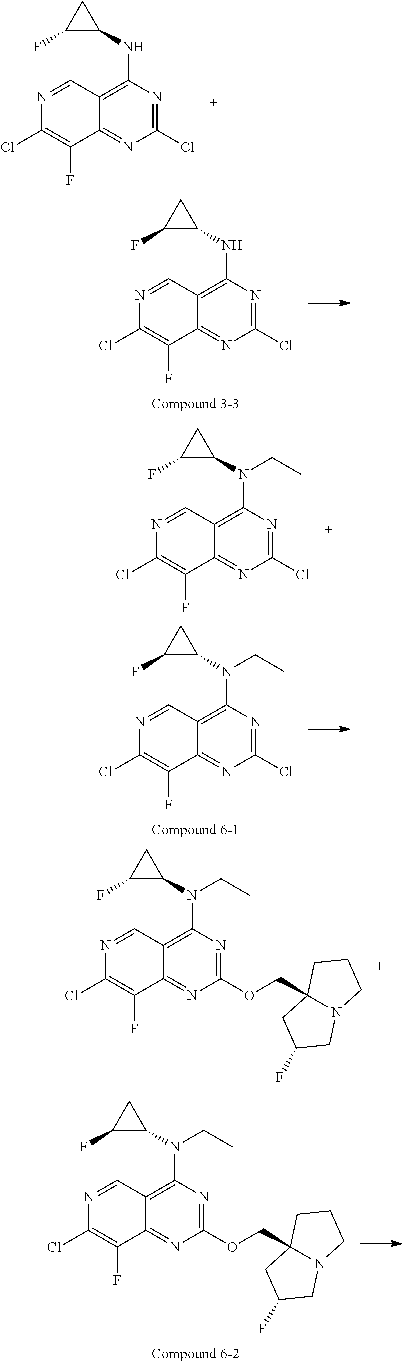

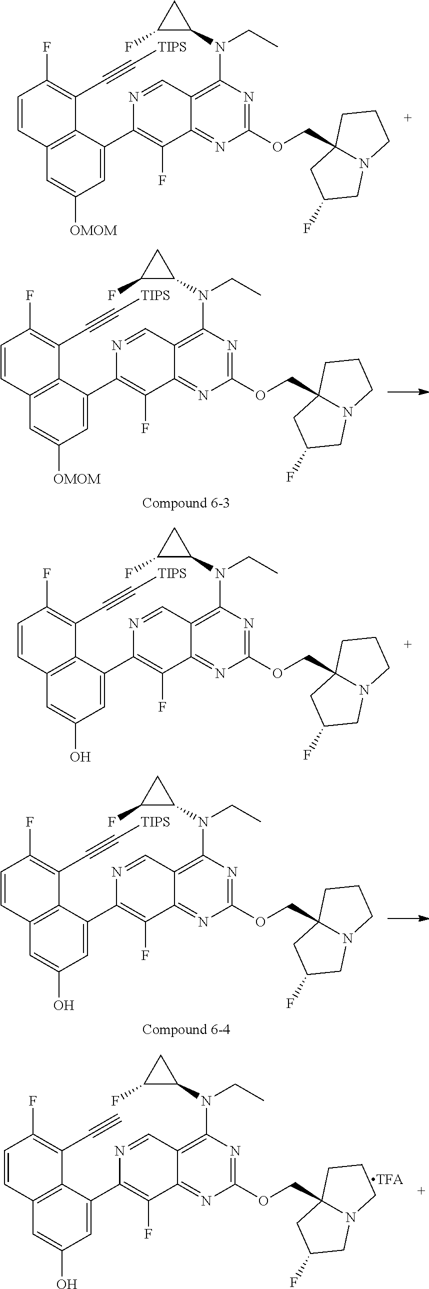

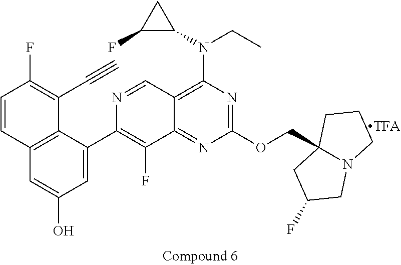 [SMILES: C#Cc1c(F)ccc2cc(O)cc(-c3ncc4c(N(CC)[C@H]5C[C@@H]5F)nc(OC[C@@]56CCCN5C[C@H](F)C6)nc4c3F)c12]

To a solution of Compound 3-3 (704 mg, 2.42 mmol) and iodoethane (1672 mg, 10.72 mmol) in DMF (10 mL) was added NaH (124 mg, 3.10 mmol, 60%). The mixture was stirred at room temperature for 24 h, then quenched with water (30 mL), extracted with EA (30 mL), washed with brine (20 mL×2), dried over anhydrous Na₂SO₄ and concentrated under reduced pressure. The residue was purified by Pre-TLC to give Compound 6-1 (45 mg, 0.14 mmol). MS m/z: 319/321[M+H]⁺.

A solution of Compound 6-1 (45 mg, 0.14 mmol), INT 2 (38 mg, 0.24 mmol) and KF (28 mg, 0.48 mmol) in DMSO (4 mL) was stirred at 95° C. for 19 h under nitrogen atmosphere. The mixture was allowed to cool to room temperature, diluted with water (30 mL) and extracted with EA (30 mL×2). The combined organic layers were washed with brine (40 mL), then dried over anhydrous Na₂SO₄ and concentrated under reduced pressure. The residue was purified by Pre-TLC to give Compound 6-2 (37 mg, 0.084 mmol). MS m/z: 442 [M+H]⁺.

To a solution of Compound 6-2 (37 mg, 0.084 mmol) and INT 3 (60 mg, 0.12 mmol) in toluene (4 mL) and water (1 mL) were added Cs₂CO₃ (61 mg, 0.19 mmol) and cataCXium A Pd G3 (25 mg, 0.034 mmol). The reaction mixture was stirred at 100° C. for 12 h under nitrogen atmosphere. The reaction was filtered and the filtrate was concentrated under reduced pressure. The residue was purified by Pre-TLC (DCM:MeOH=15:1, v/v) to give Compound 6-3 (50 mg, 0.063 mmol). MS m/z: 792 [M+H]⁺.

A solution of Compound 6-3 (50 mg, 0.063 mmol) and HCl (4 M in 1,4-dioxane, 1 mL) in ACN (3 mL) was stirred at room temperature for 1 h. The solution was diluted with saturated NaHCO₃ aqueous solution (20 mL) and extracted with EA (30 mL). The organic layer was washed with brine (30 mL), dried over anhydrous Na₂SO₄ and concentrated under reduced pressure to give Compound 6-4 (crude, 56 mg, 0.075 mmol). MS m/z: 748 [M+H]⁺.

A solution of Compound 6-4 (56 mg, 0.075 mmol) and CsF (0.34 g, 2.24 mmol) in DMF (5 mL) was stirred at 45° C. for 4 h. The mixture was diluted with water (30 mL) and extracted with EA (30 mL). The collected organic layer was dried over anhydrous Na₂SO₄ and concentrated under reduced pressure. The residue was purified by Prep-HPLC (C18 column, A: 0.1% TFA in water, B: CH₃CN, Gradient: 15% B to 44% B in 40 min at a flow rate of 40 mL/min, 235 nm) to give Compound 6 (TFA salt) (10.7 mg, 0.015 mmol). MS m/z: 592 [M+H]⁺.

### Example 9

### 5-ethynyl-6-fluoro-4-(8-fluoro-4-(((1S,2S)-2-fluorocyclopropyl)(methyl-d3)amino)-2-(((2R,7aS)-2-fluorotetrahydro-1H-pyrrolizin-7a(5H)-yl)methoxy)pyrido[4,3-d]pyrimidin-7-yl)naphthalen-2-ol 2,2,2-trifluoroacetic acid (“Compound 7 (TFA salt)”)

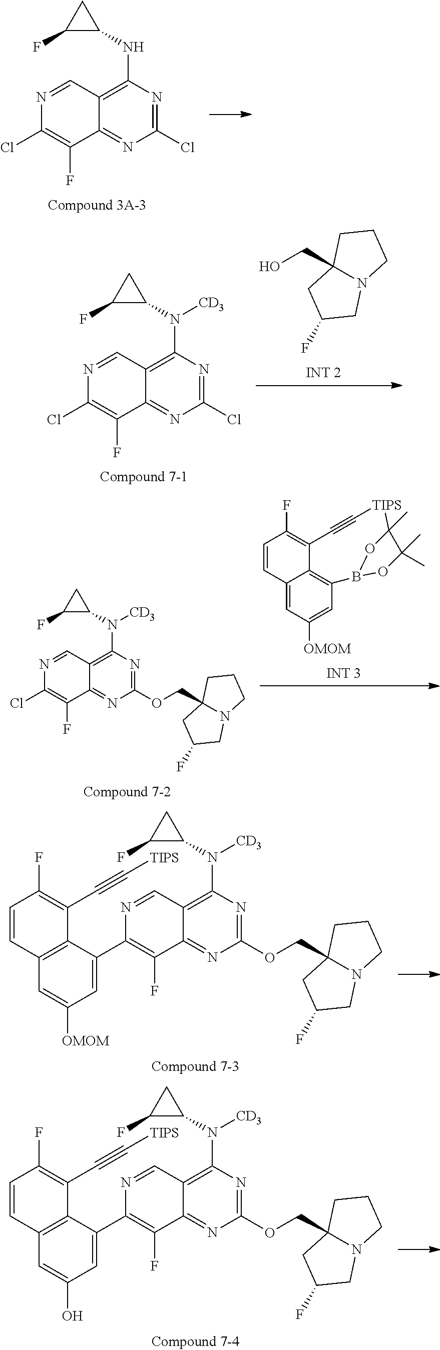

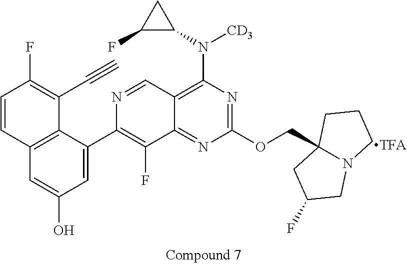 [SMILES: [2H]C([2H])([2H])N(c1nc(OC[C@@]23CCCN2C[C@H](F)C3)nc2c(F)c(-c3cc(O)cc4ccc(F)c(C#C)c34)ncc12)[C@H]1C[C@@H]1F]

A solution of Compound 3A-3 (478 mg, 1.64 mmol) and CD₃I (1024 mg, 7.06 mmol) in DMF (8 mL) was added NaH (90 mg, 2.25 mmol, 60%). Then the mixture was stirred at room temperature for 5 h. The solution was quenched with saturated NH₄Cl aqueous solution (2 mL), extracted with EA (30 mL), washed with brine (20 mL×2), dried over anhydrous Na₂SO₄ and concentrated in vacuum. The residue was purified by Pre-TLC (EA:Hex=1:3, v/v) to give Compound 7-1 (394 mg, 1.28 mmol, 77.87% yield). MS m/z: 308/310 [M+H]⁺.

A solution of Compound 7-1 (394 mg, 1.28 mmol) and INT 2 (207 mg, 1.30 mmol) in THF (7 mL) was added t-BuONa (149 mg, 1.55 mmol) portion wise at −10° C. under nitrogen atmosphere and then the mixture was stirred for 2 h, allowed to cool to room temperature and extracted with EA (30 mL). The organic layer was washed with brine (30 mL×2), then dried over anhydrous Na₂SO₄ and concentrated in vacuum. The residue was purified by Pre-TLC (DCM:MeOH=15:1, v/v) to give Compound 7-2 (358 mg, 0.831 mmol, 64.98% yield). MS m/z: 431 [M+H]⁺.

To a solution of Compound 7-2 (358 mg, 0.831 mmol) and INT 3 (523 mg, 1.02 mmol) in 1,4-dioxane (8 mL) and water (1 mL) was added Cs₂CO₃ (710 mg, 2.18 mmol) and cataCXium A Pd G3 (72 mg, 0.099 mmol). The reaction mixture was stirred at 100° C. overnight under nitrogen atmosphere. The reaction mixture was filtrated and the filtrate was concentrated under vacuum. The residue was purified by Pre-TLC (DCM:MeOH=15:1, v/v) to give Compound 7-3 as a brown solid: (484 mg, 0.620 mmol, 74.59% yield). MS m/z: 781 [M+H]⁺.

A solution of Compound 7-3 (484 mg, 0.620 mmol) and HCl (4 M in 1,4-dioxane, 1.5 mL) in ACN (6 mL) was stirred at room temperature for 2 h. The solution was diluted with saturated NaHCO₃ aqueous solution (20 mL) and extracted with EA (30 mL). The organic layer was washed with brine (30 mL), dried over anhydrous Na₂SO₄ and concentrated in vacuum to give crude Compound 7-4 (490 mg, 0.665 mmol, 107.29% yield). MS m/z: 737 [M+H]⁺.

A solution of Compound 7-4 (490 mg, 0.665 mmol) and CsF (0.82 g, 5.40 mmol) in DMF (6 mL) was stirred at 40° C. for 2 h under nitrogen atmosphere. The solution was diluted with water (10 mL) and extracted with EA (20 mL). The organic layer was dried over anhydrous Na₂SO₄ and concentrated under vacuum. The residue was purified by Prep-HPLC (C18 column, A: 0.1% TFA in water, B: CH₃CN, Gradient: 20% B to 46% B in 35 min at a flow rate of 70 mL/min, 240 nm) to freeze-dried to give Compound 7 (TFA salt) (220.5 mg, 0.380 mmol, 57.12% yield). MS m/z: 581 [M+H]⁺.

### Example 10

### 4-(4-(cyclopropyl(methyl-d3)amino)-8-fluoro-2-(((2R,7aS)-2-fluorotetrahydro-1H-pyrrolizin-7a(5H)-yl)methoxy)pyrido[4,3-d]pyrimidin-7-yl)-5-ethynyl-6-fluoronaphthalen-2-yl methylcarbamate (“Compound 8”)

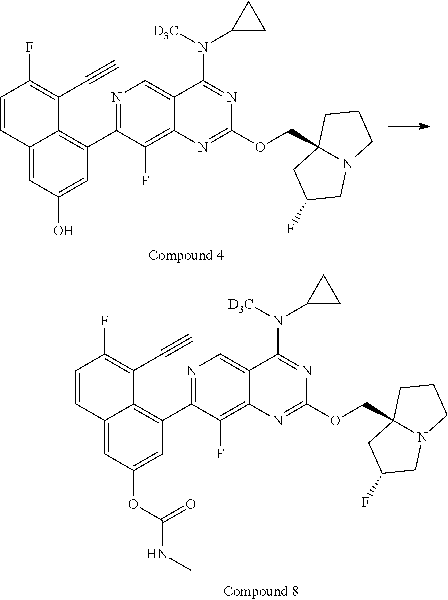 [SMILES: [2H]C([2H])([2H])N(c1nc(OC[C@@]23CCCN2C[C@H](F)C3)nc2c(F)c(-c3cc(O)cc4ccc(F)c(C#C)c34)ncc12)C1CC1 ; [2H]C([2H])([2H])N(c1nc(OC[C@@]23CCCN2C[C@H](F)C3)nc2c(F)c(-c3cc(OC(=O)NC)cc4ccc(F)c(C#C)c34)ncc12)C1CC1]

To a solution of Compound 4 (2.44 g, 4.34 mmol) and DIEA (8 ml) in DCM (150 mL) was added methylcarbamic chloride (1.20 g, 12.83 mmol). The reaction mixture was stirred at 30° C. for 4 hours. The reaction mixture was diluted with NH₄Cl aqueous solution (80 mL) and extracted with DCM (30 mL). The organic layer was dried over anhydrous Na₂SO₄ and concentrated under vacuum. The crude product was purified by Pre-HPLC-Gilson with the following conditions: Column, CHIRAL ART Cellulose SA column (2 cm×25 cm, 5 μm) mobile phase, Hex/EtOH (50:50); Flowing rate: 20 mL/min to give Compound 8 (1.668 g, 2.69 mmol). MS m/z: 620 [M+H]⁺.

### Example 11

### (2R,7aS)-7a-(((7-(8-ethynyl-7-fluoro-3-hydroxynaphthalen-1-yl)-8-fluoro-4-((trans-2-fluorocyclopropyl)(methyl)amino)pyrido[4,3-d]pyrimidin-2-yl)oxy)methyl)hexahydro-1H-pyrrolizin-2-ol 2,2,2-trifluoroacetic acid (“Compound 9 (TFA salt)”)

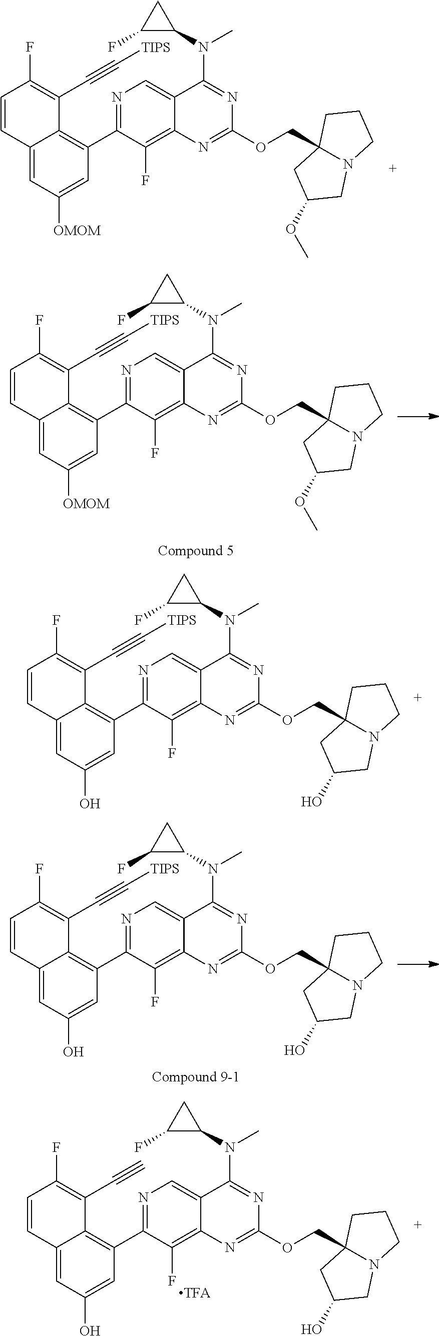

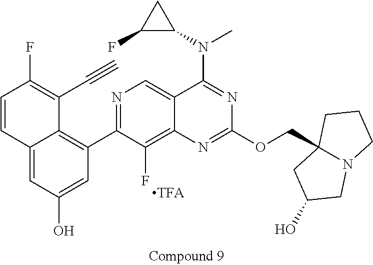 [SMILES: C#Cc1c(F)ccc2cc(O)cc(-c3ncc4c(N(C)[C@H]5C[C@@H]5F)nc(OC[C@@]56CCCN5C[C@H](O)C6)nc4c3F)c12]

A solution of Compound 5 (77 mg, 97.4685 μmol) in DCM (5 mL) was stirred at 5° C. under nitrogen atmosphere. A solution of BBr₃ (77 mg, 307.3570 μmol) in DCM (1 mL) was added to the mixture dropwise. The mixture was stirred for 48 h at room temperature, diluted with saturated NaHCO₃ (20 mL) aqueous solution and extracted with DCM (2×30 mL). The organic layer was washed with NaCl aqueous solution, then dried over anhydrous Na₂SO₄ and concentrated in vacuum. The residue was purified by Pre-TLC to give Compound 9-1 (19 mg, 24.4854 μmol, 25.1214% yield). MS: m/z: 732 [M+H]⁺.

A solution of Compound 9-1 (19 mg, 25.9591 μmol) and CsF (125 mg, 822.8914 μmol) in DMF (4 mL) was stirred for 3 hours at 35° C. under nitrogen atmosphere. The solution was diluted with water (30 mL) and extracted with EA (30 mL×2). The organic layer was dried over anhydrous Na₂SO₄ and concentrated under vacuum. The residue was purified by Prep-HPLC (C18 column, A: 0.1% TFA in water, B: CH₃CN, Gradient: 15% B to 40% B in 28 min at a flow rate of 40 mL/min, 234 nm) to freeze-dried to give Compound 9 (TFA salt) (1.8 mg). MS: m/z: 576 [M+H]⁺.

### Example 12

### 5-ethynyl-6-fluoro-4-(8-fluoro-2-(((2R,7aS)-2-fluorotetrahydro-1H-pyrrolizin-7a(5H)-yl)methoxy)-4-((1-methylcyclopropyl)amino)pyrido[4,3-d]pyrimidin-7-yl)naphthalen-2-ol (“Compound 10”)

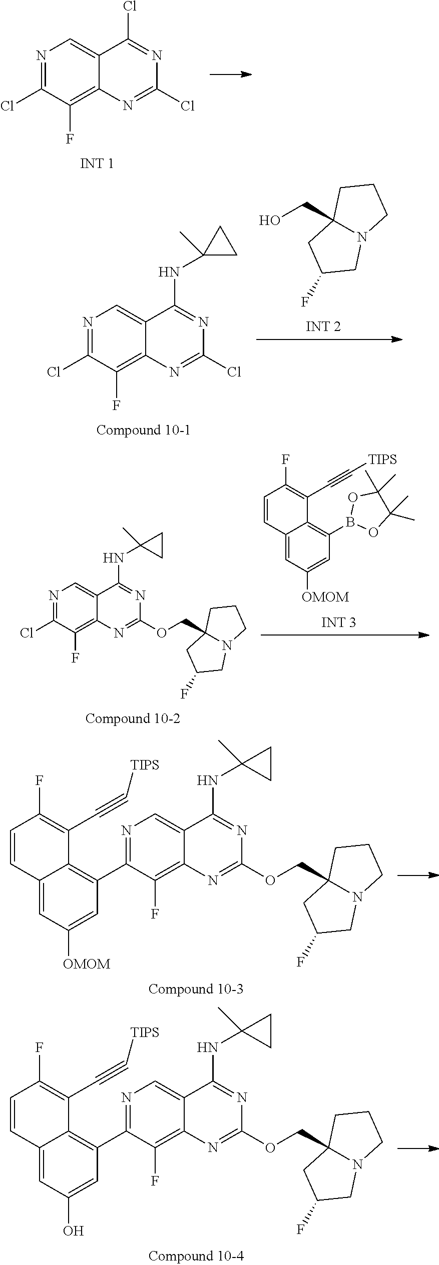

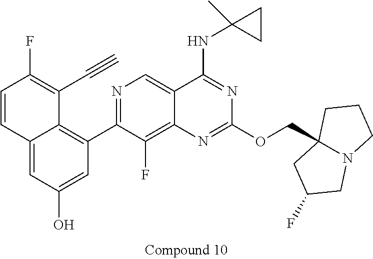 [SMILES: C#Cc1c(F)ccc2cc(O)cc(-c3ncc4c(NC5(C)CC5)nc(OC[C@@]56CCCN5C[C@H](F)C6)nc4c3F)c12]

A solution of INT 1 (280 mg, 1.1091 mmol), 1-methylcyclopropylamine hydrochloride (110 mg, 1.0225 mmol) and DIEA (474 mg, 3.6675 mmol) in DCM (5 mL) was stirred at room temperature for 2 h. The solution was diluted with 10% NaHCO₃ aqueous solution (10 mL) and extracted with DCM (20 mL×2). The organic layer was washed with NaCl aqueous solution, dried over anhydrous Na₂SO₄ and concentrated in vacuum to give Compound 10-1 (378 mg, crude). MS: m/z: 287 [M+H]⁺.

A solution of Compound 10-1 (378 mg, 1.3165 mmol), INT 2 (219 mg, 1.3756 mmol) and KF (364 mg, 6.2654 mmol) in DMSO (5 mL) was stirred at 90° C. for 16 h under nitrogen atmosphere. The mixture was allowed to cool to room temperature, diluted with saturated NaHCO₃ aqueous solution (20 mL) and extracted with EA (40 mL×2). The organic layer was washed with NaCl aqueous solution (20 mL), then dried over anhydrous Na₂SO₄ and concentrated in vacuum. The residue was purified by Pre-TLC to give Compound 10-2 (307 mg, 749.0366 μmol, 56.8951% yield). MS: m/z: 410 [M+H]⁺.

A solution of Compound 10-2 (146 mg, 356.2194 μmol), toluene (5 mL), INT-3 (241 mg, 470.2122 mol), cataCXium A Pd G3 (38 mg, 52.1785 μmol), cesium carbonate (348 mg, 1.0681 mmol) and water (1 mL) was stirred at 100° C. for 18 hours under nitrogen atmosphere. The mixture was allowed to cool to room temperature, diluted with saturated NaHCO₃ aqueous solution (10 mL) and extracted with EA (25 mL×2). The organic layer was washed with NaCl aqueous solution (10 mL), then dried over anhydrous Na₂SO₄ and concentrated in vacuum. The residue was purified by Pre-TLC to give Compound 10-3 (184 mg, 242.1140 μmol, 67.9677% yield). MS: m/z: 760 [M+H]⁺.

A solution of Compound 10-3 (184 mg, 242.1140 μmol) and HCl (4 M in 1,4-dioxane, 1 mL) in DCM (5 mL) was stirred at room temperature for 1 h. The solution was diluted with 10% NaHCO₃ aqueous solution (10 mL) and extracted with DCM (30 mL). The organic layer was washed with saturated NaCl aqueous solution, dried over anhydrous Na₂SO₄ and concentrated in vacuum to give crude Compound 10-4 (172 mg, 240.2503 μmol, 99.2303% yield). MS: m/z: 716 [M+H]⁺.

A solution of Compound 10-4 (172 mg, 240.2503 μmol) and CsF (179 mg, 1.1784 mmol) in DMF (5 mL) was stirred for 20 hours at 35° C. under nitrogen atmosphere. The solution was diluted with saturated NaHCO₃ aqueous solution (10 mL) and extracted with EA (10 mL). The organic layer was dried over anhydrous Na₂SO₄ and concentrated under vacuum. The residue was purified by Prep-HPLC (C18 column, A: 0.1% TFA in water, B: CH₃CN, Gradient: 15% B to 52% B in 37 min at a flow rate of 70 mL/min, 240 nm). The mixture was adjusted to pH=8 with saturated NaHCO₃ and concentrated in vacuum. The aqueous layer was extracted with EA, the organic layer was washed with water twice, concentrated in vacuum to freeze-dried to give Compound 10 (TFA salt) (54 mg). MS: m/z: 560 [M+H]⁺.

### Example 13

### 4-(4-((2,2-difluorocyclopropyl)(methyl)amino)-8-fluoro-2-(((2R,7aS)-2-fluorotetrahydro-1H-pyrrolizin-7a(5H)-yl)methoxy)pyrido[4,3-d]pyrimidin-7-yl)-5-ethynyl-6-fluoronaphthalen-2-ol 2,2,2-trifluoroacetic acid (“Compound 11 (TFA salt)”)

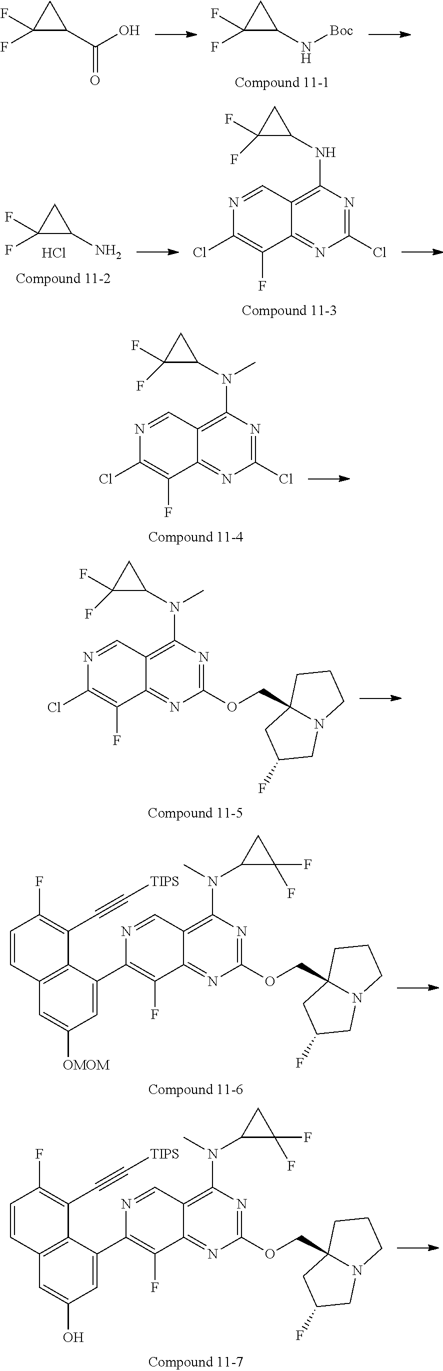

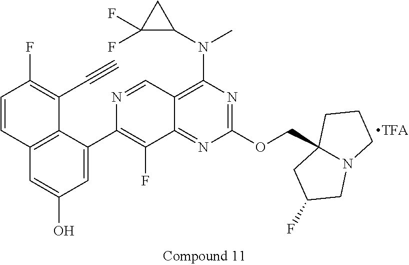 [SMILES: C#Cc1c(F)ccc2cc(O)cc(-c3ncc4c(N(C)C5CC5(F)F)nc(OC[C@@]56CCCN5C[C@H](F)C6)nc4c3F)c12]

A mixture of 2,2-difluorocyclopropane-1-carboxylic acid (4.95 g, 40.55 mmol), DPPA (13.73 g, 49.89 mmol) and TEA (4.82 g, 47.70 mmol) in t-BuOH (30 mL) was stirred at 90° C. for 16.5 h. The reaction mixture was concentrated under reduced pressure and diluted with water (40 mL) and EA (40 mL). The organic layer was washed with brine (50 mL), dried over anhydrous Na₂SO₄ and concentrated under reduced pressure. The residue was purified by column chromatography (Hex:EA=10:1, v/v) to give Compound 11-1 (7.34 g, 93.69%).

A solution of Compound 11-1 (1.97 g, 10.20 mmol) and HCl (4 M in 1,4-dioxane, 5 mL) in DCM (5 mL) was stirred at room temperature for 2 h. The solution was concentrated under reduced pressure to give Compound 11-2 (crude). Compound 11-2 (crude) was added to a solution of 2,4,7-trichloro-8-fluoropyrido[4,3-d]pyrimidine (1.36 g, 5.39 mmol) and DIEA (2.70 g, 20.89 mmol) in DCM (10 mL). The mixture was stirred at room temperature for 17 h, then diluted with water (10 mL). The organic layer was concentrated under reduced pressure. The residue was purified by column chromatography (Hex:EA=2:1, v/v) to give Compound 11-3 (1.18 g, 70.87%). MS m/z: 309/311 [M+H]⁺.

To a solution of Compound 11-3 (0.59 g, 1.91 mmol) and CH₃I (0.61 g, 4.30 mmol) in DMF (5 mL) was added NaH (0.11 g, 2.75 mmol, 60% content). The mixture was stirred at room temperature for 17 h, quenched with water (20 mL), extracted with EA (30 mL×2) and concentrated under reduced pressure. The residue was purified by Pre-TLC (EA:Hex=1:1, v/v) to give Compound 11-4 (0.13 g, 21.07%). MS m/z: 323/325 [M+H]⁺.

A mixture of Compound 11-4 (0.13 g, 0.40 mmol), INT 2 (156 mg, 0.98 mmol) and KF (125 mg, 2.15 mmol) in DMSO (3 mL) was stirred at 90° C. for 5 h under nitrogen atmosphere. The mixture was allowed to cool to room temperature and diluted with water (20 mL) and EA (20 mL). The collected organic layer was washed with brine (20 mL) and concentrated under reduced pressure. The residue was purified by Pre-TLC (DCM:MeOH=11:1, v/v) to give Compound 11-5 (153 mg, 85.29%). MS m/z: 446 [M+H]⁺.

To a solution of Compound 11-5 (153 mg, 0.34 mmol) and INT 3 (263 mg, 0.51 mmol) in toluene (5 mL) and water (1 mL) were added Cs₂CO₃ (228 mg, 0.70 mmol) and cataCXium A Pd G3 (40 mg, 0.05 mmol). The reaction mixture was stirred at 100° C. overnight under nitrogen atmosphere. The mixture was allowed to cool to room temperature and diluted with water (10 mL) and EA (10 mL). The organic layer was concentrated under reduced pressure. The residue was purified by Pre-TLC (DCM:MeOH=10:1, v/v) to give Compound 11-6 (174 mg, 63.70%). MS m/z: 796 [M+H]⁺.

A solution of Compound 11-6 (174 mg, 0.22 mmol) and HCl (4 M in 1,4-dioxane, 2 mL) in DCM (10 mL) was stirred at room temperature for 1 h. The mixture was diluted with water (10 mL) and EA (10 mL) and the pH was adjusted to 9 with K₂CO₃. The collected organic layer was concentrated under reduced pressure to give Compound 11-7 (crude). MS m/z: 752 [M+H]⁺.

A mixture of Compound 11-7 (crude) and CsF (0.57 g, 3.75 mmol) in DMF (5 mL) was stirred at room temperature for 18 h under nitrogen atmosphere. The mixture was filtered and the filtrate was concentrated under reduced pressure. The residue was purified by Prep-HPLC (C18 column, A: 0.1% TFA in water, B: CH₃CN, Gradient: 15% B to 50% B in 46 min at a flow rate of 60 mL/min, 230 nm) to give freeze-dried Compound 11 (TFA salt) (44.8 mg). MS m/z: 596 [M+H]⁺.

### Example 14

### 5-ethynyl-6-fluoro-4-(8-fluoro-4-(((1S,2R)-2-fluorocyclopropyl)(methyl)amino)-2-(((2R,7aS)-2-fluorotetrahydro-1H-pyrrolizin-7a(5H)-yl)methoxy)pyrido[4,3-d]pyrimidin-7-yl)naphthalen-2-ol 2,2,2-trifluoroacetic acid (“Compound 12 (TFA salt)”)

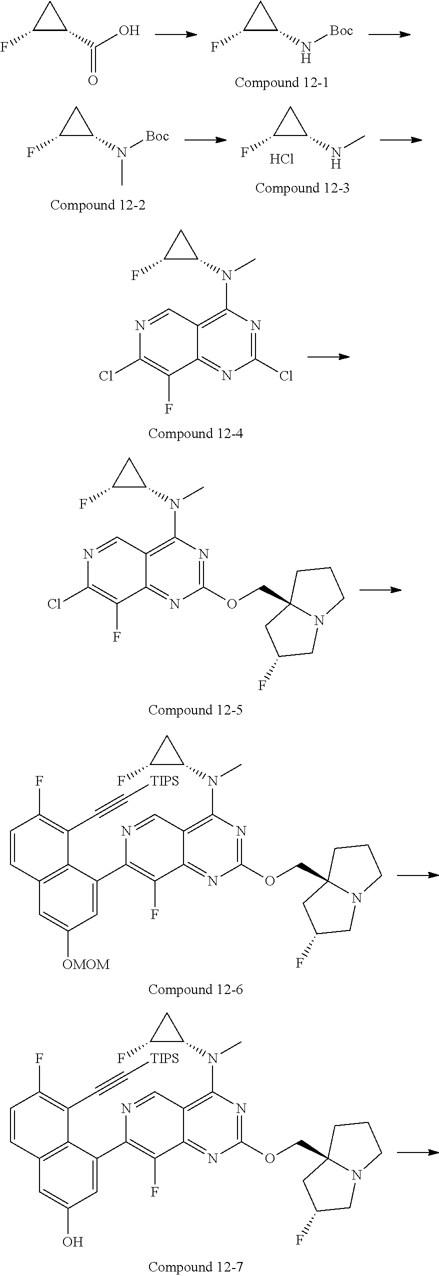

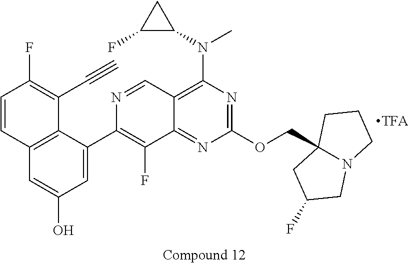 [SMILES: C#Cc1c(F)ccc2cc(O)cc(-c3ncc4c(N(C)[C@H]5C[C@H]5F)nc(OC[C@@]56CCCN5C[C@H](F)C6)nc4c3F)c12]

A mixture of (1R,2R)-2-fluorocyclopropane-1-carboxylic acid (4.16 g, 39.97 mmol), DPPA (12.21 g, 47.97 mmol) and TEA (4.91 g, 48.52 mmol) in t-BuOH (45 mL) was stirred at 90° C. for 16 h. The reaction mixture was concentrated under reduced pressure and diluted with water (40 mL) and EA (40 mL). The organic layer was washed with brine (50 mL), dried over anhydrous Na₂SO₄ and concentrated under reduced pressure. The residue was purified by column chromatography (Hex:EA=10:1, v/v) to give Compound 12-1 (7.04 g, 100.53%).

To a solution of Compound 12-1 (1.31 g, 7.48 mmol) and CH₃I (1.3 g, 9.16 mmol) in DMF (10 mL) was added NaH (0.35 g, 8.75 mmol, 60% content). The mixture was stirred at room temperature for 25 h, quenched with water (30 mL), extracted with EA (30 mL), washed with brine (30 mL) and concentrated under reduced pressure. The residue was purified by column chromatography (EA:Hex=1:10, v/v) to give Compound 12-2 (0.66 g, 46.65%). MS m/z: 134 [M+H−56]⁺.

A solution of Compound 12-2 (0.66 g, 3.49 mmol) and HCl (4 M in dioxane, 2 mL) in DCM (10 mL) was stirred at room temperature for 17.5 h. The solution was concentrated under reduced pressure to give Compound 12-3 (crude). Compound 12-3 (crude) was added to a solution of 2,4,7-trichloro-8-fluoropyrido[4,3-d]pyrimidine (600 mg, 2.38 mmol) and DIEA (1 g, 7.74 mmol) in DCM (10 mL). The mixture was stirred at room temperature for 2 h and diluted with water (10 mL). The organic layer was concentrated under reduced pressure. The residue was purified by Pre-TLC (Hex:EA=1:1, v/v) to give Compound 12-4 (0.18 g, 16.96%). MS m/z: 305/307 [M+H]⁺.

A mixture of Compound 12-4 (0.18 g, 0.59 mmol), INT 2 (234 mg, 1.47 mmol) and KF (187 mg, 3.22 mmol) in DMSU (5 mL) was stirred at 90° C. for 3.5 h under nitrogen atmosphere. The mixture was allowed to cool to room temperature and diluted with water (10 mL) and EA (10 mL). The organic layer was concentrated under reduced pressure. The residue was purified by Pre-TLC (DCM:MeOH=10:1, v/v) to give Compound 12-5 (212 mg, 83.99%). MS m/z: 428 [M+H]⁺.

To a solution of Compound 12-5 (212 mg, 0.50 mmol), INT 3 (337 mg, 0.66 mmol) in toluene (5 mL) and water (1 mL) was added Cs₂CO₃ (407 mg, 1.25 mmol) and cataCXium A Pd G3 (36 mg, 0.05 mmol). The reaction mixture was stirred at 100° C. overnight under nitrogen atmosphere. The mixture was allowed to cool to room temperature and diluted with water (10 mL) and EA (10 mL). The organic layer was collected and concentrated under reduced pressure. The residue was purified by Pre-TLC (DCM:MeOH=12:1, v/v) to give Compound 12-6 (227 mg, 58.89%). MS m/z: 778 [M+H]⁺.

A solution of Compound 12-6 (227 mg, 0.29 mmol) and HCl (4 M in 1,4-dioxane, 2 mL) in DCM (10 mL) was stirred at room temperature for 2 h. The mixture was diluted with water (10 mL) and EA (10 mL) and the pH was adjusted to 9 with K₂CO₃. The organic layer was collected and concentrated under reduced pressure to give Compound 12-7 (crude). MS m/z: 734 [M+H]⁺.

A mixture of Compound 12-7 (crude) and CsF (0.82 g, 5.40 mmol) in DMF (5 mL) was stirred at room temperature for 1.5 h under nitrogen atmosphere. The mixture was filtered and the filtrate was concentrated under reduced pressure. The residue was purified by Prep-HPLC (C18 column, A: 0.1% TFA in water, B: CH₃CN, Gradient: 15% B to 32% B in 32 min at a flow rate of 60 mL/min, 230 nm) to give freeze-dried Compound 12 (TFA salt) (89.6 mg). MS m/z: 578 [M+H]⁺.

### Example 15

5-ethynyl-6-fluoro-4-(8-fluoro-4-(((1R,2S)-2-fluorocyclopropyl)(methyl)amino)-2-(((2R,7aS)-2-fluorotetrahydro-1H-pyrrolizin-7a(5H)-yl)methoxy)pyrido[4,3-d]pyrimidin-7-yl)naphthalen-2-ol

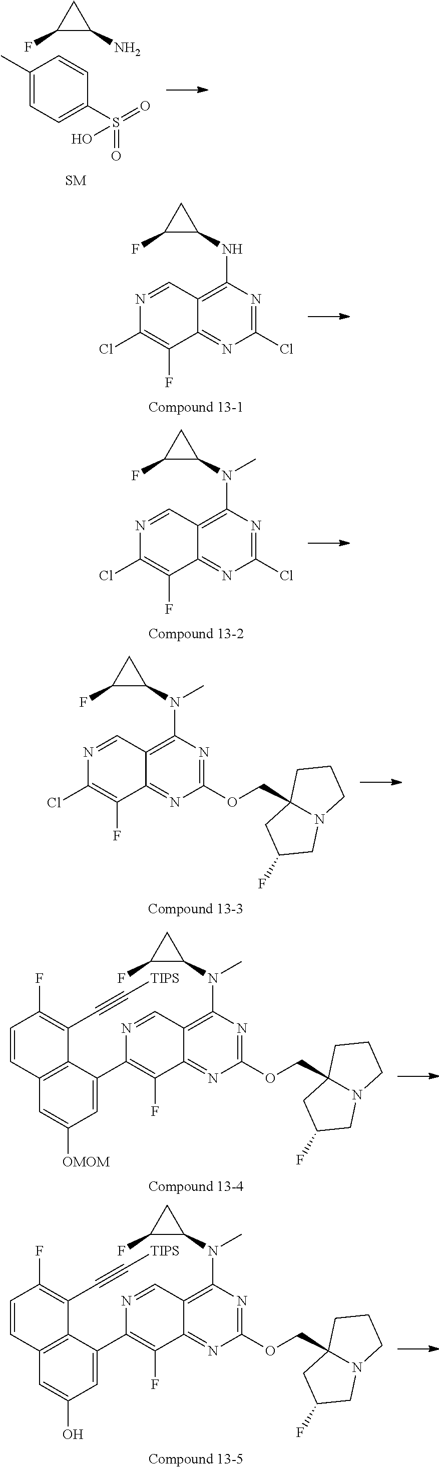

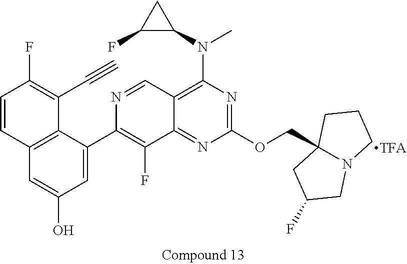 [SMILES: C#Cc1c(F)ccc2cc(O)cc(-c3ncc4c(N(C)[C@@H]5C[C@@H]5F)nc(OC[C@@]56CCCN5C[C@H](F)C6)nc4c3F)c12]

SM (488 mg, 1.97 mmol) was added to a solution of 2,4,7-trichloro-8-fluoropyrido[4,3-d]pyrimidine (491 mg, 1.94 mmol) and DIEA (0.72 g, 5.57 mmol) in DCM (10 mL). The mixture was stirred at room temperature for 0.2 h and diluted with DCM (30 mL). The reaction mixture was washed with water (50 mL) and concentrated under reduced pressure. The residue was added to a solution of EA (3 mL) and Hexane (10 mL), stirred for 1.5 h and filtered to give Compound 13-1 (0.52 g, 91.85%). MS m/z: 291/293 [M+H]⁺.

To a solution of Compound 13-1 (491 mg, 1.69 mmol) and CH₃I (965 mg, 6.80 mmol) in DMF (8 mL) was added NaH (132 mg, 5.5 mmol, 60% content). The mixture was stirred at room temperature for 3.5 h, quenched with water (20 mL), extracted with EA (20 mL×2) and concentrated under reduced pressure to give Compound 13-2 (0.54 g, 104.92%). MS m/z: 305/307 [M+H]⁺.

A mixture of Compound 13-2 (0.27 g, 0.88 mmol), INT 2 (268 mg, 1.68 mmol) and KF (304 mg, 5.23 mmol) in DMSO (3 mL) were stirred at 90° C. for 3.5 h under nitrogen atmosphere. The mixture was allowed to cool to room temperature and diluted with water (20 mL) and EA (20 mL). The organic layer was washed with brine (20 mL) and concentrated under reduced pressure. The residue was purified by Pre-TLC (EA) to give Compound 13-3 (91 mg, 24.03%). MS m/z: 428 [M+H]⁺.

To a solution of Compound 13-3 (91 mg, 0.21 mmol) and INT 3 (159 mg, 0.31 mmol) in toluene (5 mL) and water (1 mL) were added Cs₂CO₃ (225 mg, 0.69 mmol) and cataCXium A Pd G3 (44 mg, 0.06 mmol). The reaction mixture was stirred at 100° C. overnight under nitrogen atmosphere. The mixture was allowed to cool to room temperature, diluted with water (10 mL) and EA (10 mL). The organic layer was concentrated under reduced pressure. The residue was purified by Pre-TLC (DCM:MeOH=10:1, v/v) to give Compound 13-4 (70 mg, 42.30%). MS m/z: 778 [M+H]⁺.

A solution of Compound 13-4 (70 mg, 0.09 mmol) and HCl (4 M in dioxane, 2 mL) in DCM (5 mL) was stirred at room temperature for 2 h. The mixture was diluted with water (10 mL) and EA (10 mL) and the pH was adjusted to 9 with K₂CO₃. The collected organic layer was concentrated under reduced pressure to give Compound 13-5 (crude). MS m/z: 734 [M+H]⁺.

A solution of Compound 13-5 (crude) and CsF (0.28 g, 1.84 mmol) in DMF (2 mL) was stirred at room temperature for 3 h under nitrogen atmosphere. The mixture was filtered and the filtrate was concentrated under reduced pressure. The residue was purified by Prep-HPLC (C18 column, A: 0.1% TFA in water, B: CH₃CN, Gradient: 15% B to 36% B in 35 min at a flow rate of 60 mL/min, 230 nm) to give freeze-dried Compound 13 (TFA salt) (28.8 mg). MS m/z: 578 [M+H]⁺.

### Example 16

### 4-(4-(cyclopropyl(cyclopropylmethyl)amino)-8-fluoro-2-(((2R,7aS)-2-fluorotetrahydro-1H-pyrrolizin-7a(5H)-yl)methoxy)pyrido[4,3-d]pyrimidin-7-yl)-5-ethynyl-6-fluoronaphthalen-2-ol

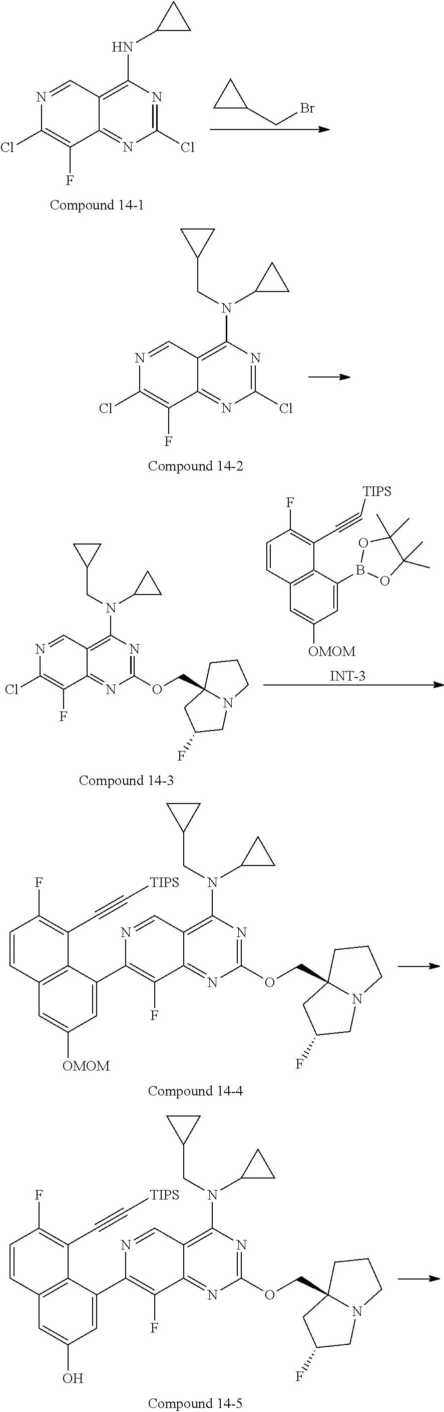

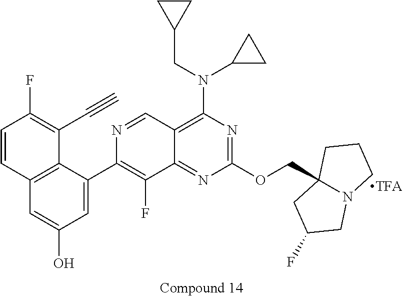 [SMILES: C#Cc1c(F)ccc2cc(O)cc(-c3ncc4c(N(CC5CC5)C5CC5)nc(OC[C@@]56CCCN5C[C@H](F)C6)nc4c3F)c12]

To a solution of Compound 14-1 (407 mg, 1.49 mmol) and (bromomethyl)cyclopropane (618 mg, 4.58 mmol) in ACN (8 mL) was added Cs₂CO₃ (1.07 g, 3.30 mmol). The mixture was stirred at 80° C. overnight, quenched with saturated NH₄Cl aqueous solution (2 mL) and extracted with EA (30 mL). The organic layer was washed with brine (20 mL×2), dried over anhydrous Na₂SO₄ and concentrated under reduced pressure. The residue was purified by Pre-TLC (EA:Hex=1:3, v/v) to give Compound 14-2 (133 mg, 0.406 mmol, 27.28% yield). MS m/z: 327/329 [M+H]⁺.

To a solution of Compound 14-2 (133 mg, 0.406 mmol) and INT 2 (102 mg, 0.641 mmol) in DMSO (5 mL) was added KF (71 mg, 1.22 mmol). The reaction was stirred at 98° C. overnight under nitrogen atmosphere. The mixture was allowed to cool to room temperature and extracted with EA (30 mL). The organic layer was washed with brine (30 mL×2), dried over anhydrous Na₂SO₄ and concentrated under reduced pressure. The residue was purified by Pre-TLC (DCM:MeOH=20:1, v/v) to give Compound 14-3 (123 mg, 0.273 mmol, 67.25% yield). MS m/z: 450 [M+H]⁺.

To a solution of Compound 14-3 (123 mg, 0.273 mmol) and INT 3 (175 mg, 0.341 mmol) in 1,4-dioxane (8 mL) and water (1 mL) was added Cs₂CO₃ (243 mg, 0.746 mmol) and cataCXium A Pd G3 (27 mg, 0.037 mmol). The reaction mixture was stirred at 100° C. overnight under nitrogen atmosphere. The reaction mixture was filtered and the filtrate was concentrated under reduced pressure. The residue was purified by Pre-TLC (DCM:MeOH=20:1, v/v) to give Compound 14-4 as a brown solid (118 mg, 0.147 mmol, 53.95% yield). MS m/z: 800 [M+H]⁺.

To a solution of Compound 14-4 (78 mg, 0.097 mmol) and HCl (4 M in 1,4-dioxane, 1 mL) in ACN (4 mL) was stirred at room temperature for 2 h. The solution was diluted with saturated NaHCO₃ aqueous solution (20 mL) and extracted with EA (30 mL). The organic layer was washed with brine (30 mL), dried over anhydrous Na₂SO₄ and concentrated under reduced pressure to give crude Compound 14-5 (65 mg, 0.086 mmol, 88.19% yield). MS m/z: 756 [M+H]⁺.

A mixture of Compound 14-5 (65 mg, 0.086 mmol) and CsF (155 mg, 1.02 mmol) in DMF (3 mL) was stirred at 40° C. for 2 h under nitrogen atmosphere. The mixture was diluted with water (10 mL) and extracted with EA (20 mL). The organic layer was dried over anhydrous Na₂SO₄ and concentrated under reduced pressure. The residue was purified by Prep-HPLC (C18 column, A: 0.1% TFA in water, B: CH₃CN, Gradient: 20% B to 46% B in 35 min at a flow rate of 70 mL/min, 240 nm) to give freeze-dried Compound 14 (TFA salt) (34.5 mg, 0.048 mmol, 56.22% yield). MS m/z: 600 [M+H]⁺.

### Example 17

### 5-ethyl-6-fluoro-4-(8-fluoro-4-((trans-2-fluorocyclopropyl)(methyl)amino)-2-(((2R,7aS)-2-fluorotetrahydro-1H-pyrrolizin-7a(5H)-yl)methoxy)pyrido[4,3-d]pyrimidin-7-yl)naphthalen-2-ol 2,2,2-trifluoroacetic acid (“Compound 15 (TFA salt)”)

### 6-fluoro-4-(8-fluoro-4-((trans-2-fluorocyclopropyl)(methyl)amino)-2-(((2R,7aS)-2-fluorotetrahydro-1H-pyrrolizin-7a(5H)-yl)methoxy)pyrido[4,3-d]pyrimidin-7-yl)-5-vinylnaphthalen-2-ol 2,2,2-trifluoroacetic acid (“Compound 16 (TFA salt)”)

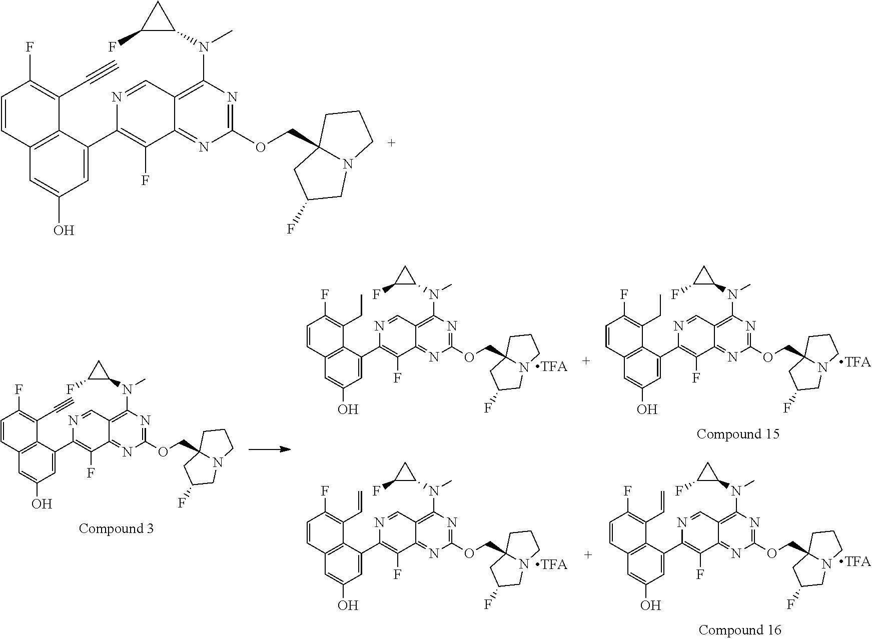

To a solution of Compound 3 (208 mg, 0.360 mmol) in MeOH (8 mL) was added Pd/C (84 mg, 0.079 mmol, 10% wt) under hydrogen atmosphere. The reaction was stirred for 4 h at room temperature. Then, the mixture was filtered and the filtrate was concentrated under reduced pressure. The residue was purified by Prep-HPLC (C18 column, A: 0.1% TFA in water, B: CH₃CN, Gradient: 15% B to 46% B in 45 min at a flow rate of 60 mL/min, 230 nm) to give freeze-dried Compound 15 (TFA salt) (148.9 mg, 0.214 mmol, 59.44% yield), MS m/z: 582 [M+H]+ and Compound 16 (TFA salt) (31.7 mg, 0.046 mmol, 12.69% yield), MS m/z: 580 [M+H]⁺.

### Example 18

### 4-(4-(cyclopropyl(2,2-difluoroethyl)amino)-8-fluoro-2-(((2R,7aS)-2-fluorotetrahydro-1H-pyrrolizin-7a(5H)-yl)methoxy)pyrido[4,3-d]pyrimidin-7-yl)-5-ethynyl-6-fluoronaphthalen-2-ol 2,2,2-trifluoroacetic acid (“Compound 17 (TFA salt)”)

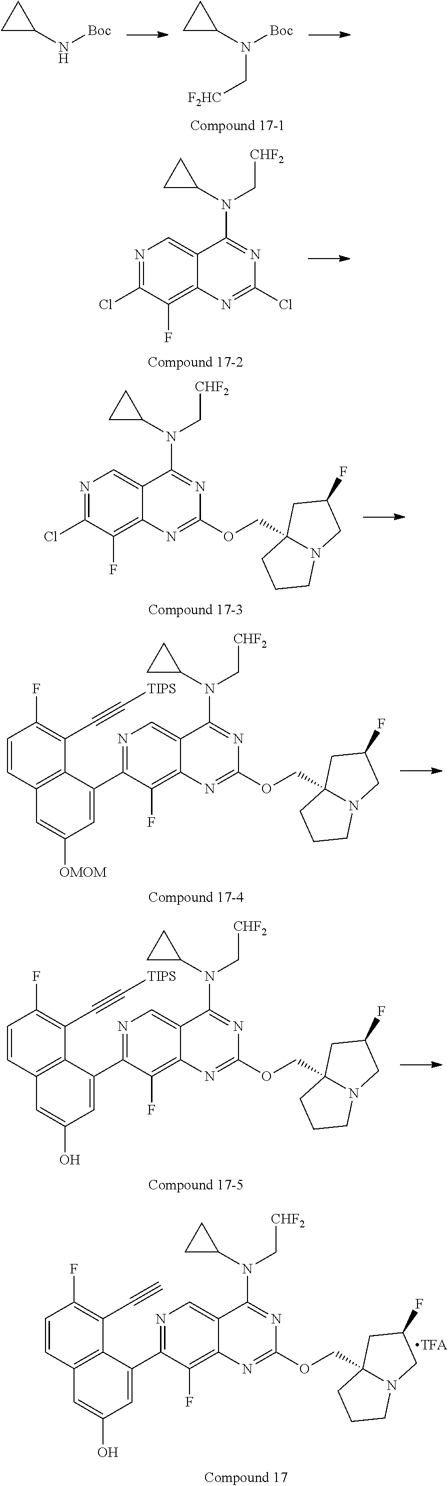

To a solution of tert-butyl cyclopropylcarbamate (2.70 g, 17.17 mmol) in DMF (35 mL) was added NaH (1.26 g, 31.50 mmol, 60% content) at −10° C. After stirring for 0.5 h, 1,1-difluoro-2-iodoethane (4.5 g, 23.44 mmol) was added to the reaction mixture. The reaction was stirred at room temperature for 16 h, quenched with water (80 mL), extracted with EA (50 mL), washed with brine (50 mL×2), dried over anhydrous Na₂SO₄ and concentrated under reduced pressure. The residue was purified by silica gel column to give Compound 17-1 (360 mg, 1.63 mmol). MS m/z: 222 [M+H]⁺.

To a solution of Compound 17-1 (360 mg, 1.63 mmol) in ACN (9 mL) was added HCl (3 mL, 4 M in dioxane). The reaction mixture was stirred at room temperature for 1.5 h and concentrated under reduced pressure to give an off white solid. The solid was added to a solution of 2,4,7-trichloro-8-fluoropyrido[4,3-d]pyrimidine (479 mg, 1.90 mmol) and DIEA (935 mg, 7.23 mmol) in ACN (10 mL). The mixture was stirred at 50° C. for 2 h, diluted with water (30 mL) and extracted with EA (30 mL×2). The organic layer was washed with brine (20 mL×2), dried over anhydrous Na₂SO₄ and concentrated under reduced pressure. The residue was purified by Pre-TLC to give Compound 17-2 (179 mg, 0.53 mmol). MS m/z: 337 [M+H]⁺.

A mixture of Compound 17-2 (179 mg, 0.53 mmol), INT 2 (135 mg, 0.85 mmol) and KF (100 mg, 1.72 mmol) in DMSO (10 mL) was stirred at 95° C. for 17 h under nitrogen atmosphere. The mixture was allowed to cool to room temperature, diluted with water (30 mL) and extracted with EA (2×30 mL). The organic layer was washed with NaCl aqueous solution (30 mL), dried over anhydrous Na₂SO₄ and concentrated under reduced pressure. The residue was purified by Pre-TLC to give Compound 17-3 (128 mg, 278.34 μmol). MS: m/z: 460 [M+H]⁺.

To a solution of Compound 17-3 (121 mg, 0.26 mmol) and INT 3 (179 mg, 0.35 mmol) in toluene (6 mL) and water (1.5 mL) were added Cs₂CO₃ (177 mg, 0.54 mmol) and cataCXium A Pd G3 (39 mg, 0.054 mmol).

The reaction mixture was stirred at 100° C. overnight under nitrogen atmosphere. The mixture was allowed to cool to room temperature, diluted with water (30 mL) and extracted with EA (2×30 mL). The organic layers were combined, washed with NaCl aqueous solution (30 mL), dried over anhydrous Na₂SO₄ and concentrated under reduced pressure. The residue was purified by Pre-TLC to give Compound 17-4 (140 mg, 0.17 mmol). MS m/z: 810 [M+H]⁺.

A solution of Compound 17-4 (140 mg, 172.84 μmol) and HCl (4 M in 1,4-dioxane, 1.5 mL) in ACN (4.5 mL) was stirred at room temperature for 1 h. The solution was concentrated under reduced pressure, diluted with saturated NaHCO₃ aqueous solution (20 mL) and extracted with EA (30 mL×2). The organic layer was washed with brine (30 mL), dried over anhydrous Na₂SO₄ and concentrated under reduced pressure to give Compound 17-5 (crude, 136 mg, 177.56 μmol). MS m/z: 766 [M+H]⁺.

A mixture of Compound 17-5 (136 mg, 177.56 μmol) and CsF (0.37 g, 2.44 mmol) in DMF (5 mL) was stirred at 40° C. for 2 h. The mixture was diluted with water (30 mL) and extracted with EA (30 mL). The organic layer was dried over anhydrous Na₂SO₄ and concentrated under reduced pressure. The residue was purified by Prep-HPLC (C18 column, A: 0.1% TFA in water, B: CH₃CN, Gradient: 15% B to 43% B in 30 min at a flow rate of 60 mL/min, 230 nm) to give freeze-dried Compound 17 (TFA salt) (66.9 mg, 92.45 μmol). MS m/z: 610 [M+H]⁺.

### Example 19

### 4-(4-(cyclopropylamino)-8-fluoro-2-(((2R,7aS)-2-fluorotetrahydro-1H-pyrrolizin-7a(5H)-yl)methoxy)pyrido[4,3-d]pyrimidin-7-yl)-5-ethynyl-6-fluoronaphthalen-2-ol formic acid (“Compound 18 (HCOOH salt)”)

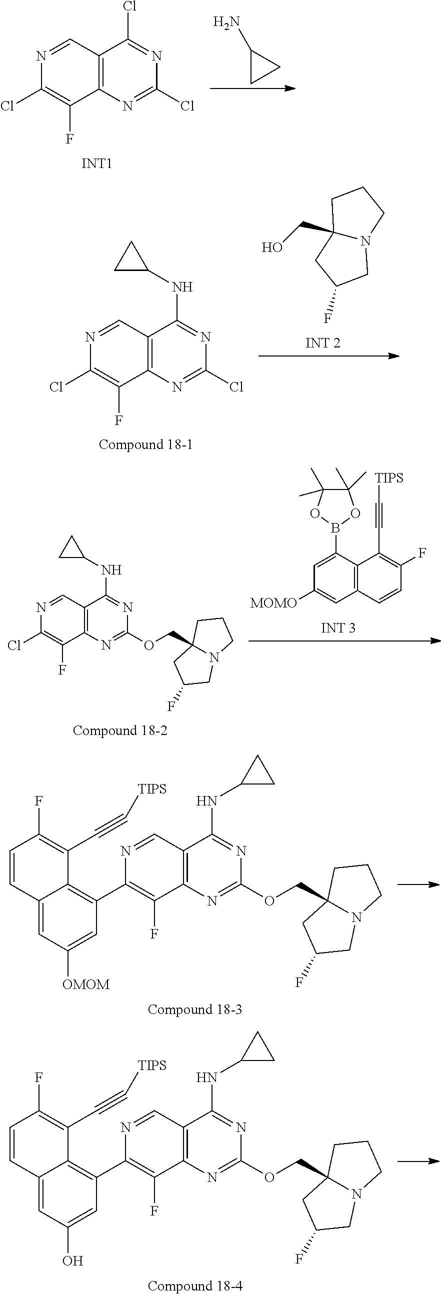

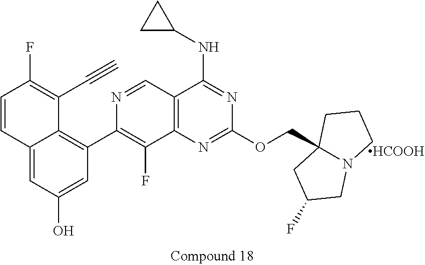

To a solution of INT 1 (200 mg, 0.80 mmol) in DCM (4 mL) was added DIEA (692 mg, 5.34 mmol) at room temperature. The mixture was cooled to −40° C. under argon atmosphere and cyclopropanamine (46 mg, 0.80 mmol) in DCM (0.5 mL) was added dropwise. After that the reaction mixture was stirred for 1 h at −40° C.

Then the reaction mixture was quenched with water (4 mL) and extracted with DCM (8 mL). The organic layer was washed with brine (5 mL), dried over anhydrous Na₂SO₄, filtered and the filtrate was concentrated to dryness to give Compound 18-1 (220 mg, crude) as a yellow solid. MS m/z: 273[M+H]⁺.

To a solution of Compound 18-1 (270 mg crude, 0.99 mmol) and INT 2 (315 mg, 1.98 mmol) in 1,4-dioxane (5 mL) was added DIEA (385 mg, 2.91 mmol) and 4A MS at room temperature. Then the reaction mixture was heated to 80° C. and stirred overnight. After that, the mixture was cooled to room temperature, poured into water (5 mL) and extracted with EtOAc (5 mL×3). The organic layer was washed with water (5 mL), brine (5 mL), dried over anhydrous Na₂SO₄, filtered and the filtrate was concentrated to dryness. The residue was purified by Pre-TLC (eluted with Petroleum ether/EtOAc=1:1) to give Compound 18-2 (200 mg, yield 63.8% for two steps) as a yellow solid. MS m/z: 396 [M+H]⁺.

Compound 18-2 (150 mg, 0.38 mmol), INT 3 (300 mg, 0.57 mmol), Cs₂CO₃ (369 mg, 1.14 mmol) and Pd(dppf)Cl₂ (84 mg, 0.11 mmol) were added successively into toluene/water=3:1 (3 mL), degassing the reaction with argon for 10 min. The mixture was heated to 100° C. under argon atmosphere and stirred for 12 h. After that, the mixture was cooled to room temperature, poured into water (5 mL) and extracted with EtOAc (5 mL×3). The combined organic layers were washed with water (5 mL), brine (5 mL), dried with anhydrous Na₂SO₄, filtered and the filtrate was concentrated to dryness. The residue was purified by Pre-TLC (eluted with DCM/MeOH=10:1) to give Compound 18-3 (50 mg, yield 17.7%) as a yellow solid. MS m/z: 746 [M+H]⁺.

To a solution of Compound 18-3 (50 mg, 0.07 mmol) in DCM (2 mL) was added HCl (4 M in 1,4-dioxane, 0.4 mL) at room temperature and stirred for 1 h. Then the reaction mixture was concentrated to give Compound 18-4 (50 mg, crude) as a yellow solid. MS m/z: 702 [M+H]⁺.

To a solution of Compound 18-4 (50 mg crude, 0.07 mmol) in DMF (1 mL) was added CsF (162 mg, 1.07 mmol) at room temperature. The reaction mixture was heated to 40° C. and stirred for 12 h. Then the reaction mixture was filtered, and the filtrate was purified by Prep-HPLC to give Compound 18 (HCOOH salt, 3.5 mg, yield 8.8% for two steps) as a yellow solid.

¹H NMR (400 MHz, CD₃OD): δ 9.15 (s, 1H), 8.44 (brs, 1H), 7.87 (s, 1H), 7.36-7.30 (m, 2H), 7.20 (s, 1H), 5.56-5.43 (m, 1H), 4.66-4.62 (m, 2H), 3.89-3.65 (m, 3H), 3.62 (s, 2H), 3.20 (s, 1H), 2.54-2.49 (m, 2H), 2.36-2.25 (m, 4H), 0.98-0.97 (m, 2H), 0.82 (s, 2H). MS m/z: 546 [M+H]⁺.

### Example 20

### 5-ethynyl-6-fluoro-4-(8-fluoro-2-(((2R,7aS)-2-fluorotetrahydro-1H-pyrrolizin-7a(5H)-yl)methoxy)-4-(methyl(1-methylcyclopropyl)amino)pyrido[4,3-d]pyrimidin-7-1 naphthalen-2-ol (“Compound 19”)

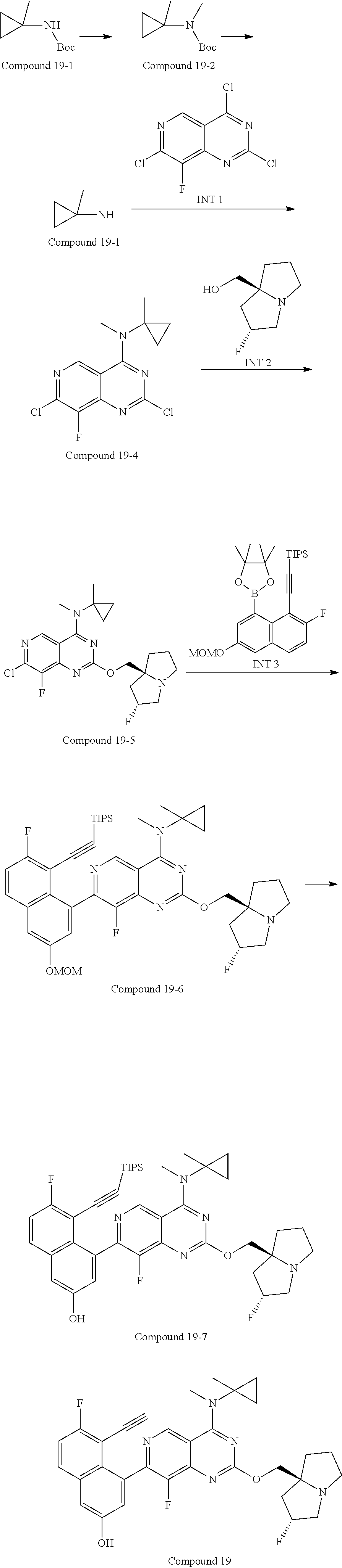

NaH (112 mg, 2.8 mmol) was added to a DMF solution of Compound 19-1 (400 mg, 2.34 mmol) at 0° C. under N₂ atmosphere. The mixture was stirred for 1 h. After that, the mixture was added Mel (400 mg, 2.8 mmol) and stirred overnight. After the completion of reaction, the mixture was poured into water (5 mL) and extracted with EtOAc (5 mL). The organic layer was washed with water (5 mL), brine (5 mL), dried with anhydrous Na₂SO₄ and filtered. The filtrate was concentrated to dryness. The residue was purified by column (eluted with Petroleum ether/EtOAc=3:1) to give Compound 19-2 (400 mg, yield 92.5%).

¹H NMR (300 MHz, CDCl₃): δ 1.43 (s, 9H), 1.33 (s, 3H), 1.25 (t, J=6.9 Hz, 3H), 0.71 (s, 2H), 0.56 (s, 2H).

To a solution of Compound 19-2 (400 mg, 2.1 mmol) in DCM (2.0 mL) was added HCl/dioxane (2.0 mL) at room temperature. The mixture was stirred for 2 h. Then the reaction mixture was concentrated to give Compound 19-3 (150 mg, crude).

¹H NMR (300 MHz, CDCl₃): δ 1.42 (s, 3H), 1.12 (s, 3H), 0.83-0.75 (m, 2H), 0.63 (t, J=6.0 Hz, 2H).

Compound 19-3 (300 mg, 1.20 mmol) and DIEA (1.0 g, 7.9 mmol) were added successively into DCM (5 mL) under N₂ atmosphere. The mixture was cooled to −40° C. and INT 1 (114 mg, 1.34 mmol) was added. The resulting mixture was stirred for 1 h. After that, the mixture was poured into water (5 mL) and extracted with DCM (5 mL). The organic layer was washed with water (5 mL), brine (5 mL), dried with anhydrous Na₂SO₄ and filtered. The filtrate was concentrated to dryness. The residue was purified by Pre-TLC (eluted with Petroleum ether/EtOAc=3:1) to give Compound 19-4 (200 mg, yield 55.8%). MS m/z: 301 [M+H]⁺.

Compound 19-4 (200 mg, 0.66 mmol), INT 2 (211 mg, 1.32 mmol), DIEA (260 mg, 1.9 mmol) and 4A MS were added successively into dioxane (6 mL) under N₂ atmosphere. The mixture was heated to 85° C. and stirred overnight. After that, the mixture was cooled to room temperature, poured into water (10 mL) and extracted with DCM (8 mL×2). The organic layers were washed with water (5 mL), brine (5 mL), dried with anhydrous Na₂SO₄ and filtered. The filtrate was concentrated to dryness. The residue was purified by Pre-TLC (eluted with Petroleum ether/EtOAc=1:1) to give Compound 19-5 (110 mg, yield 39.1%). MS m/z: 424 [M+H]⁺.

Compound 19-5 (90 mg, 0.21 mmol), INT 3 (200 mg, 0.4 mmol), Cs₂CO₃ (190 mg, 0.6 mmol) and Cxiuma-Pd (15 mg, 0.02 mmol) were added successively into toluene/water=5:1 (2.4 mL). The reaction mixture was degassed with argon for 2 min. The mixture was heated to 105° C. under N₂ atmosphere and stirred for 2 h in microwave. After that, the mixture was cooled to room temperature, poured into water (5 mL) and extracted with EtOAc (5 mL×2). The organic layer was washed with water (5 mL), brine (5 mL), dried with anhydrous Na₂SO₄ and filtered. The filtrate was concentrated to dryness. The residue was purified by Pre-TLC (eluted with DCM/MeOH=10:1) to give Compound 19-6 (80 mg, yield 48.7%). MS m/z: 774 [M+H]⁺.

To a solution of Compound 19-6 (80 mg, 0.1 mmol) in DCM (2.0 mL) was added HCl (4 M in 1,4-dioxane, 2.0 mL) at room temperature. The reaction was stirred for 2 h and then concentrated to give Compound 19-7 (75 mg, crude). MS m/z: 730 [M+H]⁺.

To a solution of Compound 19-7 (75 mg crude, 0.1 mmol) in DMF (3 mL) was added CsF (308 mg, 2.0 mmol) at room temperature. The reaction mixture was heated to 40° C. and stirred for 4 h. Then the reaction mixture was filtered and the filtrate was purified by Prep-HPLC to give Compound 19 (23.3 mg, yield 39.3% for two steps).

¹H NMR (300 MHz, CD₃OD): δ 9.41 (s, 1H), 8.46 (brs, 1H), 7.89-7.84 (m, 1H), 7.36-7.30 (m, 2H), 7.22 (d, J=2.4 Hz, 1H), 5.55-5.31 (m, 1H), 4.53-4.47 (m, 2H), 3.79-3.65 (m, 3H), 3.62-3.56 (m, 3H), 3.45 (s, 1H), 3.32-3.25 (m, 1H), 2.60-2.47 (m, 2H), 2.39-2.15 (m, 3H), 2.05 (s, 1H), 1.77 (s, 3H), 1.16 (s, 4H). MS m/z: 574 [M+H]⁺.

### Example 21

### 5-ethynyl-6-fluoro-4-(8-fluoro-4-(((1S,2S)-2-fluorocyclopropyl)(methyl)amino)-2-(((2R,7aS)-2-fluoro tetrahydro-1H-pyrrolizin-7a(5H)-yl)methoxy)pyrido[4,3-d]pyrimidin-7-yl)naphthalen-2-yl methylcarbamate 2,2,2-trifluoroacetic acid (“Compound 20 (TFA salt)”)

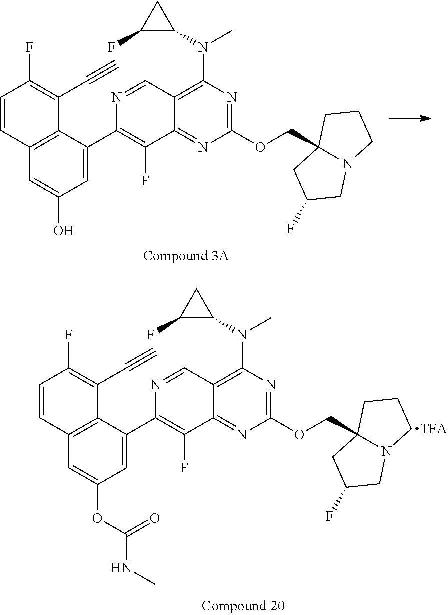 [SMILES: C#Cc1c(F)ccc2cc(O)cc(-c3ncc4c(N(C)[C@H]5C[C@@H]5F)nc(OC[C@@]56CCCN5C[C@H](F)C6)nc4c3F)c12 ; C#Cc1c(F)ccc2cc(OC(=O)NC)cc(-c3ncc4c(N(C)[C@H]5C[C@@H]5F)nc(OC[C@@]56CCCN5C[C@H](F)C6)nc4c3F)c12]

To a solution of Compound 3A (99 mg, 0.17 mmol) and DIEA (1 mL) in DCM (10 mL) was added methylcarbamic chloride (99 mg, 1.06 mmol). The reaction mixture was stirred at room temperature for 2 hours. The reaction mixture was diluted with saturated aqueous NH₄Cl solution (20 mL) and extracted with DCM (20 mL). The organic layer was dried over anhydrous Na₂SO₄ and concentrated under reduced pressure.

The crude product was purified by Pre-HPLC with the following condition (C18 column, A: 0.1% TFA in water, B: CH₃CN, Gradient: 15% B to 50% B in 38 min at a flow rate of 60 mL/min, 230 nm) and the product fractions were lyophilized to give Compound 20 (TFA salt) (75.4 mg, 0.10 mmol, 58.8% yield). MS m/z: 635 [M+H]⁺.

### Example 22

### 5-ethynyl-6-fluoro-4-(8-fluoro-4-(((1S,2S)-2-fluorocyclopropyl)(methyl)amino)-2-(((2R,7aS)-2-fluoro tetrahydro-1H-pyrrolizin-7a(5H)-yl)methoxy)pyrido[4,3-d]pyrimidin-7-yl)naphthalen-2-yl dimethylcarbamate-2,2,2-trifluoroacetic acid_(“Compound 21 (TFA salt)”)

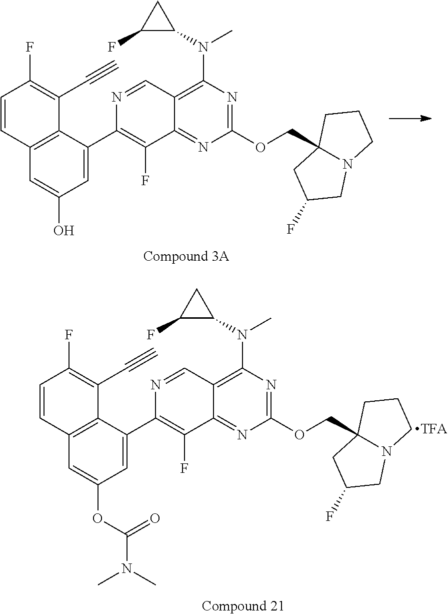 [SMILES: C#Cc1c(F)ccc2cc(O)cc(-c3ncc4c(N(C)[C@H]5C[C@@H]5F)nc(OC[C@@]56CCCN5C[C@H](F)C6)nc4c3F)c12 ; C#Cc1c(F)ccc2cc(OC(=O)N(C)C)cc(-c3ncc4c(N(C)[C@H]5C[C@@H]5F)nc(OC[C@@]56CCCN5C[C@H](F)C6)nc4c3F)c12]

To a solution of Compound 3A (109.2 mg, 0.19 mmol) and pyridine (2 mL) in acetonitrile (10 mL) was added dimethylcarbamic chloride (3 drops). The reaction mixture was stirred at room temperature overnight. The reaction mixture was diluted with saturated aqueous NH₄Cl solution (20 mL) and extracted with EtOAc (20 mL). The organic layer was dried over anhydrous Na₂SO₄ and concentrated under reduced pressure. The crude product was purified by Pre-HPLC (C18 column, A: 0.1% TFA in water, B: CH₃CN, Gradient: 15% B to 50% B in 40 min at a flow rate of 60 mL/min, 230 nm) and the product fractions were lyophilized to give Compound 21 (TFA salt) (88 mg, 0.12 mmol, 61.0% yield). MS m/z: 649 [M+H]⁺.

### Example 23

### 5-ethynyl-6-fluoro-4-(8-fluoro-2-(((2R,7aS)-2-fluorotetrahydro-1H-pyrrolizin-7a(5H)-yl)methoxy)-4-((2-methylcyclopropyl)amino)pyrido[4,3-d]pyrimidin-7-yl)naphthalen-2-ol (“Compound 22”)

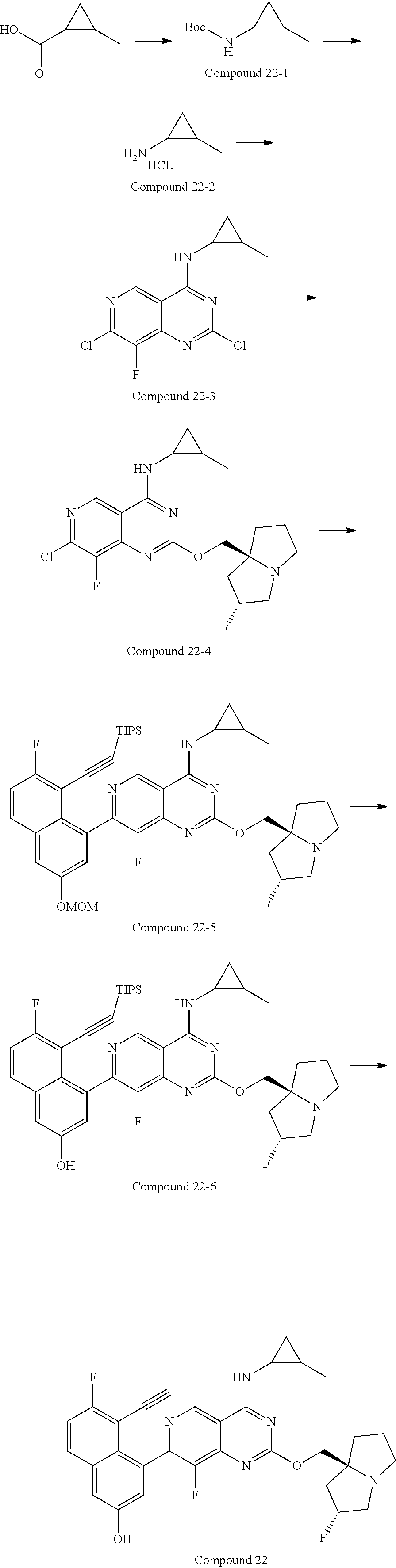

A solution of 2-methylcyclopropanecarboxylic acid (295 mg, 2.9466 mmol), DPPA (784 mg, 3.2237 mmol) and TEA (349 mg, 3.4490 mmol) in tert-butanol (10 mL) was stirred for 8 hours at 80° C. under nitrogen atmosphere. The solution was concentrated under vacuum to give crude Compound 22-1 (702 mg, 4.0996 mmol, 139.1301% yield). MS m/z: 172 [M+H]⁺.

A solution of Compound 22-1 (0.702 g, 4.0996 mmol) and hydrogen chloride (4 M in 1,4-dixoane, 2 mL) in DCM (10 mL) was stirred at room temperature for 20 h. The solution was concentrated under vacuum to give Compound 22-2 (1397 mg, 12.9855 mmol, 316.7498% yield). MS m/z: 72 [M+H]⁺.

A solution of 2,4,7-trichloro-8-fluoropyrido[4,3-d]pyrimidine (311 mg, 1.2319 mmol), N,N-diisopropylethylamine (504 mg, 3.8996 mmol) and Compound 22-2 (837 mg, 7.7801 mmol) in DCM (10 mL) was stirred 4 h at room temperature. The solution was diluted with 10% citric acid solution (10 mL). The organic layer was washed with 10 mL aqueous NaCl solution, dried over anhydrous Na₂SO₄ and concentrated in vacuum to give Compound 22-3 (290 mg, 1.0100 mmol, 81.9912%). MS m/z: 287 [M+H]⁺.

A solution of INT 2 (241.1964 mg, 1.5150 mmol), Compound 22-3 (0.29 g, 1.0100 mmol) and potassium fluoride (176.0383 mg, 3.0301 mmol) in DMSO (10 mL) was stirred at 100° C. for 16 h under nitrogen atmosphere. The mixture was allowed to cool to room temperature and diluted with water (20 mL) and extracted with EA (20 mL). The organic layer was washed with 10 mL aqueous NaCl solution, dried over anhydrous Na₂SO₄ and concentrated in vacuum. Then the residue purified by TLC to give Compound 22-4 (213 mg, 519.6899 μmol, 51.4529% yield). MS m/z: 410 [M+H]⁺.

A solution of Compound 22-4 (213 mg, 519.6899 μmol), toluene (5 mL), INT 3 (384 mg, 749.2177 mol), cataCXium A Pd G3 (39 mg, 53.5516 μmol), cesium carbonate (519 mg, 1.5929 mmol) and water (1 mL) was stirred at 100° C. for 14 hours under nitrogen atmosphere. The mixture was allowed to cool to room temperature, diluted with water (15 mL) and extracted with EA (15 mL). The organic layer was washed with aqueous NaCl solution (10 mL) then dried over anhydrous Na₂SO₄ and concentrated in vacuum. The residue was purified by Pre-TLC to give Compound 22-5 (313 mg, 411.8570 μmol, 79.2506% yield). MS m/z: 760 [M+H]⁺.

A solution of Compound 22-5 (313 mg, 411.8570 μmol) and HCl (4 M in 1,4-dioxane, 1 mL) in DCM (10 mL) was stirred at room temperature for 2 h. The solution was diluted with 10% NaHCO₃ solution (10 mL) and extracted with DCM (10 mL). The organic layer was washed with aqueous NaCl solution, dried over anhydrous Na₂SO₄ and concentrated in vacuum to give crude Compound 22-6 (236 mg, 329.6457 μmol, 80.0389% yield). MS m/z: 716 [M+H]⁺.

A solution of Compound 22-6 (236 mg, 329.6457 μmol) and CsF (233 mg, 1.5339 mmol) in DMF (10 mL) was stirred at room temperature for 20 hours under nitrogen atmosphere. The reaction mixture was diluted with water (10 mL) and extracted with EA (10 mL). The organic layer was washed with saturated aqueous NaCl solution (10 mL), dried over anhydrous Na₂SO₄ and concentrated under vacuum. The residue was purified by Prep-HPLC (C18 column, A: 0.1% TFA in water, B: CH₃CN, Gradient: 15% B to 15% B in 2 min, 15% B to 21% B in 13 min at a flow rate of 60 mL/min, 270 nm). The eluent was adjusted to pH=8. The acetonitrile in the eluent was concentrated. The resulting aqueous layer was extracted with EA and then the organic layer was dried, concentrated and lyophilized to give Compound 22 (79 mg, 141.1772 μmol, 42.8269% yield). MS m/z: 560 [M+H]⁺.

### Example 24

### Di-tert-butyl (((5-ethynyl-6-fluoro-4-(8-fluoro-4-((trans-2-fluorocyclopropyl)(methyl)amino)-2-(((2R,7aS)-2-fluorotetrahydro-1H-pyrrolizin-7a(5H)-yl)methoxy)pyrido[4,3-d]pyrimidin-7-yl)naphthalen-2-yl)oxy)methyl) phosphate (“Compound 23”)

### ((5-ethynyl-6-fluoro-4-(8-fluoro-4-((trans-2-fluorocyclopropyl)(methyl)amino)-2-(((2R,7aS)-2-fluoro tetrahydro-1H-pyrrolizin-7a(5H)-yl)methoxy)pyrido[4,3-d]pyrimidin-7-yl)naphthalen-2-yl)oxy)methyl dihydrogen phosphate 2,2,2-trifluoroacetic acid (“Compound 24 (TFA salt)”)

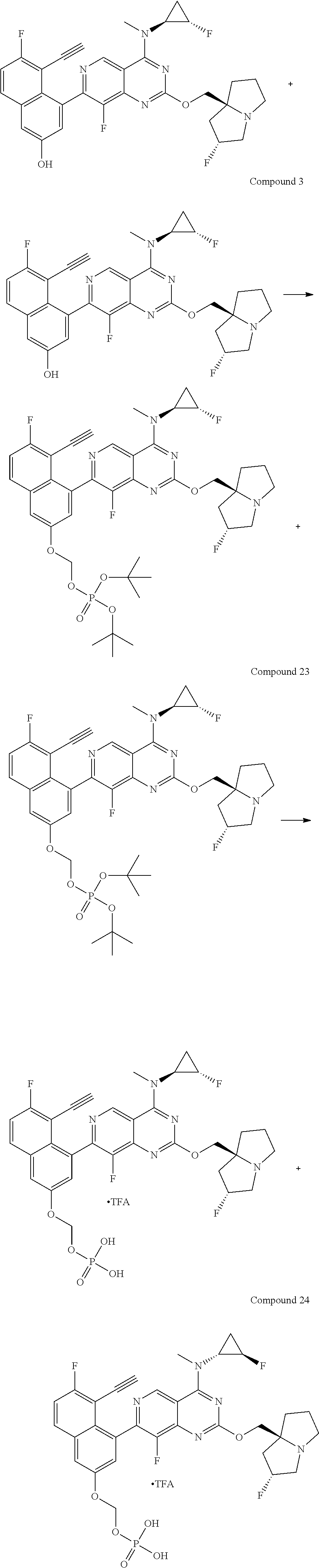

A solution of 2-[tert-butoxy(chloromethyl)phosphoryl]oxy-2-methyl-propane (0.62 g, 2.5548 mmol), Compound 3 (1.02 g, 1.7660 mmol) and potassium carbonate (0.75 g, 5.4267 mmol) in N,N-dimethylformamide (20 mL) was stirred at 60° C. for 18 h. The reaction mixture was concentrated under vacuum. The residue was purified by Prep-HPLC (C18 column, A: 0.05% TFA in water, B: CH₃CN, Gradient: 25% B to 80% B in 60 min at a flow rate of 200 mL/min, 254 nm). The eluent was adjusted to pH=8 with saturated NaHCO₃. The acetonitrile in the eluent was concentrated. The resulting aqueous layer was extracted with EA (200 mL×2) and then the organic layer was dried, concentrated and lyophilized to give Compound 23 (514 mg, 642.6697 μmol 36.3909% yield). MS m/z: 800 [M+H]⁺.

A solution of Compound 23 (0.31 g, 387.6022 μmol) and trifluoroacetic acid (2 mL) in dichloromethane (10 mL) was stirred at room temperature for 5 h. The reaction mixture was concentrated under vacuum. The residue was purified by Prep-HPLC (C18 column, A: 0.05% TFA in water, B: CH₃CN, Gradient: 20% B to 50% B in 60 min at a flow rate of 70 mL/min, 242 nm) and lyophilized to give Compound 24 (TFA salt) (228 mg, 284.4312 μmol, 73.3823% yield). MS m/z: 688 [M+H]⁺.

### Example 25

### 5-ethynyl-6-fluoro-4-(8-fluoro-4-((1-(fluoromethyl)cyclopropyl)amino)-2-(((2R,7aS)-2-fluorotetrahydro-1H-pyrrolizin-7a(5H)-yl)methoxy)pyrido[4,3-d]pyrimidin-7-yl)naphthalen-2-ol (“Compound 25”)

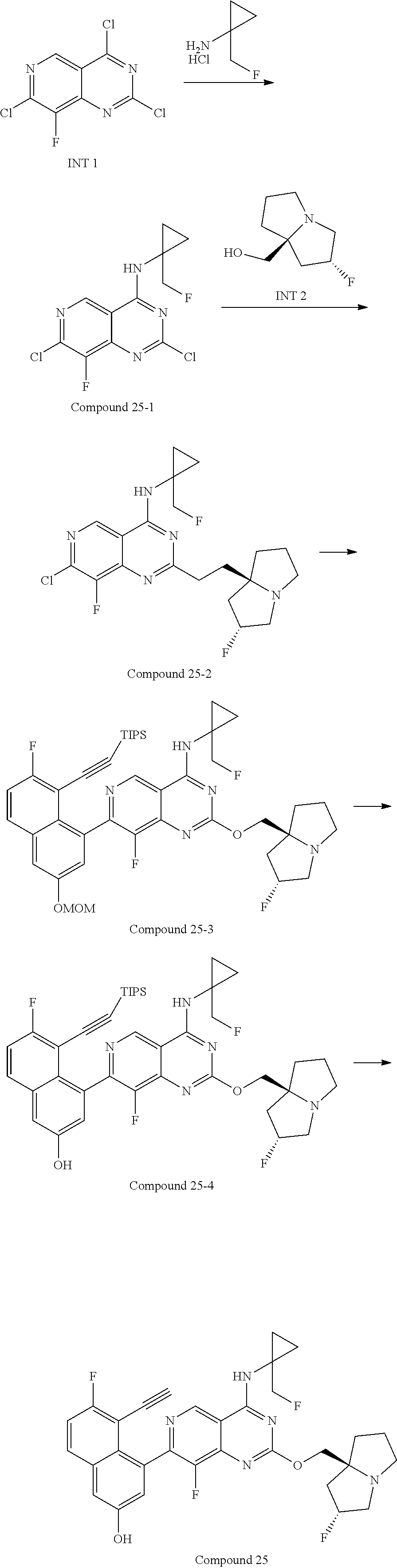

1-(fluoromethyl)cyclopropanamine (98 mg, 780.4281 μmol) was added into a solution of 2,4,7-trichloro-8-fluoropyrido[4,3-d]pyrimidine (210 mg, 831.8140 μmol) and N,N-disopropylethylamine (327 mg, 2.5301 mmol) in DCM (10 mL) at 0° C. Then the solution was stirred at room temperature for 3 h. The solution was diluted with 10% citric acid solution (50 mL). The organic layer was washed with aqueous NaCl solution, dried over anhydrous Na₂SO₄ and concentrated in vacuum to give Compound 25-1 (303 mg, 993.0828 μmol, 119.3876% yield). MS m/z: 305 [M+H]⁺.

A solution of Compound 25-1 (303 mg, 993.0828 μmol), INT 2 (237 mg, 1.4887 mmol) and KF (186 mg, 3.2016 mmol) in DMSO (10 mL) was stirred at 85° C. for 20 h under nitrogen atmosphere. The mixture was allowed to cool to room temperature, diluted with water (10 mL) and extracted with EA (10 mL). The organic layer was washed with 10 mL aqueous NaCl solution then dried over anhydrous Na₂SO₄ and concentrated in vacuum. The residue was purified by Pre-TLC to give Compound 25-2 (340 mg, 794.6705 μmol, 80.0207% yield. MS m/z: 428 [M+H]⁺.

A solution of Compound 25-2 (340 mg, 794.6705 μmol), toluene (7.5 mL), INT 3 (619 mg, 1.2077 mmol), cataCXium A Pd G3 (77 mg, 105.7300 μmol), Potassium phosphate (760 mg, 2.3326 mmol) and water (1.5 mL) was stirred at 105° C. for 3 hours under nitrogen atmosphere. The mixture was allowed to cool to room temperature and diluted with water (15 mL) and extracted with EA (15 mL). The organic layer was washed with 10 mL aqueous NaCl solution then dried over anhydrous Na₂SO₄ and concentrated in vacuum. The residue was purified by Pre-TLC to give Compound 25-3 (312 mg, 401.0473 μmol, 50.4671% yield). MS m/z: 778 [M+H]⁺.

A solution of Compound 25-3 (312 mg, 401.0473 μmol) and HCl (4 M in 1,4-dioxane, 1 mL) in DCM (10 mL) was stirred at room temperature for 1 h. The solution was diluted with 10% NaHCO₃ solution (20 mL). The organic layer was washed with saturated aqueous NaCl solution, dried over anhydrous Na₂SO₄ and concentrated in vacuum to give crude Compound 25-4 (325 mg, 442.8332 μmol, 110.4192% yield). MS m/z: 734 [M+H]⁺.

A solution of Compound 25-4 (325 mg, 442.8332 μmol) and CsF (301 mg, 1.9815 mmol) in DMF (10 mL) was stirred for 20 hours at 40° C. under nitrogen atmosphere. The solution was diluted with water (10 mL) and extracted with EA (10 mL). The organic layer was dried over anhydrous Na₂SO₄ and concentrated under vacuum. The residue was purified by Prep-HPLC (C18 column, A: 0.1% TFA in water, B: CH₃CN, Gradient: 10% B to 44% B in 55 min at a flow rate of 60 mL/min, 234 nm). The pH of the eluent was adjusted to 8 and the acetonitrile in the eluent was concentrated. The aqueous layer was extracted with EA and the resulting organic layer was dried, concentrated and lyophilized to give Compound 25 (75 mg, 129.8542 μmol, 29.3235% yield). MS m/z: 578 [M+H]⁺.

### Example 26

### 4-(4-(cyclopropyl(methyl-d3)amino)-8-fluoro-2-(((2R,7aS)-2-fluorotetrahydro-1H-pyrrolizin-7a(5H)-yl-2,5,5-d3)methoxy-d2)pyrido[4,3-d]pyrimidin-7-yl)-5-ethynyl-6-fluoronaphthalen-2-ol (“Compound 26”)

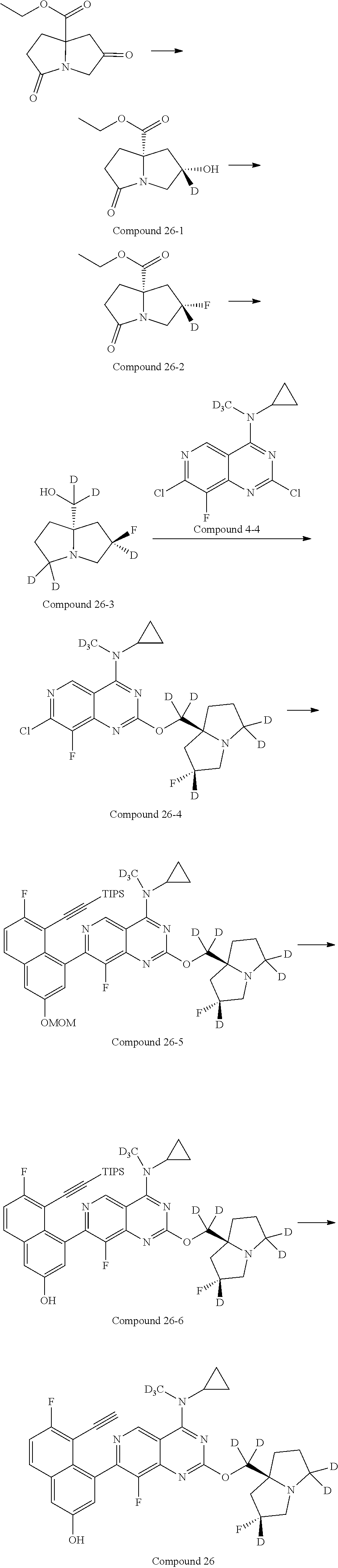

A solution of ethyl 2,5-dioxotetrahydro-1H-pyrrolizine-7a(5H)-carboxylate (2172 mg, 10.2834 mmol) in EtOH (12 mL) was added NaBD₄ (160 mg, 3.8225 mmol) at 0° C. The reaction mixture was stirred at room temperature for 0.5 h. The solution was quenched with water (0.5 mL) and concentrated in vacuum. The residue was purified by normal phase chromatography (silica gel cartridge, hexane/EA=1:1) to give Compound 26-1 (1485 mg, 6.8992 mmol, 67.0906% yield). MS: m/z: 215 [M+H]⁺.

A solution of Compound 26-1 (1485 mg, 6.8992 mmol) in DCM (10 mL) was added DAST (1.62 g, 10.0503 mmol) at −78° C. The reaction mixture was stirred at room temperature for 4 h. The solution was quenched with MeOH (0.5 mL) at 0° C., diluted with water (10 mL) and extracted with DCM (10 mL×3). The organic layer was washed with aqueous NaCl solution (20 mL), dried over anhydrous Na₂SO₄ and concentrated in vacuum. The residue was purified by normal phase chromatography (silica gel cartridge, hexane/EA=1:1) to give Compound 26-2 (1103 mg, 5.4550 mmol, 79.0668% yield). MS: m/z: 217 [M+H]⁺.

A solution of Compound 26-2 (1103 mg, 5.4550 mmol) in THF (10 mL) was added LiAlD₄ (414 mg, 8.2730 mmol) at 0° C. The reaction mixture was stirred at 70° C. for 3 h. The solution was quenched with water (0.4 mL), 15% sodium hydroxide solution (0.4 mL) and water (1.2 mL), filtered and concentrated in vacuum.

The residue was purified by normal phase chromatography (silica gel cartridge, DCM/MeOH=10:1) to give Compound 26-3 (707 mg, 4.3049 mmol, 78.9168% yield). MS: m/z: 165 [M+H]⁺.

A solution of Compound 26-3 (212 mg, 730.6853 μmol), Compound 4-4 (175 mg, 1.0656 mmol) and KF (254 mg, 4.3720 mmol) in DMSO (10 mL) was stirred at 100° C. for 16 h under nitrogen atmosphere. The mixture was allowed to cool to room temperature, diluted with saturated NaHCO₃ aqueous solution (50 mL) and extracted with EA (2×30 mL). The organic layer was washed with aqueous NaCl solution (50 mL×2) then dried over anhydrous Na₂SO₄ and concentrated in vacuum. The residue was purified by Pre-TLC (DCM/MeOH=15:1) to give Compound 26-4 (158 mg, 378.0724 μmol, 51.7422% yield). MS: m/z: 418 [M+H]⁺.

A solution of Compound 26-4 (158 mg, 378.0724 μmol), toluene (10 mL), INT-3 (340 mg, 663.3693 μmol), cataCXium A Pd G3 (82 mg, 112.5956 μmol)), cesium carbonate (416 mg, 1.2768 mmol) and water (2 mL) was stirred at 100° C. for 16 hours under nitrogen atmosphere. The mixture was allowed to cool to room temperature, diluted with saturated NaHCO₃ aqueous solution (50 mL) and extracted with DCM (2×50 mL). The organic layer was washed with 50 ml aqueous NaCl solution, dried over anhydrous Na₂SO₄ and concentrated in vacuum. The residue was purified by Pre-TLC (DCM/MeOH=15:1) to give Compound 26-5 (203 mg, 264.3153 μmol, 69.9113% yield). MS: m/z: 768 [M+H]⁺.

A solution of Compound 26-5 (203 mg, 264.3153 μmol) and HCl (1 mL, 4 M in dioxane) in DCM (5 mL) was stirred at room temperature for 1 h. The solution was diluted with 10% NaHCO₃ solution (50 mL) and extracted with DCM (2×30 mL). The organic layer was washed with saturated aqueous NaCl solution (50 mL), dried over anhydrous Na₂SO₄ and concentrated in vacuum to give crude Compound 26-6 (279 mg, 385.3752 μmol, 145.8013% yield). MS: m/z: 724 [M+H]⁺.

A solution of Compound 26-6 (279 mg, 385.3752 μmol) and CsF (1226 mg, 8.0709 mmol) in DMF (10 mL) was stirred for 16 hours at 35° C. under nitrogen atmosphere. The solution was diluted with saturated aqueous NaHCO₃ solution (50 mL) and extracted with EA (2×30 mL). The organic layer was washed with saturated aqueous NaCl solution (50 mL×2), dried over anhydrous Na₂SO₄ and concentrated under vacuum. The residue was purified by Prep-HPLC (C18 column, A: 0.1% TFA in water, B: CH₃CN, Gradient: 15% B to 45% B in 40 min at a flow rate of 60 mL/min, 240 n). The eluent was adjusted to pH=8 with saturated NaHCO₃. The acetonitrile in the eluent was concentrated. The resulting aqueous layer was extracted with EA (100 mL×2) and then the organic layer was dried, concentrated and lyophilized to give Compound 26 (68 mg, 119.7963 μmol, 31.0856% yield). MS: m/z: 568 [M+H]⁺.

### Example 27

### 5-ethynyl-6-fluoro-4-(8-fluoro-4-(((1S,2S)-2-fluorocyclopropyl)(methyl)amino)-2-(((2R,7aS)-2-fluoro tetrahydro-1H-pyrrolizin-7a(5H)-yl)methoxy)pyrido[4,3-d]pyrimidin-7-yl)naphthalen-2-yl sulfamate 2,2,2-trifluoroacetic acid (“Compound 27 (TFA salt)”)

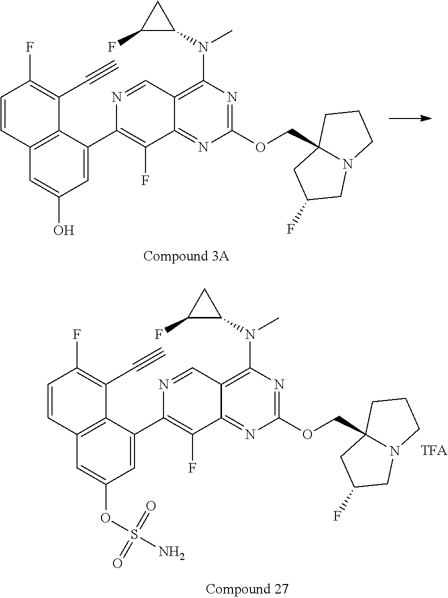 [SMILES: C#Cc1c(F)ccc2cc(O)cc(-c3ncc4c(N(C)[C@H]5C[C@@H]5F)nc(OC[C@@]56CCCN5C[C@H](F)C6)nc4c3F)c12 ; C#Cc1c(F)ccc2cc(OS(N)(=O)=O)cc(-c3ncc4c(N(C)[C@H]5C[C@@H]5F)nc(OC[C@@]56CCCN5C[C@H](F)C6)nc4c3F)c12]

To a solution of chlorosulfonyl isocyanate (7.5 mL) in acetonitrile (12 mL) was added formic acid (3.3 mL) dropwise at 0° C. The mixture was stirred at 0° C. for 1 hour, then diluted with acetonitrile (24 mL). The mixture was stirred at room temperature for 3.5 hours. Then 2 mL of the solution was treated in another flask and stirred at 0° C., then Compound 3A (50 mg, 0.086 mmol) in DMAc (2 mL) was added dropwise. The mixture was stirred at room temperature for 16 hours. The reaction mixture was diluted with water (20 mL) and extracted with EtOAc (2×20 mL). The combined organic layers were dried over anhydrous Na₂SO₄ and concentrated under reduced pressure. The crude product was purified by Pre-HPLC with the following condition (Ultimate XB—C18, 30 mm×150 mm, 5 um; A: 0.1% TFA in water, B: CH₃CN, Gradient: 10% B to 36% B in 60 min at a flow rate of 40 mL/min, 242 nm) and the product fractions were lyophilized to give Compound 27 (TFA salt, 37.9 mg). MS: m/z: 657[M+H]⁺.

### Example 28

### ethyl2-((5-ethynyl-6-fluoro-4-(8-fluoro-4-(((1S,2S)-2-fluorocyclopropyl)(methyl)amino)-2-(((2R,7aS)-2-fluorotetrahydro-1H-pyrrolizin-7a(5H)-yl)methoxy)pyrido[4,3-d]pyrimidin-7-yl)naphthalen-2-yl)oxy) acetate 2,2,2-trifluoroacetic acid (“Compound 28 (TFA salt”)

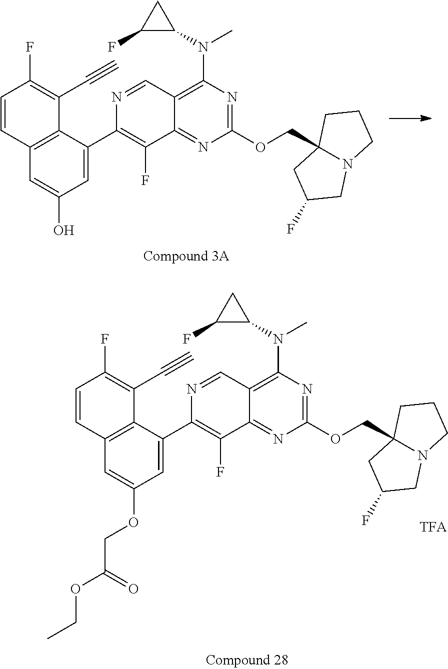 [SMILES: C#Cc1c(F)ccc2cc(O)cc(-c3ncc4c(N(C)[C@H]5C[C@@H]5F)nc(OC[C@@]56CCCN5C[C@H](F)C6)nc4c3F)c12 ; C#Cc1c(F)ccc2cc(OCC(=O)OCC)cc(-c3ncc4c(N(C)[C@H]5C[C@@H]5F)nc(OC[C@@]56CCCN5C[C@H](F)C6)nc4c3F)c12]

A solution of Compound 3A (151 mg, 0.26 mmol), K₂CO₃ (169 mg, 1.22 mmol) and ethyl 2-bromoacetate (44 mg, 0.26 mmol) in DMF (5 mL) was stirred at 80° C. for 4 hours. The reaction mixture was diluted with water (20 mL) and extracted with EtOAc (20 mL). The organic layer was dried over anhydrous Na₂SO₄ and concentrated under reduced pressure. The crude product was purified by Pre-HPLC with the following condition (Daisogel C18, 50 mm×250 mm, 10 um; A: 0.1% TFA in water, B: CH₃CN, Gradient: 15% B to 59% B in 48 min at a flow rate of 60 mL/min, 265 nm) and freeze-dried to give Compound 28 (TFA salt, 44.1 mg, 21.69% yield). MS: m/z: 664[M+H]⁺.

### Example 29

### 5-ethynyl-6-fluoro-4-(8-fluoro-4-(((1S,2S)-2-fluorocyclopropyl)(methyl)amino)-2-(((2R,7aS)-2-fluoro tetrahydro-1H-pyrrolizin-7a(5H)-yl)methoxy)pyrido[4,3-d]pyrimidin-7-yl)naphthalen-2-yl (2-acetamidoethyl)(methyl)carbamate 2,2,2-trifluoroacetic acid (“Compound 29 (TFA salt)”)

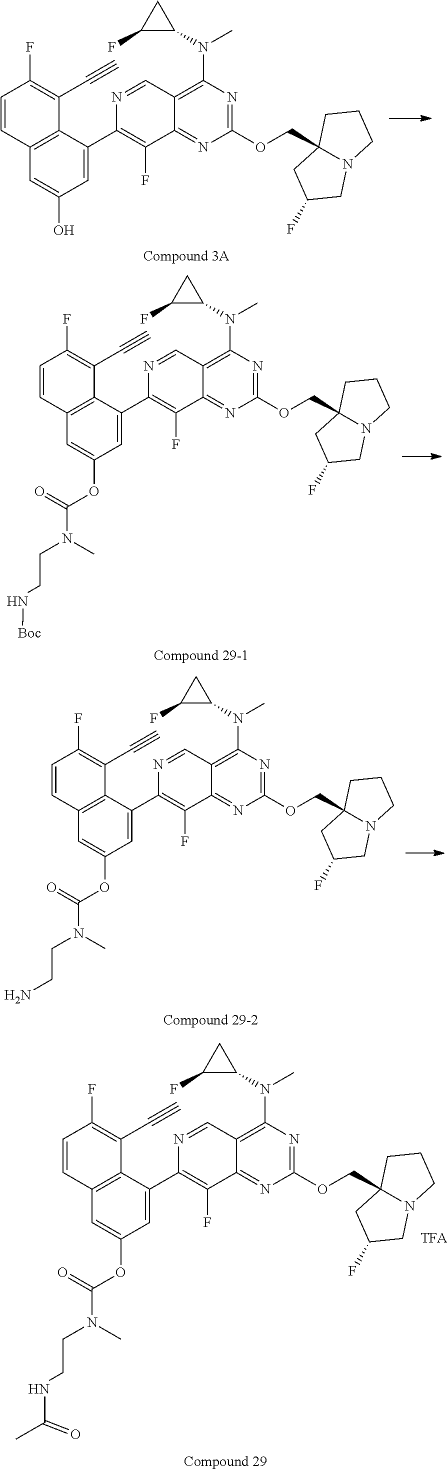 [SMILES: C#Cc1c(F)ccc2cc(O)cc(-c3ncc4c(N(C)[C@H]5C[C@@H]5F)nc(OC[C@@]56CCCN5C[C@H](F)C6)nc4c3F)c12 ; C#Cc1c(F)ccc2cc(OC(=O)N(C)CCNC(=O)OC(C)(C)C)cc(-c3ncc4c(N(C)[C@H]5C[C@@H]5F)nc(OC[C@@]56CCCN5C[C@H](F)C6)nc4c3F)c12 ; C#Cc1c(F)ccc2cc(OC(=O)N(C)CCN)cc(-c3ncc4c(N(C)[C@H]5C[C@@H]5F)nc(OC[C@@]56CCCN5C[C@H](F)C6)nc4c3F)c12 ; C#Cc1c(F)ccc2cc(OC(=O)N(C)CCNC(C)=O)cc(-c3ncc4c(N(C)[C@H]5C[C@@H]5F)nc(OC[C@@]56CCCN5C[C@H](F)C6)nc4c3F)c12]

To a solution of Compound 3A (404 mg, 0.66 mmol) and DIEA (1 mL) in DCM (10 mL) was added 4-nitrophenyl carbonochloridate (137 mg, 0.79 mmol). The resulting mixture was stirred for 25 minutes at room temperature, then tert-butyl (2-(methylamino)ethyl)carbamate (137 mg, 0.79 mmol) was added and stirred for 2 hours at room temperature. The mixture was diluted with water (20 mL) and the organic layer was separated, dried over anhydrous Na₂SO₄ and concentrated under reduced pressure. The residue was purified by Pre-TLC to give Compound 29-1 (598 mg, 116.85% yield). MS: m/z: 778[M+H]⁺.

To a solution of Compound 29-1 (598 mg, 0.77 mmol) in DCM (10 mL) was added TFA (3 mL). The resulting mixture was stirred for 2.5 hours at room temperature, then concentrated under reduced pressure to give Compound 29-2. MS: m/z: 678[M+H]⁺.

A solution of Compound 29-2 (260 mg, 0.38 mmol), acetic anhydride (1 mL) and DIEA (2 mL) in DCM (5 mL) was stirred at room temperature for 2 hours. The reaction mixture was diluted with DCM (20 mL) and washed with water (20 mL) and sat. NH₄Cl (20 mL). The organic layer was dried over anhydrous Na₂SO₄ and concentrated under reduced pressure. The crude product was purified by Pre-HPLC with the following condition (Daisogel C18, 50 mm×250 mm, 10 um; A: 0.1% TFA in water, B: CH₃CN, Gradient: 15% B to 50% B in 40 min at a flow rate of 60 mL/min, 245 nm) and freeze-dried to give Compound 29 (TFA salt, 162.2 mg, 25.30% yield). MS: m/z: 720[M+H]⁺.

### Example 30

### 5-ethynyl-6-fluoro-4-(8-fluoro-4-(((1S,2S)-2-fluorocyclopropyl)(methyl)amino)-2-(((2R,7aS)-2-fluoro tetrahydro-1H-pyrrolizin-7a(5H)-yl)methoxy)pyrido[4,3-d]pyrimidin-7-yl)naphthalen-2-yl methyl(2-(2,2,2-trifluoroacetamido)ethyl)carbamate 2,2,2-trifluoroacetic acid (“Compound 30 (TFA salt)”)

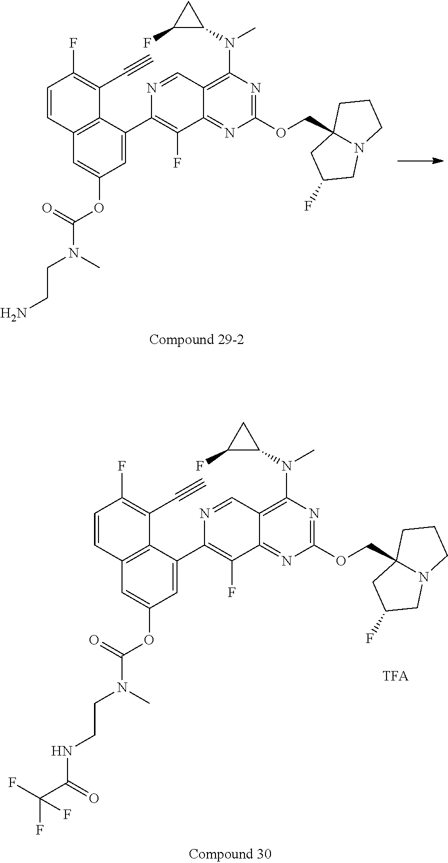 [SMILES: C#Cc1c(F)ccc2cc(OC(=O)N(C)CCN)cc(-c3ncc4c(N(C)[C@H]5C[C@@H]5F)nc(OC[C@@]56CCCN5C[C@H](F)C6)nc4c3F)c12 ; C#Cc1c(F)ccc2cc(OC(=O)N(C)CCNC(=O)C(F)(F)F)cc(-c3ncc4c(N(C)[C@H]5C[C@@H]5F)nc(OC[C@@]56CCCN5C[C@H](F)C6)nc4c3F)c12]

A solution of Compound 29-2 (260 mg, 0.38 mmol), trifluoroacetic anhydride (1 mL) and DIEA (2 mL) in DCM (5 mL) was stirred at room temperature for 2 hours. The reaction mixture was diluted with DCM (20 mL) and washed with water (20 mL) and sat·NH₄Cl (20 mL). The organic layer was dried over anhydrous Na₂SO₄ and concentrated under reduced pressure. The crude product was purified by Pre-HPLC with the following condition (Daisogel C18, 50 mm×250 mm, 10 um; A: 0.1% TFA in water, B: CH₃CN, Gradient: 15% B to 55% B in 50 min at a flow rate of 60 mL/min, 235 nm and freeze-dried to give Compound 30 (TFA salt, 70.5 mg, 20.66% yield). MS: m/z: 774[M+H]⁺.

### Example 31

### 1-(((2-((((5-ethynyl-6-fluoro-4-(8-fluoro-4-(((1S,2S)-2-fluorocyclopropyl)(methyl)amino)-2-(((2R,7aS)-2-fluorotetrahydro-1H-pyrrolizin-7a(5H)-yl)methoxy)pyrido[4,3-d]pyrimidin-7-yl)naphthalen-2-yl)oxy)carbonyl)(methyl)amino)ethyl)carbamoyl)oxy)ethyl isobutyrate 2,2,2-trifluoroacetic acid (“Compound 31 (TFA salt)”)

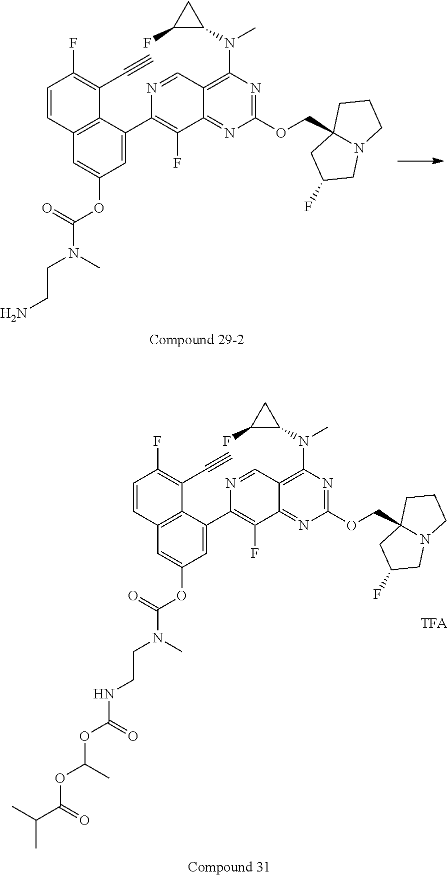 [SMILES: C#Cc1c(F)ccc2cc(OC(=O)N(C)CCN)cc(-c3ncc4c(N(C)[C@H]5C[C@@H]5F)nc(OC[C@@]56CCCN5C[C@H](F)C6)nc4c3F)c12 ; C#Cc1c(F)ccc2cc(OC(=O)N(C)CCNC(=O)OC(C)OC(=O)C(C)C)cc(-c3ncc4c(N(C)[C@H]5C[C@@H]5F)nc(OC[C@@]56CCCN5C[C@H](F)C6)nc4c3F)c12]

To a solution of Compound 29-2 (102 mg, 0.15 mmol), DIEA (1 mL) in DCM (5 mL) was added 1-(((4-nitrophenoxy)carbonyl)oxy)ethyl isobutyrate (39 mg, 0.13 mmol), then stirred at room temperature for 4 hours. The reaction mixture was diluted with water (20 mL) and extracted with DCM (20 mL). The organic layer was dried over anhydrous Na₂SO₄ and concentrated under reduced pressure. The crude product was purified by Pre-HPLC with the following condition (Agela Durashell C18, 30 mm×250 mm, 10 um; A: 0.1% TFA in water, B: CH₃CN, Gradient: 15% B to 49% B in 37 min at a flow rate of 40 mL/min, 240 nm) and freeze-dried to give Compound 31 (TFA salt, 57.1 mg, 39.96% yield). MS: m/z: 836[M+H]⁺.

### Example 32

### 5-ethynyl-6-fluoro-4-(8-fluoro-4-(((1S,2S)-2-fluorocyclopropyl)(methyl)amino)-2-((2-fluorotetrahydro-1H-pyrrolizin-7a(5H)-yl)methoxy-d2)pyrido[4,3-d]pyrimidin-7-yl)naphthalen-2-ol 2,2,2-trifluoroacetic acid (“Compound 32 (TFA salt)”)

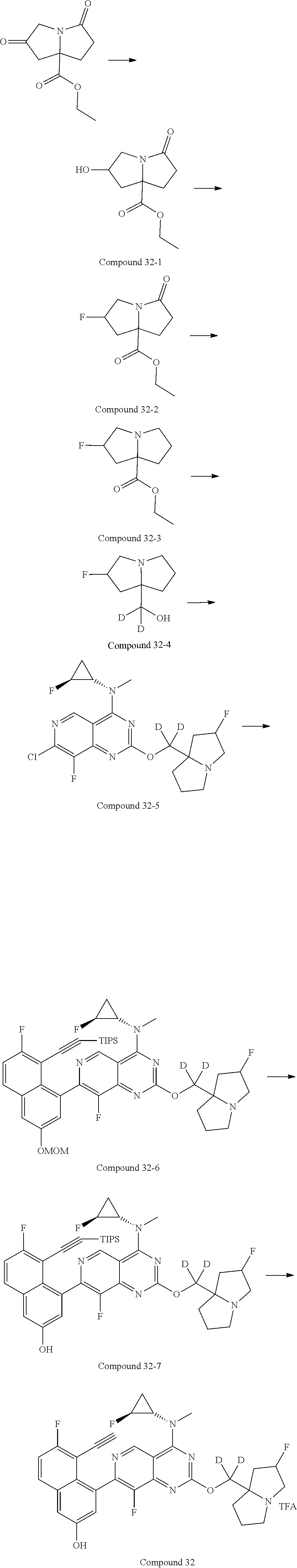

To a solution of ethyl 2,5-dioxotetrahydro-1H-pyrrolizine-7a(5H)-carboxylate (1.04 g, 4.92 mmol) in EtOH (6 mL) was slowly added NaBH₄ (72 mg, 1.90 mmol) at 0° C. under nitrogen atmosphere. The mixture was stirred at 0° C. for 1 hour. The reaction mixture was quenched with sat. NH₄Cl (2 mL) and the mixture was concentrated under reduced pressure. The residue was purified by silica gel chromatography (eluted with DCM:MeOH=50:1 to 20:1, v/v) to give Compound 32-1 (851 mg, 81.05% yield). MS: m/z: 214 [M+H]⁺.

To a solution of Compound 32-1 (851 mg, 3.99 mmol) in DCM (10 mL) was added a solution of DAST (970 mg, 6.02 mmol) drop-wise at −70° C. under nitrogen atmosphere. The reaction mixture was allowed to warm to room temperature and stirred for 3 hours. The mixture was quenched with MeOH (2 mL), then diluted with water (10 mL) and extracted with DCM (20 mL×3). The combined organic layers were washed with brine (30 mL, dried over anhydrous Na₂SO₄ and concentrated under reduced pressure. The residue was purified by silica gel chromatography (eluted with Hex:EtOAc=10:1 to 1:1, v/v) to give Compound 32-2 (401 mg, 46.68% yield). MS: m/z: 216 [M+H]⁺.

To a solution of Compound 32-2 (383 mg, 1.78 mmol) in THF (8 mL) was slowly added BH₃ (3.5 mL, 1M in THF) at 0° C. under nitrogen atmosphere. The mixture was stirred at room temperature for 8 hours. The reaction was quenched with MeOH (2 mL) and concentrated under reduced pressure. The residue was dissolved in MeOH (4 mL) and stirred at reflux temperature for 1 hour. The mixture was concentrated under reduced pressure and purified by silica gel chromatography (eluted with Hex:EtOAc=100:1 to 1:1, v/v) to give Compound 32-3 (264 mg, 1.31 mmol, 73.72% yield). MS: m/z: 202 [M+H]⁺.

To a solution of Compound 32-3 (245 mg, 1.22 mmol) in THF (5 mL) was slowly added LiAlD₄ (62 mg, 1.24 mmol) at 0° C. under nitrogen atmosphere. The mixture was stirred at room temperature for 2 hours. The reaction mixture was quenched with water (0.1 mL), NaOH (15%, 0.1 mL) and water (0.1 mL). The mixture was filtered and the filtrate was concentrated under reduced pressure. The residue was purified by silica gel chromatography (eluted with DCM:MeOH=10:1 to 2:1, v/v) to give Compound 32-4 (174 mg, 88.65% yield). MS: m/z: 162 [M+H]⁺.

To a solution of Compound 3A-4 (185 mg, 0.606 mmol), Compound 32-4 (97 mg, 0.602 mmol) in THF (5 mL) was added t-BuONa (73 mg, 0.760 mmol) in portions at −10° C. under nitrogen atmosphere. The reaction mixture was stirred for 3 hours. The mixture was allowed to warm to room temperature and extracted with EtOAc (30 mL). The organic layer was washed with brine (2×30 mL), dried over anhydrous Na₂SO₄ and concentrated under reduced pressure. The residue was purified by Pre-TLC (eluted with DCM:MeOH=15:1, v/v) to give Compound 32-5 (127 mg, 48.73% yield). MS: m/z: 430 [M+H]⁺.

To a solution of Compound 32-5 (127 mg, 0.295 mmol), INT3 (203 mg, 0.396 mmol) in 1,4-dioxane (5 mL) and water (1 mL) were added Cs₂CO₃ (275 mg, 0.844 mmol) and cataCxium A Pd G₃ (25 mg, 0.034 mmol). The reaction mixture was stirred at 100° C. for overnight under nitrogen atmosphere. The mixture was filtered and the filtrate was concentrated under reduced pressure. The residue was purified by Pre-TLC (eluted with DCM:MeOH=15:1, v/v) to give Compound 32-6 (173 mg, 0.222 mmol, 75.07% yield). MS: m/z: 780 [M+H]⁺.

A solution of Compound 32-6 (173 mg, 0.222 mmol) and HCl (0.8 mL, 4M in dioxane) in acetonitrile (3 mL) was stirred at room temperature for 2 hours. The solution was diluted with sat. aq. NaHCO₃ (20 mL), extracted with EtOAc (30 mL). The organic layer was washed with brine (30 mL), dried over anhydrous Na₂SO₄ and concentrated under reduced pressure to give Compound 32-7 (crude, 164 mg, 100.47% yield). MS: m/z: 736 [M+H]⁺.

To a solution of Compound 32-7 (164 mg, 0.223 mmol) in DMF (3 mL) was added CsF (0.35 g, 2.30 mmol). The reaction was stirred at 44° C. for 2 hours under nitrogen atmosphere. The mixture was diluted with water (10 mL) and extracted with EtOAc (20 mL). The organic layer was dried over anhydrous Na₂SO₄ and concentrated under reduced pressure. The residue was purified by Prep-HPLC (Daisogel C18, 50 mm×250 mm, 10 urn; A: 0.10% TFA in water, B: CH₃CN, Gradient: 15% B to 48% B in 45 min at a flow rate of 60 mL/min, 235 nm) to freeze-dried to afford Compound 32 (TFA salt, 97.0 mg, 0.140 mmol, 62.75% yield). MS: m/z: 580 [M+H]⁺.

The following compounds in the Table 16 were synthesized using the above procedures or modification procedures:

TABLE 16

| Example | Compound | Structure | IUNPAC Name |
|---|---|---|---|
| 33 | Compound 33 (TFA salt) | 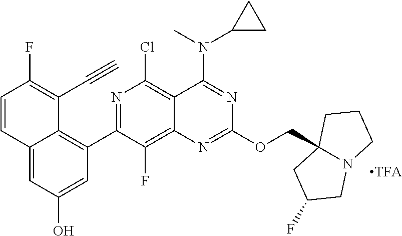 [SMILES: C#Cc1c(F)ccc2cc(O)cc(-c3nc(Cl)c4c(N(C)C5CC5)nc(OC[C@@]56CCCN5C[C@H](F)C6)nc4c3F)c12] | 4-(5-chloro-4-(cyclopropyl(methyl)amino)- 8-fluoro-2-(((2R,7aS)-2-fluorotetrahydro- 1H-pyrrolizin-7a(5H)-yl)methoxy)pyridin [4,3-d]pyrimidin-7-yl)-5-ethynyl-6-fluoro- naphthalen-2-ol 2,2,2-trifluoroacetic acid |
| 34 | Compound 34 | 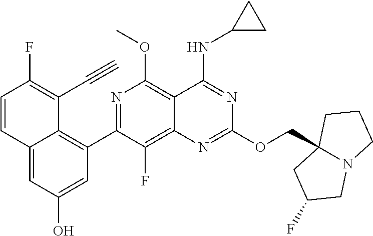 [SMILES: C#Cc1c(F)ccc2cc(O)cc(-c3nc(OC)c4c(NC5CC5)nc(OC[C@@]56CCCN5C[C@H](F)C6)nc4c3F)c12] | 4-(4-(cyclopropylamino)-8-fluoro-2-(((2R, 7aS)-2-fluorotetrahydro-1H-pyrrolizin-7a(5H)- yl)methoxy)-5-methoxypyrido[4,3-d]pyrimidin- 7-yl)-5-ethynyl-6-fluoronaphthalen-2-ol |
| 35 | Compound 35 | 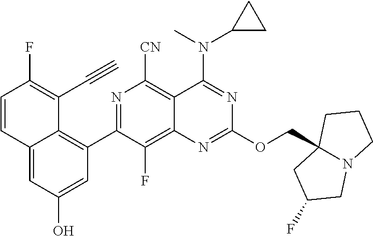 [SMILES: C#Cc1c(F)ccc2cc(O)cc(-c3nc(C#N)c4c(N(C)C5CC5)nc(OC[C@@]56CCCN5C[C@H](F)C6)nc4c3F)c12] | 4-(cyclopropyl(methyl)amino)-7-(8-ethynyl- 7-fluoro-3-hydroxynaphthalen-1-yl)-8- fluoro-2-(((2R,7aS)-2-fluorotetrahydro-1H- pyrrolizin-7a(5H)-yl)methoxy)pyrido[4,3-d] pyrimidine-5-carbonitrile |
| 36 | Compound 36 (TFA salt) | 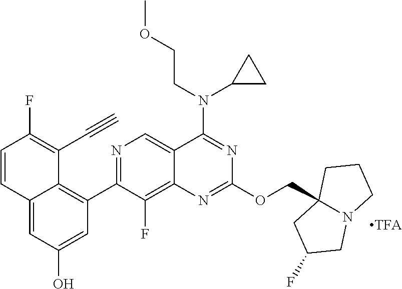 [SMILES: C#Cc1c(F)ccc2cc(O)cc(-c3ncc4c(N(CCOC)C5CC5)nc(OC[C@@]56CCCN5C[C@H](F)C6)nc4c3F)c12] | 4-(4-(cyclopropyl(2-methoxyethyl)amino)- 8-fluoro-2-(((2R,7aS)-2-fluorotetrahydro- 1H-pyrrolizin-7a(5H)-yl)methoxy)pyrido [4,3-d]pyrimidin-7-yl)-5-ethynyl-6-fluoro naphthalen-2-ol 2,2,2-trifluoroacetic acid |
| 37 | Compound 37 (TFA salt) | 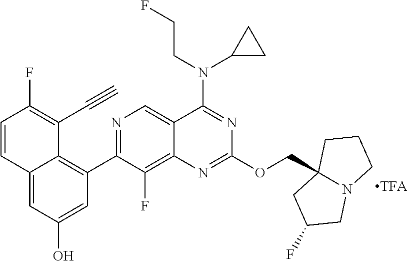 [SMILES: C#Cc1c(F)ccc2cc(O)cc(-c3ncc4c(N(CCF)C5CC5)nc(OC[C@@]56CCCN5C[C@H](F)C6)nc4c3F)c12] | 4-(4-(cyclopropyl(2-fluoroethyl)amino)-8- fluoro-2-(((2R,7aS)-2-fluorotetrahydro-1H- pyrrolizin-7a(5H)-yl)methoxy)pyrido [4,3-d]pyrimidin-7-yl)-5-ethynyl-6-fluoro- naphthalen-2-ol 2,2,2-trifluoroacetic acid |
| 38 | Compound 38 (TFA salt) | 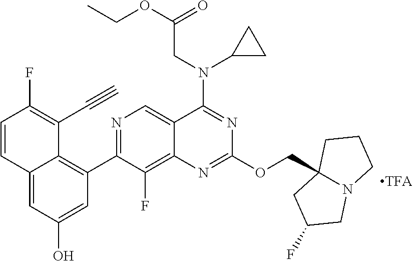 [SMILES: C#Cc1c(F)ccc2cc(O)cc(-c3ncc4c(N(CC(=O)OCC)C5CC5)nc(OC[C@@]56CCCN5C[C@H](F)C6)nc4c3F)c12] | Ethyl N-cyclopropyl-N-(7-(8-ethynyl-7-fluoro-3- hydroxynaphthalen-1-yl)-8-fluoro-2-(((2R, 7aS)-2-fluorotetrahydro-1H-pyrrolizin-7a(5H)- yl)methoxy)pyrido[4,3-d]pyrimidin- 4-yl)glycinate 2,2,2-trifluoroacetic acid |
| 39 | Compound 39 (TFA salt) | 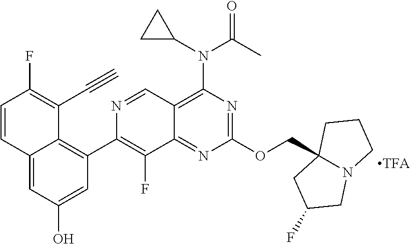 [SMILES: C#Cc1c(F)ccc2cc(O)cc(-c3ncc4c(N(C(C)=O)C5CC5)nc(OC[C@@]56CCCN5C[C@H](F)C6)nc4c3F)c12] | N-cyclopropyl-N-(7-(8-ethynyl-7-fluoro-3- hydroxynaphthalen-1-yl)-8-fluoro-2-(((2R, 7aS)-2-fluorotetrahydro-1H-pyrrolizin- 7a(5H)-yl)methoxy)pyrido[4,3-d]pyrimidin- 4-yl)acetamide 2,2,2-trifluoroacetic acid |
| 40 | Compound 40 (TFA salt) | 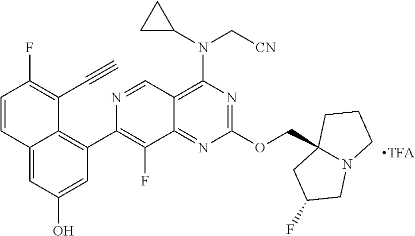 [SMILES: C#Cc1c(F)ccc2cc(O)cc(-c3ncc4c(N(CC#N)C5CC5)nc(OC[C@@]56CCCN5C[C@H](F)C6)nc4c3F)c12] | 2-(cyclopropyl(7-(8-ethynyl-7-fluoro-3- hydroxynaphthalen-1-yl)-8-fluoro-2-(((2R, 7aS)-2-fluorotetrahydro-1H-pyrrolizin-7a(5H)- yl)methoxy)pyrido[4,3-d]pyrimidin-4-yl) amino)acetonitrile 2,2,2-trifluoroacetic acid |
| 41 | Compound 41 | 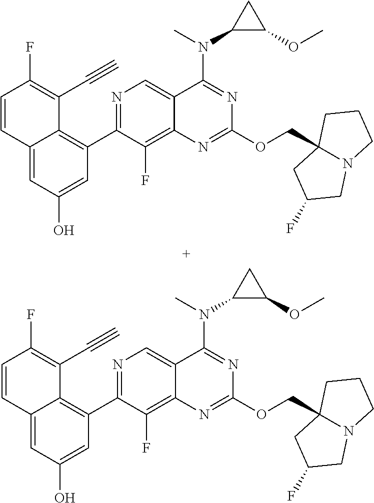 [SMILES: C#Cc1c(F)ccc2cc(O)cc(-c3ncc4c(N(C)[C@H]5C[C@@H]5OC)nc(OC[C@@]56CCCN5C[C@H](F)C6)nc4c3F)c12 ; C#Cc1c(F)ccc2cc(O)cc(-c3ncc4c(N(C)[C@@H]5C[C@H]5OC)nc(OC[C@@]56CCCN5C[C@H](F)C6)nc4c3F)c12] | 5-ethynyl-6-fluoro-4-(8-fluoro-2-(((2R,7aS)- 2-fluorotetrahydro-1H-pyrrolizin-7a(5H)- yl)methoxy)-4-((trans-2-methoxycyclo- propyl)(methyl)amino)pyrido[4,3-d] pyrimidin-7-yl)naphthalen-2-ol |
| 42 | Compound 42 (TFA salt) | 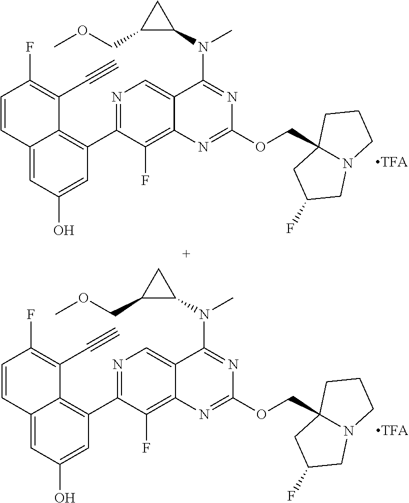 [SMILES: C#Cc1c(F)ccc2cc(O)cc(-c3ncc4c(N(C)[C@@H]5C[C@H]5COC)nc(OC[C@@]56CCCN5C[C@H](F)C6)nc4c3F)c12 ; C#Cc1c(F)ccc2cc(O)cc(-c3ncc4c(N(C)[C@H]5C[C@@H]5COC)nc(OC[C@@]56CCCN5C[C@H](F)C6)nc4c3F)c12] | 5-ethynyl-6-fluoro-4-(8-fluoro-2-(((2R,7aS)- 2-fluorotetrahydro-1H-pyrrolizin-7a(5H)- yl)methoxy)-4-((trans-2-(methoxymethyl) cyclopropyl)(methyl)amino)pyrido[4,3-d] pyrimidin-7-yl)naphthalen-2-ol 2,2,2-trifluoroacetic acid |
| 43 | Compound 43 (TFA salt) | 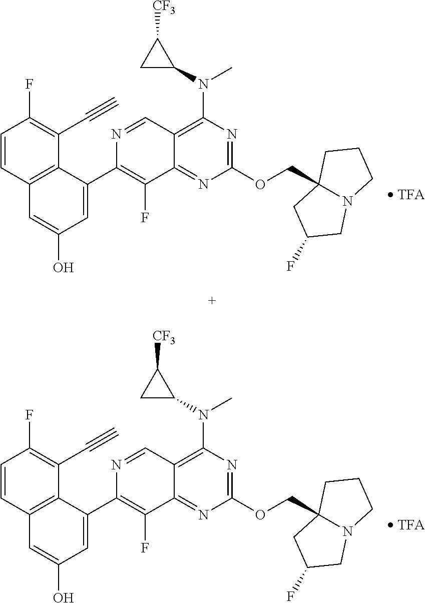 [SMILES: C#Cc1c(F)ccc2cc(O)cc(-c3ncc4c(N(C)[C@H]5C[C@@H]5C(F)(F)F)nc(OC[C@@]56CCCN5C[C@H](F)C6)nc4c3F)c12 ; C#Cc1c(F)ccc2cc(O)cc(-c3ncc4c(N(C)[C@@H]5C[C@H]5C(F)(F)F)nc(OC[C@@]56CCCN5C[C@H](F)C6)nc4c3F)c12] | 5-ethynyl-6-fluoro-4-(8-fluoro-2-(((2R,7aS)- 2-fluorotetrahydro-1H-pyrrolizin-7a(5H)- yl)methoxy)-4-(methyl(trans-2-(trifluoro- methyl)cyclopropyl)amino)pyrido[4,3-d] pyrimidin-7-yl)naphthalen-2-ol 2,2,2-trifluoroacetic acid |
| 44 | Compound 44 (TFA salt) | 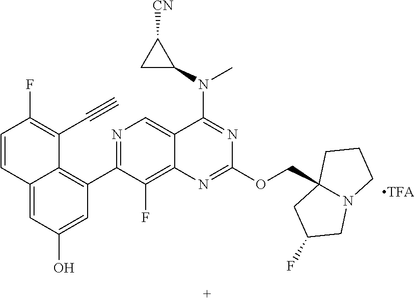 [SMILES: C#Cc1c(F)ccc2cc(O)cc(-c3ncc4c(N(C)[C@H]5C[C@@H]5C#N)nc(OC[C@@]56CCCN5C[C@H](F)C6)nc4c3F)c12] | trans-2-((7-(8-ethynyl-7-fluoro-3-hydroxy naphthalen-1-yl)-8-fluoro-2-(((2R,7aS)-2- fluorotetrahydro-1H-pyrrolizin-7a(5H)- yl)methoxy)pyrido[4,3-d]pyrimidin-4-yl) (methyl)amino)cyclopropane-1-carbonitrile 2,2,2-trifluoroacetic acid |
| | | 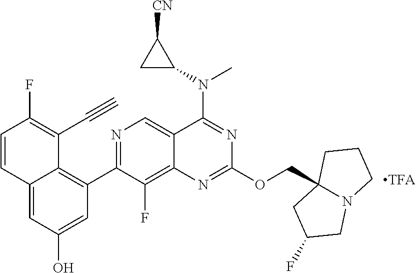 [SMILES: C#Cc1c(F)ccc2cc(O)cc(-c3ncc4c(N(C)[C@@H]5C[C@H]5C#N)nc(OC[C@@]56CCCN5C[C@H](F)C6)nc4c3F)c12] | |
| 45 | Compound 45 | 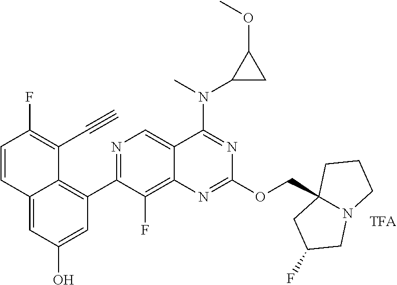 [SMILES: C#Cc1c(F)ccc2cc(O)cc(-c3ncc4c(N(C)C5CC5OC)nc(OC[C@@]56CCCN5C[C@H](F)C6)nc4c3F)c12] | 5-ethynyl-6-fluoro-4-(8-fluoro-2-(((2R,7aS)- 2-fluorotetrahydro-1H-pyrrolizin-7a(5H)- yl)methoxy)-4-((2-methoxycyclopropyl) (methyl)amino)pyrido[4,3-d]pyrimidin- 7-yl)naphthalen-2-ol 2,2,2-trifluoroacetic acid |
| 46 | Compound 46 | 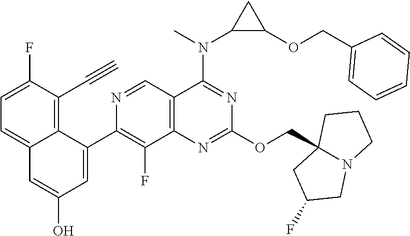 [SMILES: C#Cc1c(F)ccc2cc(O)cc(-c3ncc4c(N(C)C5CC5OCc5ccccc5)nc(OC[C@@]56CCCN5C[C@H](F)C6)nc4c3F)c12] | 4-(4-((2-(benzyloxy)cyclopropyl)(methyl) amino)-8-fluoro-2-(((2R,7aS)-2-fluoro- tetrahydro-1H-pyrrolizin-7a(5H)-yl)methoxy) pyrido[4,3-d]pyrimidin-7-yl)-5-ethynyl-6- fluoronaphthalen-2-ol |
| 47 | Compound 47 | 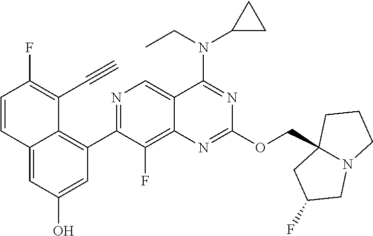 [SMILES: C#Cc1c(F)ccc2cc(O)cc(-c3ncc4c(N(CC)C5CC5)nc(OC[C@@]56CCCN5C[C@H](F)C6)nc4c3F)c12] | 4-(4-(cyclopropyl(ethyl)amino)-8-fluoro-2- (((2R,7aS)-2-fluorotetrahydro-1H-pyrrolizin- 7a(5H)-yl)methoxy)pyrido[4,3-d]pyrimidin- 7-yl)-5-ethynyl-6-fluoronaphthalen-2-ol |
| 48 | Compound 48 | 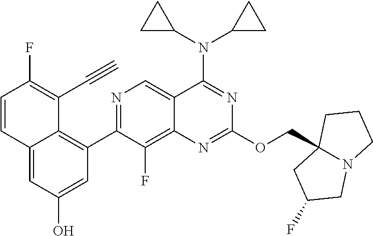 [SMILES: C#Cc1c(F)ccc2cc(O)cc(-c3ncc4c(N(C5CC5)C5CC5)nc(OC[C@@]56CCCN5C[C@H](F)C6)nc4c3F)c12] | 4-(4-(dicyclopropylamino)-8-fluoro-2-(((2R, 7aS)-2-fluorotetrahydro-1H-pyrrolizin-7a(5H)- yl)methoxy)pyrido[4,3-d]pyrimidin-7-yl)-5- ethynyl-6-fluoronaphthalen-2-ol |
| 49 | Compound 49 | 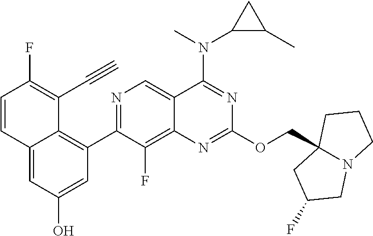 [SMILES: C#Cc1c(F)ccc2cc(O)cc(-c3ncc4c(N(C)C5CC5C)nc(OC[C@@]56CCCN5C[C@H](F)C6)nc4c3F)c12] | 5-ethynyl-6-fluoro-4-(8-fluoro-2-(((2R,7aS)- 2-fluorotetrahydro-1H-pyrrolizin-7a(5H)- yl)methoxy)-4-(methyl(2-methylcyclo- propyl)amino)pyrido[4,3-d]pyrimidin-7- yl)naphthalen-2-ol |
| 50 | Compound 50 | 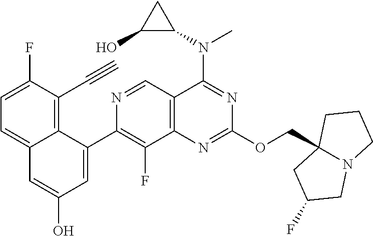 [SMILES: C#Cc1c(F)ccc2cc(O)cc(-c3ncc4c(N(C)[C@H]5C[C@@H]5O)nc(OC[C@@]56CCCN5C[C@H](F)C6)nc4c3F)c12] | 5-ethynyl-6-fluoro-4-(8-fluoro-2-(((2R,7aS)- 2-fluorotetrahydro-1H-pyrrolizin-7a(5H)- yl)methoxy)-4-(((1S,2S)-2-hydroxy- cyclopropyl)(methyl)amino)pyrido[4,3-d] pyrimidin-7-yl)naphthalen-2-ol |
| 51 | Compound 51 | 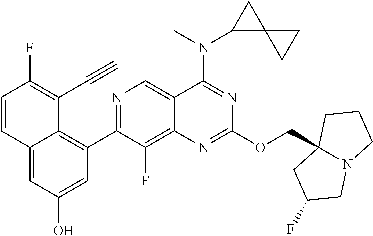 [SMILES: C#Cc1c(F)ccc2cc(O)cc(-c3ncc4c(N(C)C5CC56CC6)nc(OC[C@@]56CCCN5C[C@H](F)C6)nc4c3F)c12] | 5-ethynyl-6-fluoro-4-(8-fluoro-2-(((2R,7aS)- 2-fluorotetrahydro-1H-pyrrolizin-7a(5H)- yl)methoxy)-4-(methyl(spiro[2.2]pentan-1- yl)amino)pyrido[4,3-d]pyrimidin-7- yl)naphthalen-2-ol |
| 52 | Compound 52 | 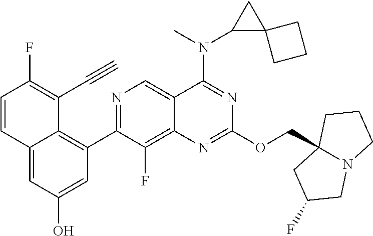 [SMILES: C#Cc1c(F)ccc2cc(O)cc(-c3ncc4c(N(C)C5CC56CCC6)nc(OC[C@@]56CCCN5C[C@H](F)C6)nc4c3F)c12] | 5-ethynyl-6-fluoro-4-(8-fluoro-2-(((2R,7aS)- 2-fluorotetrahydro-1H-pyrrolizin-7a(5H)- yl)methoxy)-4-(methyl(spiro[2.3]hexan- 1-yl)amino)pyrido[4,3-d]pyrimidin-7- yl)naphthalen-2-ol |
| 53 | Compound 53 | 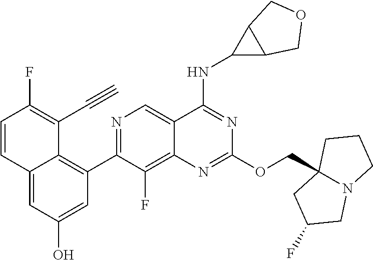 [SMILES: C#Cc1c(F)ccc2cc(O)cc(-c3ncc4c(NC5C6COCC65)nc(OC[C@@]56CCCN5C[C@H](F)C6)nc4c3F)c12] | 4-(4-((3-oxabicyclo[3.1.0]hexan-6-yl)amino)- 8-fluoro-2-(((2R,7aS)-2-fluorotetrahydro- 1H-pyrrolizin-7a(5H)-yl)methoxy) pyrido[4,3-d]pyrimidin-7-yl)-5-ethynyl-6- fluoronaphthalen-2-ol |
| 54 | Compound 54 | 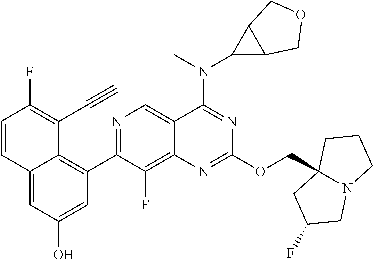 [SMILES: C#Cc1c(F)ccc2cc(O)cc(-c3ncc4c(N(C)C5C6COCC65)nc(OC[C@@]56CCCN5C[C@H](F)C6)nc4c3F)c12] | 4-(4-((3-oxabicyclo[3.1.0]hexan-6-yl)(methyl) amino)-8-fluoro-2-(((2R,7aS)-2-fluoro tetrahydro-1H-pyrrolizin-7a(5H)-yl) methoxy)pyrido[4,3-d]pyrimidin-7-yl)-5- ethynyl-6-fluoronaphthalen-2-ol |
| 55 | Compound 55 | 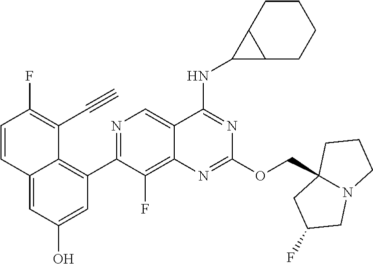 [SMILES: C#Cc1c(F)ccc2cc(O)cc(-c3ncc4c(NC5C6CCCCC65)nc(OC[C@@]56CCCN5C[C@H](F)C6)nc4c3F)c12] | 4-(4-(bicyclo[4.1.0]heptan-7-ylamino)-8- fluoro-2-(((2R,7aS)-2-fluorotetrahydro-1H- pyrrolizin-7a(5H)-yl)methoxy)pyrido[4,3-d] pyrimidin-7-yl)-5-ethynyl-6-fluoronaphthalen- 2-ol |
| 56 | Compound 56 | 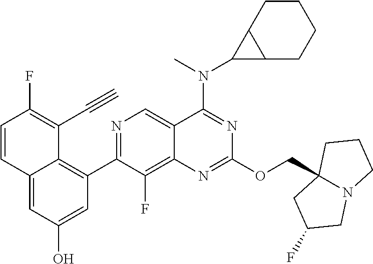 [SMILES: C#Cc1c(F)ccc2cc(O)cc(-c3ncc4c(N(C)C5C6CCCCC65)nc(OC[C@@]56CCCN5C[C@H](F)C6)nc4c3F)c12] | 4-(4-(bicyclo[4.1.0]heptan-7-yl(methyl) amino)-8-fluoro-2-(((2R,7aS)-2-fluoro- tetrahydro-1H-pyrrolizin-7a(5H)-yl)methoxy) pyrido[4,3-d]pyrimidin-7-yl)-5-ethynyl-6- fluoronaphthalen-2-ol |
| 57 | Compound 57 | 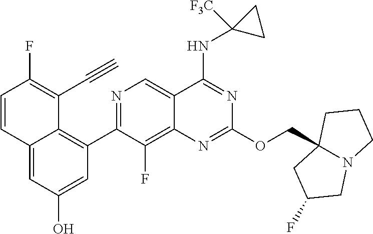 [SMILES: C#Cc1c(F)ccc2cc(O)cc(-c3ncc4c(NC5(C(F)(F)F)CC5)nc(OC[C@@]56CCCN5C[C@H](F)C6)nc4c3F)c12] | 5-ethynyl-6-fluoro-4-(8-fluoro-2-(((2R,7aS)- 2-fluorotetrahydro-1H-pyrrolizin-7a(5H)- yl)methoxy)-4-((1-(trifluoromethyl) cyclopropyl)amino)pyrido[4,3-d]pyrimidin- 7-yl)naphthalen-2-ol |
| 58 | Compound 58 | 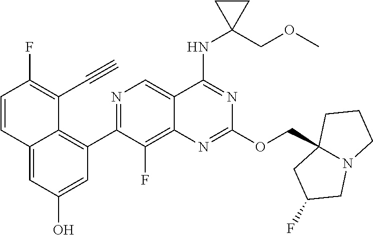 [SMILES: C#Cc1c(F)ccc2cc(O)cc(-c3ncc4c(NC5(COC)CC5)nc(OC[C@@]56CCCN5C[C@H](F)C6)nc4c3F)c12] | 5-ethynyl-6-fluoro-4-(8-fluoro-2-(((2R,7aS)- 2-fluorotetrahydro-1H-pyrrolizin-7a(5H)- yl)methoxy)-4-((1-(methoxymethyl) cyclopropyl)amino)pyrido[4,3-d]pyrimidin- 7-yl)naphthalen-2-ol |
| 59 | Compound 59 | 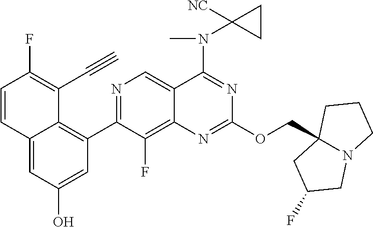 [SMILES: C#Cc1c(F)ccc2cc(O)cc(-c3ncc4c(N(C)C5(C#N)CC5)nc(OC[C@@]56CCCN5C[C@H](F)C6)nc4c3F)c12] | 1-((7-(8-ethynyl-7-fluoro-3-hydroxy- naphthalen-1-yl)-8-fluoro-2-(((2R,7aS)-2- fluorotetrahydro-1H-pyrrolizin-7a(5H)-yl) methoxy)pyrido[4,3-d]pyrimidin-4-yl)(methyl) amino)cyclopropane-1-carbonitrile |

### Pharmacological Experiments

### 1. SOS1 Catalyzed Nucleotide Exchange Assay

The inhibition activity of each of compounds on GDP form K-Ras was evaluated by SOS1 catalyzed nucleotide exchange assays. K-Ras G12D, K-Ras G12V, K-Ras G12C, K-Ras G13D, K-Ras G12A, K-Ras G12R, K-Ras Q61H and K-Ras WI proteins were used in this assay.

Briefly, K-Ras (His tag, aa 1-169) pre-loaded with GDP was pre-incubated with each of compounds in the presence of 10 nM GDP in a 384-well plate (Greiner) for 15-60 mins, then purified SOS1 Ext (Flag tag, aa 564-1049), BODIPY™ FL GTP (Invitrogen) and monoclonal antibody anti 6HIS—Tb cryptate Gold (Cisbio) were added to the assay wells and incubated for 4 hours at 25° C. (Specially, we did not add SOS1 in the K-Ras G13D assay). Wells containing the same percent of DMSO served as vehicle control, and wells without K-Ras served as low control. TR-FRET signals were read on Tecan Spark multimode microplate reader. The parameters were F486: Excitation 340 nm, Emission 486 nm, Lag time 100 pas, Integration time 200 pas; F515: Excitation 340 nm, Emission 515 nm, Lag time 100 pas, Integration time 200 pas. TR-FRET ratios for each individual wells were calculated by equation: TR-FRET ratio=(Signal F515/Signal F486)*10000. The percent of activation of compounds treated wells were normalized between vehicle control and low control (00 Activation=(TR-FRET ratio_{Compound treated}−TR-FRET ratio_{Low control})/(TR-FRET ratio_{Vehicle control}−TR-FRET ratio_{Low control})*1000%). Then the data were analyzed either by filling a 4-parameter logistic model or by Excel to calculate IC₅₀ values. The results are shown in the following Table 17.

2. GTP-K-Ras and cRAF Interaction Assay

The inhibition activity of each of compounds on GTP form K-Ras was evaluated by GppNp-K-Ras and cRAF interaction assays. GppNp is an analog of GTP. K-Ras G12D, K-Ras G12V, K-Ras G12C, K-Ras G13D, K-Ras G12A, K-Ras G12R, K-Ras Q61H and K-Ras WI proteins were used in this assay.

Briefly, K-Ras (His tag, aa 1-169) pre-loaded with GppNp was pre-incubated with each of compounds in the presence of 200 μM GTP in a 384-well plate (Greiner) for 15-60 mins, then cRAF RBD (GST tag, aa 50-132, CreativeBioMart), monoclonal antibody anti GST-d2 (Cisbio) and monoclonal antibody anti 6HIS—Tb cryptate Gold (Cisbio) were added to the assay wells and incubated for 2 hours at 25° C. Wells containing same percent of DMSU served as vehicle control, and wells without K-Ras served as low control. HTRP signals were read on Tecan Spark multimode microplate reader and HTRF ratios were calculated under manufacturer's instructions.

The percent of activation of compounds treated wells were normalized between vehicle control and low control (% Activation=(HTRF ratio_{Compound treated}−HTRF ratio_{Low control})/(HTRF ratio_{Vehicle control}−HTRF ratio_{Low control})*100%). Then the data were analyzed either by fitting a 4-parameter logistic model or by Excel to calculate IC₅₀ values. The results are shown in the following Table 17.

TABLE 17

| | Biochemical assays, IC₅₀ (nM) | | | | | | | |
|---|---|---|---|---|---|---|---|---|
| Com- | K-Ras G12D | | K-Ras G12V | | K-Ras G12C | | K-Ras G13D | |
| pound | GDP | GppNp | GDP | GppNp | GDP | GppNp | GDP | GppNp |
| Com- | 3.90 | 50.2 | 0.683 | 204 | 4.21 | 426 | 15.2 | 10.1 |
| pound 1 | | | | | | | | |
| Com- | 4.07 | 122 | 2.17 | 232 | | | | |
| pound 2 | | | | | | | | |
| Com- | 2.64 | 28.3 | 2.77 | 77.9 | 3.21 | 283 | 4.98 | 6.97 |
| pound 3 | | | | | | | | |
| Com- | 1.21 | 22.2 | 1.97 | 54.8 | 0.526 | 122 | 0.939 | 2.77 |
| pound 3A | | | | | | | | |
| Com- | 13.3 | 206 | 12.2 | 770 | | | | |
| pound 3B | | | | | | | | |
| Com | 3.41 | 88.8 | 0.949 | 234 | 3.83 | 587 | 10.1 | 13.7 |
| pound 4 | | | | | | | | |
| Com- | 2.84 | 65.2 | 3.23 | 145 | 2.48 | 317 | 3.29 | 6.29 |
| pound 5 | | | | | | | | |
| Com- | 1.32 | 118 | 4.43 | 167 | 3.03 | 564 | 4.38 | 8.55 |
| pound 6 | | | | | | | | |
| Com- | 0.701 | 17.6 | 1.46 | 46.6 | | | | |
| pound 7 | | | | | | | | |
| Com- | 46.9 | 801 | | >1000 | | | | |
| pound 8 | | | | | | | | |
| Com- | 2.00 | 66.7 | 4.42 | 161 | | | | |
| pound 9 | | | | | | | | |
| Com- | 24.20 | 519 | 5.98 | 736 | | | | |
| pound 10 | | | | | | | | |
| Com- | 12.5 | 252 | 8.13 | 307 | | | | |
| pound 11 | | | | | | | | |
| Com- | 32.9 | 460 | 7.39 | 1127 | | | | |
| pound 12 | | | | | | | | |
| Com- | 30.3 | 1308 | 5.11 | 1823 | | | | |
| pound 13 | | | | | | | | |
| Com- | 18.0 | 607.00 | 8.85 | >1000 | | | | |
| pound 14 | | | | | | | | |
| Com- | 3.16 | 172 | 6.53 | 568 | 3.88 | >1000 | | |
| pound 15 | | | | | | | | |
| Com- | 1.98 | 90.5 | 3.81 | 441 | 5.21 | 731 | 6.97 | 10.4 |
| pound 16 | | | | | | | | |
| Com- | 8.94 | 574 | 8.24 | 984 | | | | |
| pound 17 | | | | | | | | |
| Com- | 16.3 | 637 | 2.27 | 1587 | | | 13.9 | 48.3 |
| pound 18 | | | | | | | | |
| Com- | 27.3 | 1477 | 9.60 | 3653 | | | >100 | >100 |
| pound 19 | | | | | | | | |
| Com- | 12.8 | 730 | 2.96 | 1302 | | | | |
| pound 22 | | | | | | | | |
| Com- | 8.59 | 320 | 2.97 | >1000 | | | | |
| pound 25 | | | | | | | | |
| Com- | 6.92 | 126 | 1.89 | 172 | | | 10.4 | 20.3 |
| pound 26 | | | | | | | | |
| Com- | 6.07 | 657 | 6.17 | >1000 | | | | |
| pound 33 | | | | | | | | |
| Com- | 76.2 | 704 | 24.0 | >1000 | | | | |
| pound 34 | | | | | | | | |
| Com- | 380 | >1000 | 117 | >1000 | | | | |
| pound 35 | | | | | | | | |
| Com- | 61.7 | >1000 | 18.4 | >1000 | | | | |
| pound 36 | | | | | | | | |
| Com- | 77.7 | >1000 | 24.5 | >1000 | | | | |
| pound 37 | | | | | | | | |
| Com- | 222 | >1000 | 121 | >1000 | | | | |
| pound 38 | | | | | | | | |
| Com- | 282 | >1000 | 25.9 | >1000 | | | | |
| pound 41 | | | | | | | | |
| Com- | 1944 | >1000 | 34.7 | >1000 | | | | |
| pound 42 | | | | | | | | |
| Com- | 75.9 | >1000 | 20.2 | >1000 | | | | |
| pound 43 | | | | | | | | |
| Com- | 16.3 | >1000 | 13.9 | >1000 | | | | |
| pound 44 | | | | | | | | |
| Com- | 1.72 | 156 | 1.13 | 292 | 4.69 | 872 | 5.07 | 13.1 |
| pound 47 | | | | | | | | |
| Com- | 134 | 2616 | 10.2 | 3706 | | | 50.0 | >100 |
| pound 48 | | | | | | | | |
| Com- | 1.99 | 222 | 1.11 | 333 | 6.01 | ~1000 | 9.24 | 20.4 |
| pound 49 | | | | | | | | |
| Com- | 61.7 | 744 | 16.2 | >1000 | | | | |
| pound 51 | | | | | | | | |
| Com- | 208 | >1000 | 76.0 | >1000 | | | | |
| pound 52 | | | | | | | | |
| Com- | 17.1 | 678 | 5.39 | 1364 | | | | |
| pound 53 | | | | | | | | |
| Com- | 774.1 | 1531 | 16.3 | 3719 | | | | |
| pound 55 | | | | | | | | |
| Com- | 157 | >1000 | 15.1 | >1000 | | | | |
| pound 57 | | | | | | | | |
| Com- | 25.9 | >1000 | 3.55 | >1000 | | | | |
| pound 58 | | | | | | | | |
| Com- | 62.4 | 3193 | 36.7 | 4466 | | | 200 | >100 |
| pound 59 | | | | | | | | |
| | Biochemical assays, IC₅₀ (nM) | | | | | | | |
| Com- | K-Ras WT | | K-Ras G12A | | K-Ras G12R | | K-Ras Q61H | |
| pound | GDP | GppNp | GDP | GppNp | GDP | GppNp | GDP | GppNp |
| Com- | 3.23 | 108 | 5.60 | 421 | 2.93 | 86.4 | 3.93 | 144 |
| pound 1 | | | | | | | | |
| Com- | 8.69 | | | | | | | |
| pound 2 | | | | | | | | |
| Com- | 2.40 | 112 | 3.83 | 272 | 3.25 | 30.3 | 2.42 | 45.3 |
| pound 3 | | | | | | | | |
| Com- | 0.707 | 77.9 | 0.999 | 149 | 1.04 | 42.9 | 0.729 | 70.0 |
| pound 3A | | | | | | | | |
| Com- | | | | | | | | |
| pound 3B | | | | | | | | |
| Com | 8.89 | 179 | 6.97 | 308 | 5.37 | 97.7 | 4.46 | 117 |
| pound 4 | | | | | | | | |
| Com- | 3.41 | 144 | 7.27 | 645 | 3.39 | 90.3 | 1.62 | 137 |
| pound 5 | | | | | | | | |
| Com- | 4.24 | 218 | 6.09 | >1000 | 3.11 | 101 | 2.87 | 388 |
| pound 6 | | | | | | | | |
| Com- | | | | | | | | |
| pound 7 | | | | | | | | |
| Com- | | | | | | | | |
| pound 8 | | | | | | | | |
| Com- | | | | | | | | |
| pound 9 | | | | | | | | |
| Com- | 29.2 | | | | | | | |
| pound 10 | | | | | | | | |
| Com- | 19.0 | | | | | | | |
| pound 11 | | | | | | | | |
| Com- | 43.4 | | | | | | | |
| pound 12 | | | | | | | | |
| Com- | 32.3 | 1628 | | | | | | |
| pound 13 | | | | | | | | |
| Com- | | | | | | | | |
| pound 14 | | | | | | | | |
| Com- | | | | | | | | |
| pound 15 | | | | | | | | |
| Com- | 7.40 | 400 | 10.7 | >1000 | 5.74 | 501 | 4.51 | 436 |
| pound 16 | | | | | | | | |
| Com- | | | | | | | | |
| pound 17 | | | | | | | | |
| Com- | 6.59 | 2143 | | | | | | |
| pound 18 | | | | | | | | |
| Com- | 55.0 | 2870 | | | | | | |
| pound 19 | | | | | | | | |
| Com- | 8.39 | | | | | | | |
| pound 22 | | | | | | | | |
| Com- | | | | | | | | |
| pound 25 | | | | | | | | |
| Com- | 10.1 | 369 | 13.5 | 411 | 8.85 | 154 | 8.56 | 172 |
| pound 26 | | | | | | | | |
| Com- | | | | | | | | |
| pound 33 | | | | | | | | |
| Com- | | | | | | | | |
| pound 34 | | | | | | | | |
| Com- | | | | | | | | |
| pound 35 | | | | | | | | |
| Com- | | | | | | | | |
| pound 36 | | | | | | | | |
| Com- | | | | | | | | |
| pound 37 | | | | | | | | |
| Com- | | | | | | | | |
| pound 38 | | | | | | | | |
| Com- | | | | | | | | |
| pound 41 | | | | | | | | |
| Com- | | | | | | | | |
| pound 42 | | | | | | | | |
| Com- | | | | | | | | |
| pound 43 | | | | | | | | |
| Com- | | | | | | | | |
| pound 44 | | | | | | | | |
| Com- | 5.12 | 329 | 10.4 | >1000 | 2.56 | 160 | 2.84 | 575 |
| pound 47 | | | | | | | | |
| Com- | 140 | | | | | | | |
| pound 48 | | | | | | | | |
| Com- | 8.60 | 315 | 25.2 | >1000 | 7.79 | 144 | 4.85 | 230 |
| pound 49 | | | | | | | | |
| Com- | | | | | | | | |
| pound 51 | | | | | | | | |
| Com- | | | | | | | | |
| pound 52 | | | | | | | | |
| Com- | 8.33 | | | | | | | |
| pound 53 | | | | | | | | |
| Com- | 29.40 | | | | | | | |
| pound 55 | | | | | | | | |
| Com- | | | | | | | | |
| pound 57 | | | | | | | | |
| Com- | | | | | | | | |
| pound 58 | | | | | | | | |
| Com- | 214 | | | | | | | |
| pound 59 | | | | | | | | |

### 3. Phospho-ERK1/2(THR202/TYR204) HTRF Assay

p-ERK (MAPK pathway) inhibition activity of each of compounds in a variety of K-Ras mutant and K-Ras WT cell lines indicated in Table 18 was evaluated. MKN-1 with K-Ras WT amplification is also a K-Ras dependent cell line.

TABLE 18

| | | Cell | | | |
|---|---|---|---|---|---|
| | | seeding | | | |
| | | density, | | | |
| | K-Ras | cells/ | Culture | Assay | Cell |
| Cell lines | mutation | well | medium | medium | incubator |
| AGS | G12D | 60000 | F-12K, | F-12K, | 37° C., |
| | | | 10% FBS | 0.1% FBS | 5% CO₂ |
| SW620 | G12V | 60000 | L-15, | L-15, | 37° C., |
| | | | 10% FBS | 0.1% FBS | 100% Air |
| NCI-H358 | G12C | 40000 | RPMI 1640, | RPMI 1640, | 37° C., |
| | | | 10% FBS | 0.1% FBS | 5% CO₂ |
| NCI-H747 | G13D | 40000 | RPMI 1640, | RPMI 1640, | 37° C., |
| | | | 10% FBS | 0.1% FBS | 5% CO₂ |
| RERF-LC-Ad1 | G12A | 30000 | RPMI 1640, | RPMI 1640, | 37° C., |
| | | | 10% FBS | 0.1% FBS | 5% CO₂ |
| TCC-PAN2 | G12R | 20000 | RPMI 1640, | RPMI 1640, | 37° C., |
| | | | 10% FBS | 0.1% FBS | 5% CO₂ |
| NCI-H460 | Q61H | 30000 | RPMI 1640, | PMI 1640, | 37° C., |
| | | | 10% FBS | 0.1% FBS | 5% CO₂ |
| MKN-1 | WT | 30000 | RPMI 1640, | RPMI 1640, | 37° C., |
| | | | 10% FBS | 0.1% FBS | 5% CO₂ |

Each of cells in culture medium was seeded in 96-well plates at density indicated in Table 18 and then put in a cell incubator to incubate overnight. The next day, the culture medium was removed and the compound diluted in assay medium was added in each well. After 2 hours incubation in a cell incubator, the assay medium in 96-well plates was removed, then 50 L of 1× blocking reagent-supplemented lysis buffer (Cisbio) was added and the plates were incubated at 25° C. for 45 min with shaking. 10 μL of cell lysates from the 96-well plates were transferred to a 384-well plate (Greiner) containing 2.5 L/well HTRF® pre-mixed antibodies (Cisbio 64AERPEH). The plate was incubated 4 hours at 25° C. and then read HTRF signals on Tecan Spark multimode microplate reader. The data were analyzed using a 4-parameter logistic model to calculate IC₅₀ values. The results are shown in the following Table 19:

TABLE 19

| | p-ERK, IC₅₀(nM) | | | | | | | |
|---|---|---|---|---|---|---|---|---|
| Compound | AGS | SW620 | NCI-H358 | NCI-H747 | RERF-LC-Ad1 | TCC-PAN2 | NCI-H460 | MKN-1 |
| Compound 1 | 23.8 | 9.18 | 8.93 | 6.50 | 8.32 | 760 | 63.6 | 10.6 |
| Compound 2 | 27.8 | 17.4 | | | | | | |
| Compound 3 | 4.74 | 2.69 | | | | | | |
| Compound 3A | 4.09 | 0.969 | 3.49 | 3.41 | 4.05 | 201 | 10.3 | 1.93 |
| Compound 4 | 27.0 | 10.0 | | 11.5 | 7.28 | 392 | 38.3 | 14.4 |
| Compound 5 | 13.8 | 7.37 | 17.4 | 26.2 | 20.0 | 780 | 113 | 27.4 |
| Compound 6 | 4.81 | 5.96 | 75.1 | 7.90 | 3.70 | 278 | 51.2 | 3.82 |
| Compound 7 | 7.23 | 6.36 | | | | | | |
| Compound 10 | | 64.7 | | | | | | |
| Compound 11 | | 23.0 | | | | | | |
| Compound 12 | | 64.4 | | | | | | |
| Compound 13 | | 34.8 | | | | | | |
| Compound 15 | 23.5 | 2.75 | | | | | | |
| Compound 16 | 28.1 | 2.28 | 60.1 | 20.1 | | ~1000 | 82.7 | 13.8 |
| Compound 17 | 182 | 54.8 | | | | | | |
| Compound 18 | | 49.3 | | | | | | |
| Compound 19 | | 89.2 | | | | | | |
| Compound 22 | | 28.6 | | | | | | |
| Compound 25 | 305 | 52.1 | | | | | | |
| Compound 26 | 36.2 | 8.74 | | 18.8 | | | | |
| Compound 33 | 38.6 | 8.39 | | | | | | |
| Compound 47 | 14.8 | 8.90 | 121 | 16.8 | | 455 | 45.6 | 18.6 |
| Compound 49 | 56.9 | 44.1 | 68.9 | 32.3 | | 925 | 71.0 | 25.9 |
| Compound 53 | | 165 | | | | | | |
| Compound 58 | | 65.3 | | | | | | |

### 4. Cell Growth Inhibition Assay

The cell growth inhibition activity of each of compounds was tested by performing cell growth inhibition assays on a variety of K-Ras mutant and K-Ras WT cell lines indicated in Table 20.

TABLE 20

| | | Cell | | | |
|---|---|---|---|---|---|
| | | seeding | | | |
| | K-Ras | density, | Culture | Assay | Cell |
| Cell lines | mutation | cells/well | medium | format | incubator |
| AGS | G12D | 500 | F-12K, | 2D | 37° C., |
| | | | 10% FBS | | 5% CO₂ |
| AsPC-1 | G12D | 1000 | RPMI 1640, | 2D | 37° C., |
| | | | 10% FBS | | 5% CO₂ |
| SW620 | G12V | 1500 | L-15, | 2D | 37° C., |
| | | | 10% FBS | | 100% Air |
| Capan-2 | G12V | 1500 | McCoy′5A, | 3D | 37° C., |
| | | | 10% FBS | | 5% CO₂ |
| NCI-H358 | G12C | 1500 | RPMI 1640, | 3D | 37° C., |
| | | | 10% FBS | | 5% CO₂ |
| NCI-H747 | G13D | 1500 | RPMI 1640, | 3D | 37° C., |
| | | | 10% FBS | | 5% CO₂ |
| RERF-LC-Ad1 | G12A | 3000 | RPMI 1640, | 3D | 37° C., |
| | | | 10% FBS | | 5% CO₂ |
| TCC-PAN2 | G12R | 500 | RPMI 1640, | 3D | 37° C., |
| | | | 10% FBS | | 5% CO₂ |
| NCI-H460 | Q61H | 300 | RPMI 1640, | 3D | 37° C., |
| | | | 10% FBS | | 5% CO₂ |
| MKN-1 | WT | 1500 | RPMI 1640, | 3D | 37° C., |
| | | | 10% FBS | | 5% CO₂ |

### 2D Cell Growth Inhibition Assays

Each of cells in culture medium was plated in TC-treated 96-well plates at a density indicated in Table 20 and incubated in a cell incubator overnight. The next day, each of compounds was diluted in culture medium and added to the plates. After 6 days incubation in cell incubator, the cell viability was detected by CellTiter-Glo® Cell Viability Assay kit (Promega). Luminescent signals were read on Tecan Spark multimode microplate reader and analyzed using a 4-parameter logistic model to calculate absolute IC₅₀ values. The results are shown in the following Table 21.

### 3D Cell Growth Inhibition Assays

Each of cells in culture medium was plated in ultra-low attachment-coated 96-well plates at a density indicated in Table 20 and incubated in a cell incubator overnight. The next day, each of compounds was diluted in culture medium and added to the plates. After 6 days incubation in cell incubator, the cell viability was detected by CellTiter-Glo® 3D Cell Viability Assay kit (Promega). Luminescent signals were read on Tecan Spark multimode microplate reader and analyzed using a 4-parameter logistic model to calculate absolute IC₅₀ values. The results are shown in the following Table 21.

TABLE 21

| | Cell Growth Inhibition, IC₅₀(nM) | | | | | | | | | |
|---|---|---|---|---|---|---|---|---|---|---|
| Compound | AGS | AsPC-1 | SW620 | Capan-2 | NCI-H358 | NCI-H747 | RERF-LC-Ad1 | TCC-PAN2 | NCI-H460 | MKN-1 |
| Compound 1 | 38.8 | 751 | 19.5 | 112 | 227 | 48.5 | 392 | >1000 | ~1000 | 61.0 |
| Compound 2 | 50.9 | 949 | 62.5 | | | | | | | |
| Compound 3 | 12.0 | 78.4 | 4.32 | | | | | | | |
| Compound 3A | 4.99 | 61.3 | 2.28 | 10.4 | 46.6 | 17.6 | 83.6 | 602 | 141 | 15.4 |
| Compound 3B | 87.8 | 1288 | 120 | | | | | | | |
| Compound 4 | 41.4 | 772 | 22.7 | 72.1 | | 59.6 | 802 | | 508 | 95.3 |
| Compound 5 | 34.7 | 467 | 14.1 | 124 | 165 | 89.1 | | >1000 | >1000 | 77.4 |
| Compound 6 | 17.7 | 53.5 | 21.2 | 121 | 14.5 | 22.9 | 284 | >1000 | 382 | 52.3 |
| Compound 7 | 4.69 | 41.9 | 2.98 | | | | | | | |
| Compound 10 | | | 123 | | | | | | | |
| Compound 11 | | | 50.5 | | | | | | | |
| Compound 12 | 406 | 2932 | 144 | | | | | | | |
| Compound 13 | | | 99.9 | | | | | | | |
| Compound 14 | | | 259 | | | | | | | |
| Compound 15 | 24.1 | 122 | 12.9 | | | | | | | |
| Compound 16 | 22.0 | 142 | 14.4 | 51.6 | 187 | 41.2 | | >1000 | 402 | 75.3 |
| Compound 17 | 94.2 | 555 | 110 | | | | | | | |
| Compound 18 | | | 135 | | | | | | | |
| Compound 22 | | | 41.6 | | | | | | | |
| Compound 25 | 243 | 2175 | 97.7 | | | | | | | |
| Compound 26 | 118 | 776 | 20.5 | | | 99.2 | | | | |
| Compound 33 | 97.9 | 703 | 71.0 | | | | | | | |
| Compound 47 | 38.1 | 476 | 42.8 | 231 | 239 | 57.4 | | >1000 | 727 | 70.0 |
| Compound 49 | 105 | 1056 | 15.1 | 76.4 | 211 | 79.7 | | >1000 | 646 | 137 |
| Compound 53 | | | 37.2 | | | | | | | |
| Compound 58 | | | 158 | | | | | | | |

### 5. Mouse Pharmacokinetic Study

The purpose of this study was to evaluate the pharmacokinetic properties of compounds in Balb/c mouse (♀) following single dose administration. Six mice were needed for each compound and the six mice were divided into two groups (n=3/group), group A and group B. Mice in group A were treated with a single 3 mg/kg dose of compound (iv). Mice in group B were treated with a single 10 mg/kg dose of compound (po). For each mouse in group A, blood samples were collected at the time point of 0.083, 0.5, 1, 2, 4 and 8 h post-dose. For each mouse in group B, blood samples were collected at the time point of 0.5, 1, 2, 4, 6 and 8 h post-dose. Blood samples were placed on ice until centrifugation to obtain plasma samples. The plasma samples were stored at −80° C. until analysis. The concentration of compound in plasma samples was determined using a LC-MS/MS method. The results are shown in the following Table 22.

For the prodrug, only the oral PK study was performed and the dosage was adjusted to be equal to 10 mg/kg of parent drug. The concentrations of both prodrug and parent drug in plasma were determined using a LC-MS/MS method. The results are shown in the following Table 23.

TABLE 22

| | 10 mg/kg, po | |
|---|---|---|
| | Cₘₐₓ | AUC₀₋₈ₕ |
| Compound | (ng/ml) | (ng · h/mL) |
| Compound 1 | 1118 | 3001 |
| Compound 2 | 73.6 | 151 |
| Compound 3 | 947 | 3148 |
| Compound 3A | 490 | 1258 |
| Compound 4 | 1953 | 5635 |
| Compound 5 | 91.3 | 168 |
| Compound 6 | 896 | 3454 |
| Compound 7 | 433 | 1376 |
| Compound 15 | 190 | 592 |
| Compound 16 | 165 | 696 |
| Compound 26 | 813 | 2588 |

TABLE 23

| | | Equal to 10 mg/kg of parent drug, po | |
|---|---|---|---|
| | | Cₘₐₓ | AUC₀₋₈ₕ |
| Compound | Analyte | (ng/mL) | (ng · h/mL) |
| Compound 8 | Compound 8 | 4963 | 18296 |
| | Compound 4 | 1091 | 4013 |
| Compound 20 | Compound 20 | 3483 | 11939 |
| | Compound 3A | 541 | 1463 |

What is claimed is:

1-58. (canceled)

59. A compound of Formula (IB), a stereoisomer thereof, a pharmaceutically acceptable salt thereof, a pharmaceutically acceptable salt of the stereoisomer thereof, a prodrug thereof, a deuterated molecule thereof or a conjugated form thereof: 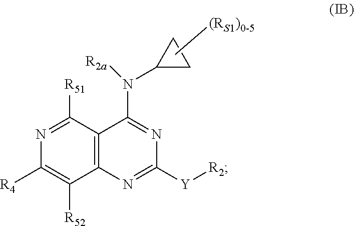
Wherein,
R₂ₐ is selected from hydrogen, deuterium, —C₁₋₁₀alkyl, haloC₁₋₁₀alkyl, haloC₁₋₁₀alkoxy, —C₂₋₁₀alkenyl, haloC₂₋₁₀alkenyl, —C₂₋₁₀alkynyl, haloC₂₋₁₀alkynyl, —N(R_{b})₂, —OR_{b}, —SR_{b}, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₁₀alkyl, haloC₁₋₁₀alkyl, haloC₁₋₁₀alkoxy, —C₂₋₁₀alkenyl, —C₂₋₁₀alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is optionally independently substituted with one or more substituents selected from deuterium, halogen, —C₁₋₆alkyl, haloC₁₋₆alkyl, haloC₁₋₆alkoxy, —C₂₋₆alkenyl, —C₂₋₆alkynyl, —CN, —NO₂, —N₃, oxo, —N(R_{c})₂, —OR_{c}, —SR_{c}, —S(═O)R_{d}, —S(═O)₂R_{d}, —C(═O)R_{d}, —C(═O)OR_{c}, —OC(═O)R_{d}, —C(═O)N(R_{c})₂, —NR_{c}C(═O)R_{d}, —OC(═O)OR_{c}, —NR_{c}C(═O)OR_{d}, —OC(═O)N(R_{c})₂, —NR_{c}C(═O)N(R_{c})₂, —S(═O)OR_{c}, —OS(═O)R_{d}, —S(═O)N(R_{c})₂, —NR_{c}S(═O)R_{d}, —S(═O)₂OR_{c}, —OS(═O)₂R_{d}, —S(═O)₂N(R_{c})₂, —NR_{c}S(═O)₂R_{d}, —OS(═O)₂OR_{c}, —NR_{c}S(═O)₂OR_{c}, —OS(═O)₂NR_{c}, —NR_{c}S(═O)₂N(R_{c})₂, —P(R_{c})₂, —P(═O)(R_{d})₂, 3-6 membered cycloalkyl, 3-6 membered cycloalkenyl, 3-6 membered cycloalkynyl, 3-10 membered heterocyclyl, 6-10 membered aryl or 5-10 membered heteroaryl;
R_{S1} at each occurrence is independently selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R₆₁)₂, —OR₆₁, —SR₆₁, —S(═O)R₆₂, —S(═O)₂R₆₂, —C(═O)R₆₂, —C(═O)OR₆₁, —OC(═O)R₆₂, —C(═O)N(R₆₁)₂, —NR₆₁C(═O)R₆₂, —OC(═O)OR₆₁, —NR₆₁C(═O)OR₆₁, —NR₆₁C(═S)OR₆₁, —OC(═O)N(R₆₁)₂, —NR₆₁C(═O)N(R₆₁)₂, —S(═O)OR₆₁, —OS(═O)R₆₂, —S(═O)N(R₆₁)₂, —NR₆₁S(═O)R₆₂, —S(═O)₂OR₆₁, —OS(═O)₂R₆₂, —S(═O)₂N(R₆₁)₂, —NR₆₁S(═O)₂R₆₂, —OS(═O)₂OR₆₁, —NR₆₁S(═O)₂OR₆₁, —OS(═O)₂N(R₆₁)₂, —NR₆₁S(═O)₂N(R₆₁)₂, —P(R₆₁)₂, —P(═O)(R₆₂)₂, 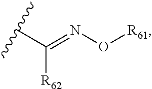
3-8 membered cycloalkyl, 3-8 membered cycloalkenyl, 3-8 membered cycloalkynyl, 4-8 membered heterocyclyl, 6-10 membered aryl, 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, 3-8 membered cycloalkyl, 3-8 membered cycloalkenyl, 3-8 membered cycloalkynyl, 3-8 membered heterocyclyl, 6-10 membered aryl or 5-10 membered heteroaryl is optionally independently substituted with one or more substituents selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R₆₃)₂, —OR₆₃, —SR₆₃, —S(═O)R₆₄, —S(═O)₂R₆₄, —C(═O)R₆₄, —C(═O)OR₆₄, —OC(═O)R₆₄, —C(═O)N(R₆₃)₂, —NR₆₃C(═O)R₆₄, —OC(═O)OR₆₃, —NR₆₃C(═O)OR₆₃, —NR₆₃C(═S)OR₆₃, —OC(═O)N(R₆₃)₂, —NR₆₃C(═O)N(R₆₃)₂, —S(═O)OR₆₃, —OS(═O)R₆₄, —S(═O)N(R₆₃)₂, —NR₆₃S(═O)R₆₄, —S(═O)₂OR₆₃, —OS(═O)₂R₆₄, —S(═O)₂N(R₆₃)₂, —NR₆₃S(═O)₂R₆₄, —OS(═O)₂OR₆₃, —NR₆₃S(═O)₂OR₆₃, —OS(═O)₂N(R₆₃)₂, —NR₆₃S(═O)₂N(R₆₃)₂, —P(R₆₃)₂, —P(═O)(R₆₄)₂, 3-6 membered cycloalkyl, 3-6 membered cycloalkenyl, 3-6 membered cycloalkynyl, 3-6 membered heterocyclyl, 6-10 membered aryl or 5-10 membered heteroaryl;
Optionally, two R_{S1} together with the carbon atom to which they are both attached form 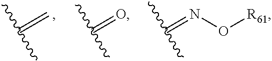
a 3-10 membered carbocyclic ring or a 3-10 heterocyclic ring; wherein, said 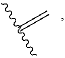
3-10 membered carbocylic ring or 3-10 heterocyclic ring is optionally substituted with one or more R_{16c};
Optionally, two adjacent R_{S1} together with the carbon atoms to which they are respectively attached form a 3-10 membered carbocyclic ring, a 3-10 membered heterocyclic ring, a 6-10 membered aryl ring or a 5-10 membered heteroaryl ring, wherein, each of rings is independently optionally substituted with one or more R₁₆ₐ;
Optionally, two nonadjacent R_{S1} are connected together to form a bridge containing 0, 1, 2, 3, 4, 5 or 6 carbon atoms, wherein, each of the carbon atoms in the bridge is optionally replaced by 1 or 2 heteroatoms selected from N, O, S, S═O or S(═O)₂; the hydrogen on the each of carbon atoms or N atoms is optionally independently substituted with Rᵢ₆ₑ;
Y is a bond, O, S, S(═O), S(═O)₂ or NR₆ₐ;
R₂ is selected from -L₅-(3-12 membered heterocyclyl), -L₅-(3-12 membered cycloalkyl), -L₅-(6-12 member aryl), -L₅-(5-12 membered heteroaryl), -L₅—N(R₇ₐ)₂, or 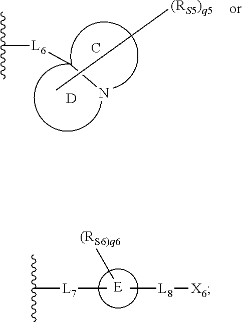
Each of L₅ at each occurrence is independently selected from a bond or C₁₋₁₀ alkylene optionally substituted with one or more R₁₆ₙ;
Said 3-12 membered heterocyclyl in -L₅-(3-12 membered heterocyclyl) is optionally substituted with one or more R₁₆ₒ;
Said 3-12 membered cycloalkyl in -L₅-(3-12 membered cycloalkyl) is optionally substituted with one or more R₁₆ₒ;
Said 6-12 member aryl in -L₅-(6-12 member aryl) is optionally substituted with one or more R₁₆ₒ;
Said 5-12 membered heteroaryl in -L₅-(5-12 membered heteroaryl) is optionally substituted with one or more R₁₆ₒ;
L₆ is selected from a bond or C₁₋₁₀ alkylene optionally substituted with one or more R₁₆ₚ;
L₇ is selected from a bond or C₁₋₁₀ alkylene optionally substituted with one or more R_{16q};
L₈ is selected from a bond or C₁₋₁₀ alkylene optionally substituted with one or more R₁₆ᵣ;
Ring C or ring D is a 3-10 membered heterocyclic ring which is optionally further contains 1, 2, or 3 heteroatoms selected from N, O or S;
Ring E is selected from a 3-10 membered carbocyclic ring or a 3-10 membered heterocyclic ring; wherein the moiety of -L₇- and -L₈-X₆ are attached to the same atom or different atoms of the ring E;
X₆ is selected from —N(R₆₅)₂, —OR₆₅, —SR₆₅, 3-10 membered heterocyclyl, or 5-10 membered heteroaryl, wherein said 3-10 membered heterocyclyl or 5-10 membered heteroaryl is optionally independently substituted with one or more R₁₆ₛ;
Each of R_{S5} at each occurrence is independently selected from deuterium, halogen, —C₁₋₆alkyl, haloC₁₋₆alkyl, haloC₁₋₆alkoxy, —C₂₋₆-alkenyl, haloC₂-alkenyl, —C₂-alkynyl, haloC₂-alkynyl, —CN, —NO₂, —N₃, oxo, —N(R₆₆)₂, —OR₆₆, —SR₆₆, —S(═O)R₆₇, —S(═O)₂R₆₇, —C(═O)R₆₇, —C(═O)OR₆₆, —OC(═O)R₆₇, —C(═O)N(R₆₆)₂, —NR₆₆C(═O)R₆₇, —OC(═O)OR₆₆, —NR₆₆C(═O)OR₆₆, —NR₆₆C(═S)OR₆₆, —OC(═O)N(R₆₆)₂, —NR₆₆C(═O)N(R₆₆)₂, —S(═O)OR₆₆, —OS(═O)R₆₇, —S(═O)N(R₆₆)₂, —NR₆₆S(═O)R₆₇, —S(═O)₂OR₆₆, —OS(═O)₂R₆₇, —S(═O)₂N(R₆₆)₂, —NR₆₆S(═O)₂R₆₇, —OS(═O)₂OR₆₆, —NR₆₆S(═O)₂OR₆₆, —OS(═O)₂N(R₆₆)₂, —NR₆₆S(═O)₂N(R₆₆)₂, —P(R₆₆)₂, —P(═O)(R₆₇)₂, 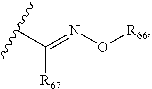
3-8 membered cycloalkyl, 3-8 membered cycloalkenyl, 3-8 membered cycloalkynyl, 4-8 membered heterocyclyl, 6-10 membered aryl, 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂-alkynyl, 3-8 membered cycloalkyl, 3-8 membered cycloalkenyl, 3-8 membered cycloalkynyl, 3-8 membered heterocyclyl, 6-10 membered aryl or 5-10 membered heteroaryl is optionally independently substituted with one or more substituents selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂-alkynyl, —CN, —NO₂, —N₃, oxo, —N(R₆₈)₂, —OR₆₈, —SR₆₈, —S(═O)R₆₉, —S(═O)₂R₆₉, —C(═O)R₆₉, —C(═O)OR₆₈, —OC(═O)R₆₉, —C(═O)N(R₆₈)₂, —NR₆₈C(═O)R₆₉, —OC(═O)OR₆₈, —NR₆₈C(═O)OR₆₈, —NR₆₈C(═S)OR₆₈, —OC(═O)N(R₆₈)₂, —NR₆₈C(═O)N(R₆₈)₂, —S(═O)OR₆₈, —OS(═O)R₆₉, —S(═O)N(R₆₈)₂, —NR₆₈S(═O)R₆₉, —S(═O)₂OR₆₈, —OS(═O)₂R₆₉, —S(═O)₂N(R₆₈)₂, —NR₆₈S(═O)₂R₆₉, —OS(═O)₂OR₆₈, —NR₆₈S(═O)₂OR₆₈, —OS(═O)₂N(R₆₈)₂, —NR₆₈S(═O)₂N(R₆₈)₂, —P(R₆₈)₂, —P(═O)(R₆₉)₂, 3-6 membered cycloalkyl, 3-6 membered cycloalkenyl, 3-6 membered cycloalkynyl, 3-6 membered heterocyclyl, 6-10 membered aryl or 5-10 membered heteroaryl;
Optionally, two R_{S5} together with the carbon atom to which they are both attached form a 3-10 membered carbocyclic ring or a 3-10 heterocyclic ring; wherein, said 3-10 membered carbocylic ring or 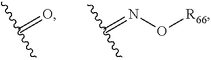
3-10 heterocyclic ring is optionally substituted with one or more R₁₆ₜ;
Optionally, two adjacent R_{S5} together with the carbon atoms to which they are respectively attached form a 3-10 membered carbocyclic ring, a 3-10 membered heterocyclic ring, a 6-10 membered aryl ring or a 5-10 membered heteroaryl ring, wherein, each of rings is independently optionally substituted with one or more R₁₆ᵤ;
Optionally, two nonadjacent R_{S5} are connected together to form a bridge containing 0, 1, 2, 3, 4, 5 or 6 carbon atoms, wherein, each of the carbon atoms in the bridge is optionally replaced by 1 or 2 heteroatoms selected from N, O, S, S═O or S(═O)₂; the hydrogen on the each of carbon atoms or N atoms is optionally independently substituted with R₁₆ᵥ;
q₅ is selected from 0, 1, 2, 3, 4, 5 or 6;
Each of R_{S6} at each occurrence is independently selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R₇₁)₂, —OR₇₁, —SR₇₁, —S(═O)R₇₂, —S(═O)₂R₇₁, —C(═O)R₇₂, —C(═O)OR₇₁, —OC(═O)R₇₂, —C(═O)N(R₇₁)₂, —NR₇₁C(═O)R₇₂, —OC(═O)OR₇₁, —NR₇₁C(═O)OR₇₁, —OC(═O)N(R₇₁)₂, —NR₇₁C(═O)N(R₇₁)₂, —S(═O)OR₇₁, —OS(═O)R₇₂, —S(═O)N(R₇₁)₂, —NR₇₁S(═O)R₇₂, —S(═O)₂OR₇₁, —OS(═O)₂R₇₂, —S(═O)₂N(R₇₁)₂, —NR₇₁S(═O)₂R₇₂, —OS(═O)₂OR₇₁, —NR₇₁S(═O)₂OR₇₂, —OS(═O)₂N(R₇₁)₂, —NR₇₁S(═O)₂N(R₇₁)₂, —P(R₇₁)₂, —P(═O)(R₇₂)₂, 3-6 membered cycloalkyl, 3-6 membered cycloalkenyl, 3-6 membered cycloalkynyl, 3-6 membered heterocyclyl, 6-10 membered aryl or 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, 3-6 membered cycloalkyl, 3-6 membered cycloalkenyl, 3-6 membered cycloalkynyl, 3-6 membered heterocyclyl, 6-10 membered aryl or 5-10 membered heteroaryl is optionally independently substituted with one or more substituents selected from deuterium, halogen, —C₁₋₆alkyl, haloC₁₋₆ alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R₇₃)₂, —OR₇₃, —SR₇₃, —S(═O)R₇₄, —S(═O)₂R₇₃, —C(═O)R₇₄, —C(═O)OR₇₃, —OC(═O)R₇₄, —C(═O)N(R₇₃)₂, —NR₇₃C(═O)R₇₄, —OC(═O)OR₇₃, —NR₇₃C(═O)OR₇₃, —OC(═O)N(R₇₃)₂, —NR₇₃C(═O)N(R₇₃)₂, —S(═O)OR₇₃, —OS(═O)R₇₄, —S(═O)N(R₇₃)₂, —NR₇₃S(═O)R₇₄, —S(═O)₂OR₇₃, —OS(═O)₂R₇₄, —S(═O)₂N(R₇₃)₂, —NR₇₃S(═O)₂R₇₄, —OS(═O)₂OR₇₃, —NR₇₃S(═O)₂OR₇₄, —OS(═O)₂N(R₇₃)₂, —NR₇₃S(═O)₂N(R₇₃)₂, —P(R₇₃)₂, —P(═O)(R₇₄)₂, 3-6 membered cycloalkyl, 3-6 membered cycloalkenyl, 3-6 membered cycloalkynyl, 3-6 membered heterocyclyl, 6-10 membered aryl or 5-10 membered heteroaryl;
q₆ is selected from 0, 1, 2, 3, 4, 5 or 6;
R₄ is selected from 6-10 membered aryl, 5-10 membered heteroaryl, 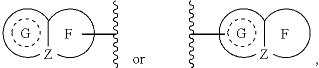
wherein said 6-10 membered aryl, 5-10 membered heteroaryl, 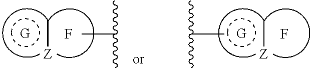
is optionally independently substituted with one or more R₄₁;
Z at each occurrence is independently selected from C or N;
Ring G at each occurrence is independently selected from a 6 membered aryl ring or a 5-6 membered heteroaryl ring and ring F at each occurrence is a 3-10 membered carbocyclic ring or a 3-10 membered heterocyclic ring when Z is selected from C;
Ring G at each occurrence is selected from a 5-6 membered heteroaryl ring and ring F at each occurrence is a 3-10 membered heterocyclic ring when Z is selected from N;
R₄₁ at each occurrence is independently selected from deuterium, halogen, —C₁₋₁₀alkyl, haloC₁₋₁₀alkyl, haloC₁₋₁₀alkoxy, —C₂₋₁₀alkenyl, haloC₂₋₁₀alkenyl, —C₂₋₁₀alkynyl, haloC₂₋₁₀alkynyl, —CN, —NO₂, —N₃, oxo, —N(R₇₅)₂, —OR₇₅, —SR₇₅, —S(═O)R₇₆, —S(═O)₂R₇₆, —C(═O)R₇₆, —C(═O)OR₇₅, —OC(═O)R₇₆, —C(═O)N(R₇₅)₂, —NR₇₅C(═O)R₇₆, —OC(═O)OR₇₅, —NR₇₅C(═O)OR₇₅, —OC(═O)N(R₇₅)₂, —NR₇₅C(═O)N(R₇₅)₂, —S(═O)OR₇₅, —OS(═O)R₇₆, —S(═O)N(R₇₅)₂, —NR₇₅S(═O)R₇₆, —S(═O)₂OR₇₅, —OS(═O)₂R₇₆, —S(═O)₂N(R₇₅)₂, —NR₇₅S(═O)₂R₇₆, —OS(═O)₂OR₇₅, —NR₇₅S(═O)₂OR₇₅, —OS(═O)₂N(R₇₅)₂, —NR₇₅S(═O)₂N(R₇₅)₂, —P(R₇₅)₂, —P(═O)(R₇₅)₂, 3-10 membered cycloalkyl, 3-10 membered cycloalkenyl, 3-10 membered cycloalkynyl, 3-10 membered heterocyclyl, 6-10 membered aryl or 5-10 membered heteroaryl, wherein said —C₁₋₁₀alkyl, haloC₁₋₁₀alkyl, haloC₁₋₁₀alkoxy, —C₂₋₁₀alkenyl, haloC₂₋₁₀alkenyl, —C₂₋₁₀alkynyl, haloC₂₋₁₀alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is optionally independently substituted with one or more substituents selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R₇₇)₂, —OR₇₇, —SR₇₇, —S(═O)R₇₈, —S(═O)₂R₇₈, —C(═O)R₇₈, —C(═O)OR₇₇, —OC(═O)R₇₈, —C(═O)N(R₇₇)₂, —NR₇₇C(═O)R₇₈, —OC(═O)OR₇₇, —NR₇₇C(═O)OR₇₇, —OC(═O)N(R₇₇)₂, —NR₇₇C(═O)N(R₇₇)₂, —S(═O)OR₇₇, —OS(═O)R₇₈, —S(═O)N(R₇₇)₂, —NR₇₇S(═O)R₇₈, —S(═O)₂OR₇₇, —OS(═O)₂R₇₈, —S(═O)₂N(R₇₇)₂, —NR₇₇S(═O)₂R₇₈, —OS(═O)₂OR₇₇, —NR₇₇S(═O)₂OR₇₇, —OS(═O)₂N(R₇₇)₂, —NR₇₇S(═O)₂N(R₇₇)₂, —P(R₇₇)₂, —P(═O)(R₇₈)₂, 3-6 membered cycloalkyl, 3-6 membered cycloalkenyl, 3-6 membered cycloalkynyl, 3-6 membered heterocyclyl, 6-10 membered aryl or 5-10 membered heteroaryl;
Each of (R₅₁ and R₅₂) is independently selected from hydrogen, deuterium, halogen, —C₁₋₁₀alkyl, haloC₁₋₁₀alkyl, haloC₁₋₁₀alkoxy, —C₂₋₁₀alkenyl, haloC₂₋₁₀alkenyl, —C₂₋₁₀alkynyl, haloC₂₋₁₀alkynyl, —CN, —NO₂, —N₃, oxo, —N(R₈₁)₂, —OR₈₁, —SR₈₁, —S(═O)R₈₂, —S(═O)₂R₈₂, —C(═O)R₈₂, —C(═O)OR₈₁, —OC(═O)R₈₂, —C(═O)N(R₈₁)₂, —NR₈₁C(═O)R₈₂, —OC(═O)OR₈₁, —NR₈₁C(═O)OR₈₁, —OC(═O)N(R₈₁)₂, —NR₈₁C(═O)N(R₈₁)₂, —S(═O)OR₈₁, —OS(═O)R₈₂, —S(═O)N(R₈₁)₂, —NR₈₁S(═O)R₈₂, —S(═O)₂OR₈₁, —OS(═O)₂R₈₂, —S(═O)₂N(R₈₁)₂, —NR₈₁S(═O)₂R₈₂, —OS(═O)₂OR₈₁, —NR₈₁S(═O)₂OR₈₁, —OS(═O)₂N(R₈₁)₂, —NR₈₁S(═O)₂N(R₈₁)₂, —P(R₈₁)₂, —P(═O)(R₈₂)₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₁₀alkyl, haloC₁₋₁₀alkyl, haloC₁₋₁₀alkoxy, —C₂₋₁₀alkenyl, haloC₂₋₁₀alkenyl, —C₂₋₁₀alkynyl, haloC₂₋₁₀alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is optionally independently substituted with one or more substituents selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R₈₃)₂, —OR₈₃, —SR₈₃, —S(═O)R₈₄, —S(═O)₂R₈₄, —C(═O)R₈₄, —C(═O)OR₈₃, —OC(═O)R₈₃, —C(═O)N(R₈₃)₂, —NR₈₃C(═O)R₈₄, —OC(═O)OR₈₃, —NR₈₃C(═O)OR₈₃, —OC(═O)N(R₈₃)₂, —NR₈₃C(═O)N(R₈₄)₂, —S(═O)OR₈₃, —OS(═O)R₈₄, —S(═O)N(R₈₃)₂, —NR₈₃S(═O)R₈₄, —S(═O)₂OR₈₃, —OS(═O)₂R₈₄, —S(═O)₂N(R₈₃)₂, —NR₈₃S(═O)₂R₈₄, —OS(═O)₂OR₈₃, —NR₈₃S(═O)₂OR₈₃, —OS(═O)₂N(R₈₃)₂, —NR₈₃S(═O)₂N(R₈₃)₂, —P(R₈₃)₂, —P(═O)(R₈₄)₂, 3-6 membered cycloalkyl, 3-6 membered cycloalkenyl, 3-6 membered cycloalkynyl, 3-6 membered heterocyclyl, 6-10 membered aryl or 5-10 membered heteroaryl;
Each of (R₆ₐ, R₇ₐ, R₆₁, R₆₃, R₆₅, R₆₆, R₆₈, R₇₁, R₇₃, R₇₅, R₇₇, R₈₁ and R₈₃) at each occurrence is independently selected from hydrogen, deuterium, halogen, —C₁₋₁₀alkyl, haloC₁₋₁₀alkyl, —C₂₋₁₀alkenyl, —C₂₋₁₀alkynyl, —S(═O)Rₐ, —S(═O)₂Rₐ, —C(═O)Rₐ, —C(═O)ORₐ, —C(═O)N(Rₐ)₂, —S(═O)ORₐ, —S(═O)N(Rₐ)₂, —S(═O)₂ORₐ, —S(═O)₂N(Rₐ)₂, —P(═O)(Rₐ)₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₁₀alkyl, haloC₁₋₁₀alkyl, —C₂₋₁₀alkenyl, —C₂₋₁₀alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is optionally independently substituted with one or more substituents selected from deuterium, halogen, —C₁₋₆alkyl, haloC₁₋₆ alkyl, haloC₁₋₆alkoxy, —C₂₋₆alkenyl, —C₂₋₆alkynyl, —CN, —NO₂, —N₃, oxo, —N(R_{c})₂, —OR_{c}, —SR, —S(═O)R_{d}, —S(═O)₂R_{d}, —C(═O)R_{d}, —C(═O)OR_{c}, —OC(═O)R_{d}, —C(═O)N(R_{c})₂, —NR_{c}C(═O)R_{d}, —OC(═O)OR_{c}, —NR_{c}C(═O)OR_{d}, —OC(═O)N(R_{c})₂, —NR_{c}C(═O)N(R_{c})₂, —S(═O)OR_{c}, —OS(═O)R_{d}, —S(═O)N(R_{c})₂, —NR_{c}S(═O)R_{d}, —S(═O)₂OR_{c}, —OS(═O)₂R_{d}, —S(═O)₂N(R_{c})₂, —NR_{c}S(═O)₂R_{d}, —OS(═O)₂OR_{c}, —NR_{c}S(═O)₂OR_{c}, —OS(═O)₂NR_{c}, —NR_{c}S(═O)₂N(R_{c})₂, —P(R_{c})₂, —P(═O)(R_{d})₂, 3-6 membered cycloalkyl, 3-6 membered cycloalkenyl, 3-6 membered cycloalkynyl, 3-6 membered heterocyclyl, 6-10 membered aryl or 5-10 membered heteroaryl;
Optionally, each of (two R₇ₐ, two R₆₁, two R₆₃, two R₆₅, two R₆₆, two R₆₈, two R₇₁, two R₇₃, two R₂₅, two R₇₇, two R₈₁, and two R₈₃) independently together with the nitrogen atom to which they are both attached forms a 3-20 membered heterocyclic ring or a 5-10 membered heteroaryl ring, wherein, said 3-20 membered heterocyclic ring or 5-10 membered heteroaryl ring is optionally independently substituted with one or more R_{16w};
Each of (R₆₂, R₆₄, R₆₂, R₆₉, R₇₂, R₇₄, R₇₆, R₂₈, R₈₂ and R₈₄) at each occurrence is independently selected from hydrogen, deuterium, —C₁₋₁₀alkyl, haloC₁₋₁₀alkyl, haloC₁₋₁₀alkoxy, —C₂₋₁₀alkenyl, haloC₂₋₁₀alkenyl, —C₂₋₁₀alkynyl, haloC₂₋₁₀alkynyl, —N(R_{b})₂, —OR_{b}, —SR_{b}, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₁₀alkyl, haloC₁₋₁₀alkyl, haloC₁₋₁₀alkoxy, —C₂₋₁₀alkenyl, —C₂₋₁₀alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is optionally independently substituted with one or more substituents selected from deuterium, halogen, —C₁₋₆alkyl, haloC₁₋₆alkyl, haloC₁₋₆alkoxy, —C₂₋₆alkenyl, —C₂₋₆alkynyl, —CN, —NO₂, —N₃, oxo, —N(R_{c})₂, —OR_{c}, —SR, —S(═O)R_{d}, —S(═O)₂R_{d}, —C(═O)R_{d}, —C(═O)OR, —OC(═O)R_{d}, —C(═O)N(R_{c})₂, —NR_{c}C(═O)R_{d}, —OC(═O)OR_{c}, —NR_{c}C(═O)OR_{d}, —OC(═O)N(R_{c})₂, —NR_{c}C(═O)N(R_{c})₂, —S(═O)OR_{c}, —OS(═O)R_{d}, —S(═O)N(R_{c})₂, —NR_{c}S(═O)R_{d}, —S(═O)₂OR_{c}, —OS(═O)₂R_{d}, —S(═O)₂N(R_{c})₂, —NR_{c}S(═O)₂R_{d}, —OS(═O)₂OR_{c}, —NR_{c}S(═O)₂OR_{c}, —OS(═O)₂NR_{c}, —NR_{c}S(═O)₂N(R_{c})₂, —P(R_{c})₂, —P(═O)(R_{d})₂, 3-6 membered cycloalkyl, 3-6 membered cycloalkenyl, 3-6 membered cycloalkynyl, 3-10 membered heterocyclyl, 6-10 membered aryl or 5-10 membered heteroaryl;
Each of (Rₐ, R_{b}, R_{c} and R_{d}) at each occurrence is independently selected from hydrogen, deuterium, —C₁₋₆alkyl, haloC₁₋₆alkyl, haloC₁₋₆alkoxy, —C₂₋₆alkenyl, —C₂₋₆alkynyl, 3-6 membered cycloalkyl, 3-6 membered cycloalkenyl, 3-6 membered cycloalkynyl, 3-6 membered heterocyclyl, 6-10 membered aryl or 5-10 membered heteroaryl; wherein said —C₁₋₆alkyl, haloC₁₋₆alkyl, haloC₁₋₆alkoxy, —C₂₋₆alkenyl, —C₂₋₆alkynyl, 3-6 membered cycloalkyl, 3-6 membered cycloalkenyl, 3-6 membered cycloalkynyl, 3-6 membered heterocyclyl, 6-10 membered aryl or 5-10 membered heteroaryl is optionally independently substituted with one or more R₁₆ₓ;
Optionally, each of (two Rₐ, two R_{b} and two R_{c}) independently together with the atom to which they are both attached forms a 3-6 membered heterocyclic ring, wherein said 3-6 membered heterocyclic ring is independently optionally substituted with one or more R_{16y};
Each of (R_{16c}, R_{16d}, R₁₆ₑ, R₁₆ₙ, R₁₆ₒ, R₁₆ₚ, R_{16q}, R₁₆ᵣ, R₁₆ₛ, R₁₆ₜ, R₁₆ᵤ, R₁₆ᵥ, R_{16w}, R₁₆ₓ and R_{16y}) at each occurrence is independently selected from deuterium, halogen, —C₁₋₆alkyl, haloC₁₋₆alkyl, haloC₁₋₆alkoxy, —C₂₋₆alkenyl, —C₂₋₆alkynyl, —CN, —NO₂, —N₃, oxo, —NH₂, —NH(C₁₋₆alkyl), —N(C₁₋₆alkyl)₂, —OH, —O(C₁₋₆alkyl), —SH, —S(C₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆ alkyl)C(═O)(C₁₋₆alkyl), —OC(═O)O(C₁₋₆alkyl), —NHC(═O)(OC₁₋₆alkyl), —N(C₁₋₆alkyl)C(═O)(OC₁₋₆alkyl), —OC(═O)NH(C₁₋₆alkyl), —OC(═O)N(C₁₋₆alkyl)₂, —NHC(═O)NH₂, —NHC(═O)NH(C₁₋₆alkyl), —NHC(═O)N(C₁₋₆ alkyl)₂, —N(C₁₋₆alkyl)C(═O)NH₂, —N(C₁₋₆alkyl)C(═O)NH(C₁₋₆alkyl), —N(C₁₋₆alkyl)C(═O)N(C₁₋₆alkyl)₂, —S(═O)(OC₁₋₆alkyl), —OS(═O)(C₁₋₆alkyl), —S(═O)NH₂, —S(═O)NH(C₁₋₆alkyl), —S(═O)N(C₁₋₆alkyl)₂, —NHS(═O)(C₁₋₆ alkyl), —N(C₁₋₆alkyl)S(═O)(C₁₋₆alkyl), —S(═O)₂(OC₁₋₆alkyl), —OS(═O)₂(C₁₋₆alkyl), —S(═O)₂NH₂, —S(═O)₂NH(C₁₋₆ alkyl), —S(═O)₂N(C₁₋₆alkyl)₂, —NHS(═O)₂(C₁₋₆alkyl), —N(C₁₋₆alkyl)S(═O)₂(C₁₋₆alkyl), —OS(═O)₂O(C₁₋₆alkyl), —NHS(═O)₂O(C₁₋₆alkyl), —N(C₁₋₆alkyl)S(═O)₂O(C₁₋₆alkyl), —OS(═O)₂NH₂, —OS(═O)₂NH(C₁₋₆alkyl), —OS(═O)₂N(C₁₋₆alkyl)₂, —NHS(═O)₂NH₂, —NHS(═O)₂NH(C₁₋₆alkyl), —NHS(═O)₂N(C₁₋₆alkyl)₂, —N(C₁₋₆ alkyl)S(═O)₂NH₂, —N(C₁₋₆alkyl)S(═O)₂NH(C₁₋₆alkyl), —N(C₁₋₆alkyl)S(═O)₂N(C₁₋₆alkyl)₂, —PH(C₁₋₆alkyl), —P(C₁₋₆ alkyl)₂, —P(═O)H(C₁₋₆alkyl), —P(═O)(C₁₋₆alkyl)₂, 3-6 membered cycloalkyl, 3-6 membered cycloalkenyl, 3-6 membered cycloalkynyl, 3-6 membered heterocyclyl, 6-10 membered aryl or 5-10 membered heteroaryl;
wherein, said —C₁₋₆alkyl, haloC₁₋₆alkyl, haloC₁₋₆alkoxy, —C₂₋₆alkenyl, —C₂₋₆alkynyl, 3-6 membered cycloalkyl, 3-6 membered cycloalkenyl, 3-6 membered cycloalkynyl, 3-6 membered heterocyclyl, 6-10 membered aryl or 5-10 membered heteroaryl is optionally substituted with one or more substituents selected from deuterium, halogen, —C₁₋₃alkyl, haloC₁₋₃alkyl, haloC₁₋₃alkoxy, —C₂₋₃alkenyl, —C₂₋₃alkynyl, —CN, —NO₂, —N₃, oxo, —NB₂, —NH(C₁₋₃alkyl), —N(C₁₋₃alkyl)₂, —OH, —O(C₁₋₃alkyl), —SH, —S(C₁₋₃alkyl), —S(═O)(C₁₋₃alkyl), —S(═O)₂(C₁₋₃alkyl), —C(═O)(C₁₋₃alkyl), —C(═O)OH, —C(═O)(OC₁₋₃alkyl), —OC(═O)(C₁₋₃alkyl), —C(═O)NH₂, —C(═O)NH(C₁₋₃alkyl), —C(═O)N(C₁₋₃alkyl)₂, —NHC(═O)(C₁₋₃alkyl), —N(C₁₋₃alkyl)C(═O)(C₁₋₃alkyl), —OC(═O)O(C₁₋₃alkyl), —NHC(═O)(OC₁₋₃alkyl), —N(C₁₋₃alkyl)C(═O)(OC₁₋₃alkyl), —OC(═O)NH(C₁₋₃alkyl), —OC(═O)N(C₁₋₃alkyl)₂, —NHC(═O)NH₂, —NHC(═O)NH(C₁₋₃alkyl), —NHC(═O)N(C₁₋₃alkyl)₂, —N(C₁₋₃alkyl)C(═O)NH₂, —N(C₁₋₃alkyl)C(═O)NH(C₁₋₃alkyl), —N(C₁₋₃ alkyl)C(═O)N(C₁₋₃alkyl)₂, —S(═O)(OC₁₋₃alkyl), —OS(═O)(C₁₋₃alkyl), —S(═O)NH₂, —S(═O)NH(C₁₋₃alkyl), —S(═O)N(C₁₋₃alkyl)₂, —NHS(═O)(C₁₋₃alkyl), —N(C₁₋₃alkyl)S(═O)(C₁₋₃alkyl), —S(═O)₂(OC₁₋₃alkyl), —OS(═O)₂(C₁₋₃alkyl), —S(═O)₂NH₂, —S(═O)₂NH(C₁₋₃alkyl), —S(═O)₂N(C₁₋₃alkyl)₂, —NHS(═O)₂(C₁₋₃alkyl), —N(C₁₋₃ alkyl)S(═O)₂(C₁₋₃alkyl), —OS(═O)₂O(C₁₋₃alkyl), —NHS(═O)₂O(C₁₋₃alkyl), —N(C₁₋₃alkyl)S(═O)₂O(C₁₋₃alkyl), —OS(═O)₂NH₂, —OS(═O)₂NH(C₁₋₃alkyl), —OS(═O)₂N(C₁₋₃alkyl)₂, —NHS(═O)₂NH₂, —NHS(═O)₂NH(C₁₋₃alkyl), —NHS(═O)₂N(C₁₋₃alkyl)₂, —N(C₁₋₃alkyl)S(═O)₂NH₂, —N(C₁₋₃alkyl)S(═O)₂NH(C₁₋₃alkyl), —N(C₁₋₃alkyl)S(═O)₂N(C₁₋₃alkyl)₂, —PH(C₁₋₃alkyl), —P(C₁₋₃alkyl)₂, —P(═O)H(C₁₋₃alkyl), —P(═O)(C₁₋₃alkyl)₂, 3-6 membered cycloalkyl, 3-6 membered cycloalkenyl, 3-6 membered cycloalkynyl, 3-6 membered heterocyclyl, 6 membered aryl or 5-6 membered heteroaryl;
Each of (heterocyclyl and heteroaryl) at each occurrence is independently contain 1, 2, 3 or 4 heteroatoms selected from N, O, S, S(═O) or S(═O)₂.

60. The compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of claim 59, wherein, the compound is selected from any one of the following formulas: 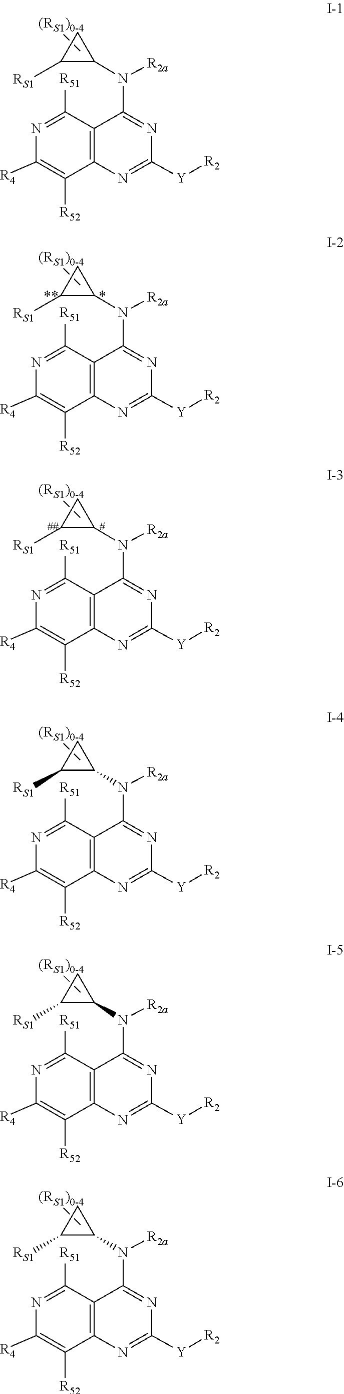 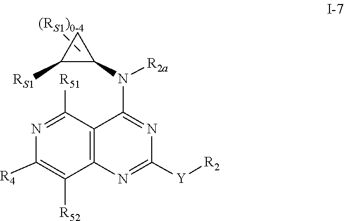
Wherein:
R_{S1} linked with the carbon atom indicated with ** and NR₂ₐ linked with the carbon atom indicated with * are in a trans configuration;
R_{S1} linked with the carbon atom indicated with ## and NR₂ₐ linked with the carbon atom indicated with # are in a cis configuration.

61. The compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of claim 59, wherein, R₂ₐ is selected from hydrogen, deuterium, —C₁₋₆alkyl, haloC₁₋₆alkyl, —C₁₋₆alkoxy, —C(═O)C₁₋₆alkyl, 3-6 membered cycloalkyl, 3-6 membered heterocyclyl containing 1 or 2 heteroatoms selected from N, O, S, S(═O) or S(═O)₂, phenyl, or 5-6 membered heteroaryl containing 1 or 2 heteroatoms selected from N, O or S; wherein said —C₁₋₆alkyl, haloC₁₋₆alkyl, —C₁₋₆alkoxy, —C(═O)C₁₋₆alkyl, 3-6 membered cycloalkyl, 3-6 membered heterocyclyl, phenyl or 5-6 membered heteroaryl is optionally independently substituted with one or more substituents selected from deuterium, halogen, —C₁₋₃alkyl, haloC₁₋₃alkyl, —C₂₋₃alkenyl, —C₂₋₃alkynyl, —CN, —NH₂, —NH(C₁₋₃alkyl), —N(C₁₋₃alkyl)₂, —OH, —OC₁₋₃alkyl, —SH, —SC₁₋₃alkyl, —C(═O)C₁₋₃alkyl, —C(═O)OH, —C(═O)OC₁₋₃alkyl, —OC(═O)C₁₋₃alkyl, 3-6 membered cycloalkyl or 3-6 membered heterocyclyl containing 1 or 2 heteroatoms selected from N, O, S, S(═O) or S(═O)₂, phenyl, or 5-6 membered heteroaryl containing 1 or 2 heteroatoms selected from N, O or S.

62. The compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of claim 59, wherein, R₂ₐ is selected from any moiety in the Table 1 as shown in the description.

63. The compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of claim 59, wherein, R_{S1} at each occurrence is independently selected from deuterium, halogen, —C₁₋₆alkyl, haloC₁₋₆alkyl, —CN, —NH₂, —NH(C₁₋₃alkyl), —N(C₁₋₃alkyl)₂, —OH, —OC₁₋₃alkyl, —SH, —SC₁₋₃alkyl, —C(═O)C₁₋₃alkyl, —C(═O)OH, —C(═O)OC₁₋₃alkyl, —OC(═O)C₁₋₃alkyl, 3-6 membered cycloalkyl, 3-6 membered heterocyclyl or 5-6 membered heteroaryl; wherein said —C₁₋₆alkyl, haloC₁₋₆alkyl, 3-6 membered cycloalkyl, 3-6 membered heterocyclyl or 5-6 membered heteroaryl is optionally independently substituted with one or more substituents selected from deuterium, halogen, —C₁₋₆alkyl, haloC₁₋₆alkyl, —CN, —NH₂, —NH(C₁₋₃alkyl), —N(C₁₋₃alkyl)₂, —OH, —OC₁₋₃alkyl, —SH, —SC₁₋₃alkyl, —C(═O)C₁₋₃alkyl, —C(═O)OH, —C(═O)OC₁₋₃ alkyl, —OC(═O)C₁₋₃alkyl, 3-6 membered cycloalkyl or 3-6 membered heterocyclyl;
Optionally, two R_{S1} together with the carbon atom to which they are both attached form 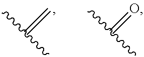
a 3-6 membered carbocyclic ring or a 3-6 heterocyclic ring; wherein, said 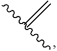
3-10 membered carbocylic ring or 3-10 heterocyclic ring is optionally substituted with one or more substitutents selected from deuterium, halogen, —C₁₋₆alkyl, haloC₁₋₆alkyl, —CN, —NH₂, —NH(C₁₋₃alkyl), —N(C₁₋₃alkyl)₂, —OH, —OC₁₋₃alkyl, —SH, —SC₁₋₃alkyl, —C(═O)C₁₋₃alkyl, —C(═O)OH, —C(═O)OC₁₋₃alkyl, —OC(═O)C₁₋₃alkyl, 3-6 membered cycloalkyl or 3-6 membered heterocyclyl;
Optionally, two adjacent R_{S1} together with the carbon atoms to which they are respectively attached form a 3-6 membered carbocyclic ring, or a 3-6 membered heterocyclic ring, wherein, each of rings is independently optionally substituted with one or more substitutents selected from deuterium, halogen, —C₁₋₆alkyl, haloC₁₋₆alkyl, —CN, —NH₂, —NH(C₁₋₃alkyl), —N(C₁₋₃alkyl)₂, —OH, —OC₁₋₃alkyl, —SH, —SC₁₋₃alkyl, —C(═O)C₁₋₃alkyl, —C(═O)OH, —C(═O)OC₁₋₃alkyl, —OC(═O)C₁₋₃alkyl, 3-6 membered cycloalkyl or 3-6 membered heterocyclyl.

64. The compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of claim 59, wherein, the moiety of 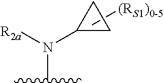
is selected from the moiety in the Table 2 as shown in the description.

65. The compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of claim 59, wherein, the moiety of 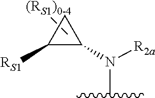
is selected from the moiety in the Table 3 as shown in the description.

66. The compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of claim 59, wherein, R_{S5} at each occurrence is independently selected from deuterium, halogen, —C₁₋₆alkyl, haloC₁₋₆alkyl, haloC₁₋₆alkoxy, —C₂₋₃alkenyl, —CN, —N(R₆₆)₂, —OR₆₆, —SR₆₆, —C(═O)R₆₇, —C(═O)OR₆₆, —OC(═O)R₆₇, —C(═O)N(R₆₆)₂, —NR₆₆C(═O)R₆₇, —OC(═O)OR₆₆, —NR₆₆C(═O)OR₆₆, —OC(═O)N(R₆₆)₂, —NR₆₆C(═O)N(R₆₆)₂, 3-8 membered cycloalkyl, 4-8 membered heterocyclyl containing 1, 2 or 3 heteroatoms selected from N, O or S or 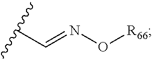
wherein, said —C₁₋₆alkyl is substituted with 1, 2 or 3 substituents selected from deuterium, halogen, —C₁₋₆alkyl, haloC₁₋₆alkyl, haloC₁₋₆alkoxy, —CN, oxo, —N(R₆₈)₂, —OR₆₈, —C(═O)R₆₈, —C(═O)OR₆₈, —OC(═O)R₆₈, —C(═O)N(R₆₈)₂, —NR₆₈C(═O)R₆₉, —OC(═O)OR₆₈, —NR₆₈C(═O)OR₆₉, —OC(═O)N(R₆₈)₂, —OC(═S)N(R₆₈)₂, —NR₆₈C(═O)N(R₆₈)₂, —NR₆₈S(═O)₂R₆₉, 3-6 membered cycloalkyl or 4-6 membered heterocyclyl; said 4-8 membered heterocyclyl is substituted with 1, 2 or 3 substituents selected from deuterium or —OR₆₈; said haloC₁₋₆alkyl is substituted with 1, 2 or 3 substituents selected from deuterium, —OR₆₈ or —C(═O)OR₆₈; said —C₂₋₃alkenyl is substituted with 1 substituents selected from deuterium or —C(═O)NR₆₈R₆₉;
Optionally, two R_{S5} together with the carbon atom to which they are both attached form 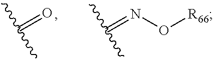
Each of (R₆₆ or R₆₇) is independently selected from hydrogen; deuterium; —C₁₋₆alkyl; haloC₁₋₆alkyl; or —C₁₋₆alkyl substituted with 1 or 2 substituents selected from —C(═O)N(C₁₋₆alkyl)₂, —OC₁₋₆alkyl, —C(═O)OC₁₋₆alkyl, —NHC₁₋₆alkyl or —N(C₁₋₆alkyl)₂;
Each of (R₆₈ or R₆₉) is independently selected from hydrogen; deuterium; —C₁₋₆alkyl; haloC₁₋₆alkyl; 5 membered heteroaryl; cyclopropyl; cyclopentyl; cyclohexyl; 5 membered heterocyclyl; 6 membered heterocyclyl; 5-membered heteroaryl; 6 membered heteroaryl; or —C₁₋₆alkyl substituted with 1, or 2 substituents selected from deuterium, —OC₁₋₆alkyl, —NHC₁₋₆alkyl, —N(C₁₋₆alkyl)₂ or —C(═O)N(C₁₋₆alkyl)₂; wherein said 5 membered heteroaryl, cyclopropyl, cyclopentyl, cyclohexyl, 5 membered heterocyclyl, 6 membered heterocyclyl, 5-membered heteroaryl or 6 membered heteroaryl is optionally substituted with 1 or 2 substitutents selected from deuterium, —C₁₋₃alkyl, —OH, —CN, —NH₂, —NH(C₁₋₃alkyl), —N(C₁₋₃alkyl)₂, —OC₁₋₃alkyl or cyclopropyl;
Optionally, two R₆₆ together with the nitrogen atom to which they are both attached form a 3-6 membered heterocyclic ring;
Optionally, two R₆₈ together with the nitrogen atom to which they are both attached form a 3-6 membered heterocyclic ring.

67. The compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of claim 59, wherein, R_{S5} at each occurrence is independently selected from deuterium, —F, —Cl, —CH₃, —CH₂CH₃, —CH₂CH₂CH₃, —CH(CH₃)₂, —CH═CH₂, —C═CH, —C═CCH₃, —C═CD, —CH₂C═CH, —CH₂F, —CHF₂, —CF₃, —CH₂CF₃, —CH₂CHF₂, —CH₂CH₂F, —CH₂CH₂CH₂F, —OH, —CH₂OH, —CH₂CH₂OH, —OCH₃, —OC(CH₃)₂, —OCH₂CH₃, —OCH(CH₃)₂, —OCF₃, —SH, —SCH₃, —SCF₃, —C(═O)CF₃, —CN, —NH₂, —N(CH₃)₂, —NHCH₂CH₃, —CH₂N(CH₃)₂, —NHC(═O)CH₃, —NHC(═O)OCH₃, —CH₂NHC(═O)OCH₃, —OC(═O)NHCH₃, —OC(═O)N(CH₃)₂, —CH₂OC(═O)N(CH₃)₂, —CH₂OC(═O)NHCH₃, —NHC(═O)N(CH₃)₂, —CH₂NHC(═O)N(CH₃)₂, —CH₂NHC(═O)CH₃, —CH₂OCH₃, or

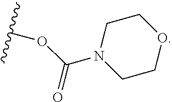 [SMILES: *OC(=O)N1CCOCC1]

68. The compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of claim 59, wherein, q₅ is selected from 0 or 1.

69. The compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of claim 59, wherein, the moiety of 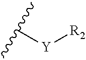
is selected from any moiety in the Table 4 as shown in the description.

70. The compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of claim 59, wherein, R₄ is selected from any moiety in the Table 6 as shown in the description;
Wherein, each moiety in the Table 6 is independently optionally substituted with 1, 2, 3, 4, 5 or 6 R₄₁.

71. The compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of claim 59, wherein, R₄₁ is independently selected from —F, —Cl, —C₁₋₃alkyl, haloC₁₋₃alkyl, haloC₁₋₃alkoxy, —C₂₋₃alkenyl, —C₂₋₃alkynyl, —CN, —NH₂, —NH(C₁₋₃alkyl), —N(C₁₋₃ alkyl)₂, —OH, —O(C₁₋₃alkyl), —SH, —S(C₁₋₃alkyl), —S(═O)H, —S(═O)(C₁₋₃alkyl), 3-6 membered cycloalkyl or 3-6 membered heterocyclyl, wherein said —C₁₋₃alkyl, haloC₁₋₃alkyl, haloC₁₋₃alkoxy, —C₂₋₃alkenyl, —C₂₋₆alkynyl, —NH₂, —SH, 3-6 membered cycloalkyl or 3-6 membered heterocyclyl is independently optionally substituted with 1, 2 or 3 R₄₂;
Each of R₄₂ is independently selected from —F; —C₁₋₃alkyl; haloC₁₋₃alkyl; —CN; —OH; —NH₂; —NH(C₁₋₃alkyl); —N(C₁₋₃alkyl)₂; —OC₁₋₃alkyl; 3-6 membered cycloalkyl; or —C₁₋₃alkyl substituted with 1, 2 or 3 substituents selected from —F, haloC₁₋₃alkyl, —CN, —OH, —NH₂, —NH(C₁₋₃alkyl), —N(C₁₋₃alkyl)₂ or —OC₁₋₃alkyl.

72. The compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of claim 59, wherein, R₄ is selected from any moiety in the Table 9 as shown in the description.

73. The compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of claim 59, wherein, R₅₁ is selected from hydrogen, deuterium, —Cl, —CN, —CH₃, —CHF₂, —CH₂F, —CF₃, —CH₂OH, —CH₂CH₃, —OCH₃, —OCH₂CH₃, —SCH₃, —NHCH₃, —N(CH₃)₂, —OCF₃, —CN, —CH₂CN, —COOH, —CONH₂, —COOCH₃,

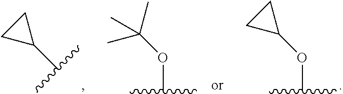

74. The compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of claim 59, wherein, R₅₂ is selected from —F.

75. The compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of claim 59, wherein, the prodrug is selected from any one of the following formulas: 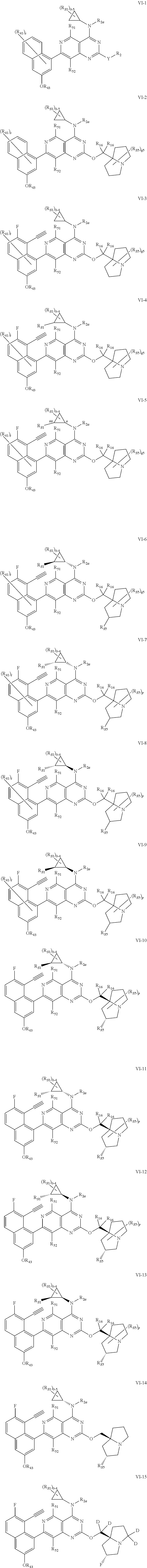
R₄₃ at each occurrence is independently selected from 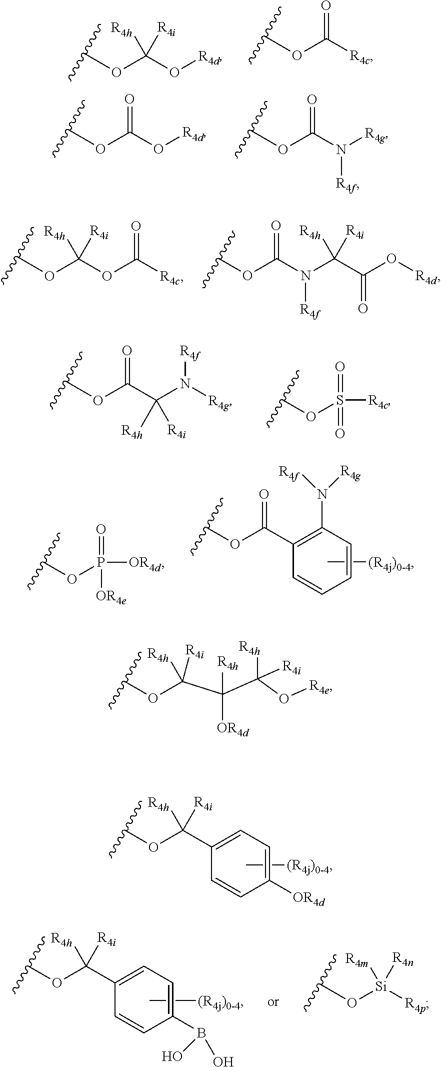
R_{4c} is selected from hydrogen, —C₁₋₃₀alkyl, —C₂₋₃₀alkenyl, —C₂₋₃₀alkynyl, —C₀₋₆alkylene-(3-20 membered carbocyclyl), —C₀₋₆alkylene-(3-20 membered heterocyclyl), —C₀₋₆alkylene-(6-10 membered aryl) or —C₀₋₆alkylene-(5-10 membered heteroaryl), each of which is independently substituted with one or more R₄ⱼ;
R_{4d} and R₄ₑ are each selected from hydrogen, —C₁₋₃₀alkyl, —C₂₋₃₀alkenyl, —C₂₋₃₀alkynyl, —C(═O)C₁₋₆alkyl, —C₀₋₆alkylene-(3-20 membered carbocyclyl), —C₀₋₆alkylene-(3-20 membered heterocyclyl), —C₀₋₆alkylene-(6-10 membered aryl) or —C₀₋₆alkylene-(5-10 membered heteroaryl), each of which is independently substituted with one or more R₄ⱼ;
R_{4f} and R_{4g} are each selected from hydrogen, —C₁₋₃₀alkyl, —C₂₋₃₀alkenyl, —C₂₋₃₀alkynyl, —C(═O)C₁₋₆alkyl, —C₀₋₆alkylene-(3-20 membered carbocyclyl), —C₀₋₆alkylene-(3-20 membered heterocyclyl), —C₀₋₆alkylene-(6-10 membered aryl) or —C₀₋₆alkylene-(5-10 membered heteroaryl), each of which is independently substituted with one or more R₄ⱼ;
R₄ₕ, R₄ᵢ, R₄ₘ, R₄ₙ and R₄ₚ are each selected from hydrogen, halogen, —C₁₋₆alkyl, haloC₁₋₆alkyl, haloC₁₋₆ alkoxy, —C₂₋₆alkenyl, —C₂₋₆alkynyl, —CN, —NH₂, —NH(C₁₋₆alkyl), —N(C₁₋₆alkyl)₂, oxo, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl or 5-10 membered heteroaryl; wherein, said —C₁₋₆alkyl, —C₂₋₆alkenyl, —C₂₋₆alkynyl, 3-10 membered cycloalkyl, 3-10 membered heterocyclyl, 6-10 membered aryl or 5-10 membered heteroaryl is optionally substituted with one or more substituents selected from halogen, —C₁₋₆alkyl, haloC₁₋₆alkyl, haloC₁₋₆ alkoxy, —C₂₋₆alkenyl, —C₂₋₆alkynyl, —CN, —NH₂, —NH(C₁₋₆alkyl), —N(C₁₋₆alkyl)₂, oxo, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl or 5-10 membered heteroaryl;
Optionally, R_{4f} and R_{4g} together with the atoms to which they are respectively attached form a 4-10 membered heterocyclyl ring, said 4-10 membered heterocyclyl ring optionally further contains 1 or 2 heteroatoms selected from N, O, S, S(═O) or S(═O)₂ and optionally substituted with one or more R₄;
Optionally, R_{4f} and R₄ₕ together with the atoms to which they are respectively attached form a 4-10 membered heterocyclyl ring, said 4-10 membered heterocyclyl ring optionally further contains 1 or 2 heteroatoms selected from N, O, S, S(═O) or S(═O)₂ and optionally substituted with one or more R₄;
R₄ⱼ at each occurrence is independently selected from halogen, —C₁₋₆alkyl, haloC₁₋₆alkyl, haloC₁₋₆alkoxy, —C₂₋₆alkenyl, —C₂₋₆alkynyl, —CN, oxo, —NO₂, —NH₂, —NH(C₁₋₆alkyl), —N(C₁₋₆alkyl)₂, —OH, —O(C₁₋₆alkyl), —SH, —S(C₁₋₆ alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆ alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl, or 5-10 membered heteroaryl, wherein said —C₁₋₆alkyl, haloC₁₋₆alkyl, haloC₁₋₆alkoxy, —C₂₋₆alkenyl, —C₂₋₆alkynyl, 3-10 membered cycloalkyl, 3-10 membered heterocyclyl, 6-10 membered aryl or 5-10 membered heteroaryl is independently optionally substituted with 1, 2 or 3 substituents selected from halogen; —C₁₋₆alkyl; haloC₁₋₆alkyl; —CN; oxo; —OH; —NH₂; —NH(C₁₋₆alkyl); —N(C₁₋₆alkyl)₂; —OC₁₋₆alkyl; or —C₁₋₆alkyl substituted with 1, 2 or 3 substituents selected from halogen, haloC₁₋₆alkyl, —CN, —OH, —NH₂, —NH(C₁₋₆alkyl), —N(C₁₋₆alkyl)₂ or —OC₁₋₆alkyl;
Each of (heterocyclyl and heteroaryl) at each occurrence is independently contain 1, 2, 3 or 4 heteroatoms selected from N, O, S, S(═O) or S(═O)₂.

76. The compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of claim 59, wherein, the moiety of 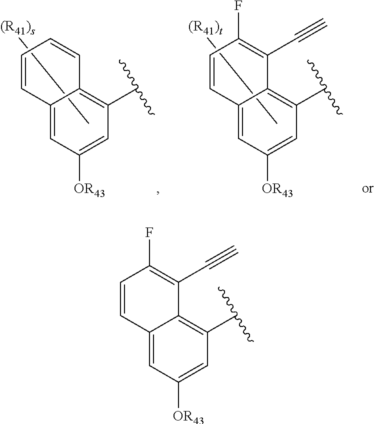
is selected from any moiety in the Table 12 as shown in the description.

77. The compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of claim 59 selected from any compound in the Table 13 as shown in the description.

78. A pharmaceutical composition, comprising a therapeutically effective amount of the compound of Formula (IB), the stereoisomer thereof, the pharmaceutically acceptable salt thereof, the pharmaceutically acceptable salt of the stereoisomer thereof, the prodrug thereof, the deuterated molecule thereof or the conjugated form thereof of claim 59, and a pharmaceutically acceptable excipient.
